(19) United States
(12) Patent Application Publication
(10) Pub. No.: US 2023/0031954 A1
(43) Pub. Date: Feb. 2, 2023

(54) TETRACYCLINE COMPOUNDS AND METHODS OF TREATMENT

(71) Applicant: Tetraphase Phamaceuticals, Inc., Watertown, MA (US)

(72) Inventors: Xiao-Yi Xiao, Lexington, MA (US); Jacques P. Dumas, Carlisle, MA (US); Diana K. Hunt, Cambrigde, MA (US); Cuixiang Sun, Arlington, MA (US); Peng Zhao, Franklin Park, NJ (US)

(21) Appl. No.: 16/328,559

(22) PCT Filed: Aug. 30, 2017
(86) PCT No.: PCT/US2017/049462

§ 371 Date: Feb. 26, 2019

Related U.S. Application Data
(60) Provisional application No. 62/381,383, filed on Aug. 30, 2016.
(60) Provisional application No. 62/437,533, filed on Dec. 21, 2016.

(51) Int. Cl. A61K 31/65

(52) U.S. Cl. CPC A61K 31/65

(57) ABSTRACT

The present invention is directed to methods of treating hematological cancers, such as acute myleiod leukemia, with tetracyclines, or a pharmaceutically acceptable salt thereof.

### RELATED APPLICATIONS

This application is the U.S. National Stage of International Application No. PCT/US2017/049462, filed Aug. 30, 2017, which designates the U.S., published in English, and claims the benefit of U.S. Provisional Application No. 62/381,383, filed on Aug. 30, 2016 and, 62/437,533, filed Dec. 21, 2016. The entire teachings of the above applications are incorporated herein by reference.

### BACKGROUND OF THE INVENTION

Hematological malignancies are cancers that affect the blood and lymph system. Some types of hematologic malignancies include: Multiple myeloma, Hodgkin lymphoma, Non-Hodgkin lymphoma and Leukemia. The cancer may begin in blood-forming tissue (e.g., bone marrow), or in the cells of the immune system. For example, leukemia originates in blood-forming tissue. Leukemia is characterized by the uncontrolled growth of blood cells, usually white blood cells (leukocytes), in the bone marrow. White blood cells are a fundamental component of the body's immune response. The leukemia cells crowd out and replace normal blood and marrow cells.

There are four main types of leukemia: Acute myeloid leukemia (AML); Chronic myeloid leukemia (CML); Acute lymphocytic leukemia (ALL); and Chronic lymphocytic leukemia (CLL). The primary differences between the four main types of leukemia have to do with their rates of progression and where the cancer develops. Acute myeloid leukemia (AML), also known as acute myelogenous leukemia, acute myeloblastic leukemia, acute granulocytic leukemia or acute nonlymphocytic leukemia, is a fast-growing form of cancer of the blood and bone marrow. AML is the most common type of acute leukemia. It occurs when the bone marrow begins to make blasts, cells that have not yet completely matured. These blasts normally develop into white blood cells. However, in AML, these cells do not develop and are unable to ward off infections. In AML, the bone marrow may also make abnormal red blood cells and platelets. The number of these abnormal cells increases rapidly, and the abnormal (leukemia) cells begin to crowd out the normal white blood cells, red blood cells and platelets that the body needs.

The standard treatment for AML includes remission-induction treatment consisting of administration of the chemotherapeutic agents cytarabine and daunorubicin (7+3). This treatment has been the standard of care for decades. Few other therapeutic approaches for malignant disease have remained so unchanged for such a long period. In addition, the co-morbidities and high susceptibility to treatment-related toxicity still limit treatment success. Despite advances in treatment strategies for hematological cancer there remains a need to identify novel, potent and well-tolerated tetracyclines, particularly for the treatment of leukemias, such as AML, to be used either as a single agent or in combination with other anti-neoplastic agents. are needed in order to maximize the therapeutic benefit and minimize treatment-related toxicity.

### SUMMARY OF THE INVENTION

A first embodiment of the present invention is directed to a method of treating a hematological cancer in a subject in need thereof comprising administering to the subject an effective amount of a compound represented by:

### Structural Formula (I) or (I′):

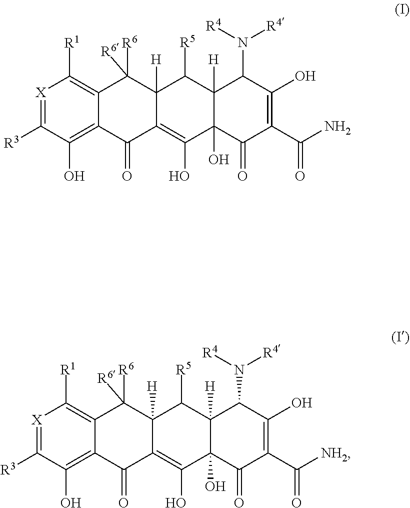

### Structural Formula (II) or (II′):

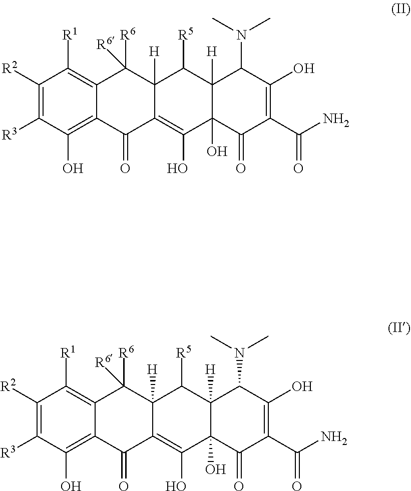

or

### Structural Formula (III) or (III′):

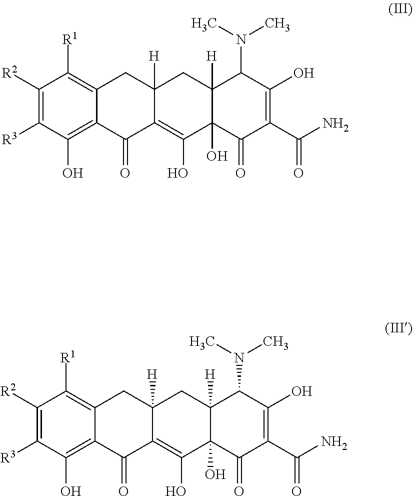

or a pharmaceutically acceptable salt thereof, wherein the variables are as defined and described herein.

Another embodiment of the present invention is the use of a compound represented by Structural Formula (I), (I′), (II), (II′), (III) or (III′) or a pharmaceutically acceptable salt thereof, for the manufacture of a medicament for treating a hematological cancer. In one aspect the hematogical malignancy is leukemia. In a specific aspect, the leukemia is AML.

Another embodiment of the present invention is a compound represented by Structural Formula (I), (I′), (II), (II′), (III) or (III′), or a pharmaceutically acceptable salt thereof, four use in treating hematological cancers. In one aspect the hematogical malignancy is leukemia. In a specific aspect, the leukemia is AML.

Another embodiment of the present invention is a method of treating a hematological cancer comprising administering to a subject in need of treatment an effective amount of a compound represented by any one of structural formulas (X) or (X-1)

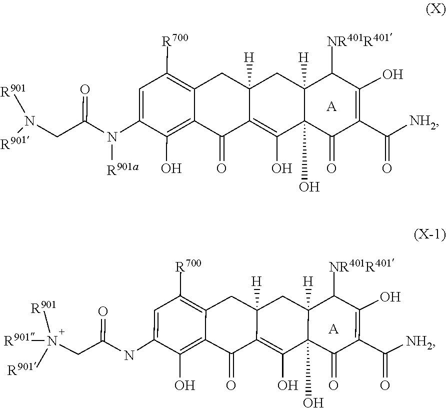

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by any one of structural formulas (XI), or a pharmaceutically acceptable salt thereof, or a pharmaceutically acceptable composition thereof,

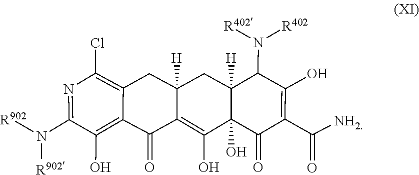

Another embodiment of the present invention is a compound represented by structural formula (XII), or a pharmaceutically acceptable salt thereof:

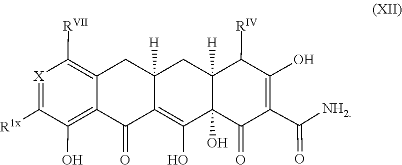

Another embodiment of the present invention is a method of treating a hematological cancer comprising administering to a subject in need of treatment an effective amount of a compound represented by structural formula (XII), or a pharmaceutically acceptable salt thereof, or a pharmaceutically acceptable composition thereof.

Another embodiment of the present invention is a hematological cancer comprising administering to a subject in need of treatment an effective amount of a compound represented by the following structural formula

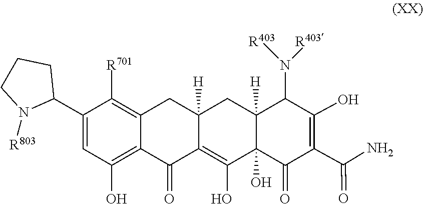

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by the following structural formula:

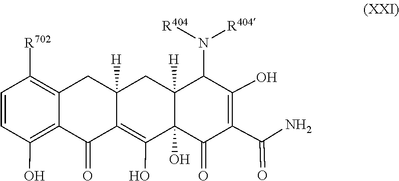

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by any one of structural formulas

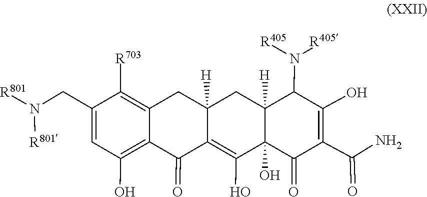

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by the following structural formula

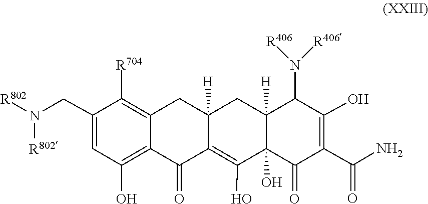

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by the following structural formula

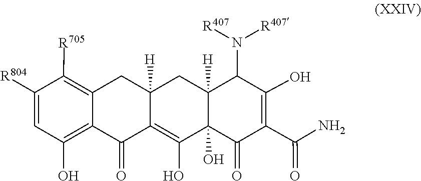

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by the following structural formula

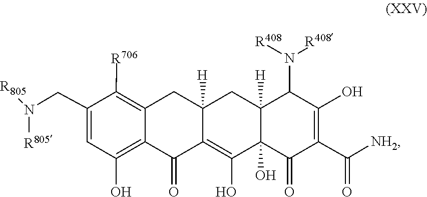

or a pharmaceutically acceptable salt thereof, or a pharmaceutically acceptable composition thereof.

Another embodiment of the present invention is any compound represented by structural formula (XIII):

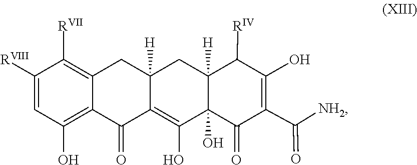

or a pharmaceutically acceptable salt thereof, or a pharmaceutically acceptable composition thereof.

Another embodiment of the present invention is a method of treating a hematological cancer comprising administering to a subject in need of treatment an effective amount of a compound represented by structural formula (XIII), or a pharmaceutically acceptable salt thereof, or a pharmaceutically acceptable composition thereof.

Another embodiment of the present invention is a compound represented by any one of structural formulas (XIV) or (XV):

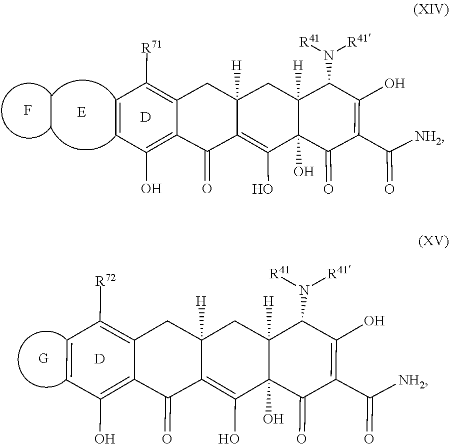

or a pharmaceutically acceptable salt thereof.

Another embodiment of the present invention is a pharmaceutical composition comprising a pharmaceutically acceptable carrier or diluent and a compound of any of the foregoing embodiments.

Another embodiment of the present invention is a method of treating a subject suffering from a hematological tumor, comprising administering to the subject a therapeutically effective amount of any compound of a pharmaceutical composition of the foregoing embodiments.

Another embodiment of the present invention is a method for treating a bacterial infection in a subject in need thereof, comprising administering to the subject a therapeutically effective amount of a compound represented by any one of structural formulas XIV or XV or a compound of Formulas XIII or XII.

### BRIEF DESCRIPTION OF THE DRAWINGS

FIG. 1 depicts a Western Blot that shows levels of COX1, COX4 and actin in MV4-11 cells treated with Compound 1 as described in Example 2.

FIG. 2 depicts a Western Blot that shows levels of COX1, COX4 and actin in MV4-11 cells treated with Compound 2 as described in Example 2.

FIG. 3 depicts a Western Blot that shows levels of COX1, COX4 and actin in MV4-11 cells treated with Compound 3a as described in Example 2.

FIG. 4 depicts a Western Blot that shows levels of COX1, COX4 and actin in MV4-11 cells treated with Compound 4a as described in Example 2.

FIG. 5 depicts a Western Blot that shows levels of COX1, COX4 and actin in MV4-11 cells treated with Compound 5 as described in Example 2.

FIG. 6 is a graph showing the dose-response fitting functions for cytarabine (top panel) and Compound 3a (bottom panel). The X-axis is the concentration of compound tested and the Y-axis is the Normalized effect-Survival % (count/E0). Normalization was done after modeling regarding the estimated basal (E0) parameter.

FIG. 7A is a graph of Tumor Volume vs. Days After Start of Treatment (Compound 3a at dose 1 and dose 2 of Table 1C) of CB17 SCID mice testing in the xenograft model using MV4-11 leukemia model.

FIG. 7B is a graph of Body Weight Change (%) vs. Days After Start of Treatment (Compound 3a at does 1 and dose 2 of Table 1C) of CB17 SCID mice testing in the xenograft model using MV4-11 leukemia model.

FIG. 7C is a graph of Tumor Volume vs. Days After Start of Treatment (Compound 4a at dose 1 and dose 2 of Table 1C) of CB17 SCID mice testing in the xenograft model using MV4-11 leukemia model.

FIG. 7D is a graph of Body Weight Change (%) vs. Days After Start of Treatment (Compound 4a at does 1 and dose 2 of Table 1C) of CB17 SCID mice testing in the xenograft model using MV4-11 leukemia model.

FIG. 7E is a graph of Tumor Volume vs. Days After Start of Treatment (Compound 5 at dose 1 and dose 2 of Table 1C) of CB17 SCID mice testing in the xenograft model using MV4-11 leukemia model.

FIG. 7F is a graph of Body Weight Change (%) vs. Days After Start of Treatment (Compound 5 at does 1 and dose 2 of Table 1C) of CB17 SCID mice testing in the xenograft model using MV4-11 leukemia model.

FIG. 8 shows the dose-response results for Compound 3a in the Rat Heart Mitochondrial Translation Assay.

FIG. 9 shows the results for MV411 MT-COX1 (Cytochrome oxidase subunit 1, expressed in mitochrondria) expression. The X-axis (drug concentration) shows results from left to right on the page as follows: Compound 3a, Tigecycline and Cytarabine.

FIG. 10 shows the results for MV411 COX-IV expression (Cytochrome oxidase subunit 4, expressed in nucleus). The X-axis (drug concentration) shows results from left to right on the page as follows: Compound 3a, Tigecycline and Cytarabine.

FIG. 11 shows the results for MV411 PIG3 expression (TP₅₃I₃-a p53 responsive protein, expression induced in response to p53 activation, role associated with response to oxidative stress). The X-axis (drug concentration) shows results from left to right on the page as follows: Compound 3a, Tigecycline and Cytarabine.

FIG. 12 shows the results for MV411 BAX expression (pro-apoptotic protein expression induce by p53 activation, forms a heterodimer with BCL2 to induce apoptosis). The X-axis (drug concentration) shows results from left to right on the page as follows: Compound 3a, Tigecycline and Cytarabine.

FIG. 13 shows the results of CDKN2A expression (also known as p14^{ARF} or ARF-nuclear gene, translation regulated by cMyc, functions to stabilize/activate p53 by binding and sequestering Mdm2). The X-axis (drug concentration) shows results from left to right on the page as follows: Compound 3a, Tigecycline and Cytarabine.

FIG. 14A through FIG. 14E, collectively, represent a table of Minimal Inhibitory Concentrations (MIC) values, in μg/mL, of the example compounds disclosed in the preset application.

FIG. 15A through FIG. 15M, collectively, represent a table of “Inhibitory Concentrations 50%” (IC₅₀) values of example compounds disclosed in the present application measured against the indicated hematological cancer cell lines.

FIG. 16A through FIG. 16F, collectively, represent a table of “Inhibitory Concentrations 50%” (IC₅₀) values of example compounds disclosed in the present application measured against the indicated hematological cancer cell lines.

FIG. 17A through FIG. 17D, collectively, represent a table of “Inhibitory Concentrations 50%” (IC₅₀) values of example compounds disclosed in the present application measured against the indicated hematological cancer cell lines.

### DETAILED DESCRIPTION OF THE INVENTION

The present invention relates to a method of treating a hematological cancer in a subject in need thereof. The method comprises administering to the subject an effective amount of a compound represented by any one of Structural Formulas (I), (I′), (II), (II′), (III) or (III′) or a pharmaceutically acceptable salt thereof. The variables in Structural Formulas (I), (I′), (II), (II′), (III) or (III′) are described herein in the following paragraphs. It is understood that the invention encompasses all combinations of the substituent variables (i.e., R¹, R², R³, etc.) defined herein.

In a first embodiment of the invention, the compound administered in the method of treating a hematological cancer is a compound having Structural Formula (I) or (T′):

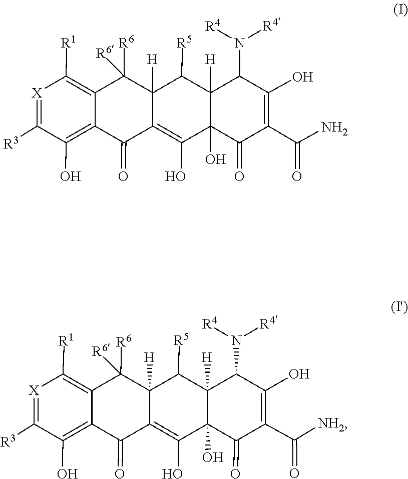

or a pharmaceutically acceptable salt thereof, wherein:

X is selected from N and C(R²);
each of R¹, R², R³, R⁵ and R⁶ is independently selected from hydrogen, halo, —(C₁-C₆ alkyl), —OR^{A}, —C(O)NR^{B}R^{B′}, —NR^{B}R^{B′}, —S(O)₀₋₂R^{C}, —(C₀-C₆ alkylene)-carbocyclyl, and —(C₀-C₆ alkylene)-heterocyclyl; or
R¹ and R² are optionally taken together with atoms to which they are bound to form a carbocyclyl or heterocyclyl ring; or
R² and R³ are optionally taken together with atoms to which they are bound to form a carbocyclyl or heterocyclyl ring;
R⁴ is selected from hydrogen, —(C₁-C₆ alkyl), —(C₀-C₆ alkylene)-carbocyclyl, and —(C₀-C₆ alkylene)-heterocyclyl;
R^{4′} is selected from hydrogen, —(C₁-C₆ alkyl), S(O)₁₋₂R^{C}, —(C₀-C₆ alkylene)-carbocyclyl, —(C₀-C₆ alkylene)-heterocyclyl, —C(O)—(C₁-C₆ alkyl), and —C(O)—(C₁-C₆ alkyl)-NR^{D}R^{E}; or
R⁴ and R^{4′} are optionally taken together with the nitrogen atom to which they are commonly bound to form a 4-8 membered ring optionally comprising 1-2 additional heteroatoms independently selected from N, O and S;
R^{6′} is selected from hydrogen, —(C₁-C₆ alkyl) and —(C₃-C₆ cycloalkyl);
each R^{A} is independently selected from hydrogen, —(C₁-C₆ alkyl), —(C₀-C₆ alkylene)-carbocyclyl, —(C₀-C₆ alkylene)-heterocyclyl, —C(O)—(C₁-C₆ alkyl), —C(O)—(C₀-C₆ alkylene)-carbocyclyl, —C(O)—(C₀-C₆ alkylene)-heterocyclyl, and —C(O)N(RD)(R^{E});
each R^{B} and each R^{B′} is independently selected from hydrogen, —(C₁-C₆ alkyl), —(C₁-C₆ haloalkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, and —C(O)—(C₀-C₆ alkylene)-N(R^{D})(R^{E});
each R^{C} is independently selected from —(C₁-C₆ alkyl), —(C₀-C₆ alkylene)-carbocyclyl and —(C₀-C₆ alkylene)-heterocyclyl; and
each R^{D} and each R^{E} is independently selected from hydrogen, —(C₁-C₆ alkyl), —(C₀-C₆ alkylene)-carbocyclyl, and —(C₀-C₆ alkylene)-heterocyclyl,

wherein any alkyl, alkylene, carbocyclyl or heterocyclyl portion of R¹, R², R³, R⁴, R^{4′}, R⁵, R⁶, R^{6′}, R^{A}, R^{B}, R^{B′}, R^{C}, R^{D}, or R^{E} or formed by taking R¹ and R², R² and R³, or R⁴ and R^{4′} together is optionally and independently substituted.

In a first aspect of the first embodiment:
any alkyl, or alkylene portion of R¹, R², R³, R⁴, R^{4′}, R⁵, R⁶ is optionally and independently substituted with one or more substituents independently selected from halo, ═O, OR^{A}, NR^{B}R^{B′}, and S(O)₀₋₂R^{C};
any alkyl or alkylene portion of R^{6′}, R^{A}, or R^{C} is optionally and independently substituted with one or more fluoro;
any carbocyclyl or heterocyclyl portion of any of R¹, R², R³, R⁴, R^{4′}, R⁵, R⁶, or any ring formed by taking together R¹ and R², R² and R³ or R⁴ and R^{4′} is optionally and independently substituted on a carbon atom with one or more substituents independently selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C};
any heterocyclyl portion of any of R¹, R², R³, R⁴, R^{4′}, R⁵, R⁶, or any ring formed by taking together R¹ and R², R² and R³ or R⁴ and R^{4′} is optionally and independently substituted on a substitutable nitrogen atom with R^{F};
each R^{F} is independently selected from —(C₁-C₆ alkyl), —(C₁-C₆ haloalkyl), —(C₁-C₆ hydroxyalkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, —(C₀-C₆ alkylene)-C(O)₂—(C₁-C₆ alkyl), —(C₁-C₆ alkylene)-NR^{B}R^{B′} and —C(O)N(R^{D})(R^{E});
any carbocyclyl or heterocyclyl portion of R^{A}, R^{B}, R^{B′}, R^{C}, R^{D}, R^{E}, R^{F}, any cycloalkyl portion of R^{6′}, or any substituent of R¹, R², R³, R⁴, R^{4′}, R⁵, R⁶ is optionally and independently substituted on a carbon atom with a one or more substituents independently selected from fluoro, chloro, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂;
any heterocyclyl portion of R^{A}, R^{B}, R^{B′}, R^{C}, R^{D}, R^{E}, R^{F}, or any heterocyclyl substituent of R¹, R², R³, R⁴, R^{4′}, R⁵, or R⁶ is optionally substituted on a substitutable nitrogen atom with —C₁-C₄ alkyl, or —S(O)₁₋₂—(C₁-C₄ alkyl). The remaining variables are as described and defined in the first embodiment.

In a second aspect of the first embodiment, the compound is other than:

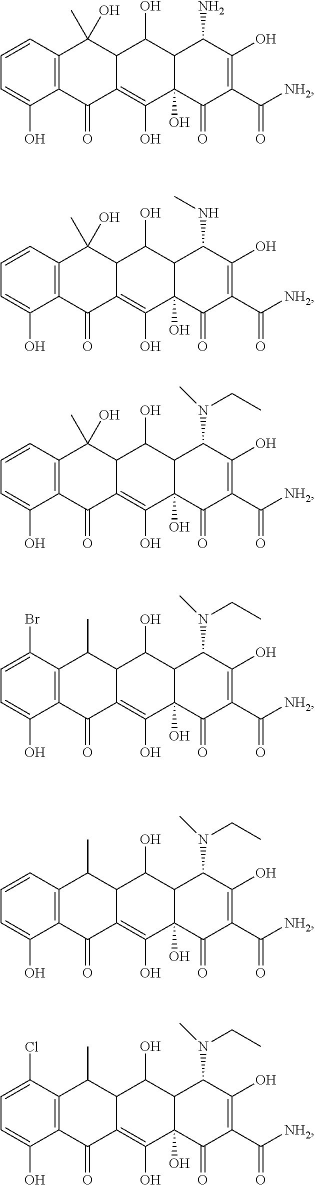

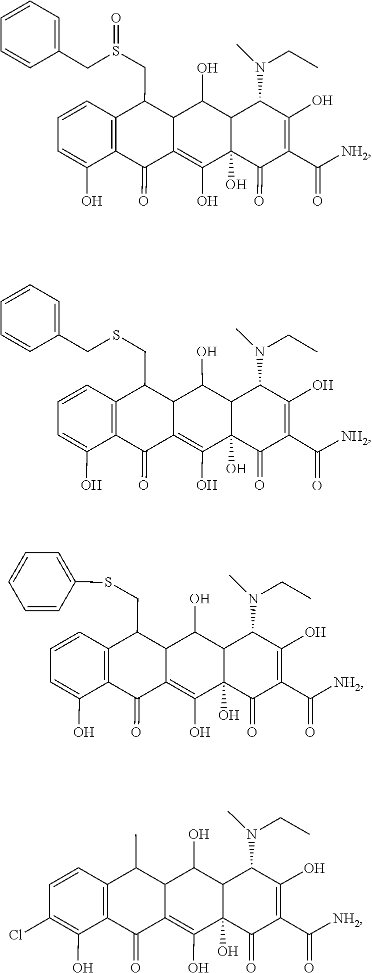 [SMILES: CCN(C)[C@@H]1C(O)=C(C(N)=O)C(=O)[C@@]2(O)C(O)=C3C(=O)c4c(O)cccc4C(CS(=O)Cc4ccccc4)C3C(O)C12 ; CCN(C)[C@@H]1C(O)=C(C(N)=O)C(=O)[C@@]2(O)C(O)=C3C(=O)c4c(O)cccc4C(CSCc4ccccc4)C3C(O)C12 ; CCN(C)[C@@H]1C(O)=C(C(N)=O)C(=O)[C@@]2(O)C(O)=C3C(=O)c4c(O)cccc4C(CSc4ccccc4)C3C(O)C12 ; CCN(C)[C@@H]1C(O)=C(C(N)=O)C(=O)[C@@]2(O)C(O)=C3C(=O)c4c(ccc(Cl)c4O)C(C)C3C(O)C12]

or a salt of any of the foregoing. The remaining variables are as described and defined in the first embodiment, or first aspect thereof.

In a third aspect of the first embodiment, each of R⁵, R⁶ and R^{6′} is hydrogen. The remaining variables are as described and defined in the first embodiment, or the first or second aspect thereof.

In a fourth aspect of the first embodiment, X is C(R²). The remaining variables are as described and defined in the first embodiment, or the first, second or third aspect thereof.

In a fifth aspect of the first embodiment:

X is selected from N and C(R²);

each of R¹, R², R³, R⁵ and R⁶ is independently selected from hydrogen, halo, —(C₁-C₆ alkyl), —OR^{A}, NR^{B}R^{B′}, —C(O)NR^{B}R^{B′}, S(O)₀₋₂R^{C}, —(C₀-C₆ alkylene)-carbocyclyl, and —(C₀-C₆ alkylene)-heterocyclyl; or

R¹ and R² are optionally taken together with atoms to which they are bound to form a carbocyclyl or heterocyclyl ring; or

R² and R³ are optionally taken together with atoms to which they are bound to form a carbocyclyl or heterocyclyl ring;

R⁴ is selected from hydrogen, —(C₁-C₆ alkyl), —(C₀-C₆ alkylene)-carbocyclyl, and —(C₀-C₆ alkylene)-heterocyclyl;

R^{4′} is selected from hydrogen, —(C₂-C₆ alkyl), S(O)₁₋₂R^{C}, —(C₀-C₆ alkylene)-carbocyclyl, —(C₀-C₆ alkylene)-heterocyclyl, —C(O)—(C₁-C₆ alkyl), and —C(O)—(C₁-C₆ alkyl)-NR^{D}R^{E}; or

R⁴ and R^{4′} are optionally taken together with the nitrogen atom to which they are commonly bound to form a 4-8 membered ring optionally comprising 1-2 additional heteroatoms independently selected from N, O and S;

R^{6′} is selected from hydrogen, —(C₁-C₆ alkyl) and —(C₃-C₆ cycloalkyl);

each R^{A} is independently selected from hydrogen, —(C₁-C₆ alkyl), —(C₀-C₆ alkylene)-carbocyclyl, —(C₀-C₆ alkylene)-heterocyclyl, —C(O)—(C₁-C₆ alkyl), —C(O)—(C₀-C₆ alkylene)-carbocyclyl, —C(O)—(C₀-C₆ alkylene)-heterocyclyl, and —C(O)N(R^{D})(R^{E});

each R^{B} and each R^{B′} is independently selected from hydrogen, —(C₁-C₆ alkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, and —C(O)N(R^{D})(R^{E});

each R^{C} is independently selected from —(C₁-C₆ alkyl), —(C₀-C₆ alkylene)-carbocyclyl and —(C₀-C₆ alkylene)-heterocyclyl; and

each R^{D} and each R^{E} is independently selected from hydrogen, —(C₁-C₆ alkyl), —(C₀-C₆ alkylene)-carbocyclyl, and —(C₀-C₆ alkylene)-heterocyclyl;
wherein any alkyl, alkylene, carbocyclyl or heterocyclyl portion of R¹, R², R³, R⁴, R^{4′}, R⁵, R⁶, R^{6′}, R^{A}, R^{B}, R^{B′}, R^{C}, R^{D}, or R^{E} or formed by taking R¹ and R², R² and R³, or R⁴ and R^{4′} together is optionally and independently substituted. The remaining variables are as described and defined in the first embodiment, or the first, second, third or fourth aspect thereof.

In a sixth aspect of the first embodiment:

any alkyl or alkylene portion of R¹, R², R³, R⁴, R^{4′}, R⁵, or R⁶ is optionally and independently substituted with one or more substituents independently selected from halo, ═O, OR^{A}, NR^{B}R^{B′}, and S(O)₀₋₂R^{C};

any alkyl or alkylene portion of R^{6′}, R^{A}, or R^{C}, is optionally and independently substituted with one or more fluoro;

any carbocyclyl or heterocyclyl portion of any of R¹, R², R³, R⁴, R^{4′}, R⁵, or R⁶, or any ring formed by taking together R¹ and R², R² and R³, or R⁴ and R^{4′} is optionally and independently substituted on a carbon atom with one or more substituents independently selected from halo, ═O, C₁-C₄ fluoroalkyl, C₁-C₄ alkyl, C₃-C₁₀ carbocyclyl, a 4-13 membered heterocyclyl, OR^{A}, NR^{B}R^{B′}, and S(O)₀₋₂R^{C};

any heterocyclyl portion of any of R¹, R², R³, R⁴, R^{4′}, R⁵, or R⁶, or any ring formed by taking together R¹ and R², R² and R³, or R⁴ and R^{4′} is optionally and independently substituted on a substitutable nitrogen atom with R^{F};

each R^{F} is independently selected from —(C₁-C₆ alkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, and —C(O)N(R^{D})(R^{E});

any carbocyclyl or heterocyclyl portion of R^{A}, R^{B}, R^{B′}, R^{C}, R^{D}, R^{E}, R^{F}, any cycloalkyl portion of R⁶, or any substituent of R¹, R², R³, R⁴, R^{4′}, R⁵, or R^{6′} is optionally and independently substituted on a carbon atom with a one or more substituents independently selected from halo, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂; and any heterocyclyl portion of R^{A}, R^{B}, R^{B′}, R^{C}, R^{D}, R^{E}, R^{F}, or any heterocyclyl substituent of R¹, R², R³, R⁴, R^{4′}, R⁵, or R⁶ is optionally substituted on a substitutable nitrogen atom with —C₁-C₄ alkyl, or —S(O)₁₋₂—(C₁-C₄ alkyl). The remaining variables are as described and defined in the first embodiment, or the first, second, third, fourth or fifth aspect thereof.

In a seventh aspect of the first embodiment, X is N. The remaining variables are as described and defined in the first embodiment, or the first, second, third, fourth, fifth or sixth aspect thereof.

In an eighth aspect of the first embodiment, R¹ is selected from hydrogen, halo, —(C₁-C₆ alkyl) optionally substituted with one or more halo, —NR^{B}R^{B′}, —C(O)NR^{B}R^{B′}, —OR^{A}, —(C₀-C₆ alkylene)-carbocyclyl, and —(C₀-C₆ alkylene)-heterocyclyl, wherein R^{A} is C₁-C₆ alkyl optionally substituted with one or more fluoro. The remaining variables are as described and defined in the first embodiment, or the first, second, third, fourth, fifth, sixth or seventh aspect thereof.

In a ninth aspect of the first embodiment, R³ is selected from hydrogen and —N(R^{B})(R^{B′}), wherein R^{B} is hydrogen. The remaining variables are as described and defined in the first embodiment, or the first, second, third, fourth, fifth, sixth, seventh or eighth aspect thereof.

In a tenth aspect of the first embodiment, the compound for use in treating a hematological cancer is selected from any of the compounds in the following tables or a pharmaceutically acceptable salt thereof:

| Compound No. | Compound Structure |
|---|---|
| S3-7-1-A (diastereomer A) S3-7-1-B (diastereomer B) | 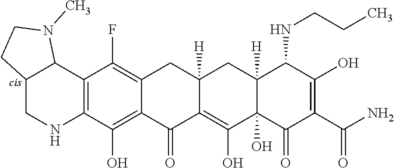 [SMILES: CCCN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-2 | 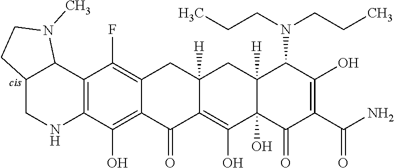 [SMILES: CCCN(CCC)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-3-A (diastereomer A) S3-7-3-B (diastereomer B) | 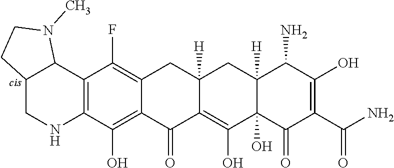 [SMILES: CN1CCC2CNc3c(O)c4c(c(F)c3C21)C[C@H]1C[C@H]2[C@H](N)C(O)=C(C(N)=O)C(=O)[C@@]2(O)C(O)=C1C4=O] |
| S3-7-4-A (diastereomer A) S3-7-4-B (diastereomer B) | 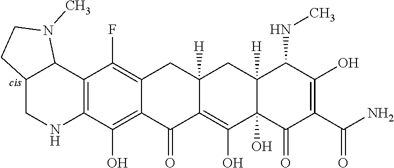 [SMILES: CN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-5 | 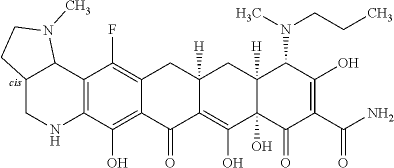 [SMILES: CCCN(C)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-6-A (diastereomer A) S3-7-6-B (diastereomer B) | 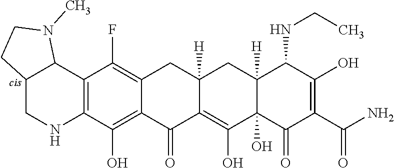 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-7-A (diastereomer A) S3-7-7-B (diastereomer B) | 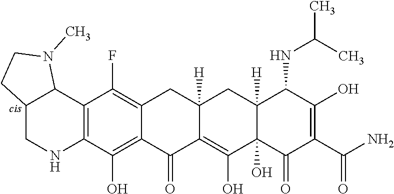 [SMILES: CC(C)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-8-A (diastereomer A) S3-7-8-B (diastereomer B) | 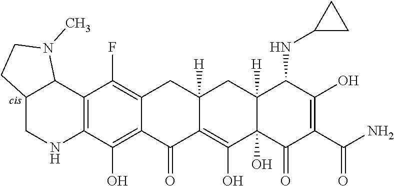 [SMILES: CN1CCC2CNc3c(O)c4c(c(F)c3C21)C[C@H]1C[C@H]2[C@H](NC3CC3)C(O)=C(C(N)=O)C(=O)[C@@]2(O)C(O)=C1C4=O] |
| S3-7-9-A (diastereomer A) S3-7-9-B (diastereomer B) | 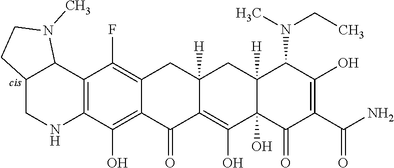 [SMILES: CCN(C)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-10-A (diastereomer A) S3-7-10-B (diastereomer B) | 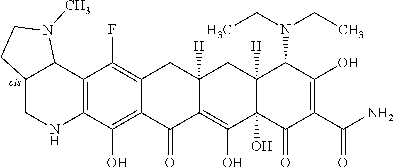 [SMILES: CCN(CC)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-11 | 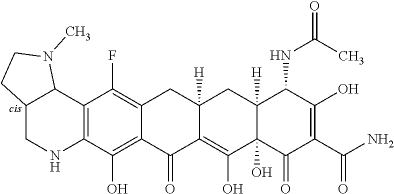 [SMILES: CC(=O)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-12 | 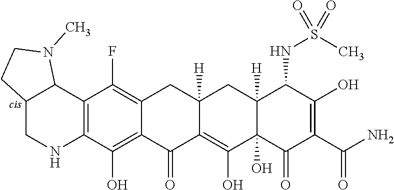 [SMILES: CN1CCC2CNc3c(O)c4c(c(F)c3C21)C[C@H]1C[C@H]2[C@H](NS(C)(=O)=O)C(O)=C(C(N)=O)C(=O)[C@@]2(O)C(O)=C1C4=O] |
| S3-7-13-A (diastereomer A) S3-7-13-B (diastereomer B) | 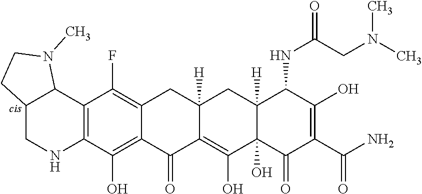 [SMILES: CN(C)CC(=O)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S4-14-1 (diastereomer A) | 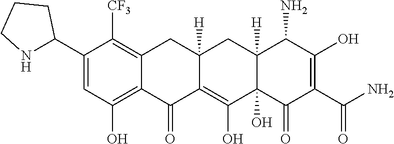 [SMILES: NC(=O)C1=C(O)[C@@H](N)[C@@H]2C[C@@H]3Cc4c(c(O)cc(C5CCCN5)c4C(F)(F)F)C(=O)C3=C(O)[C@]2(O)C1=O] |
| S4-14-2 (diastereomer A) | 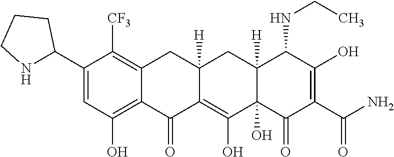 [SMILES: CCN[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-3 (diastereomer A) | 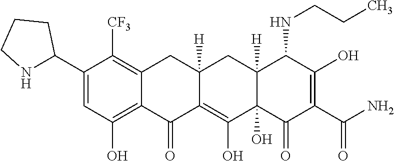 [SMILES: CCCN[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-4 (diastereomer A) | 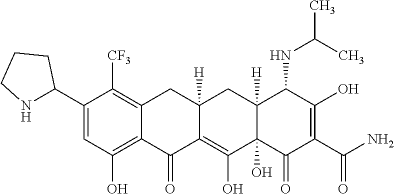 [SMILES: CC(C)N[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-5-A (diastereomer A) S4-14-5-B (diastereomer B) | 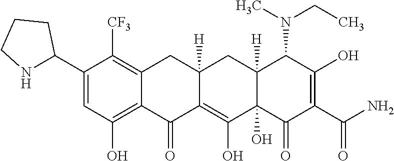 [SMILES: CCN(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-7 (diastereomer A) | 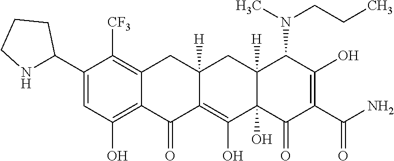 [SMILES: CCCN(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-8 (diastereomer A) | 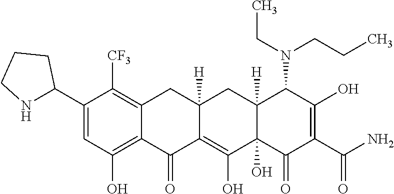 [SMILES: CCCN(CC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-9 (diastereomer A) | 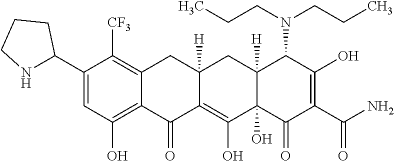 [SMILES: CCCN(CCC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-10 (diastereomer A) | 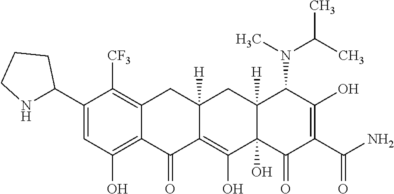 [SMILES: CC(C)N(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-11 (diastereomer A) | 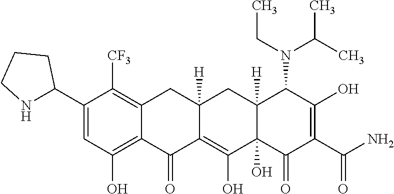 [SMILES: CCN(C(C)C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-12 (diastereomer A) | 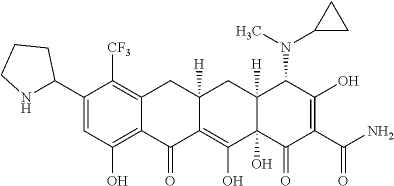 [SMILES: CN(C1CC1)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-13 (diastereomer A) | 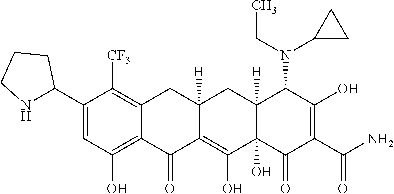 [SMILES: CCN(C1CC1)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-14-A (diastereomer A) S4-14-14-B (diastereomer B) | 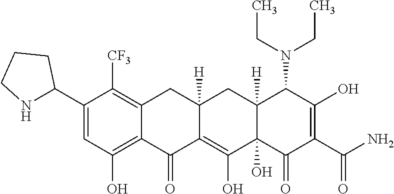 [SMILES: CCN(CC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-16 (diastereomer A) | 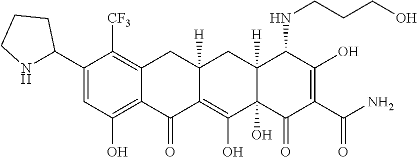 [SMILES: NC(=O)C1=C(O)[C@@H](NCCCO)[C@@H]2C[C@@H]3Cc4c(c(O)cc(C5CCCN5)c4C(F)(F)F)C(=O)C3=C(O)[C@]2(O)C1=O] |
| S4-14-17 (diastereomer A) | 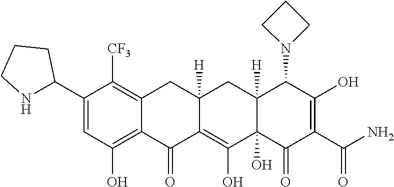 [SMILES: NC(=O)C1=C(O)[C@@H](N2CCC2)[C@@H]2C[C@@H]3Cc4c(c(O)cc(C5CCCN5)c4C(F)(F)F)C(=O)C3=C(O)[C@]2(O)C1=O] |
| S4-14-18 (diastereomer A) | 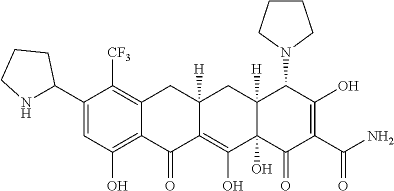 [SMILES: NC(=O)C1=C(O)[C@@H](N2CCCC2)[C@@H]2C[C@@H]3Cc4c(c(O)cc(C5CCCN5)c4C(F)(F)F)C(=O)C3=C(O)[C@]2(O)C1=O] |
| S5-10-1-A (diastereomer A) S5-10-1-B (diastereomer B) | 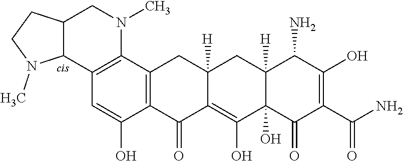 [SMILES: CN1CC2CCN(C)C2c2cc(O)c3c(c21)C[C@H]1C[C@H]2[C@H](N)C(O)=C(C(N)=O)C(=O)[C@@]2(O)C(O)=C1C3=O] |
| S5-10-1-2-A (diastereomer A) S5-10-1-2-B (diastereomer B) | 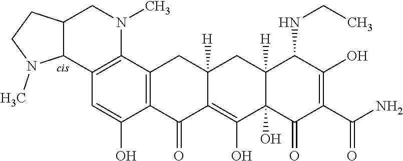 [SMILES: CCN[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S5-10-3-A (diastereomer A) S5-10-3-B (diastereomer B) | 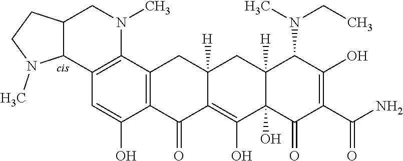 [SMILES: CCN(C)[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S5-10-4-A (diastereomer A) S5-10-4-B (diastereomer B) | 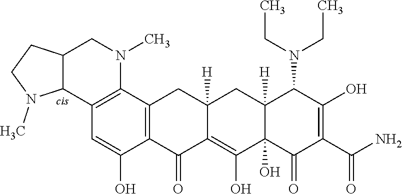 [SMILES: CCN(CC)[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S6-6-1 (single diastereomer) | 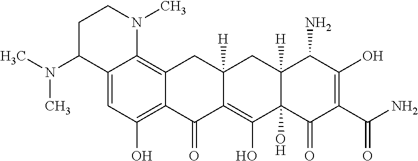 [SMILES: CN1CCC(N(C)C)c2cc(O)c3c(c21)C[C@H]1C[C@H]2[C@H](N)C(O)=C(C(N)=O)C(=O)[C@@]2(O)C(O)=C1C3=O] |
| S6-6-2 (single diastereomer) | 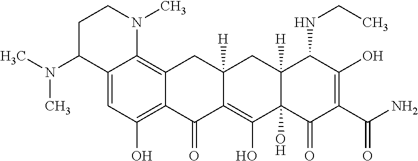 [SMILES: CCN[C@@H]1C(O)=C(C(N)=O)C(=O)[C@@]2(O)C(O)=C3C(=O)c4c(O)cc5c(c4C[C@H]3C[C@@H]12)N(C)CCC5N(C)C] |
| S6-6-3 (single diastereomer) | 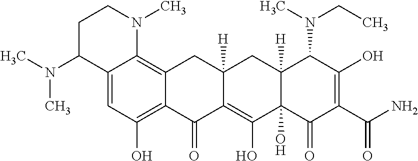 [SMILES: CCN(C)[C@@H]1C(O)=C(C(N)=O)C(=O)[C@@]2(O)C(O)=C3C(=O)c4c(O)cc5c(c4C[C@H]3C[C@@H]12)N(C)CCC5N(C)C] |
| S7-14-1-A (diastereomer A) S7-14-1-B (diastereomer B) | 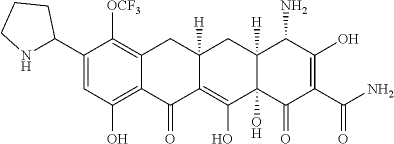 [SMILES: NC(=O)C1=C(O)[C@@H](N)[C@@H]2C[C@@H]3Cc4c(OC(F)(F)F)c(C5CCCN5)cc(O)c4C(=O)C3=C(O)[C@]2(O)C1=O] |
| S7-14-2-A (diastereomer A) | 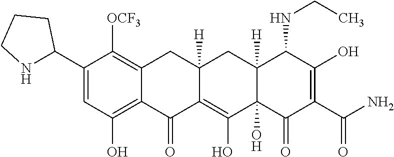 [SMILES: CCN[C@@H]1C(O)=C(C(N)=O)C(=O)[C@@]2(O)C(O)=C3C(=O)c4c(O)cc(C5CCCN5)c(OC(F)(F)F)c4C[C@H]3C[C@@H]12] |
| S7-14-3-A (diastereomer A) | 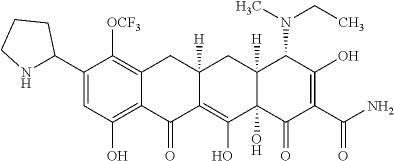 [SMILES: CCN(C)[C@@H]1C(O)=C(C(N)=O)C(=O)[C@@]2(O)C(O)=C3C(=O)c4c(O)cc(C5CCCN5)c(OC(F)(F)F)c4C[C@H]3C[C@@H]12] |
| S8-4-1 | 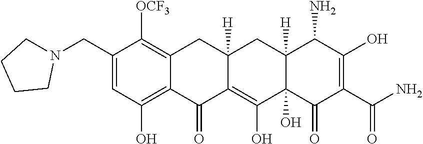 [SMILES: NC(=O)C1=C(O)[C@@H](N)[C@@H]2C[C@@H]3Cc4c(OC(F)(F)F)c(CN5CCCC5)cc(O)c4C(=O)C3=C(O)[C@]2(O)C1=O] |
| S8-4-2 | 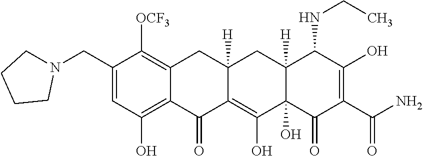 [SMILES: CCN[C@@H]1C(O)=C(C(N)=O)C(=O)[C@@]2(O)C(O)=C3C(=O)c4c(O)cc(CN5CCCC5)c(OC(F)(F)F)c4C[C@H]3C[C@@H]12] |
| S8-4-3 | 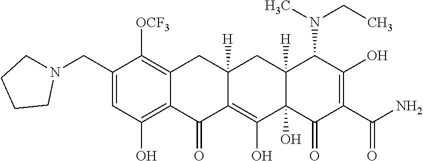 [SMILES: CCN(C)[C@@H]1C(O)=C(C(N)=O)C(=O)[C@@]2(O)C(O)=C3C(=O)c4c(O)cc(CN5CCCC5)c(OC(F)(F)F)c4C[C@H]3C[C@@H]12] |
| S9-4-1 | 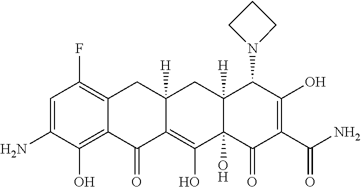 [SMILES: NC(=O)C1=C(O)[C@@H](N2CCC2)[C@@H]2C[C@@H]3Cc4c(F)cc(N)c(O)c4C(=O)C3=C(O)[C@]2(O)C1=O] |
| S9-5-1 | 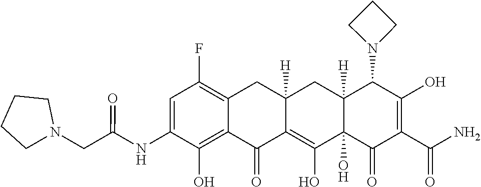 [SMILES: NC(=O)C1=C(O)[C@@H](N2CCC2)[C@@H]2C[C@@H]3Cc4c(F)cc(NC(=O)CN5CCCC5)c(O)c4C(=O)C3=C(O)[C@]2(O)C1=O] |
| S9-5-2 | 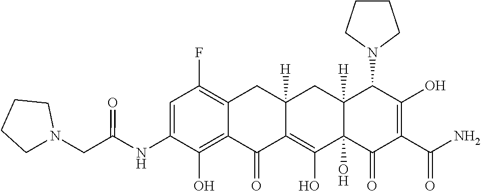 [SMILES: NC(=O)C1=C(O)[C@@H](N2CCCC2)[C@@H]2C[C@@H]3Cc4c(F)cc(NC(=O)CN5CCCC5)c(O)c4C(=O)C3=C(O)[C@]2(O)C1=O] |
| S9-5-3 | 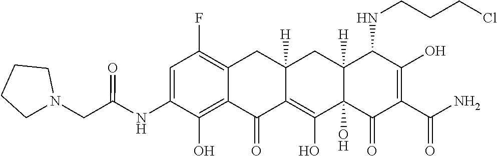 [SMILES: NC(=O)C1=C(O)[C@@H](NCCCCl)[C@@H]2C[C@@H]3Cc4c(F)cc(NC(=O)CN5CCCC5)c(O)c4C(=O)C3=C(O)[C@]2(O)C1=O] |
| S9-5-4 | 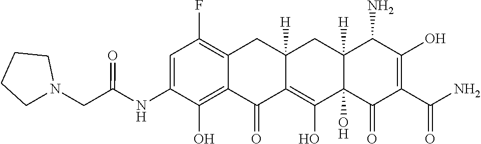 [SMILES: NC(=O)C1=C(O)[C@@H](N)[C@@H]2C[C@@H]3Cc4c(F)cc(NC(=O)CN5CCCC5)c(O)c4C(=O)C3=C(O)[C@]2(O)C1=O] |
| S9-5-5 | 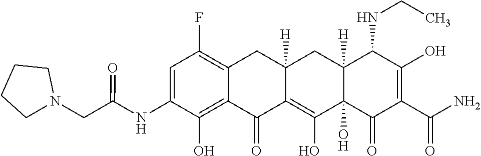 [SMILES: CCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12] |
| S9-5-6 | 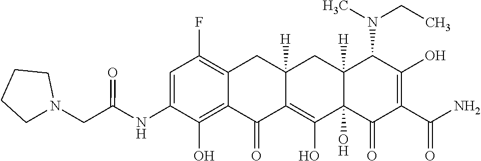 [SMILES: CCN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12] |
| S10-4-1 (single diastereomer) | 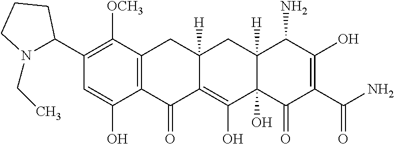 [SMILES: CCN1CCCC1c1cc(O)c2c(c1OC)C[C@H]1C[C@H]3[C@H](N)C(O)=C(C(N)=O)C(=O)[C@@]3(O)C(O)=C1C2=O] |
| S10-4-2 (single diastereomer) | 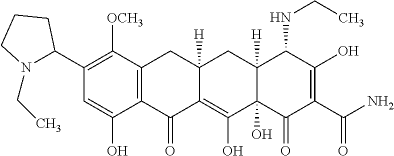 [SMILES: CCN[C@@H]1C(O)=C(C(N)=O)C(=O)[C@@]2(O)C(O)=C3C(=O)c4c(O)cc(C5CCCN5CC)c(OC)c4C[C@H]3C[C@@H]12] |
| S10-4-3 (single diastereomer) | 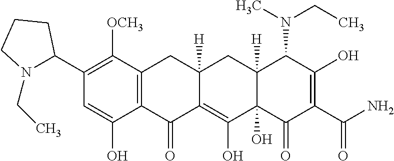 [SMILES: CCN1CCCC1c1cc(O)c2c(c1OC)C[C@H]1C[C@H]3[C@H](N(C)CC)C(O)=C(C(N)=O)C(=O)[C@@]3(O)C(O)=C1C2=O] |
| S11-3-1 | 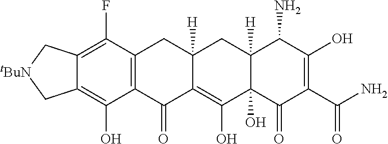 |
| S11-3-2 | 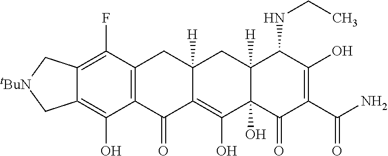 |
| S11-3-3 | 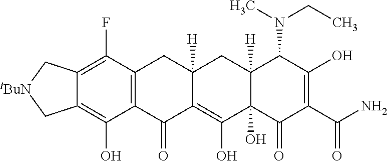 |
| S12-8-1-A (diastereomer A) S12-8-1-B (diastereomer B) | 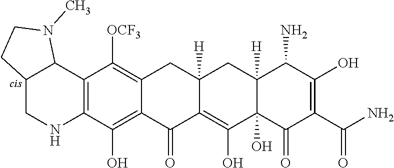 [SMILES: CN1CCC2CNc3c(O)c4c(c(OC(F)(F)F)c3C21)C[C@H]1C[C@H]2[C@H](N)C(O)=C(C(N)=O)C(=O)[C@@]2(O)C(O)=C1C4=O] |
| S12-8-2-A (diastereomer A) | 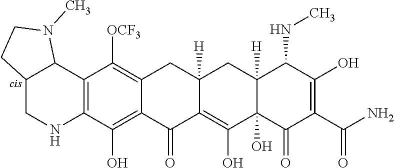 [SMILES: CN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-3-A (diastereomer A) S12-8-3-B) (diastereomer B) | 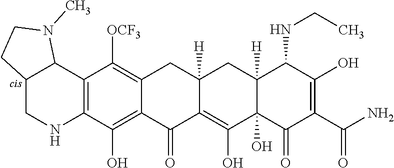 [SMILES: CCN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-4-A (diastereomer A) | 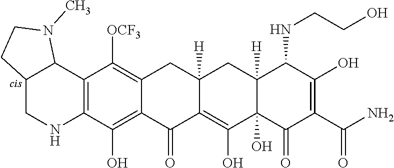 [SMILES: CN1CCC2CNc3c(O)c4c(c(OC(F)(F)F)c3C21)C[C@H]1C[C@H]2[C@H](NCCO)C(O)=C(C(N)=O)C(=O)[C@@]2(O)C(O)=C1C4=O] |
| S12-8-5-A (diastereomer A) | 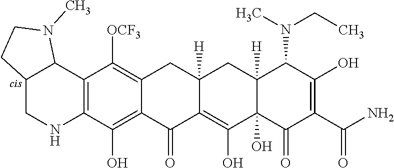 [SMILES: CCN(C)[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-6-A (diastereomer A) S12-8-6-B (diastereomer B) | 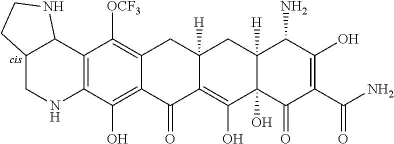 [SMILES: NC(=O)C1=C(O)[C@@H](N)[C@@H]2C[C@@H]3Cc4c(OC(F)(F)F)c5c(c(O)c4C(=O)C3=C(O)[C@]2(O)C1=O)NCC1CCNC51] |
| S12-8-7-A (diastereomer A) | 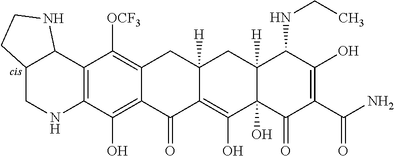 [SMILES: CCN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1NCCC1CN5] |
| S12-8-8-A (diastereomer A) | 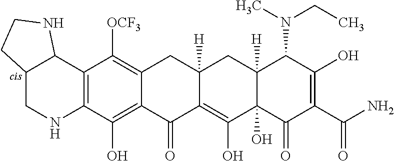 [SMILES: CCN(C)[C@@H]1C(O)=C(C(N)=O)C(=O)[C@@]2(O)C(O)=C3C(=O)c4c(O)c5c(c(OC(F)(F)F)c4C[C@H]3C[C@@H]12)C1NCCC1CN5] |
| S13-5-1 | 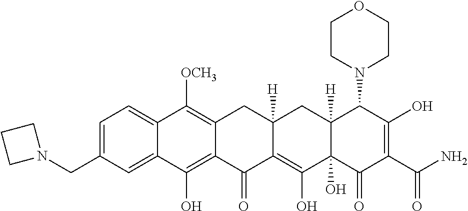 [SMILES: COc1c2c(c(O)c3cc(CN4CCC4)ccc13)C(=O)C1=C(O)[C@]3(O)C(=O)C(C(N)=O)=C(O)[C@@H](N4CCOCC4)[C@@H]3C[C@@H]1C2] |
| S13-5-2 | 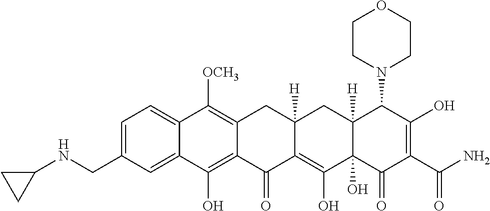 [SMILES: COc1c2c(c(O)c3cc(CNC4CC4)ccc13)C(=O)C1=C(O)[C@]3(O)C(=O)C(C(N)=O)=C(O)[C@@H](N4CCOCC4)[C@@H]3C[C@@H]1C2] |
| S14-8-1 | 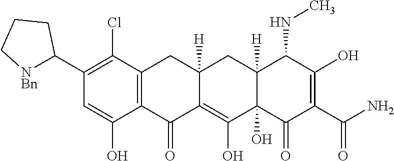 [SMILES: CN[C@@H]1C(O)=C(C(N)=O)C(=O)[C@@]2(O)C(O)=C3C(=O)c4c(O)cc(C5CCCN5Cc5ccccc5)c(Cl)c4C[C@H]3C[C@@H]12] |
| S14-8-2 | 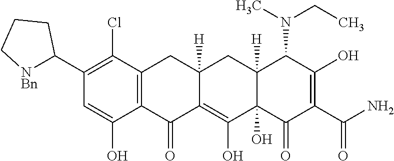 [SMILES: CCN(C)[C@@H]1C(O)=C(C(N)=O)C(=O)[C@@]2(O)C(O)=C3C(=O)c4c(O)cc(C5CCCN5Cc5ccccc5)c(Cl)c4C[C@H]3C[C@@H]12] |
| S14-8-3-A (diastereomer A) S14-8-3-B (diastereomer B) | 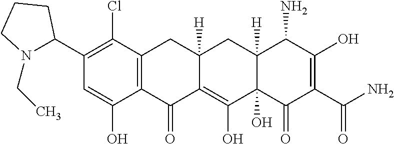 [SMILES: CCN1CCCC1c1cc(O)c2c(c1Cl)C[C@H]1C[C@H]3[C@H](N)C(O)=C(C(N)=O)C(=O)[C@@]3(O)C(O)=C1C2=O] |
| S15-10-1 | 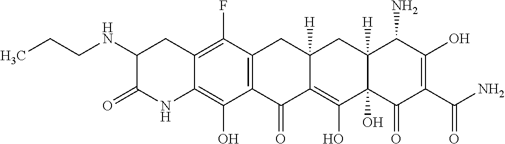 [SMILES: CCCNC1Cc2c(F)c3c(c(O)c2NC1=O)C(=O)C1=C(O)[C@]2(O)C(=O)C(C(N)=O)=C(O)[C@@H](N)[C@@H]2C[C@@H]1C3] |
| S15-10-2 | 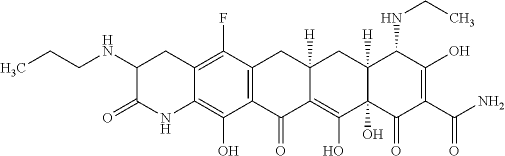 [SMILES: CCCNC1Cc2c(F)c3c(c(O)c2NC1=O)C(=O)C1=C(O)[C@]2(O)C(=O)C(C(N)=O)=C(O)[C@@H](NCC)[C@@H]2C[C@@H]1C3] |
| S15-10-3-A (diastereomer A) S15-10-3-B (diastereomer B) | 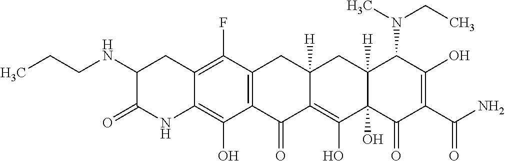 [SMILES: CCCNC1Cc2c(F)c3c(c(O)c2NC1=O)C(=O)C1=C(O)[C@]2(O)C(=O)C(C(N)=O)=C(O)[C@@H](N(C)CC)[C@@H]2C[C@@H]1C3] |
| S16-7-1 (single diastereomer) | 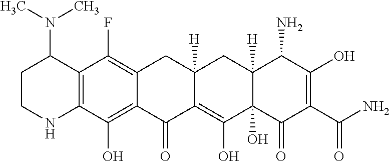 [SMILES: CN(C)C1CCNc2c(O)c3c(c(F)c21)C[C@H]1C[C@H]2[C@H](N)C(O)=C(C(N)=O)C(=O)[C@@]2(O)C(O)=C1C3=O] |
| S16-7-2 (single diastereomer) | 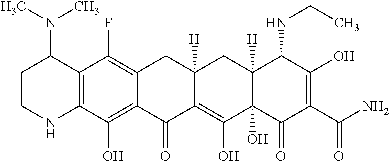 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S16-7-3 (single diastereomer) | 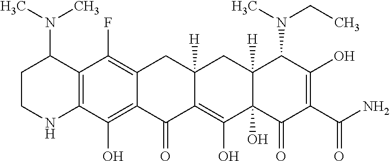 [SMILES: CCN(C)[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S16-7-4 (single diastereomer) | 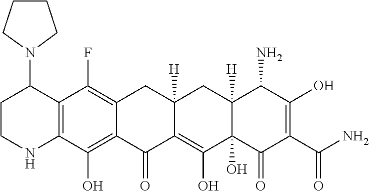 [SMILES: NC(=O)C1=C(O)[C@@H](N)[C@@H]2C[C@@H]3Cc4c(F)c5c(c(O)c4C(=O)C3=C(O)[C@]2(O)C1=O)NCCC5N1CCCC1] |
| S16-7-5 (single diastereomer) | 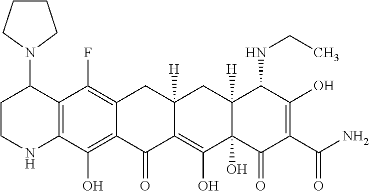 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C(N1CCCC1)CCN5] |
| S16-7-6 (single diastereomer) | 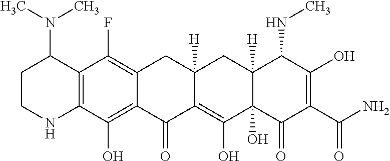 [SMILES: CN[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S17-3-1 | 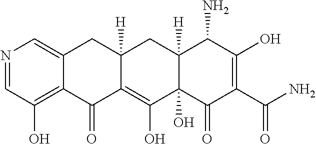 [SMILES: NC(=O)C1=C(O)[C@@H](N)[C@@H]2C[C@@H]3Cc4cncc(O)c4C(=O)C3=C(O)[C@]2(O)C1=O] |
| S17-3-2 | 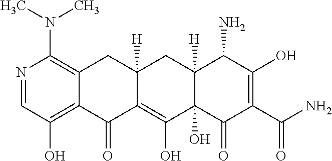 [SMILES: CN(C)c1ncc(O)c2c1C[C@H]1C[C@H]3[C@H](N)C(O)=C(C(N)=O)C(=O)[C@@]3(O)C(O)=C1C2=O] |
| S17-3-3 | 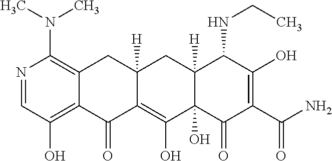 [SMILES: CCN[C@@H]1C(O)=C(C(N)=O)C(=O)[C@@]2(O)C(O)=C3C(=O)c4c(O)cnc(N(C)C)c4C[C@H]3C[C@@H]12] |
| S17-3-4 | 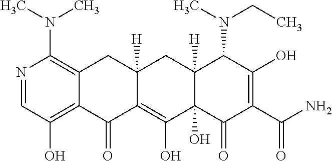 [SMILES: CCN(C)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-5 | 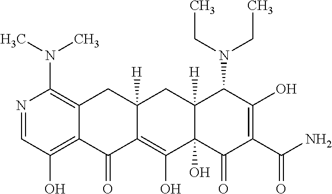 [SMILES: CCN(CC)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-6 | 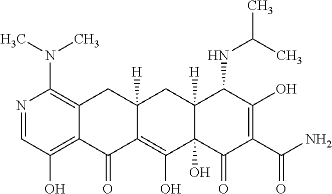 [SMILES: CC(C)N[C@@H]1C(O)=C(C(N)=O)C(=O)[C@@]2(O)C(O)=C3C(=O)c4c(O)cnc(N(C)C)c4C[C@H]3C[C@@H]12] |
| S17-3-7 | 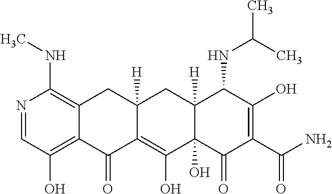 [SMILES: CNc1ncc(O)c2c1C[C@H]1C[C@H]3[C@H](NC(C)C)C(O)=C(C(N)=O)C(=O)[C@@]3(O)C(O)=C1C2=O] |
| S17-3-8 | 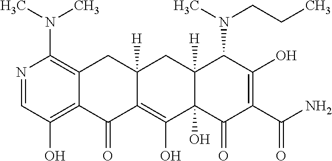 [SMILES: CCCN(C)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-9 | 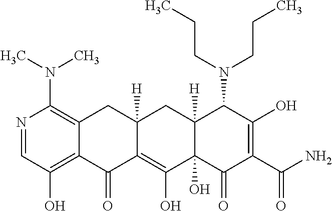 [SMILES: CCCN(CCC)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-10 | 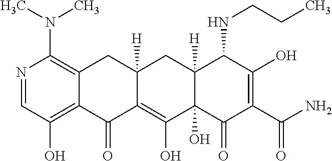 [SMILES: CCCN[C@@H]1C(O)=C(C(N)=O)C(=O)[C@@]2(O)C(O)=C3C(=O)c4c(O)cnc(N(C)C)c4C[C@H]3C[C@@H]12] |
| S17-3-11 | 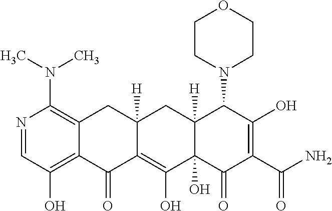 [SMILES: CN(C)c1ncc(O)c2c1C[C@H]1C[C@H]3[C@H](N4CCOCC4)C(O)=C(C(N)=O)C(=O)[C@@]3(O)C(O)=C1C2=O] |
| S18-5-1-1 | 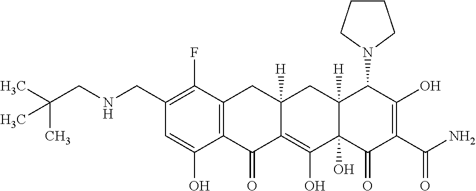 [SMILES: CC(C)(C)CNCc1cc(O)c2c(c1F)C[C@H]1C[C@H]3[C@H](N4CCCC4)C(O)=C(C(N)=O)C(=O)[C@@]3(O)C(O)=C1C2=O] |
| S18-5-1-2 | 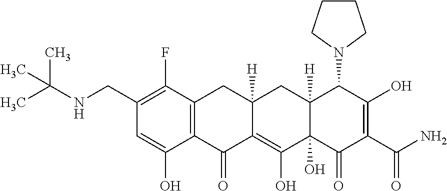 [SMILES: CC(C)(C)NCc1cc(O)c2c(c1F)C[C@H]1C[C@H]3[C@H](N4CCCC4)C(O)=C(C(N)=O)C(=O)[C@@]3(O)C(O)=C1C2=O] |
| S18-5-2-1 | 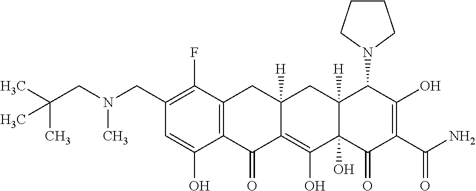 [SMILES: CN(Cc1cc(O)c2c(c1F)C[C@H]1C[C@H]3[C@H](N4CCCC4)C(O)=C(C(N)=O)C(=O)[C@@]3(O)C(O)=C1C2=O)CC(C)(C)C] |
| S18-5-2-2 | 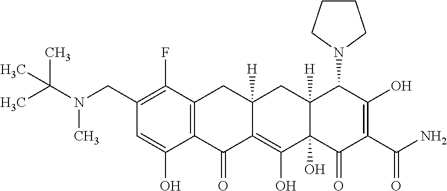 [SMILES: CN(Cc1cc(O)c2c(c1F)C[C@H]1C[C@H]3[C@H](N4CCCC4)C(O)=C(C(N)=O)C(=O)[C@@]3(O)C(O)=C1C2=O)C(C)(C)C] |
| S19-7-1-B (diastereomer B) | 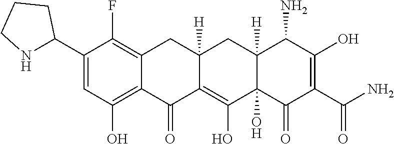 [SMILES: NC(=O)C1=C(O)[C@@H](N)[C@@H]2C[C@@H]3Cc4c(F)c(C5CCCN5)cc(O)c4C(=O)C3=C(O)[C@]2(O)C1=O] |
| S19-7-2 | 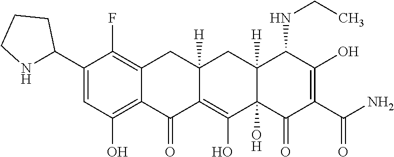 [SMILES: CCN[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-3-A (diastereomer A) S19-7-3-B (diastereomer B) | 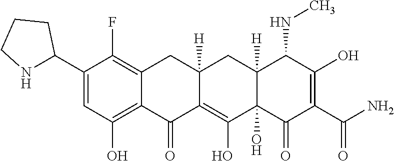 [SMILES: CN[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-4-A (diastereomer A) S19-7-4-B (diastereomer B) | 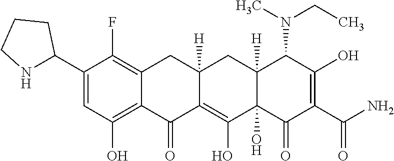 [SMILES: CCN(C)[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-5-A (diastereomer A) S19-7-5-B (diastereomer B) | 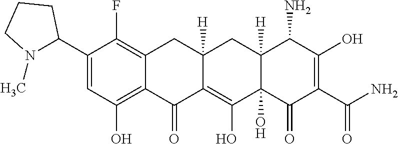 [SMILES: CN1CCCC1c1cc(O)c2c(c1F)C[C@H]1C[C@H]3[C@H](N)C(O)=C(C(N)=O)C(=O)[C@@]3(O)C(O)=C1C2=O] |
| S19-7-6 | 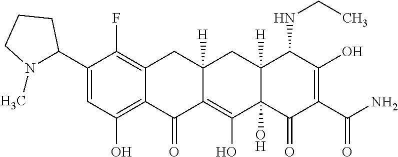 [SMILES: CCN[C@@H]1C(O)=C(C(N)=O)C(=O)[C@@]2(O)C(O)=C3C(=O)c4c(O)cc(C5CCCN5C)c(F)c4C[C@H]3C[C@@H]12] |
| S19-7-7-A (diastereomer A) S19-7-7-B (diastereomer B) | 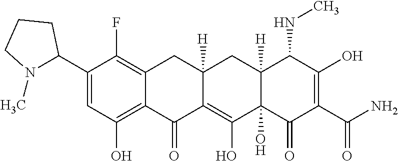 [SMILES: CN[C@@H]1C(O)=C(C(N)=O)C(=O)[C@@]2(O)C(O)=C3C(=O)c4c(O)cc(C5CCCN5C)c(F)c4C[C@H]3C[C@@H]12] |
| S20-4-1 (single diastereomer) | 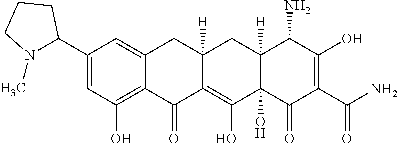 [SMILES: CN1CCCC1c1cc(O)c2c(c1)C[C@H]1C[C@H]3[C@H](N)C(O)=C(C(N)=O)C(=O)[C@@]3(O)C(O)=C1C2=O] |
| S20-4-2 (single diastereomer) | 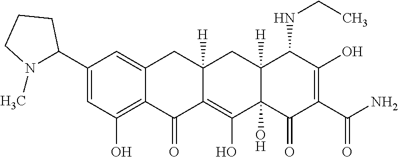 [SMILES: CCN[C@@H]1C(O)=C(C(N)=O)C(=O)[C@@]2(O)C(O)=C3C(=O)c4c(O)cc(C5CCCN5C)cc4C[C@H]3C[C@@H]12] |
| S20-4-3 (single diastereomer) | 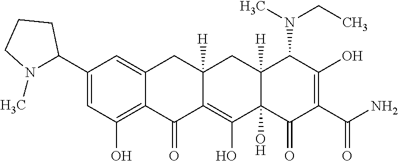 [SMILES: CCN(C)[C@@H]1C(O)=C(C(N)=O)C(=O)[C@@]2(O)C(O)=C3C(=O)c4c(O)cc(C5CCCN5C)cc4C[C@H]3C[C@@H]12] |
| S20-4-4 (single diastereomer) | 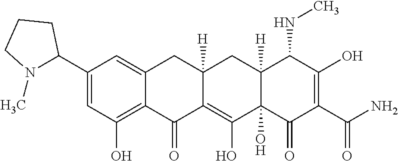 [SMILES: CN[C@@H]1C(O)=C(C(N)=O)C(=O)[C@@]2(O)C(O)=C3C(=O)c4c(O)cc(C5CCCN5C)cc4C[C@H]3C[C@@H]12] |
| S21-5-1 | 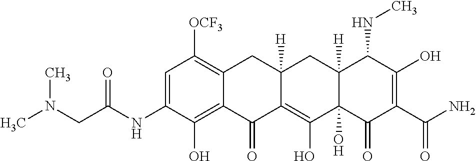 [SMILES: CN[C@@H]1C(O)=C(C(N)=O)C(=O)[C@@]2(O)C(O)=C3C(=O)c4c(O)c(NC(=O)CN(C)C)cc(OC(F)(F)F)c4C[C@H]3C[C@@H]12] |
| S21-5-2 | 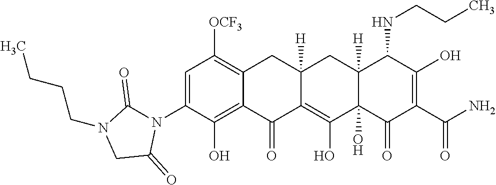 [SMILES: CCCCN1CC(=O)N(c2cc(OC(F)(F)F)c3c(c2O)C(=O)C2=C(O)[C@]4(O)C(=O)C(C(N)=O)=C(O)[C@@H](NCCC)[C@@H]4C[C@@H]2C3)C1=O] |
| S21-5-3 | 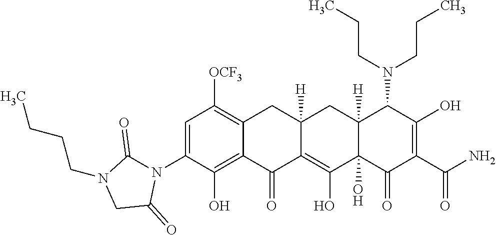 [SMILES: CCCCN1CC(=O)N(c2cc(OC(F)(F)F)c3c(c2O)C(=O)C2=C(O)[C@]4(O)C(=O)C(C(N)=O)=C(O)[C@@H](N(CCC)CCC)[C@@H]4C[C@@H]2C3)C1=O] |
| S21-5-4 | 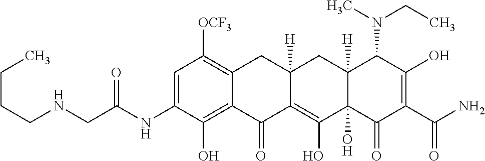 [SMILES: CCCCNCC(=O)Nc1cc(OC(F)(F)F)c2c(c1O)C(=O)C1=C(O)[C@]3(O)C(=O)C(C(N)=O)=C(O)[C@@H](N(C)CC)[C@@H]3C[C@@H]1C2] |

The compounds set forth in the above tables were prepared according to the synthetic procedures described in WO2014/036502, incorporated herein by reference in its entirety. The compound numbers in the tables set forth above reference synthetic schemes in WO2014/03650 all of which are found in U.S. Pat. No. 9,573,895 the entire content of which is hereby incorporated by reference.

In a second embodiment of the invention, the compound administered in the method of treating a hematological cancer is a compound of Structural Formula (I) or (I′), wherein R⁴ is selected from hydrogen and —(C₁-C₆ alkyl); R^{4′} is selected from hydrogen, —(C₂-C₆ alkyl) optionally substituted with one or more substituents independently selected from hydroxy and halo, —(C₃-C₆ cycloalkyl), —C(O)—(C₁-C₆ alkyl), —C(O)—(C₁-C₆ alkylene)-N(R^{D})(R^{E}), and S(O)₁₋₂R^{C}; or R⁴ and R^{4′} are taken together with the nitrogen atom to which they are commonly bound to form a 4-6 membered ring optionally comprising 1-2 additional heteroatoms independently selected from N, O and S; R^{C} is —(C₁-C₆ alkyl); and each of R^{D} and R^{E} is independently selected from hydrogen and —(C₁-C₆ alkyl). The remaining variables are as described and defined in the first embodiment, or any aspect thereof.

In a first aspect of the second embodiment, R⁴ is selected from hydrogen, methyl, ethyl and propyl; and R^{4′} is selected from hydrogen, ethyl, propyl, cyclopropyl, —C(O)CH₃, —C(O)CH₂N(CH₃)₂, and —S(O)₂CH₃. The remaining variables are as described and defined in the first embodiment, or any aspect thereof, or in the second embodiment.

In a second aspect of the second embodiment, R⁴ is selected from hydrogen and —(C₁-C₆ alkyl); R^{4′} is selected from hydrogen, —(C₂-C₆ alkyl), —(C₃-C₆ cycloalkyl), —C(O)—(C₁-C₆ alkyl), —C(O)—(C₁-C₆ alkylene)-N(R^{D})(R^{E}), and S(O)₁₋₂R^{C}; R^{C} is —(C₁-C₆ alkyl); and each of R^{D} and R^{E} is independently selected from hydrogen and —(C₁-C₆ alkyl). The remaining variables are as described and defined in the first embodiment, or any aspect thereof, or the second embodiment, or first aspect thereof.

In a third aspect of the second embodiment, R⁴ and R^{4′} are both hydrogen.

In a fourth aspect of the second embodiment, R⁴ is —(C₁-C₆ alkyl) and R^{4′} is —(C₂-C₆ alkyl).

In a fifth aspect of the second embodiment, R⁴ is hydrogen and R^{4′} is —(C₂-C₆ alkyl).

In a third embodiment of the invention, the compound administered in the method of treating a hematological cancer is a compound of Structural Formula (I) or (I′), wherein R¹ is selected from hydrogen, halo, and —(C₁-C₆ alkyl) optionally substituted with one or more substituents independently selected from halo, —NR^{B}R^{B′}, —C(O)NR^{B}R^{B′}, —OR^{A}, —(C₀-C₆ alkylene)-carbocyclyl, and —(C₀-C₆ alkylene)-heterocyclyl, wherein R^{A} is C₁-C₆ alkyl optionally substituted with one or more fluoro. The remaining variables are as described and defined in the first or second embodiment, or any aspect thereof.

In a first aspect of the third embodiment, X is C(R²). The remaining variables are as described and defined in the first or second embodiment, or any aspect thereof, or the third embodiment.

In a second aspect of the third embodiment, R¹ is selected from hydrogen, fluoro, chloro, CF₃ and OCF₃. The remaining variables are as described and defined in the first or second embodiment, or any aspect thereof, or the third embodiment, or first aspect thereof.

In a third aspect of the third embodiment, R¹ is selected from hydrogen, halo, and —(C₁-C₆ alkyl) optionally substituted with one or more substituents independently selected from halo, and —OR^{A}, wherein R^{A} is C₁-C₆ alkyl optionally substituted with one or more fluoro. The remaining variables are as described and defined in the first or second embodiment, or any aspect thereof, or the third embodiment, or first or second aspect thereof.

In a fourth aspect of the third embodiment, R¹ is selected from hydrogen, fluoro, chloro, —CF₃, —OCH₃, —OCF₃, —N(CH₃)₂ and —NHCH₃. The remaining variables are as described and defined in the first or second embodiment, or any aspect thereof, or the third embodiment, or first, second or third aspect thereof.

In a fourth embodiment of the invention, the compound administered in the method of treating a hematological cancer is a compound of Structural Formula (I) or (I′), wherein R¹ and R² are taken together with the atoms to which they are bound to form a nitrogen-containing heterocyclyl ring, wherein the ring comprising R¹ and R² is optionally substituted on any substitutable nitrogen atom with C₁-C₄ alkyl; and optionally substituted on a carbon atom with NR^{B}R^{B′}, wherein each of R^{B} and R^{B′} is independently selected from hydrogen and C₁-C₆ alkyl. The remaining variables are as described and defined in the first, second or third embodiment, or any aspect thereof.

In a first aspect of the fourth embodiment, R¹ and R² are taken together with the carbon atoms to which they are bound to form:

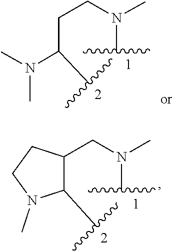

wherein “ [symbol] 1” represents a point of attachment to the carbon atom bound to R¹ and “ [symbol] 2” represents a point of attachment to the carbon atom bound to R². The remaining variables are as described and defined in the first, second or third embodiment, or any aspect thereof, or the fourth embodiment.

In a second aspect of the fourth embodiment, X is C(R²). The remaining variables are as described and defined in the first, second or third embodiment, or any aspect thereof, or the fourth embodiment, or the first aspect thereof.

In a third aspect of the fourth embodiment, X is C(R²); and R¹ and R² are taken together with the carbon atoms to which they are bound to form:

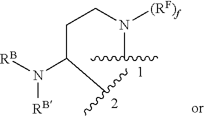

wherein “ [symbol] 1” represents a point of attachment to the carbon atom bound to R¹; “ [symbol] 2” represents a point of attachment to the carbon atom bound to R²; and f is 0 or 1. The remaining variables are as described and defined in the first, second or third embodiment, or any aspect thereof, or the fourth embodiment, or the first or second aspect thereof.

In a fifth embodiment of the invention, the compound administered in the method of treating a hematological cancer is a compound of Structural Formula (I) or (I′), wherein R² is —(C₀-C₆ alkylene)-heterocyclyl optionally substituted on a nitrogen atom, if present, with —(C₁-C₆ alkyl); —(C₀-C₆ alkylene)-carbocyclyl; or —(C₁-C₆)alkyl substituted with NR^{B}R^{B′}. The remaining variables are as described and defined in the first, second, third or fourth embodiment, or any aspect thereof.

In a first aspect of the fifth embodiment, R² is pyrrolidinyl optionally substituted on a nitrogen atom with C₁-C₄ alkyl or benzyl. The remaining variables are as described and defined in the first, second, third or fourth embodiment, or any aspect thereof, or the fifth embodiment.

In a third aspect of the fifth embodiment, R² is —(C₀-C₆ alkylene)-heterocyclyl optionally substituted on a nitrogen atom, if present, with —(C₁-C₆ alkyl) or —(C₀-C₆ alkylene)-carbocyclyl. The remaining variables are as described and defined in the first, second, third or fourth embodiment, or any aspect thereof, or the fifth embodiment, or first or second aspect thereof.

In a sixth embodiment of the invention, the compound administered in the method of treating a hematological cancer is a compound of Structural Formula (I) or (I′), wherein R² and R³ are taken together with the atoms to which they are bound to form a heterocyclyl, e.g., a nitrogen-containing heterocyclyl ring, wherein the ring comprising R² and R³ is optionally and independently substituted on any substitutable nitrogen atom with C₁-C₄ alkyl. The remaining variables are as described and defined in the first, second, third, fourth or fifth embodiment, or any aspect thereof.

In a first aspect of the sixth embodiment, R² and R³ are taken together with the atoms to which they are bound to form

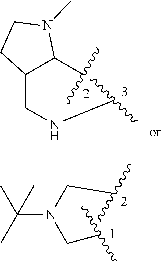

or wherein “ [symbol] 2” represents a point of attachment to the carbon atom bound to R², and “ [symbol] 3” represents a point of attachment to the carbon atom bound to R³. The remaining variables are as described and defined in the first, second, third, fourth or fifth embodiment, or any aspect thereof, or the sixth embodiment.

In a second aspect of the sixth embodiment, R² and R³ are taken together with the atoms to which they are bound to form

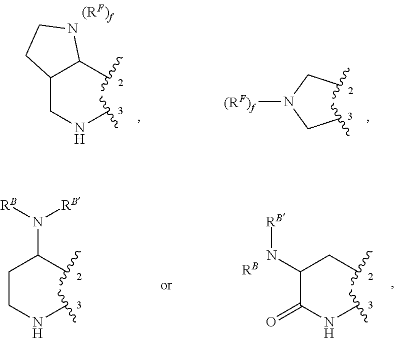

wherein “ [symbol] 2” represents a point of attachment to the carbon atom bound to R²; “ [symbol] 3” represents a point of attachment to the carbon atom bound to R³; and f is 0 or 1. The remaining variables are as described and defined in the first, second, third, fourth or fifth embodiment, or any aspect thereof, or the sixth embodiment, or first aspect thereof.

In a seventh embodiment of the invention, the compound administered in the method of treating a hematological cancer is a compound of Structural Formula (I) or (I′), wherein R³ is selected from hydrogen and —N(R^{B})(R^{B′}), wherein R^{B} is hydrogen and R^{B′} is —C(O)—(C₀-C₆ alkylene)-heterocyclyl or —C(O)—(C₀-C₆ alkylene)-N(R^{D})(R^{E}). The remaining variables are as described and defined in the first, second, third, fourth, fifth or sixth embodiment, or any aspect thereof.

In a first aspect of the seventh embodiment, R³ is selected from hydrogen and

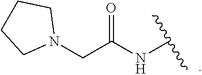 [SMILES: *NC(=O)CN1CCCC1]

The remaining variables are as described and defined in the first, second, third, fourth, fifth or sixth embodiment, or any aspect thereof, or the seventh embodiment.

In a second aspect of the seventh embodiment, X is C(R²). The remaining variables are as described and defined in the first, second, third, fourth, fifth or sixth embodiment, or any aspect thereof, or the seventh embodiment, or first aspect thereof.

In a third aspect of the seventh embodiment, R³ is selected from hydrogen and —N(R^{B})(R^{B′}), wherein R^{B} is hydrogen and R^{B′} is —C(O)—(C₀-C₆ alkylene)-heterocyclyl. The remaining variables are as described and defined in the first, second, third, fourth, fifth or sixth embodiment, or any aspect thereof, or the seventh embodiment, or first or second aspect thereof.

In an eighth embodiment of the invention, the compound administered in the method of treating a hematological cancer is a compound of Formula II:

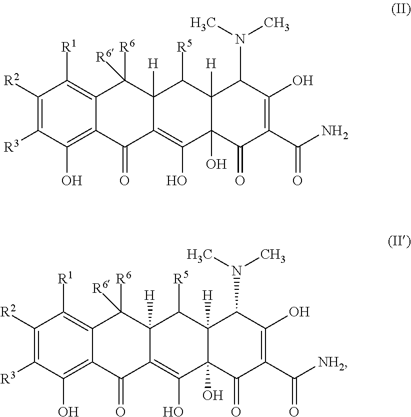

or a pharmaceutically acceptable salt thereof, wherein:

R¹ and R² are taken together with atoms to which they are bound to form a carbocyclyl or heterocyclyl ring and R³ is selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylene)-carbocyclyl, and —(C₀-C₆ alkylene)-heterocyclyl; or

R² and R³ are taken together with atoms to which they are bound to form a carbocyclyl or heterocyclyl ring and R¹ is selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylene)-carbocyclyl, and —(C₀-C₆ alkylene)-heterocyclyl;

each of R⁵ and R⁶ is independently selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylene)-carbocyclyl, and —(C₀-C₆ alkylene)-heterocyclyl;

R^{6′} is selected from hydrogen, —(C₁-C₆ alkyl) and —(C₃-C₆ cycloalkyl);

each R^{A} is independently selected from hydrogen, —(C₁-C₆ alkyl), —(C₀-C₆ alkylene)-carbocyclyl, —(C₀-C₆ alkylene)-heterocyclyl, —C(O)—(C₁-C₆ alkyl), —C(O)—(C₀-C₆ alkylene)-carbocyclyl, —C(O)—(C₀-C₆ alkylene)-heterocyclyl, and —C(O)N(R^{D})(R^{E});

each R^{B} and each R^{B′} is independently selected from hydrogen, —(C₁-C₆ alkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, and —C(O)—(C₀-C₆ alkylene)-N(R^{D})(R^{E});

each R^{C} is independently selected from —(C₁-C₆ alkyl), —(C₀-C₆ alkylene)-carbocyclyl and —(C₀-C₆ alkylene)-heterocyclyl; and

each R^{D} and each R^{E} is independently selected from hydrogen, —(C₁-C₆ alkyl), —(C₀-C₆ alkylene)-carbocyclyl, and —(C₀-C₆ alkylene)-heterocyclyl, wherein any alkyl, alkylene, carbocyclyl or heterocyclyl portion of R¹, R², R³, R⁵, R⁶, R^{6′}, R^{A}, R^{B}, R^{B′}, R^{C}, R^{D}, or R^{E} or formed by taking R¹ and R² or R² and R³ together is optionally and independently substituted.

Alternative values for the variables in Formula II are as described and defined in the first through seventh embodiments, or any aspect thereof.

In a first aspect of the eighth embodiment, the compound is represented by Formula IIa:

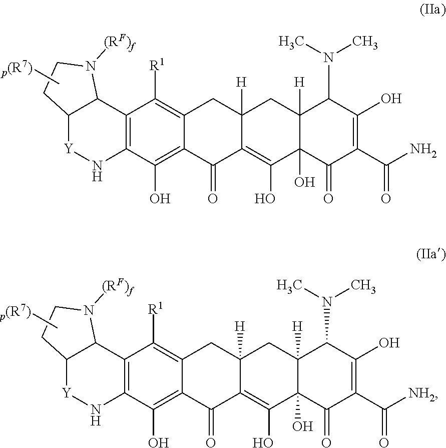

or a pharmaceutically acceptable salt thereof, wherein:

each R⁷, if present, is independently selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C};

p is 0, 1, 2, 3 or 4;

Y is C(O) or C(R⁸)₂ wherein each R⁸ is independently selected from hydrogen, —(C₁-C₆)alkyl and —(C₃-C₆ cycloalkyl); and

f is 0 or 1. The remaining variables are as described and defined in the first through seventh embodiments, or any aspect thereof, or the eighth embodiment.

In a further aspect of the first aspect of the eighth embodiment, p is 0. The remaining variables are as described and defined in the first through seventh embodiments, or any aspect thereof, or the eighth embodiment, or first aspect thereof.

In a second aspect of the eighth embodiment, the compound is represented by Formula IIb:

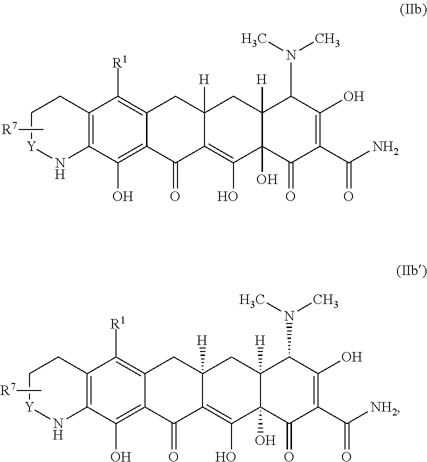

or a pharmaceutically acceptable salt thereof, wherein R⁷ is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}; and Y is C(O) or C(R⁸)₂ wherein each R⁸ is independently selected from hydrogen, —(C₁-C₆)alkyl and —(C₃-C₆ cycloalkyl). The remaining variables are as described and defined in the first through seventh embodiments, or any aspect thereof, or the eighth embodiment, or first aspect thereof.

In a third aspect of the eighth embodiment, the compound is represented by Formula IIb-1:

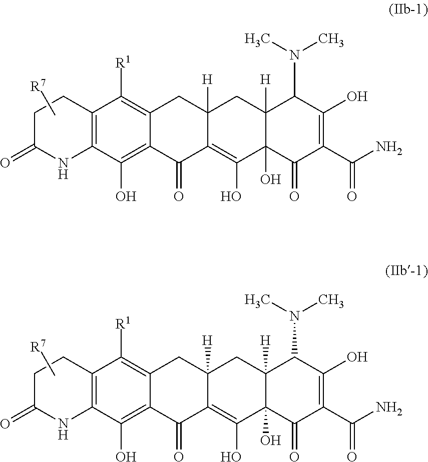

or a pharmaceutically acceptable salt thereof, wherein R⁷ is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}. The remaining variables are as described and defined in the first through seventh embodiments, or any aspect thereof, or the eighth embodiment, or first or second aspect thereof.

In a fourth aspect of the eighth embodiment, the compound is represented by Formula IId:

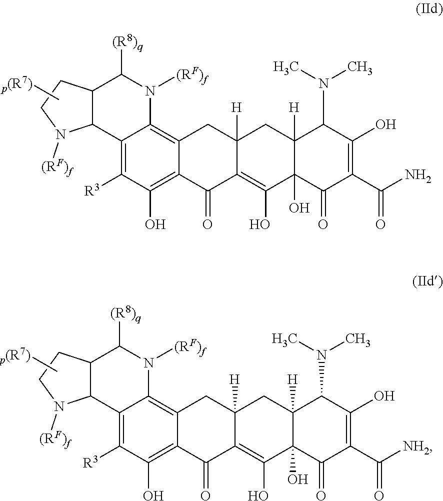

or a pharmaceutically acceptable salt thereof, wherein:

each R⁷ and R⁸, if present, is independently selected from halo, ═O, C₁-C₄ fluoroalkyl, C₁-C₄ alkyl, C₃-C₁₀ carbocyclyl, a 4-13 membered heterocyclyl, OR^{A}, —(C₀-C₆ alkylene)-NR^{B}R^{B′}, and S(O)₀₋₂R^{C};

p is 0, 1, 2, 3 or 4;

q is 0, 1 or 2; and

each f is independently 0 or 1. The remaining variables are as described and defined in the first through seventh embodiments, or any aspect thereof, or the eighth embodiment, or first through third aspects thereof.

In a further aspect of the fourth aspect of the eighth embodiment, p and q are each 0. The remaining variables are as described and defined in the first through seventh embodiments, or any aspect thereof, or the eighth embodiment, or first through fourth aspects thereof.

In a fifth aspect of the eighth embodiment, each R^{F} is independently selected from —(C₁-C₆ alkyl), —(C₁-C₆ haloalkyl), —(C₁-C₆ hydroxyalkyl), —(C₀-C₆ alkylene)-carbocyclyl, —(C₀-C₆ alkylene)-heterocyclyl, —(C₀-C₆ alkylene)-C(O)₂—(C₁-C₆ alkyl) and —(C₁-C₆ alkylene)-NR^{B}R^{B′}. The remaining variables are as described and defined in the first through seventh embodiments, or any aspect thereof, or the eighth embodiment, or first through fourth aspects thereof.

In a sixth aspect of the eighth embodiment, each f is 0. The remaining variables are as described and defined in the first through seventh embodiments, or any aspect thereof, or the eighth embodiment, or first through fifth aspects thereof.

In a seventh aspect of the eighth embodiment, each f is 1. The remaining variables are as described and defined in the first through seventh embodiments, or any aspect thereof, or the eighth embodiment, or first through sixth aspects thereof.

In an eighth aspect of the eighth embodiment, the ring formed by R¹ and R² or R² and R³ together with atoms to which they are bound is a 4-7 membered non-aromatic heterocyclic ring optionally containing 1-2 heteroatoms independently selected from N, S and O. The remaining variables are as described and defined in the first through seventh embodiments, or any aspect thereof, or the eighth embodiment, or first through seventh aspects thereof.

In a ninth aspect of the eighth embodiment:
any alkyl, or alkylene portion of R¹, R², R³, R⁵, R⁶ is optionally and independently substituted with one or more substituents independently selected from halo, ═O, OR^{A} NR^{B}R^{B′}, and S(O)₀₋₂R^{C};
any alkyl or alkylene portion of R^{6′}, R^{A}, or R^{C}, is optionally and independently substituted with one or more fluoro;
any carbocyclyl or heterocyclyl portion of any of R¹, R², R³, R⁵, R⁶, or any ring formed by taking together R¹ and R² or R² and R³ is optionally and independently substituted on a carbon atom with one or more substituents independently selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C};
any heterocyclyl portion of any of R¹, R², R³, R⁵, R⁶, or any ring formed by taking together R¹ and R² or R² and R³ is optionally and independently substituted on a substitutable nitrogen atom with R^{F};
each R^{F} is independently selected from —(C₁-C₆ alkyl), —(C₁-C₆ haloalkyl), —(C₁-C₆ hydroxyalkyl), —(C₀-C₆ alkylene)-carbocyclyl, —(C₀-C₆ alkylene)-heterocyclyl, —S(O)₁₋₂—(C₁-C₆ alkyl), —S(O)₁₋₂—(C₀-C₆ alkylene)-carbocyclyl, —S(O)₁₋₂—(C₀-C₆ alkylene)-heterocyclyl, —C(O)—(C₁-C₆ alkyl), —C(O)—(C₀-C₆ alkylene)-carbocyclyl, —C(O)H, —C(O)—(C₀-C₆ alkylene)-heterocyclyl, —(C₀-C₆ alkylene)-C(O)₂—(C₁-C₆ alkyl), —(C₁-C₆ alkylene)-NR^{B}R^{B′} and —C(O)N(R^{D})(R^{E});
any carbocyclyl or heterocyclyl portion of R^{A}, R^{B}, R^{B′}, R^{C}, R^{D}, R^{E}, R^{F}, any cycloalkyl portion of R^{6′}, or any substituent of R¹, R², R³, R⁵, R⁶ is optionally and independently substituted on a carbon atom with a one or more substituents independently selected from fluoro, chloro, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂; and any heterocyclyl portion of R^{A}, R^{B}, R^{B′}, R^{C}, R^{D}, R^{E}, R^{F}, or any heterocyclyl substituent of Rr, R², R³, R⁵, or R⁶ is optionally substituted on a substitutable nitrogen atom with —C₁-C₄ alkyl, or —S(O)₁₋₂—(C₁-C₄ alkyl). The remaining variables are as described and defined in the first through seventh embodiments, or any aspect thereof, or the eighth embodiment, or first through eighth aspects thereof.

In a tenth aspect of the eighth embodiment, the compound is represented by Formula IIa-1:

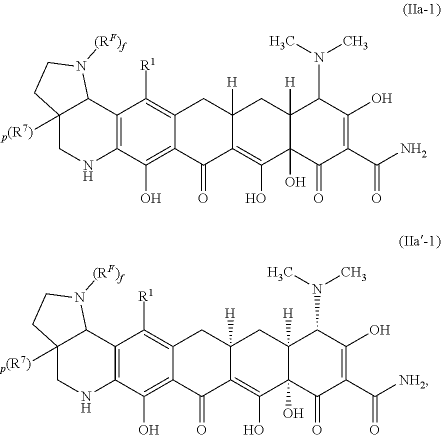

or a pharmaceutically acceptable salt thereof, wherein p is 0 or 1 and R⁷, if present, is —C₁-C₆ alkyl. The remaining variables are as described and defined in the first through seventh embodiments, or any aspect thereof, or the eighth embodiment, or first through ninth aspects thereof.

In an eleventh aspect of the eighth embodiment, the compound is represented by Formula IIb-2:

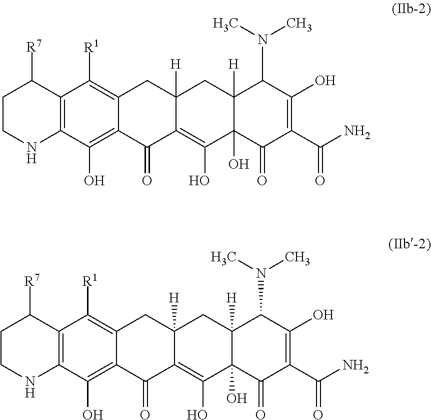

or a pharmaceutically acceptable salt thereof, wherein R⁷ is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}. The remaining variables are as described and defined in the first through seventh embodiments, or any aspect thereof, or the eighth embodiment, or first through tenth aspects thereof.

In a twelfth aspect of the eighth embodiment, any carbocyclyl or heterocyclyl portion of any ring formed by taking together R¹ and R² or R² and R³ is optionally and independently substituted on a carbon atom with one or more substituents independently selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl) and —(C₀-C₆ alkylene)-NR^{B}R^{B′}. The remaining variables are as described and defined in the first through seventh embodiments, or any aspect thereof, or the eighth embodiment, or first through eleventh aspects thereof.

In a ninth embodiment of the invention, the compound administered in the method of treating a hematological cancer is a compound represented by Formula IIc:

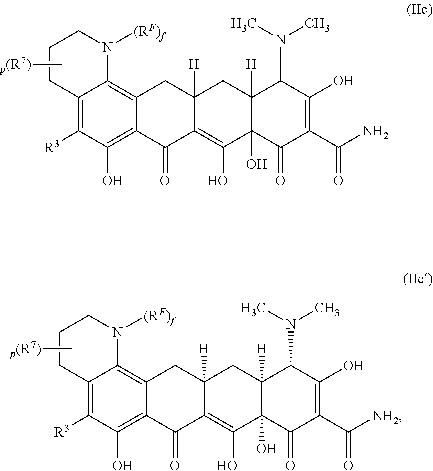

or a pharmaceutically acceptable salt thereof, wherein R⁷, if present, is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}; p is 0 or 1; and f is 0 or 1. Values and alternative values for the remaining variables are as described and defined in the first through eighth embodiments, or any aspect thereof.

In a first aspect of the ninth embodiment, p is 1. The remaining variables are as described and defined in the first through eighth embodiments, or any aspect thereof, or the ninth embodiment.

In a second aspect of the ninth embodiment, the compound is represented by Formula IIc-1:

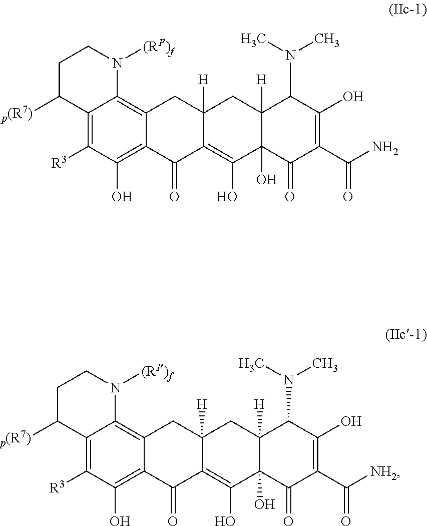

or a pharmaceutically acceptable salt thereof. The variables are as described and defined in the first through eighth embodiments, or any aspect thereof, or the ninth embodiment, or first aspect thereof.

In a third aspect of the ninth embodiment, R⁷, if present, is selected from —(C₀-C₆ alkylene)-(C₃-C₁₀ carbocyclyl), —(C₀-C₆ alkylene)-(4-13 membered heterocyclyl) and —(C₀-C₆ alkylene)-NR^{B}R^{B′}. The remaining variables are as described and defined in the first through eighth embodiments, or any aspect thereof, or the ninth embodiment, or first or second aspect thereof.

In a fourth aspect of the ninth embodiment, R⁷, if present, is —NR^{B}R^{B′}. The remaining variables are as described and defined in the first through eighth embodiments, or any aspect thereof, or the ninth embodiment, or first through third aspects thereof.

In a tenth embodiment of the invention, the compound administered in the method of treating a hematological cancer is a compound of Formula Ia:

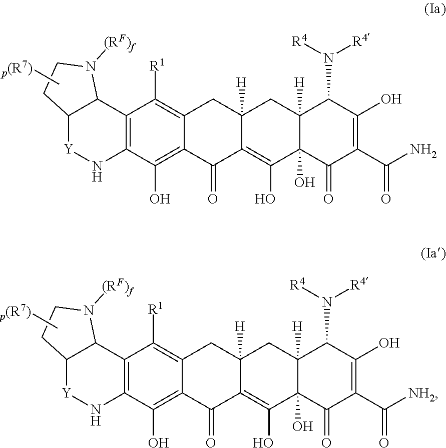

or a pharmaceutically acceptable salt thereof, wherein:

each R⁷, if present, is independently selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C};

p is 0, 1, 2, 3 or 4;

Y is C(O) or C(R⁸)₂ wherein each R⁸ is independently selected from hydrogen, —(C₁-C₆)alkyl and —(C₃-C₆ cycloalkyl); and

f is 0 or 1. Values and alternative values for the variables are as described and defined in the first through ninth embodiments, or any aspect thereof.

In a first aspect of the tenth embodiment, p is 0. The remaining variables are as described and defined in the first through ninth embodiments, or any aspect thereof, or the tenth embodiment.

In a second aspect of the tenth embodiment, each R⁸ is hydrogen. The remaining variables are as described and defined in the first through ninth embodiments, or any aspect thereof, or the tenth embodiment, or first aspect thereof.

In an eleventh embodiment of the invention, the compound administered in the method of treating a hematological cancer is a compound of Formula I, or a pharmaceutically acceptable salt thereof, wherein X is C(R²); and R² is optionally substituted —(C₀-C₁ alkylene)-(4-6-membered heterocyclyl). Values and alternative values for the variables are as described and defined in the first through tenth embodiments, or any aspect thereof.

In a first aspect of the eleventh embodiment, R³ is hydrogen. The remaining variables are as described and defined in the first through tenth embodiments, or any aspect thereof, or the eleventh embodiment.

In a second aspect of the eleventh embodiment, R² is optionally substituted —(C₀-C₁ alkylene)-pyrrolidinyl. The remaining variables are as described and defined in the first through tenth embodiments, or any aspect thereof, or the eleventh embodiment, or first aspect thereof.

In a third aspect of the eleventh embodiment, R² is optionally substituted pyrrolidin-2-yl. The remaining variables are as described and defined in the first through tenth embodiments, or any aspect thereof, or the eleventh embodiment, or first or second aspect thereof.

In a fourth aspect of the eleventh embodiment, R² is optionally substituted —(C₁ alkylene)-(pyrrolidin-1-yl). The remaining variables are as described and defined in the first through tenth embodiments, or any aspect thereof, or the eleventh embodiment, or first through third aspects thereof.

In a twelfth embodiment of the invention, the compound administered in the method of treating a hematological is a compound of Formula Ib:

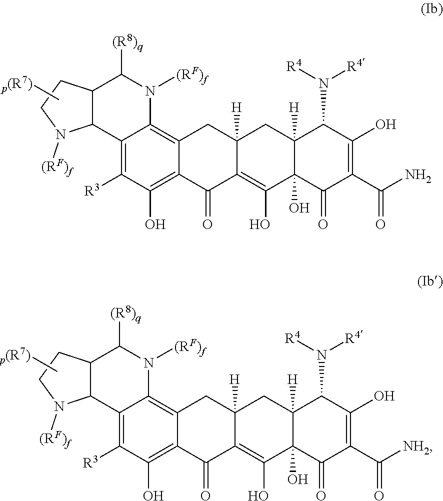

or a pharmaceutically acceptable salt thereof, wherein:

each R⁷ and R⁸, if present, is independently selected from halo, ═O, C₁-C₄ fluoroalkyl, C₁-C₄ alkyl, C₃-C₁₀ carbocyclyl, a 4-13 membered heterocyclyl, OR^{A}, —(C₀-C₆ alkylene)-NR^{B}R^{B′}, and S(O)₀₋₂R^{C};

p is 0, 1, 2, 3 or 4;

q is 0, 1 or 2; and

each f is independently 0 or 1. Values and alternative values for the variables are as described and defined in the first through eleventh embodiments, or any aspect thereof.

In a first aspect of the twelfth embodiment, p and q are each 0. The remaining variables are as described and defined in the first through eleventh embodiments, or any aspect thereof, or the twelfth embodiment.

In a second aspect of the twelfth embodiment, R³ is hydrogen. The remaining variables are as described and defined in the first through eleventh embodiments, or any aspect thereof, or the twelfth embodiment, or first aspect thereof.

In a thirteenth embodiment of the invention, the compound administered in the method of treating a hematological cancer is a compound represented by Formula Ic:

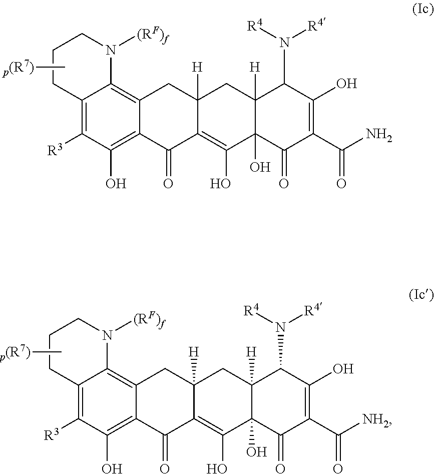

or a pharmaceutically acceptable salt thereof, wherein R⁷, if present, is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}; p is 0 or 1; and f is 0 or 1. Values and alternative values for the remaining variables are as described and defined in the first through twelfth embodiments, or any aspect thereof.

In a first aspect of the thirteenth embodiment, p is 1. The remaining variables are as described and defined in the first through twelfth embodiments, or any aspect thereof, or the thirteenth embodiment.

In a second aspect of the thirteenth embodiment, the compound is represented by Formula Ic-1:

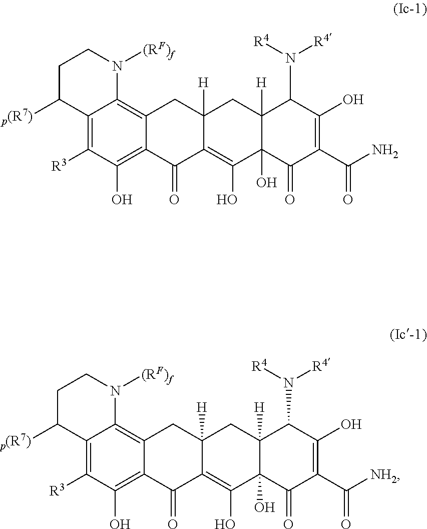

or a pharmaceutically acceptable salt thereof. The variables are as described and defined in the first through twelfth embodiments, or any aspect thereof, or the thirteenth embodiment, or first aspect thereof.

In a third aspect of the thirteenth embodiment, R⁷, if present, is selected from —(C₀-C₆ alkylene)-(C₃-C₁₀ carbocyclyl), —(C₀-C₆ alkylene)-(4-13 membered heterocyclyl) and —(C₀-C₆ alkylene)-NR^{B}R^{B′}. The remaining variables are as described and defined in the first through twelfth embodiments, or any aspect thereof, or the thirteenth embodiment, or first or second aspect thereof.

In a fourth aspect of the thirteenth embodiment, R⁷, if present, is —NR^{B}R^{B′}. The remaining variables are as described and defined in the first through twelfth embodiments, or any aspect thereof, or the thirteenth embodiment, or first through third aspects thereof.

In a fourteenth embodiment of the invention, the compound administered in the method of treating a hematological cancer is a compound represented by Formula I, or a pharmaceutically acceptable salt thereof, wherein X is N and R³ is hydrogen. Values and alternative values for the remaining variables are as described and defined in the first through thirteenth embodiments, or any aspect thereof.

In a first aspect of the fourteenth embodiment, R¹ is selected from hydrogen and NR^{B}R^{B′}. The remaining variables are as described and defined in the first through thirteenth embodiments, or any aspect thereof, or the fourteenth embodiment.

In a fifteenth embodiment of the invention, the compound administered in the method of treating a hematological cancer is a compound of Formula I, or a pharmaceutically acceptable salt thereof, wherein X is C(R²) and R² is (C₁ alkylene)-NR^{B}R^{B′}. Values and alternative values for the remaining variables are as described and defined in the first through fourteenth embodiments, or any aspect thereof.

In a first aspect of the fifteenth embodiment, R^{B} and R^{B′} are each independently selected from hydrogen and —(C₁-C₆ alkyl). The remaining variables are as described and defined in the first through fourteenth embodiments, or any aspect thereof, or the fifteenth embodiment.

In a sixteenth embodiment of the invention, the compound administered in the method of treating a hematological cancer is a compound represented by Formula Id:

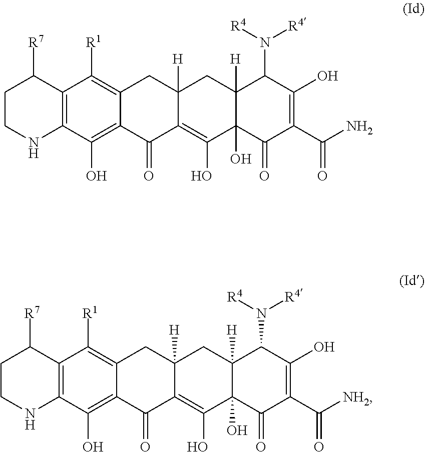

or a pharmaceutically acceptable salt thereof, wherein R⁷ is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}. Values and alternative values for the variables are as defined in the first through fifteenth embodiments, or any aspect thereof.

In a first aspect of the sixteenth embodiment, R⁷ is 4-6 membered heterocyclyl or —NR^{B}R^{B′}. The remaining variables are as described and defined in the first through fifteenth embodiments, or any aspect thereof, or the sixteenth embodiment.

In a seventeenth embodiment of the invention, the compound administered in the method of treating a hematological cancer is a compound represented by Formula Ie:

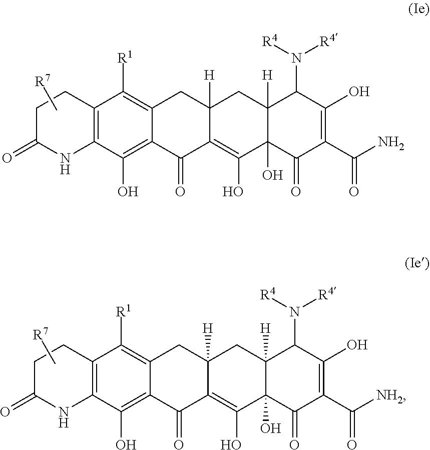

or a pharmaceutically acceptable salt thereof, wherein R⁷ is selected from halo, ═O, C₁-C₄ fluoroalkyl, C₁-C₄ alkyl, —(C₀-C₆ alkylene)-(C₃-C₁₀ carbocyclyl), —(C₀-C₆ alkylene)-(4-13 membered heterocyclyl), OR^{A}, —(C₀-C₆ alkylene)-NR^{B}R^{B′}, and S(O)₀₋₂R^{C}. Values and alternative values for the variables are as defined in the first through sixteenth embodiments, or any aspect thereof.

In a first aspect of the seventeenth embodiment, R⁷ is 4-6 membered heterocyclyl or —NR^{B}R^{B′}. The remaining variables are as described and defined in the first through sixteenth embodiments, or any aspect thereof, or the seventeenth embodiment.

In an additional aspect of any of the preceding embodiments, or any aspect thereof, each R^{A} is independently selected from hydrogen, —(C₁-C₆ alkyl), —(C₀-C₆ alkylene)-carbocyclyl, —(C₀-C₆ alkylene)-heterocyclyl, —S—(C₁-C₆ alkyl), —S—(C₀-C₆ alkylene)-carbocyclyl, —S—(C₀-C₆ alkylene)-heterocyclyl, —C(O)—(C₁-C₆ alkyl), —C(O)—(C₀-C₆ alkylene)-carbocyclyl, —C(O)—(C₀-C₆ alkylene)-heterocyclyl, and —C(O)N(R^{D})(R^{E}). The chemical moiety indicated when f in —N(R^{F})_{f}— is 0 in the structural formulae described herein is —N(H)—. Similarly, when q in —(R⁸)_{q} is 0, it means that the carbon atom attached to —(R⁸)_{q} is attached to two hydrogen atoms.

An eighteenth embodiment of the invention is a compound of Formula (III):

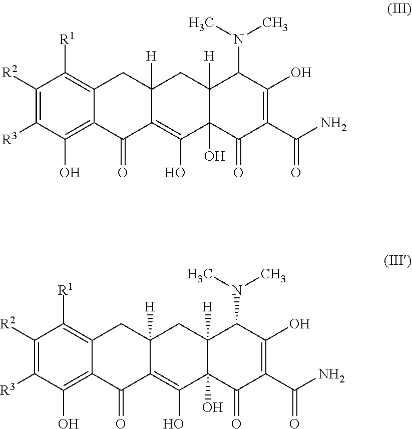

or a pharmaceutically acceptable salt thereof, wherein:

R¹ is selected from hydrogen, bromo, fluoro, chloro, C₁-C₆ alkyl, —O—C₁-C₆ alkyl, —S(O)ₘ—C₁-C₆ alkyl, C₃-C₇ cycloalkyl, —O—C₃-C₇ cycloalkyl, —S(O)ₘ—C₃-C₇ cycloalkyl, —CN, —NR^{G}R^{G′}, and —NH—C(O)—(C₁-C₆ alkylene)-NR^{G}R^{G′}, wherein each alkyl, alkylene or cycloalkyl in the group represented by R¹ is optionally substituted with fluoro;

R² is selected from fluoro, —C₁-C₆ alkyl, and —[C(R^{H})(R^{H})]ₘ—NR^{I}R^{I′};

R³ is selected from hydrogen, fluoro, bromo, —CN, —[C(R^{H})(R^{H})]ₙ—NR^{I}R^{I′}, —NR^{G}R^{G′}, NO₂, —NH—C(O)—C₁-C₄ alkylene-NR^{G}R^{G′}, C₁-C₆ alkyl, —NH—C(O)—C₁-C₆alkyl, —NH—S(O)ₘ—C₁-C₆ alkyl, —NH—S(O)ₘ—C₃-C₁₀ carbocyclyl, —NH—S(O)ₘ-(4-13 membered) heterocyclyl;

each R^{G} and R^{G′} is independently selected from hydrogen and C₁-C₄ alkyl; or

R^{G} and R^{G′} taken together with the nitrogen atom to which they are bound form a (4-7 membered) heterocylic ring optionally comprising one additional heteroatom selected from N, S and O, wherein the (4-7 membered) heterocylic ring is optionally substituted with fluoro, chloro, —OH, fluoro-substituted C₁-C₄ alkyl, —C₁-C₄ alkyl, or —C₁-C₄ alkylene-O—C₁-C₄ alkyl, and is optionally benzofused;

each R^{H} and R^{H′} is independently selected from hydrogen, C₁-C₄ alkyl, and C₃-C₁₀ carbocyclyl;

each R^{I} is selected from hydrogen, C₁-C₁₂ alkyl, —C₀-C₆ alkylene-C₃-C₁₀ carbocyclyl, and —C₀-C₆ alkylene-(4-13 membered) heterocyclyl;

each R^{I} is selected from hydrogen, C₁-C₈ alkyl, —C₀-C₆ alkylene-C₃-C₁₀ carbocyclyl, —C₀-C₆ alkylene-(4-13 membered) heterocyclyl, —C(O)—C₁-C₆ alkyl, —C₀-C₆ alkylene-C(O)—NR^{G}R^{G′}, —C(O)—C₁-C₆ alkylene-NR^{G}R^{G′}, —C₂-C₆ alkylene-NR^{G}R^{G′}, —S(O)ₘ—C₁-C₆ alkyl, —S(O)ₘ—C₃-C₁₀ carbocyclyl, and —S(O)ₘ-(4-13 membered) heterocyclyl, wherein each alkyl, carbocyclyl, alkylene or heterocyclyl in the group represented by R^{I} or R^{I′} is optionally and independently substituted with one or more substituents independently selected from fluoro, chloro, —OH, —O—C₁-C₄ alkyl, C₁-C₄ alkyl, fluoro-substituted-C₁-C₄ alkyl, —NR^{G}R^{G′}, C₃-C₁₀ carbocyclyl and a (4-13 membered) heterocyclyl; or

R^{I} and R^{I′} taken together with the nitrogen atom to which they are bound form a (4-7 membered) monocyclic heterocylic ring, or a (6-13 membered) bicyclic, spirocyclic or bridged heterocylic ring, wherein the (4-7 membered) monocyclic heterocylic ring, or the (6-13 membered) bicyclic, spirocyclic or bridged heterocyclic ring optionally comprises 1 to 4 additional heteroatoms independently selected from N, S and O; and wherein the (4-7 membered) monocyclic heterocylic ring, or the (6-13 membered) bicyclic, spirocyclic or bridged heterocyclic ring is optionally substituted with one or more substituents independently selected from C₃-C₁₀ carbocyclyl, (4-13 membered) heterocyclyl, fluoro, chloro, —OH, C₁-C₄ fluoroalkyl, C₁-C₄ alkyl, —O—C₃-C₁₀ carbocyclyl, —O-(4-13 membered) heterocyclyl, —C₀-C₄ alkyl-O—C₁-C₄ alkyl, —C₀-C₄ alkyl-O—C₁-C₄ fluoroalkyl, ═O, —C(O)—C₁-C₄ alkyl, —C(O) NR^{G}R^{G′}, —N(R^{G})—C(O)—C₁-C₄ alkyl, and —C₀-C₄ alkylene-NR^{G}R^{G′}, and wherein each carbocyclyl or heterocyclyl substituent is optionally substituted with fluoro, chloro, —OH, C₁-C₄ fluoroalkyl, C₁-C₄ alkyl, —O—C₁-C₄ alkyl, —O—C₁-C₄ fluoroalkyl, —NH₂, —NH(C₁-C₄ alkyl), or —N(C₁-C₄ alkyl)₂;

m is 0, 1 or 2; and

n is 1 or 2,

In a first aspect of the eighteenth embodiment, R¹ is hydrogen, bromo, fluoro, chloro, C₁-C₆ alkyl, —O—C₁-C₆ alkyl, —S(O)ₘ—C₁-C₆ alkyl, C₃-C₇ cycloalkyl, —O—C₃-C₇ cycloalkyl, —S(O)ₘ—C₃-C₇ cycloalkyl, —CN, —NR^{G}R^{G′} or —NH—C(O)—(C₁-C₆ alkylene)-NR^{G}R^{G′}.

In some embodiments, each alkyl, alkylene or cycloalkyl in the group represented by R¹ is optionally substituted with fluoro. In other embodiments, R¹ is fluoro, chloro, —CN or —N(CH₃)₂. In other embodiments, R¹ is fluoro, chloro or —N(CH₃)₂. In other embodiments, R¹ is fluoro. In other embodiments, R¹ is chloro. In other embodiments, R¹ is —N(CH₃)₂. In other embodiments, R¹ is hydrogen. The remaining variables are as described and defined in the eighteenth embodiment.

In a second aspect of the eighteenth embodiment, R² is fluoro, —C₁-C₆ alkyl, or —[C(R^{H})(R^{H′})]ₘ—N(R^{I})(R^{I′}). In other embodiments, R² is fluoro, methyl, —CH(R^{H})—N(R^{I})(R^{I′}), —(CH₂)₂—N(R^{I})(R^{I′}), —NH(pyridyl), —NH(C₁-C₈ alkyl), —NHC(O)—C₁-C₃ alkylene-piperidine, —NHC(O)—C₁-C₃ alkylene-pyrrolidine or —NHS(O)₂-phenyl, wherein each piperidine and each pyrrolidine in the group represented by R² is optionally substituted with one or more —C₁-C₆ alkyl. In other embodiments, R² is fluoro, methyl or —CH(R^{H})—N(R^{I})(R^{I′}). In other embodiments, R² is —CH(R^{H})—N(R^{I})(R^{I′}). In other embodiments, R² is fluoro. In other embodiments, R² is —NHR^{I′}. The remaining variables are as described and defined in the eighteenth embodiment, or the first aspect thereof.

In a third aspect of the eighteenth embodiment, R³ is hydrogen, fluoro, bromo, —CN, —[C(R^{H})(R^{H})]ₙ—N(R^{I})(R^{I′}), —NR^{G}R^{G′}, NO₂, —NH—C(O)—C₁-C₄ alkylene-N(R^{I})(R^{I′}), C₁-C₆ alkyl, —NH—C(O)—C₁-C₆ alkyl, —NH—S(O)ₘ—C₁-C₆ alkyl, —NH—S(O)ₘ—C₃-C₁₀ carbocyclyl or —NH—S(O)ₘ-(4-13 membered) heterocyclyl. In other embodiments, R³ is hydrogen, NH₂ or —CH₂—NH—CH₂—C(CH₃)₃. In other embodiments, R³ is hydrogen. In other embodiments, R³ is —[C(R^{H})(R^{H})]ₙ—N(R^{I})(R^{I′}) or —NR^{G}R^{G′}. The remaining variables are as described and defined in the eighteenth embodiment, or the first or second aspect thereof.

In a fourth aspect of the eighteenth embodiment, each R^{H} and R^{H′} is independently selected from hydrogen, C₁-C₄ alkyl, and C₃-C₁₀ carbocyclyl. In other embodiments, R^{H} is hydrogen or methyl. The remaining variables are as described and defined in the eighteenth embodiment, or the first, second, or third aspect thereof.

In a fifth aspect of the eighteenth embodiment, R^{I} is hydrogen, C₁-C₁₂ alkyl, —C₀-C₆ alkylene-C₃-C₁₀ carbocyclyl, or —C₀-C₆ alkylene-(4-13 membered) heterocyclyl. In some embodiments, each alkyl, carbocyclyl, alkylene or heterocyclyl in the group represented by R^{I} is optionally and independently substituted with one or more substituents independently selected from fluoro, chloro, —OH, —O—C₁-C₄ alkyl, C₁-C₄ alkyl, fluoro-substituted-C₁-C₄ alkyl, —NR^{G}R^{G′}, C₃-C₁₀ carbocyclyl and a (4-13 membered) heterocyclyl. In other embodiments, R^{I} is hydrogen, C₁-C₃ straight chained alkyl, C₁-C₃ straight chained fluoroalkyl, cyclopropyl or —CH₂-cyclopropyl. In other embodiments, R^{I} is hydrogen, C₁-C₃ straight chained alkyl or —CH₂-cyclopropyl. The remaining variables are as described and defined in the eighteenth embodiment, or the first through fourth aspect thereof.

In a sixth aspect of the eighteenth embodiment, R^{I′} is hydrogen, C₁-C₈ alkyl, —C₀-C₆ alkylene-C₃-C₁₀ carbocyclyl, —C₀-C₆ alkylene-(4-13 membered) heterocyclyl, —C(O)—C₁-C₆ alkyl, —C₀-C₆ alkylene-C(O)NR^{G}R^{G′}, —C(O)—C₁-C₆ alkylene-NR^{G}R^{G′}, —C₂-C₆ alkylene-NR^{G}R^{G′}, —S(O)ₘ—C₁-C₆ alkyl, —S(O)ₘ—C₃-C₁₀ carbocyclyl or —S(O)ₘ-(4-13 membered) heterocyclyl. In some embodiments, when R² is hydrogen or C₁-C₂ alkyl, R³ is additionally benzyl. In other embodiments, each alkyl, carbocyclyl, alkylene or heterocyclyl in the group represented by R^{I′} is optionally and independently substituted with one or more substituents independently selected from fluoro, chloro, —OH, —O—C₁-C₄ alkyl, C₁-C₄ alkyl, fluoro-substituted-C₁-C₄ alkyl, —NR^{G}R^{G′}, C₃-C₁₀ carbocyclyl and a (4-13 membered) heterocyclyl. In other embodiments, R^{I′} is hydrogen, C₁-C₈ alkyl, —CH₂—CHF₂, —C₂-C₆ alkylene-O—C₁-C₃ alkyl, —C₃-C₁₀ cycloalkyl, —C₃-C₁₀cycloalkyl-substituted C₁-C₃ alkyl, cyclopropyl-substituted cyclopropyl, —(CH₂)₂-phenyl or —S(O)₂-phenyl. In other embodiments, R^{I′} is hydrogen, C₁-C₈ alkyl, —CH₂—CHF₂, —C₁-C₆ alkylene-O—C₁-C₃ alkyl, C₃-C₁₀ cycloalkyl, C₃-C₁₀ cycloalkyl-substituted C₁-C₃ alkyl, or —(CH₂)₂-phenyl, and when R^{I} is hydrogen or —C₁-C₂ alkyl, R^{I′} is additionally benzyl. In other embodiments, R^{I′} is selected from hydrogen, C₁-C₈ alkyl, —CH₂—CHF₂, —C₁-C₆ alkylene-O—C₁-C₃ alkyl, C₃-C₁₀ cycloalkyl, —(CH₂)₂-phenyl and C₃-C₁₀ cycloalkyl-substituted C₁-C₃ alkyl, wherein each cycloalkyl in the group represented by R^{I′} is optionally substituted with —C₁-C₃ alkyl or optionally benzofused. The remaining variables are as described and defined in the eighteenth embodiment, or the first through fifth aspect thereof.

In a seventh aspect of the eighteenth embodiment, R^{I} and R^{I′} taken together with the nitrogen atom to which they are bound form a (4-7 membered) monocyclic heterocylic ring, or a (6-13 membered) bicyclic, spirocyclic or bridged heterocylic ring, wherein the (4-7 membered) monocyclic heterocylic ring, or the (6-13 membered) bicyclic, spirocyclic or bridged heterocyclic ring optionally comprises 1 to 4 additional heteroatoms independently selected from N, S and O. In some embodiments, the (4-7 membered) monocyclic heterocylic ring, or the (6-13 membered) bicyclic, spirocyclic or bridged heterocyclic ring is optionally substituted with one or more substituents independently selected from C₃-C₁₀ carbocyclyl, (4-13 membered) heterocyclyl, fluoro, chloro, —OH, C₁-C₄ fluoroalkyl, C₁-C₄ alkyl, —O—C₃-C₁₀ carbocyclyl, —O-(4-13 membered) heterocyclyl, —C₀-C₄ alkyl-O—C₁-C₄ alkyl, —C₀-C₄ alkyl-O—C₁-C₄ fluoroalkyl, ═O, —C(O)—C₁-C₄ alkyl, —C(O)N R^{G}R^{G′}, —N(R^{G})—C(O)—C₁-C₄ alkyl, and —C₀-C₄ alkylene-N R^{G}R^{G′}, and wherein each carbocyclyl or heterocyclyl substituent is optionally substituted with fluoro, chloro, —OH, C₁-C₄ fluoroalkyl, C₁-C₄ alkyl, —O—C₁-C₄ alkyl, —O—C₁-C₄ fluoroalkyl, —NH₂, —NH(C₁-C₄ alkyl), or —N(C₁-C₄ alkyl)₂. In other embodiments, R^{I} and R^{I′} taken together with the nitrogen atom to which they are bound form a ring selected from pyrrolidine, piperidine, piperazine and morpholine, wherein the ring is optionally substituted with one or more substituents independently selected from —OH, —C₁-C₃ alkyl and —C₁-C₃ alkylene-O—C₁-C₃ alkyl, and wherein the ring is optionally benzofused or spirofused to cyclopropyl. In other embodiments, R^{I} and R^{I′} taken together with the nitrogen atom to which they are bound form a ring selected from pyrrolidine and piperidine, wherein the ring is optionally substituted with one or more substituents independently selected from fluoro, C₁-C₃ alkyl and —C₁-C₃ alkylene-O—C₁-C₃ alkyl, and wherein the ring is optionally benzofused or spirofused to cyclopropyl. The remaining variables are as described and defined in the eighteenth embodiment, or the first through sixth aspect thereof.

In an eighth aspect of the eighteenth embodiment, R^{G} and R^{G′} are independently hydrogen or C₁-C₄ alkyl. In other embodiments, R^{G} and R^{G′} taken together with the nitrogen atom to which they are bound form a (4-7 membered) heterocylic ring optionally comprising one additional heteroatom selected from N, S and O, wherein the (4-7 membered) heterocylic ring is optionally substituted with fluoro, chloro, —OH, fluoro-substituted C₁-C₄ alkyl, —C₁-C₄ alkyl, or —C₁-C₄ alkylene-O— C₁-C₄ alkyl, and is optionally benzofused. The remaining variables are as described and defined in the eighteenth embodiment, or the first through seventh aspect thereof.

A nineteenth embodiment of the invention is a compound of Structural Formula (III) or (III′), wherein R² is fluoro,

methyl, —CH(R^{H})—N(R^{I})(R^{I′}), —(CH₂)₂—N(R^{I})(R^{I′}), —NH(pyridyl), —NH(C₁-C₈ alkyl), —NHC(O)—C₁-C₃alkylene-piperidine, —NHC(O)—C₁-C₃ alkylene-pyrrolidine or —NHS(O)₂-phenyl, and each piperidine and each pyrrolidine in the group represented by R² is optionally substituted with one or more —C₁-C₆ alkyl; R^{H} is hydrogen or methyl; R^{I} is hydrogen, C₁-C₃ straight chained alkyl, C₁-C₃ straight chained fluoroalkyl, cyclopropyl or —CH₂-cyclopropyl; R^{I′} is hydrogen, C₁-C₈ alkyl, —CH₂—CHF₂, —C₂-C₆ alkylene-O—C₁-C₃ alkyl, —C₃-C₁₀ cycloalkyl, —C₃-C₁₀cycloalkyl-substituted C₁-C₃ alkyl, cyclopropyl-substituted cyclopropyl, —(CH₂)₂-phenyl or —S(O)₂-phenyl, and when R^{I} is hydrogen or C₁-C₂ alkyl, R^{I′} is additionally benzyl; or R^{I} and R^{I′} taken together with the nitrogen atom to which they are bound form a ring selected from pyrrolidine, piperidine, piperazine or morpholine, wherein the ring is optionally substituted with one or more substituents independently selected from —OH, —C₁-C₃ alkyl and —C₁-C₃ alkylene-O—C₁-C₃ alkyl, and wherein the ring is optionally benzofused or spirofused to cyclopropyl. The remaining variables are as described and defined in the eighteenth embodiment, or any aspect thereof.

A twentieth embodiment of the invention is a compound of Structural Formula (III) or (III′), wherein R² is fluoro, methyl or —CH(R^{H})—N(R^{I})(R^{I′}); R^{H} is hydrogen or methyl; R^{I} is hydrogen, C₁-C₃ straight chained alkyl or —CH₂-cyclopropyl; R^{I′} is hydrogen, C₁-C₈ alkyl, —CH₂—CHF₂, —C₁-C₆ alkylene-O—C₁-C₃ alkyl, C₃-C₁₀ cycloalkyl, or C₃-C₁₀ cycloalkyl-substituted C₁-C₃ alkyl, wherein each cycloalkyl in the group represented by R^{I′} is optionally substituted with —C₁-C₃ alkyl or optionally benzofused, or —(CH₂)₂-phenyl; and when R^{I} is hydrogen or —C₁-C₂ alkyl, R^{I′} is additionally benzyl; or R^{I} and R^{I′} taken together with the nitrogen atom to which they are bound form a ring selected from pyrrolidine and piperidine, wherein the ring is optionally substituted with one or more substituents independently selected from fluoro, —C₁-C₃ alkyl and —C₁-C₃ alkylene-O—C₁-C₃ alkyl, and wherein the ring is optionally benzofused or spirofused to cyclopropyl. The remaining variables are as described and defined in the eighteenth or nineteeth embodiment, or any aspect thereof.

A twenty-first embodiment of the invention is a compound of Structural Formula (III) or (III′), wherein X is fluoro, chloro, —CN or —N(CH₃)₂; and Z is hydrogen, NH₂ or —CH₂—NH—CH₂—C(CH₃)₃. The remaining variables are as described and defined in the eighteenth through twentieth embodiments, or any aspect thereof.

A twenty-second embodiment of the invention is a compound of Structural Formula (III) or III′), wherein

R¹ is selected from —OCH₃, —CF₃, Cl, F, and —N(CH₃)₂;

Z is hydrogen and when R¹ is F, Z is additionally selected from hydrogen, —NH₂, —NH(C₁-C₂ alkyl), and —N(C₁-C₂ alkyl)₂; and

R² is —CH₂—NR^{I}R^{I′};

wherein

R^{I} is selected from hydrogen and C₁-C₃ alkyl; and

R^{I′} is selected from hydrogen, C₁-C₈ alkyl, C₀-C₆ alkylene C₃-C₁₀ carbocyclyl, C₀-C₆ alkylene-(4-13 membered) heterocyclyl, and C₂-C₆ alkylene —N(R^{G})(R^{G′}), wherein each carbocyclyl or heterocyclyl in the group represented by R^{I′} is optionally and independently substituted with one or more substituents independently selected from fluoro, —OH, —O—C₁-C₃ alkyl, C₁-C₃ alkyl, fluoro-substituted C₁-C₃ alkyl, —N(R^{G})(R^{G′}), C₃-C₁₀ carbocyclyl or a (4-13 membered) heterocyclyl; or

R^{I} and R^{I′} taken together with the nitrogen atom to which they are bound form a (4-7 membered) saturated monocyclic heterocylic ring, or a (6-13 membered) saturated bicyclic, spirocyclic or bridged heterocylic ring, wherein the (4-7 membered) monocyclic heterocylic ring, or the (6-13 membered) bicyclic, spirocyclic or bridged heterocyclic ring, is optionally substituted with one or more substituents independently selected from C₃-C₁₀ carbocyclyl, (4-13 membered) heterocyclyl, fluoro, —OH, —C₁-C₃ fluoroalkyl, —C₁-C₃ alkyl, —O—C₃-C₁₀ carbocyclyl, —O-(4-13 membered) heterocyclyl, C₀-C₂ alkylene-O—C₁-C₃ alkyl, C₀-C₂ alkylene-O—C₁-C₃ fluoroalkyl, ═O, and C₀-C₄ alkylene-N(R^{G})(R^{G′})), and wherein each carbocyclyl or heterocyclyl substituent is optionally substituted with fluoro, —OH, C₁-C₃ fluoroalkyl, C₁-C₃ alkyl, —O—C₁-C₃ alkyl, —O—C₁-C₃ fluoroalkyl, —NH₂—NH(C₁-C₄ alkyl), or —N(C₁-C₄ alkyl)₂; and

each R^{G} and R^{G′} is independently selected from hydrogen and C₁-C₄ alkyl. The remaining variables are as described and defined in the eighteenth through twenty-first embodiments, or any aspect thereof.

A twenty-third embodiment of the invention is a compound of Structural Formula (III) or (III′), wherein R¹ is —OCH₃. In other embodiments, R¹ is —CF₃. In other embodiments, R¹ is —Cl. In other embodiments, R¹ is —F and R³ is hydrogen. In other embodiments, R¹ is —F and R³ is selected from —NH₂, —NH(C₁-C₂ alkyl), and —N(C₁-C₂ alkyl)₂. In other embodiments, R¹ is —N(CH₃)₂. In other embodiments, R² is —NH^{I′}; and R^{I′} is pyridyl, C₁-C₈ alkyl, —C(O)—C₁-C₃ alkylene-piperidine or —C(O)—C₁-C₃ alkylene-pyrrolidine. Each piperidine or pyrrolidine in the group represented by R^{I′} is optionally substituted with one or more C₁-C₃ alkyl. The remaining variables are as described and defined in the eighteenth through twenty-second embodiments, or any aspect thereof.

A twenty-fourth embodiment of the invention is a compound of Structural Formulae (IV), (IV′), (V), (V′), (Va), (Va′), (VI), (VI′), (VII) or (VII′):

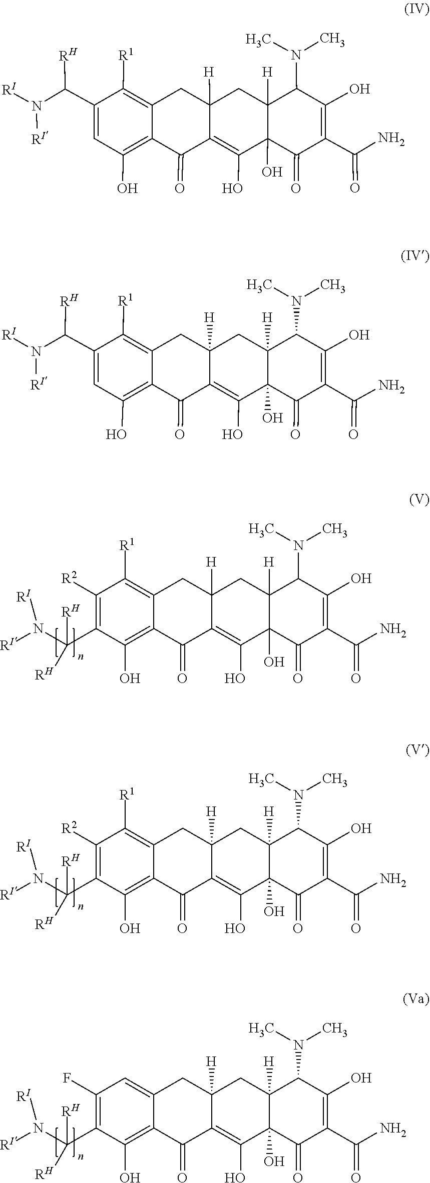

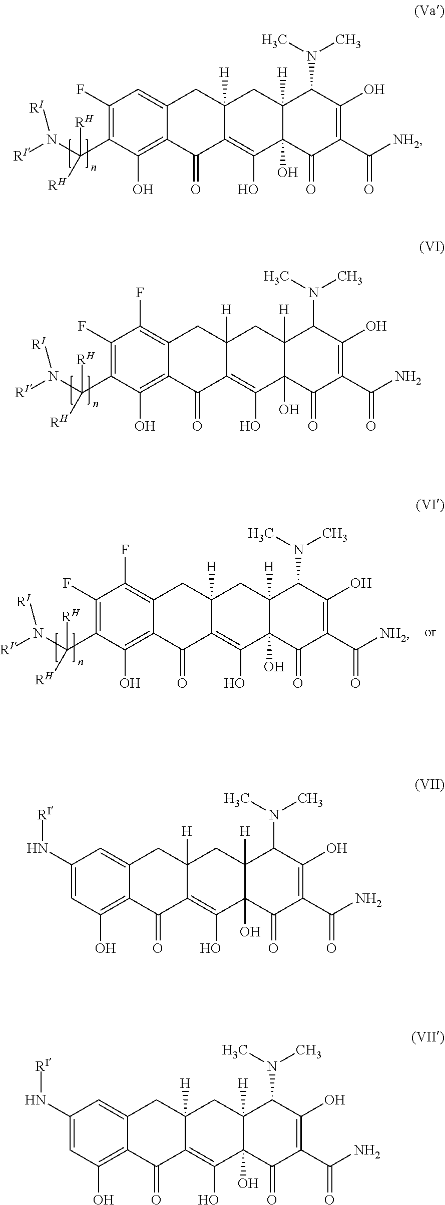

or a pharmaceutically acceptable salt thereof, wherein values and alternative values for the variables are found in the eighteenth to twenty-third embodiments of the invention.

A twenty-fifth embodiment of the invention is a compound of Structural Formulae (IV) or (IV′)

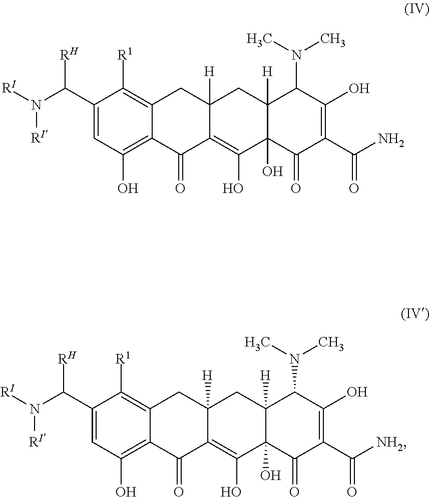

or a pharmaceutically acceptable salt thereof, wherein:

R¹ is selected from, bromo, fluoro, chloro, C₁-C₆ fluoroalkyl, —O—C₁-C₆ alkyl, —S(O)ₘ—C₁-C₆ alkyl, C₃-C₇ cycloalkyl, —O—C₃-C₇ cycloalkyl, —S(O)ₘ—C₃-C₇ cycloalkyl, —CN, and —NH—C(O)—(C₁-C₆ alkylene)-NR^{G}R^{G′}, wherein each alkyl, alkylene or cycloalkyl in the group represented by R¹ is optionally substituted with fluoro;

each R^{G} and R^{G′} is independently selected from hydrogen and C₁-C₄ alkyl; or

R^{G} and R^{G′} taken together with the nitrogen atom to which they are bound form a (4-7 membered) heterocylic ring optionally comprising one additional heteroatom selected from N, S and O, wherein the (4-7 membered) heterocylic ring is optionally substituted with fluoro, chloro, —OH, fluoro-substituted C₁-C₄ alkyl, —C₁-C₄ alkyl, or —C₁-C₄ alkylene-O—C₁-C₄ alkyl, and is optionally benzofused;

each R^{H} and R^{H′} is independently selected from hydrogen, C₁-C₄ alkyl, and C₃-C₁₀ carbocyclyl;

each R^{I} is selected from hydrogen, C₁-C₁₂ alkyl, —C₀-C₆ alkylene-C₃-C₁₀ carbocyclyl, and —C₀-C₆ alkylene-(4-13 membered) heterocyclyl;

each R^{I′} is selected from hydrogen, C₁-C₈ alkyl, —C₀-C₆ alkylene-C₃-C₁₀ carbocyclyl, —C₀-C₆ alkylene-(4-13 membered) heterocyclyl, —C(O)—C₁-C₆ alkyl, —C₀-C₆ alkylene-C(O)—NR^{G}R^{G′}, —C(O)—C₁-C₆ alkylene-NR^{G}R^{G′}, —C₂-C₆ alkylene-NR^{G}R^{G′}, —S(O)ₘ—C₁-C₆ alkyl, —S(O)ₘ—C₃-C₁₀ carbocyclyl, and —S(O)ₘ-(4-13 membered) heterocyclyl, wherein each alkyl, carbocyclyl, alkylene or heterocyclyl in the group represented by R^{I} or R^{I′} is optionally and independently substituted with one or more substituents independently selected from fluoro, chloro, —OH, —O—C₁-C₄ alkyl, C₁-C₄ alkyl, fluoro-substituted-C₁-C₄ alkyl, —NR^{G}R^{G′}, C₃-C₁₀ carbocyclyl and a (4-13 membered) heterocyclyl; or

R^{I} and R^{I′} taken together with the nitrogen atom to which they are bound form a (4-7 membered) monocyclic heterocylic ring, or a (6-13 membered) bicyclic, spirocyclic or bridged heterocylic ring, wherein the (4-7 membered) monocyclic heterocylic ring, or the (6-13 membered) bicyclic, spirocyclic or bridged heterocyclic ring optionally comprises 1 to 4 additional heteroatoms independently selected from N, S and O; and wherein the (4-7 membered) monocyclic heterocylic ring, or the (6-13 membered) bicyclic, spirocyclic or bridged heterocyclic ring is optionally substituted with one or more substituents independently selected from C₃-C₁₀ carbocyclyl, (4-13 membered) heterocyclyl, fluoro, chloro, —OH, C₁-C₄ fluoroalkyl, C₁-C₄ alkyl, —O—C₃-C₁₀ carbocyclyl, —O-(4-13 membered) heterocyclyl, —C₀-C₄ alkyl-O—C₁-C₄ alkyl, —C₀-C₄ alkyl-O—C₁-C₄ fluoroalkyl, ═O, —C(O)—C₁-C₄ alkyl, —C(O) NR^{G}R^{G′}, —N(R^{G})—C(O)—C₁-C₄ alkyl, and —C₀-C₄ alkylene-NR^{G}R^{G′}, and wherein each carbocyclyl or heterocyclyl substituent is optionally substituted with fluoro, chloro, —OH, C₁-C₄ fluoroalkyl, C₁-C₄ alkyl, —O—C₁-C₄ alkyl, —O—C₁-C₄ fluoroalkyl, —NH₂, —NH(C₁-C₄ alkyl), or —N(C₁-C₄ alkyl)₂; and

m is 0, 1 or 2.

In a first aspect of the twenty-fifth embodiment,
R^{H} is selected from hydrogen and methyl;
R^{I} is selected from hydrogen, C₁-C₃ straight chained alkyl, C₁-C₃ straight chained fluoroalkyl, cyclopropyl, and —CH₂-cyclopropyl;
R^{I′} is selected from hydrogen, C₁-C₈ alkyl, —CH₂—CHF₂, —C₂-C₆ alkylene-O—C₁-C₃ alkyl, —C₃-C₁₀ cycloalkyl, —C₃-C₁₀ cycloalkyl-substituted C₁-C₃ alkyl, cyclopropyl-substituted cyclopropyl, —(CH₂)₂-phenyl, and —S(O)₂-phenyl, when R² is hydrogen or C₁-C₂ alkyl, R³ is additionally selected from benzyl; or
R^{I} and R^{I′} taken together with the nitrogen atom to which they are bound form a ring selected from pyrrolidine, piperidine, piperazine or morpholine, wherein the ring is optionally substituted with one or more substituents independently selected from —OH, —C₁-C₃ alkyl and —C₁-C₃ alkylene-O—C₁-C₃ alkyl, and wherein the ring is optionally fused to phenyl or spirofused to cyclopropyl.
In a second aspect of the twenty-fifth embodiment,
R^{H} is selected from hydrogen and methyl;
R^{I} is selected from hydrogen, C₁-C₃ straight chained alkyl and —CH₂-cyclopropyl;
R^{I′} is selected from hydrogen, C₁-C₈ alkyl, —CH₂—CHF₂, —C₁-C₆ alkylene-O—C₁-C₃ alkyl, C₃-C₁₀ cycloalkyl, —(CH₂)₂-phenyl and C₃-C₁₀ cycloalkyl-substituted C₁-C₃ alkyl, wherein each cycloalkyl in the group represented by R³ is optionally substituted with —C₁-C₃ alkyl or optionally benzofused and when R² is hydrogen or —C₁-C₂ alkyl, R³ is additionally selected from benzyl; or
R^{I} and R^{I′} taken together with the nitrogen atom to which they are bound form a ring selected from pyrrolidine and piperidine, wherein the ring is optionally substituted with one or more substituents independently selected from fluoro —C₁-C₃ alkyl and —C₁-C₃ alkylene-O—C₁-C₃ alkyl, and wherein the ring is optionally fused to phenyl or spirofused to cyclopropyl.
In a third aspect of the twenty-fifth embodiment, R¹ is fluoro or chloro.
In a fourth aspect of the twenty-fifth embodiment, the compound used in the method of treating hematological malignancies is selected from any one of the following:

R¹ is fluoro and —CH(R^{H})—NR^{I}R^{I′} is

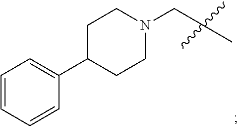 [SMILES: *CN1CCC(c2ccccc2)CC1]

R¹ is fluoro and —CH(R^{H})—NR^{I}R^{I′} is

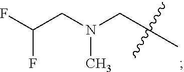 [SMILES: *CN(C)CC(F)F]

R¹ is fluoro and —CH(R^{H})—NR^{I}R^{I′} is

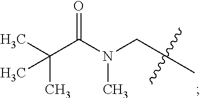 [SMILES: *CN(C)C(=O)C(C)(C)C]

R¹ is fluoro and —CH(R^{H})—NR^{I}R^{I′} is

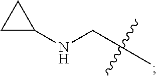 [SMILES: *CNC1CC1]

R¹ is fluoro and —CH(R^{H})—NR^{I}R^{I′} is

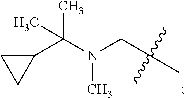 [SMILES: *CN(C)C(C)(C)C1CC1]

R¹ is fluoro and —CH(R^{H})—NR^{I}R^{I′} is

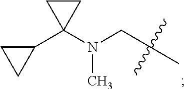 [SMILES: *CN(C)C1(C2CC2)CC1]

R^{I} is fluoro and —CH(R^{H})—NR^{I}R^{I′} is

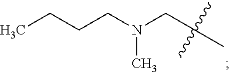 [SMILES: *CN(C)CCCC]

R^{I} is fluoro and —CH(R^{H})—NR^{I}R^{I′} is

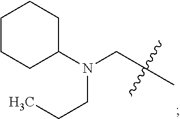 [SMILES: *CN(CCC)C1CCCCC1]

R¹ is fluoro and —CH(R^{H})—NR^{I}R^{I′} is

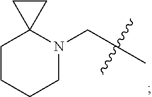 [SMILES: *CN1CCCCC12CC2]

R¹ is fluoro and —CH(R^{H})—NR^{I}R^{I′} is

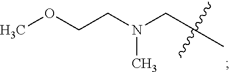 [SMILES: *CN(C)CCOC]

R¹ is fluoro and —CH(R^{H})—NR^{I}R^{I′} is

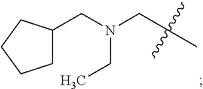 [SMILES: *CN(CC)CC1CCCC1]

R¹ is fluoro and —CH(R^{H})—NR^{I}R^{I′} is

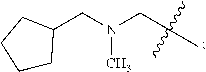 [SMILES: *CN(C)CC1CCCC1]

R¹ is fluoro and —CH(R^{H})—NR^{I}R^{I′} is

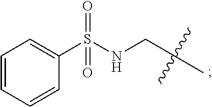 [SMILES: *CNS(=O)(=O)c1ccccc1]

R¹ is fluoro and —CH(R^{H})—NR^{I}R^{I′} is

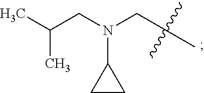 [SMILES: *CN(CC(C)C)C1CC1]

R¹ is fluoro and —CH(R^{H})—NR^{I}R^{I′} is

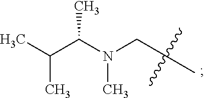 [SMILES: *CN(C)[C@@H](C)C(C)C]

R¹ is fluoro and —CH(R^{H})—NR^{I}R^{I′} is

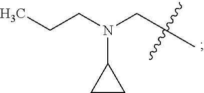 [SMILES: *CN(CCC)C1CC1]

R¹ is fluoro and —CH(R^{H})—NR^{I}R^{I′} is

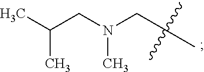 [SMILES: *CN(C)CC(C)C]

R¹ is fluoro and —CH(R^{H})—NR^{I}R^{I′} is

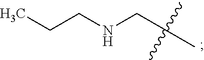 [SMILES: *CNCCC]

R¹ is fluoro and —CH(R^{H})—NR^{I}R^{I′} is

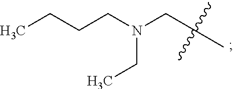 [SMILES: *CN(CC)CCCC]

R¹ is fluoro and —CH(R^{H})—NR^{I}R^{I′} is

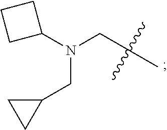 [SMILES: *CN(CC1CC1)C1CCC1]

R¹ is fluoro and —CH(R^{H})—NR^{I}R^{I′} is

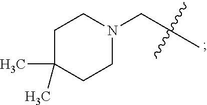 [SMILES: *CN1CCC(C)(C)CC1]

R¹ is fluoro and —CH(R^{H})—NR^{I}R^{I′} is

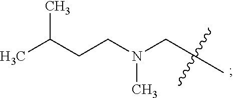 [SMILES: *CN(C)CCC(C)C]

R¹ is fluoro and —CH(R^{H})—NR^{I}R^{I′} is

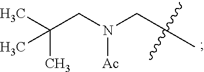 [SMILES: *CN(CC(C)(C)C)C(C)=O]

R¹ is fluoro and —CH(R^{H})—NR^{I}R^{I′} is

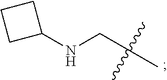 [SMILES: *CNC1CCC1]

R¹ is fluoro and —CH(R^{H})—NR^{I}R^{I′} is

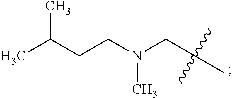 [SMILES: *CN(C)CCC(C)C]

R¹ is fluoro and —CH(R^{H})—NR^{I}R^{I′} is

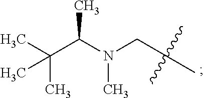 [SMILES: *CN(C)[C@H](C)C(C)(C)C]

R¹ is fluoro and —CH(R^{H})—NR^{I}R^{I′} is

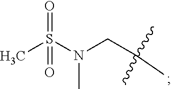 [SMILES: *CN(C)S(C)(=O)=O]

R¹ is fluoro and —CH(R^{H})—NR^{I}R^{I′} is

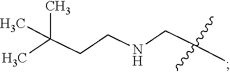 [SMILES: *CNCCC(C)(C)C]

R¹ is fluoro and —CH(R^{H})—NR^{I}R^{I′} is

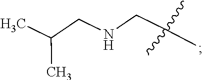 [SMILES: *CNCC(C)C]

R¹ is fluoro and —CH(R^{H})—NR^{I}R^{I′} is

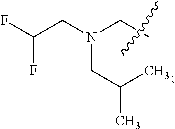

R¹ is fluoro and —CH(R^{H})—NR^{I}R^{I′} is

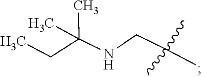 [SMILES: *CNC(C)(C)CC]

R¹ is fluoro and —CH(R^{H})—NR^{I}R^{I′} is

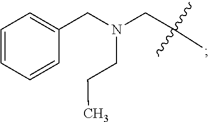 [SMILES: *CN(CCC)Cc1ccccc1]

R¹ is fluoro and —CH(R^{H})—NR^{I}R^{I′} is

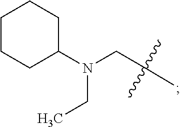 [SMILES: *CN(CC)C1CCCCC1]

R¹ is fluoro and —CH(R^{H})—NR^{I}R^{I′} is

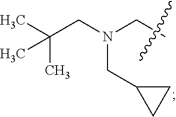

R¹ is fluoro and —CH(R^{H})—NR^{I}R^{I′} is

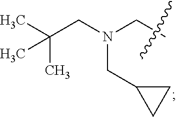

R¹ is fluoro and —CH(R^{H})—NR^{I}R^{I′} is

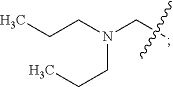

R¹ is fluoro and —CH(R^{H})—NR^{I}R^{I′} is

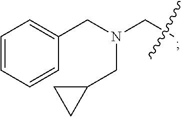

R¹ is fluoro and —CH(R^{H})—NR^{I}R^{I′} is

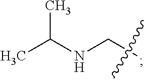

R¹ is fluoro and —CH(R^{H})—NR^{I}R^{I′} is

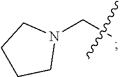

R¹ is fluoro and —CH(R^{H})—NR^{I}R^{I′} is

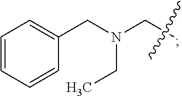

R¹ is fluoro and —CH(R^{H})—NR^{I}R^{I′} is

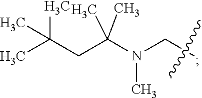

R¹ is fluoro and —CH(R^{H})—NR^{I}R^{I′} is

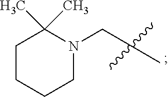 [SMILES: *CN1CCCCC1(C)C]

R¹ is fluoro and —CH(R^{H})—NR^{I}R^{I′} is

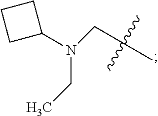 [SMILES: *CN(CC)C1CCC1]

R¹ is fluoro and —CH(R^{H})—NR^{I}R^{I′} is

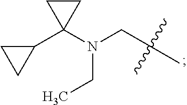 [SMILES: *CN(CC)C1(C2CC2)CC1]

R¹ is fluoro and —CH(R^{H})—NR^{I}R^{I′} is

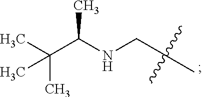 [SMILES: *CN[C@H](C)C(C)(C)C]

R¹ is fluoro and —CH(R^{H})—NR^{I}R^{I′} is

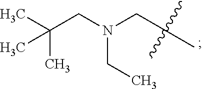 [SMILES: *CN(CC)CC(C)(C)C]

R¹ is fluoro and —CH(R^{H})—NR^{I}R^{I′} is

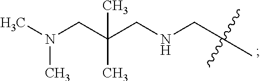 [SMILES: *CNCC(C)(C)CN(C)C]

R¹ is fluoro and —CH(R^{H})—NR^{I}R^{I′} is

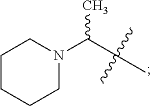 [SMILES: *C(C)N1CCCCC1]

R¹ is fluoro and —CH(R^{H})—NR^{I}R^{I′} is

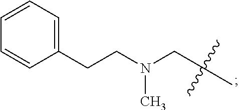 [SMILES: *CN(C)CCc1ccccc1]

R¹ is fluoro and —CH(R^{H})—NR^{I}R^{I′} is

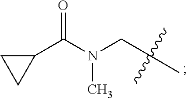 [SMILES: *CN(C)C(=O)C1CC1]

R¹ is fluoro and —CH(R^{H})—NR^{I}R^{I′} is

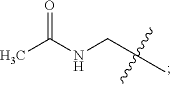 [SMILES: *CNC(C)=O]

R¹ is fluoro and —CH(R^{H})—NR^{I}R^{I′} is

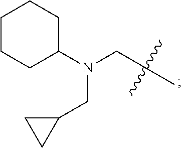 [SMILES: *CN(CC1CC1)C1CCCCC1]

R¹ is fluoro and —CH(R^{H})—NR^{I}R^{I′} is

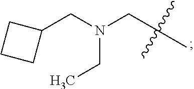 [SMILES: *CN(CC)CC1CCC1]

R¹ is fluoro and —CH(R^{H})—NR^{I}R^{I′} is

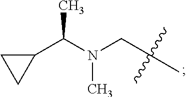 [SMILES: *CN(C)[C@H](C)C1CC1]

R¹ is fluoro and —CH(R^{H})—NR^{I}R^{I′} is

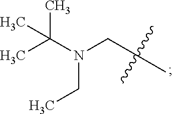 [SMILES: *CN(CC)C(C)(C)C]

R¹ is fluoro and —CH(R^{H})—NR^{I}R^{I′} is

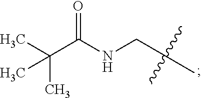 [SMILES: *CNC(=O)C(C)(C)C]

R¹ is fluoro and —CH(R^{H})—NR^{I}R^{I′} is

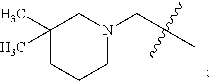 [SMILES: *CN1CCCC(C)(C)C1]

R¹ is fluoro and —CH(R^{H})—NR^{I}R^{I′} is

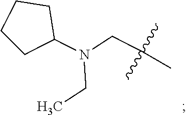 [SMILES: *CN(CC)C1CCCC1]

R¹ is fluoro and —CH(R^{H})—NR^{I}R^{I′} is

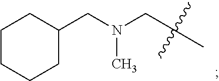 [SMILES: *CN(C)CC1CCCCC1]

R¹ is fluoro and —CH(R^{H})—NR^{I}R^{I′} is

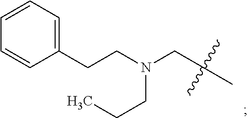 [SMILES: *CN(CCC)CCc1ccccc1]

R¹ is fluoro and —CH(R^{H})—NR^{I}R^{I′} is

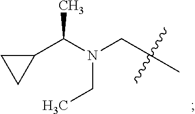 [SMILES: *CN(CC)[C@H](C)C1CC1]

R¹ is fluoro and —CH(R^{H})—NR^{I}R^{I′} is

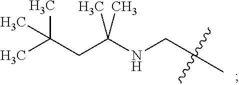 [SMILES: *CNC(C)(C)CC(C)(C)C]

R¹ is fluoro and —CH(R^{H})—NR^{I}R^{I′} is

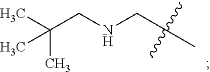 [SMILES: *CNCC(C)(C)C]

R¹ is fluoro and —CH(R^{H})—NR^{I}R^{I′} is

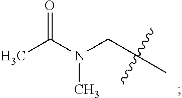 [SMILES: *CN(C)C(C)=O]

R¹ is fluoro and —CH(R^{H})—NR^{I}R^{I′} is

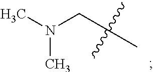 [SMILES: *CN(C)C]

R¹ is fluoro and —CH(R^{H})—NR^{I}R^{I′} is

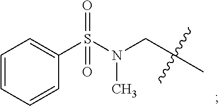 [SMILES: *CN(C)S(=O)(=O)c1ccccc1]

R¹ is fluoro and —CH(R^{H})—NR^{I}R^{I′} is

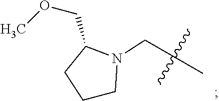 [SMILES: *CN1CCC[C@@H]1COC]

R¹ is fluoro and —CH(R^{H})—NR^{I}R^{I′} is

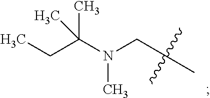 [SMILES: *CN(C)C(C)(C)CC]

R¹ is fluoro and —CH(R^{H})—NR^{I}R^{I′} is

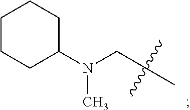 [SMILES: *CN(C)C1CCCCC1]

R¹ is fluoro and —CH(R^{H})—NR^{I}R^{I′} is

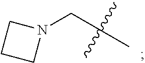 [SMILES: *CN1CCC1]

R¹ is fluoro and —CH(R^{H})—NR^{I}R^{I′} is

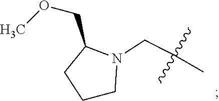 [SMILES: *CN1CCC[C@H]1COC]

R¹ is fluoro and —CH(R^{H})—NR^{I}R^{I′} is

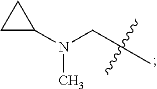 [SMILES: *CN(C)C1CC1]

R¹ is fluoro and —CH(R^{H})—NR^{I}R^{I′} is

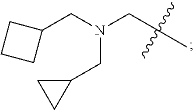 [SMILES: *CN(CC1CCC1)CC1CC1]

R¹ is fluoro and —CH(R^{H})—NR^{I}R^{I′} is

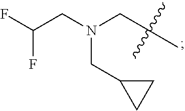 [SMILES: *CN(CC(F)F)CC1CC1]

R¹ is fluoro and —CH(R^{H})—NR^{I}R^{I′} is

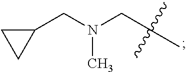 [SMILES: *CN(C)CC1CC1]

R¹ is fluoro and —CH(R^{H})—NR^{I}R^{I′} is

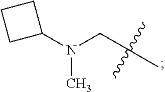 [SMILES: *CN(C)C1CCC1]

R¹ is fluoro and —CH(R^{H})—NR^{I}R^{I′} is

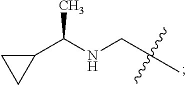 [SMILES: *CN[C@H](C)C1CC1]

R¹ is fluoro and —CH(R^{H})—NR^{I}R^{I′} is

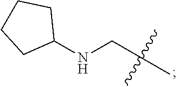 [SMILES: *CNC1CCCC1]

R¹ is fluoro and —CH(R^{H})—NR^{I}R^{I′} is

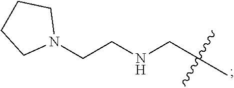 [SMILES: *CNCCN1CCCC1]

R¹ is fluoro and —CH(R^{H})—NR^{I}R^{I′} is

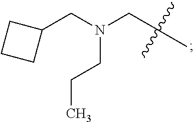 [SMILES: *CN(CCC)CC1CCC1]

R¹ is fluoro and —CH(R^{H})—NR^{I}R^{I′} is

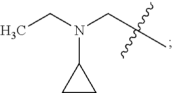 [SMILES: *CN(CC)C1CC1]

R¹ is fluoro and —CH(R^{H})—NR^{I}R^{I′} is

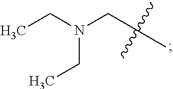 [SMILES: *CN(CC)CC]

R¹ is fluoro and —CH(R^{H})—NR^{I}R^{I′} is

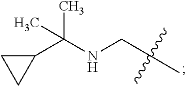 [SMILES: *CNC(C)(C)C1CC1]

R¹ is fluoro and —CH(R^{H})—NR^{I}R^{I′} is

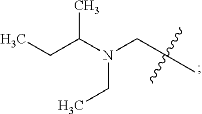 [SMILES: *CN(CC)C(C)CC]

R¹ is fluoro and —CH(R^{H})—NR^{I}R^{I′} is

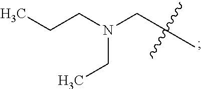 [SMILES: *CN(CC)CCC]

R¹ is fluoro and —CH(R^{H})—NR^{I}R^{I′} is

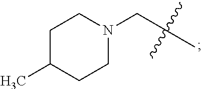 [SMILES: *CN1CCC(C)CC1]

R¹ is fluoro and —CH(R^{H})—NR^{I}R^{I′} is

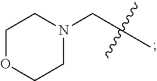 [SMILES: *CN1CCOCC1]

R¹ is fluoro and —CH(R^{H})—NR^{I}R^{I′} is

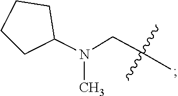 [SMILES: *CN(C)C1CCCC1]

R¹ is fluoro and —CH(R^{H})—NR^{I}R^{I′} is

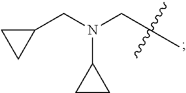 [SMILES: *CN(CC1CC1)C1CC1]

R¹ is fluoro and —CH(R^{H})—NR^{I}R^{I′} is

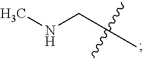 [SMILES: *CNC]

R¹ is fluoro and —CH(R^{H})—NR^{I}R^{I′} is

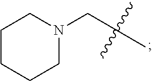 [SMILES: *CN1CCCCC1]

R¹ is fluoro and —CH(R^{H})—NR^{I}R^{I′} is

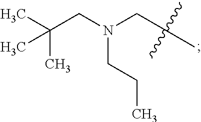 [SMILES: *CN(CCC)CC(C)(C)C]

R¹ is fluoro and —CH(R^{H})—NR^{I}R^{I′} is

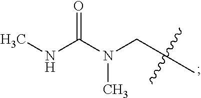 [SMILES: *CN(C)C(=O)NC]

R¹ is fluoro and —CH(R^{H})—NR^{I}R^{I′} is

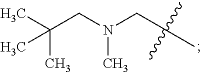 [SMILES: *CN(C)CC(C)(C)C]

R¹ is fluoro and —CH(R^{H})—NR^{I}R^{I′} is

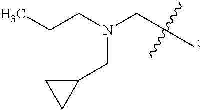 [SMILES: *CN(CCC)CC1CC1]

R¹ is fluoro and —CH(R^{H})—NR^{I}R^{I′} is

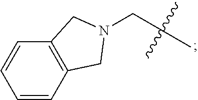 [SMILES: *CN1Cc2ccccc2C1]

R¹ is fluoro and —CH(R^{H})—NR^{I}R^{I′} is

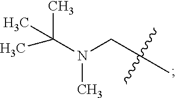 [SMILES: *CN(C)C(C)(C)C]

R¹ is fluoro and —CH(R^{H})—NR^{I}R^{I′} is

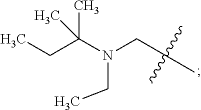 [SMILES: *CN(CC)C(C)(C)CC]

R¹ is fluoro and —CH(R^{H})—NR^{I}R^{I′} is

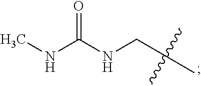 [SMILES: *CNC(=O)NC]

R¹ is fluoro and —CH(R^{H})—NR^{I}R^{I′} is

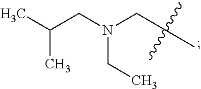 [SMILES: *CN(CC)CC(C)C]

R¹ is fluoro and —CH(R^{H})—NR^{I}R^{I′} is

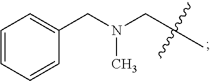 [SMILES: *CN(C)Cc1ccccc1]

R¹ is fluoro and —CH(R^{H})—NR^{I}R^{I′} is

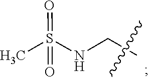

R¹ is fluoro and —CH(R^{H})—NR^{I}R^{I′} is

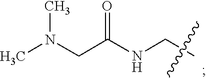

R¹ is fluoro and —CH(R^{H})—NR^{I}R^{I′} is

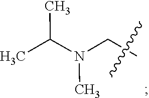

R¹ is fluoro and —CH(R^{H})—NR^{I}R^{I′} is

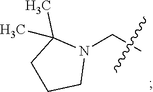

R¹ is fluoro and —CH(R^{H})—NR^{I}R^{I′} is

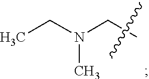

R¹ is fluoro and —CH(R^{H})—NR^{I}R^{I′} is

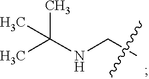

R¹ is fluoro and —CH(R^{H})—NR^{I}R^{I′} is

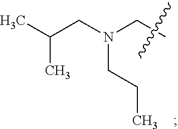

R¹ is fluoro and —CH(R^{H})—NR^{I}R^{I′} is

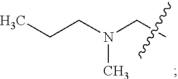

R¹ is fluoro and —CH(R^{H})—NR^{I}R^{I′} is

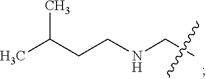

R¹ is fluoro and —CH(R^{H})—NR^{I}R^{I′} is

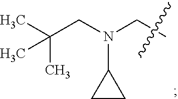

R¹ is fluoro and —CH(R^{H})—NR^{I}R^{I′} is

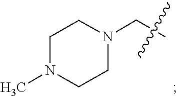

R¹ is fluoro and —CH(R^{H})—NR^{I}R^{I′} is

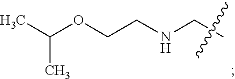

R¹ is fluoro and —CH(R^{H})—NR^{I}R^{I′} is

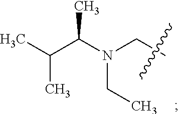

R¹ is fluoro and —CH(R^{H})—NR^{I}R^{I′} is

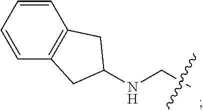

R¹ is fluoro and —CH(R^{H})—NR^{I}R^{I′} is

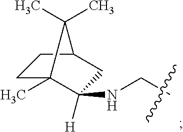

R¹ is fluoro and —CH(R^{H})—NR^{I}R^{I′} is

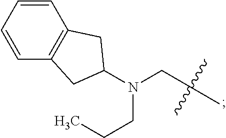 [SMILES: *CN(CCC)C1Cc2ccccc2C1]

R¹ is fluoro and —CH(R^{H})—NR^{I}R^{I′} is

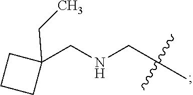 [SMILES: *CNCC1(CC)CCC1]

R¹ is fluoro and —CH(R^{H})—NR^{I}R^{I′} is

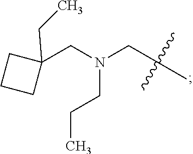 [SMILES: *CN(CCC)CC1(CC)CCC1]

R¹ is fluoro and —CH(R^{H})—NR^{I}R^{I′} is

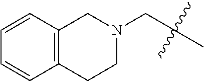 [SMILES: *CN1CCc2ccccc2C1]

R¹ is fluoro and —CH(R^{H})—NR^{I}R^{I′} is

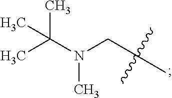 [SMILES: *CN(C)C(C)(C)C]

R¹ is fluoro and —CH(R^{H})—NR^{I}R^{I′} is

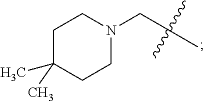 [SMILES: *CN1CCC(C)(C)CC1]

R¹ is fluoro and —CH(R^{H})—NR^{I}R^{I′} is

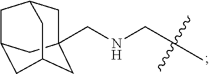 [SMILES: *CNCC12CC3CC(CC(C3)C1)C2]

R¹ is fluoro and —CH(R^{H})—NR^{I}R^{I′} is

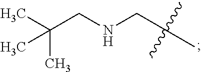 [SMILES: *CNCC(C)(C)C]

R¹ is fluoro and —CH(R^{H})—NR^{I}R^{I′} is

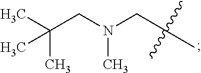 [SMILES: *CN(C)CC(C)(C)C]

R¹ is fluoro and —CH(R^{H})—NR^{I}R^{I′} is

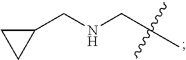 [SMILES: *CNCC1CC1]

R¹ is fluoro and —CH(R^{H})—NR^{I}R^{I′} is

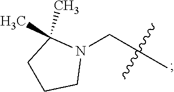

R¹ is fluoro and —CH(R^{H})—NR^{I}R^{I′} is

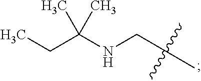 [SMILES: *CNC(C)(C)CC]

R¹ is fluoro and —CH(R^{H})—NR^{I}R^{I′} is

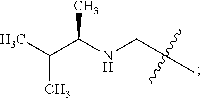 [SMILES: *CN[C@H](C)C(C)C]

R¹ is fluoro and —CH(R^{H})—NR^{I}R^{I′} is

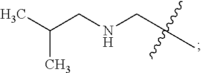 [SMILES: *CNCC(C)C]

and
R¹ is chloro and —CH(R^{H})—NR^{I}R^{I′} is

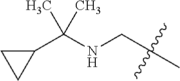 [SMILES: *CNC(C)(C)C1CC1]

or a pharmaceutically acceptable salt of any of the foregoing. The above listed compounds were prepared according to the synthetic procedures detailed in U.S. Pat. No. 9,315,451 incorporated herein by reference in its entirety.

In a fifth aspect of the twenty-fifth embodiment, R¹ is —OCH₃, —CF₃, Cl or F.

A twenty-sixth embodiment of the invention is a compound selected from

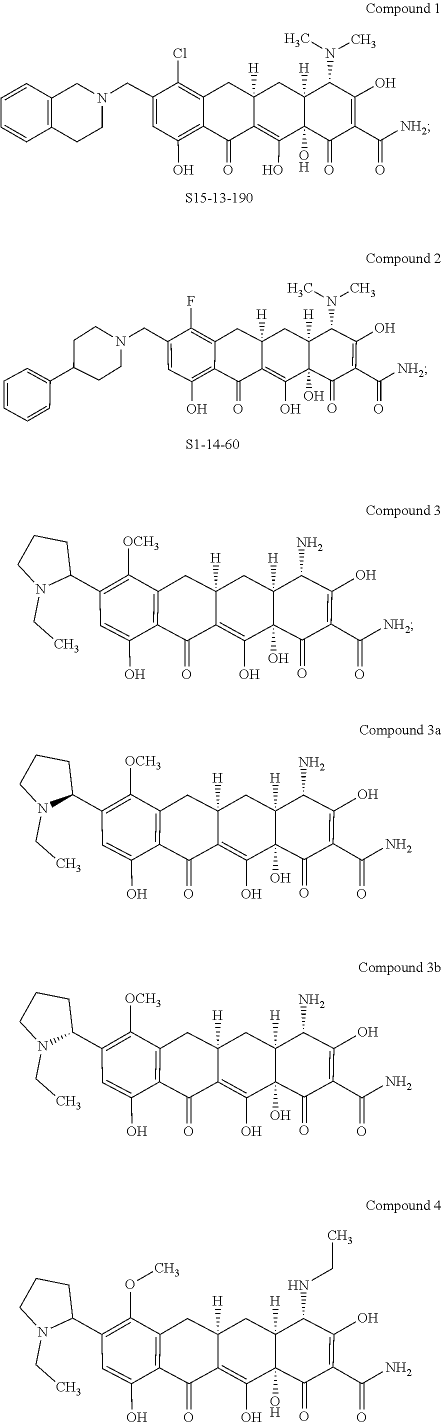

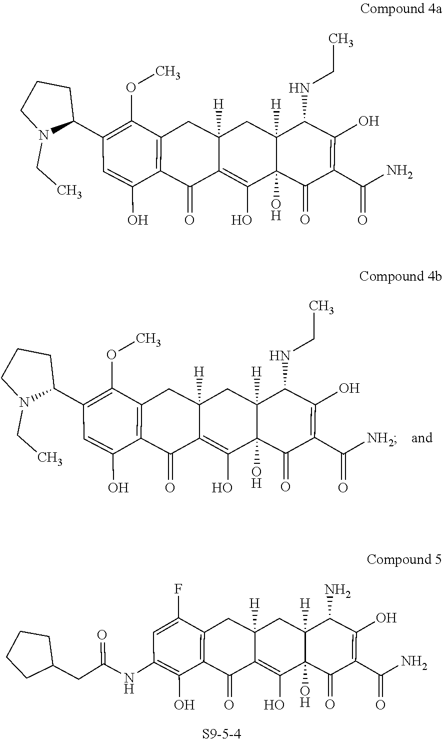 [SMILES: CCN[C@@H]1C(O)=C(C(N)=O)C(=O)[C@@]2(O)C(O)=C3C(=O)c4c(O)cc([C@@H]5CCCN5CC)c(OC)c4C[C@H]3C[C@@H]12 ; CCN[C@@H]1C(O)=C(C(N)=O)C(=O)[C@@]2(O)C(O)=C3C(=O)c4c(O)cc([C@H]5CCCN5CC)c(OC)c4C[C@H]3C[C@@H]12 ; "and" ; NC(=O)C1=C(O)[C@@H](N)[C@@H]2C[C@@H]3Cc4c(F)cc(NC(=O)CC5CCCC5)c(O)c4C(=O)C3=C(O)[C@]2(O)C1=O]

or a pharmaceutically acceptable salt thereof.

### Further Embodiments

In further embodiments, the present invention relates to a method of treating a hematological cancer in a subject in need thereof and compounds for use in treating such cancer. The method comprises administering to the subject an effective amount of a compound represented by any one of structural formulas described below or a pharmaceutically acceptable salt thereof.

A twenty-seventh embodiment, the present invention is a method of treating a hematological cancer comprising administering to a subject in need of treatment an effective amount of a compound having Structural Formula (I) or (I′):

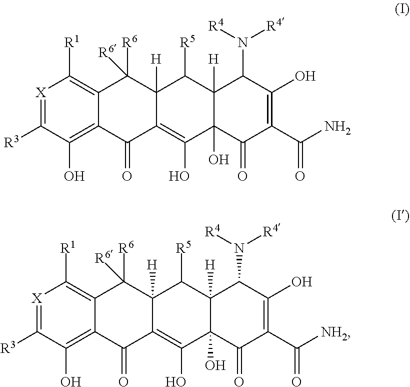

or a pharmaceutically acceptable salt thereof, or a pharmaceutically acceptable composition thereof. In a first aspect of the twenty-sixth embodiment:

X is selected from C(R²) and N;

R¹ is —OR^{A}, hydrogen, halo, —(C₁-C₆ alkyl), —C(O)NR^{B}R^{B′}, —NR^{B}R^{B′}, —S(O)₀₋₂R^{C}, (C₀-C₆ alkylenyl)-(C₃₋₁₂) carbocyclyl, and —(C₀-C₆ alkylenyl)-(4- to 13-member) heterocyclyl;

R² is —(C₀-C₆ alkylenyl)-(4- to 13-member) heterocyclyl, hydrogen, halo, —(C₁-C₆ alkyl), —OR^{A}, —C(O)NR^{B}R^{B′}, —NR^{B}R^{B′}, —S(O)₀₋₂R^{C}, or (C₀-C₆ alkylenyl)-(C₃₋₁₂) carbocyclyl; or

R¹ and R² are optionally taken together with atoms to which they are bound to form a C₃₋₁₂ carbocyclyl or a 4- to 13-member heterocyclyl ring;

each of R³, R⁵ and R⁶ is independently selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylenyl)-(C₃₋₁₂) carbocyclyl, and —(C₀-C₆ alkylenyl)-(4- to 13-member) heterocyclyl; or

R² and R³ are optionally taken together with atoms to which they are bound to form a C₃₋₁₂ carbocyclyl or a 4- to 13-member heterocyclyl ring;

R⁴ is selected from hydrogen, —(C₁-C₆ alkyl), —(C₀-C₆ alkylenyl)-(C₃₋₁₂) carbocyclyl, and —(C₀-C₆ alkylenyl)-(4- to 13-member) heterocyclyl;

R^{4′} is selected from hydrogen, —(C₁-C₆ alkyl), S(O)₁₋₂R^{C}, —(C₀-C₆ alkylenyl)-(C₃₋₁₂) carbocyclyl, —(C₀-C₆ alkylenyl)-(4- to 13-member) heterocyclyl, —C(O)—(C₁-C₆ alkyl), and —C(O)—(C₁-C₆ alkyl)-NR^{D}R^{E}, —C(NR*)NR**R***, wherein R*, R**, and R***, each independently, is H or a C₁₋₄ alkyl, —C(O)—(C₃₋₁₂)carbocyclyl; or

R⁴ and R^{4′} are optionally taken together with the nitrogen atom to which they are commonly bound to form a 4-8 membered ring optionally comprising 1-2 additional heteroatoms independently selected from N, O and S;

R^{6′} is selected from hydrogen, —(C₁-C₆ alkyl) and —(C₃-C₆ cycloalkyl);

each R^{A} is independently selected from —(C₁-C₆ alkyl), hydrogen, —(C₀-C₆ alkylenyl)-(C₃₋₁₂) carbocyclyl, —(C₀-C₆ alkylenyl)-(4- to 13-member) heterocyclyl, —C(O)—(C₁-C₆ alkyl), —C(O)—(C₀-C₆ alkylenyl)-(C₃₋₁₂) carbocyclyl, —C(O)—(C₀-C₆ alkylenyl)-(4- to 13-member) heterocyclyl, and —C(O)N(R^{D})(R^{E});

each R^{B} and each R^{B′} Is independently selected from hydrogen, —(C₁-C₆ alkyl), —(C₁-C₆ haloalkyl), —(C₀-C₆ alkylenyl)-(C₃₋₁₂) carbocyclyl, —(C₀-C₆ alkylenyl)-(4- to 13-member) heterocyclyl, —S(O)₁₋₂—(C₁-C₆ alkyl), —S(O)₁₋₂—(C₀-C₆ alkylenyl)-(C₃₋₁₂) carbocyclyl, —S(O)₁₋₂—(C₀-C₆ alkylenyl)-(4- to 13-member) heterocyclyl, —C(O)—(C₁-C₆ alkyl), —C(O)—(C₀-C₆ alkylenyl)-(C₃₋₁₂) carbocyclyl, —C(O)H, —C(O)—(C₀-C₆ alkylenyl)-(4- to 13-member) heterocyclyl, —C(O)—(C₀-C₆ alkylenyl)-N(R^{D})(R^{E}), and —N⁺(R^{F})₃, wherein R^{F}, for each occurrence independently, is H, a C₁₋₆ alkyl, a C₁₋₆ haloalkyl, a (C₁₋₄ alkoxy)-(C₁₋₆)alkyl, an amino(C₁₋₆)alkyl or a mono- or di(C₁₋₄ alkyl)amino-(C₁₋₆)alkyl, a (C₃₋₁₂)carbocyclyl-(C₀₋₃)alkylenyl, a or any two R^{F}, taken together with the nitrogen atom to which they are attached, for a 4- to 13-member heterocyclyl, optionally including one additional heteroatom selected from O, N or S;

each R^{C} is independently selected from —(C₁-C₆ alkyl), —(C₀-C₆ alkylenyl)-(C₃₋₁₂) carbocyclyl and —(C₀-C₆ alkylenyl)-(4- to 13-member) heterocyclyl; and

each R^{D} and each R^{E} is independently selected from hydrogen, —(C₁-C₆ alkyl), —(C₀-C₆ alkylenyl)-(C₃₋₁₂) carbocyclyl, and —(C₀-C₆ alkylenyl)-(4- to 13-member) heterocyclyl,

wherein:

any alkyl, or alkylenyl portion of R¹, R², R³, R⁴, R^{4′}, R⁵, R⁶ is optionally and independently substituted with one or more substituents independently selected from halo, ═O, OR^{A}, NR^{B}R^{B′}, and S(O)₀₋₂R^{C};

any alkyl or alkylenyl portion of R^{6′}, R^{A}, or R^{C}, is optionally and independently substituted with one or more fluoro;

any carbocyclyl or heterocyclyl portion of any of R¹, R², R³, R⁴, R^{4′}, R⁵, R⁶, or any ring formed by taking together R¹ and R², R² and R³ or R⁴ and R^{4′} is optionally and independently substituted on a carbon atom with one or more substituents independently selected from halo, ═O, C₁-C₄ fluoroalkyl, C₁-C₄ alkyl, —(C₀-C₆ alkylenyl)-(C₃-C₁₀ carbocyclyl), —(C₀-C₆ alkylenyl)-(4-13 membered heterocyclyl), OR^{A}, —(C₀-C₆ alkylenyl)-NR^{B}R^{B′}, and S(O)₀₋₂R^{C};

any heterocyclyl portion of any of R¹, R², R³, R⁴, R^{4′}, R⁵, R⁶, or any ring formed by taking together R¹ and R², R² and R³ or R⁴ and R^{4′} is optionally and independently substituted on a substitutable nitrogen atom with R^{F};

each R^{F} is independently selected from —(C₁-C₆ alkyl), —(C₁-C₆ haloalkyl), —(C₁-C₆ hydroxyalkyl), —(C₀-C₆ alkylenyl)-(C₃₋₁₂) carbocyclyl, —(C₀-C₆ alkylenyl)-(4- to 13-member) heterocyclyl, —S(O)₁₋₂—(C₁-C₆ alkyl), —S(O)₁₋₂—(C₀-C₆ alkylenyl)-(C₃₋₁₂)carbocyclyl, —S(O)₁₋₂—(C₀-C₆ alkylenyl)-(4- to 13-member) heterocyclyl, —C(O)—(C₁-C₆ alkyl), —C(O)—(C₀-C₆ alkylenyl)-(C₃₋₁₂) carbocyclyl, —C(O)H, —C(O)—(C₀-C₆ alkylenyl)-(4- to 13-member) heterocyclyl, —(C₀-C₆ alkylenyl)-C(O)₂—(C₁-C₆ alkyl), —(C₁-C₆ alkylenyl)-NR^{B}R^{B′} and —C(O)N(R^{D})(R^{E});

any carbocyclyl or heterocyclyl portion of R^{A}, R^{B}, R^{B′}, R^{C}, R^{D}, R^{E}, R^{F}, any cycloalkyl portion of R^{6′}, or any substituent of R¹, R², R³, R⁴, R^{4′}, R⁵, R⁶ is optionally and independently substituted on a carbon atom with a one or more substituents independently selected from fluoro, chloro, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂;

any heterocyclyl portion of R^{A}, R^{B}, R^{B′}, R^{C}, R^{D}, R^{E}, R^{F}, or any heterocyclyl substituent of R¹, R², R³, R⁴, R^{4′}, R⁵, or R⁶ is optionally substituted on a substitutable nitrogen atom with —C₁-C₄ alkyl, or —S(O)₁₋₂—(C₁-C₄ alkyl).

In a second aspect of the twenty-sixth embodiment:

X is selected from N and C(R²);

each of R¹, R², R³, R⁵ and R⁶ is independently selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylenyl)-(C₃₋₁₂) carbocyclyl, and —(C₀-C₆ alkylenyl)-(4- to 13-member)heterocyclyl; or

R¹ and R² are optionally taken together with atoms to which they are bound to form a C₃₋₁₂ carbocyclyl or 4- to 13-member heterocyclyl ring; or

R² and R³ are optionally taken together with atoms to which they are bound to form a C₃₋₁₂ carbocyclyl or 4- to 13-member heterocyclyl ring;

R⁴ is selected from hydrogen, —(C₁-C₆ alkyl), —(C₀-C₆ alkylenyl)-(C₃₋₁₂) carbocyclyl, and —(C₀-C₆ alkylenyl)-(4- to 13-member)heterocyclyl;

R^{4′} is selected from hydrogen, —(C₂-C₆ alkyl), S(O)₁₋₂R^{C}, —(C₀-C₆ alkylenyl)-(C₃₋₁₂) carbocyclyl, —(C₀-C₆ alkylenyl)-(4- to 13-member)heterocyclyl, —C(O)—(C₁-C₆ alkyl), and —C(O)—(C₁-C₆ alkyl)-NR^{D}R^{E}; or

R⁴ and R^{4′} are optionally taken together with the nitrogen atom to which they are commonly bound to form a 4-8 membered ring optionally comprising 1-2 additional heteroatoms independently selected from N, O and S;

R^{6′} is selected from hydrogen, —(C₁-C₆ alkyl) and —(C₃-C₆ cycloalkyl);

each R^{A} is independently selected from hydrogen, —(C₁-C₆ alkyl), —(C₀-C₆ alkylenyl)-(C₃₋₁₂) carbocyclyl, —(C₀-C₆ alkylenyl)-(4- to 13-member)heterocyclyl, —C(O)—(C₁-C₆ alkyl), —C(O)—(C₀-C₆ alkylenyl)-(C₃₋₁₂) carbocyclyl, —C(O)—(C₀-C₆ alkylenyl)-(4- to 13-member)heterocyclyl, and —C(O)N(R^{D})(R^{E});

each R^{B} and each R^{B′} is independently selected from hydrogen, —(C₁-C₆ alkyl), —(C₀-C₆ alkylenyl)-(C₃₋₁₂) carbocyclyl, —(C₀-C₆ alkylenyl)-(4- to 13-member)heterocyclyl, —S(O)₁₋₂—(C₁-C₆ alkyl), —S(O)₁₋₂—(C₀-C₆ alkylenyl)-(C₃₋₁₂) carbocyclyl, —S(O)₁₋₂—(C₀-C₆ alkylenyl)-(4- to 13-member)heterocyclyl, —C(O)—(C₁-C₆ alkyl), —C(O)—(C₀-C₆ alkylenyl)-(C₃₋₁₂) carbocyclyl, —C(O)H, —C(O)—(C₀-C₆ alkylenyl)-(4- to 13-member)heterocyclyl, and —C(O)N(R^{D})(R^{E});

each R^{C} is independently selected from —(C₁-C₆ alkyl), —(C₀-C₆ alkylenyl)-(C₃₋₁₂) carbocyclyl and —(C₀-C₆ alkylenyl)-(4- to 13-member)heterocyclyl; and

each R^{D} and each R^{E} is independently selected from hydrogen, —(C₁-C₆ alkyl), —(C₀-C₆ alkylenyl)-(C₃₋₁₂) carbocyclyl, and —(C₀-C₆ alkylenyl)-(4- to 13-member)heterocyclyl,

wherein:

any alkyl, or alkylenyl portion of R¹, R², R³, R⁴, R^{4′}, R⁵, R⁶ is optionally and independently substituted with one or more substituents independently selected from halo, ═O, OR^{A}, NR^{B}R^{B′}, and S(O)₀₋₂R^{C};

any alkyl or alkylenyl portion of R^{6′}, R^{A}, or R^{C}, is optionally and independently substituted with one or more fluoro;

any carbocyclyl or heterocyclyl portion of any of R¹, R², R³, R⁴, R^{4′}, R⁵, R⁶, or any ring formed by taking together R¹ and R², R² and R³, or R⁴ and R^{4′} is optionally and independently substituted on a carbon atom with one or more substituents independently selected from halo, ═O, C₁-C₄ fluoroalkyl, C₁-C₄ alkyl, C₃-C₁₀ carbocyclyl, a 4-13 membered heterocyclyl, OR^{A}, NR^{B}R^{B′}, and S(O)₀₋₂R^{C};

any heterocyclyl portion of any of R¹, R², R³, R⁴, R^{4′}, R⁵, R⁶, or any ring formed by taking together R¹ and R², R² and R³, or R⁴ and R^{4′} is optionally and independently substituted on a substitutable nitrogen atom with R^{F};

each R^{F} is independently selected from —(C₁-C₆ alkyl), —(C₀-C₆ alkylenyl)-(C₃₋₁₂) carbocyclyl, —(C₀-C₆ alkylenyl)-(4- to 13-member)heterocyclyl, —S(O)₁₋₂—(C₁-C₆ alkyl), —S(O)₁₋₂—(C₀-C₆ alkylenyl)-(C₃₋₁₂) carbocyclyl, —S(O)₁₋₂—(C₀-C₆ alkylenyl)-(4- to 13-member)heterocyclyl, —C(O)—(C₁-C₆ alkyl), —C(O)—(C₀-C₆ alkylenyl)-(C₃₋₁₂) carbocyclyl, —C(O)H, —C(O)—(C₀-C₆ alkylenyl)-(4- to 13-member)heterocyclyl, and —C(O)N(R^{D})(R^{E});

any carbocyclyl or heterocyclyl portion of R^{A}, R^{B}, R^{B′}, R^{C}, R^{D}, R^{E}, R^{F}, any cycloalkyl portion of R^{6′}, or any substituent of R¹, R², R³, R⁴, R^{4′}, R⁵, R⁶ is optionally and independently substituted on a carbon atom with a one or more substituents independently selected from fluoro, chloro, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂; and

any heterocyclyl portion of R^{A}, R^{B}, R^{B′}, R^{C}, R^{D}, R^{E}, R^{F}, or any heterocyclyl substituent of R¹, R², R³, R⁴, R^{4′}, R⁵, or R⁶ is optionally substituted on a substitutable nitrogen atom with —C₁-C₄ alkyl, or —S(O)₁₋₂—(C₁-C₄ alkyl). The remainder of the values and example values of the variables in structural formulas (I) and (I′) of the 26^{th} embodiment are as defined above with respect to the first aspect of the 26^{th} embodiment.

In a third aspect of the 26^{th} embodiment, each of R⁵, R⁶ and R^{6′} is hydrogen. The remainder of the values and example values of the variables in structural formulas (I) and (I′) of the 26^{th} embodiment are as defined above with respect to the first and second aspects of the 26^{th} embodiment.

In a fourth aspect of the 26^{th} embodiment, R⁴ is selected from hydrogen and —(C₁-C₆ alkyl); R^{4′} is selected from hydrogen, —(C₂-C₆ alkyl) optionally substituted with one or more substituents independently selected from hydroxy and halo, —(C₃-C₆ cycloalkyl), —C(O)—(C₁-C₆ alkyl), —C(O)—(C₁-C₆ alkylenyl)-N(R^{D})(R^{E}), and S(O)₁₋₂R^{C}; or R⁴ and R^{4′} are taken together with the nitrogen atom to which they are commonly bound to form a 4-6 membered ring optionally comprising 1-2 additional heteroatoms independently selected from N, O and S; R^{C} is —(C₁-C₆ alkyl); and each of R^{D} and R^{E} is independently selected from hydrogen and —(C₁-C₆ alkyl). The remainder of the values and example values of the variables in structural formulas (I) and (I′) of the 26^{th} embodiment are as defined above with respect to the aspects one through three of the 26^{th} embodiment.

In the fifth aspect of the 26^{th} embodiment, R⁴ is selected from hydrogen and —(C₁-C₆ alkyl); R^{4′} is selected from hydrogen, —(C₂-C₆ alkyl), —(C₃-C₆ cycloalkyl), —C(O)—(C₁-C₆ alkyl), —C(O)—(C₁-C₆ alkylenyl)-N(R^{D})(R^{E}), and S(O)₁₋₂R^{C}; R^{C} is —(C₁-C₆ alkyl); and each of R^{D} and R^{E} is independently selected from hydrogen and —(C₁-C₆ alkyl). The remainder of the values and example values of the variables in structural formulas (I) and (I′) of the 26^{th} embodiment are as defined above with respect to the aspects one through four of the 26^{th} embodiment.

In the sixth aspect of the 26^{th} embodiment, R⁴ is selected from hydrogen, methyl, ethyl and propyl; and R^{4′} is selected from hydrogen, ethyl, propyl, cyclopropyl, —C(O)CH₃, —C(O)CH₂N(CH₃)₂, and —S(O)₂CH₃. The remainder of the values and example values of the variables in structural formulas (I) and (I′) of the 26^{th} embodiment are as defined above with respect to the aspects one through five of the 26^{th} embodiment.

In the seventh aspect of the 26^{th} embodiment, R¹ is selected from hydrogen, halo, —(C₁-C₆ alkyl) optionally substituted with one or more substituents independently selected from halo, —NR^{B}R^{B′}, —C(O)NR^{B}R^{B′}, —OR^{A}, —(C₀-C₆ alkylenyl)-(C₃₋₁₂) carbocyclyl, and —(C₀-C₆ alkylenyl)-(4- to 13-member) heterocyclyl, wherein R^{A} is C₁-C₆ alkyl optionally substituted with one or more fluoro. The remainder of the values and example values of the variables in structural formulas (I) and (I′) of the 26^{th} embodiment are as defined above with respect to the aspects one through six of the 26^{th} embodiment.

In the eight aspect of the 26^{th} embodiment, R³ is selected from hydrogen and —N(R^{B})(R^{B′}), wherein R^{B} is hydrogen. The remainder of the values and example values of the variables in structural formulas (I) and (I′) of the 26^{th} embodiment are as defined above with respect to the aspects one through seven of the 26^{th} embodiment.

In the ninth aspect of the 26^{th} embodiment, X is C(R²). The remainder of the values and example values of the variables in structural formulas (I) and (I′) of the 26^{th} embodiment are as defined above with respect to the aspects one through eight of the 26^{th} embodiment.

In the tenth aspect of the 26^{th} embodiment, X is C(R²); and R¹ is selected from hydrogen, halo, —(C₁-C₆ alkyl) optionally substituted with one or more substituents independently selected from halo, —NR^{B}R^{B′}, —C(O)NR^{B}R^{B′}, —OR^{A}, —(C₀-C₆ alkylenyl)-(C₃₋₁₂) carbocyclyl, and —(C₀-C₆ alkylenyl)-(4- to 13-member) heterocyclyl, wherein R^{A} is C₁-C₆ alkyl optionally substituted with one or more fluoro. The remainder of the values and example values of the variables in structural formulas (I) and (I′) of the 26^{th} embodiment are as defined above with respect to the aspects one through eight of the 26^{th} embodiment.

In the tenth aspect of the 26^{th} embodiment, R¹ is selected from hydrogen, halo, —(C₁-C₆ alkyl) optionally substituted with one or more substituents independently selected from halo, and —OR^{A}, wherein R^{A} is C₁-C₆ alkyl optionally substituted with one or more fluoro. The remainder of the values and example values of the variables in structural formulas (I) and (I′) of the 26^{th} embodiment are as defined above with respect to the aspects one through nine of the 26^{th} embodiment.

In the eleventh aspect of the 26^{th} embodiment, R¹ is selected from hydrogen, fluoro, chloro, CF₃, OCH₃, OCF₃, N(CH₃)₂ and NHCH₃, for example, R¹ is selected from hydrogen, fluoro, chloro, CF₃ and OCF₃. The remainder of the values and example values of the variables in structural formulas (I) and (I′) of the 26^{th} embodiment are as defined above with respect to the aspects one through ten of the 26^{th} embodiment.

In the twelfth aspect of the 26^{th} embodiment, X is C(R²); and R¹ and R² are taken together with the atoms to which they are bound to form a 4- to 13-member nitrogen-containing heterocyclyl ring, wherein the ring comprising R¹ and R² is optionally substituted on any substitutable nitrogen atom with C₁-C₄ alkyl; and optionally substituted on a carbon atom with NR^{B}R^{B′}, wherein each of R^{B} and R^{B′} is independently selected from hydrogen and C₁-C₆ alkyl. The remainder of the values and example values of the variables in structural formulas (I) and (I′) of the 26^{th} embodiment are as defined above with respect to the aspects one through eleven of the 26^{th} embodiment.

In the thirteenth aspect of the 26^{th} embodiment, X is C(R²); and R¹ and R² are taken together with the carbon atoms to which they are bound to form:

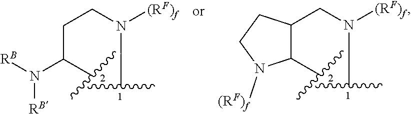

wherein “ [symbol] 1” represents a point of attachment to the carbon atom bound to R¹; and “ [symbol] 2” represents a point of attachment to the carbon atom bound to R²; and f is 0 or 1. For example, R¹ and R² are taken together with the carbon atoms to which they are bound to form:

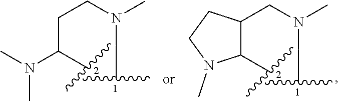

wherein “ [symbol] 1” represents a point of attachment to the carbon atom bound to R¹ and “ [symbol] 2” represents a point of attachment to the carbon atom bound to R². The remainder of the values and example values of the variables in structural formulas (I) and (I′) of the 26^{th} embodiment are as defined above with respect to the aspects one through twelve of the 26^{th} embodiment.

In the fourteenth aspect of the 26^{th} embodiment, X is C(R²); and R² is —(C₀-C₆ alkylenyl)-(4- to 13-member) heterocyclyl optionally substituted on a nitrogen atom with —(C₁-C₆ alkyl); —(C₀-C₆ alkylenyl)-(C₃₋₁₂) carbocyclyl; or —(C₁-C₆)alkyl substituted with NR^{B}R^{B′}. For example, R² is pyrrolidinyl optionally substituted on a nitrogen atom with C₁-C₄ alkyl or benzyl. The remainder of the values and example values of the variables in structural formulas (I) and (I′) of the 26^{th} embodiment are as defined above with respect to the aspects one through eleven of the 26^{th} embodiment.

In the fifteenth aspect of the 26^{th} embodiment, X is C(R²); and R² and R³ are taken together with the atoms to which they are bound to form a nitrogen-containing 4- to 13-member heterocyclyl. For example, R² and R³ are taken together with the atoms to which they are bound to form

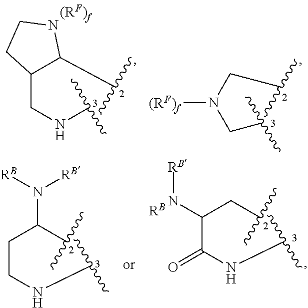

wherein “ [symbol] 2” represents a point of attachment to the carbon atom bound to R²; “ [symbol] 3” represents a point of attachment to the carbon atom bound to R³; and f is 0 or 1. The remainder of the values and example values of the variables in structural formulas (I) and (I′) of the 26^{th} embodiment are as defined above with respect to the aspects one through eleven of the 26^{th} embodiment.

In the sixteenth aspect of the 26^{th} embodiment, X is C(R²); and R³ is selected from hydrogen and —N(R^{B})(R^{B′}), wherein R^{B} is hydrogen and R^{B′} is —C(O)—(C₀-C₆ alkylenyl)-(4- to 13-member) heterocyclyl or —C(O)—(C₀-C₆ alkylenyl)-N(R^{D})(R^{E}). For example, R³ is selected from hydrogen and

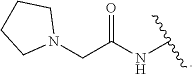 [SMILES: *NC(=O)CN1CCCC1]

The remainder of the values and example values of the variables in structural formulas (I) and (I′) of the 26^{th} embodiment are as defined above with respect to the aspects one through fourteen of the 26^{th} embodiment.

In the seventeenth aspect of the 26^{th} embodiment, X is C(R²). The remainder of the values and example values of the variables in structural formulas (I) and (I′) of the 26^{th} embodiment are as defined above with respect to the aspects one through nine of the 26^{th} embodiment.

In the eighteenth aspect of the 26^{th} embodiment, the compound is represented by any one of the following structural formulas, or a pharmaceutically acceptable salt thereof:

| Compound No. | Compound Structure |
|---|---|
| S3-7-1-A (diastereomer A) S3-7-1-B (diastereomer B) | 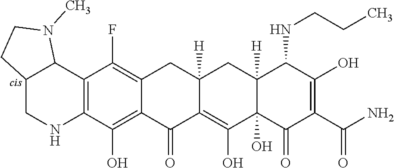 [SMILES: CCCN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-2 | 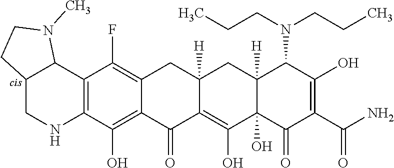 [SMILES: CCCN(CCC)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-3-A (diastereomer A) S3-7-3-B (diastereomer B) | 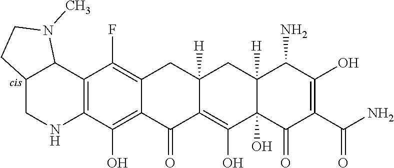 [SMILES: CN1CCC2CNc3c(O)c4c(c(F)c3C21)C[C@H]1C[C@H]2[C@H](N)C(O)=C(C(N)=O)C(=O)[C@@]2(O)C(O)=C1C4=O] |
| S3-7-4-A (diastereomer A) S3-7-4-B (diastereomer B) | 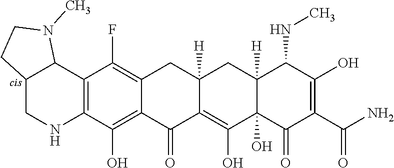 [SMILES: CN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-5 | 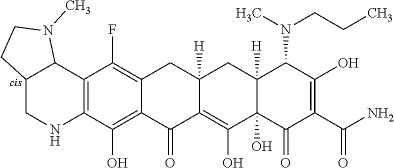 [SMILES: CCCN(C)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-6-A (diastereomer A) S3-7-6-B (diastereomer B) | 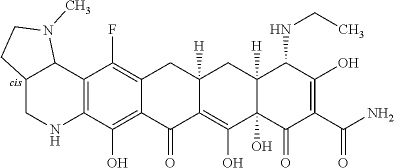 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-7-A (diastereomer A) S3-7-7-B (diastereomer B) | 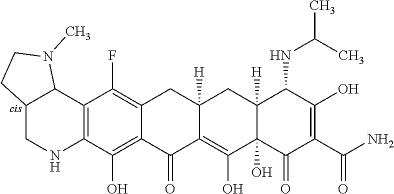 [SMILES: CC(C)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-8-A (diastereomer A) S3-7-8-B (diastereomer B) | 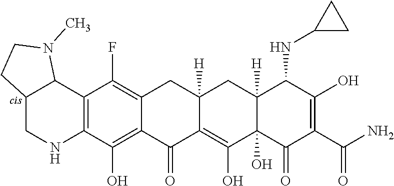 [SMILES: CN1CCC2CNc3c(O)c4c(c(F)c3C21)C[C@H]1C[C@H]2[C@H](NC3CC3)C(O)=C(C(N)=O)C(=O)[C@@]2(O)C(O)=C1C4=O] |
| S3-7-9-A (diastereomer A) S3-7-9-B (diastereomer B) | 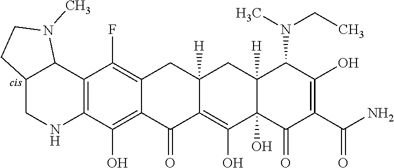 [SMILES: CCN(C)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-10-A (diastereomer A) S3-7-10-B (diastereomer B) | 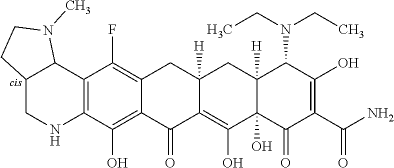 [SMILES: CCN(CC)[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-11 | 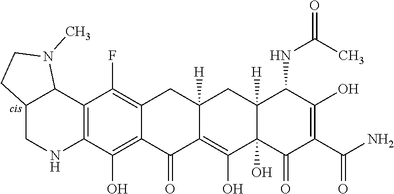 [SMILES: CC(=O)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S3-7-12 | 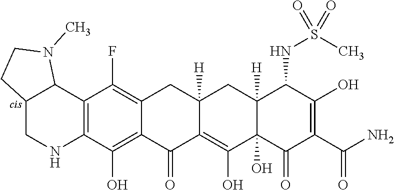 [SMILES: CN1CCC2CNc3c(O)c4c(c(F)c3C21)C[C@H]1C[C@H]2[C@H](NS(C)(=O)=O)C(O)=C(C(N)=O)C(=O)[C@@]2(O)C(O)=C1C4=O] |
| S3-7-13-A (diastereomer A) S3-7-13-B (diastereomer B) | 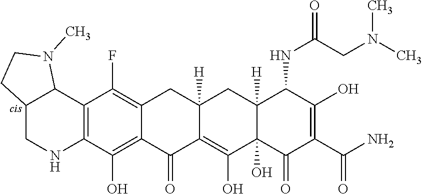 [SMILES: CN(C)CC(=O)N[C@@H]1C(O)=C(C(N)=O)C(=O)[C@@]2(O)C(O)=C3C(=O)c4c(O)c5c(c(F)c4C[C@H]3C[C@@H]12)C1C(CCN1C)CN5] |
| S4-14-1 (diastereomer A) | 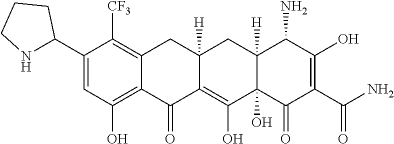 [SMILES: NC(=O)C1=C(O)[C@@H](N)[C@@H]2C[C@@H]3Cc4c(c(O)cc(C5CCCN5)c4C(F)(F)F)C(=O)C3=C(O)[C@]2(O)C1=O] |
| S4-14-2 (diastereomer A) | 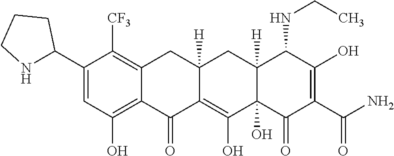 [SMILES: CCN[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-3 (diastereomer A) | 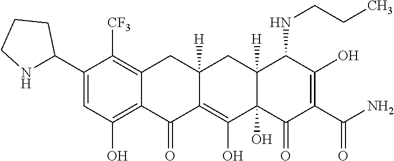 [SMILES: CCCN[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-4 (diastereomer A) | 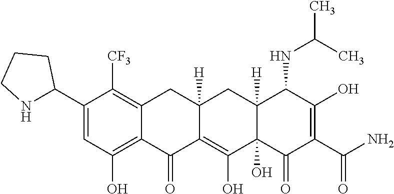 [SMILES: CC(C)N[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-5-A (diastereomer A) S4-14-5-B (diastereomer B) | 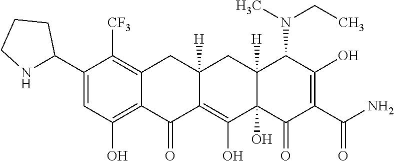 [SMILES: CCN(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-7 (diastereomer A) | 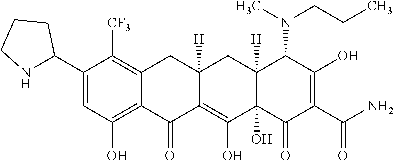 [SMILES: CCCN(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-8 (diastereomer A) | 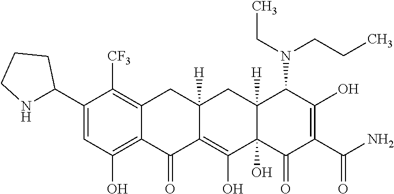 [SMILES: CCCN(CC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-9 (diastereomer A) | 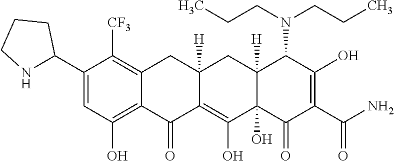 [SMILES: CCCN(CCC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-10 (diastereomer A) | 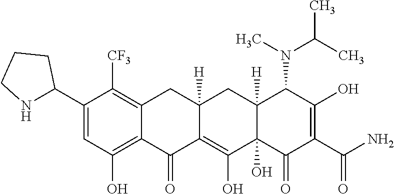 [SMILES: CC(C)N(C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-11 (diastereomer A) | 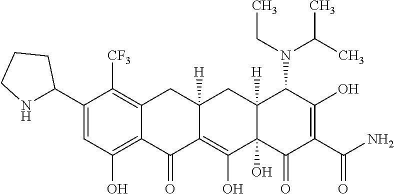 [SMILES: CCN(C(C)C)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-12 (diastereomer A) | 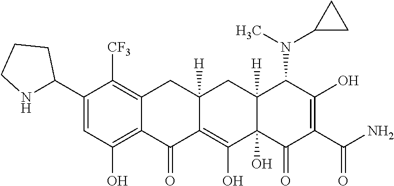 [SMILES: CN(C1CC1)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-13 (diastereomer A) | 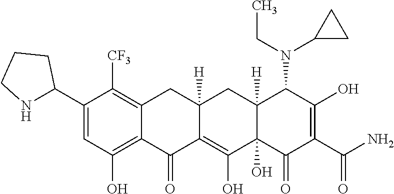 [SMILES: CCN(C1CC1)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-14-A (diastereomer A) S4-14-14-B (diastereomer B) | 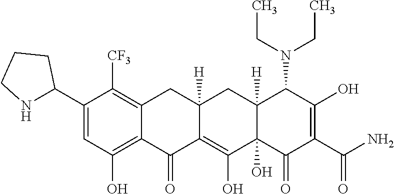 [SMILES: CCN(CC)[C@@H]1C(O)=C(C(N)=O)C(=O)[C@@]2(O)C(O)=C3C(=O)c4c(O)cc(C5CCCN5)c(C(F)(F)F)c4C[C@H]3C[C@@H]12] |
| S4-14-16 (diastereomer A) | 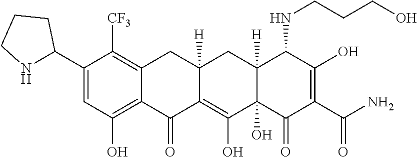 [SMILES: NC(=O)C1=C(O)[C@@H](NCCCO)[C@@H]2C[C@@H]3Cc4c(c(O)cc(C5CCCN5)c4C(F)(F)F)C(=O)C3=C(O)[C@]2(O)C1=O] |
| S4-14-17 (diastereomer A) | 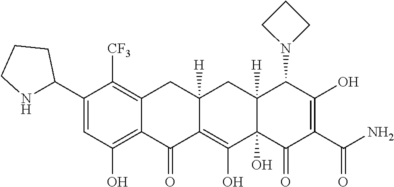 [SMILES: NC(=O)C1=C(O)[C@@H](N2CCC2)[C@@H]2C[C@@H]3Cc4c(c(O)cc(C5CCCN5)c4C(F)(F)F)C(=O)C3=C(O)[C@]2(O)C1=O] |
| S4-14-18 (diastereomer A) | 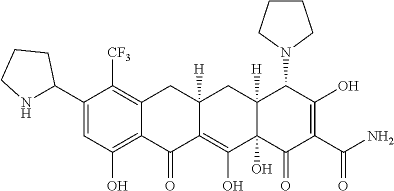 [SMILES: NC(=O)C1=C(O)[C@@H](N2CCCC2)[C@@H]2C[C@@H]3Cc4c(c(O)cc(C5CCCN5)c4C(F)(F)F)C(=O)C3=C(O)[C@]2(O)C1=O] |
| S5-10-1-A (diastereomer A) S5-10-1-B (diastereomer B) | 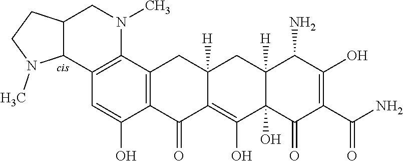 [SMILES: CN1CC2CCN(C)C2c2cc(O)c3c(c21)C[C@H]1C[C@H]2[C@H](N)C(O)=C(C(N)=O)C(=O)[C@@]2(O)C(O)=C1C3=O] |
| S5-10-1-2-A (diastereomer A) S5-10-1-2-B (diastereomer B) | 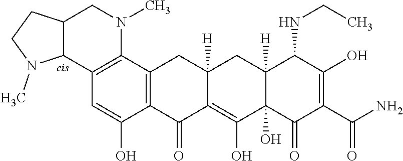 [SMILES: CCN[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S5-10-3-A (diastereomer A) S5-10-3-B (diastereomer B) | 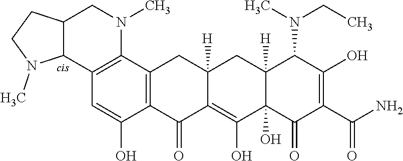 [SMILES: CCN(C)[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S5-10-4-A (diastereomer A) S5-10-4-B (diastereomer B) | 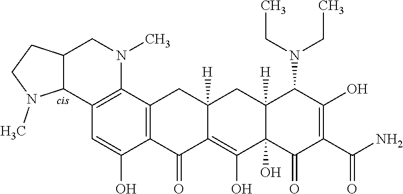 [SMILES: CCN(CC)[C@@H]1C(O)=C(C(N)=O)C(=O)[C@@]2(O)C(O)=C3C(=O)c4c(O)cc5c(c4C[C@H]3C[C@@H]12)N(C)CC1CCN(C)C51] |
| S6-6-1 (single diastereomer) | 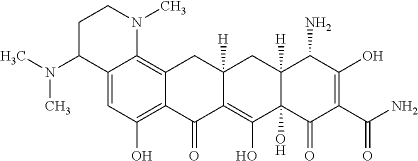 [SMILES: CN1CCC(N(C)C)c2cc(O)c3c(c21)C[C@H]1C[C@H]2[C@H](N)C(O)=C(C(N)=O)C(=O)[C@@]2(O)C(O)=C1C3=O] |
| S6-6-2 (single diastereomer) | 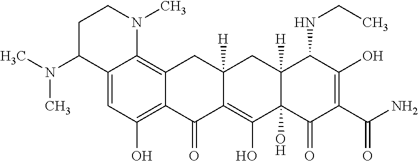 [SMILES: CCN[C@@H]1C(O)=C(C(N)=O)C(=O)[C@@]2(O)C(O)=C3C(=O)c4c(O)cc5c(c4C[C@H]3C[C@@H]12)N(C)CCC5N(C)C] |
| S6-6-3 (single diastereomer) | 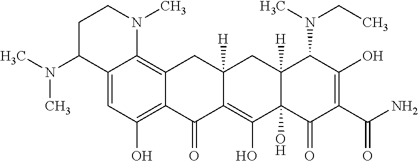 [SMILES: CCN(C)[C@@H]1C(O)=C(C(N)=O)C(=O)[C@@]2(O)C(O)=C3C(=O)c4c(O)cc5c(c4C[C@H]3C[C@@H]12)N(C)CCC5N(C)C] |
| S7-14-1-A (diastereomer A) S7-14-1-B (diastereomer B) | 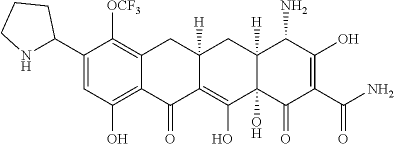 [SMILES: NC(=O)C1=C(O)[C@@H](N)[C@@H]2C[C@@H]3Cc4c(OC(F)(F)F)c(C5CCCN5)cc(O)c4C(=O)C3=C(O)[C@]2(O)C1=O] |
| S7-14-2-A (diastereomer A) | 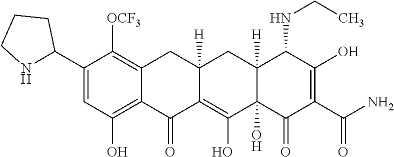 [SMILES: CCN[C@@H]1C(O)=C(C(N)=O)C(=O)[C@@]2(O)C(O)=C3C(=O)c4c(O)cc(C5CCCN5)c(OC(F)(F)F)c4C[C@H]3C[C@@H]12] |
| S7-14-3-A (diastereomer A) | 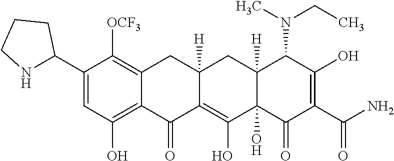 [SMILES: CCN(C)[C@@H]1C(O)=C(C(N)=O)C(=O)[C@@]2(O)C(O)=C3C(=O)c4c(O)cc(C5CCCN5)c(OC(F)(F)F)c4C[C@H]3C[C@@H]12] |
| S8-4-1 | 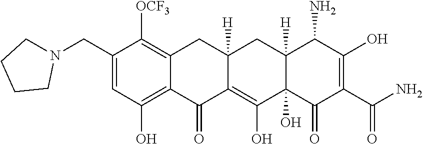 [SMILES: NC(=O)C1=C(O)[C@@H](N)[C@@H]2C[C@@H]3Cc4c(OC(F)(F)F)c(CN5CCCC5)cc(O)c4C(=O)C3=C(O)[C@]2(O)C1=O] |
| S8-4-2 | 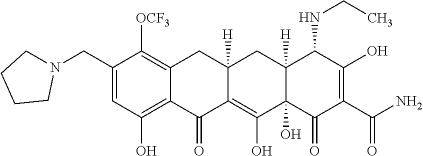 [SMILES: CCN[C@@H]1C(O)=C(C(N)=O)C(=O)[C@@]2(O)C(O)=C3C(=O)c4c(O)cc(CN5CCCC5)c(OC(F)(F)F)c4C[C@H]3C[C@@H]12] |
| S8-4-3 | 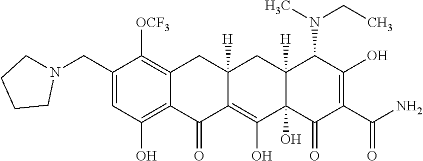 [SMILES: CCN(C)[C@@H]1C(O)=C(C(N)=O)C(=O)[C@@]2(O)C(O)=C3C(=O)c4c(O)cc(CN5CCCC5)c(OC(F)(F)F)c4C[C@H]3C[C@@H]12] |
| S9-4-1 | 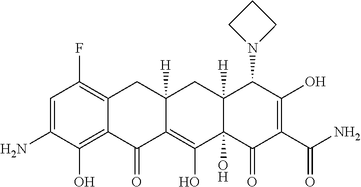 [SMILES: NC(=O)C1=C(O)[C@@H](N2CCC2)[C@@H]2C[C@@H]3Cc4c(F)cc(N)c(O)c4C(=O)C3=C(O)[C@]2(O)C1=O] |
| S9-5-1 | 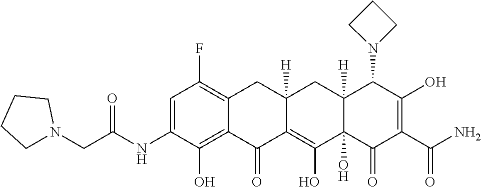 [SMILES: NC(=O)C1=C(O)[C@@H](N2CCC2)[C@@H]2C[C@@H]3Cc4c(F)cc(NC(=O)CN5CCCC5)c(O)c4C(=O)C3=C(O)[C@]2(O)C1=O] |
| S9-5-2 | 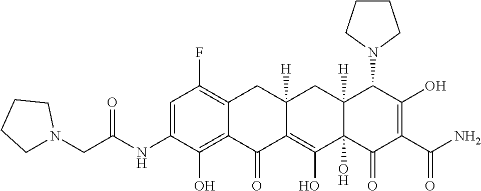 [SMILES: NC(=O)C1=C(O)[C@@H](N2CCCC2)[C@@H]2C[C@@H]3Cc4c(F)cc(NC(=O)CN5CCCC5)c(O)c4C(=O)C3=C(O)[C@]2(O)C1=O] |
| S9-5-3 | 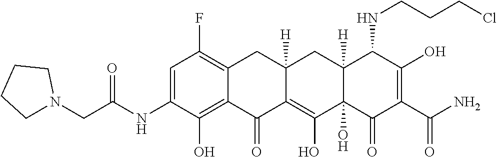 [SMILES: NC(=O)C1=C(O)[C@@H](NCCCCl)[C@@H]2C[C@@H]3Cc4c(F)cc(NC(=O)CN5CCCC5)c(O)c4C(=O)C3=C(O)[C@]2(O)C1=O] |
| S9-5-4 | 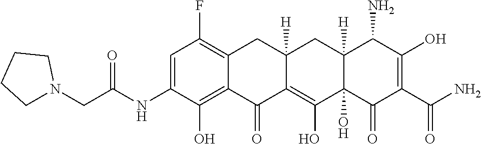 [SMILES: NC(=O)C1=C(O)[C@@H](N)[C@@H]2C[C@@H]3Cc4c(F)cc(NC(=O)CN5CCCC5)c(O)c4C(=O)C3=C(O)[C@]2(O)C1=O] |
| S9-5-5 | 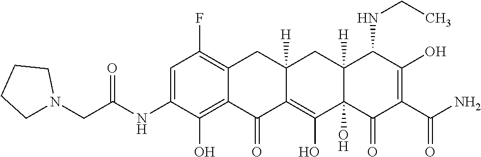 [SMILES: CCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12] |
| S9-5-6 | 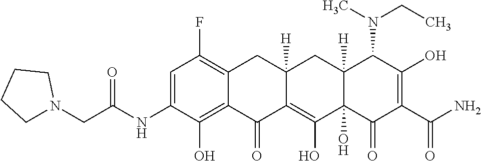 [SMILES: CCN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12] |
| S10-4-1 (single diastereomer) | 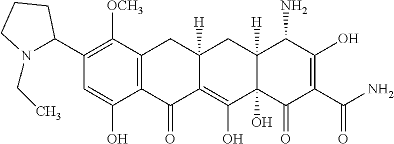 [SMILES: CCN1CCCC1c1cc(O)c2c(c1OC)C[C@H]1C[C@H]3[C@H](N)C(O)=C(C(N)=O)C(=O)[C@@]3(O)C(O)=C1C2=O] |
| S10-4-2 (single diastereomer) | 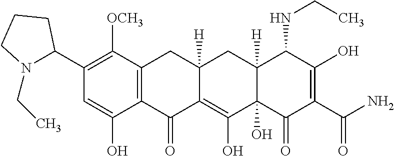 [SMILES: CCN[C@@H]1C(O)=C(C(N)=O)C(=O)[C@@]2(O)C(O)=C3C(=O)c4c(O)cc(C5CCCN5CC)c(OC)c4C[C@H]3C[C@@H]12] |
| S10-4-3 (single diastereomer) | 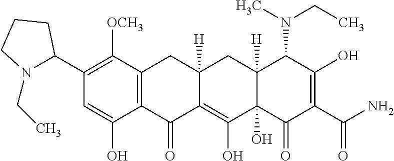 [SMILES: CCN1CCCC1c1cc(O)c2c(c1OC)C[C@H]1C[C@H]3[C@H](N(C)CC)C(O)=C(C(N)=O)C(=O)[C@@]3(O)C(O)=C1C2=O] |
| S11-3-1 | 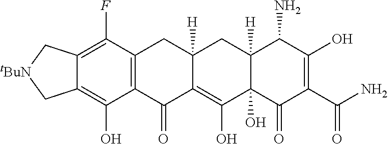 |
| S11-3-2 | 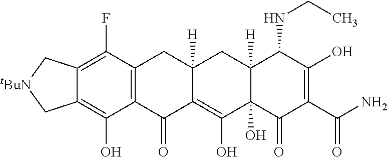 |
| S11-3-3 | 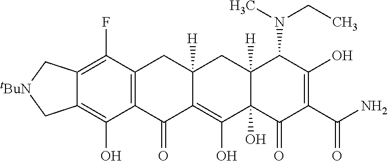 |
| S12-8-1-A (diastereomer A) S12-8-1-B (diastereomer B) | 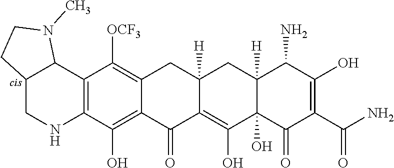 [SMILES: CN1CCC2CNc3c(O)c4c(c(OC(F)(F)F)c3C21)C[C@H]1C[C@H]2[C@H](N)C(O)=C(C(N)=O)C(=O)[C@@]2(O)C(O)=C1C4=O] |
| S12-8-2-A (diastereomer A) | 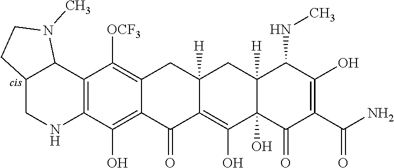 [SMILES: CN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-3-A (diastereomer A) S12-8-3-B) (diastereomer B) | 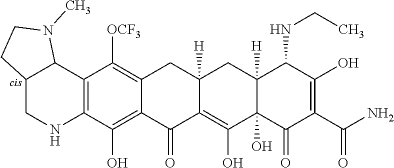 [SMILES: CCN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-4-A (diastereomer A) | 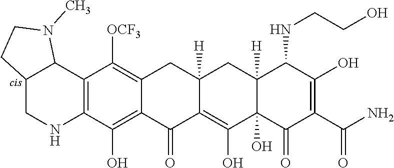 [SMILES: CN1CCC2CNc3c(O)c4c(c(OC(F)(F)F)c3C21)C[C@H]1C[C@H]2[C@H](NCCO)C(O)=C(C(N)=O)C(=O)[C@@]2(O)C(O)=C1C4=O] |
| S12-8-5-A (diastereomer A) | 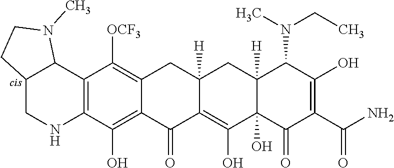 [SMILES: CCN(C)[C@@H]1C(O)=C(C(N)=O)C(=O)[C@@]2(O)C(O)=C3C(=O)c4c(O)c5c(c(OC(F)(F)F)c4C[C@H]3C[C@@H]12)C1C(CCN1C)CN5] |
| S12-8-6-A (diastereomer A) S12-8-6-B (diastereomer B) | 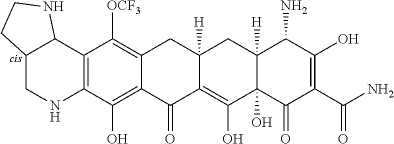 [SMILES: NC(=O)C1=C(O)[C@@H](N)[C@@H]2C[C@@H]3Cc4c(OC(F)(F)F)c5c(c(O)c4C(=O)C3=C(O)[C@]2(O)C1=O)NCC1CCNC51] |
| S12-8-7-A (diastereomer A) | 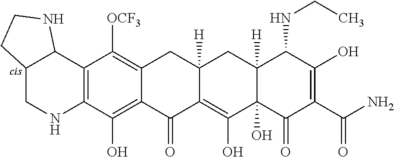 [SMILES: CCN[C@@H]1C(O)=C(C(N)=O)C(=O)[C@@]2(O)C(O)=C3C(=O)c4c(O)c5c(c(OC(F)(F)F)c4C[C@H]3C[C@@H]12)C1NCCC1CN5] |
| S12-8-8-A (diastereomer A) | 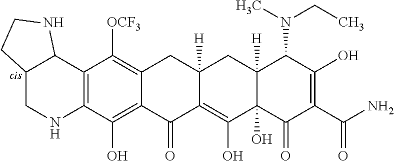 [SMILES: CCN(C)[C@@H]1C(O)=C(C(N)=O)C(=O)[C@@]2(O)C(O)=C3C(=O)c4c(O)c5c(c(OC(F)(F)F)c4C[C@H]3C[C@@H]12)C1NCCC1CN5] |
| S13-5-1 | 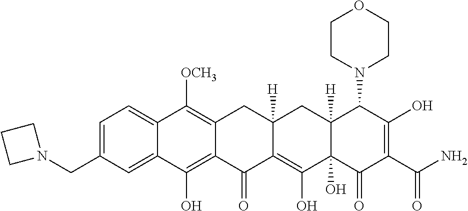 [SMILES: COc1c2c(c(O)c3cc(CN4CCC4)ccc13)C(=O)C1=C(O)[C@]3(O)C(=O)C(C(N)=O)=C(O)[C@@H](N4CCOCC4)[C@@H]3C[C@@H]1C2] |
| S13-5-2 | 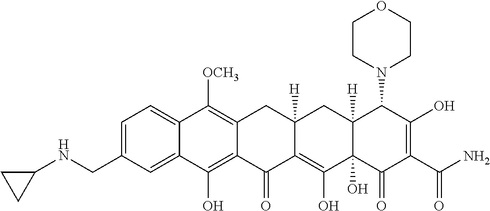 [SMILES: COc1c2c(c(O)c3cc(CNC4CC4)ccc13)C(=O)C1=C(O)[C@]3(O)C(=O)C(C(N)=O)=C(O)[C@@H](N4CCOCC4)[C@@H]3C[C@@H]1C2] |
| S14-8-1 | 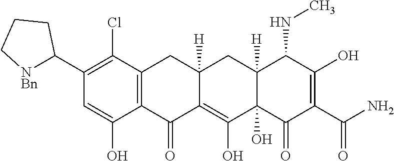 [SMILES: CN[C@@H]1C(O)=C(C(N)=O)C(=O)[C@@]2(O)C(O)=C3C(=O)c4c(O)cc(C5CCCN5Cc5ccccc5)c(Cl)c4C[C@H]3C[C@@H]12] |
| S14-8-2 | 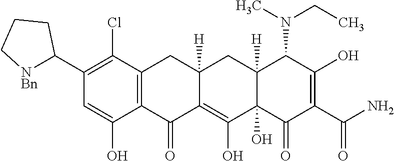 [SMILES: CCN(C)[C@@H]1C(O)=C(C(N)=O)C(=O)[C@@]2(O)C(O)=C3C(=O)c4c(O)cc(C5CCCN5Cc5ccccc5)c(Cl)c4C[C@H]3C[C@@H]12] |
| S14-8-3-A (diastereomer A) S14-8-3-B (diastereomer B) | 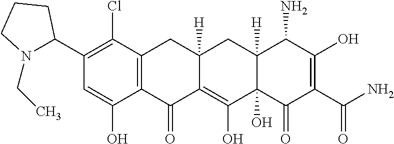 [SMILES: CCN1CCCC1c1cc(O)c2c(c1Cl)C[C@H]1C[C@H]3[C@H](N)C(O)=C(C(N)=O)C(=O)[C@@]3(O)C(O)=C1C2=O] |
| S15-10-1 | 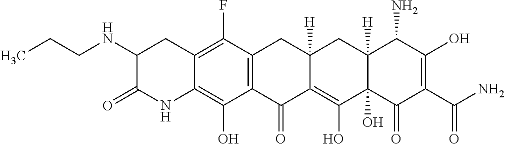 [SMILES: CCCNC1Cc2c(F)c3c(c(O)c2NC1=O)C(=O)C1=C(O)[C@]2(O)C(=O)C(C(N)=O)=C(O)[C@@H](N)[C@@H]2C[C@@H]1C3] |
| S15-10-2 | 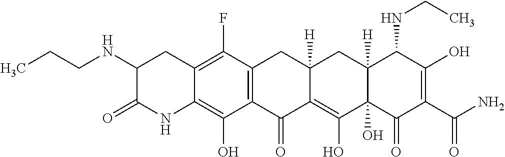 [SMILES: CCCNC1Cc2c(F)c3c(c(O)c2NC1=O)C(=O)C1=C(O)[C@]2(O)C(=O)C(C(N)=O)=C(O)[C@@H](NCC)[C@@H]2C[C@@H]1C3] |
| S15-10-3-A (diastereomer A) S15-10-3-B (diastereomer B) | 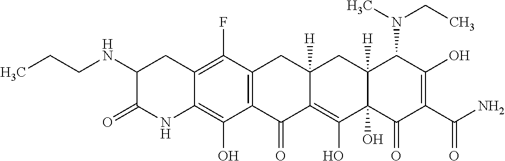 [SMILES: CCCNC1Cc2c(F)c3c(c(O)c2NC1=O)C(=O)C1=C(O)[C@]2(O)C(=O)C(C(N)=O)=C(O)[C@@H](N(C)CC)[C@@H]2C[C@@H]1C3] |
| S16-7-1 (single diastereomer) | 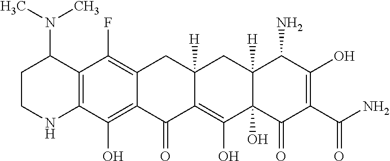 [SMILES: CN(C)C1CCNc2c(O)c3c(c(F)c21)C[C@H]1C[C@H]2[C@H](N)C(O)=C(C(N)=O)C(=O)[C@@]2(O)C(O)=C1C3=O] |
| S16-7-2 (single diastereomer) | 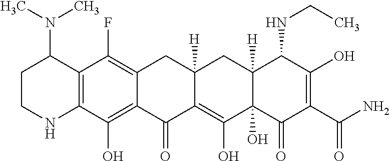 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S16-7-3 (single diastereomer) | 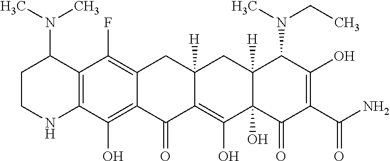 [SMILES: CCN(C)[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S16-7-4 (single diastereomer) | 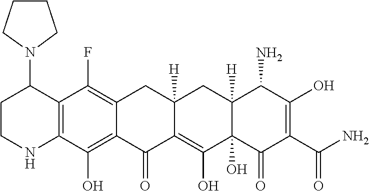 [SMILES: NC(=O)C1=C(O)[C@@H](N)[C@@H]2C[C@@H]3Cc4c(F)c5c(c(O)c4C(=O)C3=C(O)[C@]2(O)C1=O)NCCC5N1CCCC1] |
| S16-7-5 (single diastereomer) | 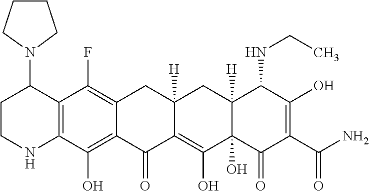 [SMILES: CCN[C@@H]1C(O)=C(C(N)=O)C(=O)[C@@]2(O)C(O)=C3C(=O)c4c(O)c5c(c(F)c4C[C@H]3C[C@@H]12)C(N1CCCC1)CCN5] |
| S16-7-6 (single diastereomer) | 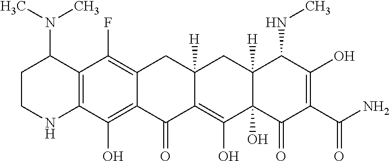 [SMILES: CN[C@@H]1C(O)=C(C(N)=O)C(=O)[C@@]2(O)C(O)=C3C(=O)c4c(O)c5c(c(F)c4C[C@H]3C[C@@H]12)C(N(C)C)CCN5] |
| S17-3-1 | 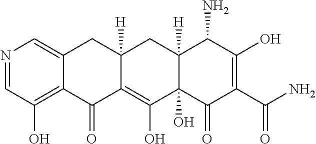 [SMILES: NC(=O)C1=C(O)[C@@H](N)[C@@H]2C[C@@H]3Cc4cncc(O)c4C(=O)C3=C(O)[C@]2(O)C1=O] |
| S17-3-2 | 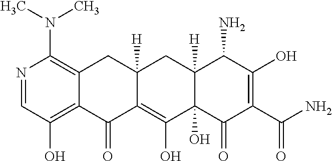 [SMILES: CN(C)c1ncc(O)c2c1C[C@H]1C[C@H]3[C@H](N)C(O)=C(C(N)=O)C(=O)[C@@]3(O)C(O)=C1C2=O] |
| S17-3-3 | 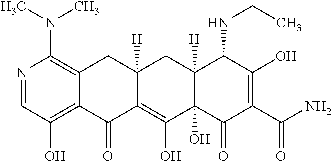 [SMILES: CCN[C@@H]1C(O)=C(C(N)=O)C(=O)[C@@]2(O)C(O)=C3C(=O)c4c(O)cnc(N(C)C)c4C[C@H]3C[C@@H]12] |
| S17-3-4 | 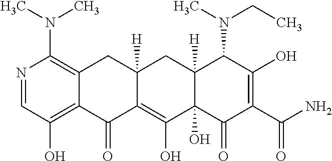 [SMILES: CCN(C)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-5 | 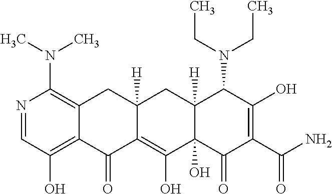 [SMILES: CCN(CC)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-6 | 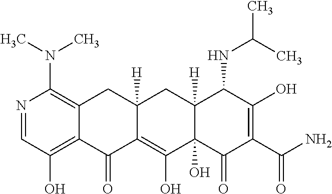 [SMILES: CC(C)N[C@@H]1C(O)=C(C(N)=O)C(=O)[C@@]2(O)C(O)=C3C(=O)c4c(O)cnc(N(C)C)c4C[C@H]3C[C@@H]12] |
| S17-3-7 | 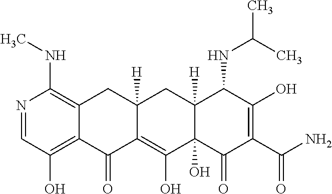 [SMILES: CNc1ncc(O)c2c1C[C@H]1C[C@H]3[C@H](NC(C)C)C(O)=C(C(N)=O)C(=O)[C@@]3(O)C(O)=C1C2=O] |
| S17-3-8 | 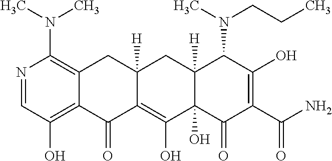 [SMILES: CCCN(C)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-9 | 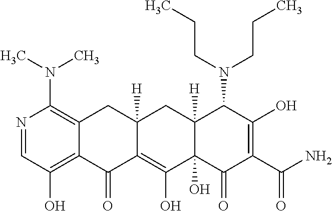 [SMILES: CCCN(CCC)[C@@H]1C(O)=C(C(N)=O)C(=O)[C@@]2(O)C(O)=C3C(=O)c4c(O)cnc(N(C)C)c4C[C@H]3C[C@@H]12] |
| S17-3-10 | 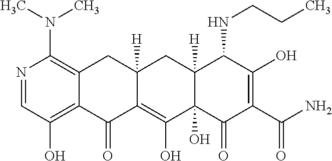 [SMILES: CCCN[C@@H]1C(O)=C(C(N)=O)C(=O)[C@@]2(O)C(O)=C3C(=O)c4c(O)cnc(N(C)C)c4C[C@H]3C[C@@H]12] |
| S17-3-11 | 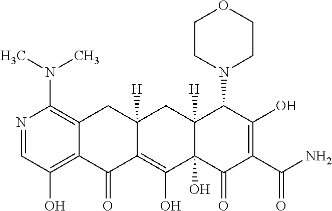 [SMILES: CN(C)c1ncc(O)c2c1C[C@H]1C[C@H]3[C@H](N4CCOCC4)C(O)=C(C(N)=O)C(=O)[C@@]3(O)C(O)=C1C2=O] |
| S18-5-1-1 | 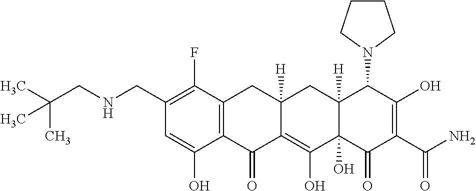 [SMILES: CC(C)(C)CNCc1cc(O)c2c(c1F)C[C@H]1C[C@H]3[C@H](N4CCCC4)C(O)=C(C(N)=O)C(=O)[C@@]3(O)C(O)=C1C2=O] |
| S18-5-1-2 | 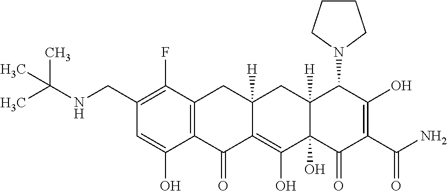 [SMILES: CC(C)(C)NCc1cc(O)c2c(c1F)C[C@H]1C[C@H]3[C@H](N4CCCC4)C(O)=C(C(N)=O)C(=O)[C@@]3(O)C(O)=C1C2=O] |
| S18-5-2-1 | 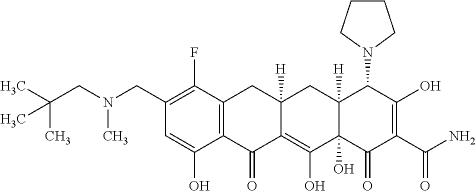 [SMILES: CN(Cc1cc(O)c2c(c1F)C[C@H]1C[C@H]3[C@H](N4CCCC4)C(O)=C(C(N)=O)C(=O)[C@@]3(O)C(O)=C1C2=O)CC(C)(C)C] |
| S18-5-2-2 | 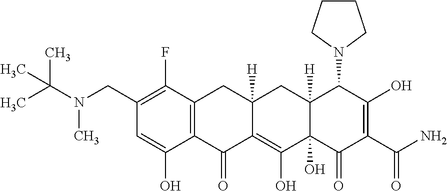 [SMILES: CN(Cc1cc(O)c2c(c1F)C[C@H]1C[C@H]3[C@H](N4CCCC4)C(O)=C(C(N)=O)C(=O)[C@@]3(O)C(O)=C1C2=O)C(C)(C)C] |
| S19-7-1-B (diastereomer B) | 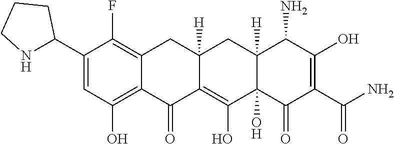 [SMILES: NC(=O)C1=C(O)[C@@H](N)[C@@H]2C[C@@H]3Cc4c(F)c(C5CCCN5)cc(O)c4C(=O)C3=C(O)[C@]2(O)C1=O] |
| S19-7-2 | 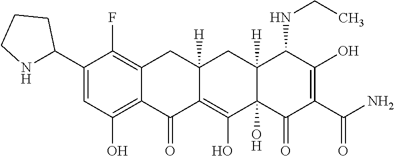 [SMILES: CCN[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-3-A (diastereomer A) S19-7-3-B (diastereomer B) | 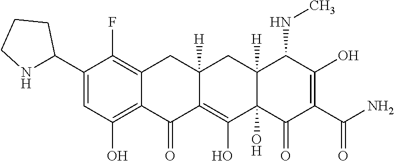 [SMILES: CN[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-4-A (diastereomer A) S19-7-4-B (diastereomer B) | 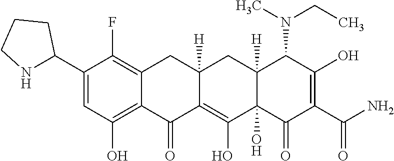 [SMILES: CCN(C)[C@@H]1C(O)=C(C(N)=O)C(=O)[C@@]2(O)C(O)=C3C(=O)c4c(O)cc(C5CCCN5)c(F)c4C[C@H]3C[C@@H]12] |
| S19-7-5-A (diastereomer A) S19-7-5-B (diastereomer B) | 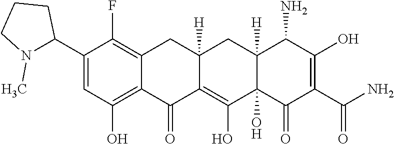 [SMILES: CN1CCCC1c1cc(O)c2c(c1F)C[C@H]1C[C@H]3[C@H](N)C(O)=C(C(N)=O)C(=O)[C@@]3(O)C(O)=C1C2=O] |
| S19-7-6 | 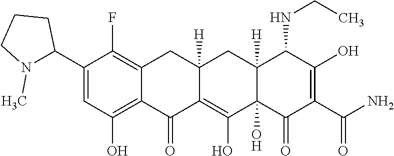 [SMILES: CCN[C@@H]1C(O)=C(C(N)=O)C(=O)[C@@]2(O)C(O)=C3C(=O)c4c(O)cc(C5CCCN5C)c(F)c4C[C@H]3C[C@@H]12] |
| S19-7-7-A (diastereomer A) S19-7-7-B (diastereomer B) | 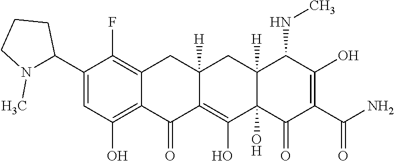 [SMILES: CN[C@@H]1C(O)=C(C(N)=O)C(=O)[C@@]2(O)C(O)=C3C(=O)c4c(O)cc(C5CCCN5C)c(F)c4C[C@H]3C[C@@H]12] |
| S20-4-1 (single diastereomer) | 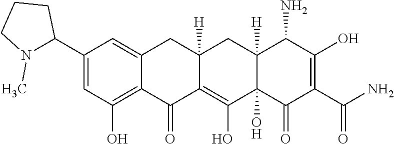 [SMILES: CN1CCCC1c1cc(O)c2c(c1)C[C@H]1C[C@H]3[C@H](N)C(O)=C(C(N)=O)C(=O)[C@@]3(O)C(O)=C1C2=O] |
| S20-4-2 (single diastereomer) | 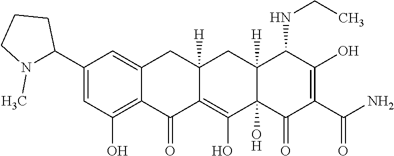 [SMILES: CCN[C@@H]1C(O)=C(C(N)=O)C(=O)[C@@]2(O)C(O)=C3C(=O)c4c(O)cc(C5CCCN5C)cc4C[C@H]3C[C@@H]12] |
| S20-4-3 (single diastereomer) | 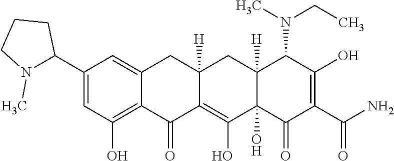 [SMILES: CCN(C)[C@@H]1C(O)=C(C(N)=O)C(=O)[C@@]2(O)C(O)=C3C(=O)c4c(O)cc(C5CCCN5C)cc4C[C@H]3C[C@@H]12] |
| S20-4-4 (single diastereomer) | 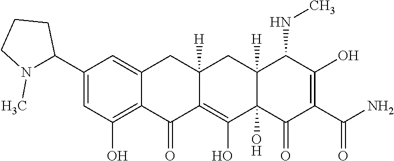 [SMILES: CN[C@@H]1C(O)=C(C(N)=O)C(=O)[C@@]2(O)C(O)=C3C(=O)c4c(O)cc(C5CCCN5C)cc4C[C@H]3C[C@@H]12] |
| S21-5-1 | 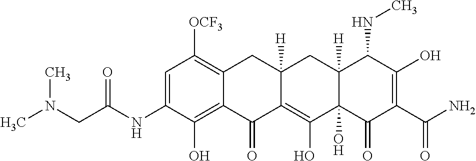 [SMILES: CN[C@@H]1C(O)=C(C(N)=O)C(=O)[C@@]2(O)C(O)=C3C(=O)c4c(O)c(NC(=O)CN(C)C)cc(OC(F)(F)F)c4C[C@H]3C[C@@H]12] |
| S21-5-2 | 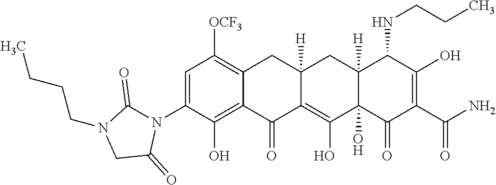 [SMILES: CCCCN1CC(=O)N(c2cc(OC(F)(F)F)c3c(c2O)C(=O)C2=C(O)[C@]4(O)C(=O)C(C(N)=O)=C(O)[C@@H](NCCC)[C@@H]4C[C@@H]2C3)C1=O] |
| S21-5-3 | 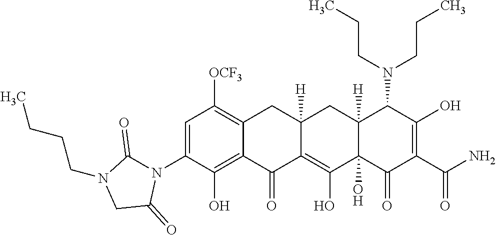 [SMILES: CCCCN1CC(=O)N(c2cc(OC(F)(F)F)c3c(c2O)C(=O)C2=C(O)[C@]4(O)C(=O)C(C(N)=O)=C(O)[C@@H](N(CCC)CCC)[C@@H]4C[C@@H]2C3)C1=O] |
| S21-5-4 | 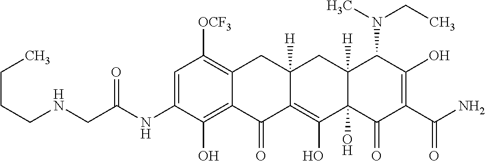 [SMILES: CCCCNCC(=O)Nc1cc(OC(F)(F)F)c2c(c1O)C(=O)C1=C(O)[C@]3(O)C(=O)C(C(N)=O)=C(O)[C@@H](N(C)CC)[C@@H]3C[C@@H]1C2] |

The compound numbers in the tables set forth above reference synthetic schemes in WO2014/03650 all of which are found in U.S. Pat. No. 9,573,895 the entire content of which is hereby incorporated by reference.

In the nineteenth aspect of the 26^{th} embodiment, the compound is represented by any one of the following structural formulas:

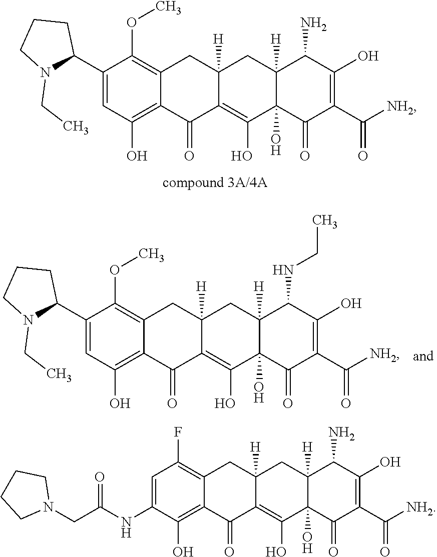

or a pharmaceutically acceptable salt thereof.

In a 27^{th} embodiment, the present invention is a method of treating a hematological cancer comprising administering to a subject in need of treatment an effective amount of a compound represented by any one of structural formulas (X) or (X-1)

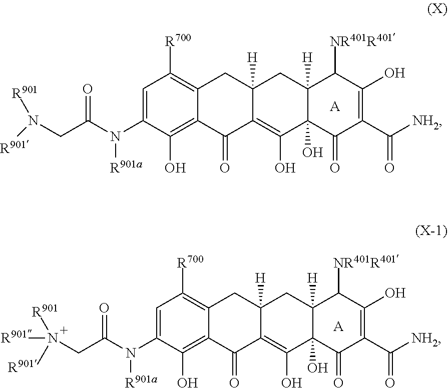

or a pharmaceutically acceptable salt thereof, or a pharmaceutically acceptable composition thereof.

In the first aspect of the 27^{th} embodiment, R⁷⁰⁰, for each occurrence independently, is a halogen; R^{901a}, for each occurrence independently, is H or a C₁-C₄ alkyl; R⁴⁰¹ and R^{401′}, for each occurrence independently, is H or a C₁-C₄ alkyl, a C₁-C₄ hydroxyalkyl, a (C₁₋₄ alkyl)C(O)—, a C₃₋₁₂ carbocyclyl-C(O)—, wherein the carbocyclyl portion is optionally substituted with a hydroxyl group, a (C₁₋₄ alkyl)S(O)₁₋₂—, a (C₁₋₄ alkyl)C(O)NH(C₁₋₄ alkylenyl)-, a (C₁₋₄ alkyl)S(O)₁₋₂NH(C₁₋₄ alkylenyl)-, or a moiety represented by the following structural formula:

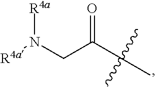

wherein “ [symbol] ” represents the point of attachment to the nitrogen atom, and R^{4a} and R^{4a′}, for each occurrence independently, is H or a C₁-C₄ alkyl, or, taken together with the nitrogen atom to which they are attached, form a 4-13 member heterocyclyl; and R⁹⁰¹, R^{901′}, and R^{901″}, for each occurrence independently, is H, a C₁-C₆ alkyl, a C₁-C₆ haloalkyl, a C₁-C₆ hydroxyalkyl, a (C₁-C₄ alkoxy)-(C₁₋₆)alkyl, an amino-(C₁-C₆) alkyl, a mono- or di-(C₁-C₄ alkyl)amino-(C₁₋₆)alkyl, a C₃₋₁₂ carbocyclyl-(C₀-C₃)alkylenyl, a (4-13 member)heterocyclyl-(C₀-C₃)alkylenyl, or any two of R⁹⁰¹, R^{901′}, and R^{901″}, taken together with the nitrogen atom to which they are attached, form a 4-13 member heterocyclyl.

In the second aspect of the 27^{th} embodiment, R⁷⁰⁰ is F; and R⁹⁰¹, R^{901′}, and R^{901″}, for each occurrence independently, is H, a C₁-C₆ alkyl, a C₁-C₆ haloalkyl, a C₁-C₆ hydroxyalkyl, a (C₁-C₄ alkoxy)-(C₁₋₆)alkyl, an amino-(C₁-C₆) alkyl, a mono- or di-(C₁-C₄ alkyl)amino-(C₁₋₆)alkyl, a C₃₋₁₂ carbocyclyl-(C₀-C₃)alkylenyl, a (4-13 member)heterocyclyl-(C₀-C₃)alkylenyl. The remainder of the values and example values of the variables in structural formulas (X) and (X-1) of the 27^{th} embodiment are as defined above with respect to the first aspect of the 27^{th} embodiment.

In the third aspect of the 27^{th} embodiment, the compound is represented by the structural formula (X); R⁷⁰⁰ is F; and R⁹⁰¹ and R^{901′}, taken together with the nitrogen atom to which they are attached, form a 4-13 member heterocyclyl. The remainder of the values and example values of the variables in structural formulas (X) and (X-1) of the 27^{th} embodiment are as defined above with respect to aspects one through two of the 27^{th} embodiment.

In the fourth aspect of the 27^{th} embodiment, the compound is represented by any one of the following structural formulas, or a pharmaceutically acceptable salt thereof.

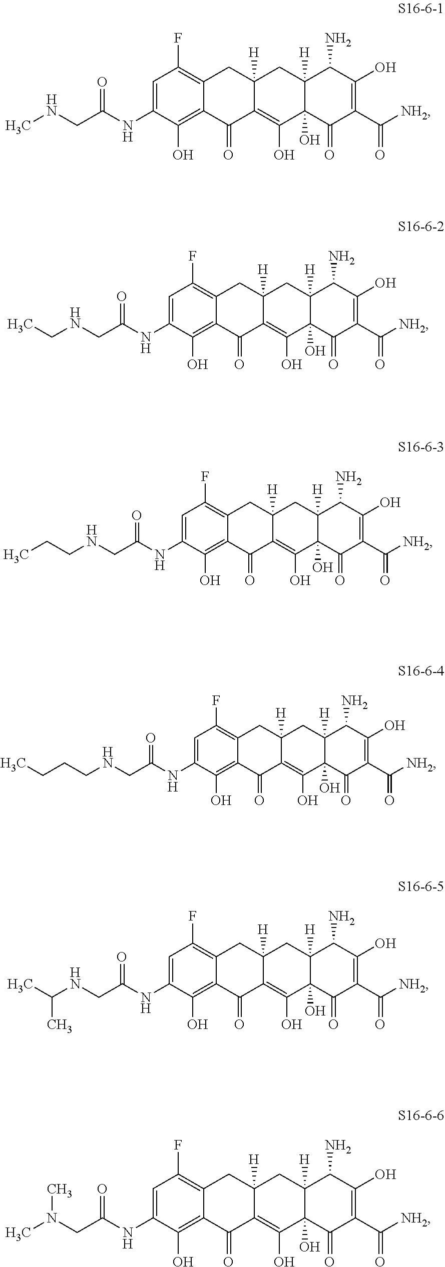

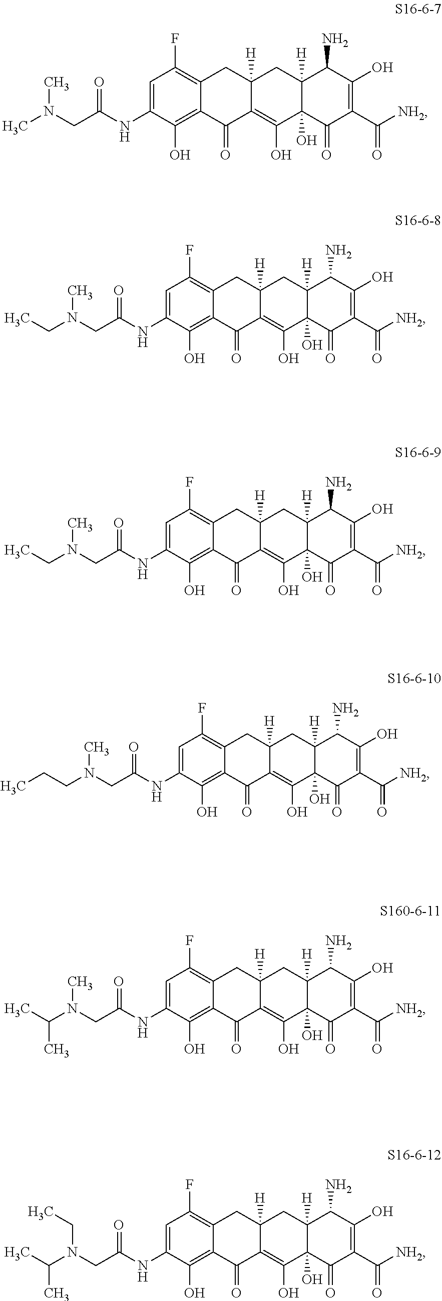

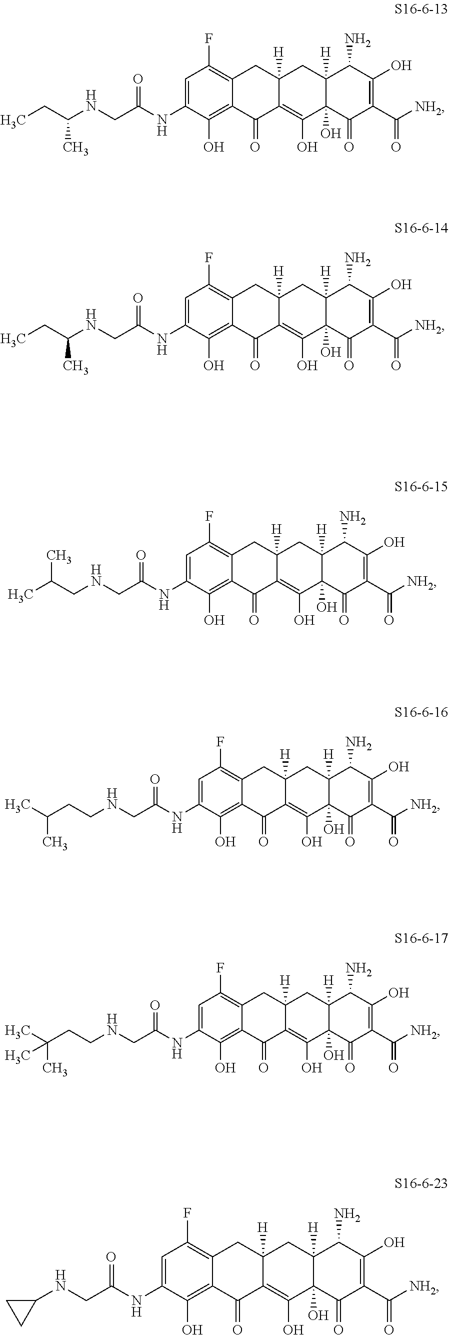

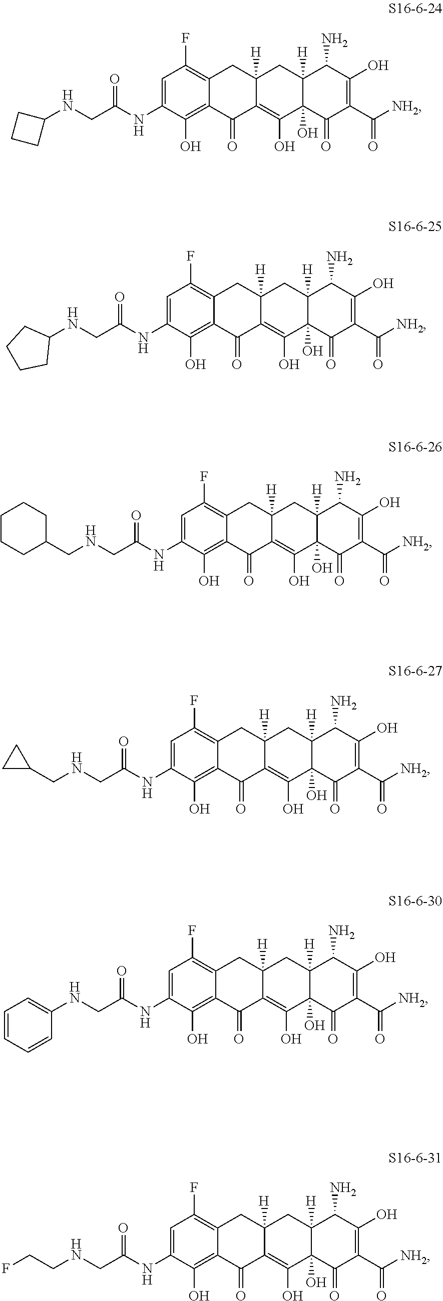

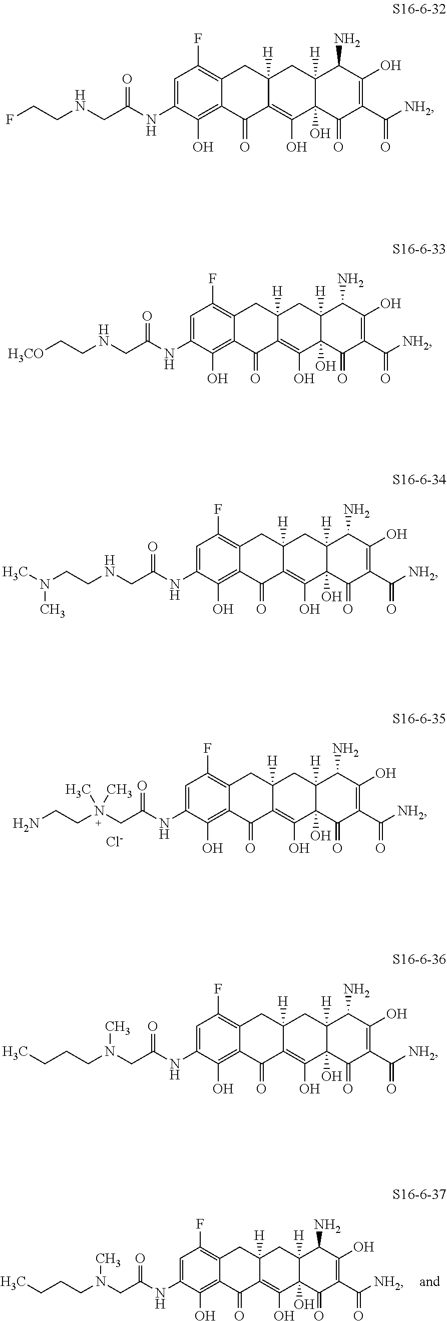

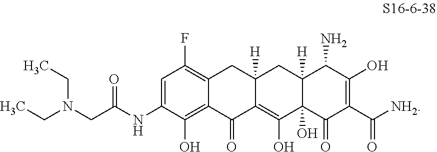 [SMILES: CCN(CC)CC(=O)Nc1cc(F)c2c(c1O)C(=O)C1=C(O)[C@]3(O)C(=O)C(C(N)=O)=C(O)[C@@H](N)[C@@H]3C[C@@H]1C2]

In the fifth aspect of the 27^{th} embodiment, the compound is represented by any one of the following structural formulas, or a pharmaceutically acceptable salt thereof:

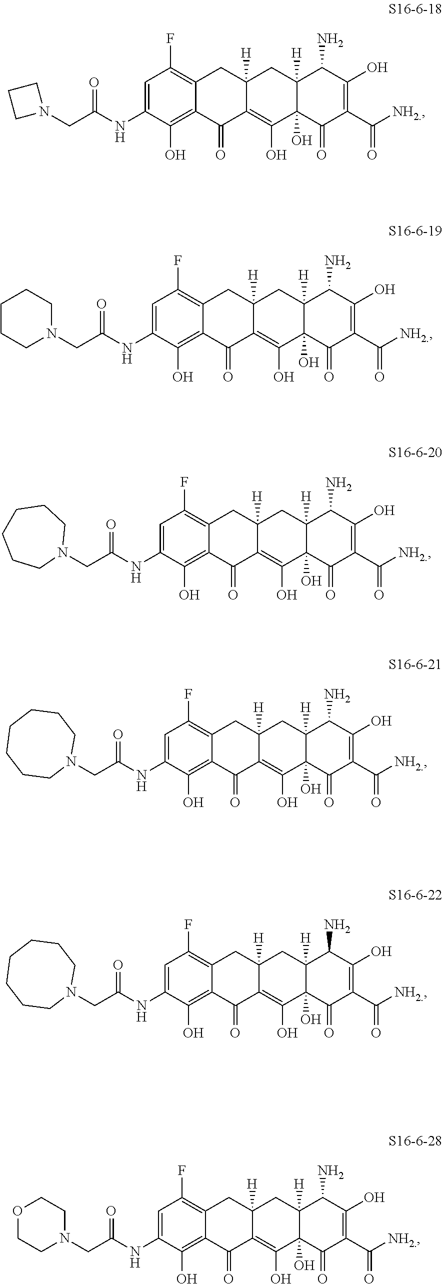

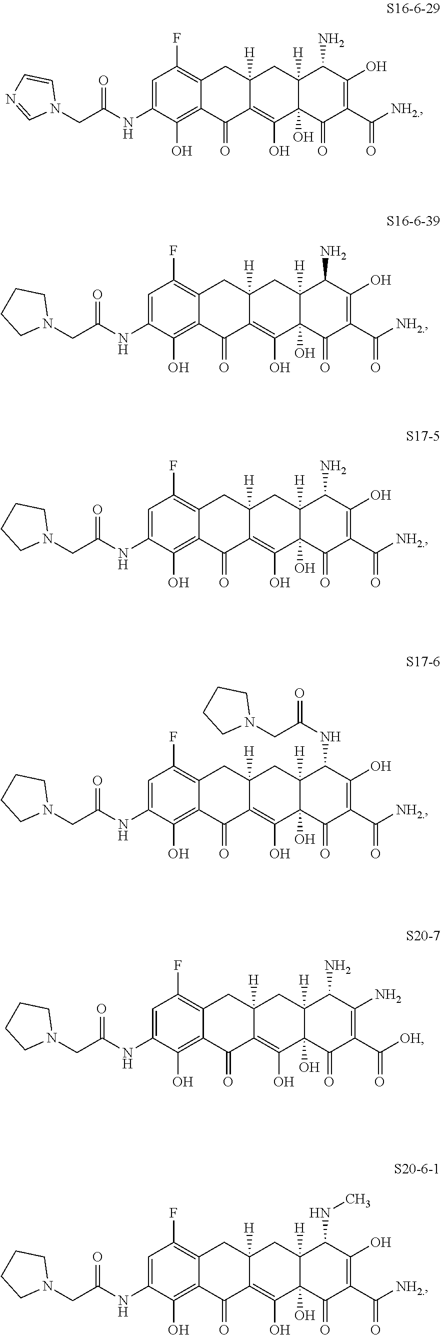

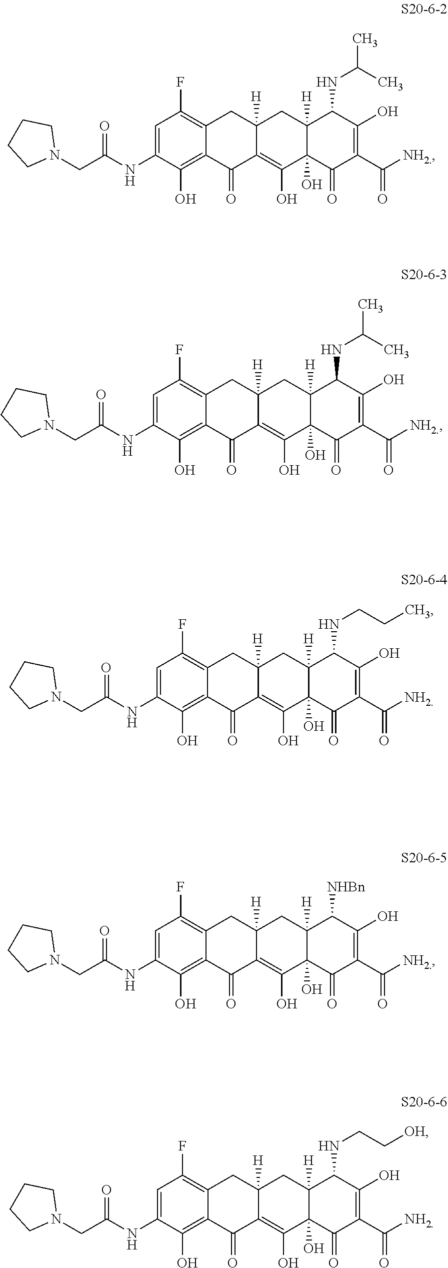

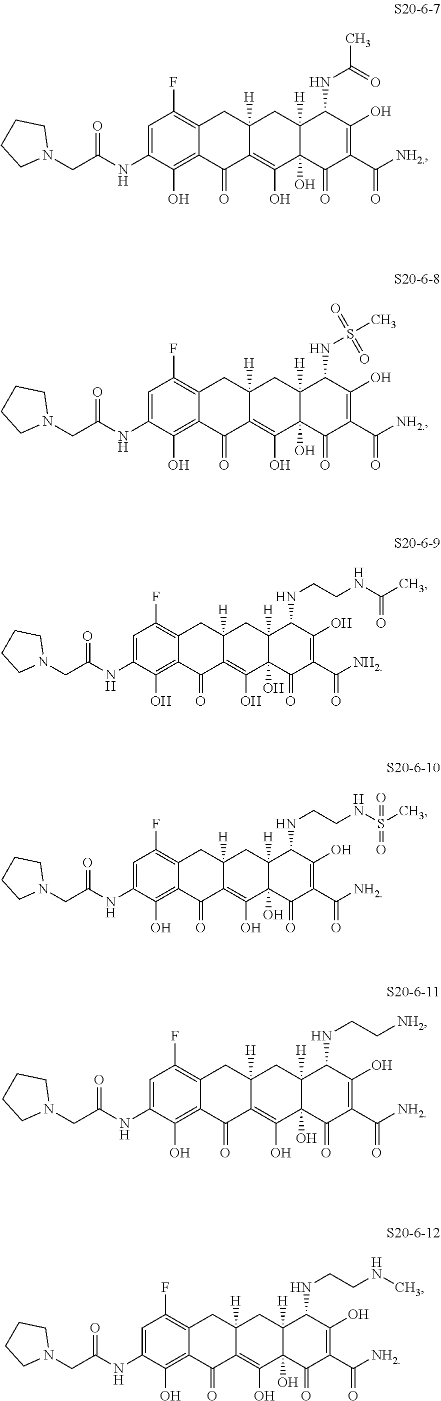

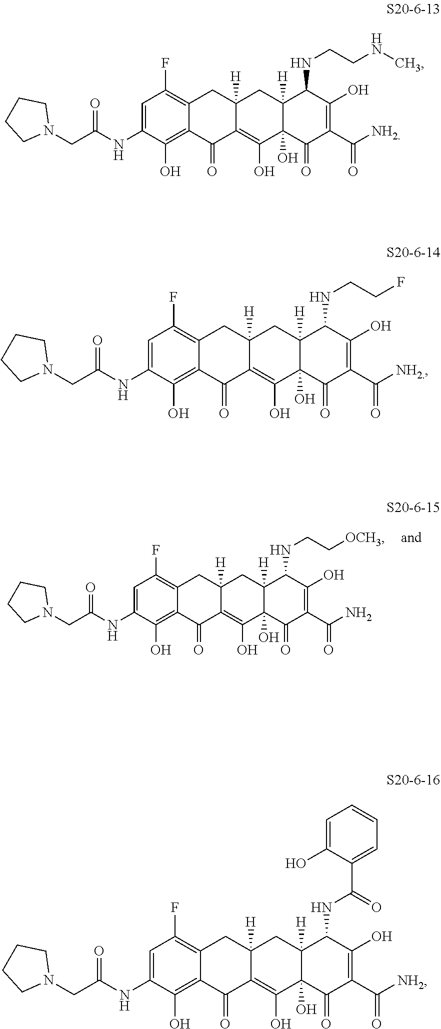 [SMILES: CNCCN[C@H]1C(O)=C(C(N)=O)C(=O)[C@@]2(O)C(O)=C3C(=O)c4c(O)c(NC(=O)CN5CCCC5)cc(F)c4C[C@H]3C[C@@H]12 ; NC(=O)C1=C(O)[C@@H](NCCF)[C@@H]2C[C@@H]3Cc4c(F)cc(NC(=O)CN5CCCC5)c(O)c4C(=O)C3=C(O)[C@]2(O)C1=O ; COCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12 ; "and" ; NC(=O)C1=C(O)[C@@H](NC(=O)c2ccccc2O)[C@@H]2C[C@@H]3Cc4c(F)cc(NC(=O)CN5CCCC5)c(O)c4C(=O)C3=C(O)[C@]2(O)C1=O]

In the 28^{th} embodiment, the present invention is a method of treating a hematological cancer comprising administering to a subject in need of treatment an effective amount of a compound represented by any one of structural formulas (XI), or a pharmaceutically acceptable salt thereof, or a pharmaceutically acceptable composition thereof,

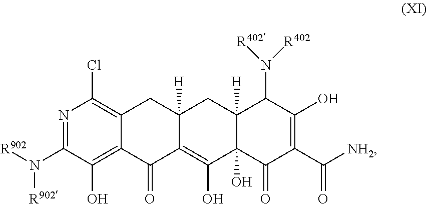

wherein R⁹⁰², R^{902′}, R⁴⁰², and R^{402′}, for each occurrence independently, is H or a C₁-C₆ alkyl. For example, the compound of structural formula (XI) is represented by the following structural formula, or a pharmaceutically acceptable salt thereof:

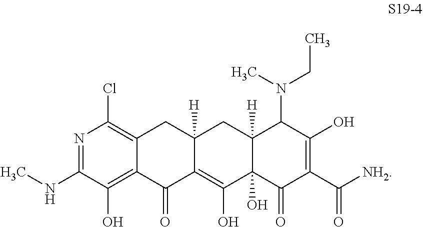 [SMILES: CCN(C)C1C(O)=C(C(N)=O)C(=O)[C@@]2(O)C(O)=C3C(=O)c4c(O)c(NC)nc(Cl)c4C[C@H]3C[C@@H]12]

In the 29^{th} embodiment, the present invention is a compound represented by structural formula (XII), or a pharmaceutically acceptable salt thereof:

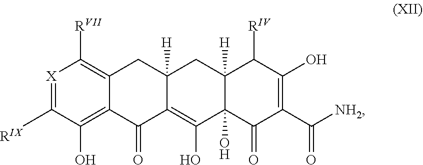

wherein.

| Compound | | | | |
|---|---|---|---|---|
| number | R^{IV} | R^{VII} | R^{IX} | X |
| S16-5 | 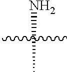 | 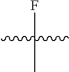 [SMILES: *F] | 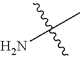 [SMILES: *N] | CH |
| S17-3 | 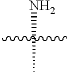 | 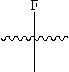 [SMILES: *F] | 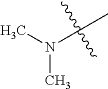 [SMILES: *N(C)C] | CH |
| S16-6-4 | 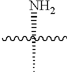 | 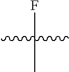 [SMILES: *F] | 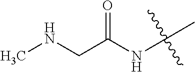 [SMILES: *NC(=O)CNC] | CH |
| S16-6-2 | 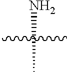 | 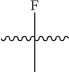 [SMILES: *F] | 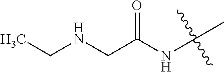 [SMILES: *NC(=O)CNCC] | CH |
| S16-6-31 | 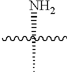 | 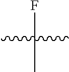 [SMILES: *F] | 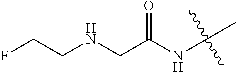 [SMILES: *NC(=O)CNCCF] | CH |
| S16-6-32 | 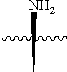 | 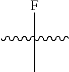 [SMILES: *F] | 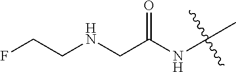 [SMILES: *NC(=O)CNCCF] | CH |
| S16-6-33 | 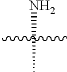 | 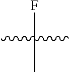 [SMILES: *F] | 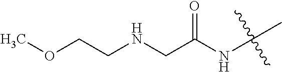 [SMILES: *NC(=O)CNCCOC] | CH |
| S16-6-34 | 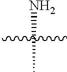 | 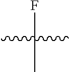 [SMILES: *F] | 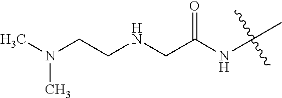 [SMILES: *NC(=O)CNCCN(C)C] | CH |
| S16-6-35 | 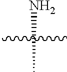 | 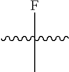 [SMILES: *F] | 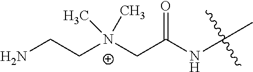 [SMILES: *NC(=O)C[N+](C)(C)CCN] | CH |
| S16-6-3 | 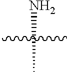 | 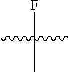 [SMILES: *F] | 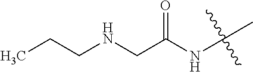 [SMILES: *NC(=O)CNCCC] | CH |
| S16-6-4 | 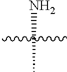 | 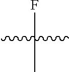 [SMILES: *F] | 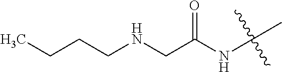 [SMILES: *NC(=O)CNCCCC] | CH |
| S16-6-5 | 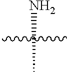 | 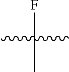 [SMILES: *F] | 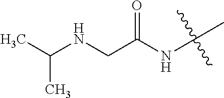 [SMILES: *NC(=O)CNC(C)C] | CH |
| S16-6-15 | 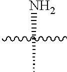 | 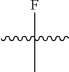 [SMILES: *F] | 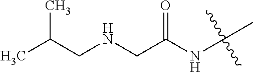 [SMILES: *NC(=O)CNCC(C)C] | CH |
| S16-6-13 | 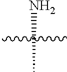 | 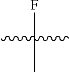 [SMILES: *F] | 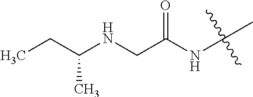 [SMILES: *NC(=O)CN[C@H](C)CC] | CH |
| S16-6-14 | 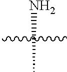 | 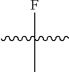 [SMILES: *F] | 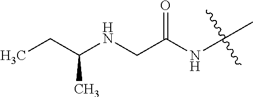 [SMILES: *NC(=O)CN[C@@H](C)CC] | CH |
| S16-6-16 | 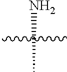 | 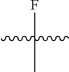 [SMILES: *F] | 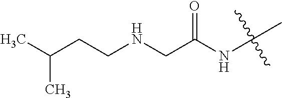 [SMILES: *NC(=O)CNCCC(C)C] | CH |
| S16-6-17 | 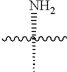 | 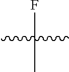 [SMILES: *F] | 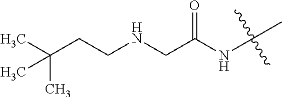 [SMILES: *NC(=O)CNCCC(C)(C)C] | CH |
| S16-6-27 | 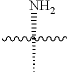 | 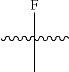 [SMILES: *F] | 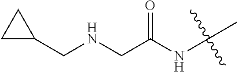 [SMILES: *NC(=O)CNCC1CC1] | CH |
| S16-6-29 | 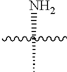 | 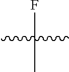 [SMILES: *F] | 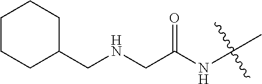 [SMILES: *NC(=O)CNCC1CCCCC1] | CH |
| S16-6-23 | 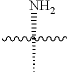 | 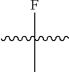 [SMILES: *F] | 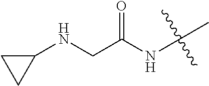 [SMILES: *NC(=O)CNC1CC1] | CH |
| S16-6-24 | 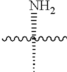 | 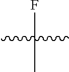 [SMILES: *F] | 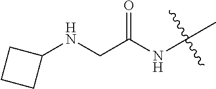 [SMILES: *NC(=O)CNC1CCC1] | CH |
| S16-6-25 | 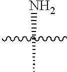 | 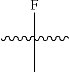 [SMILES: *F] | 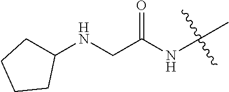 [SMILES: *NC(=O)CNC1CCCC1] | CH |
| S16-6-30 | 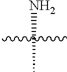 | 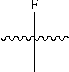 [SMILES: *F] | 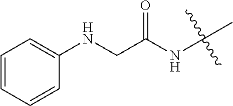 [SMILES: *NC(=O)CNc1ccccc1] | CH |
| S16-6-6 | 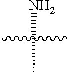 | 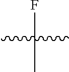 [SMILES: *F] | 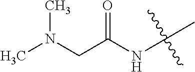 [SMILES: *NC(=O)CN(C)C] | CH |
| S16-6-7 | 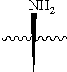 | 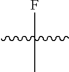 [SMILES: *F] | 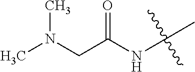 [SMILES: *NC(=O)CN(C)C] | CH |
| S16-6-8 | 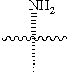 | 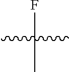 [SMILES: *F] | 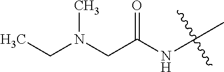 [SMILES: *NC(=O)CN(C)CC] | CH |
| S16-6-9 | 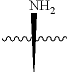 | 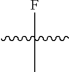 [SMILES: *F] | 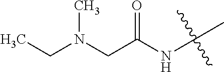 [SMILES: *NC(=O)CN(C)CC] | CH |
| S16-6-10 | 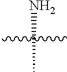 | 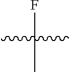 [SMILES: *F] | 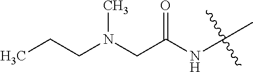 [SMILES: *NC(=O)CN(C)CCC] | CH |
| S16-6-36 | 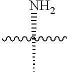 | 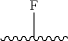 [SMILES: *F] | 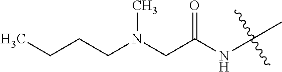 [SMILES: *NC(=O)CN(C)CCCC] | CH |
| S16-6-37 | 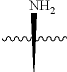 | 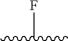 [SMILES: *F] | 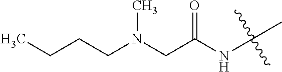 [SMILES: *NC(=O)CN(C)CCCC] | CH |
| S16-6-38 | 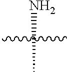 | 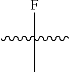 [SMILES: *F] | 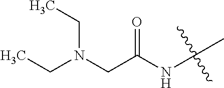 [SMILES: *NC(=O)CN(CC)CC] | CH |
| S16-6-11 | 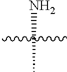 | 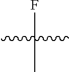 [SMILES: *F] | 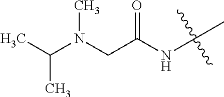 [SMILES: *NC(=O)CN(C)C(C)C] | CH |
| S16-6-12 | 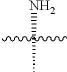 | 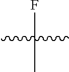 [SMILES: *F] | 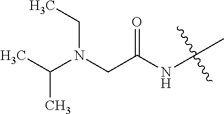 [SMILES: *NC(=O)CN(CC)C(C)C] | CH |
| S16-6-18 | 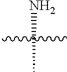 | 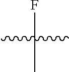 [SMILES: *F] | 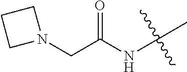 [SMILES: *NC(=O)CN1CCC1] | CH |
| S16-6-39 | 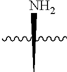 | 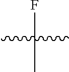 [SMILES: *F] | 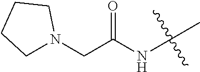 [SMILES: *NC(=O)CN1CCCC1] | CH |
| S17-5 | 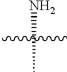 | 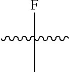 [SMILES: *F] | 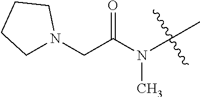 [SMILES: *N(C)C(=O)CN1CCCC1] | CH |
| S17-6 | 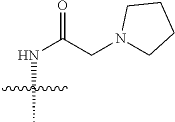 | 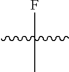 [SMILES: *F] | 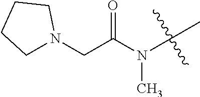 [SMILES: *N(C)C(=O)CN1CCCC1] | CH |
| S16-6-29 | 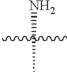 | 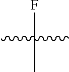 [SMILES: *F] | 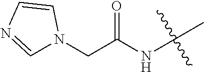 [SMILES: *NC(=O)Cn1ccnc1] | CH |
| S16-6-19 | 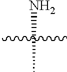 | 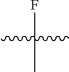 [SMILES: *F] | 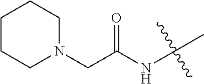 [SMILES: *NC(=O)CN1CCCCC1] | CH |
| S16-6-28 | 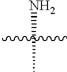 | 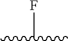 [SMILES: *F] | 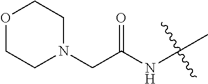 [SMILES: *NC(=O)CN1CCOCC1] | CH |
| S16-6-20 | 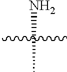 | 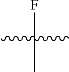 [SMILES: *F] | 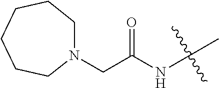 [SMILES: *NC(=O)CN1CCCCCC1] | CH |
| S16-6-21 | 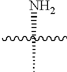 | 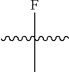 [SMILES: *F] | 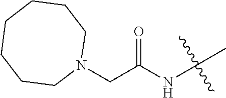 [SMILES: *NC(=O)CN1CCCCCCC1] | CH |
| S16-6-22 | 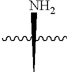 | 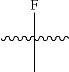 [SMILES: *F] | 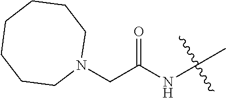 [SMILES: *NC(=O)CN1CCCCCCC1] | CH |
| S20-6-1 | 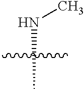 | 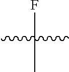 [SMILES: *F] | 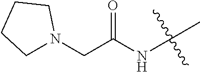 [SMILES: *NC(=O)CN1CCCC1] | CH |
| S20-6-4 | 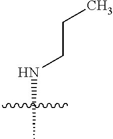 | 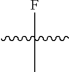 [SMILES: *F] | 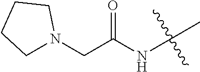 [SMILES: *NC(=O)CN1CCCC1] | CH |
| S20-6-2 | 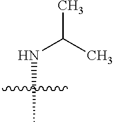 | 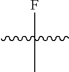 [SMILES: *F] | 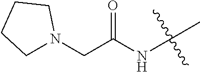 [SMILES: *NC(=O)CN1CCCC1] | CH |
| S20-6-3 | 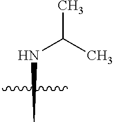 | 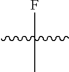 [SMILES: *F] | 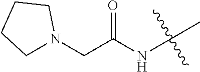 [SMILES: *NC(=O)CN1CCCC1] | CH |
| S20-6-5 | 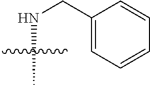 | 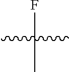 [SMILES: *F] | 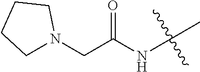 [SMILES: *NC(=O)CN1CCCC1] | CH |
| S20-6-7 | 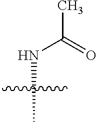 | 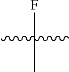 [SMILES: *F] | 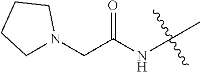 [SMILES: *NC(=O)CN1CCCC1] | CH |
| S20-6-16 | 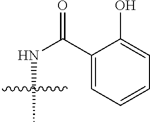 | 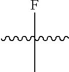 [SMILES: *F] | 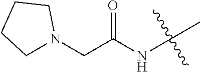 [SMILES: *NC(=O)CN1CCCC1] | CH |
| S20-6-8 | 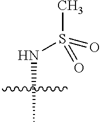 | 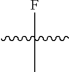 [SMILES: *F] | 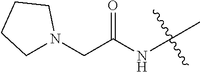 [SMILES: *NC(=O)CN1CCCC1] | CH |
| S20-6-14 | 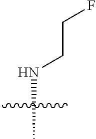 | 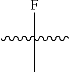 [SMILES: *F] | 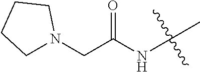 [SMILES: *NC(=O)CN1CCCC1] | CH |
| S20-6-6 | 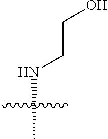 | 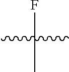 [SMILES: *F] | 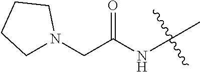 [SMILES: *NC(=O)CN1CCCC1] | CH |
| S20-6-15 | 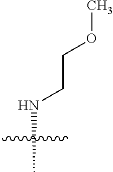 | 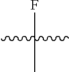 [SMILES: *F] | 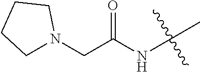 [SMILES: *NC(=O)CN1CCCC1] | CH |
| S20-6-11 | 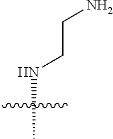 | 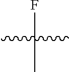 [SMILES: *F] | 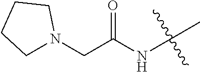 [SMILES: *NC(=O)CN1CCCC1] | CH |
| S20-6-12 | 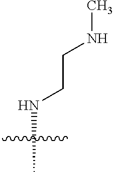 | 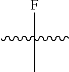 [SMILES: *F] | 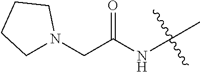 [SMILES: *NC(=O)CN1CCCC1] | CH |
| S20-6-13 | 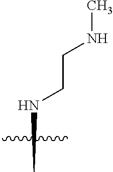 | 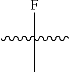 [SMILES: *F] | 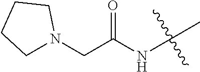 [SMILES: *NC(=O)CN1CCCC1] | CH |
| S20-6-9 | 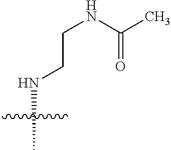 | 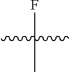 [SMILES: *F] | 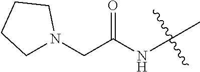 [SMILES: *NC(=O)CN1CCCC1] | CH |
| S20-6-10 | 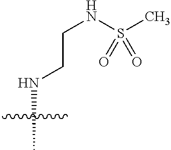 | 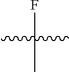 [SMILES: *F] | 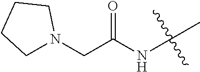 [SMILES: *NC(=O)CN1CCCC1] | CH |
| S19-4 | 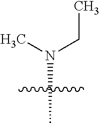 | 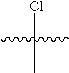 [SMILES: *Cl] | 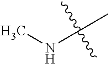 [SMILES: *NC] | N |

In the 30^{th} embodiment, the present invention is a method of treating a hematological cancer comprising administering to a subject in need of treatment an effective amount of a compound represented by the following structural formula, or a pharmaceutically acceptable salt thereof, or a pharmaceutically acceptable composition thereof:

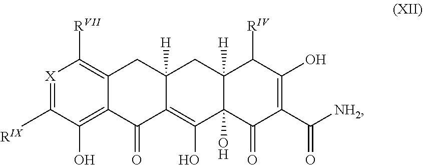

wherein:

| Compound | | | | |
|---|---|---|---|---|
| number | R^{IV} | R^{VII} | R^{IX} | X |
| K43 | 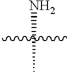 | 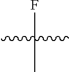 [SMILES: *F] | 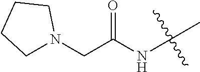 [SMILES: *NC(=O)CN1CCCC1] | CH |
| K44 | 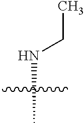 | 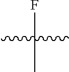 [SMILES: *F] | 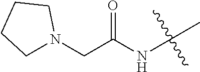 [SMILES: *NC(=O)CN1CCCC1] | CH |
| K45 | 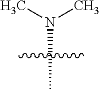 | 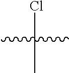 [SMILES: *Cl] | 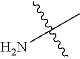 [SMILES: *N] | N |
| K46 | 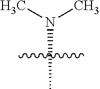 | 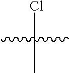 [SMILES: *Cl] | 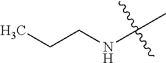 [SMILES: *NCCC] | N |
| K47 | 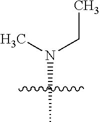 | 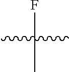 [SMILES: *F] | 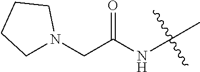 [SMILES: *NC(=O)CN1CCCC1] | CH |
| K48 | 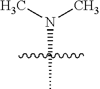 | 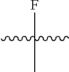 [SMILES: *F] | 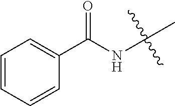 [SMILES: *NC(=O)c1ccccc1] | CH |
| K49 | 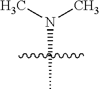 | 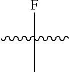 [SMILES: *F] | 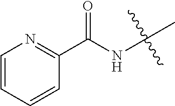 [SMILES: *NC(=O)c1ccccn1] | CH |
| K50 | 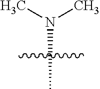 | 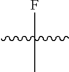 [SMILES: *F] | 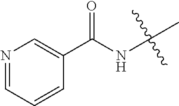 [SMILES: *NC(=O)c1cccnc1] | CH |
| K51 | 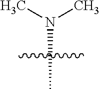 | 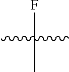 [SMILES: *F] | 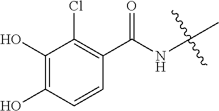 [SMILES: *NC(=O)c1ccc(O)c(O)c1Cl] | CH |
| K52 | 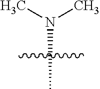 | 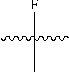 [SMILES: *F] | 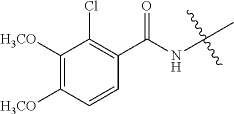 [SMILES: *NC(=O)c1ccc(OC)c(OC)c1Cl] | CH |
| K53 | 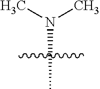 | 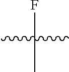 [SMILES: *F] | 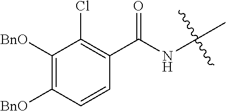 [SMILES: *NC(=O)c1ccc(OCc2ccccc2)c(OCc2ccccc2)c1Cl] | CH |
| K54 | 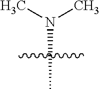 | 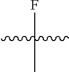 [SMILES: *F] | 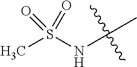 [SMILES: *NS(C)(=O)=O] | CH |
| K55 | 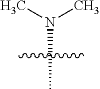 | 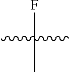 [SMILES: *F] | 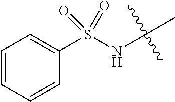 [SMILES: *NS(=O)(=O)c1ccccc1] | CH |
| K56 | 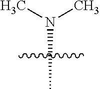 | 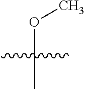 [SMILES: *OC] | 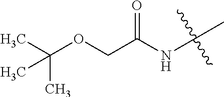 [SMILES: *NC(=O)COC(C)(C)C] | CH |
| K57 | 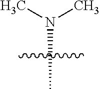 | 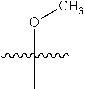 [SMILES: *OC] | 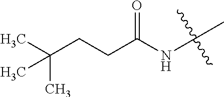 [SMILES: *NC(=O)CCC(C)(C)C] | CH |
| K58 | 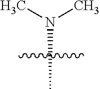 | 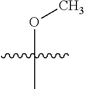 [SMILES: *OC] | 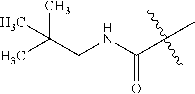 [SMILES: *C(=O)NCC(C)(C)C] | CH |
| K59 | 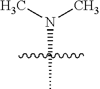 | 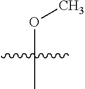 [SMILES: *OC] | 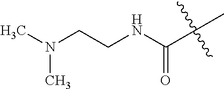 [SMILES: *C(=O)NCCN(C)C] | CH |
| K60 | 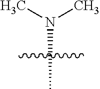 | 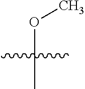 [SMILES: *OC] | 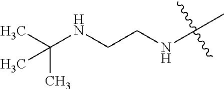 [SMILES: *NCCNC(C)(C)C] | CH |
| K61 | 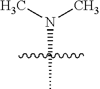 | 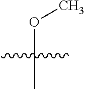 [SMILES: *OC] | 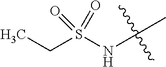 [SMILES: *NS(=O)(=O)CC] | CH |
| K62 | 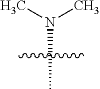 | 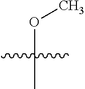 [SMILES: *OC] | 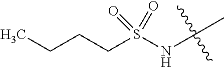 [SMILES: *NS(=O)(=O)CCCC] | CH |
| K63 | 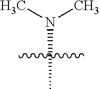 | 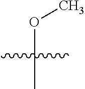 [SMILES: *OC] | 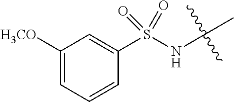 [SMILES: *NS(=O)(=O)c1cccc(OC)c1] | CH |
| K64 | 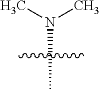 | 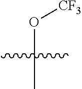 [SMILES: *OC(F)(F)F] | 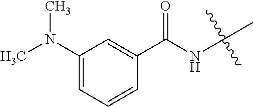 [SMILES: *NC(=O)c1cccc(N(C)C)c1] | CH |
| K65 | 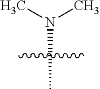 | 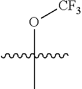 [SMILES: *OC(F)(F)F] | 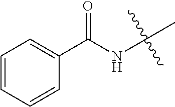 [SMILES: *NC(=O)c1ccccc1] | CH |

In the 31^{st} embodiment, the present invention is a method of treating a hematological cancer comprising administering to a subject in need of treatment an effective amount of a compound represented by the following structural formula

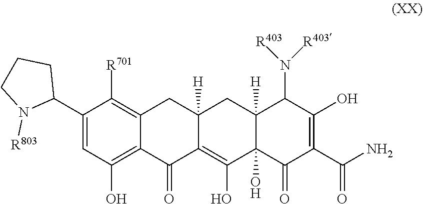

or a pharmaceutically acceptable salt thereof, or a pharmaceutically acceptable composition thereof.

In the first aspect of the 31^{st} embodiment, R⁸⁰³ is H, a C₁₋₆ alkyl, a C₁₋₆ haloalkyl, C₁₋₆ hydroxyalkyl, a C₃₋₁₂ carbocyclyl-(C₀₋₃)alkylenyl, an amino-(C₁-C₄) alkyl, a mono- or di-(C₁-C₄ alkyl)amino-(C₁₋₄)alkyl, a (4-13 member)heterocyclyl-(C₀-C₃)alkylenyl, wherein the heterocyclyl portion is optionally substituted with a C₁₋₃ alkyl; R⁷⁰¹ is H, a C₁₋₄ alkyloxy, —OH, C₁₋₄ alkyl, a C₁₋₄ haloalkyl, C₁₋₄ hydroxyalkyl, C₁₋₄ haloalkoxy; and R⁴⁰³ and R^{403′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH(C₁₋₄ alkylenyl)-; a (C₁₋₄ alkyl)S(O)₁₋₂NH(C₁₋₄ alkylenyl)-; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 31^{st} embodiment, R⁷⁰¹ is —OCH₃, and R⁸⁰³ is ethyl. The remainder of the values and example values of the variables in structural formula (XX) of the 31^{st} embodiment are as defined above with respect to the first aspect of the 31^{st} embodiment.

In the third aspect of the 31^{st} embodiment, R⁷⁰¹ is —OCH₃, and R⁴⁰³ and R^{403′} each is hydrogen. The remainder of the values and example values of the variables in structural formula (XX) of the 31^{st} embodiment are as defined above with respect to the first or second aspects of the 31^{st} embodiment.

In the fourth aspect of the 31^{st} embodiment, R⁸⁰³ is ethyl and R⁴⁰³ and R^{403′} each is hydrogen. The remainder of the values and example values of the variables in structural formula (XX) of the 31^{st} embodiment are as defined above with respect to aspects one through three of the 31^{st} embodiment.

In the fifth aspect of the 31^{st} embodiment, R⁷⁰¹ is a —OCF₃, and R⁸⁰³ is methyl. The remainder of the values and example values of the variables in structural formula (XX) of the 31^{st} embodiment are as defined above with respect to aspects one through four of the 31^{st} embodiment.

In the sixth aspect of the 31^{st} embodiment, the compound is represented by any one of the following structural formulas, or a pharmaceutically acceptable salt thereof:

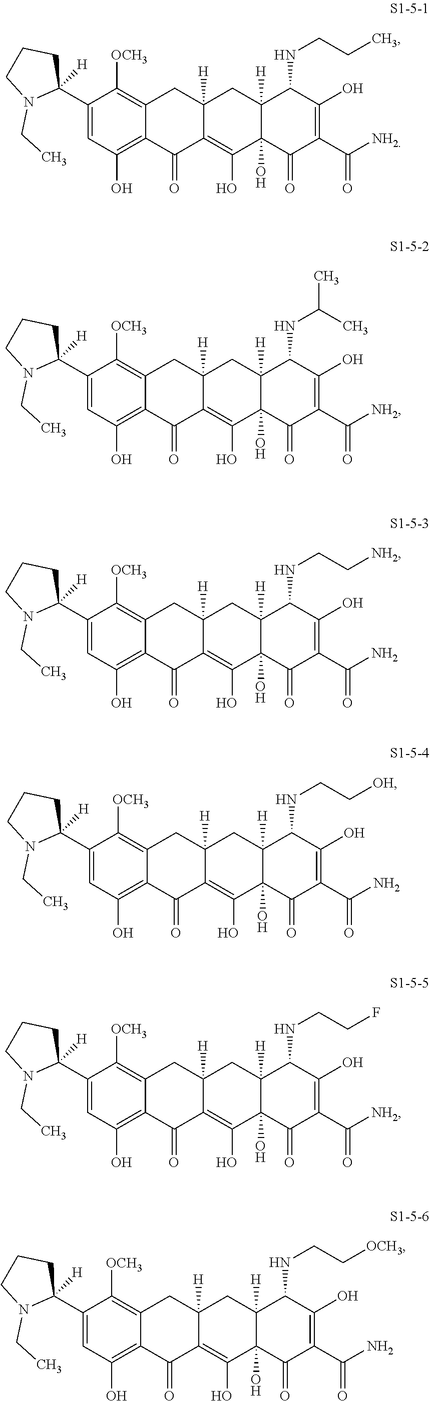

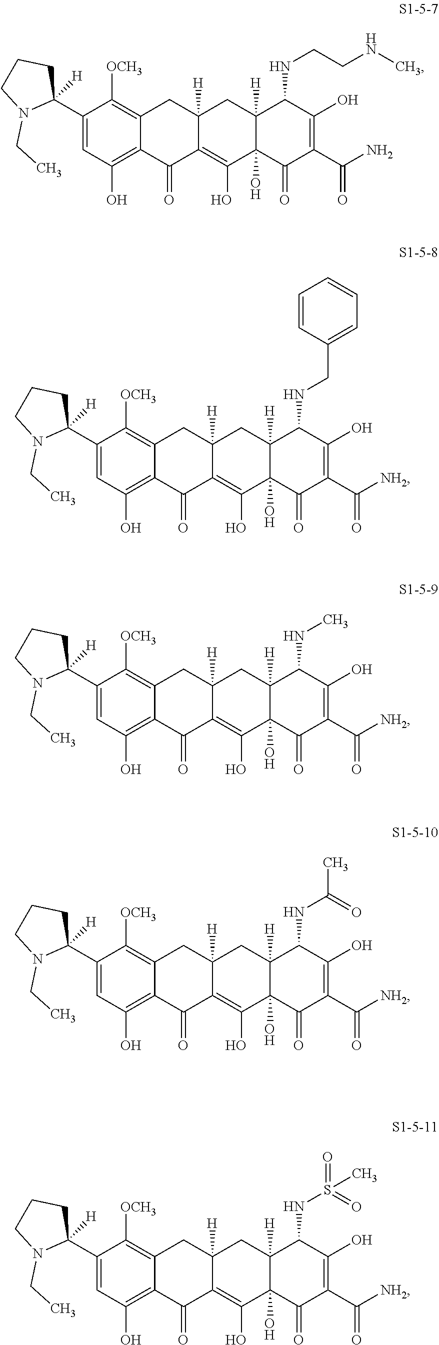 [SMILES: CCN1CCC[C@H]1c1cc(O)c2c(c1OC)C[C@H]1C[C@H]3[C@H](NCCNC)C(O)=C(C(N)=O)C(=O)[C@@]3(O)C(O)=C1C2=O ; CCN1CCC[C@H]1c1cc(O)c2c(c1OC)C[C@H]1C[C@H]3[C@H](NCc4ccccc4)C(O)=C(C(N)=O)C(=O)[C@@]3(O)C(O)=C1C2=O ; CCN1CCC[C@H]1c1cc(O)c2c(c1OC)C[C@H]1C[C@H]3[C@H](NC)C(O)=C(C(N)=O)C(=O)[C@@]3(O)C(O)=C1C2=O ; CCN1CCC[C@H]1c1cc(O)c2c(c1OC)C[C@H]1C[C@H]3[C@H](NC(C)=O)C(O)=C(C(N)=O)C(=O)[C@@]3(O)C(O)=C1C2=O ; CCN1CCC[C@H]1c1cc(O)c2c(c1OC)C[C@H]1C[C@H]3[C@H](NS(C)(=O)=O)C(O)=C(C(N)=O)C(=O)[C@@]3(O)C(O)=C1C2=O]

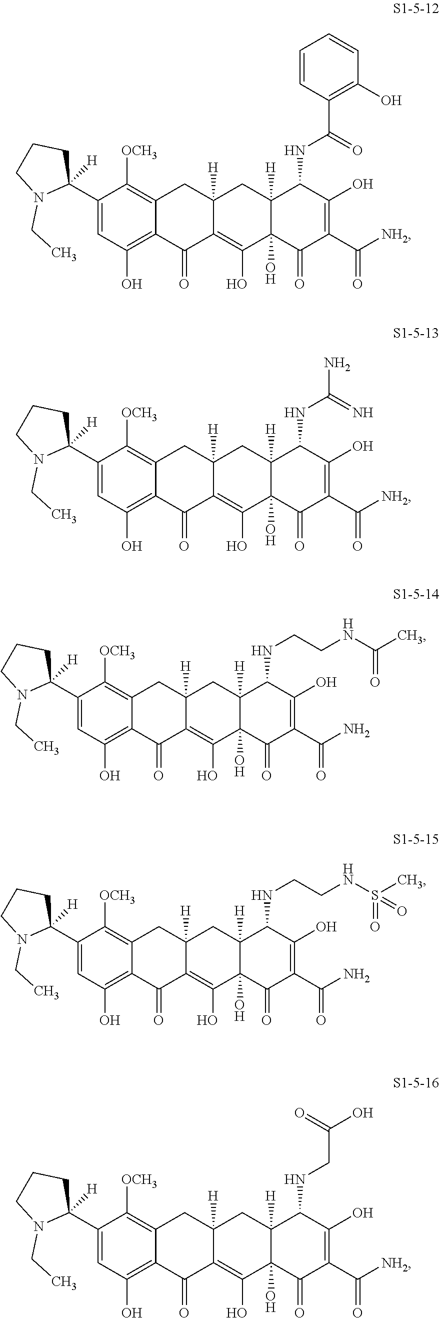

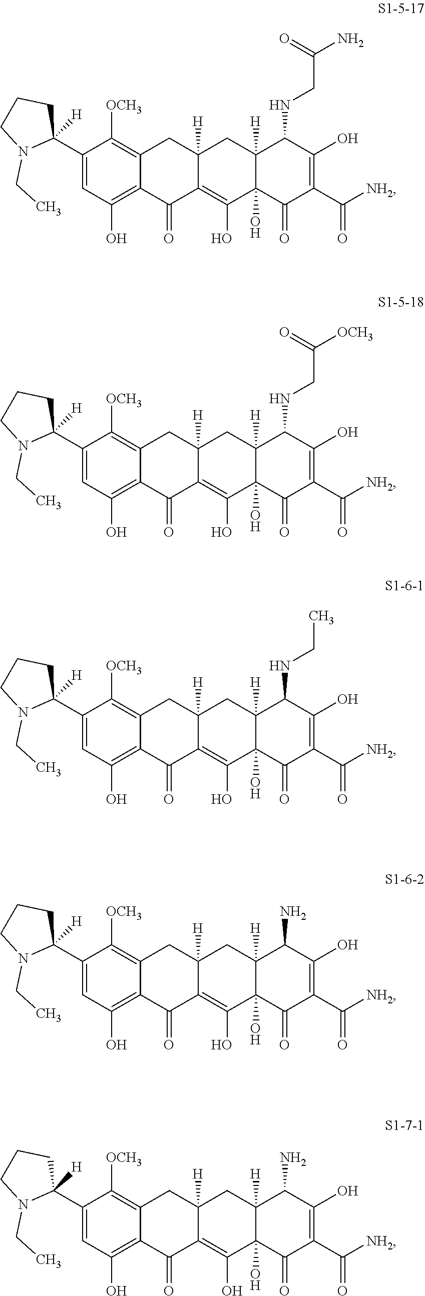 [SMILES: CCN1CCC[C@H]1c1cc(O)c2c(c1OC)C[C@H]1C[C@H]3[C@H](NCC(N)=O)C(O)=C(C(N)=O)C(=O)[C@@]3(O)C(O)=C1C2=O ; CCN1CCC[C@H]1c1cc(O)c2c(c1OC)C[C@H]1C[C@H]3[C@H](NCC(=O)OC)C(O)=C(C(N)=O)C(=O)[C@@]3(O)C(O)=C1C2=O ; CCN[C@H]1C(O)=C(C(N)=O)C(=O)[C@@]2(O)C(O)=C3C(=O)c4c(O)cc([C@@H]5CCCN5CC)c(OC)c4C[C@H]3C[C@@H]12 ; CCN1CCC[C@H]1c1cc(O)c2c(c1OC)C[C@H]1C[C@H]3[C@@H](N)C(O)=C(C(N)=O)C(=O)[C@@]3(O)C(O)=C1C2=O ; CCN1CCC[C@@H]1c1cc(O)c2c(c1OC)C[C@H]1C[C@H]3[C@H](N)C(O)=C(C(N)=O)C(=O)[C@@]3(O)C(O)=C1C2=O]

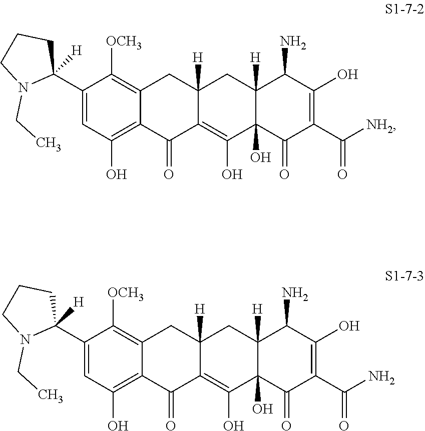 [SMILES: CCN1CCC[C@H]1c1cc(O)c2c(c1OC)C[C@@H]1C[C@@H]3[C@@H](N)C(O)=C(C(N)=O)C(=O)[C@]3(O)C(O)=C1C2=O ; CCN1CCC[C@@H]1c1cc(O)c2c(c1OC)C[C@@H]1C[C@@H]3[C@@H](N)C(O)=C(C(N)=O)C(=O)[C@]3(O)C(O)=C1C2=O]

In the seventh aspect of the 31^{st} embodiment, the compound is represented by any one of the following structural formulas, or a pharmaceutically acceptable salt thereof:

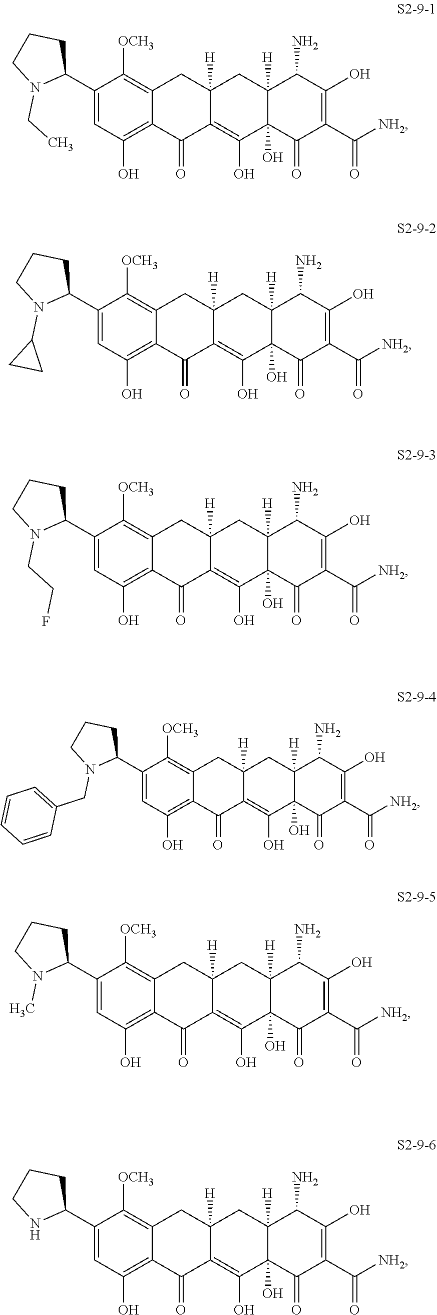

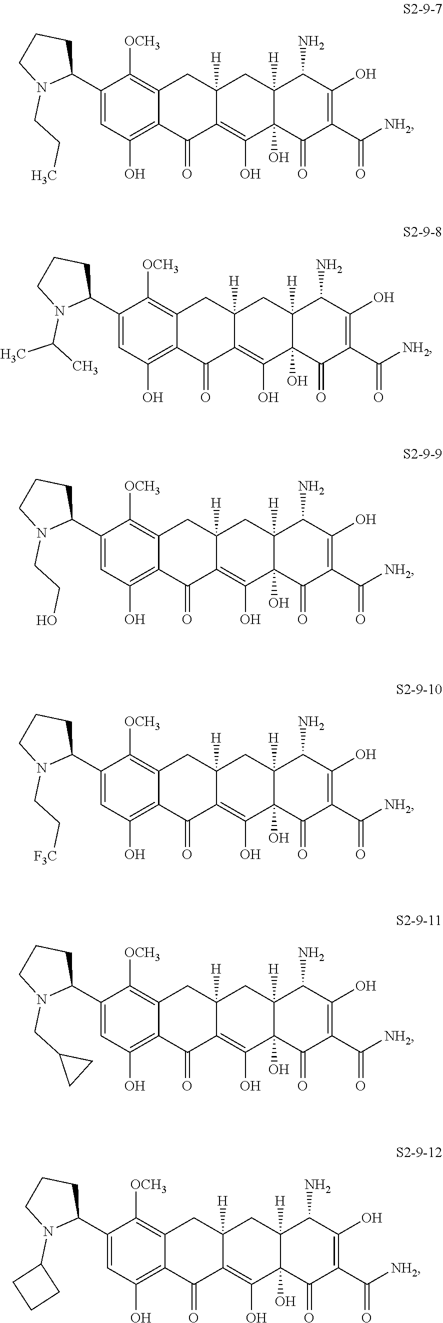

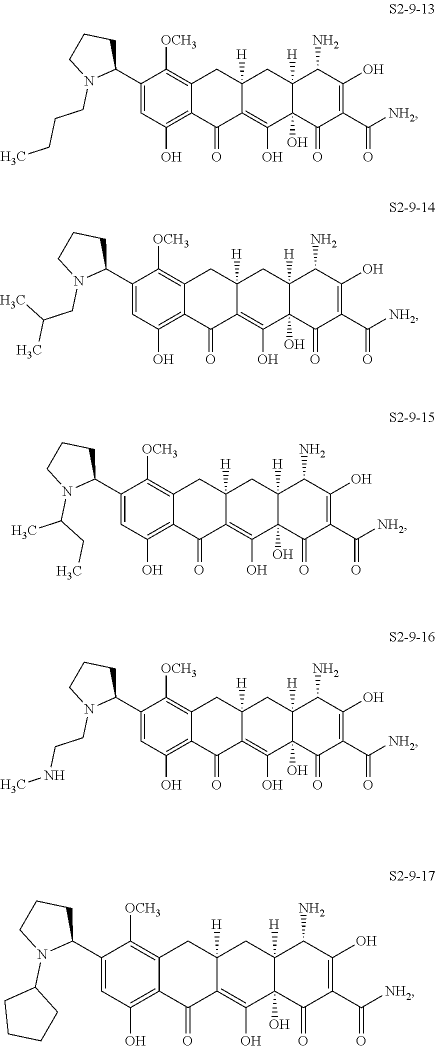 [SMILES: CCCCN1CCC[C@H]1c1cc(O)c2c(c1OC)C[C@H]1C[C@H]3[C@H](N)C(O)=C(C(N)=O)C(=O)[C@@]3(O)C(O)=C1C2=O ; COc1c([C@@H]2CCCN2CC(C)C)cc(O)c2c1C[C@H]1C[C@H]3[C@H](N)C(O)=C(C(N)=O)C(=O)[C@@]3(O)C(O)=C1C2=O ; CCC(C)N1CCC[C@H]1c1cc(O)c2c(c1OC)C[C@H]1C[C@H]3[C@H](N)C(O)=C(C(N)=O)C(=O)[C@@]3(O)C(O)=C1C2=O ; CNCCN1CCC[C@H]1c1cc(O)c2c(c1OC)C[C@H]1C[C@H]3[C@H](N)C(O)=C(C(N)=O)C(=O)[C@@]3(O)C(O)=C1C2=O ; COc1c([C@@H]2CCCN2C2CCCC2)cc(O)c2c1C[C@H]1C[C@H]3[C@H](N)C(O)=C(C(N)=O)C(=O)[C@@]3(O)C(O)=C1C2=O]

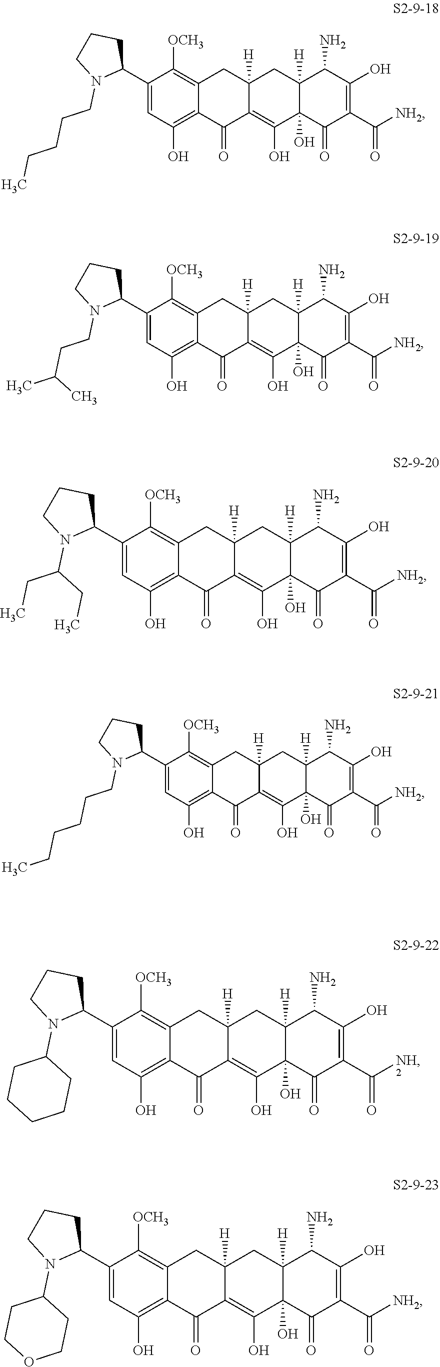

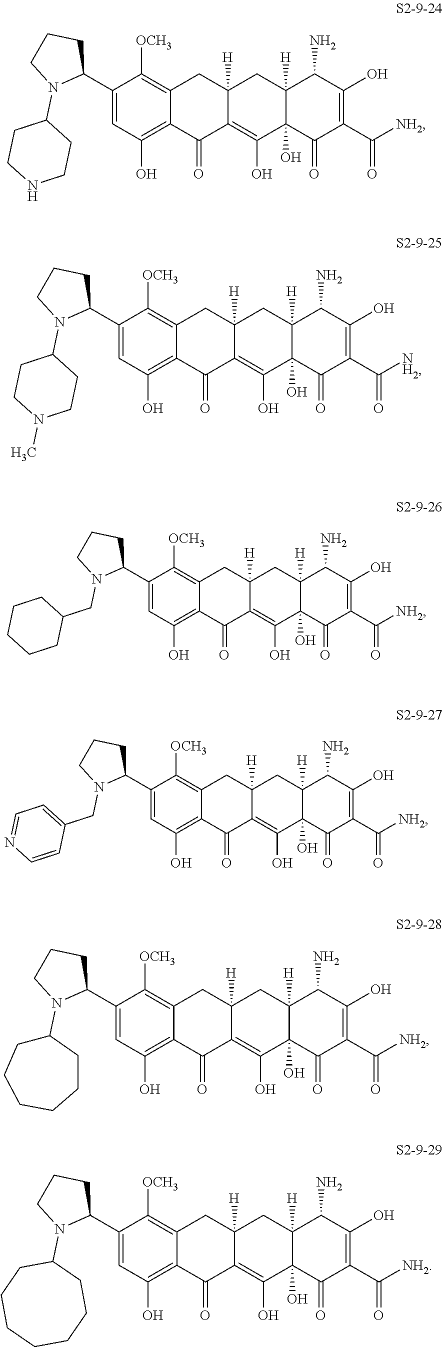

In the eighth aspect of the 31^{st} embodiment, the compound is represented by any one of the following structural formulas, or a pharmaceutically acceptable salt thereof:

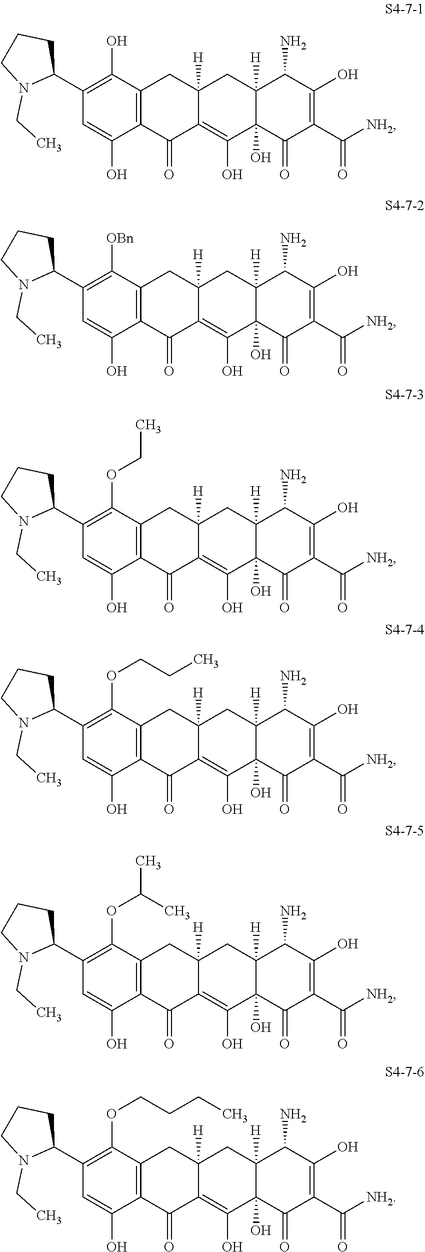

In the ninth aspect of the 31^{st} embodiment, the compound is represented by any one of the following structural formulas, or a pharmaceutically acceptable salt thereof:

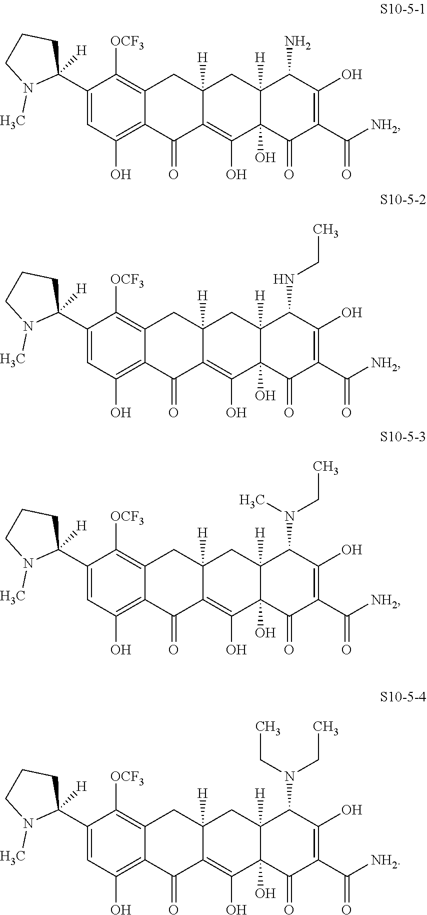 [SMILES: CN1CCC[C@H]1c1cc(O)c2c(c1OC(F)(F)F)C[C@H]1C[C@H]3[C@H](N)C(O)=C(C(N)=O)C(=O)[C@@]3(O)C(O)=C1C2=O ; CCN[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12 ; CCN(C)[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12]

In the tenth aspect of the 31^{st} embodiment, the compound is represented by any one of the following structural formulas, or a pharmaceutically acceptable salt thereof:

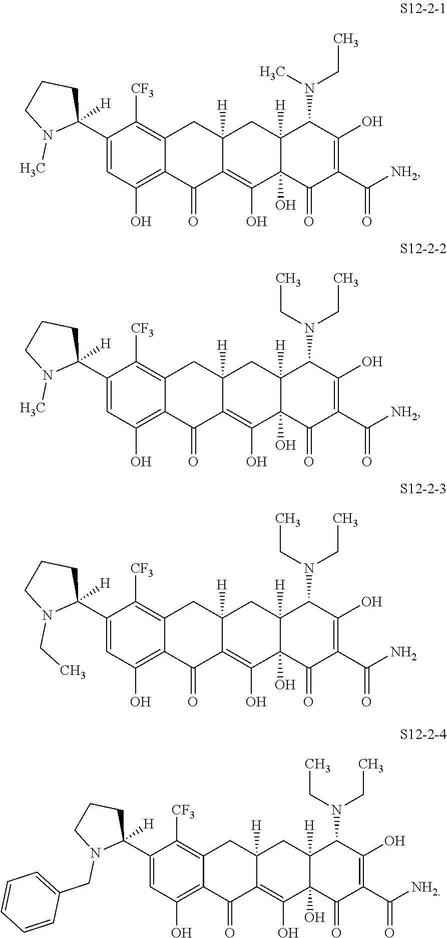 [SMILES: CCN(C)[C@@H]1C(O)=C(C(N)=O)C(=O)[C@@]2(O)C(O)=C3C(=O)c4c(O)cc([C@@H]5CCCN5C)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc([C@@H]5CCCN5C)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc([C@@H]5CCCN5CC)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc([C@@H]5CCCN5Cc5ccccc5)c(C(F)(F)F)c4C[C@H]3C[C@@H]12]

In the 32^{nd} embodiment, the present invention is method of treating a hematological cancer comprising administering to a subject in need of treatment an effective amount of a compound represented by the following structural formula:

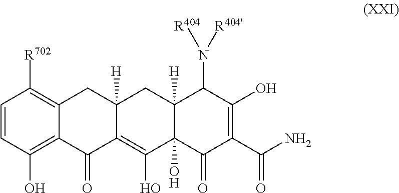

or a pharmaceutically acceptable salt thereof, or a pharmaceutically acceptable composition thereof. In a first aspect of the 32nf embodiment, R⁷⁰² is H, a halogen, a C₁₋₄ alkyloxy, —OH, C₁₋₄ alkyl, a C₁₋₄ haloalkyl, C₁₋₄ hydroxyalkyl, C₁₋₄ haloalkoxy; and R⁴⁰⁴ and R^{404′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH—C₁₋₄ alkylenyl; a (C₁₋₄ alkyl)S(O)₁₋₂NH—C₁₋₄ alkylenyl; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 32^{st} embodiment, R⁷⁰² is a C₁₋₄ haloalkyl. The remainder of the values and example values of the variables in structural formula (XXI) of the 32^{st} embodiment are as defined above with respect to aspect one of the 32^{st} embodiment.

In the third aspect of the 32^{st} embodiment, R⁷⁰² is H or a halogen. The remainder of the values and example values of the variables in structural formula (XXI) of the 32^{st} embodiment are as defined above with respect to aspects one or two of the 32^{st} embodiment.

In the fourth aspect of the 32^{st} embodiment, R⁷⁰² is —OCH₃. The remainder of the values and example values of the variables in structural formula (XXI) of the 32^{st} embodiment are as defined above with respect to aspect o to three of the 32^{st} embodiment.

In the fifth aspect of the 32^{st} embodiment, the compound is represented by any one of the following structural formulas, or a pharmaceutically acceptable salt thereof:

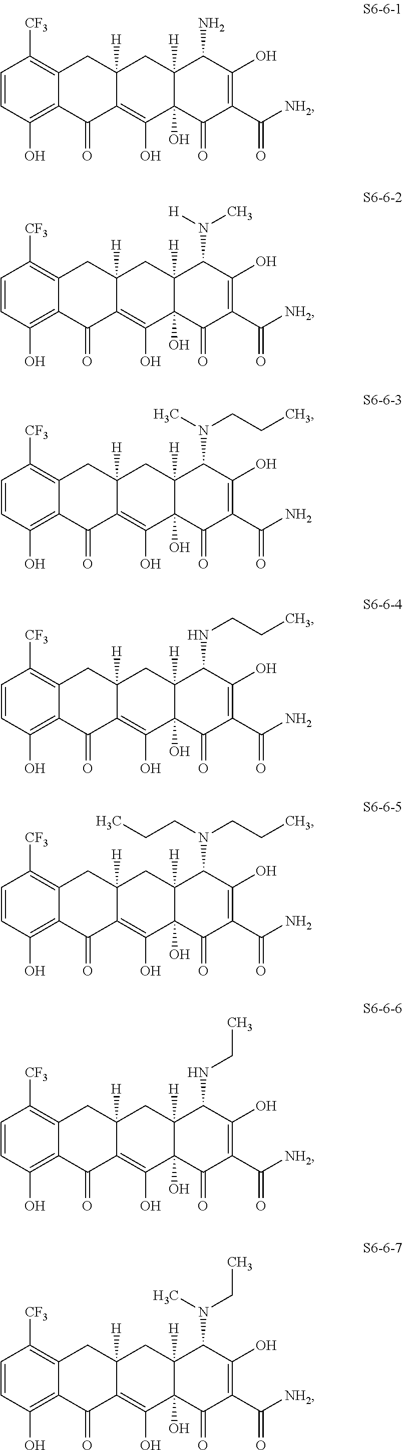

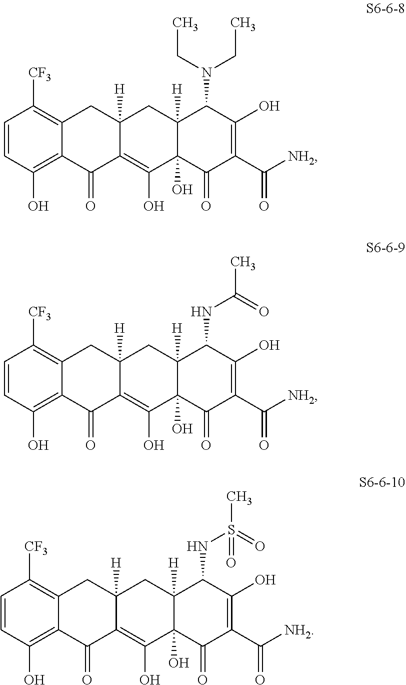 [SMILES: CCN(CC)[C@@H]1C(O)=C(C(N)=O)C(=O)[C@@]2(O)C(O)=C3C(=O)c4c(O)ccc(C(F)(F)F)c4C[C@H]3C[C@@H]12 ; CC(=O)N[C@@H]1C(O)=C(C(N)=O)C(=O)[C@@]2(O)C(O)=C3C(=O)c4c(O)ccc(C(F)(F)F)c4C[C@H]3C[C@@H]12 ; CS(=O)(=O)N[C@@H]1C(O)=C(C(N)=O)C(=O)[C@@]2(O)C(O)=C3C(=O)c4c(O)ccc(C(F)(F)F)c4C[C@H]3C[C@@H]12]

In the sixth aspect of the 32^{st} embodiment, the compound is represented by any one of the following structural formulas, or a pharmaceutically acceptable salt thereof:

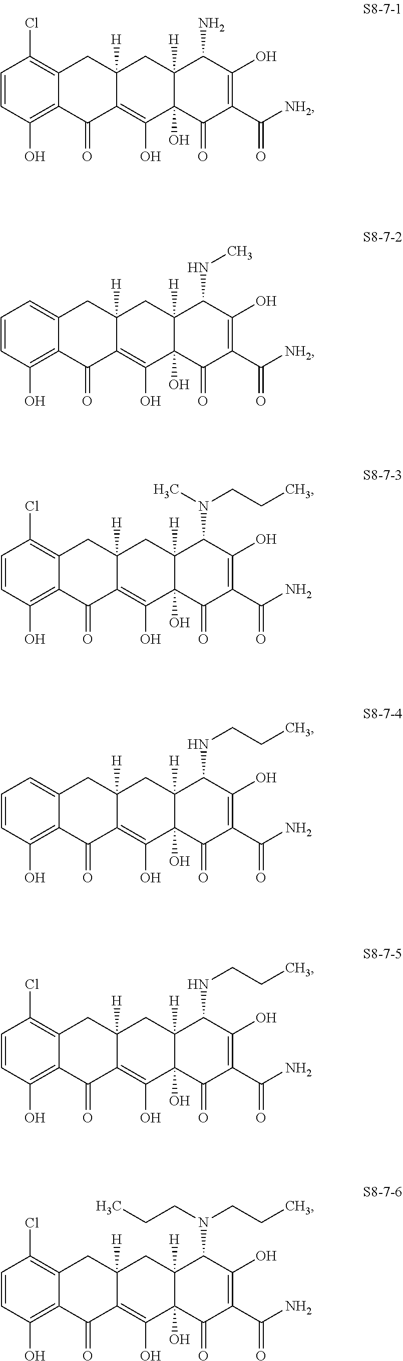 [SMILES: NC(=O)C1=C(O)[C@@H](N)[C@@H]2C[C@@H]3Cc4c(Cl)ccc(O)c4C(=O)C3=C(O)[C@]2(O)C1=O ; CN[C@@H]1C(O)=C(C(N)=O)C(=O)[C@@]2(O)C(O)=C3C(=O)c4c(O)cccc4C[C@H]3C[C@@H]12 ; CCCN(C)[C@@H]1C(O)=C(C(N)=O)C(=O)[C@@]2(O)C(O)=C3C(=O)c4c(O)ccc(Cl)c4C[C@H]3C[C@@H]12 ; CCCN[C@@H]1C(O)=C(C(N)=O)C(=O)[C@@]2(O)C(O)=C3C(=O)c4c(O)cccc4C[C@H]3C[C@@H]12 ; CCCN[C@@H]1C(O)=C(C(N)=O)C(=O)[C@@]2(O)C(O)=C3C(=O)c4c(O)ccc(Cl)c4C[C@H]3C[C@@H]12 ; CCCN(CCC)[C@@H]1C(O)=C(C(N)=O)C(=O)[C@@]2(O)C(O)=C3C(=O)c4c(O)ccc(Cl)c4C[C@H]3C[C@@H]12]

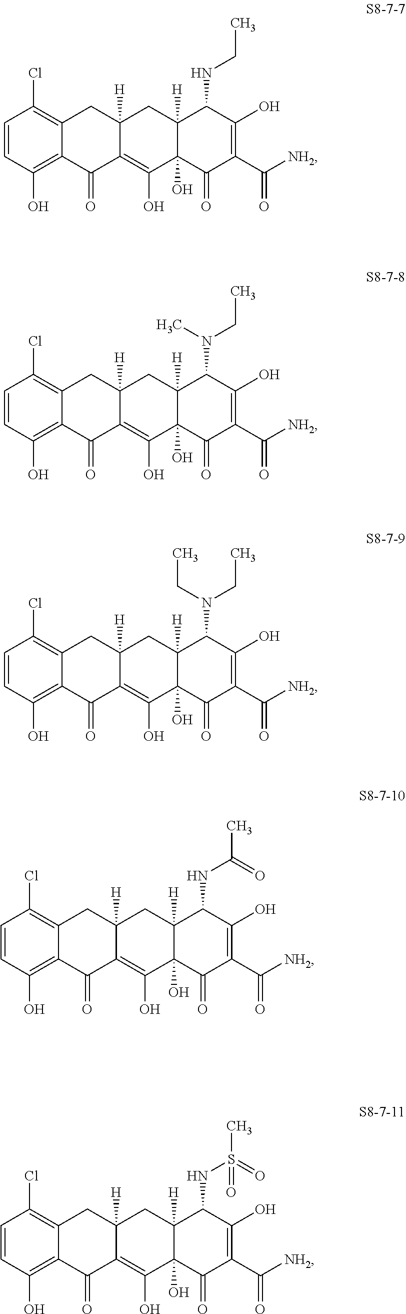 [SMILES: CCN[C@@H]1C(O)=C(C(N)=O)C(=O)[C@@]2(O)C(O)=C3C(=O)c4c(O)ccc(Cl)c4C[C@H]3C[C@@H]12 ; CCN(C)[C@@H]1C(O)=C(C(N)=O)C(=O)[C@@]2(O)C(O)=C3C(=O)c4c(O)ccc(Cl)c4C[C@H]3C[C@@H]12 ; CCN(CC)[C@@H]1C(O)=C(C(N)=O)C(=O)[C@@]2(O)C(O)=C3C(=O)c4c(O)ccc(Cl)c4C[C@H]3C[C@@H]12 ; CC(=O)N[C@@H]1C(O)=C(C(N)=O)C(=O)[C@@]2(O)C(O)=C3C(=O)c4c(O)ccc(Cl)c4C[C@H]3C[C@@H]12 ; CS(=O)(=O)N[C@@H]1C(O)=C(C(N)=O)C(=O)[C@@]2(O)C(O)=C3C(=O)c4c(O)ccc(Cl)c4C[C@H]3C[C@@H]12]

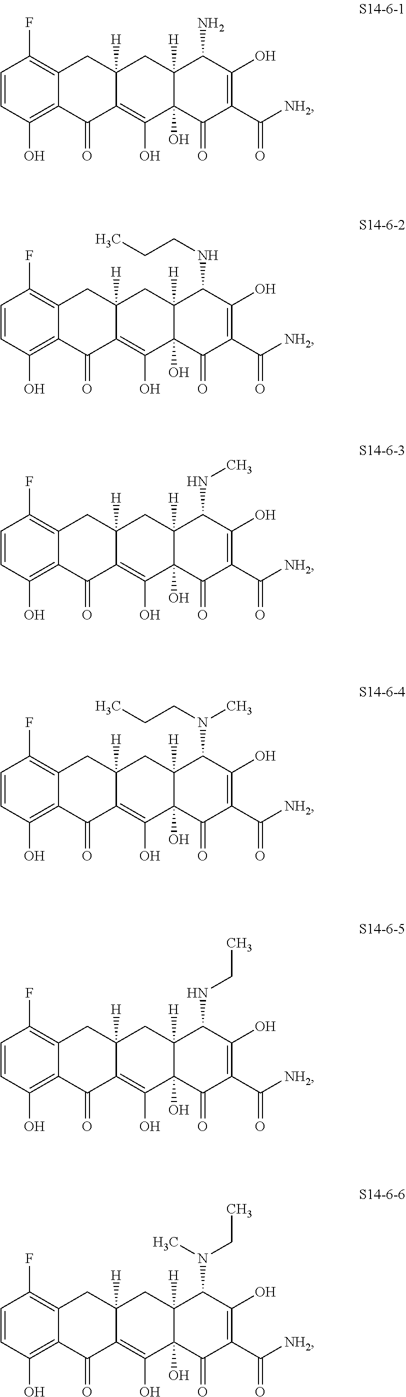 [SMILES: NC(=O)C1=C(O)[C@@H](N)[C@@H]2C[C@@H]3Cc4c(F)ccc(O)c4C(=O)C3=C(O)[C@]2(O)C1=O ; CCCN[C@@H]1C(O)=C(C(N)=O)C(=O)[C@@]2(O)C(O)=C3C(=O)c4c(O)ccc(F)c4C[C@H]3C[C@@H]12 ; CN[C@@H]1C(O)=C(C(N)=O)C(=O)[C@@]2(O)C(O)=C3C(=O)c4c(O)ccc(F)c4C[C@H]3C[C@@H]12 ; CCCN(C)[C@@H]1C(O)=C(C(N)=O)C(=O)[C@@]2(O)C(O)=C3C(=O)c4c(O)ccc(F)c4C[C@H]3C[C@@H]12 ; CCN[C@@H]1C(O)=C(C(N)=O)C(=O)[C@@]2(O)C(O)=C3C(=O)c4c(O)ccc(F)c4C[C@H]3C[C@@H]12 ; CCN(C)[C@@H]1C(O)=C(C(N)=O)C(=O)[C@@]2(O)C(O)=C3C(=O)c4c(O)ccc(F)c4C[C@H]3C[C@@H]12]

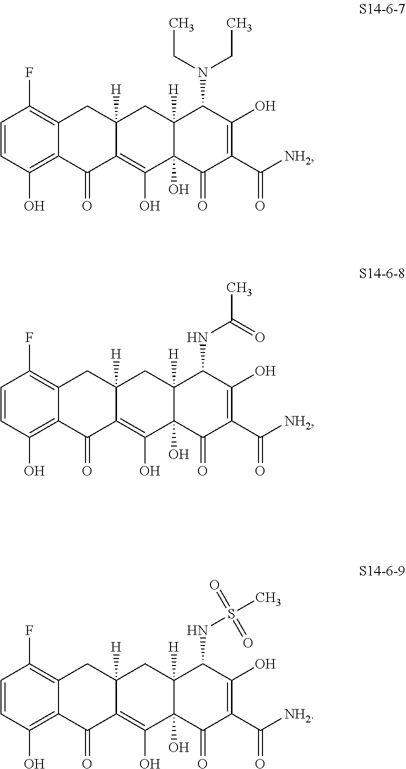 [SMILES: CCN(CC)[C@@H]1C(O)=C(C(N)=O)C(=O)[C@@]2(O)C(O)=C3C(=O)c4c(O)ccc(F)c4C[C@H]3C[C@@H]12 ; CC(=O)N[C@@H]1C(O)=C(C(N)=O)C(=O)[C@@]2(O)C(O)=C3C(=O)c4c(O)ccc(F)c4C[C@H]3C[C@@H]12 ; CS(=O)(=O)N[C@@H]1C(O)=C(C(N)=O)C(=O)[C@@]2(O)C(O)=C3C(=O)c4c(O)ccc(F)c4C[C@H]3C[C@@H]12]

In the seventh aspect of the 32^{st} embodiment, the compound is represented by any one of the following structural formulas, or a pharmaceutically acceptable salt thereof:

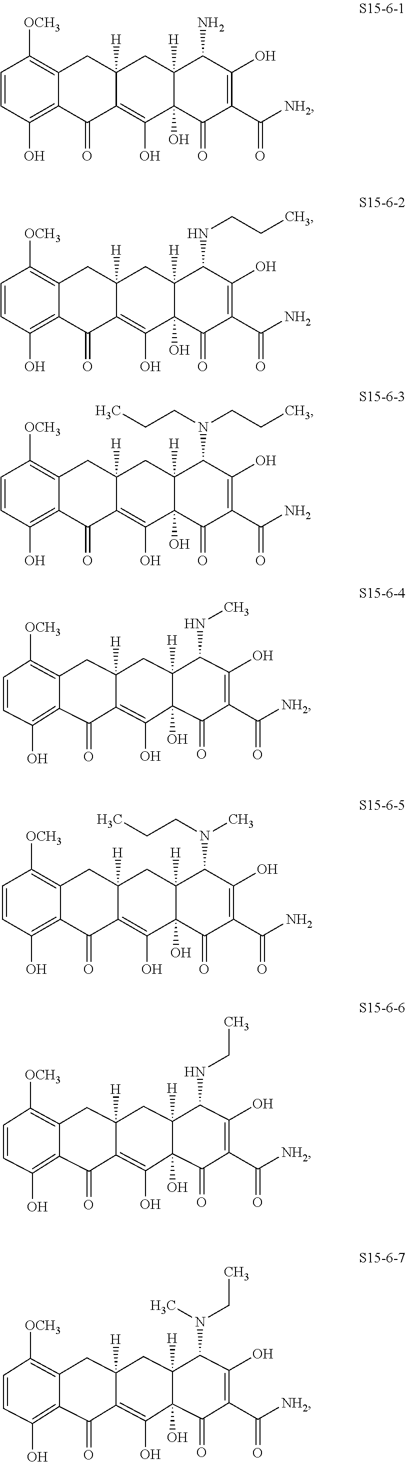

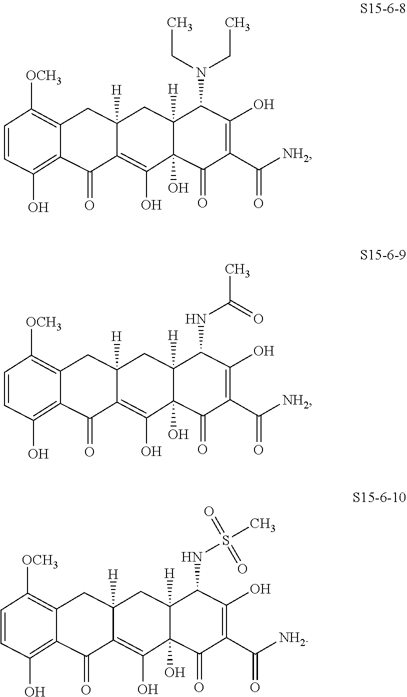 [SMILES: CCN(CC)[C@@H]1C(O)=C(C(N)=O)C(=O)[C@@]2(O)C(O)=C3C(=O)c4c(O)ccc(OC)c4C[C@H]3C[C@@H]12 ; COc1ccc(O)c2c1C[C@H]1C[C@H]3[C@H](NC(C)=O)C(O)=C(C(N)=O)C(=O)[C@@]3(O)C(O)=C1C2=O ; COc1ccc(O)c2c1C[C@H]1C[C@H]3[C@H](NS(C)(=O)=O)C(O)=C(C(N)=O)C(=O)[C@@]3(O)C(O)=C1C2=O]

In the 33^{rd} embodiment, the present invention is a method of treating a hematological cancer comprising administering to a subject in need of treatment an effective amount of a compound represented by any one of structural formulas

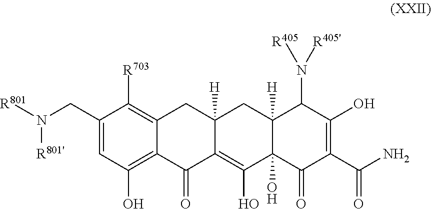

or a pharmaceutically acceptable salt thereof, or a pharmaceutically acceptable composition thereof. In a first aspect of the 33^{rd} embodiment, R⁷⁰³ is H, a halogen, a C₁₋₄ alkyloxy, —OH, C₁₋₄ alkyl, a C₁₋₄ haloalkyl, C₁₋₄ hydroxyalkyl, C₁₋₄ haloalkoxy, and R⁸⁰¹ and R^{801′} each independently is H, a C₁₋₆ alkyl, a C₃₋₁₂ carbocyclyl-(C₀₋₃)alkylenyl; and R⁴⁰⁵ and R^{405′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH(C₁₋₄ alkylenyl)-; a (C₁₋₄ alkyl)S(O)₁₋₂NH(C₁₋₄ alkylenyl)-; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 33^{rd} embodiment, R⁷⁰³ is a C₁₋₄ alkyloxy and R⁴⁰⁵ and R^{405′}, each independently, is H or a C₁₋₄ alkyl. The remainder of the values and example values of the variables in structural formula (XXII) of the 33^{rd} embodiment are as defined above with respect to aspect one of the 33^{rd} embodiment. Examples of the compounds of the 33^{rd} embodiment include compounds represented by any one of the following structural formulas, or a pharmaceutically acceptable salt thereof:

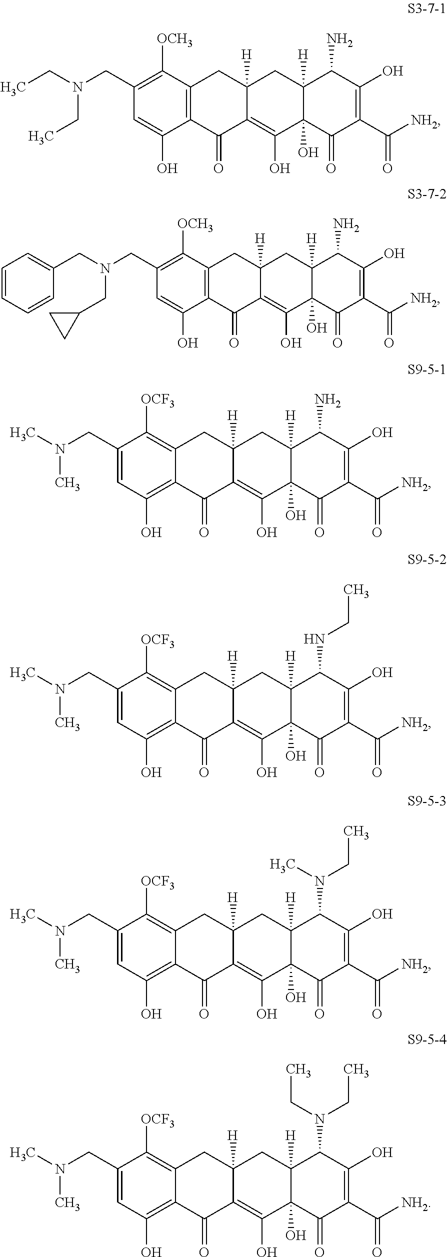

In a 34^{th} embodiment, the present invention is a method of treating a hematological cancer comprising administering to a subject in need of treatment an effective amount of a compound represented by the following structural formula

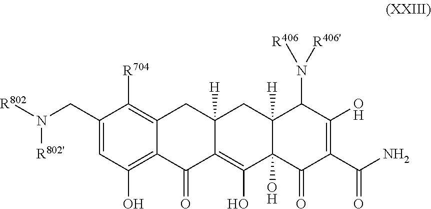

or a pharmaceutically acceptable salt thereof, or a pharmaceutically acceptable composition thereof. In a first aspect of the 34^{th} embodiment, R⁷⁰⁴ is H, a halogen, a C₁₋₄ alkyloxy, —OH, C₁₋₄ alkyl, a C₁₋₄ haloalkyl, C₁₋₄ hydroxyalkyl, C₁₋₄ haloalkoxy; R⁸⁰² and R^{802′}, taken together with the nitrogen atom to which they are attached, form a 4-13 monocyclic or a 7-13 bicyclic heterocyclyl; and R⁴⁰⁶ and R^{406′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH(C₁₋₄ alkylenyl)-; a (C₁₋₄ alkyl)S(O)₁₋₂NH(C₁₋₄ alkylenyl)-; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 34^{th} embodiment, R⁷⁰⁴ is a halogen; and R⁸⁰² and R^{802′}, taken together with the nitrogen atom to which they are attached, form 1,2,3,4-tetrahydroisoquinoline. The remainder of the values and example values of the variables in structural formula (XXIII) of the 34^{th} embodiment are as defined above with respect to aspect one of the 34^{th} embodiment.

Examples of the compounds of the 34^{th} embodiment include compounds represented by any one of the following structural formulas, or a pharmaceutically acceptable salt thereof:

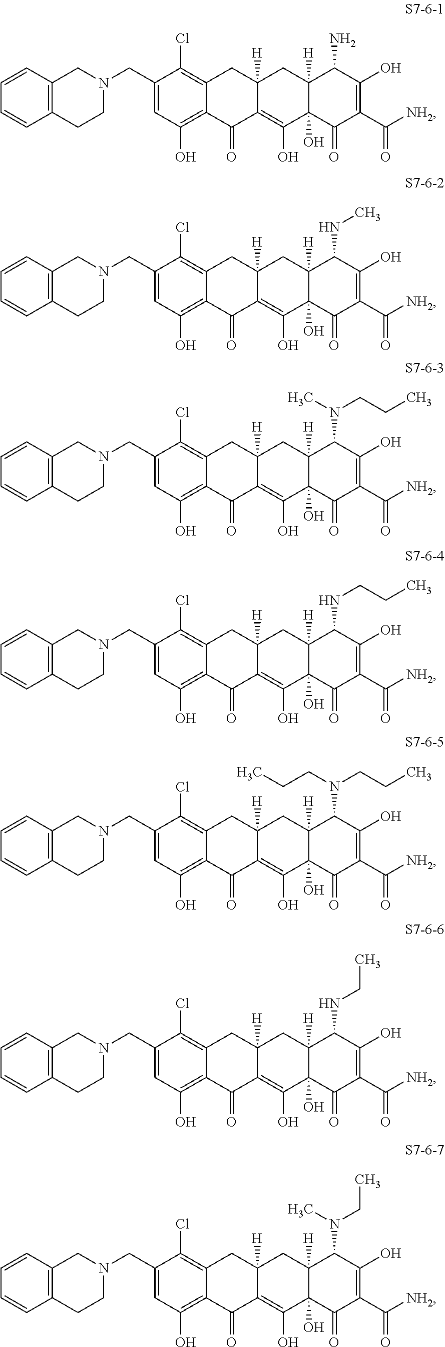

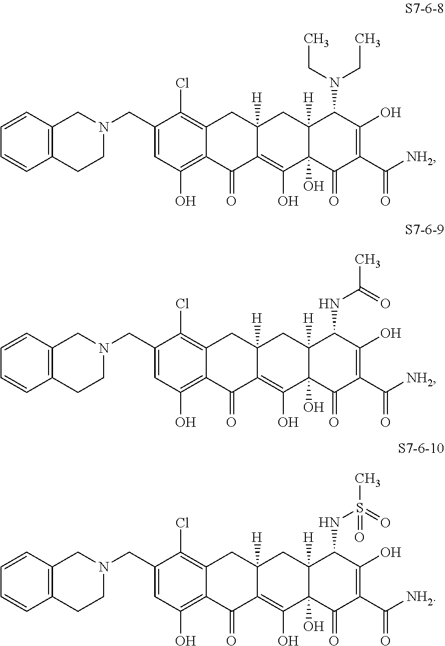 [SMILES: CCN(CC)[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12 ; CC(=O)N[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12 ; CS(=O)(=O)N[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12]

In the 35^{th} embodiment, the present invention is a method of treating a hematological cancer comprising administering to a subject in need of treatment an effective amount of a compound represented by the following structural formula

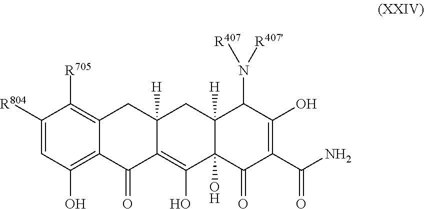

or a pharmaceutically acceptable salt thereof, or a pharmaceutically acceptable composition thereof. In a first aspect of the 35^{th} embodiment, R⁷⁰⁵ is H, a halogen, a C₁₋₄ alkyloxy, —OH, C₁₋₄ alkyl, a C₁₋₄ haloalkyl, C₁₋₄ hydroxyalkyl, or C₁₋₄ haloalkoxy; R⁸⁰⁴ is an amino-C₁₋₆ alkyl, a mono- or di-(C₁-C₄ alkyl)amino(C₁₋₆)alkyl, or, a C-attached 4-13 monocyclyc heterocyclyl, wherein the hetrocyclyl is optionally N-substituted with a C₁₋₄ alkyl; and R⁴⁰⁷ and R^{407′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH(C₁₋₄ alkylenyl)-; a (C₁₋₄ alkyl)S(O)₁₋₂NH(C₁₋₄ alkylenyl)-; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 35^{th} embodiment, R⁷⁰⁵ is a C₁₋₄ haloalkyl; and R⁸⁰⁴ is a mono- or di-(C₁-C₂ alkyl)amino(C₁₋₆)alkyl. The remainder of the values and example values of the variables in structural formula (XXIV) of the 35^{th} embodiment are as defined above with respect to aspect one of the 35^{th} embodiment.

In the third aspect of the 35^{th} embodiment, R⁷⁰⁵ is a C₁₋₄ haloalkyl; and R⁸⁰⁴ is a 4-5 monocyclyc heterocyclyl, N-substituted with methyl or ethyl. The remainder of the values and example values of the variables in structural formula (XXIV) of the 35^{th} embodiment are as defined above with respect to aspect one of the 35^{th} embodiment.

In the fourth aspect of the 35^{th} embodiment, the compound is represented by any one of the following structural formulas, or a pharmaceutically acceptable salt thereof:

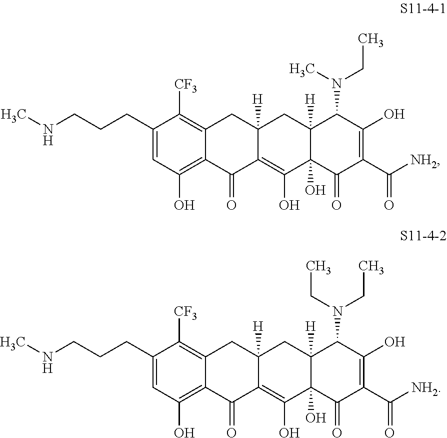 [SMILES: CCN(C)[C@@H]1C(O)=C(C(N)=O)C(=O)[C@@]2(O)C(O)=C3C(=O)c4c(O)cc(CCCNC)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc(CCCNC)c(C(F)(F)F)c4C[C@H]3C[C@@H]12]

In the fifth aspect of the 35^{th} embodiment, the compound is represented by any one of the following structural formulas, or a pharmaceutically acceptable salt thereof:

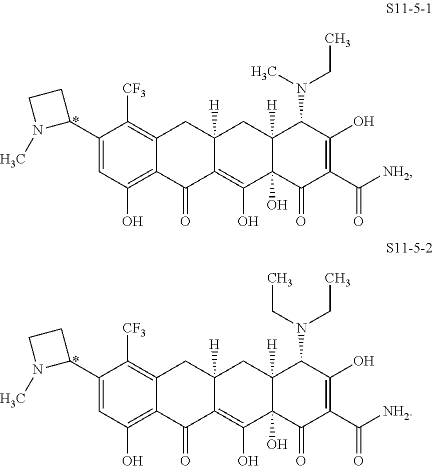 [SMILES: CCN(C)[C@@H]1C(O)=C(C(N)=O)C(=O)[C@@]2(O)C(O)=C3C(=O)c4c(O)cc(C5CCN5C)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc(C5CCN5C)c(C(F)(F)F)c4C[C@H]3C[C@@H]12]

In the 36^{th} embodiment, the present invention is a method of treating a hematological cancer comprising administering to a subject in need of treatment an effective amount of a compound represented by the following structural formula

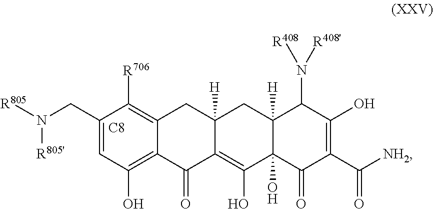

or a pharmaceutically acceptable salt thereof, or a pharmaceutically acceptable composition thereof. In a first aspect of the 36^{th} embodiment, R⁷⁰⁶ is H, a halogen, a C₁₋₄ alkyloxy, —OH, C₁₋₄ alkyl, a C₁₋₄ haloalkyl, C₁₋₄ hydroxyalkyl, or C₁₋₄ haloalkoxy; R⁸⁰⁵ and R^{805′}, taken together with the nitrogen atom to which they are attached, form a 4-13 monocyclyc heterocyclyl optionally substituted with a C₃₋₁₂ carbocyclyl; and R⁴⁰⁸ and R^{408′}, each independently, is H; a C₁₋₄ alkyl; a C₁-C₄ haloalkyl; a C₁-C₄ hydroxyalkyl; a (C₁-C₄ alkoxy)-(C₁₋₄)alkyl; an amino-(C₁-C₄) alkyl; a mono- or di-(C₁-C₄ alkyl)amino-(C₁₋₄)alkyl; a C₃₋₁₂ carbocyclyl-(C₀-C₃)alkylenyl, wherein the carbocyclyl portion is optionally substituted with a hydroxyl group; a (C₁₋₄ alkyl)C(O)—, a (C₁₋₄ alkyl)S(O)₁₋₂—; a (C₁₋₄ alkyl)C(O)NH(C₁₋₄ alkylenyl)-; a (C₁₋₄ alkyl)S(O)₁₋₂NH(C₁₋₄ alkylenyl)-; a HOC(O)—(C₁-C₃)alkylenyl; a H₂NC(O)—(C₁-C₃)alkylenyl; a (C₁₋₄ alkyloxy)C(O)—(C₁-C₃)alkylenyl.

In the second aspect of the 36^{th} embodiment, R⁷⁰⁶ is a halogen, and R⁸⁰⁵ and R^{805′}, taken together with the nitrogen atom to which they are attached, form a 5-6 monocyclyc heterocyclyl optionally substituted with a phenyl. The remainder of the values and example values of the variables in structural formula (XXV) of the 36^{th} embodiment are as defined above with respect to aspect one of the 36^{th} embodiment.

Example embodiments of the 36^{th} embodiment include the compound is represented by any one of the following structural formulas, or a pharmaceutically acceptable salt thereof:

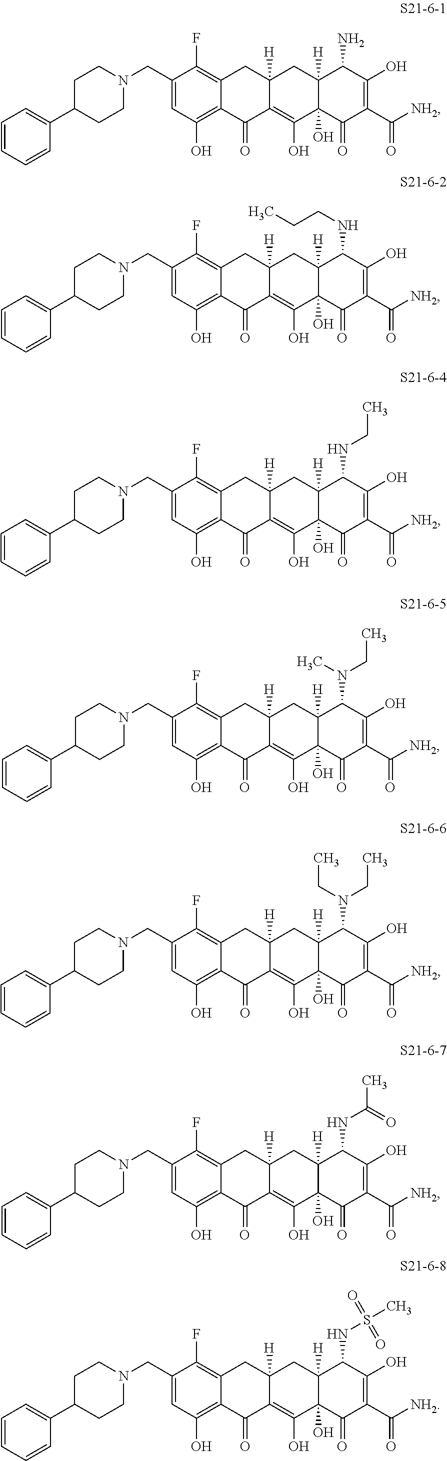

In the 37^{th} embodiment, the present invention is any compound represented by structural formula (XIII):

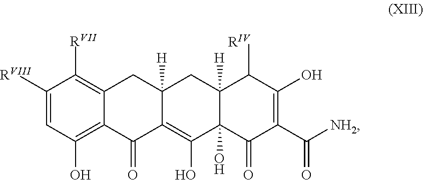

or a pharmaceutically acceptable salt thereof, or a pharmaceutically acceptable composition thereof. In the first aspect of the 37^{th} embodiment,

| Compound | | | |
|---|---|---|---|
| number | R^{IV} | R^{VII} | R^{VIII} |
| S8-7-2 | 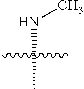 | 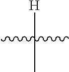 [SMILES: *[H]] | 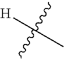 [SMILES: *[H]] |
| S8-7-4 | 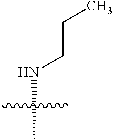 | 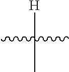 [SMILES: *[H]] | 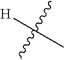 [SMILES: *[H]] |
| S15-6-1 | 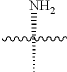 | 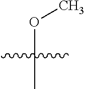 [SMILES: *OC] | 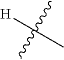 [SMILES: *[H]] |
| S15-6-4 | 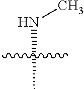 | 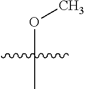 [SMILES: *OC] | 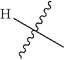 [SMILES: *[H]] |
| S15-6-6 | 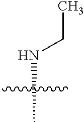 | 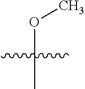 [SMILES: *OC] | 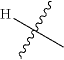 [SMILES: *[H]] |
| S15-6-2 | 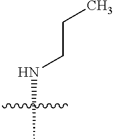 | 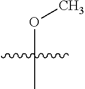 [SMILES: *OC] | 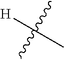 [SMILES: *[H]] |
| S15-6-7 | 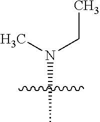 | 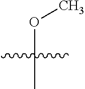 [SMILES: *OC] | 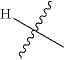 [SMILES: *[H]] |
| S15-6-8 | 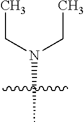 | 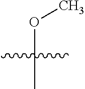 [SMILES: *OC] | 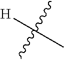 [SMILES: *[H]] |
| S15-6-5 | 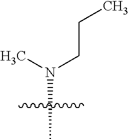 | 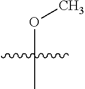 [SMILES: *OC] | 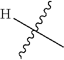 [SMILES: *[H]] |
| S15-6-3 | 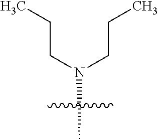 | 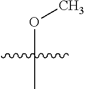 [SMILES: *OC] | 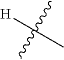 [SMILES: *[H]] |
| S15-6-9 | 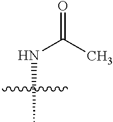 | 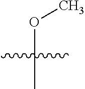 [SMILES: *OC] | 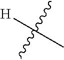 [SMILES: *[H]] |
| S15-6-10 | 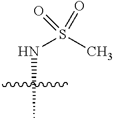 | 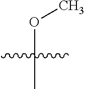 [SMILES: *OC] | 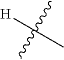 [SMILES: *[H]] |
| S2-9-6 | 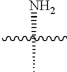 | 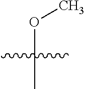 [SMILES: *OC] | 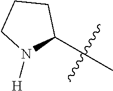 [SMILES: *[C@@H]1CCCN1] |
| S2-9-5 | 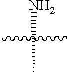 | 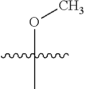 [SMILES: *OC] | 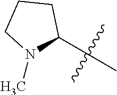 [SMILES: *[C@@H]1CCCN1C] |
| S1-7-3 | 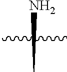 | 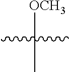 [SMILES: *OC] | 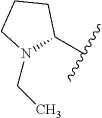 [SMILES: *[C@H]1CCCN1CC] |
| S1-7-1 | 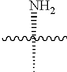 | 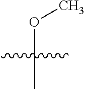 [SMILES: *OC] | 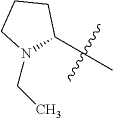 [SMILES: *[C@H]1CCCN1CC] |
| S1-7-2 | 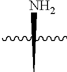 | 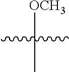 [SMILES: *OC] | 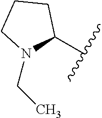 [SMILES: *[C@@H]1CCCN1CC] |
| S1-6-2 | 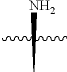 | 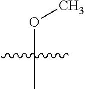 [SMILES: *OC] | 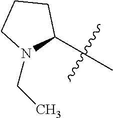 [SMILES: *[C@@H]1CCCN1CC] |
| S2-9-3 | 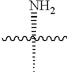 | 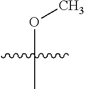 [SMILES: *OC] | 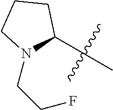 [SMILES: *[C@@H]1CCCN1CCF] |
| S2-9-1 | 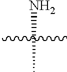 | 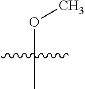 [SMILES: *OC] | 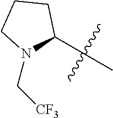 [SMILES: *[C@@H]1CCCN1CC(F)(F)F] |
| S2-9-9 | 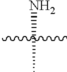 | 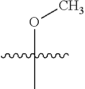 [SMILES: *OC] | 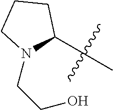 [SMILES: *[C@@H]1CCCN1CCO] |
| S2-9-16 | 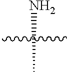 | 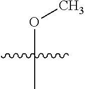 [SMILES: *OC] | 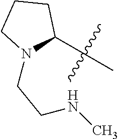 [SMILES: *[C@@H]1CCCN1CCNC] |
| S2-9-7 | 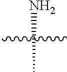 | 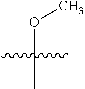 [SMILES: *OC] | 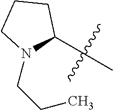 [SMILES: *[C@@H]1CCCN1CCC] |
| S2-9-10 | 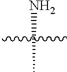 | 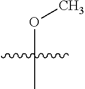 [SMILES: *OC] | 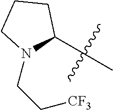 [SMILES: *[C@@H]1CCCN1CCC(F)(F)F] |
| S2-9-8 | 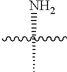 | 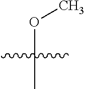 [SMILES: *OC] | 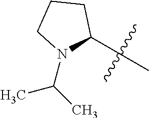 [SMILES: *[C@@H]1CCCN1C(C)C] |
| S2-9-13 | 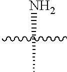 | 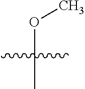 [SMILES: *OC] | 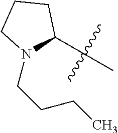 [SMILES: *[C@@H]1CCCN1CCCC] |
| S2-9-14 | 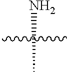 | 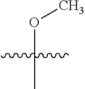 [SMILES: *OC] | 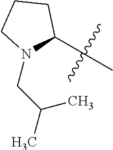 [SMILES: *[C@@H]1CCCN1CC(C)C] |
| S2-9-11 | 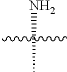 | 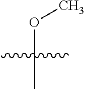 [SMILES: *OC] | 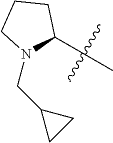 [SMILES: *[C@@H]1CCCN1CC1CC1] |
| S2-9-15 | 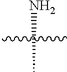 | 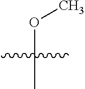 [SMILES: *OC] | 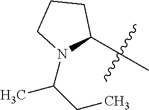 [SMILES: *[C@@H]1CCCN1C(C)CC] |
| S2-9-18 | 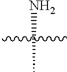 | 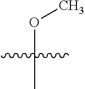 [SMILES: *OC] | 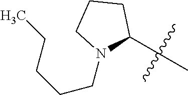 [SMILES: *[C@@H]1CCCN1CCCCC] |
| S2-9-19 | 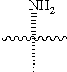 | 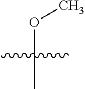 [SMILES: *OC] | 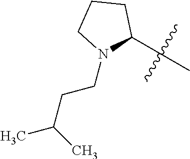 [SMILES: *[C@@H]1CCCN1CCC(C)C] |
| S2-9-20 | 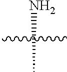 | 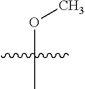 [SMILES: *OC] | 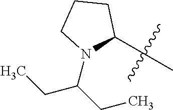 [SMILES: *[C@@H]1CCCN1C(CC)CC] |
| S2-9-21 | 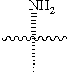 | 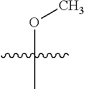 [SMILES: *OC] | 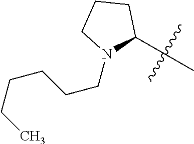 [SMILES: *[C@@H]1CCCN1CCCCCC] |
| S2-9-2 | 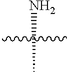 | 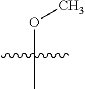 [SMILES: *OC] | 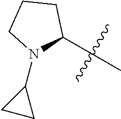 [SMILES: *[C@@H]1CCCN1C1CC1] |
| S2-9-12 | 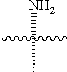 | 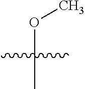 [SMILES: *OC] | 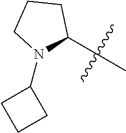 [SMILES: *[C@@H]1CCCN1C1CCC1] |
| S2-9-17 | 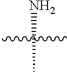 | 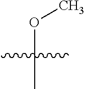 [SMILES: *OC] | 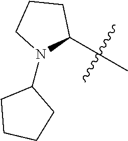 [SMILES: *[C@@H]1CCCN1C1CCCC1] |
| S2-9-22 | 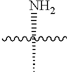 | 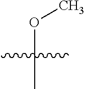 [SMILES: *OC] | 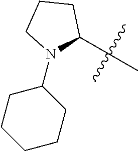 [SMILES: *[C@@H]1CCCN1C1CCCCC1] |
| S2-9-28 | 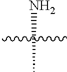 | 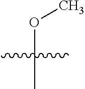 [SMILES: *OC] | 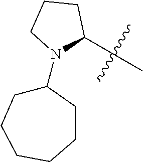 [SMILES: *[C@@H]1CCCN1C1CCCCCC1] |
| S2-9-29 | 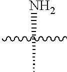 | 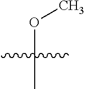 [SMILES: *OC] | 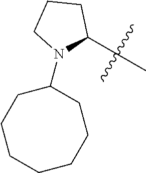 [SMILES: *[C@@H]1CCCN1C1CCCCCCC1] |
| S2-9-23 | 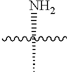 | 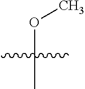 [SMILES: *OC] | 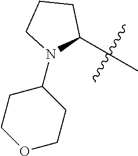 [SMILES: *[C@@H]1CCCN1C1CCOCC1] |
| S2-9-24 | 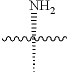 | 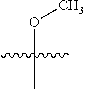 [SMILES: *OC] | 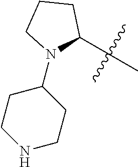 [SMILES: *[C@@H]1CCCN1C1CCNCC1] |
| S2-9-25 | 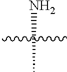 | 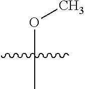 [SMILES: *OC] | 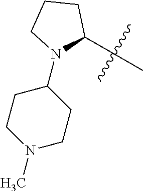 [SMILES: *[C@@H]1CCCN1C1CCN(C)CC1] |
| S2-9-26 | 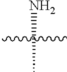 | 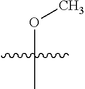 [SMILES: *OC] | 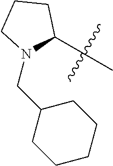 [SMILES: *[C@@H]1CCCN1CC1CCCCC1] |
| S2-9-4 | 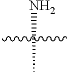 | 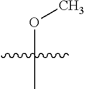 [SMILES: *OC] | 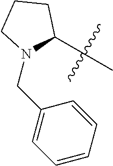 [SMILES: *[C@@H]1CCCN1Cc1ccccc1] |
| S2-9-27 | 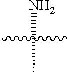 | 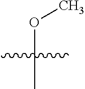 [SMILES: *OC] | 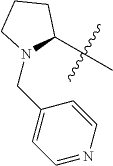 [SMILES: *[C@@H]1CCCN1Cc1ccncc1] |
| S1-5-9 | 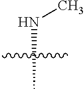 | 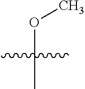 [SMILES: *OC] | 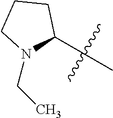 [SMILES: *[C@@H]1CCCN1CC] |
| S1-6-1 | 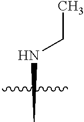 | 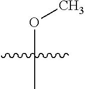 [SMILES: *OC] | 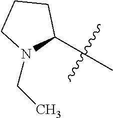 [SMILES: *[C@@H]1CCCN1CC] |
| S1-5-1 | 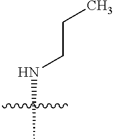 | 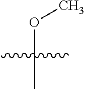 [SMILES: *OC] | 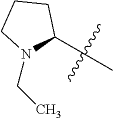 [SMILES: *[C@@H]1CCCN1CC] |
| S1-5-2 | 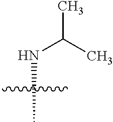 | 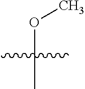 [SMILES: *OC] | 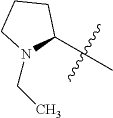 [SMILES: *[C@@H]1CCCN1CC] |
| S1-5-8 | 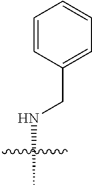 | 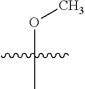 [SMILES: *OC] | 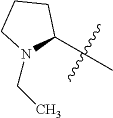 [SMILES: *[C@@H]1CCCN1CC] |
| S1-5-4 | 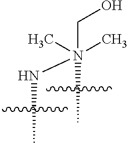 | 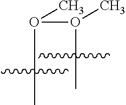 | 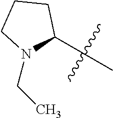 [SMILES: *[C@@H]1CCCN1CC] |
| S1-5-6 | 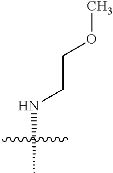 | 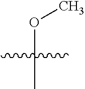 [SMILES: *OC] | 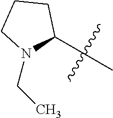 [SMILES: *[C@@H]1CCCN1CC] |
| S1-5-5 | 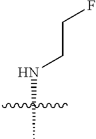 | 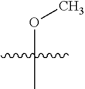 [SMILES: *OC] | 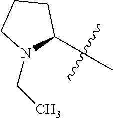 [SMILES: *[C@@H]1CCCN1CC] |
| S1-5-3 | 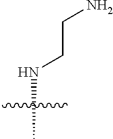 | 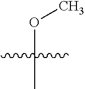 [SMILES: *OC] | 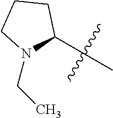 [SMILES: *[C@@H]1CCCN1CC] |
| S1-5-7 | 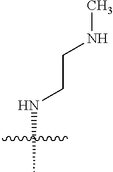 | 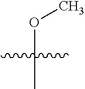 [SMILES: *OC] | 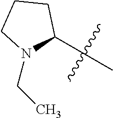 [SMILES: *[C@@H]1CCCN1CC] |
| S1-5-14 | 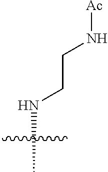 | 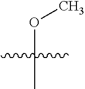 [SMILES: *OC] | 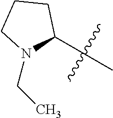 [SMILES: *[C@@H]1CCCN1CC] |
| S1-5-15 | 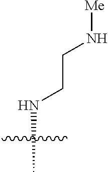 | 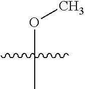 [SMILES: *OC] | 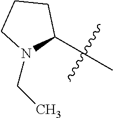 [SMILES: *[C@@H]1CCCN1CC] |
| S1-5-18 | 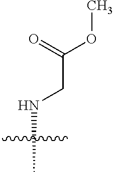 | 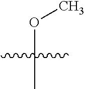 [SMILES: *OC] | 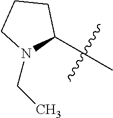 [SMILES: *[C@@H]1CCCN1CC] |
| S1-5-17 | 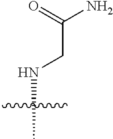 | 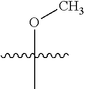 [SMILES: *OC] | 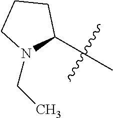 [SMILES: *[C@@H]1CCCN1CC] |
| S1-5-16 | 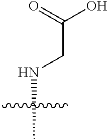 | 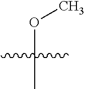 [SMILES: *OC] | 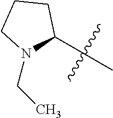 [SMILES: *[C@@H]1CCCN1CC] |
| S1-5-10 | 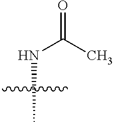 | 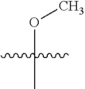 [SMILES: *OC] | 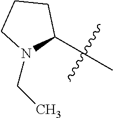 [SMILES: *[C@@H]1CCCN1CC] |
| S1-5-12 | 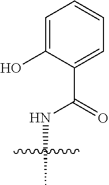 | 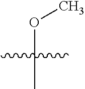 [SMILES: *OC] | 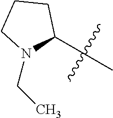 [SMILES: *[C@@H]1CCCN1CC] |
| S1-5-11 | 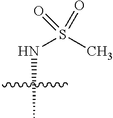 | 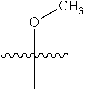 [SMILES: *OC] | 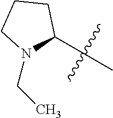 [SMILES: *[C@@H]1CCCN1CC] |
| S1-5-13 | 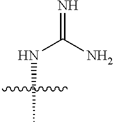 | 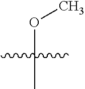 [SMILES: *OC] | 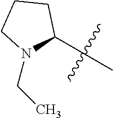 [SMILES: *[C@@H]1CCCN1CC] |
| S4-7-1 | 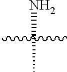 | 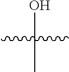 [SMILES: *O] | 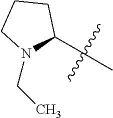 [SMILES: *[C@@H]1CCCN1CC] |
| S4-7-3 | 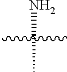 | 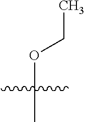 [SMILES: *OCC] | 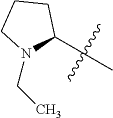 [SMILES: *[C@@H]1CCCN1CC] |
| S4-7-4 | 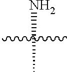 | 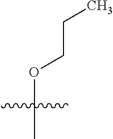 [SMILES: *OCCC] | 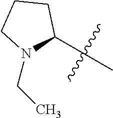 [SMILES: *[C@@H]1CCCN1CC] |
| S4-7-5 | 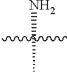 | 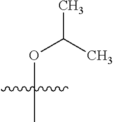 [SMILES: *OC(C)C] | 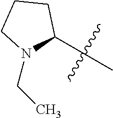 [SMILES: *[C@@H]1CCCN1CC] |
| S4-7-6 | 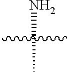 | 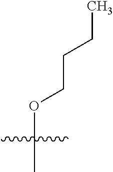 [SMILES: *OCCCC] | 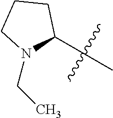 [SMILES: *[C@@H]1CCCN1CC] |
| S4-7-2 | 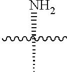 | 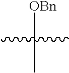 [SMILES: *OCc1ccccc1] | 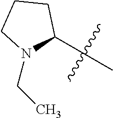 [SMILES: *[C@@H]1CCCN1CC] |
| S3-7-1 | 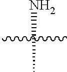 | 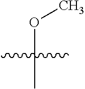 [SMILES: *OC] | 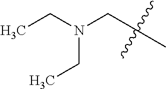 [SMILES: *CN(CC)CC] |
| S3-7-2 | 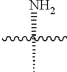 | 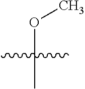 [SMILES: *OC] | 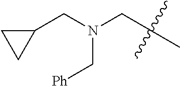 [SMILES: *CN(Cc1ccccc1)CC1CC1] |
| S9-5-1 | 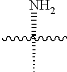 | 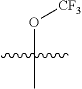 [SMILES: *OC(F)(F)F] | 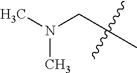 [SMILES: *CN(C)C] |
| S9-5-2 | 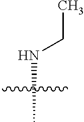 | 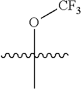 [SMILES: *OC(F)(F)F] | 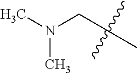 [SMILES: *CN(C)C] |
| S9-5-3 | 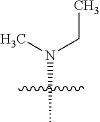 | 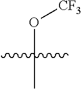 [SMILES: *OC(F)(F)F] | 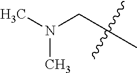 [SMILES: *CN(C)C] |
| S9-5-4 | 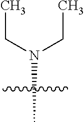 | 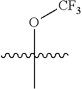 [SMILES: *OC(F)(F)F] | 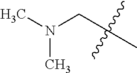 [SMILES: *CN(C)C] |
| S10-5-1 | 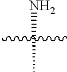 | 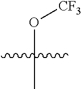 [SMILES: *OC(F)(F)F] | 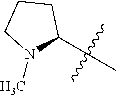 [SMILES: *[C@@H]1CCCN1C] |
| S10-5-2 | 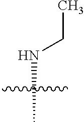 | 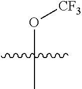 [SMILES: *OC(F)(F)F] | 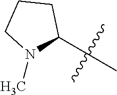 [SMILES: *[C@@H]1CCCN1C] |
| S10-5-3 | 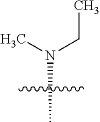 | 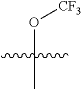 [SMILES: *OC(F)(F)F] | 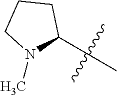 [SMILES: *[C@@H]1CCCN1C] |
| S10-5-4 | 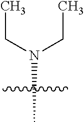 | 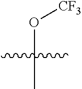 [SMILES: *OC(F)(F)F] | 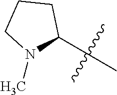 [SMILES: *[C@@H]1CCCN1C] |
| S8-7-1 | 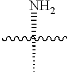 | 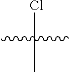 [SMILES: *Cl] | 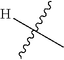 [SMILES: *[H]] |
| S8-7-7 | 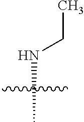 | 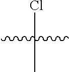 [SMILES: *Cl] | 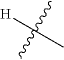 [SMILES: *[H]] |
| S8-7-5 | 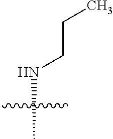 | 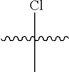 [SMILES: *Cl] | 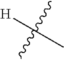 [SMILES: *[H]] |
| S8-7-8 | 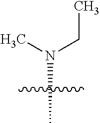 | 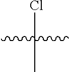 [SMILES: *Cl] | 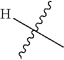 [SMILES: *[H]] |
| S8-7-9 | 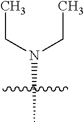 | 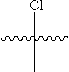 [SMILES: *Cl] | 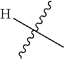 [SMILES: *[H]] |
| S8-7-3 | 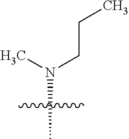 | 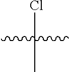 [SMILES: *Cl] | 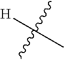 [SMILES: *[H]] |
| S8-7-6 | 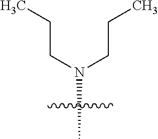 | 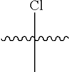 [SMILES: *Cl] | 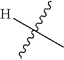 [SMILES: *[H]] |
| S8-7-10 | 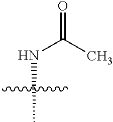 | 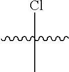 [SMILES: *Cl] | 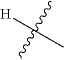 [SMILES: *[H]] |
| S8-7-11 | 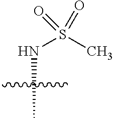 | 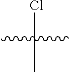 [SMILES: *Cl] | 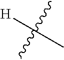 [SMILES: *[H]] |
| S7-6-1 | 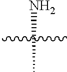 | 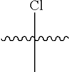 [SMILES: *Cl] | 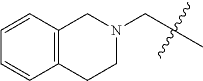 [SMILES: *CN1CCc2ccccc2C1] |
| S7-6-2 | 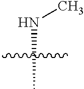 | 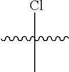 [SMILES: *Cl] | 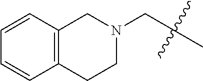 [SMILES: *CN1CCc2ccccc2C1] |
| S7-6-6 | 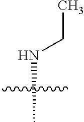 | 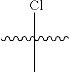 [SMILES: *Cl] | 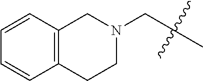 [SMILES: *CN1CCc2ccccc2C1] |
| S7-6-4 | 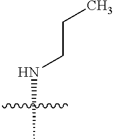 | 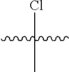 [SMILES: *Cl] | 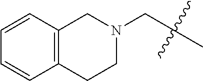 [SMILES: *CN1CCc2ccccc2C1] |
| S7-6-7 | 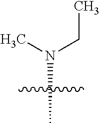 | 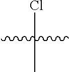 [SMILES: *Cl] | 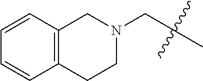 [SMILES: *CN1CCc2ccccc2C1] |
| S7-6-8 | 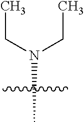 | 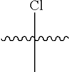 [SMILES: *Cl] | 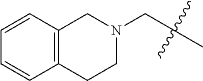 [SMILES: *CN1CCc2ccccc2C1] |
| S7-6-3 | 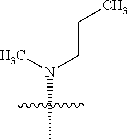 | 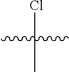 [SMILES: *Cl] | 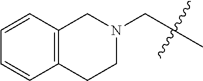 [SMILES: *CN1CCc2ccccc2C1] |
| S7-6-5 | 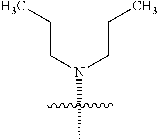 | 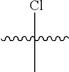 [SMILES: *Cl] | 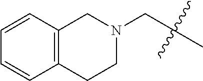 [SMILES: *CN1CCc2ccccc2C1] |
| S7-6-9 | 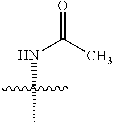 | 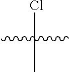 [SMILES: *Cl] | 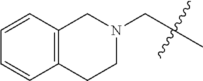 [SMILES: *CN1CCc2ccccc2C1] |
| S7-6-10 | 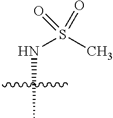 | 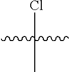 [SMILES: *Cl] | 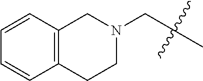 [SMILES: *CN1CCc2ccccc2C1] |
| S6-6-1 | 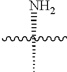 | 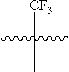 [SMILES: *C(F)(F)F] | 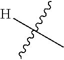 [SMILES: *[H]] |
| S6-6-2 | 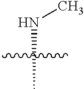 | 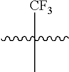 [SMILES: *C(F)(F)F] | 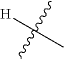 [SMILES: *[H]] |
| S6-6-6 | 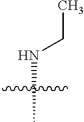 | 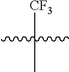 [SMILES: *C(F)(F)F] | 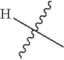 [SMILES: *[H]] |
| S6-6-7 | 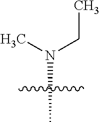 | 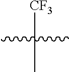 [SMILES: *C(F)(F)F] | 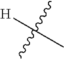 [SMILES: *[H]] |
| S6-6-3 | 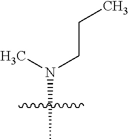 | 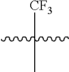 [SMILES: *C(F)(F)F] | 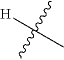 [SMILES: *[H]] |
| S6-6-8 | 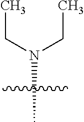 | 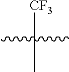 [SMILES: *C(F)(F)F] | 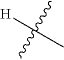 [SMILES: *[H]] |
| S6-6-5 | 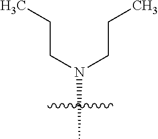 | 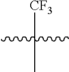 [SMILES: *C(F)(F)F] | 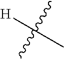 [SMILES: *[H]] |
| S6-6-4 | 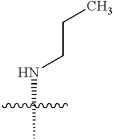 | 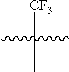 [SMILES: *C(F)(F)F] | 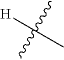 [SMILES: *[H]] |
| S6-6-9 | 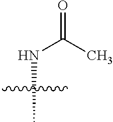 | 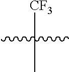 [SMILES: *C(F)(F)F] | 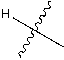 [SMILES: *[H]] |
| S6-6-10 | 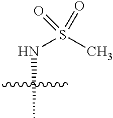 | 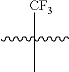 [SMILES: *C(F)(F)F] | 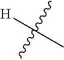 [SMILES: *[H]] |
| S11-4-1 | 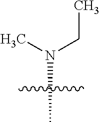 | 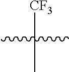 [SMILES: *C(F)(F)F] | 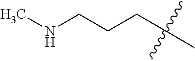 [SMILES: *CCCNC] |
| S11-4-2 | 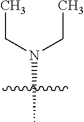 | 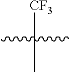 [SMILES: *C(F)(F)F] | 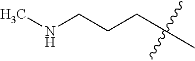 [SMILES: *CCCNC] |
| S11-5-1 | 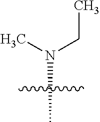 | 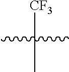 [SMILES: *C(F)(F)F] | 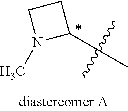 [SMILES: *C1CCN1C] |
| S11-5-2 | 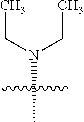 | 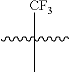 [SMILES: *C(F)(F)F] | 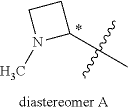 [SMILES: *C1CCN1C] |
| S12-2-1 | 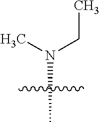 | 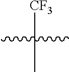 [SMILES: *C(F)(F)F] | 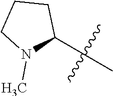 [SMILES: *[C@@H]1CCCN1C] |
| S12-2-2 | 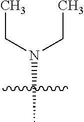 | 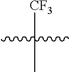 [SMILES: *C(F)(F)F] | 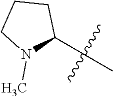 [SMILES: *[C@@H]1CCCN1C] |
| S12-2-3 | 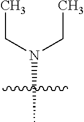 | 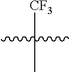 [SMILES: *C(F)(F)F] | 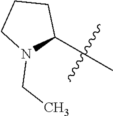 [SMILES: *[C@@H]1CCCN1CC] |
| S12-2-4 | 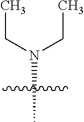 | 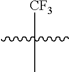 [SMILES: *C(F)(F)F] | 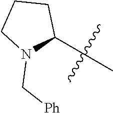 [SMILES: *[C@@H]1CCCN1Cc1ccccc1] |
| S14-6-1 | 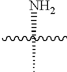 | 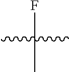 [SMILES: *F] | 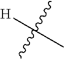 [SMILES: *[H]] |
| S14-6-3 | 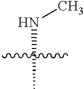 | 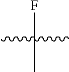 [SMILES: *F] | 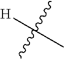 [SMILES: *[H]] |
| S14-6-5 | 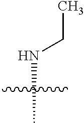 | 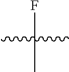 [SMILES: *F] | 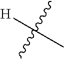 [SMILES: *[H]] |
| S14-6-2 | 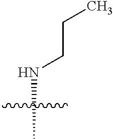 | 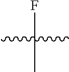 [SMILES: *F] | 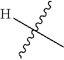 [SMILES: *[H]] |
| S14-6-6 | 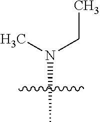 | 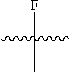 [SMILES: *F] | 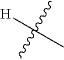 [SMILES: *[H]] |
| S14-6-4 | 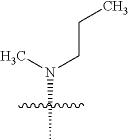 | 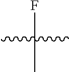 [SMILES: *F] | 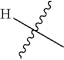 [SMILES: *[H]] |
| S14-6-7 | 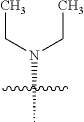 | 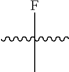 [SMILES: *F] | 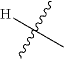 [SMILES: *[H]] |
| S14-6-8 | 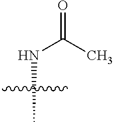 | 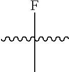 [SMILES: *F] | 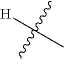 [SMILES: *[H]] |
| S14-6-9 | 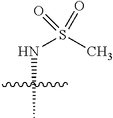 | 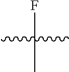 [SMILES: *F] | 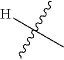 [SMILES: *[H]] |
| S21-6-1 | 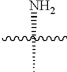 | 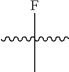 [SMILES: *F] | 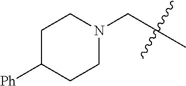 [SMILES: *CN1CCC(c2ccccc2)CC1] |
| S21-6-3 | 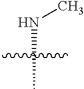 | 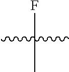 [SMILES: *F] | 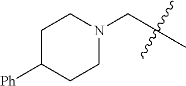 [SMILES: *CN1CCC(c2ccccc2)CC1] |
| S21-6-4 | 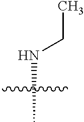 | 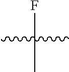 [SMILES: *F] | 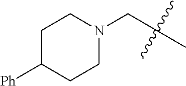 [SMILES: *CN1CCC(c2ccccc2)CC1] |
| S21-6-2 | 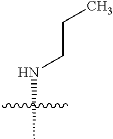 | 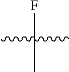 [SMILES: *F] | 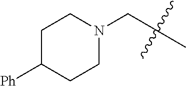 [SMILES: *CN1CCC(c2ccccc2)CC1] |
| S21-6-5 | 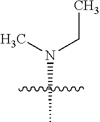 | 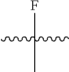 [SMILES: *F] | 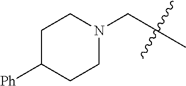 [SMILES: *CN1CCC(c2ccccc2)CC1] |
| S21-6-6 | 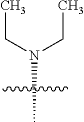 | 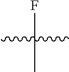 [SMILES: *F] | 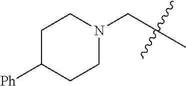 [SMILES: *CN1CCC(c2ccccc2)CC1] |
| S21-6-7 | 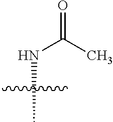 | 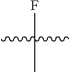 [SMILES: *F] | 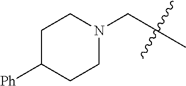 [SMILES: *CN1CCC(c2ccccc2)CC1] |
| S21-6-8 | 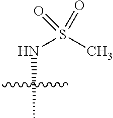 | 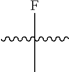 [SMILES: *F] | 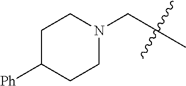 [SMILES: *CN1CCC(c2ccccc2)CC1] |

In the 38^{th} embodiment, the present invention is a method of treating a hematological cancer comprising administering to a subject in need of treatment an effective amount of a compound represented by any one of structural formulas:

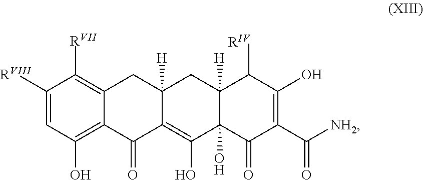

or a pharmaceutically acceptable salt thereof or a pharmaceutically acceptable composition thereof, wherein:

| Compound | | | |
|---|---|---|---|
| number | R^{IV} | R^{VII} | R^{VIII} |
| K1 | 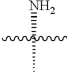 | 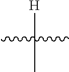 [SMILES: *[H]] | 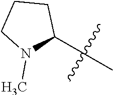 [SMILES: *[C@@H]1CCCN1C] |
| K2 | 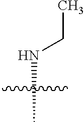 | 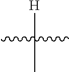 [SMILES: *[H]] | 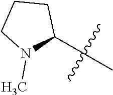 [SMILES: *[C@@H]1CCCN1C] |
| K3 | 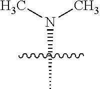 | 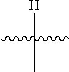 [SMILES: *[H]] | 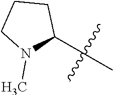 [SMILES: *[C@@H]1CCCN1C] |
| K4 (compound 3A) | 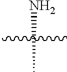 | 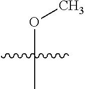 [SMILES: *OC] | 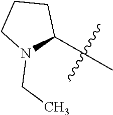 [SMILES: *[C@@H]1CCCN1CC] |
| K5 | 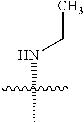 | 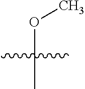 [SMILES: *OC] | 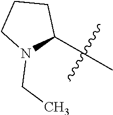 [SMILES: *[C@@H]1CCCN1CC] |
| K6 | 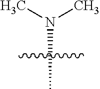 | 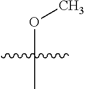 [SMILES: *OC] | 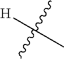 [SMILES: *[H]] |
| K7 | 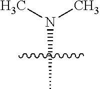 | 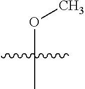 [SMILES: *OC] | 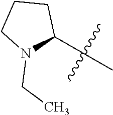 [SMILES: *[C@@H]1CCCN1CC] |
| K8 | 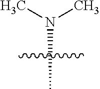 | 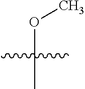 [SMILES: *OC] | 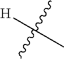 [SMILES: *[H]] |
| K9 | 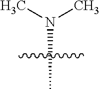 | 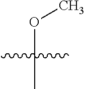 [SMILES: *OC] | 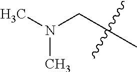 [SMILES: *CN(C)C] |
| K10 | 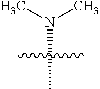 | 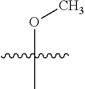 [SMILES: *OC] | 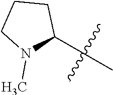 [SMILES: *[C@@H]1CCCN1C] |
| K11 | 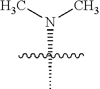 | 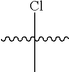 [SMILES: *Cl] | 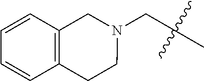 [SMILES: *CN1CCc2ccccc2C1] |
| K12 | 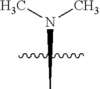 | 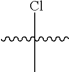 [SMILES: *Cl] | 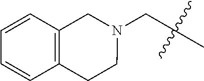 [SMILES: *CN1CCc2ccccc2C1] |
| K13 | 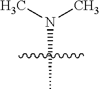 | 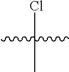 [SMILES: *Cl] | 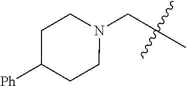 [SMILES: *CN1CCC(c2ccccc2)CC1] |
| K14 | 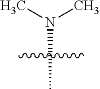 | 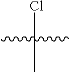 [SMILES: *Cl] | 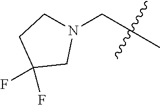 [SMILES: *CN1CCC(F)(F)C1] |
| K15 | 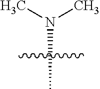 | 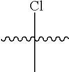 [SMILES: *Cl] | 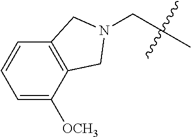 [SMILES: *CN1Cc2cccc(OC)c2C1] |
| K16 | 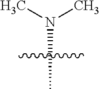 | 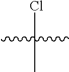 [SMILES: *Cl] | 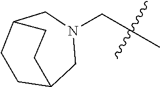 [SMILES: *CN1CC2CCC(CC2)C1] |
| K17 | 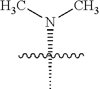 | 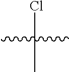 [SMILES: *Cl] | 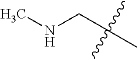 [SMILES: *CNC] |
| K18 | 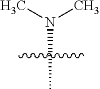 | 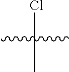 [SMILES: *Cl] | 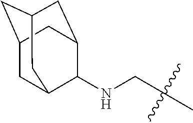 [SMILES: *CNC1C2CC3CC(C2)CC1C3] |
| K19 | 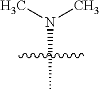 | 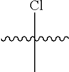 [SMILES: *Cl] | 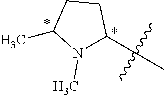 [SMILES: *C1CCC(C)N1C] |
| K20 | 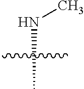 | 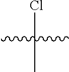 [SMILES: *Cl] | 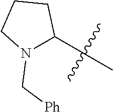 [SMILES: *C1CCCN1Cc1ccccc1] |
| K21 | 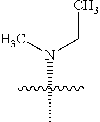 | 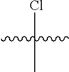 [SMILES: *Cl] | 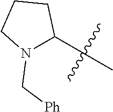 [SMILES: *C1CCCN1Cc1ccccc1] |
| K22 | 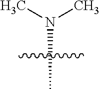 | 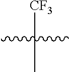 [SMILES: *C(F)(F)F] | 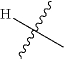 [SMILES: *[H]] |
| K23 | 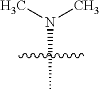 | 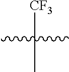 [SMILES: *C(F)(F)F] | 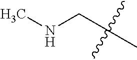 [SMILES: *CNC] |
| K24 | 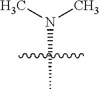 | 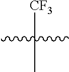 [SMILES: *C(F)(F)F] | 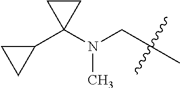 [SMILES: *CN(C)C1(C2CC2)CC1] |
| K25 | 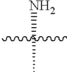 | 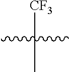 [SMILES: *C(F)(F)F] | 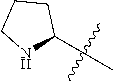 [SMILES: *[C@@H]1CCCN1] |
| K26 | 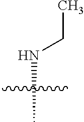 | 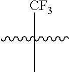 [SMILES: *C(F)(F)F] | 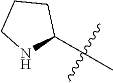 [SMILES: *[C@@H]1CCCN1] |
| K27 | 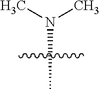 | 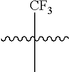 [SMILES: *C(F)(F)F] | 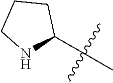 [SMILES: *[C@@H]1CCCN1] |
| K28 | 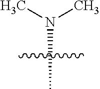 | 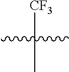 [SMILES: *C(F)(F)F] | 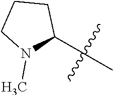 [SMILES: *[C@@H]1CCCN1C] |
| K29 | 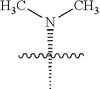 | 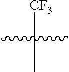 [SMILES: *C(F)(F)F] | 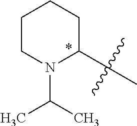 [SMILES: *C1CCCCN1C(C)C] |
| K30 | 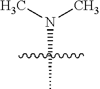 | 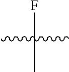 [SMILES: *F] | 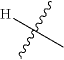 [SMILES: *[H]] |
| K31 | 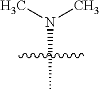 | 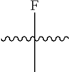 [SMILES: *F] | 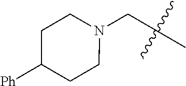 [SMILES: *CN1CCC(c2ccccc2)CC1] |
| K32 | 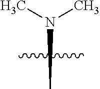 | 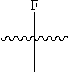 [SMILES: *F] | 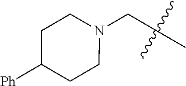 [SMILES: *CN1CCC(c2ccccc2)CC1] |
| K33 | 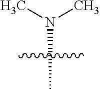 | 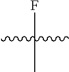 [SMILES: *F] | 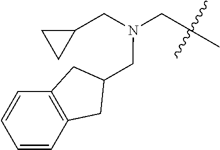 [SMILES: *CN(CC1CC1)CC1Cc2ccccc2C1] |
| K34 | 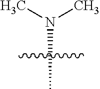 | 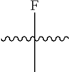 [SMILES: *F] | 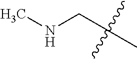 [SMILES: *CNC] |
| K35 | 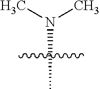 | 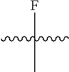 [SMILES: *F] | 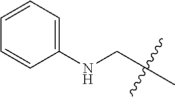 [SMILES: *CNc1ccccc1] |
| K36 | 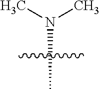 | 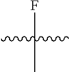 [SMILES: *F] | 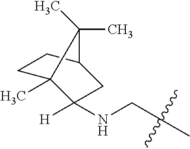 [SMILES: *CNC1CC2CCC1(C)C2(C)C] |
| K37 | 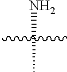 | 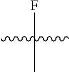 [SMILES: *F] | 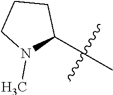 [SMILES: *[C@@H]1CCCN1C] |
| K38 | 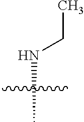 | 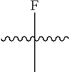 [SMILES: *F] | 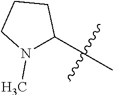 [SMILES: *C1CCCN1C] |
| K39 | 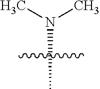 | 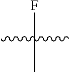 [SMILES: *F] | 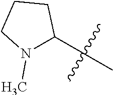 [SMILES: *C1CCCN1C] |
| K40 | 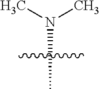 | 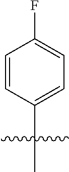 [SMILES: *c1ccc(F)cc1] | 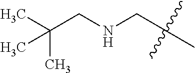 [SMILES: *CNCC(C)(C)C] |
| K41 | 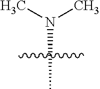 | 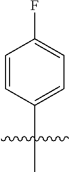 [SMILES: *c1ccc(F)cc1] | 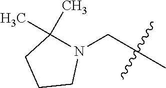 [SMILES: *CN1CCCC1(C)C] |
| K42 | 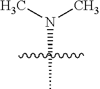 | 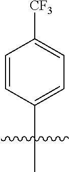 [SMILES: *c1ccc(C(F)(F)F)cc1] | 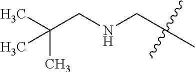 [SMILES: *CNCC(C)(C)C] |

In a 40^{th} embodiment, the present invention is a compound represented by any one of structural formulas (XIV) or (XV):

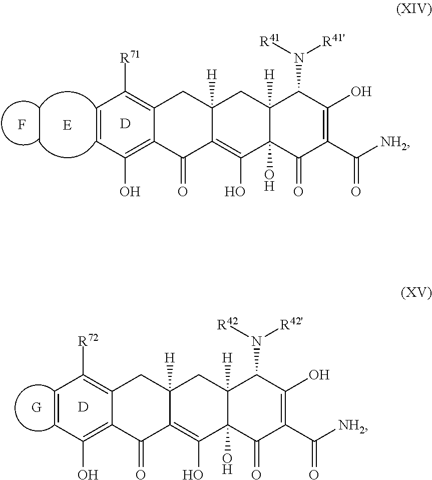

or a pharmaceutically acceptable salt thereof. In a first aspect of the 40^{th} embodiment, ring E is a 4- or 5-member carbocyclyl; ring F is a 5- or 6-member heterocyclyl that includes at least one nitrogen atom; ring G is represented by any one of the following structural formulas

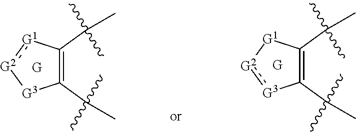

wherein “ [symbol] ” represents the point of attachment of ring G to ring D, “ [symbol] ” is a single or a double bond, G¹, G², and G³, each independently, is —CH═, —CH₂—, —N═, or —NH—, as valence permits, provided that when “ [symbol] ” is a single bond, then at least two of G¹, G², and G³ are —NH—;

R⁷¹ and R⁷², each independently, is selected from hydrogen, halo, —(C₁-C₆ alkyl), —OR^{A}, —C(O)NR^{B}R^{B′}, NR^{B}R^{B′}, S(O)₀₋₂R^{C}, —(C₀-C₆ alkylenyl)-(C₃₋₁₂)carbocyclyl, and —(C₀-C₆ alkylenyl)-(4- to 13-member)heterocyclyl;

R⁴¹, R^{41′}, R⁴², and R^{42′}, each independently, is selected from hydrogen, —(C₁-C₆ alkyl), S(O)₁₋₂R^{C}, —(C₀-C₆ alkylenyl)-(C₃₋₁₂)carbocyclyl, —(C₀-C₆ alkylenyl)-(4- to 13-member)heterocyclyl, —C(O)—(C₁-C₆ alkyl), and —C(O)—(C₁-C₆ alkyl)-NR^{D}R^{E}; or

R⁴¹ and R^{41′}, and, separately, R⁴² and R^{42′}, are taken together with the nitrogen atom to which they are commonly bound to form a 4-8 membered ring optionally comprising 1-2 additional heteroatoms independently selected from N, O and S;

each R^{A} is independently selected from hydrogen, —(C₁-C₆ alkyl), —(C₀-C₆ alkylenyl)-(C₃₋₁₂)carbocyclyl, —(C₀-C₆ alkylenyl)-(4- to 13-member)heterocyclyl, —C(O)—(C₁-C₆ alkyl), —C(O)—(C₀-C₆ alkylenyl)-(C₃₋₁₂)carbocyclyl, —C(O)—(C₀-C₆ alkylenyl)-(4- to 13-member)heterocyclyl, and —C(O)N(R^{D})(R^{E});

each R^{B} and each R^{B′} is independently selected from hydrogen, —(C₁-C₆ alkyl), —(C₁-C₆ haloalkyl), —(C₀-C₆ alkylenyl)-(C₃₋₁₂)carbocyclyl, —(C₀-C₆ alkylenyl)-(4- to 13-member)heterocyclyl, —S(O)₁₋₂—(C₁-C₆ alkyl), —S(O)₁₋₂—(C₀-C₆ alkylenyl)-(C₃₋₁₂)carbocyclyl, —S(O)₁₋₂—(C₀-C₆ alkylenyl)-(4- to 13-member)heterocyclyl, —C(O)—(C₁-C₆ alkyl), —C(O)—(C₀-C₆ alkylenyl)-(C₃₋₁₂)carbocyclyl, —C(O)H, —C(O)—(C₀-C₆ alkylenyl)-(4- to 13-member)heterocyclyl, and —C(O)—(C₀-C₆ alkylenyl)-N(R^{D})(R^{E});

each R^{C} is independently selected from —(C₁-C₆ alkyl), —(C₀-C₆ alkylenyl)-(C₃₋₁₂)carbocyclyl and —(C₀-C₆ alkylenyl)-(4- to 13-member)heterocyclyl; and

each R^{D} and each R^{E} is independently selected from hydrogen, —(C₁-C₆ alkyl), —(C₀-C₆ alkylenyl)-(C₃₋₁₂)carbocyclyl, and —(C₀-C₆ alkylenyl)-(4- to 13-member)heterocyclyl;

wherein:

any alkyl, or alkylenyl portion of R⁷¹, R⁷², R⁴¹, R^{41′}, R⁴², or R^{42′} is optionally and independently substituted with one or more substituents independently selected from halo, ═O, OR^{A}, NR^{B}R^{B′}, and S(O)₀₋₂R^{C};

any alkyl or alkylenyl portion of R^{A} or R^{C}, is optionally and independently substituted with one or more fluoro;

rings E, F, and G, or any carbocyclyl or heterocyclyl portion of any of R⁷¹, R⁷², R⁴¹, R^{41′}, R⁴², or R^{42′}, or any ring formed by taking together R⁴¹ and R^{41′} or R⁴² and R^{42′} is optionally and independently substituted on a carbon atom with one or more substituents independently selected from halo, ═O, C₁-C₄ fluoroalkyl, C₁-C₄ alkyl, —(C₀-C₆ alkylenyl)-(C₃₋₁₂ carbocyclyl), —(C₀-C₆ alkylenyl)-(4- to 13-membered heterocyclyl), OR^{A}, —(C₀-C₆ alkylenyl)-NR^{B}R^{B′}, and S(O)₀₋₂R^{C};

rings F and G, or any heterocyclyl portion of any of R⁷¹, R⁷², R⁴¹, R^{41′}, R⁴², or R^{42′}, or any ring formed by taking together R⁴¹ and R^{41′} or R⁴² and R^{42′} is optionally and independently substituted on a substitutable nitrogen atom with R^{F};

each R^{F} is independently selected from —(C₁-C₆ alkyl), —(C₁-C₆ haloalkyl), —(C₁-C₆ hydroxyalkyl), —(C₀-C₆ alkylenyl)-(C₃₋₁₂)carbocyclyl, —(C₀-C₆ alkylenyl)-(4- to 13-member)heterocyclyl, —S(O)₁₋₂—(C₁-C₆ alkyl), —S(O)₁₋₂—(C₀-C₆ alkylenyl)-(C₃₋₁₂)carbocyclyl, —S(O)₁₋₂—(C₀-C₆ alkylenyl)-(4- to 13-member)heterocyclyl, —C(O)—(C₁-C₆ alkyl), —C(O)—(C₀-C₆ alkylenyl)-(C₃₋₁₂)carbocyclyl, —C(O)H, —C(O)—(C₀-C₆ alkylenyl)-(4- to 13-member)heterocyclyl, —(C₀-C₆ alkylenyl)-C(O)₂—(C₁-C₆ alkyl), —(C₁-C₆ alkylenyl)-NR^{B}R^{B′} and —C(O)N(R^{D})(R^{E});

any carbocyclyl or heterocyclyl portion of R^{A}, R^{B}, R^{B′}, R^{C}, R^{D}, R^{E}, R^{F}, or any substituent of R⁷¹, R⁷², R⁴¹, R^{41′}, R⁴², or R^{42′} is optionally and independently substituted on a carbon atom with one or more substituents independently selected from fluoro, chloro, C₁-C₄ alkyl, C₁-C₄ fluoroalkyl, —O—C₁-C₄ alkyl, —O—C₁-C₄ fluoroalkyl, ═O, —OH, —NH₂, —NH(C₁-C₄ alkyl), and —N(C₁-C₄ alkyl)₂; and

any heterocyclyl portion of R^{A}, R^{B}, R^{B′}, R^{C}, R^{D}, R^{E}, R^{F}, or any heterocyclyl substituent of R⁷¹, R⁷², R⁴¹, R^{41′}, R⁴², or R^{42′} is optionally substituted on a substitutable nitrogen atom with —C₁-C₄ alkyl, or —S(O)₁₋₂—(C₁-C₄ alkyl).

In the second aspect of the 40^{th} embodiment, ring E and ring F, together, are represented by any one of the following structural formulas:

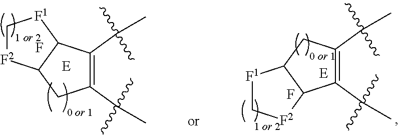

wherein F¹ and F², for each occurrence independently, is selected from —CH₂— or —NR⁰—, wherein R⁰, for each occurrence independently, is H or a C₁-C₄ alkyl, and “ [symbol] ” represents the point of attachment of ring E to ring D. The remainder of the values and example values of the variables in structural formulas (XIV) and (XV) of the 40^{th} embodiment are as defined above with respect to aspect one of the 40^{th} embodiment.

In the third aspect of the 40^{th} embodiment, R⁴¹, R^{41′}, R⁴², or R^{42′}, each independently, is selected from hydrogen; —(C₁-C₆ alkyl), optionally substituted with one or more substituents independently selected from hydroxy and halo; —(C₃-C₆ cycloalkyl); —C(O)—(C₁-C₆ alkyl); —C(O)—(C₁-C₆ alkylenyl)-N(R^{D})(R^{E}); and S(O)₁₋₂R^{C}; or R⁴¹ and R^{41′} or R⁴² and R^{42′} are taken together with the nitrogen atom to which they are commonly bound to form a 4-6 membered ring optionally comprising 1-2 additional heteroatoms independently selected from N, O and S; R^{C} is —(C₁-C₆ alkyl); and each of R^{D} and R^{E} is independently selected from hydrogen and —(C₁-C₆ alkyl). The remainder of the values and example values of the variables in structural formulas (XIV) and (XV) of the 40^{th} embodiment are as defined above with respect to aspects one and two of the 40^{th} embodiment.

In the fourth aspect of the 40^{th} embodiment, R⁴¹, R^{41′}, R⁴², or R^{42′}, each independently, is selected from hydrogen, —(C₁-C₆ alkyl), —(C₃-C₆ cycloalkyl), —C(O)—(C₁-C₆ alkyl), —C(O)—(C₁-C₆ alkylenyl)-N(R^{D})(R^{E}), and S(O)₁₋₂R^{C}; R^{C} is —(C₁-C₆ alkyl); and each of R^{D} and R^{E} is independently selected from hydrogen and —(C₁-C₆ alkyl). The remainder of the values and example values of the variables in structural formulas (XIV) and (XV) of the 40^{th} embodiment are as defined above with respect to aspects one through three of the 40^{th} embodiment.

In the fifth aspect of the 40^{th} embodiment, R⁴¹, R^{41′}, R⁴², or R^{42′}, each independently, is selected from hydrogen, methyl, ethyl, propyl, cyclopropyl, —C(O)CH₃, —C(O)CH₂N(CH₃)₂, and —S(O)₂CH₃. The remainder of the values and example values of the variables in structural formulas (XIV) and (XV) of the 40^{th} embodiment are as defined above with respect to aspects one through four of the 40^{th} embodiment.

In the sixth aspect of the 40^{th} embodiment, R⁷¹ and R⁷², each independently, is selected from hydrogen; halo; —(C₁-C₆ alkyl), optionally substituted with one or more substituents independently selected from hydroxyl, halo,

and —NR^{B}R^{B′}; —NR^{B}R^{B′}; —C(O)NR^{B}R^{B′}, —OR^{A}, —(C₀-C₆ alkylenyl)-(C₃-C₈)carbocyclyl, and —(C₀-C₆ alkylenyl)-(4- to 8-member)heterocyclyl, wherein R^{A} is C₁-C₆ alkyl optionally substituted with one or more fluoro. For example, R⁷¹ and R⁷², each independently, is selected from hydrogen; halo; —(C₁-C₆ alkyl), optionally substituted with one or more halo; and —OR^{A}, wherein R^{A} is C₁-C₆ alkyl optionally substituted with one or more fluoro. The remainder of the values and example values of the variables in structural formulas (XIV) and (XV) of the 40^{th} embodiment are as defined above with respect to aspects one through five of the 40^{th} embodiment.

In the seventh aspect of the 40^{th} embodiment, R⁷¹ and R⁷², each independently, is selected from hydrogen, fluoro, chloro, —CF₃, —OCH₃, —OCF₃, —N(CH₃)₂ and —NHCH₃. The remainder of the values and example values of the variables in structural formulas (XIV) and (XV) of the 40^{th} embodiment are as defined above with respect to aspects one through seven of the 40^{th} embodiment.

In the eight aspect of the 40^{th} embodiment, ring E is represented by the following structural formula

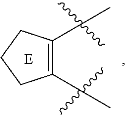 [SMILES: *C1=C(*)CCC1]

wherein each “ [symbol] ” represents a point of attachment of the ring E to the ring D. The remainder of the values and example values of the variables in structural formulas (XIV) and (XV) of the 40^{th} embodiment are as defined above with respect to aspects one through seven of the 40^{th} embodiment.

In the ninth aspect of the 40^{th} embodiment, wherein ring E is represented by the following structural formula

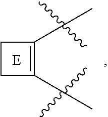 [SMILES: *C1=C(*)CC1]

wherein each “ [symbol] ” represents a point of attachment of the ring E to the ring D. The remainder of the values and example values of the variables in structural formulas (XIV) and (XV) of the 40^{th} embodiment are as defined above with respect to aspects one through eight of the 40^{th} embodiment.

In the tenth aspect of the 40^{th} embodiment, ring F is represented by any one of the following structural formulas

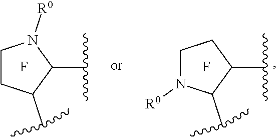

wherein each “ [symbol] ” represents a point of attachment of the ring F to the ring E, and wherein R⁰,
for each occurrence independently, is H or a C1-C4 alkyl. The remainder of the values and example values of the variables in structural formulas (XIV) and (XV) of the 40th embodiment are as defined above with respect to aspects one through nine of the 40th embodiment.

In the eleventh aspect of the 40^{th} embodiment, ring G is represented by any one of the following structural formulas:

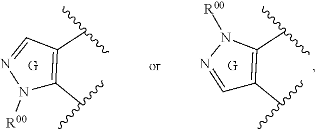

wherein each “ [symbol] ” represents a point of attachment of the ring G to the ring D, and wherein R⁰⁰, for each occurrence independently, is H or a C1-C4 alkyl. The remainder of the values and example values of the variables in structural formulas (XIV) and (XV) of the 40th embodiment are as defined above with respect to aspects one through ten of the 40th embodiment.

In the twelfth aspect of the 40^{th} embodiment, R⁴¹, R^{41′}, R⁴² or R^{42′}, each independently, is H or a C1-C4 alkyl; R⁷¹ and R⁷², each independently, is F or —CF₃. The remainder of the values and example values of the variables in structural formulas (XIV) and (XV) of the 40^{th} embodiment are as defined above with respect to aspects one through eleven of the 40^{th} embodiment.

In the thirteenth aspect of the 40^{th} embodiment, ring E is represented by the following structural formula

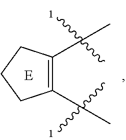 [SMILES: *C1=C(*)CCC1]

wherein each “1 [symbol] ” represents a point of attachment or the ring E to the ring D, ring F is represented by any one of the following structural formulas

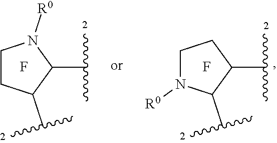

wherein each “2 [symbol] ” represents a point of attachment or the ring F to the ring E, R⁰, for each occurrence independently, is H or a C1-C4 alkyl; R⁴¹, R^{41′}, R⁴², or R^{42′}, each independently, is H or a C1-C4 alkyl; and R⁷¹ and R⁷², each independently, is F or —CF₃. The remainder of the values and example values of the variables in structural formulas (XIV) and (XV) of the 40^{th} embodiment are as defined above with respect to aspects one through twelve of the 40^{th} embodiment.

In the fourteenth aspect of the 40^{th} embodiment, ring E is represented by the following structural formula

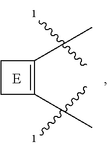 [SMILES: *C1=C(*)CC1]

wherein each “1 [symbol] ” represents a point of attachment of the ring E to the ring D, ring F is represented by any one of the following structural formulas

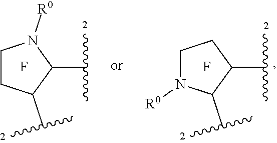

wherein each “2 [symbol] ” represents a point of attachment of the ring F to the ring E, R⁰, for each occurrence independently, is H or a C₁-C₄ alkyl; R⁴¹, R^{41′}, R⁴², or R^{42′}, each independently, is H or a C₁-C₄ alkyl; and R⁷¹ and R⁷², each independently, is F or —CF₃. The remainder of the values and example values of the variables in structural formulas (XIV) and (XV) of the 40th embodiment are as defined above with respect to aspects one through thirteen of the 40th embodiment.

In the fifteenth aspect of the 40^{th} embodiment, ring G is represented by any one of the following structural formulas:

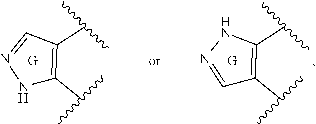 [SMILES: *c1cn[nH]c1* ; *c1cn[nH]c1*]

wherein each “ [symbol] ” represents a point of attachment of the ring G to the ring D; R⁴¹, R^{41′}, R⁴², or
   R^{42′}, each independently, is H or a C₁-C₄ alkyl; and R⁷¹ and R⁷², each independently, is F or —CF₃. The remainder of the values and example values of the variables in structural formulas (XIV) and (XV) of the 40^{th} embodiment are as defined above with respect to aspects one through fourteen of the 40^{th} embodiment.

In the sixteenth aspect of the 40^{th} embodiment, the compound is represented by any one of the following structural formulas, or a pharmaceutically acceptable salt thereof:

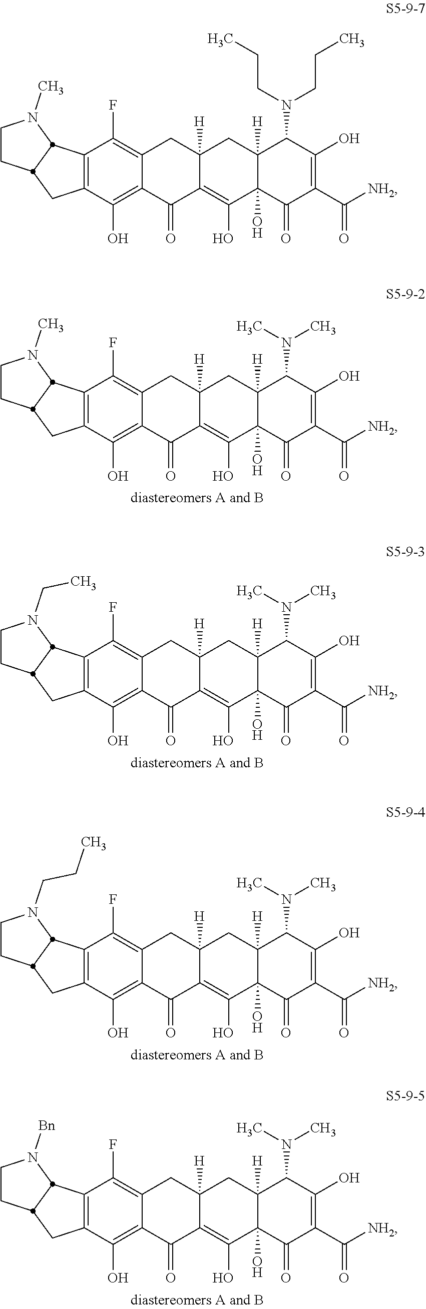

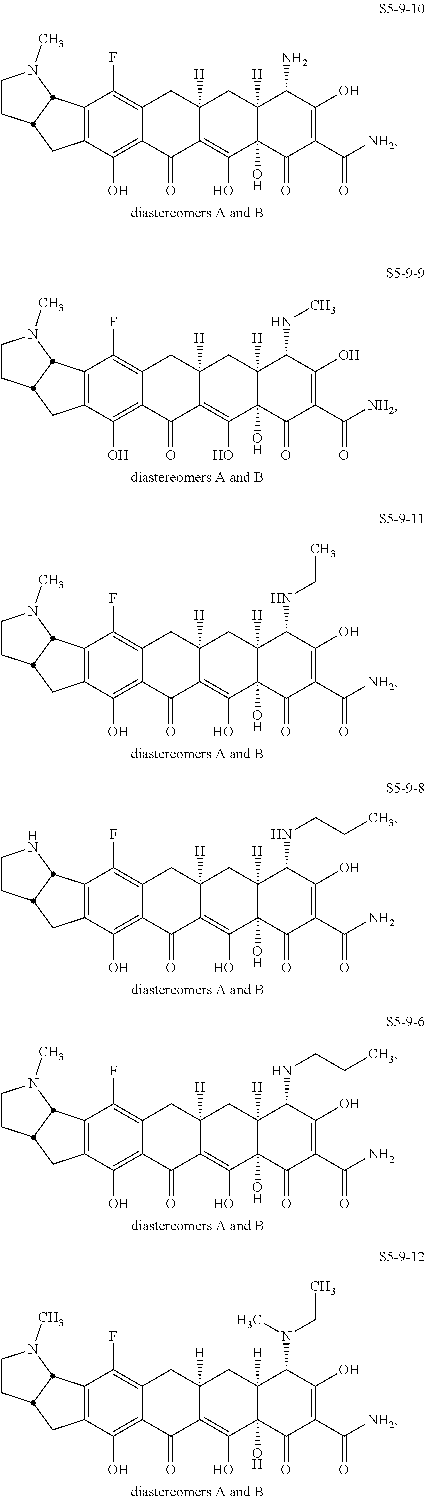

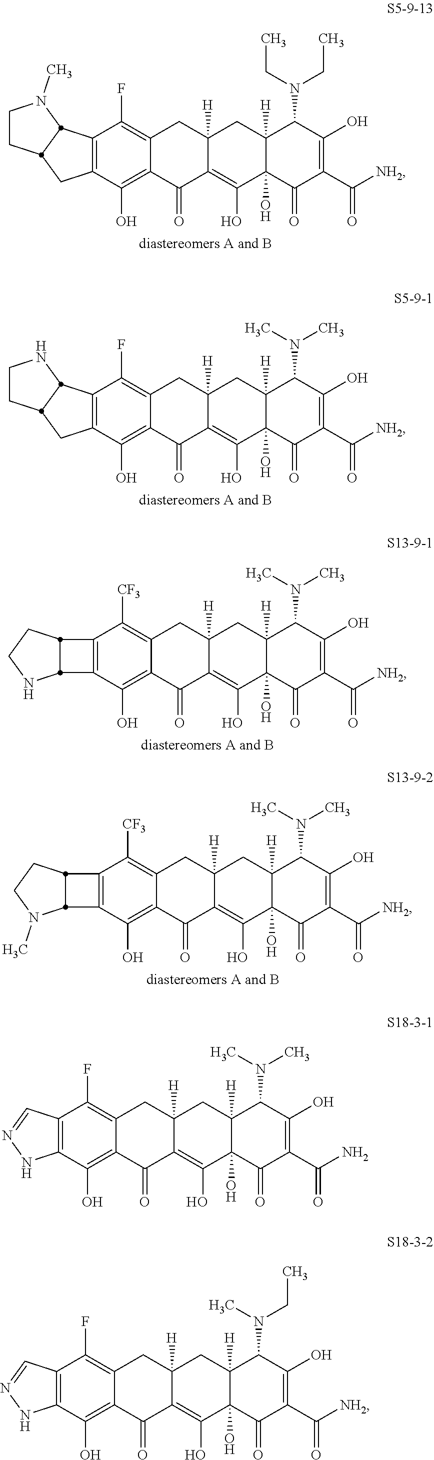

or a pharmaceutically acceptable salt of any of the foregoing.

In the seventeenth aspect of the 40^{th} embodiment, the compound is represented by the following structural formula

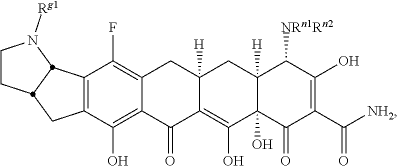

or a pharmaceutically acceptable salt thereof, wherein R^{g1}, Rⁿ¹, and Rⁿ², each independently, is H
   or a C₁-C₄ alkyl, optionally substituted with a phenyl. The remainder of the values and example values of the variables in structural formulas (XIV) and (XV) of the 40th embodiment are as defined above with respect to aspects one through fifteen of the 40th embodiment.

In the eighteenth aspect of the 40^{th} embodiment, the compound is represented by any one of the following structural formulas:

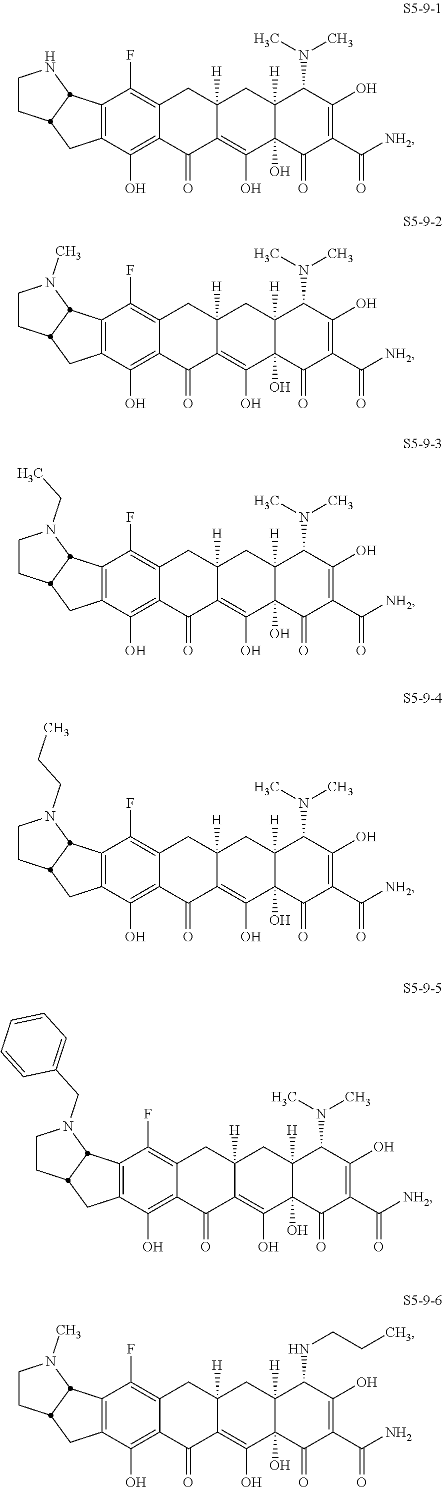

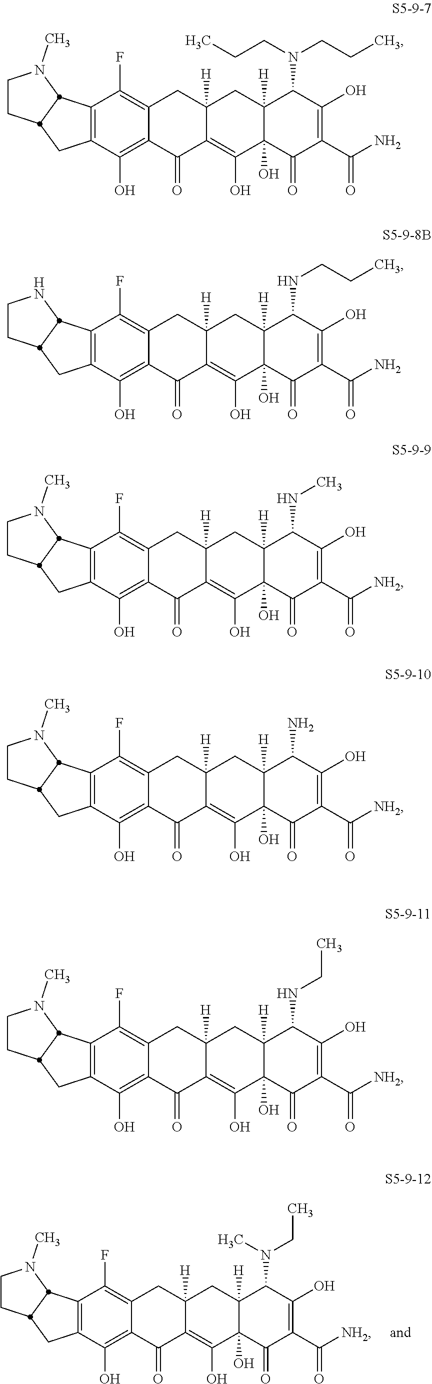

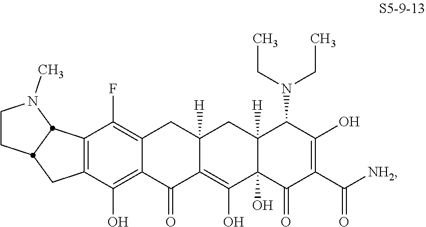 [SMILES: CCN(CC)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

or a pharmaceutically acceptable salt of any of the foregoing.

In the nineteenth aspect of the 40^{th} embodiment, the compound is represented by the following structural formula

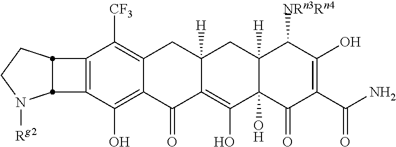

or a pharmaceutically acceptable salt thereof, wherein R^{g2}, Rⁿ³, and Rⁿ⁴, each independently, is H or a C₁-C₄ alkyl. The remainder of the values and example values of the variables in structural formulas (XIV) and (XV) of the 40^{th} embodiment are as defined above with respect to aspects one through fifteen of the 40^{th} embodiment.

In the twentieth aspect of the 40^{th} embodiment, the compound is represented by any one of the following structural formulas:

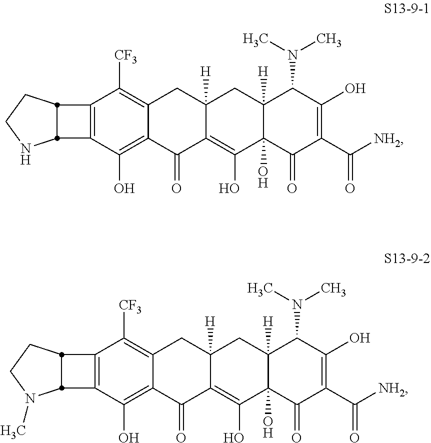 [SMILES: CN(C)[C@@H]1C(O)=C(C(N)=O)C(=O)[C@@]2(O)C(O)=C3C(=O)c4c(O)c5c(c(C(F)(F)F)c4C[C@H]3C[C@@H]12)[C@H]1CCN[C@@H]51 ; CN(C)[C@@H]1C(O)=C(C(N)=O)C(=O)[C@@]2(O)C(O)=C3C(=O)c4c(O)c5c(c(C(F)(F)F)c4C[C@H]3C[C@@H]12)[C@H]1CCN(C)[C@@H]51]

or a pharmaceutically acceptable salt thereof.

In the twenty-first aspect of the 40^{th} embodiment, the compound is represented by the following structural formula

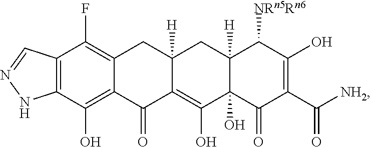

or a pharmaceutically acceptable salt thereof, wherein Rⁿ⁵ and Rⁿ⁶, each independently, is H or a
   C₁-C₄ alkyl. The remainder of the values and example values of the variables in structural formulas (XIV) and (XV) of the 40^{th} embodiment are as defined above with respect to aspects one through fifteen of the 40^{th} embodiment.

In the twenty-second aspect of the 40^{th} embodiment, the compound is represented by any one of the following structural formulas:

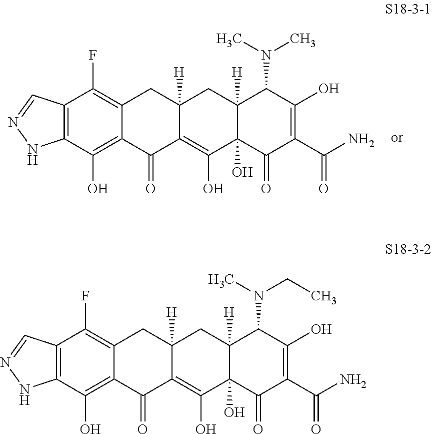 [SMILES: CN(C)[C@@H]1C(O)=C(C(N)=O)C(=O)[C@@]2(O)C(O)=C3C(=O)c4c(c(F)c5cn[nH]c5c4O)C[C@H]3C[C@@H]12 ; "or" ; CCN(C)[C@@H]1C(O)=C(C(N)=O)C(=O)[C@@]2(O)C(O)=C3C(=O)c4c(c(F)c5cn[nH]c5c4O)C[C@H]3C[C@@H]12]

or a pharmaceutically acceptable salt of any of the foregoing.

In the 41^{st} embodiment, the present invention is a pharmaceutical composition comprising a pharmaceutically acceptable carrier or diluent and a compound of any compound described herein with respect to embodiments 1 through 40, in particular embodiments 37-40, and various aspects thereof.

In the 42^{nd} embodiment, the present invention is a method of treating a subject suffering from a hematological tumor, comprising administering to the subject a therapeutically effective amount of any compound described herein with respect to embodiments 1 through 40 and various aspects thereof, or a pharmaceutically acceptable salt thereof, or a pharmaceutical composition of embodiment 41.

In the first aspect of the 42^{nd} embodiment, the hematological cancer is a leukemia.

Examples of leukemia include acute myeloid leukemia, acute lymphoblastic leukemia, chronic lymphocytic leukemia, chronic myelogenous leukemia, chronic myelomonocytic leukemia, acute monocytic leukemia.

In the second aspect of the 42^{nd} embodiment, the leukemia is acute myeloid leukemia.

In the third aspect of the 42^{nd} embodiment, the hematological cancer is a lymphoma. Examples of lymphomas include Hodgkin's lymphoma, non-Hodgkin's lymphomas, multiple myeloma, myelodysplastic or myeloproliferative syndrome, mantle cell lymphoma, diffuse large B-cell lymphoma (DLBCL), Burkitt lymphoma/leukemia and B-cell lymphoma.

In the fourth aspect of the 42^{nd} embodiment, the method includes administration of one or more additional therapeutic agents. Examples of the additional therapeutic agents include cytarabine and an anthracycline drugs. Examples of the anthracycline drug include daunorubicin or idarubicin.

In the fifth aspect of the 42^{nd} embodiment, the method further includes administration of cladribine.

In various aspects of the 42^{nd} embodiment, the subject is a human.

In the 43^{rd} embodiment, the present invention is a method for treating a bacterial infection in a subject (including preventing an infection or colonization in a subject) in need thereof, comprising administering to the subject a therapeutically effective amount of any compound described herein with respect to embodiments 1 through 40, particularly embodiments 37-40, and various aspects thereof, or a pharmaceutically acceptable salt thereof, or a pharmaceutical composition of embodiment 41.

In the first aspect of the 43^{rd} embodiment, the infection is caused by a Gram-positive organism. Examples of the Gram-positive organisms include an organism selected from the class Bacilli; phylum Actinobacteria; and class Clostridia.

In the second aspect of the 43^{rd} embodiment, the infection is caused by a Gram-negative organism. Examples of Gram-negative organisms include an organism selected from the group consisting of Enterobactericeae, Bacteroidetes, Vibrionaceae, Pasteurellaceae, Pseudomonadaceae, Neisseriaceae, Rickettsiae, Moraxellaceae any species of Proteeae, Acinetobacter spp., Helicobacter spp., and Campylobacter spp.

In the third aspect of the 43^{rd} embodiment, the infection is caused by an organism selected from order Rickettsiales and order Chlamydiales.

In the fourth aspect of the 43^{rd} embodiment, the infection is caused by an organism selected from the phylum Chlamydiae and phylum Spriochaetales.

In the fifth aspect of the 43^{rd} embodiment, the infection is caused by an organism selected from the class Mollicutes.

In the sixth aspect of the 43^{rd} embodiment, the infection is caused by more than one organism.

In the seventh aspect of the 43^{rd} embodiment, the infection is caused by an organism resistant to one or more antibiotics.

In the eighth aspect of the 43^{rd} embodiment, the infection is caused by a Gram-positive organism, and the Gram-positive organism is selected from S. aureus, CoNS, S. pneumoniae, S. pyogenes, S. agalactiae, E. faecalis and E. faecium.

In the ninth aspect of the 43^{rd} embodiment, the infection is caused by a Gram-negative organism, and the Gram-negative organism is selected from H. influenza, M. catarrhalis and Legionella pneumophila.

### Definitions

“Alkyl” means an optionally substituted saturated aliphatic branched or straight-chain monovalent hydrocarbon radical having the specified number of carbon atoms. Thus, “(C₁-C₆) alkyl” means a radical having from 1-6 carbon atoms in a linear or branched arrangement. “(C₁-C₆)alkyl” includes methyl, ethyl, propyl, butyl, pentyl and hexyl. “(C₁-C₁₂) alkyl” means a radical having from 1-12 carbon atoms in a linear or branched arrangement. “(C₁-C₁₂)alkyl” includes methyl, ethyl, propyl, butyl, pentyl, hexyl, heptyl, octyl, nonyl, decyl, undecyl and dodecyl. Unless otherwise specified, suitable substitutions for a “substituted alkyl” include halogen, —OH, —O—C₁-C₄ alkyl, C₁-C₄ alkyl, fluoro-substituted-C₁-C₄ alkyl, —O—C₁-C₄ fluoroalkyl, —NH₂, —NH(C₁-C₄ alkyl), —N(C₁-C₄ alkyl)₂, C₃-C₁₂ carbocyclyl (e.g., cyclopropyl, cyclobutyl, cyclopentyl, cyclohexyl, phenyl or naphthalenyl), a (4-13 membered) heterocyclyl (e.g., pyrrolidine, piperidine, piperazine, tetrahydrofuran, tetrahydropyran or morpholine) or —N(R^{X})(R^{X′}), wherein R^{X} and R^{X′} are independently hydrogen or C₁-C₄ alkyl, or taken together with the nitrogen atom to which they are bound form a (4-7 membered) heterocylic ring optionally comprising one additional heteroatom selected from N, S and O, wherein the (4-7 membered) heterocylic ring is optionally substituted with fluoro, chloro, —OH, fluoro-substituted C₁-C₄ alkyl, —C₁-C₄ alkyl, or —C₀-C₄ alkylene-O—C₁-C₄ alkyl, and is optionally benzofused.

“Benzofused,” when referring to a ring system, means fused to a phenyl ring, forming a fused bicyclyl ring.

“Alkylene” or “alkylenyl” (used interchangeably) mean an optionally substituted saturated aliphatic branched or straight-chain divalent hydrocarbon radical having the specified number of carbon atoms. An alkyl moiety of an alkylene group can be a part of a larger moiety such as alkoxy, alkylammonium, and the like. Thus, “(C₁-C₆)alkylene” means a divalent saturated aliphatic radical having from 1-6 carbon atoms in a linear arrangement, e.g., —[(CH₂)ₙ]—, where n is an integer from 1 to 6, “(C₁-C₆)alkylene” includes methylene, ethylene, propylene, butylene, pentylene and hexylene. Alternatively, “(C₁-C₆)alkylene” means a divalent saturated radical having from 1-6 carbon atoms in a branched arrangement, for example: —[(CH₂CH₂CH₂CH₂CH(CH₃)]—, —[(CH₂CH₂CH₂CH₂C(CH₃)₂]—, —[(CH₂C(CH₃)₂CH (CH₃))]—, and the like. A “(C₁-C₁₂)alkylene” includes methyl, ethyl, n-propyl, iso-propyl, n-butyl, sec-butyl, tert-butyl, pentyl, hexyl, heptyl or octyl. A specific branched C₃-alkylene is

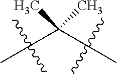

and a specific C₄-alkylene is

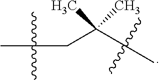

Other examples of a divalent C₁₋₆ alkyl group include, for example, a methylene group, an ethylene group, an ethylidene group, an n-propylene group, an isopropylene group, an isobutylene group, an s-butylene group, an n-butylene group, and a t-butylene group.

A “C₀ alkylenyl” is a covalent bond.

“Alkoxy” means an alkyl radical attached through an oxygen linking atom. “(C₁-C₄)-alkoxy” includes methoxy, ethoxy, propoxy, and butoxy.

“Alkylthio” means an alkyl radical attached through a sulfur linking atom. “(C₁-C₄)alkylthio” include methylthio, ethylthio, propylthio and butylthio.

“Alkylsulfinyl” means an alkyl radical attached through a —S(O)— linking group. “(C₁-C₄)alkylsulfinyl” include methylsulfinyl, ethylsulfinyl, propylsulfinyl and butylsulfinyl.

“Alkylsulfonyl” means an alkyl radical attached through a —S(O)₂— linking group. “(C₁-C₄)alkylsulfonyl” include methylsulfonyl, ethylsulfonyl, propylsulfonyl and butylsulfonyl.

“Aryl” or “aromatic” means an aromatic 6-18 membered monocyclic or polycyclic (e.g. bicyclic or tricyclic) carbocyclic ring system. In one embodiment, “aryl” is a 6-18 membered monocylic or bicyclic system. Aryl systems include, but not limited to, phenyl, naphthalenyl, fluorenyl, indenyl, azulenyl, and anthracenyl.

“Aryloxy” means an aryl moiety attached through an oxygen linking atom. Aryloxy includes, but not limited to, phenoxy.

“Arylthio” means an aryl moiety attached through a sulfur linking atom. Arylthio includes, but not limited to, phenylthio.

“Arylsulfinyl” means an aryl moiety attached through a —S(O)— linking group. Arylsulfinyl includes, but not limited to, phenylsulfinyl.

“Arylsulfonyl” means an aryl moiety attached through a —S(O)₂— linking group. Arylsulfonyl includes, but not limited to, phenylsulfonyl.

“Amine” means H₂N- and can also be used to refer to aminium group H₃N⁺—.

The term “alkylamine” includes a mono-, a dialkylamine and can also be used to refer to aminium (bearing a positive charge). A “monoalkyl amine” means an H(alkyl)N—, a “dialkylamine” means (alkyl)(alkyl)N—, and an “aminium” means (alkyl)(alkyl)(alkyl)N⁺—, H(alkyl)(alkyl)N⁺—, or H₂(alkyl)N⁺—, where each instance of “alkyl” independently refers to an alkyl having a specified number of atoms.

“Carbocyclyl” means a cyclic group having a specified number of atoms, wherein all ring atoms in the ring bound to the rest of the compound (also known as the “first ring”) are carbon atoms. Examples of “carbocyclyl” includes 3-18 (for example 3, 4, 5, 6, 7, 8, 9, 10, 11, 12, 12, 1, 14, 15, 16, 17, or 17 or any range therein, such as 3-12 or 3-10) membered saturated or unsaturated aliphatic cyclic hydrocarbon rings, or 6-18 membered aryl rings. A carbocyclyl moiety can be monocyclic, fused bicyclic, bridged bicyclic, spiro bicyclic, or polycyclic.

A “cycloalkyl” is an example of a fully saturated carbocyclyl.

Monocyclic carbocyclyls are saturated or unsaturated aliphatic cyclic hydrocarbon rings or aromatic hydrocarbon rings having the specified number of carbon atoms, such as 3-7 carbon atoms. Monocyclic carbocyclyls include cyclopropyl, cyclobutyl, cyclopentyl, cyclohexyl and cycloheptyl, cycloalkenyl, cycloalkynyl and phenyl.

A fused bicyclic carbocyclyl has two rings which have two adjacent ring atoms in common and can be, e.g., a (6-13 membered) fused bicyclic. The first ring attached to the parent molecular group is a monocyclic carbocyclyl and the ring fused to the first ring (also known as the “second ring”) is also a monocyclic carbocyclyl.

A bridged bicyclic carbocyclyl has two rings which have three or more adjacent ring atoms in common and can be, e.g., a (4-13 membered) bridged bicyclic or (6-13 membered) bridged tricyclic such as adamantyl. The first ring attached to the parent molecular group is a monocyclic carbocyclyl and the second ring is also a monocyclic carbocyclyl.

A spiro bicyclic carbocyclyl has two rings which have only one ring atom in common and can be, e.g., a (6-13 membered) spiro bicyclic. The first ring attached to the parent molecular group is a monocyclic carbocyclyl and the second ring is also a monocyclic carbocyclyl.

Polycyclic carbocyclyls have more than two rings (e.g., three rings resulting in a tricyclic ring system) and adjacent rings have at least one ring atom in common. The first ring is a monocyclic carbocyclyl and the remainder of the ring structures are monocyclic carbocyclyls. Polycyclic ring systems include fused, bridged and spiro ring systems. A fused polycyclic ring system has at least two rings that have two adjacent ring atoms in common. A spiro polycyclic ring system has at least two rings that have only one ring atom in common. A bridged polycyclic ring system has at least two rings that have three or more adjacent ring atoms in common.

Suitable substituents for a “substituted carbocyclyls” include, but are not limited to halogen, —OH, —O—C₁-C₄ alkyl, C₁-C₄ alkyl, fluoro-substituted-C₁-C₄ alkyl, C₃-Cis carbocyclyl (e.g., cyclopropyl, cyclobutyl, cyclopentyl, cyclohexyl) phenyl, naphthalenyl, a (4-13 membered) heterocyclyl (e.g., pyrrolidine, piperidine, piperazine, tetrahydrofuran, tetrahydropyran or morpholine), or —N(R^{X})(R^{X′}), wherein R^{X} and R^{X′} are as described above.

“Cycloalkoxy” means a cycloalkyl radical attached through an oxygen linking atom. “(C₃-C₆)cycloalkoxy” includes cyclopropyloxy, cyclobutyloxy, cyclopentyloxy and cyclohexyloxy.

“Cycloalkene” means an aliphatic cyclic hydrocarbon ring having one or more double bonds in the ring.

“Cycloalkyne” means an aliphatic cyclic hydrocarbon ring having one or more triple bonds in the ring.

“Hetero” refers to the replacement of at least one carbon atom member in a ring system with at least one heteroatom selected from N, S, and O. “Hetero” also refers to the replacement of at least one carbon atom member in an acyclic system. When one heteroatom is S, it can be optionally mono- or di-oxygenated (i.e. —S(O)— or —S(O)₂—). A hetero ring system or a hetero acyclic system may have 1, 2, 3 or 4 carbon atom members replaced by a heteroatom.

“Heterocyclyl” means a cyclic 3-18 membered, for example 3-13-membered, 3-15, 5-18, 5-12, 3-12, 5-6 or 5-7-membered saturated or unsaturated aliphatic or aromatic ring system containing 1, 2, 3, 4 or 5 heteroatoms independently selected from N, O and S. When one heteroatom is S, it can be optionally mono- or di-oxygenated (i.e. —S(O)— or —S(O)₂—). The heterocyclyl can be monocyclic, fused bicyclic, bridged bicyclic, spiro bicyclic or polycyclic. Non-limiting examples include (4-7 membered) monocyclic, (6-13 membered) fused bicyclic, (6-13 membered) bridged bicyclic, or (6-13 membered) spiro bicyclic.

“Saturated heterocyclyl” means an aliphatic heterocyclyl group without any degree of unsaturation (i.e., no double bond or triple bond). It can be monocyclic, fused bicyclic, bridged bicyclic, spiro bicyclic or polycyclic.

Examples of monocyclic saturated heterocyclyls include, but are not limited to, azetidine, pyrrolidine, piperidine, piperazine, azepane, hexahydropyrimidine, tetrahydrofuran, tetrahydropyran, morpholine, thiomorpholine, thiomorpholine 1,1-dioxide, tetrahydro-2H-1,2-thiazine, tetrahydro-2H-1,2-thiazine 1,1-dioxide, isothiazolidine, isothiazolidine 1,1-dioxide.

One type of “heterocyclyl” is a “heteroaryl” or “heteroaromatic ring”, which refers to a 5-18 membered monovalent heteroaromatic monocyclic or bicylic ring radical. A heteroaryl contains 1, 2, 3 or 4 heteroatoms independently selected from N, O, and S.

A fused bicyclic heterocyclyl has two rings which have two adjacent ring atoms in common. The first ring is a monocyclic heterocyclyl and the second ring is a monocyclic carbocycle or a monocyclic heterocyclyl. For example, the second ring is a (C₃-C₆)cycloalkyl, such as cyclopropyl, cyclobutyl, cyclopentyl and cyclohexyl. Examples of fused bicyclic heterocyclyls include, but are not limited to, octahydrocyclopenta [c]pyrrolyl, indoline, isoindoline, 2,3-dihydro-1H-benzo[d]imidazole, 2,3-dihydrobenzo[d]oxazole, 2,3-dihydrobenzo[d]thiazole, octahydrobenzo[d]oxazole, octahydro-1H-benzo[d]imidazole, octahydrobenzo[d]thiazole, octahydrocyclopenta[c]pyrrole, 3-azabicyclo[3.1.0]hexane, and 3-azabicyclo[3.2.0]heptane.

A spiro bicyclic heterocyclyl has two rings which have only one ring atom in common. The first ring is a monocyclic heterocyclyl and the second ring is a monocyclic carbocycle or a monocyclic heterocyclyl. For example, the second ring is a (C₃-C₆)cycloalkyl. Examples of spiro bicyclic heterocyclyl includes, but are not limited to, azaspiro[4.4]nonane, 7-azaspiro[4.4]nonane, azasprio[4.5]decane, 8-azaspiro[4.5]decane, azaspiro[5.5]undecane, 3-azaspiro[5.5]undecane and 3,9-diazaspiro[5.5]undecane.

A bridged bicyclic heterocyclyl has two rings which have three or more adjacent ring atoms in common. The first ring is a monocyclic heterocyclyl and the other ring is a monocyclic carbocycle or a monocyclic heterocyclyl. Examples of bridged bicyclic heterocyclyls include, but are not limited to, azabicyclo[3.3.1]nonane, 3-azabicyclo[3.3.1]nonane, azabicyclo[3.2.1]octane, 3-azabicyclo[3.2.1]octane, 6-azabicyclo[3.2.1]octane and azabicyclo[2.2.2]octane, 2-azabicyclo[2.2.2]octane.

Polycyclic heterocyclyls have more than two rings, wherein the first ring can be a heterocyclyl (e.g., three rings resulting in a tricyclic ring system) and adjacent rings having at least one ring atom in common and are heterocyclyl or carbocyclyl. Polycyclic ring systems include fused, bridged and spiro ring systems. A fused polycyclic ring system has at least two rings that have two adjacent ring atoms in common. A spiro polycyclic ring system has at least two rings that have only one ring atom in common. A bridged polycyclic ring system has at least two rings that have three or more adjacent ring atoms in common. Examples of polycyclic heterocyclyls include

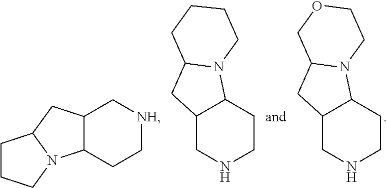

“Heteroaryl” or “heteroaromatic ring” means a 5-18 membered monovalent heteroaromatic monocyclic or bicylic ring radical. A heteroaryl contains 1, 2, 3 or 4 heteroatoms independently selected from N, O, and S. Heteroaryls include, but are not limited to furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, and thiazole. Bicyclic heteroaryl rings include, but are not limited to, bicyclo[4.4.0] and bicyclo[4.3.0] fused ring systems such as indolizine, indole, isoindole, indazole, benzimidazole, benzthiazole, purine, quinoline, isoquinoline, cinnoline, phthalazine, quinazoline, quinoxaline, 1,8-naphthyridine, and pteridine.

“Halogen” and “halo” are used interchangably herein and refer to fluorine, chlorine, bromine, or iodine.

“Haloalkyl” and “halocycloalkyl” include mono, poly, and perhaloalkyl groups where each halogen is independently selected from fluorine, chlorine, and bromine.

“Fluoro” means —F.

“Chloro” means —Cl.

As used herein, “fluoro-substituted-alkyl” or “fluoroalkyl” means an alkyl having a specified number of atoms and substituted with one or more —F groups. Examples of fluoro-substituted-alkyls include, but are not limited

to, —CF₃, —CH₂CF₃, —CH₂CF₂H, —CH₂CH₂F and —CH₂CH₂CF₃.

“Hydroxyalkyl,” as used herein, refers to an alkyl group substituted with one or more hydroxyls. Hydroxyalkyl includes mono, poly, and perhydroxyalkyl groups. Examples of hydroxyalkyls include —CH₂CH₂OH and —CH₂CH(OH)CH₂OH.

“Oxo” means substituted with ═O.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In the paragraphs below, where “Ph” is phenyl.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)_{0- 4}N(R^{◯}) C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C (O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C (O)SR^{◯}; —(CH₂)₀₋₄C(O)OSiR^{◯}₃; —(CH₂)₀₋₄OC(O)R^{◯}; —OC(O)(CH₂)₀₋₄SR—, —SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O) NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S (O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP (O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂S H, —(CH₂)₀₋₂NH₂, —(CH₂)₀₋₂NHR^{●}, —(CH₂)₀₋₂NR^{●}₂, —NO₂, —SiR^{●}3, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂N R^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Another embodiment of the present invention is a pharmaceutical composition comprising one or more pharmaceutically acceptable carrier and/or diluent and a compound disclosed herein, or a pharmaceutically acceptable salt thereof.

“Pharmaceutically acceptable carrier” and “pharmaceutically acceptable diluent” means non-therapeutic components that are of sufficient purity and quality for use in the formulation of a composition of the invention that, when appropriately administered to an animal or human, typically do not produce an adverse reaction, and that are used as a vehicle for a drug substance (i.e., a compound of the present invention).

Pharmaceutically acceptable salts of the compounds of the present invention are also included. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the compounds of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

The invention also includes various isomers and mixtures thereof. Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S, either a pure enantiomer or a mixture of both configurations is present.

“Racemate” or “racemic mixture” means a compound of equimolar quantities of two enantiomers, wherein such mixtures exhibit no optical activity; i.e., they do not rotate the plane of polarized light.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least about 60%, about 70%, about 80%, about 90%, about 99% or about 99.9% by weight pure relative to the other stereoisomers. When a single enantiomer is named or depicted by structure, the depicted or named enantiomer is at least about 60%, about 70%, about 80%, about 90%, about 99% or about 99.9% by weight optically pure. Percent optical purity by weight is the ratio of the weight of the enantiomer that is present divided by the combined weight of the enantiomer that is present and the weight of its optical isomer.

“Cis” means on the same side. “Trans” means on opposite sides. The designation “cis” is used when two substituents have an “up-up” or a “down-down” relationship. The designation “trans” is used when two substituents have an “up-down” or “down-up” relationship. Typically, two substituents that are “cis” to one another are arranged on the same side of a molecule. When the term “cis” is used with reference to a fused, saturated or partially saturated ring system, the term is intended to indicate that the two atoms attached to the common ring atoms are cis substituents. For example,

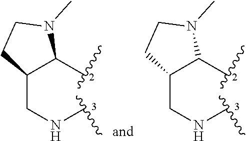

are cis diastereomers of a moiety having the following structural formula:

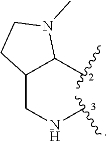 [SMILES: *NCC1CCN(C)C1*]

As used herein, the term “subject” means a mammal in need of treatment or prevention, e.g., a human, companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of the specified treatment.

As used herein, the term “treating” or ‘treatment” refers to obtaining desired pharmacological and/or physiological effect. The effect can include achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome.

As used herein, “preventing” or “prevention” refers to reducing the likelihood of the onset or development of disease, disorder or syndrome.

“Effective amount” means that amount of active compound agent that elicits the desired biological response in a subject. In one embodiment, the effective amount of a compound of the invention is from about 0.01 mg/kg/day to about 1000 mg/kg/day, from about 0.1 mg/kg/day to about 100 mg/kg/day, or from about 0.5 mg/kg/day to about 50 mg/kg/day.

As used herein the terms hematological malignancy and hematological cancer are used interchangeably and refer to cancers of the blood (leukemia) or cancers of the lymph system (lymphomas). Leukemias can include acute myeloid leukemia (AML), also known as acute myelogenous leukemia, acute myeloblastic leukemia, acute granulocytic leukemia or acute nonlymphocytic leukemia, acute lymphoblastic leukemia (ALL), chronic lymphocytic leukemia (CLL), chronic myelogenous leukemia (CML), chronic myelomonocytic leukemia (CMML), acute monocytic leukemia (AMoL). Lymphomas can include, Hodgkin's lymphoma, non-Hodgkin's lymphomas, multiple myeloma, myelodysplastic or myeloproliferative syndrome, mantle cell lymphoma, diffuse large B-cell lymphoma (DLBCL), Burkitt lymphoma/leukemia and B-cell lymphoma.

### Indications

Hematological malignancies are cancers that affect the blood and lymph system. Some types of hematologic malignancies include: Multiple myeloma, Hodgkin's lymphoma, Non-Hodgkin's lymphoma and Leukemia. The cancer may begin in blood-forming tissue (e.g., bone marrow), or in the cells of the immune system. For example, leukemia originates in blood-forming tissue. Leukemia is characterized by the uncontrolled growth of blood cells, usually white blood cells (leukocytes), in the bone marrow. White blood cells are a fundamental component of the body's immune response. The leukemia cells crowd out and replace normal blood and marrow cells.

There are four main types of leukemia: Acute myeloid leukemia (AML); Chronic myeloid leukemia (CML); Acute lymphocytic leukemia (ALL); and Chronic lymphocytic leukemia (CLL). The primary differences between the four main types of leukemia have to do with their rates of progression and where the cancer develops. Acute myeloid leukemia (AML), also known as acute myelogenous leukemia, acute myeloblastic leukemia, acute granulocytic leukemia or acute nonlymphocytic leukemia, is a fast-growing form of cancer of the blood and bone marrow. AML is the most common type of acute leukemia. It occurs when the bone marrow begins to make blasts, cells that have not yet completely matured. These blasts normally develop into white blood cells. However, in AML, these cells do not develop and are unable to ward off infections. In AML, the bone marrow may also make abnormal red blood cells and platelets. The number of these abnormal cells increases rapidly, and the abnormal (leukemia) cells begin to crowd out the normal white blood cells, red blood cells and platelets that the body needs.

In certain embodiments, provided herein is a method of treating a hematological cancer in a subject in need of treatment comprising administering to the subject in need of treatment an effective among of any of the compounds disclosed herein, including a compound of Formula (I), Formula (I′), Formula (II), Formula (II′), Formula (III), Formula (III′), Formula (IV), Formula (IV′), Formula (V), Formula (V′), Formula (VI), Formula (VI′), Formula (VII) or Formula (VII′), or a pharmaceutically acceptable salt thereof or a pharmaceutically acceptable composition thereof. In further embodiments, provided herein is a method of treating a hematological cancer in a subject in need of treatment comprising administering to the subject in need of treatment an effective among of any of the compounds disclosed herein, including a compound of Formula (X), (X-1), (XI), (XII), (XX), (XXI), (XXII), (XXIII), (XXIV), (XXV), (XIII), (XIV), or (XV).

In one aspect, the hematological cancer is selected from Acute Myeloid Leukemia, Multiple myeloma, Hodgkin lymphoma, Non-Hodgkin lymphoma and Leukemia

In particular embodiments, provided herein is a method of treating a leukemia in a subject in need of treatment comprising administering to the subject in need of treatment an effective among of any of the compounds disclosed herein, including a compound of Formula (I), Formula (I′), Formula (II), Formula (II′), Formula (III), Formula (III′), Formula (IV), Formula (IV′), Formula (V), Formula (V′), Formula (VI), Formula (VI′), Formula (VII) or Formula (VII′), or a pharmaceutically acceptable salt thereof or a pharmaceutically acceptable composition thereof. In further embodiments, provided herein is a method of treating a leukemia in a subject in need of treatment comprising administering to the subject in need of treatment an effective among of any of the compounds disclosed herein, including a compound of Formula (X), (X-1), (XI), (XII), (XX), (XXI), (XXII), (XXIII), (XXIV), (XXV), (XIII), (XIV), or (XV).

In some embodiments, provided herein is a method of treating acute myeloid leukemia in a subject in need of treatment comprising administering to the subject an effective amount of any of the compounds disclosed herein, including a compound of Formula (I), Formula (I′), Formula (II), Formula (II′), Formula (III), Formula (III′), Formula (IV), Formula (IV′), Formula (V), Formula (V′), Formula (VI), Formula (VI′), Formula (VII) or Formula (VII′), or a pharmaceutically acceptable salt thereof or a pharmaceutically acceptable composition thereof. In some embodiments, provided herein is a method of treating acute myeloid leukemia in a subject in need of treatment comprising administering to the subject an effective amount of any of the compounds disclosed herein, including a compound of Formula (X), (X-1), (XI), (XII), (XX), (XXI), (XXII), (XXIII), (XXIV), (XXV), (XIII), (XIV), or (XV).

In certain embodiments, provided herein is a method of treating acute myeloid leukemia comprising administering to a subject an effective amount of a compound of Formula (I), Formula (I′), or a pharmaceutically acceptable salt thereof or a pharmaceutically acceptable composition thereof. In one aspect of this embodiment, the compound is selected from Compounds 3, 3a, 3b, 4, 4a, 4b and 5 as defined herein or a pharmaceutically acceptable salt thereof. In a specific aspect, the compound is Compound 3a.

In certain embodiments, provided herein is a method of treating acute myeloid leukemia in a subject in need of treatment comprising administering to the subject an effective amount of a compound of Formula (II), Formula (II′), or a pharmaceutically acceptable salt thereof or a pharmaceutically acceptable composition thereof.

In certain embodiments, provided herein is a method of treating acute myeloid leukemia in a subject in need of treatment comprising administering to the subject an effective amount of a compound of Formula (III), Formula (III′) or a pharmaceutically acceptable salt thereof or a pharmaceutically acceptable composition thereof. In one aspect of this embodiment, the compound is selected from Compounds 1 and 2 as described herein or a pharmaceutically acceptable salt thereof.

In certain embodiments, provided herein is a method of treating acute myeloid leukemia in a subject in need of treatment comprising administering to the subject an effective amount of a compound of Formula (IV), Formula (IV′) or a pharmaceutically acceptable salt thereof or a pharmaceutically acceptable composition thereof.

In other embodiments, provided herein is a method of treating acute myeloid leukemia in a subject in need thereof comprising administering to the subject an effective amount of a compound of Formula (V), Formula (V′) or a pharmaceutically acceptable salt thereof or a pharmaceutically acceptable composition thereof.

In certain embodiments, provided herein is a method of treating acute myeloid leukemia in a subject in need of treatment comprising administering to the subject an effective amount of a compound of Formula (VI), Formula (VI′) or a pharmaceutically acceptable salt thereof or a pharmaceutically acceptable composition thereof.

In certain embodiments, provided herein is a method of treating acute myeloid leukemia in a subject in need of treatment comprising administering to the subject an effective amount of a compound of Formula (VII), Formula (VII′) or a pharmaceutically acceptable salt thereof or a pharmaceutically acceptable composition thereof.

In some embodiments, the compound of Formula (I) is a compound selected from formulae (Ia), (Ia′), (Ib), (Ib′), (Ic), (Ic′), (Ic-1), (Ic′-1), (Id), (Id′), (Ie) and (Ie′). In some embodiments, the compound of Formula (II) is a compound selected from formulae (IIa), (IIa′), (IIa-1), (IIa′-1), (IIb), (IIb′), (IIb-1), (IIb′-1), (IIb-2), (IIb′-2), (IIc), (IIc′), (IIc-1), (IIc′-1), (IId) and (IId′). In some embodiments, the compound is selected from Formula (III), Formula (III′), Formula (IV), Formula (IV′), Formula (V), Formula (V′), Formula (VI), Formula (VI′), Formula (VII) and Formula (VII′).

In some embodiments, the methods described herein comprise administering to a subject in need of treatment an effective amount of a compound selected from Compound 1, Compound 2, Compound 3, Compound 3a, Compound 3b, Compound 4, Compound 4a, Compound 4b and Compound 5.

In certain embodiments, the compound is Compound 1. In certain embodiments, the compound is Compound 2. In certain embodiments, the compound is Compound 3a. In certain embodiments, the compound is Compound 4a. In certain embodiments, the compound is Compound 5.

In other embodiments, provided herein is the use of an effective amount of a compound of Formula (I), Formula (I′), Formula (II), Formula (II′), Formula (III), Formula (III′), Formula (IV), Formula (IV′), Formula (V), Formula (V′), Formula (VI), Formula (VI′), Formula (VII) or Formula (VII′), or a pharmaceutically acceptable salt thereof or a pharmaceutically acceptable composition thereof, in the manufacture of a medicament for the treatment of a hematological cancer. In one aspect, the hematological cancer is Multiple myeloma, Hodgkin lymphoma, Non-Hodgkin lymphoma and Leukemia. In a particular aspect the hematological cancer is leukemia. In a more particular aspect, the leukemia is acute myeloid leukemia. All compound and Formula embodiments described above are contemplated for these uses.

In other embodiments, provided herein is the use of an effective amount of a compound of Formula (I), Formula (I′), Formula (II), Formula (II′), Formula (III), Formula (III′), Formula (IV), Formula (IV′), Formula (V), Formula (V′), Formula (VI), Formula (VI′), Formula (VII), Formula (VII′), Formula (X), Formula (X-1), Formula (XI), Formula (XII), Formula (XX), Formula (XXI), Formula (XXII), Formula (XXIII), Formula (XXIV), Formula (XXV), Formula (XIII), Formula (XIV), or Formula (XV).

or a pharmaceutically acceptable salt thereof or a pharmaceutically acceptable composition thereof, for the treatment of a hematological cancer. In one aspect, the hematological cancer is Multiple myeloma, Hodgkin lymphoma, Non-Hodgkin lymphoma and Leukemia. In a particular aspect the hematological cancer is leukemia. In a more particular aspect, the leukemia is acute myeloid leukemia.

All compound and Formulas described above are contemplated for these uses.

### Bacterial Infections

Compounds of the invention, in particular, a compound represented by any one of structural formulas XV or XIV or a compound of Formulas XIII or XII, can be used to prevent or treat important mammalian and veterinary diseases such as diarrhea, urinary tract infections, infections of skin and skin structure including wounds, cellulitis, and abscesses, ear, nose and throat infections, mastitis and the like. In addition, methods for treating neoplasms using tetracycline compounds of the invention are also included (van der Bozert et al., Cancer Res., 48: 6686-6690 (1988)).

Infections that can be treated using compounds of the invention or a pharmaceutically acceptable salt thereof include, but are not limited to, skin infections, GI infections, urinary tract infections, genito-urinary infections, respiratory tract infections, sinuses infections, middle ear infections, systemic infections, intra-abdominal infections, pyelonephritis, pneumonia, bacterial vaginosis, streptococcal sore throat, chronic bacterial prostatitis, gynecological and pelvic infections, sexually transmitted bacterial diseases, ocular and otic infections, cholera, influenza, bronchitis, acne, psoriasis, rosacea, impetigo, malaria, sexually transmitted disease including syphilis and gonorrhea, Legionnaires' disease, Lyme disease, Rocky Mountain spotted fever, Q fever, typhus, bubonic plague, gas gangrene, hospital acquired infections, leptospirosis, whooping cough, anthrax and infections caused by the agents responsible for lymphogranuloma venereum, inclusion conjunctivitis, or psittacosis. Infections can be bacterial, fungal, parasitic and viral infections (including those which are resistant to other tetracycline compounds).

In one embodiment, the infection is a respiratory infection. In a particular aspect, the respiratory infection is Community-Acquired Bacterial Pneumonia (CABP). In a more particular embodiment, the respiratory infection, for example, CABP is caused by a bacterium selected from S. aureus, S. pneumoniae, S. pyogenes, H. influenza, M catarrhalis and Legionella pneumophila.

In another embodiment, the infection is a skin infection. In a particular aspect the skin infection is an acute bacterial skin and skin structure infection (ABSSSI). In a more particular embodiment, the skin infection, for example ABSSSI is caused by a bacterium selected from S. aureus, CoNS, S. pyogenes, S. agalactiae, E. faecalis and E. faecium.

In one embodiment, the infection can be caused by a bacterium (e.g. an anaerobic or aerobic bacterium).

In another embodiment, the infection is caused by a Gram-positive bacterium. In a specific aspect of this embodiment, the infection is caused by a Gram-positive bacterium selected from class Bacilli, including, but not limited to, Staphylococcus spp., Streptococcus spp., Enterococcus spp., Bacillus spp., Listeria spp.; phylum Actinobacteria, including, but not limited to, Propionibacterium spp., Corynebacterium spp., Nocardia spp., Actinobacteria spp., and class Clostridia, including, but not limited to, Clostridium spp.

In another embodiment, the infection is caused by a Gram-positive bacterium selected from S. aureus, CoNS, S. pneumoniae, S. pyogenes, S. agalactiae, E. faecalis and E. faecium.

In another embodiment, the infection is caused by a Gram-negative bacterium. In one aspect of this embodiment, the infection is caused by a phylum Proteobacteria (e.g., Betaproteobacteria and Gammaproteobacteria), including Escherichia coli, Salmonella, Shigella, other Enterobacteriaceae, Pseudomonas, Moraxella, Helicobacter, Stenotrophomonas, Bdellovibrio, acetic acid bacteria, Legionella or alpha-proteobacteria such as Wolbachia. In another aspect, the infection is caused by a Gram-negative bacterium selected from cyanobacteria, spirochaetes, green sulfur or green non-sulfur bacteria. In a specific aspect of this embodiment, the infection is caused by a Gram-negative bacteria selected from Enterobactericeae (e.g., E. coli, Klebsiella pneumoniae including those containing extended-spectrum β-lactamases and/or carbapenemases), Bacteroidetes (e.g., Bacteroides fragilis)., Vibrionaceae (Vibrio cholerae), Pasteurellaceae (e.g., Haemophilus influenzae), Pseudomonadaceae (e.g., Pseudomonas aeruginosa), Neisseriaceae (e.g. Neisseria meningitidis), Rickettsiae, Moraxellaceae (e.g., Moraxella catarrhalis), any species of Proteeae, Acinetobacter spp., Helicobacter spp., and Campylobacter spp. In a particular embodiment, the infection is caused by Gram-negative bacterium selected from the group consisting of Enterobactericeae (e.g., E. coli, Klebsiella pneumoniae), Pseudomonas, and Acinetobacter spp. In another embodiment, the infection is caused by an organism selected from the group consisting of K. pneumoniae, Salmonella, E. hirae, A. baumanii, M catarrhalis, H. influenzae, P. aeruginosa, E. faecium, E. coli, S. aureus, and E. faecalis.

In another embodiment, the infection is cause by a gram negative bacterium selected from H. influenza, M catarrhalis and Legionella pneumophila.

In one embodiment, the infection is caused by an organism that grows intracellularly as part of its infection process.

In another embodiment, the infection is caused by an organism selected from the group consisting of order Rickettsiales; phylum Chlamydiae; order Chlamydiales; Legionella spp.; class Mollicutes, including, but not limited to, Mycoplasma spp. (e.g. Mycoplasma pneumoniae); Mycobacterium spp. (e.g. Mycobacterium tuberculosis); and phylum Spriochaetales (e.g. Borrelia spp. and Treponema spp.).

In another embodiment, the infection is caused by a Category A Biodefense organism as described at http://www.bt.cdc.gov/agent/agentlist-category.asp, the entire teachings of which are incorporated herein by reference. Examples of Category A organisms include, but are not limited to, Bacillus anthracis (anthrax), Yersinia pestis (plague), Clostridium botulinum (botulism) or Francisella tularensis (tularemia). In another embodiment the infection is a Bacillus anthracis infection. “Bacillus anthracis infection” includes any state, diseases, or disorders caused or which result from exposure or alleged exposure to Bacillus anthracis or another member of the Bacillus cereus group of bacteria.

Additional infections that can be treated using compounds of the invention or a pharmaceutically acceptable salt thereof include, but are not limited to, anthrax, botulism, bubonic plague, and tularemia.

In another embodiment, the infection is caused by a Category B Biodefense organism as described at http://www.bt.cdc.gov/agent/agentlist-category.asp, the entire teachings of which are incorporated herein by reference. Examples of Category B organisms include, but are not limited to, Brucella spp, Clostridium perfringens, Salmonella spp., Escherichia coli O157:H7, Shigella spp., Burkholderia mallei, Burkholderia pseudomallei, Chlamydia psittaci, Coxiella burnetii, Staphylococcal enterotoxin B, Rickettsia prowazekii, Vibrio cholerae, and Cryptosporidium parvum.

Additional infections that can be treated using compounds of the invention or a pharmaceutically acceptable salt thereof include, but are not limited to, Brucellosis, Clostridium perfringens, food-borne illnesses, Glanders, Melioidosis, Psittacosis, Q fever, and water-borne illnesses.

In yet another embodiment, the infection can be caused by one or more than one organism described above. Examples of such infections include, but are not limited to, intra-abdominal infections (often a mixture of a gram-negative species like E. coli and an anaerobe like B. fragilis), diabetic foot (various combinations of Streptococcus, Serratia, Staphylococcus and Enterococcus spp., anaerobes (S. E. Dowd, et al., PloS one 2008; 3:e3326, the entire teachings of which are incorporated herein by reference) and respiratory disease (especially in patients that have chronic infections like cystic fibrosis—e.g., S. aureus plus P. aeruginosa or H. influenzae, atypical pathogens), wounds and abscesses (various gram-negative and gram-positive bacteria, notably MSSA/MRSA, coagulase-negative staphylococci, enterococci, Acinetobacter, P. aeruginosa, E. coli, B. fragilis), and bloodstream infections (13% were polymicrobial (H. Wisplinghoff, et al., Clin. Infect. Dis. 2004; 39:311-317, the entire teachings of which are incorporated herein by reference)).

In one embodiment, the infection is caused by an organism resistant to one or more antibiotics.

In another embodiment, the infection is caused by an organism resistant to tetracycline or any member of first and second generation of tetracycline antibiotics (e.g., doxycycline or minocycline).

In another embodiment, the infection is caused by an organism resistant to methicillin.

In another embodiment, the infection is caused by an organism resistant to vancomycin.

In another embodiment, the infection is caused by an organism resistant to a quinolone or fluoroquinolone.

In another embodiment, the infection is caused by an organism resistant to tigecycline or any other tetracycline derivative. In a particular embodiment, the infection is caused by an organism resistant to tigecycline.

In another embodiment, the infection is caused by an organism resistant to a β-lactam or cephalosporin antibiotic or an organism resistant to penems or carbapenems.

In another embodiment, the infection is caused by an organism resistant to an antimicrobial peptide or a biosimilar therapeutic treatment. Antimicrobial peptides (also called host defense peptides) are an evolutionarily conserved component of the innate immune response and are found among all classes of life. In this case, antimicrobial peptide refers to any naturally occurring molecule or any semi/synthetic molecule that are analogs of anionic peptides, linear cationic α-helical peptides, cationic peptides enriched for specific amino acids (i.e, rich in proline, arginine, phenylalanine, glycine, tryptophan), and anionic and cationic peptides that contain cystein and form disulfide bonds.

In another embodiment, the infection is caused by an organism resistant to macrolides, lincosamides, streptogramin antibiotics, oxazolidinones, and pleuromutilins.

In another embodiment, the infection is caused by an organism resistant to PTK0796 (7-dimethylamino, 9-(2,2-dimethyl-propyl)-aminomethylcycline).

In another embodiment, the infection is caused by a multidrug-resistant pathogen (having intermediate or full resistance to any two or more antibiotics).

### Cancer Combination Therapies

In some embodiments, a compound described herein is administered together with an additional cancer treatment. Exemplary cancer treatments include, for example, chemotherapy, targeted therapies such as antibody therapies, kinase inhibitors, immunotherapy, and hormonal therapy, and anti-angiogenic therapies. Examples of each of these treatments are provided below.

As used herein, the term “combination,” “combined,” and related terms refer to the simultaneous or sequential administration of therapeutic agents in accordance with this invention. For example, a compound of the present invention can be administered with another therapeutic agent simultaneously or sequentially in separate unit dosage forms or together in a single unit dosage form. Accordingly, the present invention provides a single unit dosage form comprising a compound of the invention, an additional therapeutic agent, and a pharmaceutically acceptable carrier, adjuvant, or vehicle.

The amount of both a compound of the invention and additional therapeutic agent (in those compositions which comprise an additional therapeutic agent as described above) that can be combined with the carrier materials to produce a single dosage form will vary depending upon the host treated and the particular mode of administration. For example, compositions of this invention should be formulated so that a dosage of between 0.01-100 mg/kg body weight/day of a compound of the invention can be administered.

### Chemotherapy

In some embodiments, a compound described herein is administered with a chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, topoisomerase inhibitors and others). Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, Bendamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time. Often, two or more chemotherapy agents are used as combination chemotherapy. In some embodiments, the chemotherapy agents (including combination chemotherapy) can be used in combination with a compound described herein.

In a specific embodiment, the two additional therapeutic agents used in combination with the compounds of the invention and include, cytarabine (ara-C) and an anthracycline drug such as daunorubicin (daunomycin) or idarubicin. In certain instances, a third additional agent, cladribine, is used.

### Targeted Therapy

Targeted therapy constitutes the use of agents specific for the deregulated proteins of cancer cells. Small molecule targeted therapy drugs are generally inhibitors of enzymatic domains on mutated, overexpressed, or otherwise critical proteins within a cancer cell. Prominent examples are the tyrosine kinase inhibitors such as axitinib, bosutinib, cediranib, desatinib, erolotinib, imatinib, gefitinib, lapatinib, lestaurtinib, nilotinib, semaxanib, sorafenib, sunitinib, and vandetanib, and also cyclin-dependent kinase inhibitors such as alvocidib and seliciclib. Monoclonal antibody therapy is another strategy in which the therapeutic agent is an antibody which specifically binds to a protein on the surface of the cancer cells. Examples include the anti-HER2/neu antibody trastuzumab (Herceptin®) typically used in breast cancer, and the anti-CD20 antibody rituximab and tositumomab typically used in a variety of B-cell malignancies. Other exemplary antibodies include cetuximab, panitumumab, trastuzumab, alemtuzumab, bevacizumab, edrecolomab, and gemtuzumab. Exemplary fusion proteins include aflibercept and denileukin diftitox. In some embodiments, targeted therapy can be used in combination with a compound described herein, e.g., Gleevec (Vignari and Wang 2001).

Targeted therapy can also involve small peptides as “homing devices” which can bind to cell surface receptors or affected extracellular matrix surrounding a tumor. Radionuclides which are attached to these peptides (e.g., RGDs) eventually kill the cancer cell if the nuclide decays in the vicinity of the cell. An example of such therapy includes BEXXAR®.

### Pharmaceutical Formulations

The compositions of the invention include ocular, oral, nasal, transdermal, topical with or without occlusion, intravenous (both bolus and infusion), inhalable, and injection (intraperitoneally, subcutaneously, intramuscularly, intratumorally, or parenterally) formulations. The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains an effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., about 500 to about 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles or nanoemulsions (e.g., in the range of about 1 to about 500 nm in diameter, preferably about 50 to about 200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound disclosed herein may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from about 0.005 to about 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

In another embodiment, the compounds of this invention may be administered directly to the lungs by inhalation.

Compounds of the invention may also be administered topically or enhanced by using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

While this invention has been particularly shown and described with references to example embodiments thereof, it will be understood by those skilled in the art that various changes in form and details may be made therein without departing from the scope of the invention encompassed by the appended claims.

### EXEMPLIFICATION

Additional methods of synthesizing the compounds described herein and their synthetic precursors are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

### Example 1: Synthesis of Compounds 1 through 5

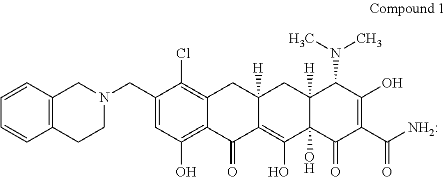

Compound 1 was prepared according to the synthesis described in WO2010/129057 at pp. 69-70 (S15-13-190), incorporated herein by reference in its entirety.

¹H NMR (400 MHz, CD₃OD) δ 7.34-7.24 (comp, 4H), 7.21-7.17 (m, 1H), 4.69 (s, 2 H), 4.54 (s, 2H), 4.11 (s, 1H), 3.90-3.53 (m, 2H), 3.47-3.39 (m, 2H), 3.04 (s, 3H), 2.96 (s, 3H), 3.28-2.94 (comp, 3H), 2.50-2.40 (m, 1H), 2.29-2.22 (m, 1H), 1.72-1.61 (m, 1H); MS (ESI) m/z 594.15 (M+H).

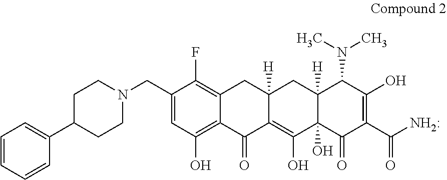

Compound 2 was prepared according to the synthesis described in WO2010/129057 at pp. 248-249 (S1-14-60).

¹H NMR (400 MHz, CD₃OD) δ 7.24-7.11 (m, 5H), 7.07 (d, J=4.8 Hz, 1H), 4.35 (s, 2H), 4.04 (s, 1H), 3.60-3.57 (m, 3H), 3.16-2.80 (m, 11H), 2.31-2.17 (m, 2H), 2.06-1.96 (s, 4H), 1.63-1.52 (m, 1H); MS (ESI) m/z 606.2 (M+H).

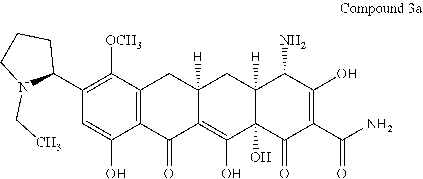 [SMILES: CCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

Compound 3a was prepared according to the synthesis described in WO2014/036502 at p. 142 (S10-4-1), incorporated herein by reference in its entirety.

¹H NMR (400 MHz, CD₃OD, hydrochloride salt) δ 7.09 (s, 1H), 3.90 (s, 1H), 3.86-3.80 (m, 1H), 3.68 (s, 3H), 3.37-3.30 (m, 1H), 3.28-3.07 (m, 3H), 3.00-2.91 (m, 1H), 2.67-2.54 (m, 2H), 2.41 (t, J=14.2 Hz, 1H), 2.34-2.21 (m, 5H), 1.66-1.57 (m, 1H), 1.25 (t, J=7.3 Hz, 3H); MS (ESI) m/z 514.28 (M+H).

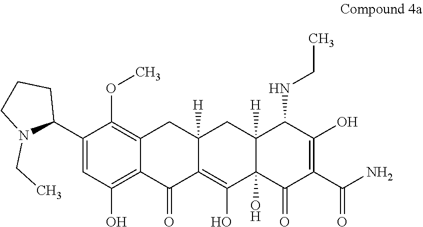 [SMILES: CCN[C@@H]1C(O)=C(C(N)=O)C(=O)[C@@]2(O)C(O)=C3C(=O)c4c(O)cc([C@@H]5CCCN5CC)c(OC)c4C[C@H]3C[C@@H]12]

Compound 4a was prepared according to the synthesis described in WO2014/036502, at pp 142-143 (S10-4-2).

(single diastereomer): ¹H NMR (400 MHz, CD₃OD, hydrochloride salt) δ 7.10 (s, 1H), 3.88 (s, 1H), 3.85-3.80 (m, 1H), 3.68 (s, 3H), 3.46-3.31 (m, 3H), 3.27-3.07 (m, 3H), 3.01-2.92 (m, 1H), 2.86-2.83 (m, 1H), 2.62-2.55 (m, 1H), 2.39 (t, J=14.2 Hz, 1H), 2.34-2.22 (m, 5H), 1.64-1.55 (m, 1H), 1.36 (t, J=7.3 Hz, 3H), 1.25 (t, J=7.3 Hz, 3H); MS (ESI) m/z 542.35 (M+H).

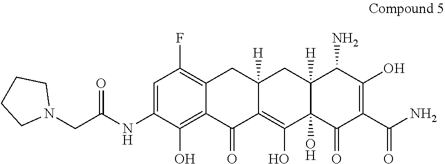 [SMILES: NC(=O)C1=C(O)[C@@H](N)[C@@H]2C[C@@H]3Cc4c(F)cc(NC(=O)CN5CCCC5)c(O)c4C(=O)C3=C(O)[C@]2(O)C1=O]

Compound 5 was prepared according to the synthesis described in WO2014/036502 at p. 140 (S9-5-4).

¹H NMR (400 MHz, CD₃OD, hydrochloride salt) δ 8.22 (d, J=11.0 Hz, 1H), 4.33 (s, 2H), 3.89 (s, 1H), 3.82-3.76 (m, 2H), 3.23-3.12 (m, 3H), 3.02-2.94 (m, 1H), 2.67-2.64 (m, 1H), 2.32-2.14 (m, 4H), 2.12-2.02 (m, 2H), 1.63-1.54 (m, 1H); MS (ESI) m/z 531.31 (M+H).

Compounds 1, 2, 3a, 4a and 5 are also referred to herein as Compounds K11, K31, K4, K5 and K43.

### Example 2: Anti-Cancer Activity of Compound 1-5

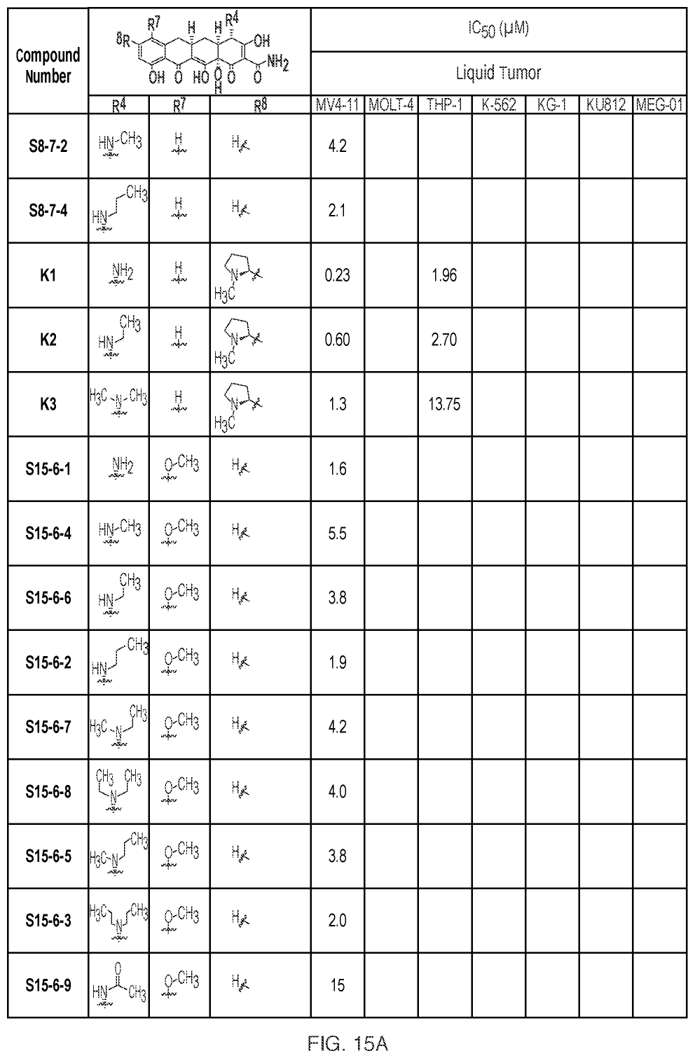

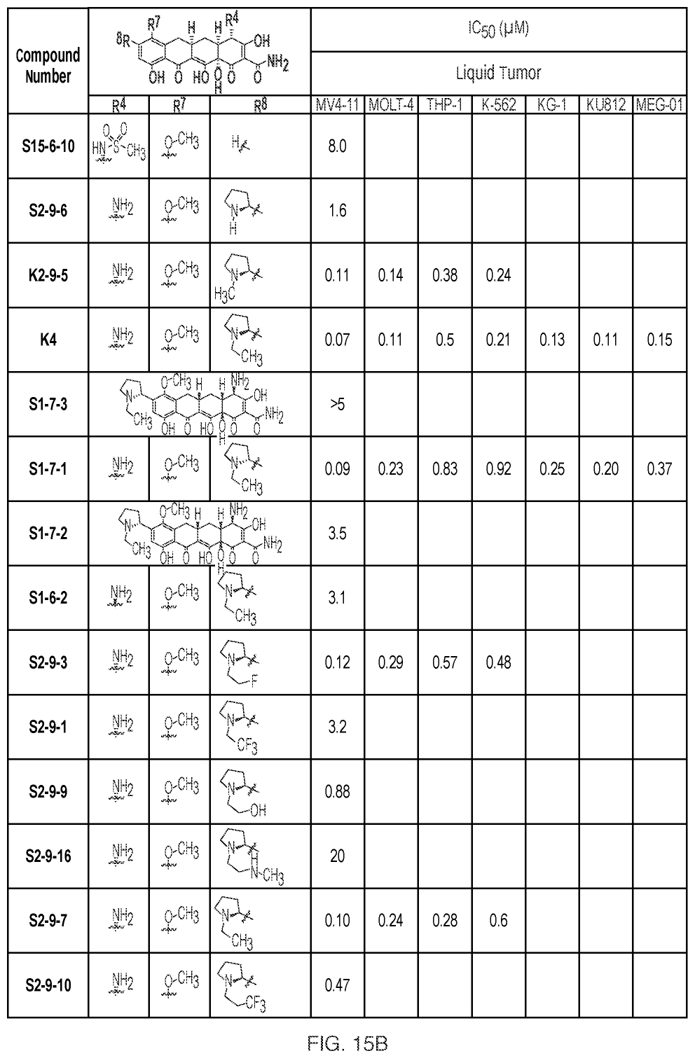

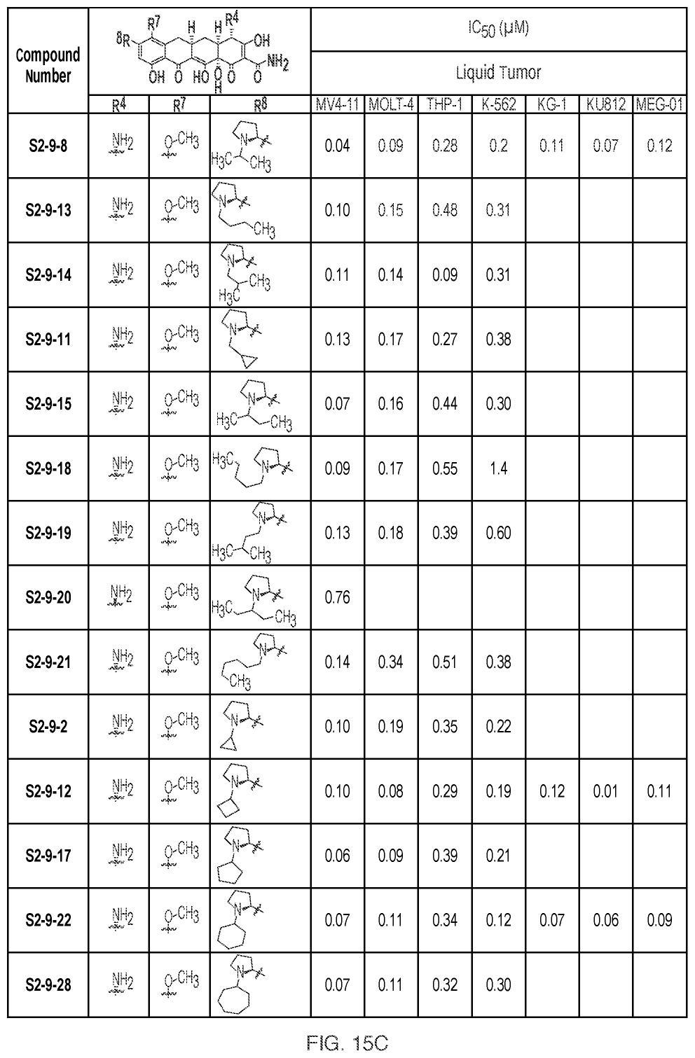

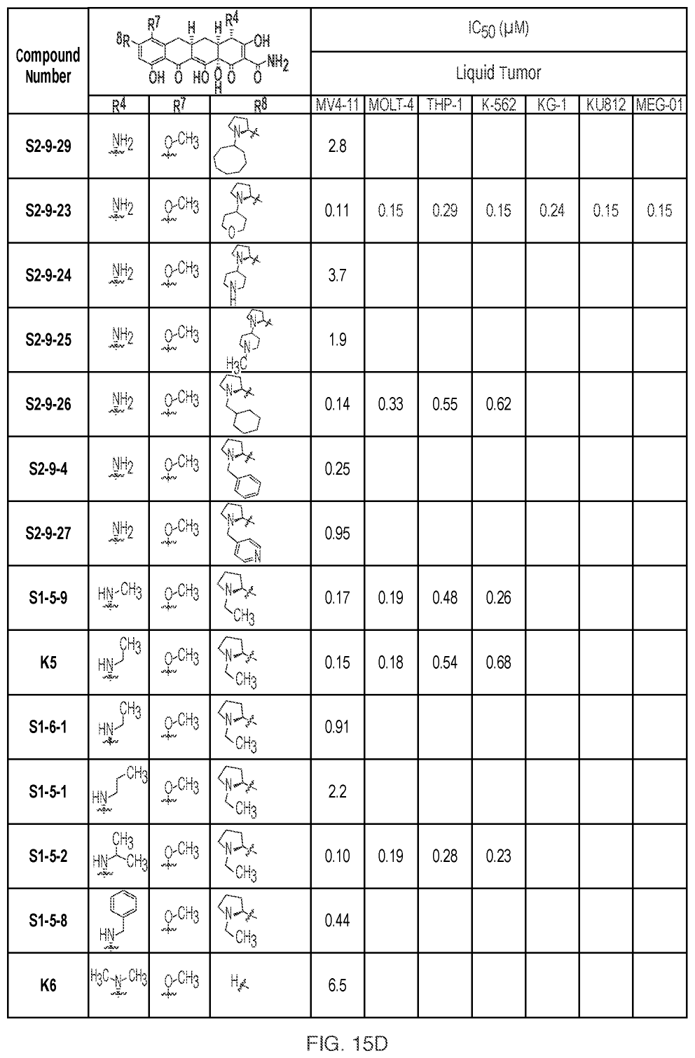

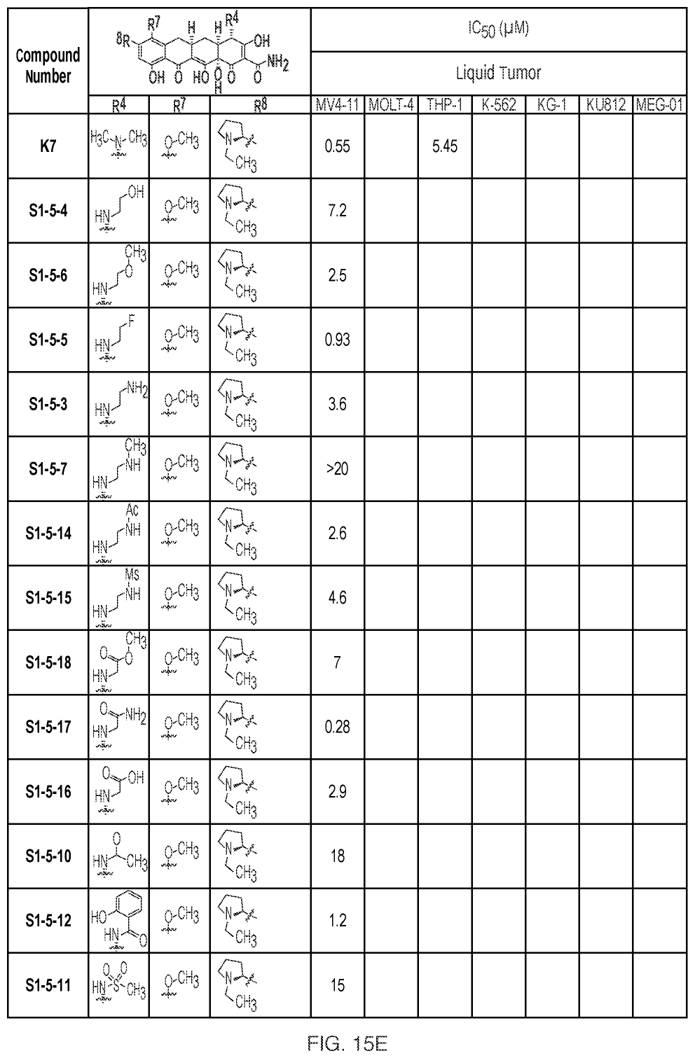

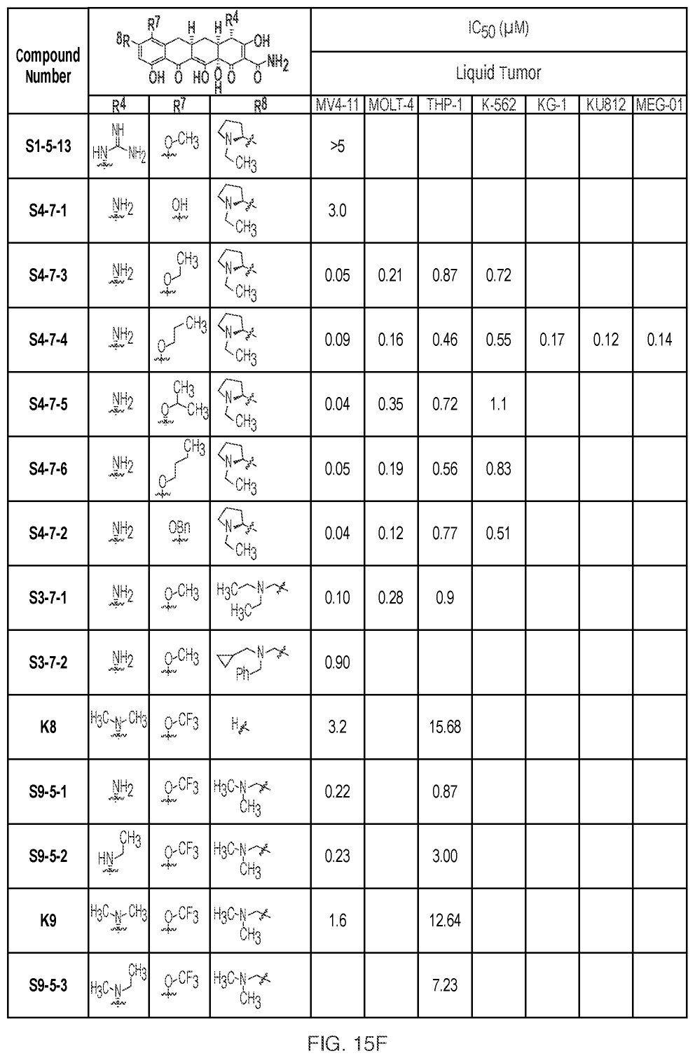

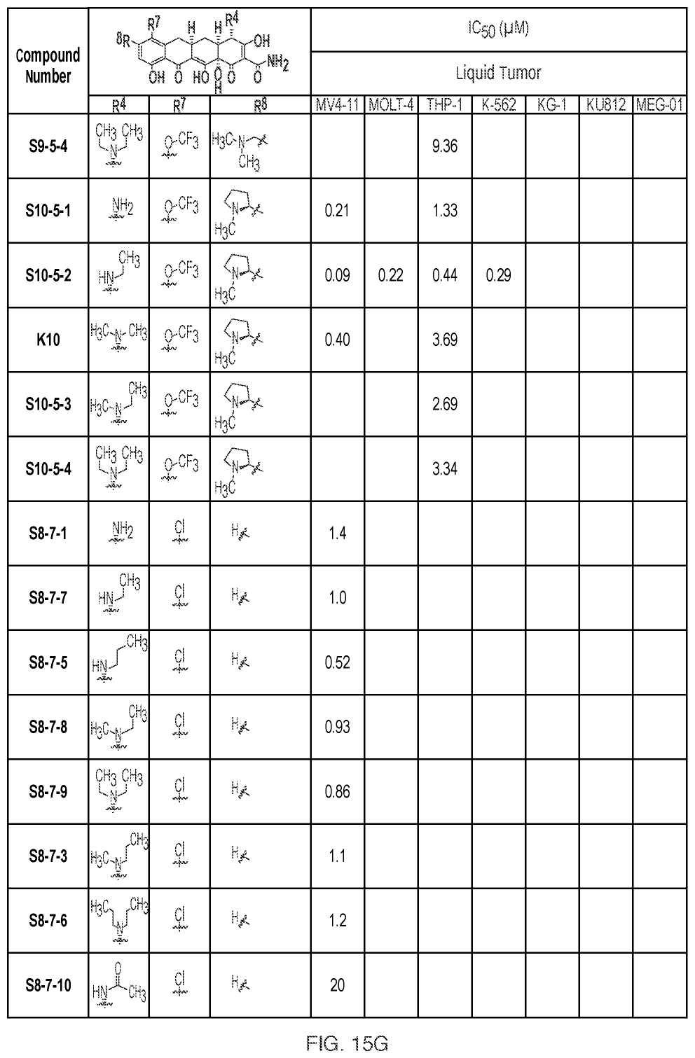

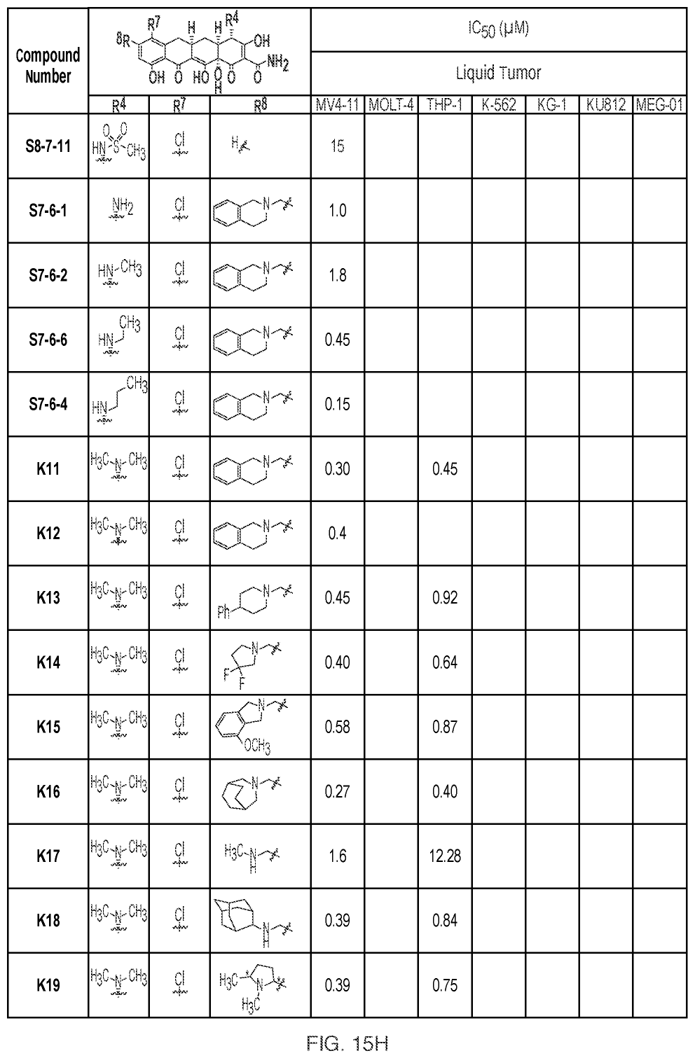

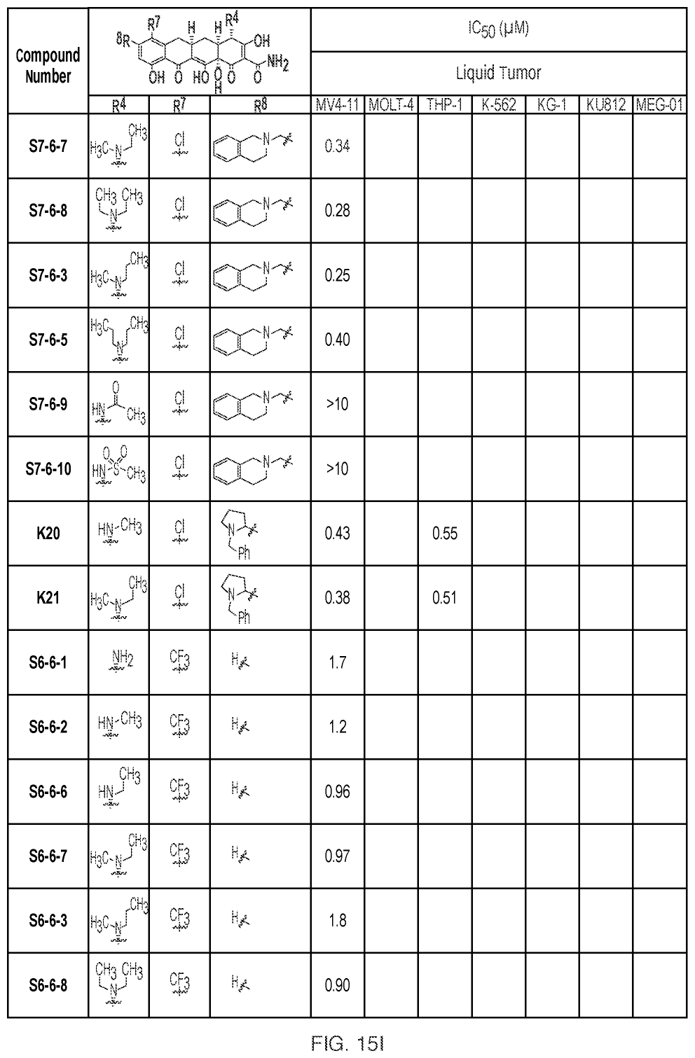

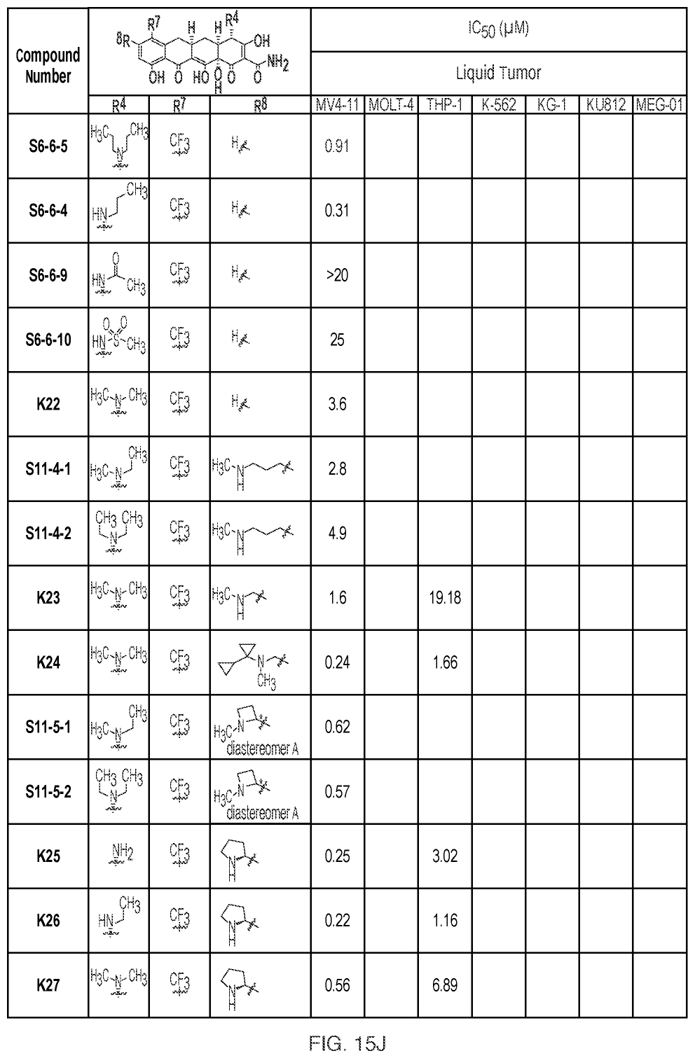

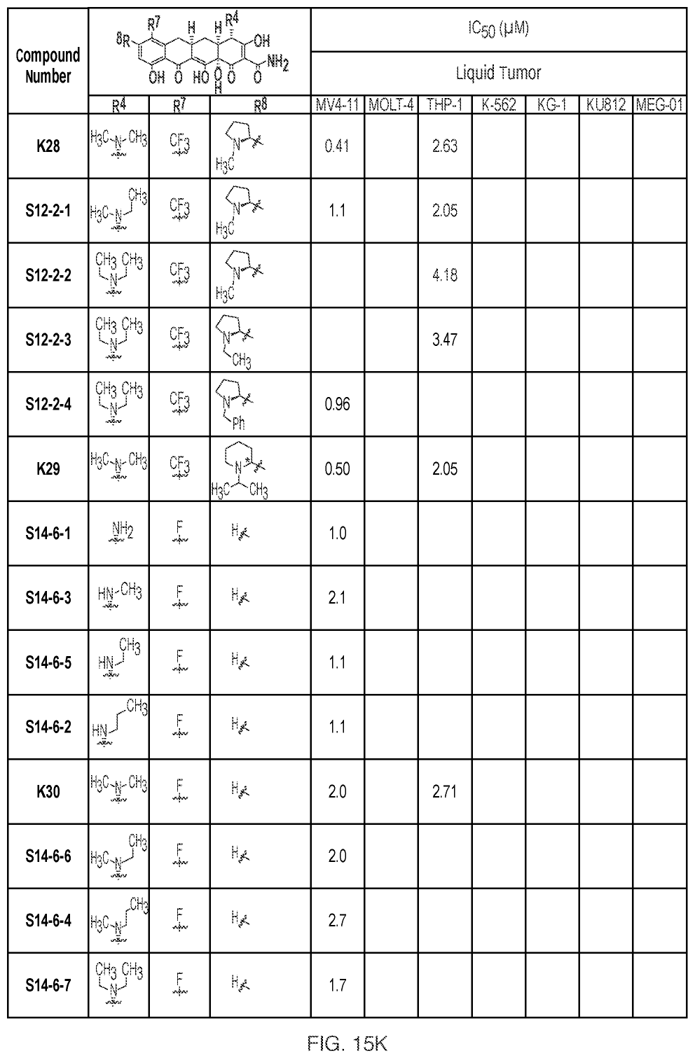

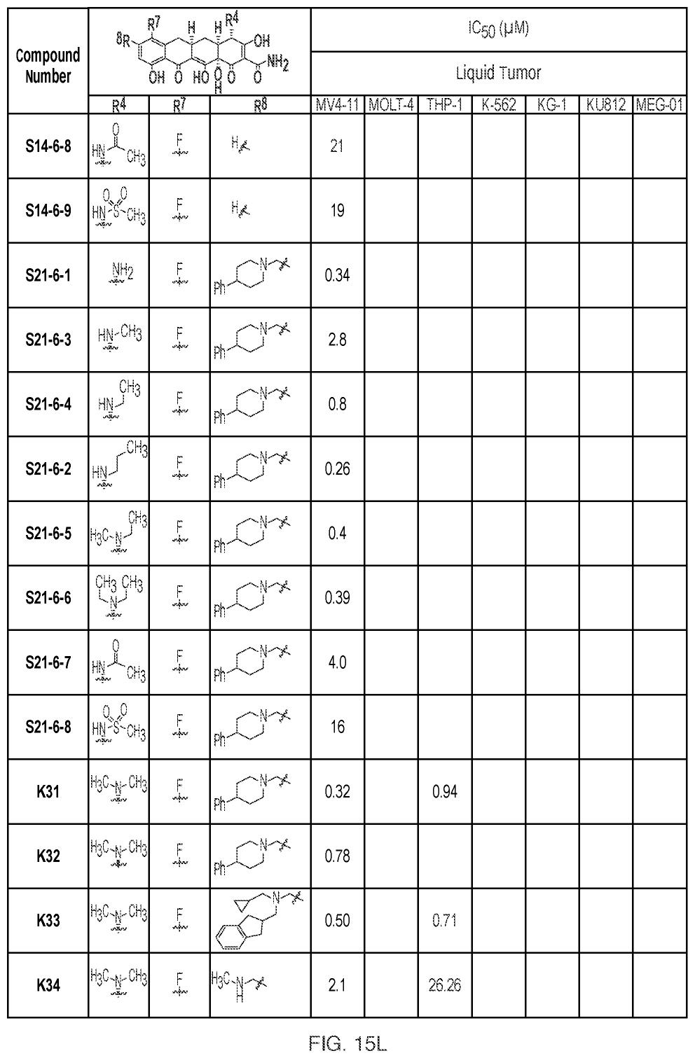

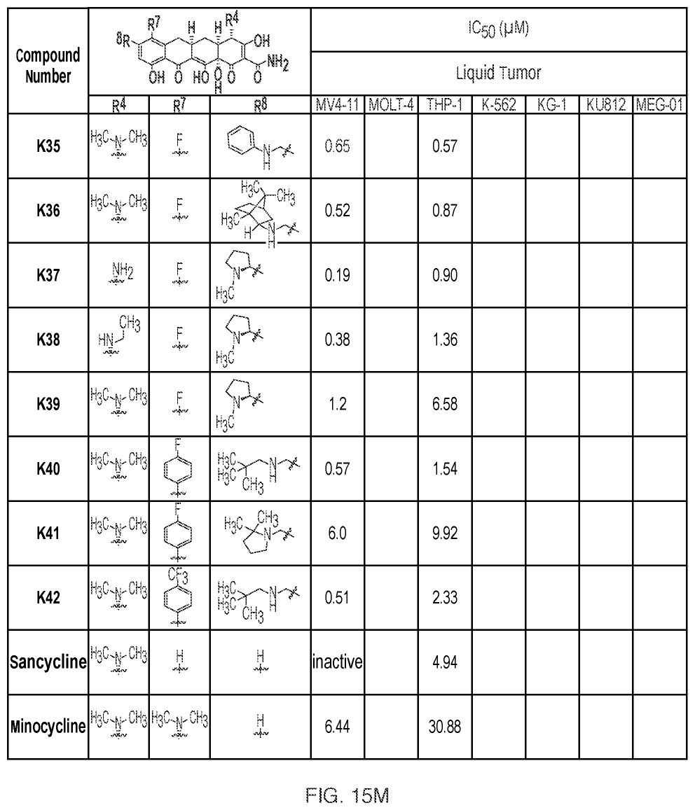

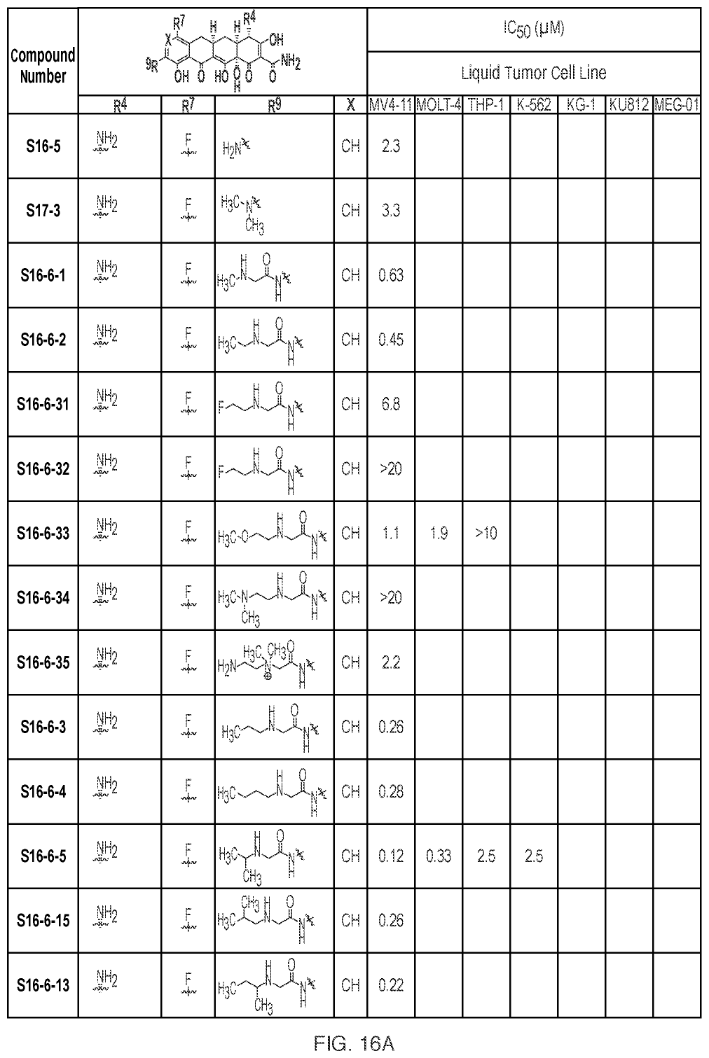

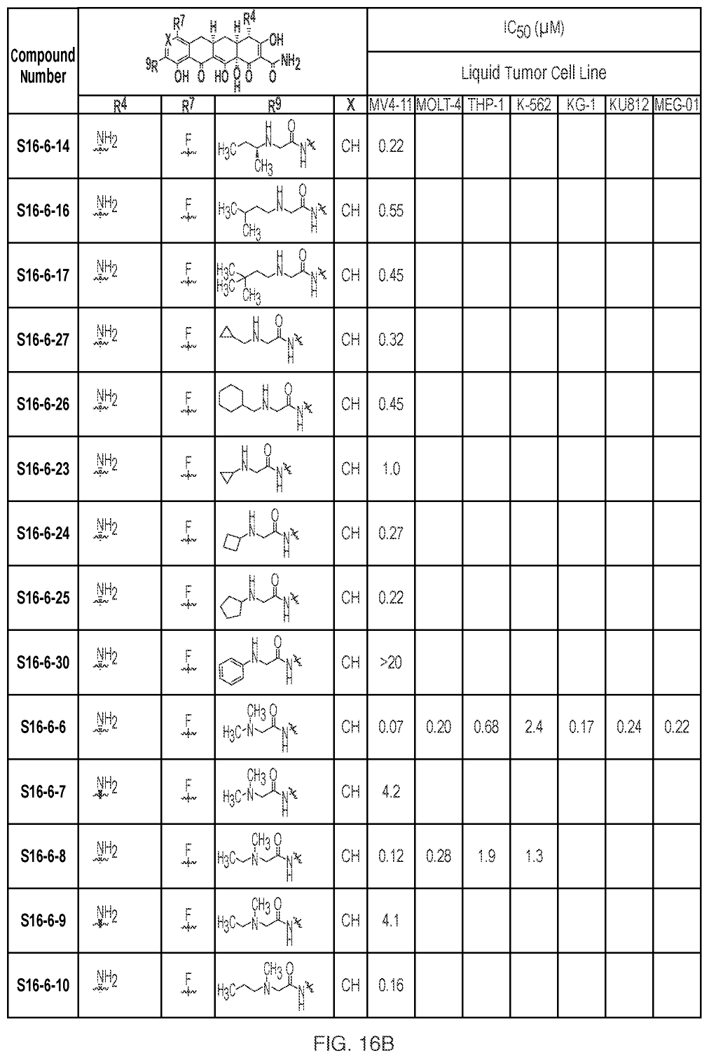

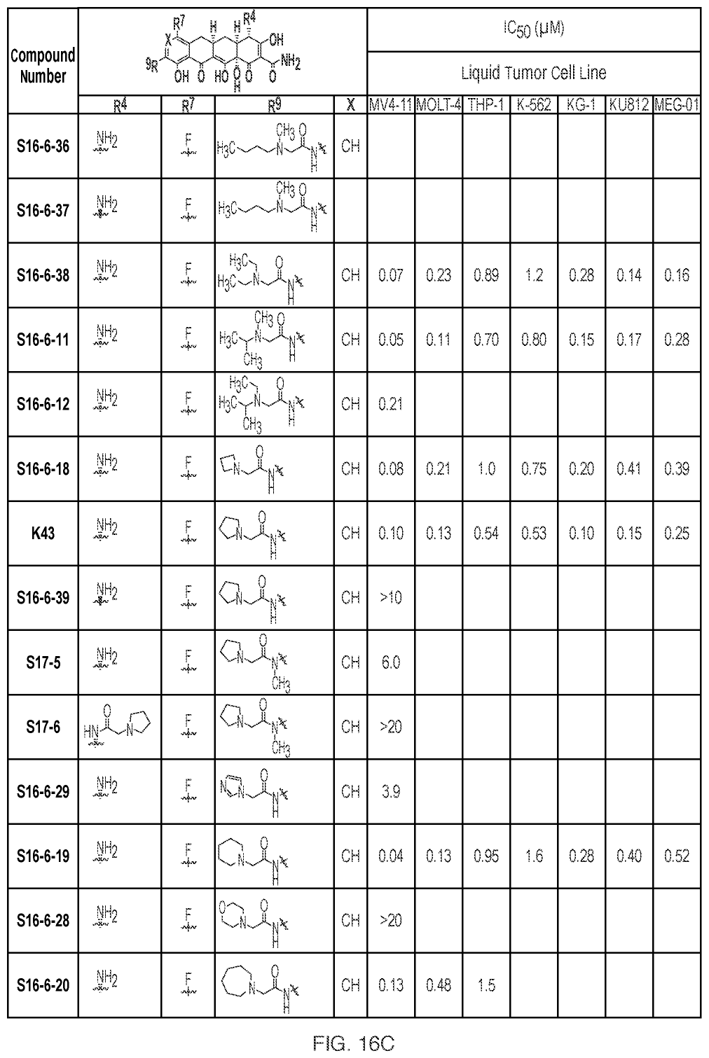

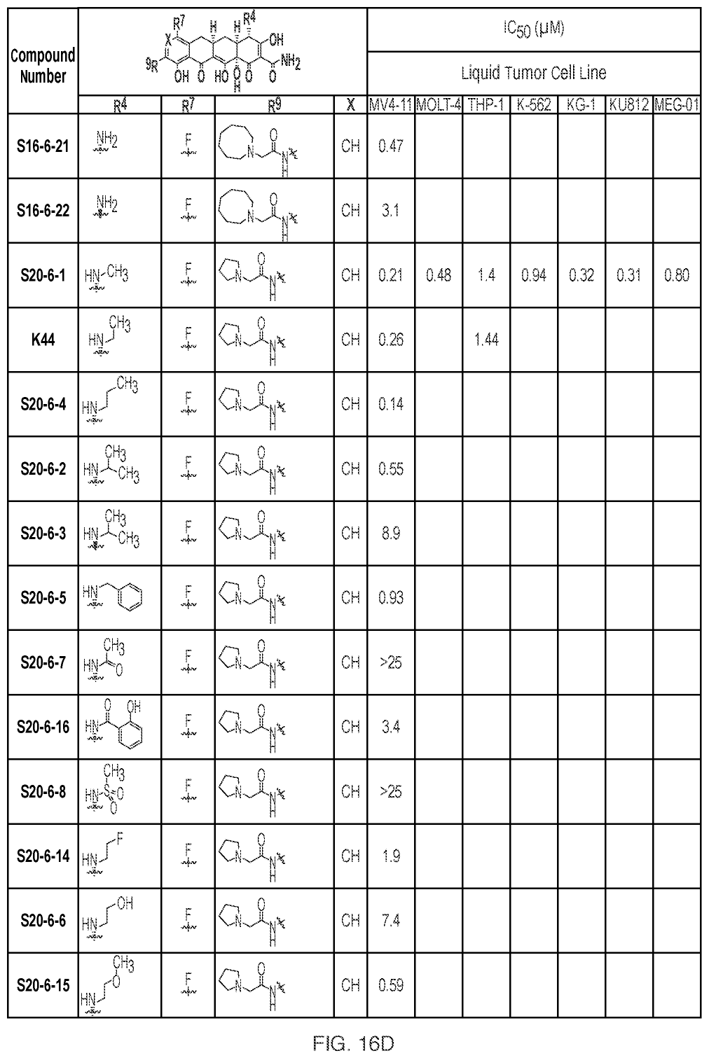

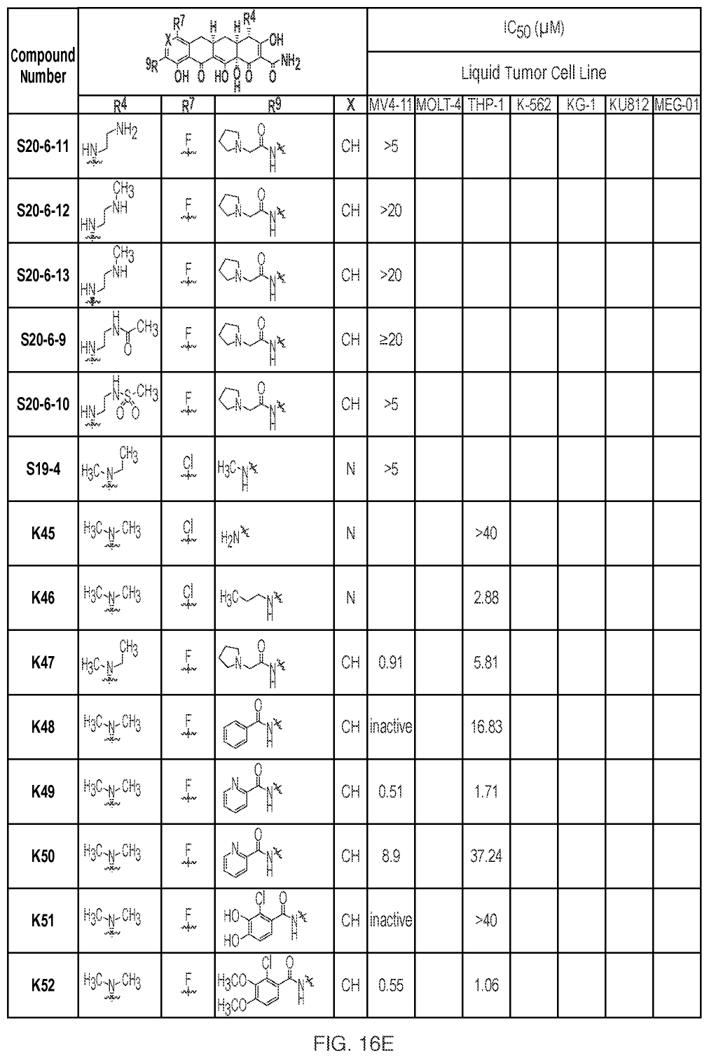

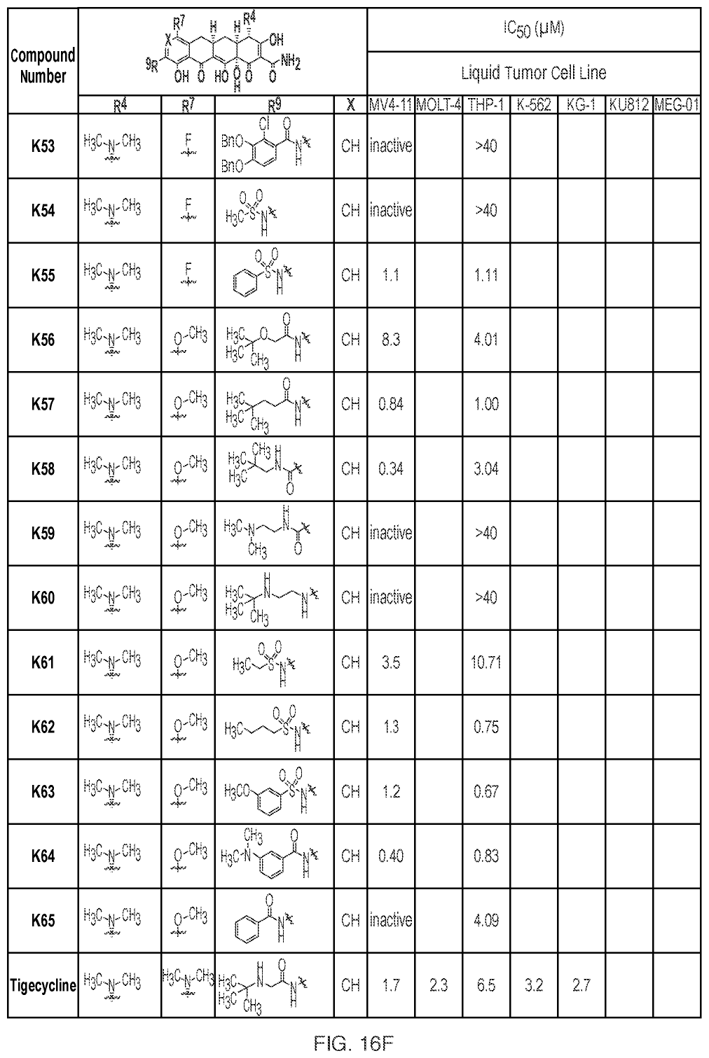

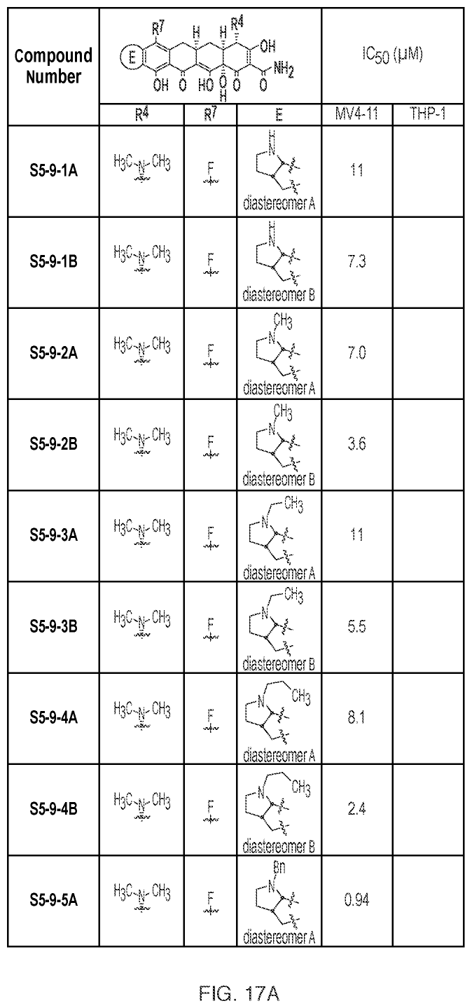

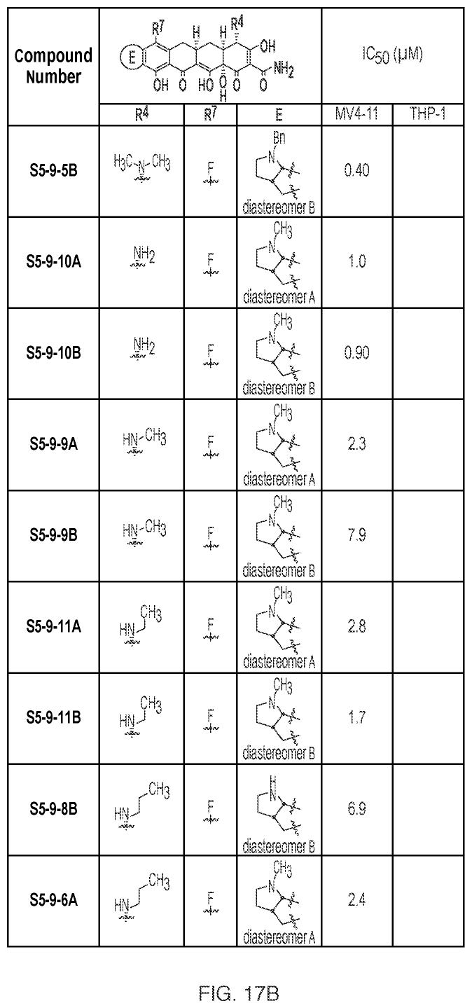

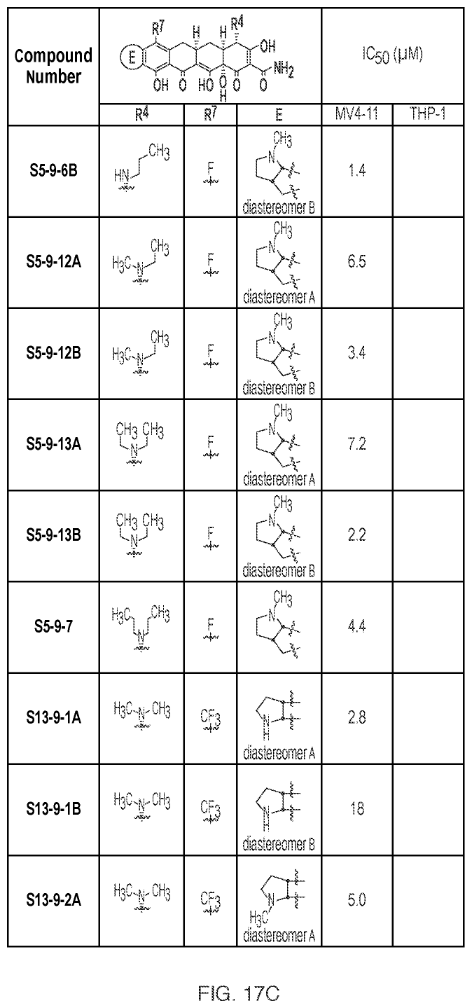

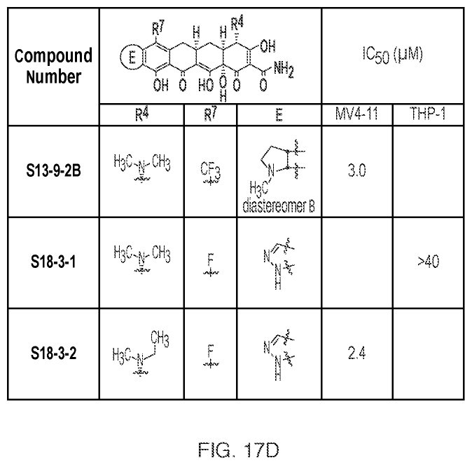

Compounds 1, 2, 3a, 4a and 5 and the Compounds of FIGS. 15A-15M, 16A-16F and 17A-17D were assayed for tumor cell proliferation using AML cancer cell lines THP-1 and MV4-11. The inhibition of cytrochrome-oxidase 1 (COX-1) expression in MV4-11 cells was also measured for Compounds 1, 2, 3a, 4a and 5.

A. THP-1 Anti-Proliferation Assay

The inhibition of eukaryotic cell culture growth was established using THP-1 cells (ATCC Cat. #TIB-202), a human acute monocytic leukemia cell line. These are suspension cells. This cell-based assay for eukaryotic culture growth inhibition was performed in 384-well plate format to determine the in vitro cytotoxicity of the test compounds.

Compounds were solubilized in water. Compounds were diluted 1:2 in assay media and 1:2 serial dilutions were performed in 50:50 media:water mix. The high dose was 40 μM, 10% water final. 5 μL of compound at 5× the final concentration was dispensed to the 384 assay plate. 20 μL of THP-1 cells were added.

Compounds were plated in dose response format (10% water final concentration), followed by the addition of cells. Cells were grown and incubated with compounds in RPMI-1640 medium/pen/strep/L-glutamine/10% FBS/2-mercaptoethanol for 72 hr at 37° C. with 5% CO₂. At the end of the incubation time, cells were assayed for viability using Cell Titer GLO (Promega). Compounds that were considered cytotoxic resulted in a decreased luminescent signal.

B. MV4-11 Anti-Proliferation Assay

MV4-11 cell line (MV-4-11, CRL-9591™) was obtained from American Type Culture Collection (ATCC). Cells were grown in a T-75 flask in RPMI Medium (GIBCO, Catalog No. 11875-093) containing 10% fetal bovine serum (ATCC, Catalog No. 30-2020) and penicillin-streptomycin (ATCC, Catalog No. 30-2300) at 37° C. in a humidified, 5% CO₂ incubator.

50 μL of cells (10,000 cells/well) were plated in a 96-well plate and incubated overnight at 37° C. in a humidified, 5% CO₂ incubator. The next day, 50 μL of medium containing 3-fold serially diluted compounds in duplicate was added to the wells such that the starting concentration of the compound in the first pair of the wells was 10 μM. After 72 hour incubation with the compound, cell viability was measured in a luminometer after the addition of 100 μL/well CellTiterGlo reagent (Promega) as recommended by the manufacturer. The IC₅₀ values for the compounds were calculated using SoftMax software.

C. Anti-Proliferative Activity

As the data in Table 1A shows, compounds 1, 2, 3a, 4a and 5 demonstrate potent anti-proliferative activity with IC₅₀ values of 0.10 to 1.05 μM against two AML cancer cell lines, THP-1 and MV4-11.

TABLE 1A

| | |
|---|---|
| Compound | IC50 (μM) |

| ID | THP-1 | MV4-11 |
|---|---|---|
| 1 | 0.57 | 0.22 |
| 2 | 1.05 | 0.41 |
| 3a | 0.75 | 0.10 |
| 4a | 0.90 | 0.13 |
| 5 | 0.64 | 0.17 |
| Tigecycline | 29.13 | 4.64 |

Further results of the testing of certain compounds described herein in the THP-1 and MV4-11 cell lines are reported in FIGS. 15A-15M, 16A-16F and 17A-17D.

D-1. Anti-Proliferative Activity of Compounds in Additional Cell Lines

Compounds 1, 2, 3a, 4a and 5 as well as certain Compound included in FIGS. 15A-15M, 16A-16F and 17A-17D were tested in the following cell lines: MOLT4 and K562. Compounds 1, 2, 3a, 4a and 5 were also tested in the cell line HL60.

### Cell Lines and Culture:

The MOLT4 cell line (CRL-1582™) and K562 cell line (CCL-243™) were obtained from American Type Culture Collection (ATCC). They were grown in a T-75 flask in RPMI Medium (GIBCO, Catalog No. 11875-093) containing 10% fetal bovine serum (ATCC, Catalog No. 30-2020) and penicillin-streptomycin (ATCC, Catalog No. 30-2300) at 37° C. in a humidified, 5% CO₂ incubator. The HL60 cell line (CCL-240™) was obtained from American Type Culture Collection (ATCC). They were grown in a T-75 flask in DMEM Medium (GIBCO, Catalog No. 11965-092) containing 20% fetal bovine serum (ATCC, Catalog No. 30-2020) and penicillin-streptomycin (ATCC, Catalog No. 30-2300) at 37° C. in a humidified, 5% CO₂ incubator.

### Proliferation Assay:

50 μL of cells (8,500 cells/well) were plated in a 96-well plate and incubated overnight at 37° C. in a humidified, 5% CO₂ incubator. The next day, 50 μL of medium containing 3-fold serially diluted compounds in duplicate was added to the wells such that the starting concentration of the compound in the first pair of the wells was 10 μM. After 72 hour incubation with the compound, cell viability was measured in a luminometer after the addition of 100 μL/well CellTiterGlo reagent (Promega) as recommended by the manufacturer. The IC₅₀ values for the compounds were calculated using SoftMax software.

TABLE 1B

| Compound | IC50 (μM) | | |
|---|---|---|---|
| ID | MOLT4 | K562 | HL60 |
| 1 | 0.19 | 0.43 | 0.36 |
| 2 | 0.31 | 0.48 | Inactive |
| 3a | 0.12 | 0.11 | 0.37 |
| 4a | 0.13 | 0.13 | 0.41 |
| 5 | 0.13 | 0.16 | 0.51 |
| Tigecycline | 5.1 | 7.2 | 15.4 |

Results of the testing of additional compounds described herein in the MOLT4 and K562 cell lines are reported in Tables 15A-15M, 16A-16F and 17A-17D.

D-2. Anti-Proliferative Activity of Compounds in KG-1, KU812 and MEG-01 Cell Lines

Compounds were tested in the following cell lines: KG-1 acute myelogenous leukemis ATCC CCL-246, KU812 Human chronic myelogenous leukemia (CML) ATCC CRL-2099, and MEG-01 Human chronic myelogenous leukemis (CML) ATCC CRL-2021 according to the following conditions and procedures:

Growth medium: RPMI Medium 1640 Gibco #11875-093

### Supplements: Fetal Bovine Serum (FBS) Gibco #10437-028

Complete cell culture medium was prepared by adding 50 mL FBS (final concentration 10%) to each 500 mL bottle of RPMI Medium 1640 (RPMI). Medium was allowed to equilibrate to 37C in a water bath before use.

One millimeter volumes of 2× the initial concentration (20 μM or 100 μM) in complete cell culture medium were prepared for each compound to be tested. 50 μl was added, in triplicate, to lane 2 wells of a 96-well plate and to lane 3 wells containing 50 μl complete cell culture medium as diluent. Two-fold serial dilutions of compounds were continued in lanes 4-10 with 50 μl final volumes. 50 μl medium without compound was added to lane 11, and 100 μl medium was added to lanes 1, 12 and rows A,H in order to prevent or minimize a thermal gradient from forming in the experimental wells.

Cells grown to 1-4×10⁵/mL were centrifuged, re-suspended in fresh medium at 2×10⁵/mL and 50 μl (10,000 cells) was added to each well containing compound (lanes 2-10) and to 6 wells (lane 11) containing medium only. Addition of cells resulted in dilution of compound to the intended 1× concentrations.

Plates were incubated at 37 C. in 5% CO₂ for 72 hours.

After 72 hours incubation, plates were allowed to equilibrate to room temperature for 30 minutes and assayed for cell proliferation using the Promega CellTiter-Glo Kit (Promega #G7572) which indirectly measures ATP. 100 μl of CellTiter-Glo substrate reconstituted with CellTiter-Glo buffer was added to each well containing cells as well as 6 wells containing medium only. Plates were incubated at room temperature, protected from light, for 10 minutes to allow luminescence signal to stabilize. Luminescence was read and recorded in a LUMIstar OPTIMA luminescence microplate reader using MARS Data Analysis Software (BMG LABTECH).

The luminescence values for compound-containing wells (in triplicate) were plotted as the mean % no-compound control vs concentration using Prism GraphPad. IC₅₀s, the concentration at which a compound reduced growth (as measured by ATP) by 50%, were obtained from the graphs.

$$\% no - compound control = \frac{Flourescence value ( cells plus compound ) \times 100}{Flourescence values ( cells , no compound ) , averaged}$$

### Plate Format

| | | | | | | | | | | | |
|---|---|---|---|---|---|---|---|---|---|---|---|
| media | media | media | media | media | media | media | media | media | media | media | media |
| media | TP-compound (10 μM) | 5 | 2.5 | 1.25 | 0.63 | 0.31 | 0.16 | 0.08 | 0.04 | cells | media |
| media | TP-compound (10 μM) | 5 | 2.5 | 1.25 | 0.63 | 0.31 | 0.16 | 0.08 | 0.04 | cells | media |
| media | TP-compound (10 μM) | 5 | 2.5 | 1.25 | 0.63 | 0.31 | 0.16 | 0.08 | 0.04 | cells | media |
| media | Tigecycline (50 μM) | 25 | 12.5 | 6.25 | 3.12 | 1.56 | 0.78 | 0.39 | 0.20 | cells | media |
| media | Tigecycline (50 μM) | 25 | 12.5 | 6.25 | 3.12 | 1.56 | 0.78 | 0.39 | 0.20 | cells | media |
| media | Tigecycline (50 μM) | 25 | 12.5 | 6.25 | 3.12 | 1.56 | 0.78 | 0.39 | 0.20 | cells | media |
| media | media | media | media | media | media | media | media | media | media | media | media |

TP-Compound in the table above, refers to compounds described herein that were being tested.
The results of testing in the KG-1, KU812 and MEG-01 cell lines are reported in Tables 15A-15M, 16A-16F and 17A-17D.

E. Antiproliferative Activity Against 15 AML Ex-Vivo Bone Marrow Sample

Antiproliferative activity of Compound 3a and cytarabine was measured against 15 AML ex-vivo bone marrow samples (including two cytarabine-resistant samples). The assay used was Vivia's Native Environment cell depletion assay. An outline of the study is below:
Five different concentrations of each drug was used as a monotherapy
The incubation time point for measurement was 48 hours post drug exposure
   The results are shown graphically in FIG. 6. Compound 3a provided potent ex vivo activity against the tumor cells from frozen bone marrow of the AML patients. The activity of Compound C₃a was better that cytarabine with stronger potency and higher efficacy. According to the activity profile observed, Compound 3a had a mean EC50 value of 170 nM.

F. MV4-11 Xenografts

The in vivo anti-tumor efficacy of Compounds 3a, 4a and 5 in the subcutaneous MV4-11 leukemia model in CB17 SCID mice was tested.

### Cell Culture:

The MV4-11 cells (ATCC-CRL-9591) were maintained in vitro as a suspension culture at a density of 0.2-1.5×106 cells/ml in RPMI1640 medium supplemented with 10% heat inactivated fetal bovine serum, 100 U/ml penicillin and 100 μg/ml streptomycin at 37° C. in an atmosphere of 5% CO₂ in air. The tumor cells were routinely subcultured twice weekly. The cells growing in an exponential growth phase were harvested and counted for tumor inoculation.

### Animals:

CB17 SCID, female, 6-8 weeks, weighing approximately 18-22 g.

### Tumor Inoculation:

Each mouse was inoculated subcutaneously at the right flank with MV4-11 tumor cells (10×10⁶) in 0.2 ml of PBS (with Matrigel 1:1) for tumor development. The animals were randomized and treatment was started when the average tumor volume reached approximately 150-200 mm³ for the efficacy study. The test article administration and the animal numbers in each group are shown below.

TABLE 1 C
Groups and Treatments

| | | | | Dosin | |
|---|---|---|---|---|---|
| | | | Dose | g | |
| Group | N^{a} | Treatment | (mg/kg) | Route | Schedule |
| 1 | 10 | Vehicle Control | — | I.P. | QD^{b}, 4 days on, |
| | | | | | 6 days off, day 0-14 |
| 2 | 10 | Cytarabine | 100 | I.P. | QD, 5 days on, |
| | | | | | 2 days off, day 0-14 |
| 3 | 10 | Tigecycline | 50 | I.P. | BID^{c}, day 0-14 |
| 4 | 10 | Compound 3a - | 12.5 | I.P. | QD, 4 days on, |
| | | dose 1 | | | 6 days off, day 0-14 |
| 5 | 10 | Compound 3a - | 12.5 | I.P. | QD, 2 days on, |
| | | dose 2 | | | 6 days off, day 0-14 |
| 6 | 10 | Compound 4a - | 12.5 | I.P. | QD, 4 days on, |
| | | dose 1 | | | 6 days off, day 0-14 |
| 7 | 10 | Compound 4a - | 12.5 | I.P. | QD, 2 days on, |
| | | dose 2 | | | 6 days off, day 0-14 |
| 8 | 10 | Compound 5 - | 12.5 | I.P. | QD, 4 days on, |
| | | dose 1 | | | 6 days off, day 0-14 |
| 9 | 10 | Compound 5 - | 12.5 | I.P. | QD, 2 days on, |
| | | dose 2 | | | 6 days off, day 0-14 |

Note:
^{a}N: number of animals per group;
^{b}QD: once per day;
^{c}BID: twice per day. BID dosing is 8 hours apart.

### Endpoints:

The major endpoint monitored was tumor growth delay or cure. Tumor sizes were measured twice weekly in two dimensions using a caliper, and the volume expressed in mm³ using the formula: V=0.5 a×b² where a and b are the long and short diameters of the tumor, respectively. The tumor sizes were then used for the calculations of both T-C and T/C values. T-C is calculated with T as the median time (in days) required for the treatment group tumors to reach a predetermined size (e.g., 1,000 mm³), and C is the median time (in days) for the control group tumors to reach the same size. The T/C value (in percent) is an indication of antitumor effectiveness, T and C are the mean volume of the treated and control groups, respectively, on a given day.

TGI was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

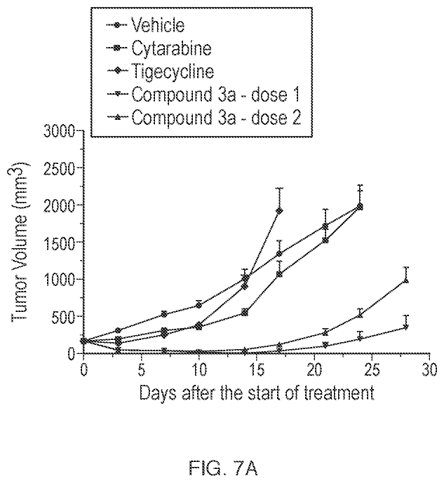

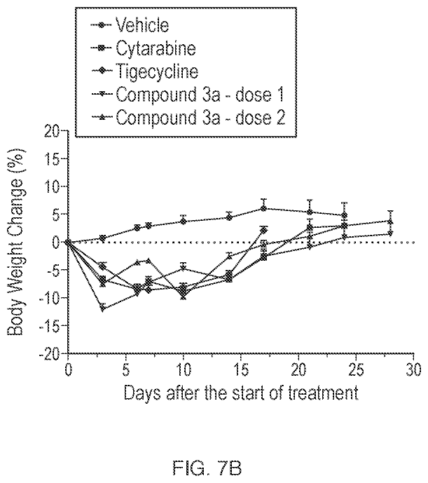

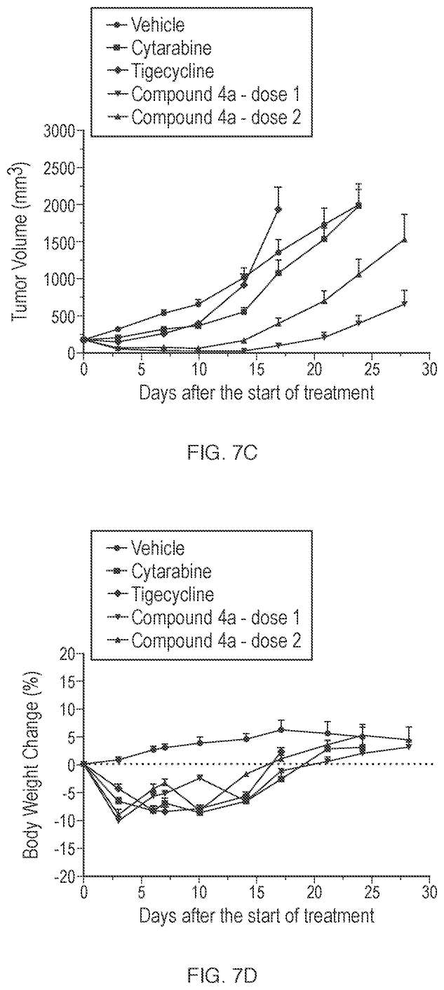

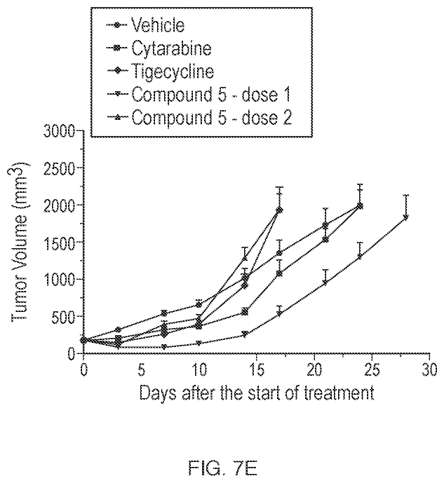

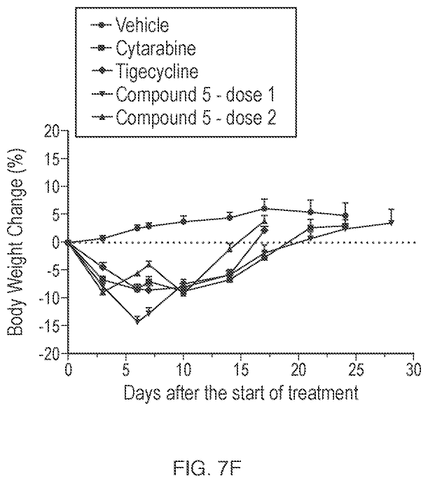

The results of Tumor Volume versus Time and Body Weight versus Days after Start of Treatment are shown in FIGS. 7A-7F. As can be see in FIG. 7, all animals treated with Compound 3a achieved ≥70% tumor shrinkage. Cytarabine (standard of care) and tigecycline dosed at maximum tolerated does (MTD) only demonstrated modest effect-no tumor response in either group.

G. Effect of Compound 3a on Rat Heart Mitochondrial Protein Synthesis

The effect of Compound 3a on mitochondrial protein synthesis was determined using an intact isolated rat heart mitochondrial protein synthesis assay previously described [See, 1, 2 below]. Intact, highly coupled mitochondria isolated from normal rat hearts were incubated in an incubation medium containing [S³⁵]-methionine. The compounds were diluted to generate a final dose-response curve from 0.15 to 40 μM. The rate of incorporation of [S³⁵]-methionine into protein was measured at 20, 40, and 60 minutes of incubation for each sample using a filter paper disc assay and expressed as pmol methionine incorporated per mg mitochondrial protein as described [1, 2, 3 below]. The time course data for control and all drug concentrations were nearly linear. The slope of each time-course data plot is calculated as the least squares best fit line through zero and the three time points for each sample. The rate of protein synthesis varies modestly with each mitochondrial preparation (mean and SEM=20.3+/−2.4 pm/mg protein). To normalize for this variability, the rates were expressed as a percent of the rate of the control line for each preparation of mitochondria.

Each experiment was repeated three times.

Dose-response curves were obtained by expressing the percent of control obtained for each concentration of Compound 3a against the concentration of Compound 3a. The dose-response curves for all three experiments were plotted together and fit using the equation y=ab/(b+x) (Sigma Plot 10.0) and the half-maximal inhibitory concentration (IC₅₀) is reported for each drug.

### Summary of Results:

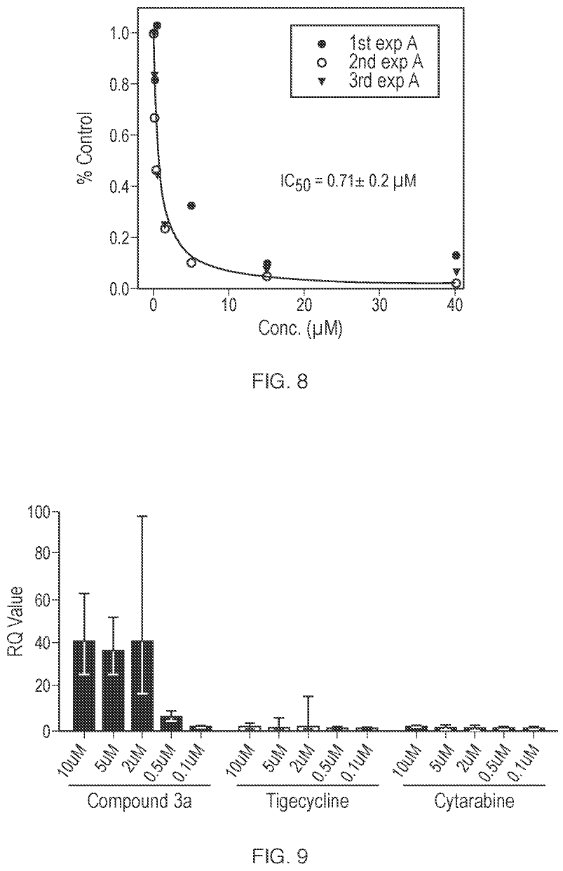

FIG. 8 shows the dose-response results for Compound 3a. The dose-response curve IC₅₀ was 0.7 μM. As such the data represents both the ability of Compound 3a to cross the mitochondrial membranes and to inhibit mitochondrial translation.
1. McKee, E. E., Ferguson, M., Bentlye, At. T. and Marks, T. A. (2006) Inhibition of mammalian mitochondrial protein synthesis by oxazolidinones. Antimicrob Agents Cehmother 50, 2042-2049.
2. McKee, E. E., Grier, B. L., Thompson, G. S. and McCourt, J. D. (1990) Isolaton and incubation conditions to study heart mitochrondrial protein synthesis. Am J Physiol 258, E492-502.
3. Flanagan, S., McKee, E. e., Das, D., Tulkens, P. M., Hosako, H., Fiedler-Kelly, J., Passarell, J., Rodovsky, A., and Prokocimer, P. Nonclinical and pharmacokinetic assessments to evaluate the potential of tedizolid and linezolid to affect mitochondrial function (2014) Antimicrobial Agents and Chemo 59: 178-185, doi 10.1128/AAC03684, PMID 25331703.

H. COX1 and COX4 Protein Levels in MV4-11 Cells

### Materials:

1) MV4-11 cell line: MV411 cell line (MV-4-11, CRL-9591™) was obtained from American Type Culture Collection (ATCC).
2) Antibodies: The following antibodies were purchased as shown below in Table 2, and dilutions were used in the western blot analyses as recommended by the manufacturer.

TABLE 2

| | | Catalogue |
|---|---|---|
| Antibody | Company | Number |
| Anti- human | BosterBio | PA1317 |
| C0X1 in rabbit | | |
| Anti- human | Santa Cruz | SC376831 |
| COX4 in mouse | Laboratories | |
| HRP-conjugated | Bio-Rad | 170-5047 |
| anti-mouse | | |
| HRP-conjugated | Bio-Rad | 170-5046 |
| anti-rabbit | | |
| HRP-anti human | Cell Signal | 12262S |
| β-actin | Technologies | |

### Methods:

1) Cell Lines and culture conditions: MV4-11 cells were grown in a T-75 flask in RPMI Medium (GIBCO, Catalog No. 11875-093) containing 10% fetal bovine serum (ATCC, Catalog No. 30-2020) and penicillin-streptomycin (ATCC, Catalog No. 30-2300) at 37° C. in a humidified, 5% CO₂ incubator.
2) Compound treatment: Two mL of cells (500,000 cells) were plated in each well of a 6-well plate and incubated at 37° C. in a humidified, 5% CO₂ incubator. The next day, 2.5 μL, 6.25 μL, 12.5 μL, 25 μL and 50 μL of 400 μM compound was added to each well. These additions resulted in 0.5 μM, 1.25 μM, 2.5 μM, 5 μM, and 10 μM final concentrations of the compounds. One well did not receive any compound, which serves as untreated control. After 18 hours of incubation with the compounds, cells were collected by centrifugation at 2000 g for one minute and washed with one mL of PBS. The cell pellet was lysed in 50 μL of lysis buffer, and stored at −20° C. until further use.
3) Protein Estimation: Cell lysates were spun at 12,000 rpm for one minute, and 3 μL of the supernatant was used to check the protein concentration using the Coomassie blue reagent following the recommended protocol. For electrophoresis, equal amount of protein extract was used for each compound. The amount of protein extract loaded varied from 7.5 to 15 μg per sample for different compounds.
4) Western Blotting:

### Sample Preparation

x μL of cell lysate (adjusted volumes for equal protein concentrations)

### 0.1 μL μTT (1M)

15-x μL of lysis buffer
5 μL of 4× Laemmle's sample buffer
The samples were heated at 95° C. for 5 minutes.

### Gel Electrophoresis:

a) NUPAGE 4-12% Bis-Tris Gel (Novex, Catalog no. NPO322BOX) was assembled in a XCell II Blot module (Invitrogen, Catalog no. E19051) and running buffer was added.
b) 20 μL of samples and 5 μL of pre-stained molecular weight markers were separately loaded in the wells.
c) Gels were run at 150V for about 1.5 hours until the blue dye reached the bottom.
   Protein Transfer from the Gel to the Nitro Cellulose Membrane:
a) After the run, the gel was removed and protein transfer was performed using iBlot (Invitrogen, Catalog No. IB301002) as per manufacturer's recommendations.

### Primary Antibody Incubation:

a) The nitrocellulose membrane was removed, and placed in 20 mL of blocking solution (5% TBST containing 5% milk) at room temperature for 1 hr.
b) The blot was washed 3 times for 5 min with TBST.
c) The blot was incubated overnight in 15 mL of TBST containing 0.5% BSA, 0.02% sodium azide and 15 μL of the anti-COX1 or 37.5 μL anti-COX4 antibody at room temperature.
d) The blot was washed 3 times for 5 min each with TBST.
   Secondary Antibody incubation:
a) The blot was incubated in 15 mL of TBST containing 0.5% BSA and 1.5 μL of the HRP-conjugated secondary anti-rabbit antibody (for COX1 blots) or anti-mouse antibody (for COX4 blots) solution for 1 h at room temperature.
b) The blot was washed for 3 times for 5 min each with TBST.

### Imaging

a) The blots were placed on plastic wrap.
b) A working solution of the substrate was prepared by mixing Substrate A and Substrate B in a 40:1 ratio (Thermo Scientific, Catalog No. 32132) and one mL/blot was added such that the blot is evenly covered with the substrate solution. It was incubated at room temperature for 4 minutes.
c) The blot was covered with another layer of plastic wrap, placed in a cassette and exposed to an X-ray film in a safe lit dark-room.
d) After one minute, the film was removed from the cassette, and developed.

### Probing for Beta-Actin:

a) To monitor beta-actin levels, the blots were washed three times for 5 minutes each in TBST, and incubated in 15 mL of TBST containing 0.5% BSA and 3 μL of the HRP-conjugated beta-actin antibody for one hour at room temperature.
b) The blot was washed three times for 5 minutes each with 15 mL of TBST, and imaged as described above.

### Reagents and Buffers:

1× Cell lysis/Protein Extraction Reagent (Cell Signal Technology, Cat. no: 9803)
20 mM Tris-HCl (pH 7.5)
150 mM NaCl
1 mM Na₂EDTA
1 mM EGTA
10% Triton
2.5 mM sodium pyrophosphate
1 mM b-glycerophosphate
1 mM Na₃VO₄
1 μg/mL leupeptin
Protease inhibitors (Roche, Catalog no. 11873580001)
Protein Estimation: Coomassie protein assay reagent (ThermoScientific, Cat. No. 1856209)
Sample Loading Buffer for Electrophoresis
4× Laemmli Sample buffer (Novex, Catalog No. NP0007)
Electrophoresis Running Buffer
NuPAGE MOPS/SDS running buffer (Novex, Catalog no. NP0001)
Wash Buffer for Western blots
Tris-buffered saline with Tween 20 (TBST buffer)
20 mM Tris-HCl (pH 7.5)
150 mM NaCl
0.1% Tween 20
Blocking buffer for Western blots
5% Non-fat dry milk in TBST
Signal detection kit: Pierce ECL Plus Substrate (Thermoscientific, Catalog no. 32132)
Electrophoresis gel: NuPAGE 4-12% Bis-Tris Gel (Novex, Catalog no. NPO322BOX)

5) Effects on COX1 and COX4 Protein Levels in MV4-11 Cells

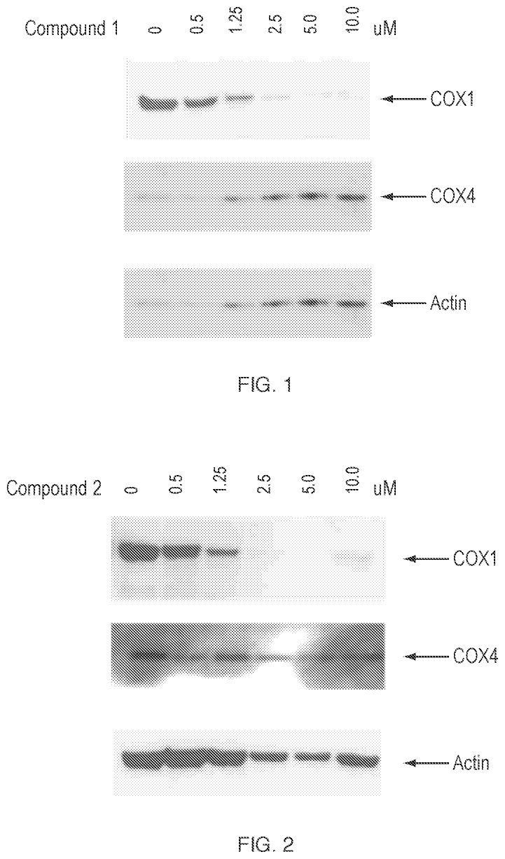

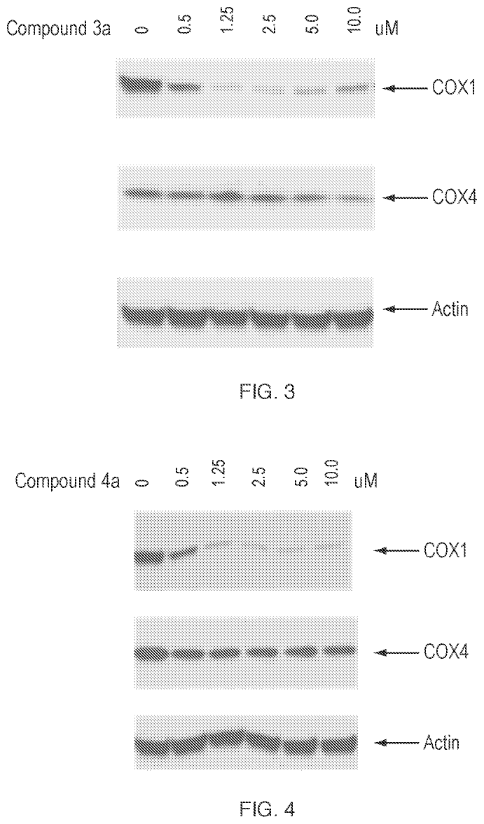

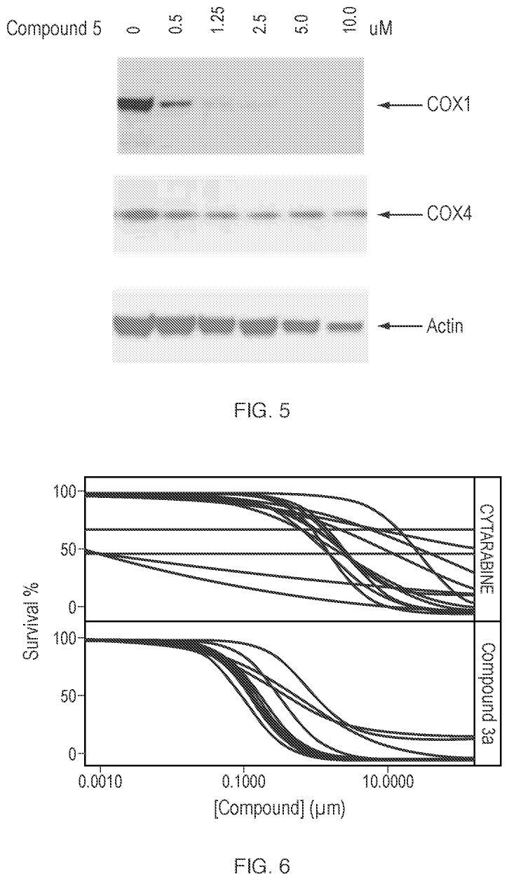

As shown in the western blots in FIGS. 1-5, all five compounds reduced the expression of mitochondrially translated COX1 protein with increasing compound concentrations, while COX4 and actin levels remained relatively unchanged.

Gene Expression Changes in MV4-11 when Treated with Compound 3a, Tigecycline and Cytarabine

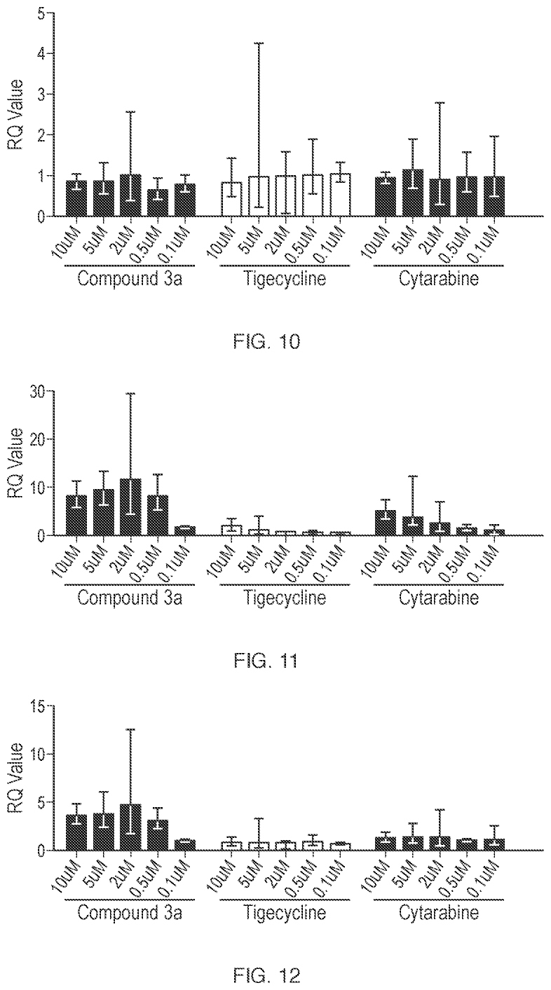

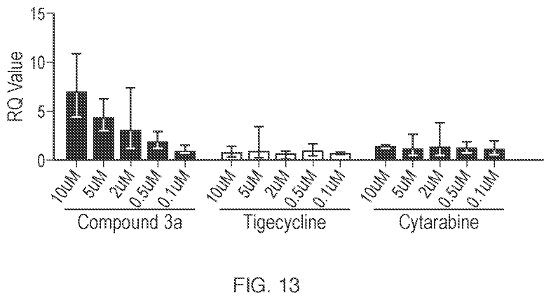

MV411 cells were plated at about 1×10⁵/ml in 24 well plates, grown overnight at 37° C./5% CO₂ in RPMI 1640/10% FBS. Wells were harvested for RNA preparation using Qiagen RNeasy kit. Samples prepped in triplicate, cDNA made using about 100 ng total input RNA. qPCR assays run on Applicaed Biosystems Step One Plus instrument using commercially available primer/probe designs. The results for MV411 MT-COX1 (Cytochrome oxidase subunit 1, expressed in mitochrondria) expression are shown in FIG. 9. The results for MV411 COX-IV expression (Cytochrome oxidase subunit 4, expressed in nucleus) are shown in FIG. 10. The results for MV411 PIG3 expression (TP₅₃I₃-a p53 responsive protein, expression induced in response to p53 activation, role associated with response to oxidative stress) are shown in FIG. 11. The results for MV411 BAX expression (pro-apoptotic protein expression induce by p53 activation, forms a heterodimer with BCL2 to induce apoptosis) are shown in FIG. 12. The results of CDKN2A expression (also known as p14^{ARF} or ARF-nuclear gene, translation regulated by cMyc, functions to stabilize/activate p53 by binding and sequestering Mdm2) are shown in FIG. 13.

### Example 3: Synthesis of Example Compounds

The following abbreviations are used in the paragraphs below:
Ac acetyl
aq aqueous
9-BBN 9-borabicyclo[3.3.1]nonane
BHT t-butyl hydroxyl toluene
Bn benzyl
Boc tert-butoxycarbonyl
Bu butyl
dba dibenzylideneacetone
DCE 1,2-dichloroethane
DCM dichloromethane
DEM diethoxymethane
DIBAL-H diisobutylaluminum hydride
DIEA diisopropylethylamine
DMAP 4-(dimethylamino)pyridine
DME dimethoxyethane
DMF N,N-dimethylformamide
DMPU 1,3-dimethyl-3,4,5,6-tetrahydro-2(1H)-pyrimidone
DMSO dimethylsulfoxide
DPPB 1,4-bis(diphenylphosphinebutane)
ESI ESI ionization
Et ethyl
eq equivalent
h hour
HPLC high performance liquid chromatography
i iso
IBX 2-iodoxybenzoic acid
LDA lithium diisopropylamide
LHMDS lithium bis(trimethylsilyl)amide
M-D Michael-Dieckmann annulation
MHz mega hertz
Ms methylsulfonyl
MS mass spectrometry
MTBE methyl t-butyl ether
m/z mass/charge ratio
MW molecular weight
NCS N-chlorosuccinimide
NDMBA 1,3-dimethylbarbituric acid
NMO N-methylmorpholine N-oxide
NMR nuclear magnetic resonance spectrometry
Ph phenyl
Pr propyl
s secondary
t tertiary
TBAF tetrabutylammonium fluoride
TEA triethylamine
Tf trifluoromethanesulfonyl
TFA trifluoroacetic acid
TFAA trifluoroacetic anhydride
THF tetrahydrofuran
TLC thin layer chromatography
TMEDA N,N,N′N′-tetramethylethylenediamine
TMP 2,2,6,6-tetramethylpiperidine
STAB sodium triacetoxyborohydride

Compounds referred herein as “K-number” (e.g., K1, K2, K43, K44, etc.), have been prepared according to the procedures described in Tables 3A and 3B, below:

TABLE 3A

| | 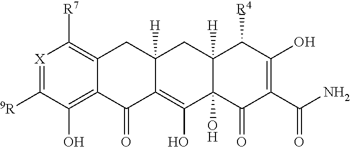 | | | | |
|---|---|---|---|---|---|
| Compound | | | | | Synthetic |
| Number | R⁴ | R⁷ | R⁹ | X | Procedure |
| K43 | 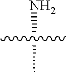 | 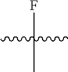 [SMILES: *F] | 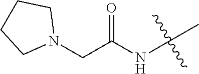 [SMILES: *NC(=O)CN1CCCC1] | CH | See Footnote 1 |
| K44 | 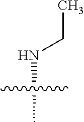 | 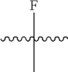 [SMILES: *F] | 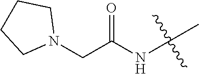 [SMILES: *NC(=O)CN1CCCC1] | CH | See Footnote 1 |
| K45 | 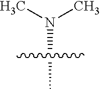 | 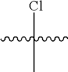 [SMILES: *Cl] | 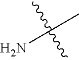 [SMILES: *N] | N | See Footnote 3 |
| K46 | 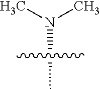 | 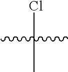 [SMILES: *Cl] | 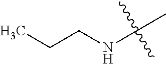 [SMILES: *NCCC] | N | See Footnote 3 |
| K47 | 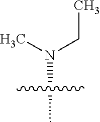 | 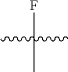 [SMILES: *F] | 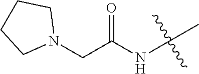 [SMILES: *NC(=O)CN1CCCC1] | CH | See Footnote 1 |
| K48 | 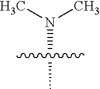 | 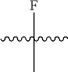 [SMILES: *F] | 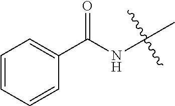 [SMILES: *NC(=O)c1ccccc1] | CH | See Footnote 4 |
| K49 | 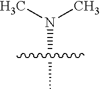 | 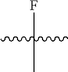 [SMILES: *F] | 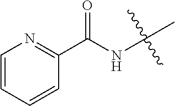 [SMILES: *NC(=O)c1ccccn1] | CH | See Footnote 4 |
| K50 | 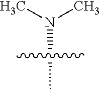 | 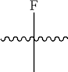 [SMILES: *F] | 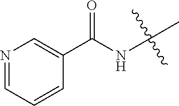 [SMILES: *NC(=O)c1cccnc1] | CH | See Footnote 4 |
| K51 | 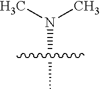 | 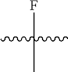 [SMILES: *F] | 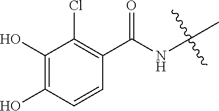 [SMILES: *NC(=O)c1ccc(O)c(O)c1Cl] | CH | See Footnote 4 |
| K52 | 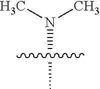 | 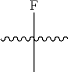 [SMILES: *F] | 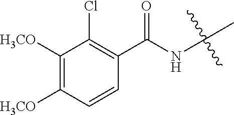 [SMILES: *NC(=O)c1ccc(OC)c(OC)c1Cl] | CH | See Footnote 4 |
| K53 | 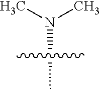 | 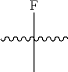 [SMILES: *F] | 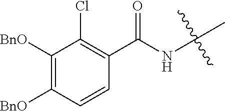 [SMILES: *NC(=O)c1ccc(OCc2ccccc2)c(OCc2ccccc2)c1Cl] | CH | See Footnote 4 |
| K54 | 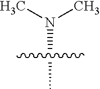 | 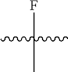 [SMILES: *F] | 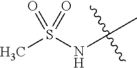 [SMILES: *NS(C)(=O)=O] | CH | See Footnote 4 |
| K55 | 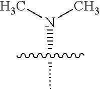 | 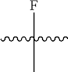 [SMILES: *F] | 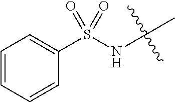 [SMILES: *NS(=O)(=O)c1ccccc1] | CH | See Footnote 4 |
| K56 | 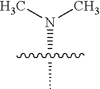 | 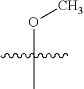 [SMILES: *OC] | 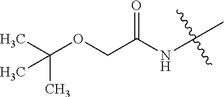 [SMILES: *NC(=O)COC(C)(C)C] | CH | See Footnote 3 |
| K57 | 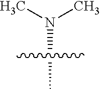 | 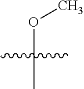 [SMILES: *OC] | 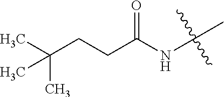 [SMILES: *NC(=O)CCC(C)(C)C] | CH | See Footnote 3 |
| K58 | 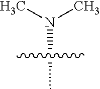 | 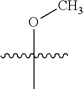 [SMILES: *OC] | 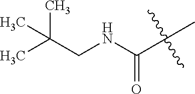 [SMILES: *C(=O)NCC(C)(C)C] | CH | See Footnote 3 |
| K59 | 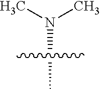 | 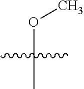 [SMILES: *OC] | 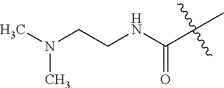 [SMILES: *C(=O)NCCN(C)C] | CH | See Footnote 3 |
| K60 | 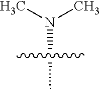 | 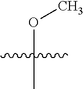 [SMILES: *OC] | 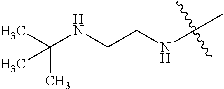 [SMILES: *NCCNC(C)(C)C] | CH | See Footnote 3 |
| K61 | 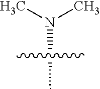 | 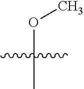 [SMILES: *OC] | 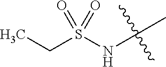 [SMILES: *NS(=O)(=O)CC] | CH | See Footnote 3 |
| K62 | 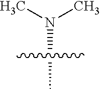 | 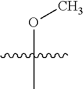 [SMILES: *OC] | 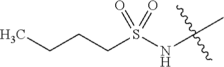 [SMILES: *NS(=O)(=O)CCCC] | CH | See Footnote 3 |
| K63 | 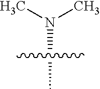 | 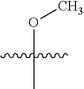 [SMILES: *OC] | 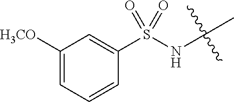 [SMILES: *NS(=O)(=O)c1cccc(OC)c1] | CH | See Footnote 3 |
| K64 | 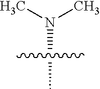 | 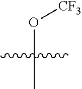 [SMILES: *OC(F)(F)F] | 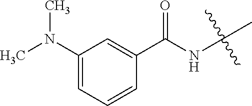 [SMILES: *NC(=O)c1cccc(N(C)C)c1] | CH | See Footnote 3 |
| K65 | 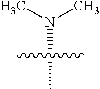 | 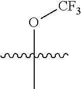 [SMILES: *OC(F)(F)F] | 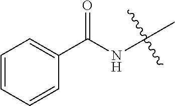 [SMILES: *NC(=O)c1ccccc1] | CH | See Footnote 3 |

¹Compound made in accordance with procedures described in U.S. Pat. No. 9,573,895B2 the entire content of which is hereby incorporated by reference.
²Compound made in accordance with procedures described in U.S. Pat. No. 9,315,451B2 the entire content of which is hereby incorporated by reference.
³Compound made in accordance with procedures described in U.S. Pat. No. 9,624,166B2 the entire content of which is hereby incorporated by reference.
⁴Compound made in accordance with procedures described in U.S. Pat. No. 8,906,887B2 the entire content of which is hereby incorporated by reference.

TABLE 3B

| | 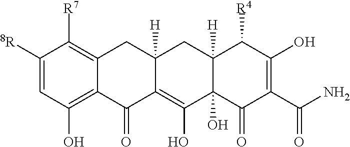 | | | |
|---|---|---|---|---|
| Compound | | | | Synthetic |
| No. | R⁴ | R⁷ | R⁸ | Method |
| K1 | 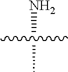 | 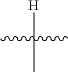 [SMILES: *[H]] | 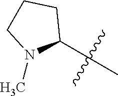 [SMILES: *[C@@H]1CCCN1C] | See Footnote 5 |
| K2 | 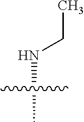 | 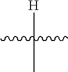 [SMILES: *[H]] | 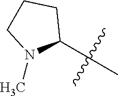 [SMILES: *[C@@H]1CCCN1C] | See Footnote 5 |
| K3 | 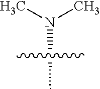 | 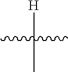 [SMILES: *[H]] | 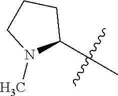 [SMILES: *[C@@H]1CCCN1C] | See Footnote 6 |
| K4 | 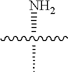 | 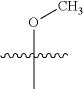 [SMILES: *OC] | 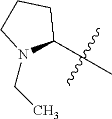 [SMILES: *[C@@H]1CCCN1CC] | See Footnote 5 |
| K5 | 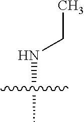 | 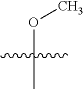 [SMILES: *OC] | 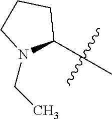 [SMILES: *[C@@H]1CCCN1CC] | See Footnote 5 |
| K6 | 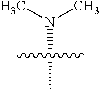 | 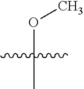 [SMILES: *OC] | 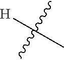 [SMILES: *[H]] | See Footnote 7 |
| K7 | 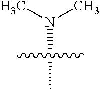 | 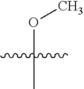 [SMILES: *OC] | 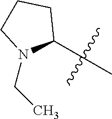 [SMILES: *[C@@H]1CCCN1CC] | See Footnote 6 |
| K8 | 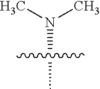 | 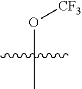 [SMILES: *OC(F)(F)F] | 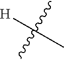 [SMILES: *[H]] | See Footnote 7 |
| K9 | 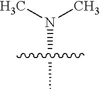 | 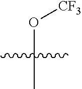 [SMILES: *OC(F)(F)F] | 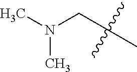 [SMILES: *CN(C)C] | See Footnote 6 |
| K10 | 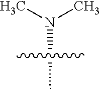 | 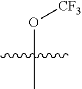 [SMILES: *OC(F)(F)F] | 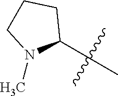 [SMILES: *[C@@H]1CCCN1C] | See Footnote 6 |
| K11 | 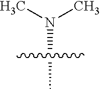 | 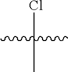 [SMILES: *Cl] | 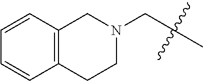 [SMILES: *CN1CCc2ccccc2C1] | See Footnote 6 |
| K12 | 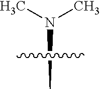 | 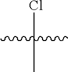 [SMILES: *Cl] | 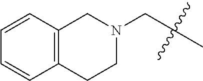 [SMILES: *CN1CCc2ccccc2C1] | See Footnote 6 |
| K13 | 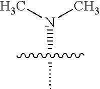 | 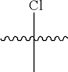 [SMILES: *Cl] | 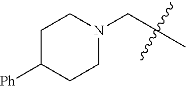 [SMILES: *CN1CCC(c2ccccc2)CC1] | See Footnote 6 |
| K14 | 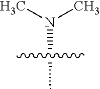 | 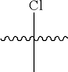 [SMILES: *Cl] | 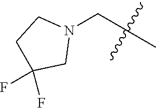 [SMILES: *CN1CCC(F)(F)C1] | See Footnote 6 |
| K15 | 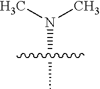 | 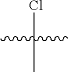 [SMILES: *Cl] | 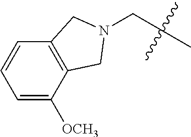 [SMILES: *CN1Cc2cccc(OC)c2C1] | See Footnote 6 |
| K16 | 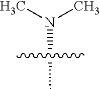 | 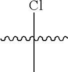 [SMILES: *Cl] | 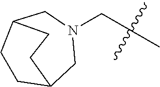 [SMILES: *CN1CC2CCC(CC2)C1] | See Footnote 6 |
| K17 | 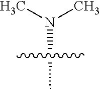 | 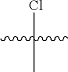 [SMILES: *Cl] | 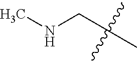 [SMILES: *CNC] | See Footnote 6 |
| K18 | 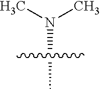 | 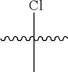 [SMILES: *Cl] | 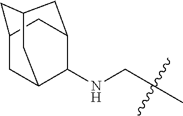 [SMILES: *CNC1C2CC3CC(C2)CC1C3] | See Footnote 6 |
| K19 | 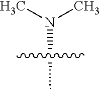 | 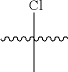 [SMILES: *Cl] | 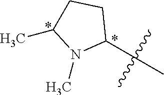 [SMILES: *C1CCC(C)N1C] | See Footnote 6 |
| K20 | 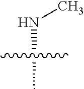 | 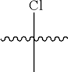 [SMILES: *Cl] | 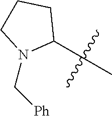 [SMILES: *C1CCCN1Cc1ccccc1] | See Footnote 5 |
| K21 | 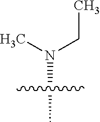 | 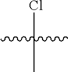 [SMILES: *Cl] | 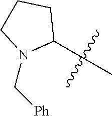 [SMILES: *C1CCCN1Cc1ccccc1] | See Footnote 5 |
| K22 | 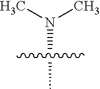 | 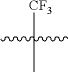 [SMILES: *C(F)(F)F] | 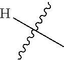 [SMILES: *[H]] | See Footnote 7 |
| K23 | 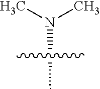 | 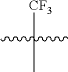 [SMILES: *C(F)(F)F] | 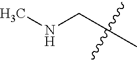 [SMILES: *CNC] | See Footnote 6 |
| K24 | 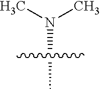 | 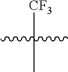 [SMILES: *C(F)(F)F] | 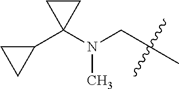 [SMILES: *CN(C)C1(C2CC2)CC1] | See Footnote 6 |
| K25 | 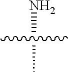 | 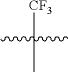 [SMILES: *C(F)(F)F] | 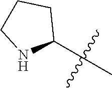 [SMILES: *[C@@H]1CCCN1] | See Footnote 5 |
| K26 | 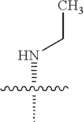 | 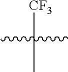 [SMILES: *C(F)(F)F] | 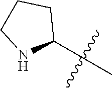 [SMILES: *[C@@H]1CCCN1] | See Footnote 5 |
| K27 | 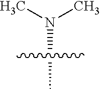 | 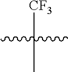 [SMILES: *C(F)(F)F] | 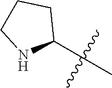 [SMILES: *[C@@H]1CCCN1] | See Footnote 6 |
| K28 | 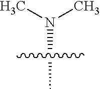 | 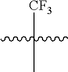 [SMILES: *C(F)(F)F] | 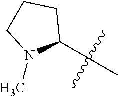 [SMILES: *[C@@H]1CCCN1C] | See Footnote 6 |
| K29 | 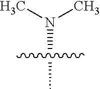 | 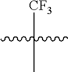 [SMILES: *C(F)(F)F] | 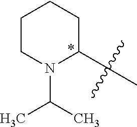 [SMILES: *C1CCCCN1C(C)C] | See Footnote 6 |
| K30 | 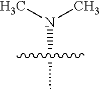 | 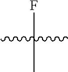 [SMILES: *F] | 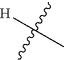 [SMILES: *[H]] | See Footnote 8 |
| K31 | 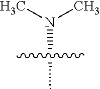 | 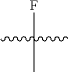 [SMILES: *F] | 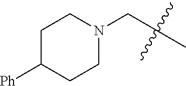 [SMILES: *CN1CCC(c2ccccc2)CC1] | See Footnote 6 |
| K32 | 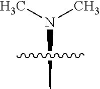 | 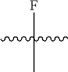 [SMILES: *F] | 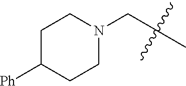 [SMILES: *CN1CCC(c2ccccc2)CC1] | See Footnote 6 |
| K33 | 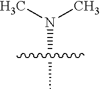 | 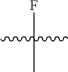 [SMILES: *F] | 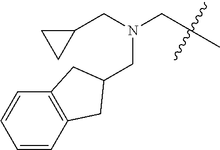 [SMILES: *CN(CC1CC1)CC1Cc2ccccc2C1] | See Footnote 6 |
| K34 | 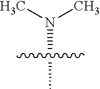 | 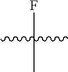 [SMILES: *F] | 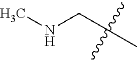 [SMILES: *CNC] | See Footnote 6 |
| K35 | 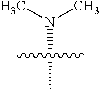 | 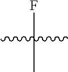 [SMILES: *F] | 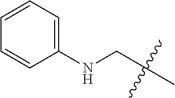 [SMILES: *CNc1ccccc1] | See Footnote 6 |
| K36 | 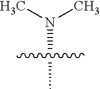 | 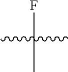 [SMILES: *F] | 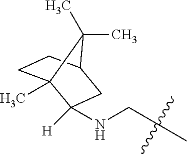 [SMILES: *CNC1CC2CCC1(C)C2(C)C] | See Footnote 6 |
| K37 | 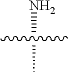 | 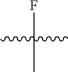 [SMILES: *F] | 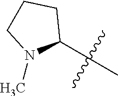 [SMILES: *[C@@H]1CCCN1C] | See Footnote 5 |
| K38 | 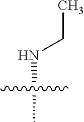 | 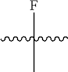 [SMILES: *F] | 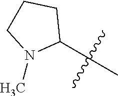 [SMILES: *C1CCCN1C] | See Footnote 5 |
| K39 | 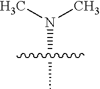 | 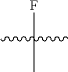 [SMILES: *F] | 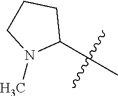 [SMILES: *C1CCCN1C] | See Footnote 6 |
| K40 | 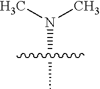 | 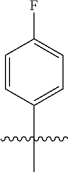 [SMILES: *c1ccc(F)cc1] | 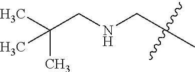 [SMILES: *CNCC(C)(C)C] | See Footnote 6 |
| K41 | 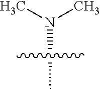 | 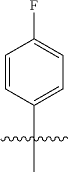 [SMILES: *c1ccc(F)cc1] | 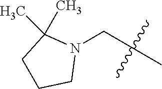 [SMILES: *CN1CCCC1(C)C] | See Footnote 6 |
| K42 | 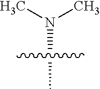 | 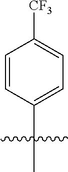 [SMILES: *c1ccc(C(F)(F)F)cc1] | 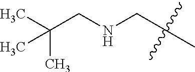 [SMILES: *CNCC(C)(C)C] | See Footnote 6 |

⁵Compound made in accordance with procedures described in U.S. Pat. No. 9,573,895B2 the entire content of which is hereby incorporated by reference.
⁶Compound made in accordance with procedures described in U.S. Pat. No. 9,315,451 the entire content of which is hereby incorporated by reference.
⁷Compound made in accordance with procedures described in U.S. Pat. No. 9,624,166 the entire content of which is hereby incorporated by reference.
⁸Compound made in accordance with procedures described in U.S. Pat. No. 8,906,887 the entire content of which is hereby incorporated by reference.

Further example compounds disclosed herein have been prepared according to Schemes 1 through 21, described below.

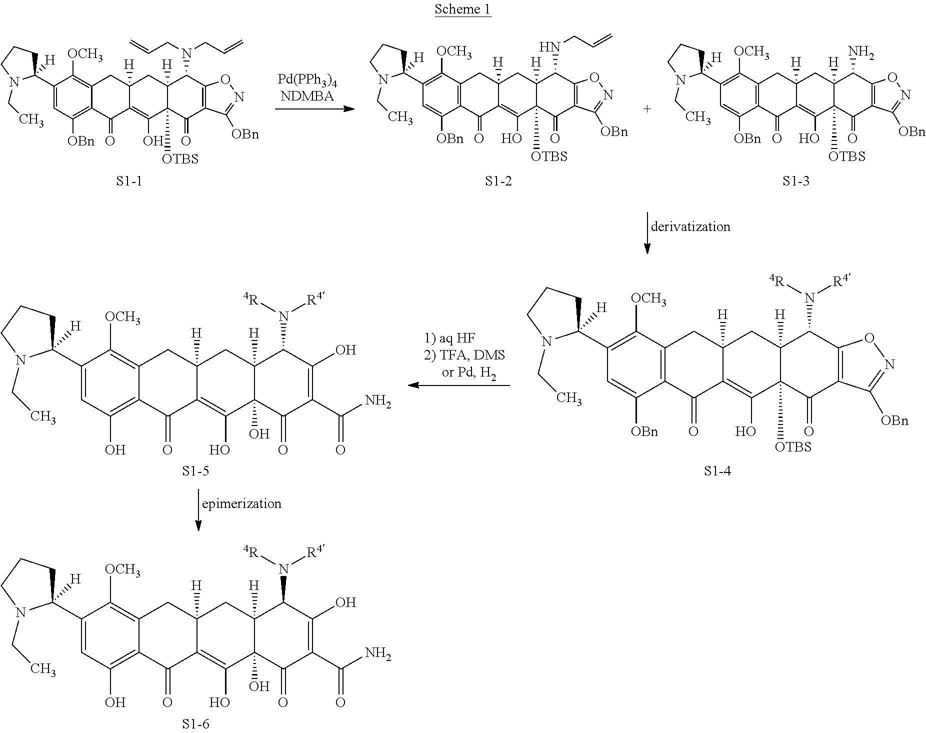

The following compounds were prepared per Scheme 1.

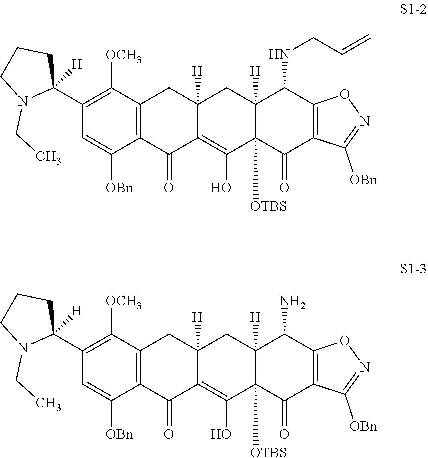 [SMILES: C=CCN[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5CC)c(OC)c4C[C@H]3C[C@@H]12 ; CCN1CCC[C@H]1c1cc(OCc2ccccc2)c2c(c1OC)C[C@H]1C[C@H]3[C@H](N)c4onc(OCc5ccccc5)c4C(=O)[C@@]3(O[Si](C)(C)C(C)(C)C)C(O)=C1C2=O]

General Procedure A (de-allylation): To a mixture of compound S1-1 (498 mg, 0.56 mmol, 1 eq, prepared according to literature procedures including WO 2014036502), 1,3-dimethylbarbituric acid (439 mg, 2.81 mmol, 5 eq) and Pd(PPh₃)₄ (32 mg, 0.028 mmol, 0.05 eq) was added DCM (5 mL) under nitrogen. The resulting reaction solution was stirred at rt for 5 h. The reaction mixture was quenched with aqueous saturated sodium bicarbonate (bubbling). The resulting reaction mixture was stirred at rt for 10 min, and extracted with dichloromethane (3×10 mL). The combined organic extracts were dried over anhydrous sodium sulfate, filtered and concentrated under reduced pressure. The residue was purified by flash chromatography on silica gel using 10%→100% EtOAc/hexanes to yield the desired product S1-2 (82 mg, 17%, MS (ESI) m/z 846.47 (M+H)) and S1-3 (307 mg, 68%).

S1-3: ¹H NMR (400 MHz, CDCl₃) δ 16.54 (s, 1H), 7.42-7.41 (m, 2H), 7.37-7.34 (m, 2H), 7.27-7.15 (m, 7H), 5.29, 5.25 (ABq, J=12.2 Hz, 2H), 5.16, 5.07 (ABq, J=12.2 Hz, 2 H), 3.82 (br s, 1H), 3.61 (t, J=8.5 Hz, 1H), 3.48 (s, 3H), 3.32-3.28 (m, 1H), 2.95 (dd, J=4.3, 15.3 Hz, 1H), 2.69-2.59 (m, 1H), 2.52-2.43 (m, 2H), 2.18-1.98 (m, 5H), 1.88-1.73 (m, 2H), 1.56-1.38 (m, 2H), 0.90 (t, J=7.3 Hz, 3H), 0.63 (s, 9H), 0.11 (s, 3H), 0.00 (s, 3H); MS (ESI) m/z 806.51 (M+H).

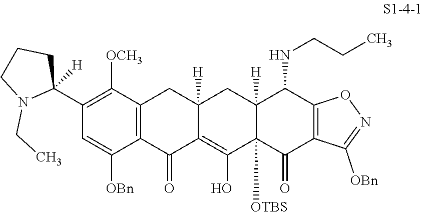 [SMILES: CCCN[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5CC)c(OC)c4C[C@H]3C[C@@H]12]

General procedure B-1 (reductive alkylation): To a solution of amine S1-3 (40 mg, 0.05 mmol, 1.0 eq) in DCM (1 mL) was added HOAc (5.7 μL, 0.1 mmol, 2 eq) and STAB (16 mg, 0.08 mmol, 1.5 eq) at 0° C. Then propionaldehyde (3.6 μL, 0.05 mmol, 1.0 eq) was added. The resulting reaction mixture was stirred at 0° C. for 2 h. Saturated NaHCO₃ was added. The resulting mixture was extracted with DCM (10 mL). The organic phase was dried over Na₂SO₄, filtered and concentrated under reduced pressure. The resulting crude product S1-4-1 was used directly for the next reaction: MS (ESI) m/z 848.48 (M+H).

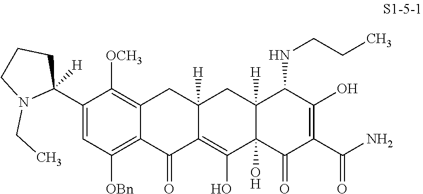 [SMILES: CCCN[C@@H]1C(O)=C(C(N)=O)C(=O)[C@@]2(O)C(O)=C3C(=O)c4c(OCc5ccccc5)cc([C@@H]5CCCN5CC)c(OC)c4C[C@H]3C[C@@H]12]

General Procedure C (HF desilylation): Aqueous HF (48-50%, 0.1 mL) was added to a solution of compound S1-4-1 (0.05 mmol, 1 eq) in CH₃CN (1 mL) in apolypropylene reaction vessel at rt. The mixture was stirred vigorously at rt overnight and poured slowly into saturated aqueous NaHCO₃(3 mL) (vigorously bubbling). The resulting mixture was extracted with EtOAc (10 mL). The organic phase was washed with brine, dried over anhydrous sodium sulfate and concentrated under reduced pressure. The residue was used directly in the next step without further purification (MS (ESI) m/z 734.40 (M+H)).

General Procedure D-1 (global deprotection): To a solution of the above intermediate in TFA (1 mL) was added dimethylsulfide (0.1 mL). The resulting reaction solution was stirred at rt overnight. The reaction was evaporated and the residue was dissolved in 0.05 N HCl in water. The resulting solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 N HCl/water; Solvent B: CH₃CN; injection volume: 2.0 mL (0.05 NHCl/water); gradient: 10-25% B in A over 20 min; mass-directed fraction collection]. Fractions containing the desired product were collected and freeze-dried to yield compound S1-5-1 (14.3 mg, 46% over 3 steps): ¹H NMR (400 MHz, CD₃OD, dihydrochloride salt) δ 7.14 (s, 1H), 4.87-4.84 (m, 1H), 3.90 (s, 1H), 3.87-3.81 (m, 1H), 3.68 (s, 3H), 3.37-3.29 (m, 2H), 3.28-3.07 (m, 4H), 3.01-2.88 (m, 2H), 2.62-2.55 (m, 1H), 2.43-2.24 (m, 5H), 1.83-1.73 (m, 2H), 1.64-1.54 (m, 1H), 1.26 (t, J=7.3 Hz, 3H), 1.03 (t, J=7.3 Hz, 3H); MS (ESI) m/z 556.30 (M+H).

The following compounds were prepared by using general procedures B-1, C, and D-1.

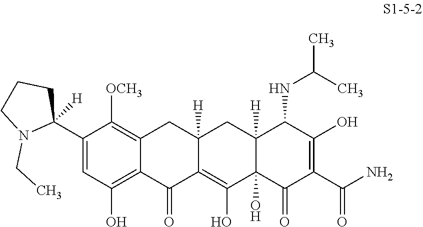 [SMILES: CCN1CCC[C@H]1c1cc(O)c2c(c1OC)C[C@H]1C[C@H]3[C@H](NC(C)C)C(O)=C(C(N)=O)C(=O)[C@@]3(O)C(O)=C1C2=O]

Compound S1-5-2 was prepared from compound S1-3 and acetone: ¹H NMR (400 MHz, CD₃OD, dihydrochloride salt) δ 7.15 (s, 1H), 4.90-4.85 (m, 1H), 3.99 (s, 1H), 3.88-3.82 (m, 2H), 3.68 (s, 3H), 3.38-3.33 (m, 1H), 3.25 (dd, J=16.0, 4.6 Hz, 1H), 3.20-3.08 (m, 2H), 3.02-2.94 (m, 1H), 2.87 (d, J=12.4 Hz, 1H), 2.62-2.55 (m, 1H), 2.42-2.37 (m, 5H), 1.65-1.56 (m, 1H), 1.44 (d, J=6.4 Hz, 3H), 1.40 (d, J=6.4 Hz, 3H), 1.26 (t, J=7.3 Hz, 3 H); MS (ESI) m/z 556.31 (M+H).

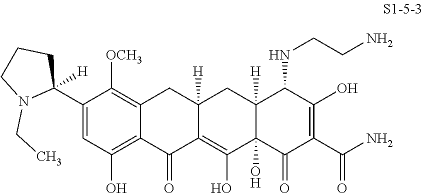 [SMILES: CCN1CCC[C@H]1c1cc(O)c2c(c1OC)C[C@H]1C[C@H]3[C@H](NCCN)C(O)=C(C(N)=O)C(=O)[C@@]3(O)C(O)=C1C2=O]

Compound S1-5-3 was prepared from compound S1-3 and BocNHCH₂CHO: ¹H NMR (400 MHz, CD₃OD, trihydrochloride salt) δ 7.11 (s, 1H), 4.09 (s, 1H), 3.78-3.87 (m, 3H), 3.68 (s, 3H), 3.60-3.65 (m, 1H), 3.39-3.43 (m, 2H), 2.93-3.24 (m, 5H), 2.55-2.62 (m, 1H), 2.23-2.40 (m, 6H), 1.52-1.62 (m, 1H), 1.25 (t, J=7.2 Hz, 3H); MS (ESI) m/z 557.3 (M+H).

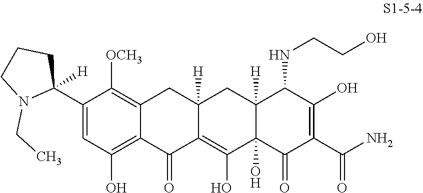 [SMILES: CCN1CCC[C@H]1c1cc(O)c2c(c1OC)C[C@H]1C[C@H]3[C@H](NCCO)C(O)=C(C(N)=O)C(=O)[C@@]3(O)C(O)=C1C2=O]

Compound S1-5-4 was prepared from compound S1-3 and TBSOCH₂CHO: ¹H NMR (400 MHz, CD₃OD, dihydrochloride salt) δ 7.12 (s, 1H), 4.01 (s, 1H), 3.80-3.91 (m, 4H), 3.67 (s, 3H), 3.39-3.50 (m, 2H), 3.05-3.24 (m, 4H), 2.88-3.00 (m, 2H), 2.55-2.61 (m, 1H), 2.20-2.40 (m, 5H), 1.55-1.62 (m, 1H), 1.25 (t, J=8.0 Hz, 3H); MS (ESI) m/z 558.3 (M+H).

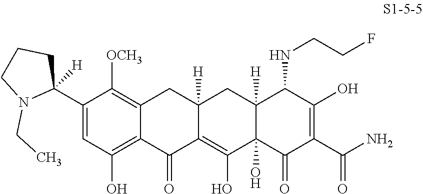 [SMILES: CCN1CCC[C@H]1c1cc(O)c2c(c1OC)C[C@H]1C[C@H]3[C@H](NCCF)C(O)=C(C(N)=O)C(=O)[C@@]3(O)C(O)=C1C2=O]

Compound S1-5-5 was prepared from compound S1-3 and FCH₂CHO (prepared from the corresponding alcohol according to the literature procedure in WO 2011146089 A1): ¹H NMR (400 MHz, CD₃OD, dihydrochloride salt) δ 7.13 (s, 1H), 4.03 (s, 1H), 3.69-3.88 (m, 4 H), 3.66 (s, 3H), 3.25-3.38 (m, 3H), 3.05-3.23 (m, 2H), 2.89-3.00 (m, 2H), 2.55-2.61 (m, 1 H), 2.21-2.42 (m, 6H), 1.56-1.66 (m, 1H), 1.23 (t, J=7.2 Hz, 3H); MS (ESI) m/z 560.3 (M+H).

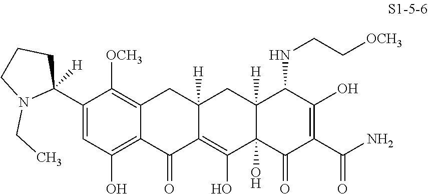 [SMILES: CCN1CCC[C@H]1c1cc(O)c2c(c1OC)C[C@H]1C[C@H]3[C@H](NCCOC)C(O)=C(C(N)=O)C(=O)[C@@]3(O)C(O)=C1C2=O]

Compound S1-5-6 was prepared from compound S1-3 and CH₃OCH₂CHO (prepared from the corresponding alcohol according to the literature procedure in WO 2011146089 A1): ¹H NMR (400 MHz, CD₃OD, dihydrochloride salt) δ 7.03 (s, 1H), 3.88 (s, 1H), 3.69-3.75 (m, 1H), 3.61-3.64 (m, 2H), 3.67 (s, 3H), 3.38-3.42 (m, 2H), 3.30 (s, 3H), 3.18-3.25 (m, 3 H), 2.95-3.15 (m, 2H), 2.75-2.90 (m, 2H), 2.45-2.51 (m, 1H), 2.09-2.31 (m, 5H), 1.44-1.54 (m, 1H), 1.12 (t, J=7.2 Hz, 3H); MS (ESI) m/z 572.3 (M+H).

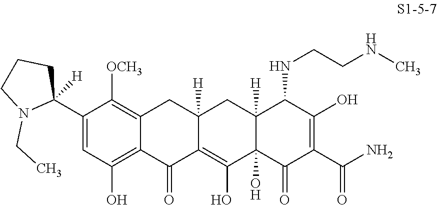 [SMILES: CCN1CCC[C@H]1c1cc(O)c2c(c1OC)C[C@H]1C[C@H]3[C@H](NCCNC)C(O)=C(C(N)=O)C(=O)[C@@]3(O)C(O)=C1C2=O]

Compound S1-5-7 was prepared from compound S1-3 and BocN(CH₃)CH₂CHO: ¹H NMR (400 MHz, CD₃OD, trihydrochloride salt) δ 7.11 (s, 1H), 4.09 (s, 1H), 3.79-3.89 (m, 2 H), 3.67 (s, 3H), 3.55-3.60 (m, 2H), 3.30 (s, 3H), 2.95-3.18 (m, 4H), 2.79 (s, 3H), 2.55-2.61 (m, 1H), 2.21-2.31 (m, 6H), 1.56-1.63 (m, 1H), 1.25 (t, J=7.2 Hz, 3H); MS (ESI) m/z 571.3 (M+H).

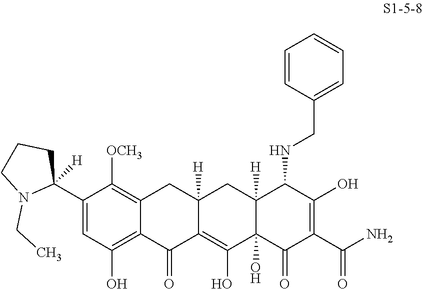 [SMILES: CCN1CCC[C@H]1c1cc(O)c2c(c1OC)C[C@H]1C[C@H]3[C@H](NCc4ccccc4)C(O)=C(C(N)=O)C(=O)[C@@]3(O)C(O)=C1C2=O]

Compound S1-5-8 was prepared from compound S1-3 and PhCHO: ¹H NMR (400 MHz, CD₃OD, dihydrochloride salt) δ 7.55-7.62 (m, 2H), 7.45-7.51 (m, 3H), 7.09 (s, 1H), 4.47-4.52 (m, 2H), 3.80-3.75 (m, 2H), 3.67 (s, 3H), 3.09-3.23 (m, 4H), 2.83-2.93 (m, 2H), 2.55-2.61 (m, 1H), 2.21-2.40 (m, 5H), 2.00-2.08 (m, 1H), 1.51-1.63 (m, 1H), 1.25 (t, J=7.2 Hz, 3H); MS (ESI) m/z 604.3 (M+H).

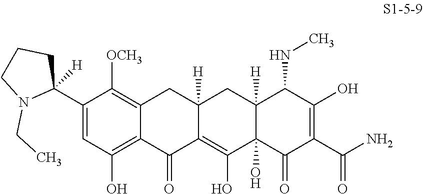 [SMILES: CCN1CCC[C@H]1c1cc(O)c2c(c1OC)C[C@H]1C[C@H]3[C@H](NC)C(O)=C(C(N)=O)C(=O)[C@@]3(O)C(O)=C1C2=O]

Compound S1-5-9 was prepared from compound S1-2 (44 mg, 0.052 mmol, 1 eq) and HCHO by using general procedure B-1 and C, followed by the following general procedure D-2.

General procedure D-2: Pd—C (10 wt %, 5 mg) was added in one portion into a solution of the above crude product in a mixture of CH₃OH (1 mL) and HCl/water (1 N, 130 μL, 0.13 mmol, 2.5 eq) at rt. The reaction vessel was sealed and purged with hydrogen by briefly evacuating the flask followed by flushing with hydrogen gas (1 atm). The reaction mixture was stirred under a hydrogen atmosphere (1 atm) at rt for 1 h 30 min. More Pd—C (10 wt %, 5 mg) was added and the resulting reaction mixture was stirred under a hydrogen atmosphere (1 atm) at rt for 1 h. The reaction was filtered through a small Celite pad. The cake was washed with CH₃OH. The filtrate was concentrated. The residue was purified by preparative reverse phase HPLC on a Waters Autopurification system using a Phenomenex Polymerx 10μ RP-γ 100A column [10 pm, 150×21.20 mm; flow rate, 20 mL/min; Solvent A: 0.05 NHCl/water; Solvent B: CH₃CN; injection volume: 3.0 mL (0.05 N HCl/water); gradient: 5→25% B in A over 15 min; mass-directed fraction collection]. Fractions containing the desired product were collected and freeze-dried to yield compound S1-5-9 (12.3 mg): ¹H NMR (400 MHz, CD₃OD, dihydrochloride salt) δ 7.15 (s, 1H), 4.96-4.89 (m, 1H), 3.84-3.81 (m, 2H), 3.68 (s, 3H), 3.36-3.33 (m, 1H), 3.27-2.99 (m, 5H), 2.93 (s, 3H), 2.88-2.83 (m, 1H), 2.62-2.55 (m, 1H), 2.42-2.24 (m, 4H), 1.63-1.54 (m, 1H), 1.26 (t, J=7.3 Hz, 3H); MS (ESI) m/z 528.23 (M+H).

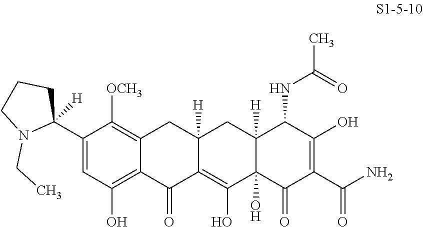 [SMILES: CCN1CCC[C@H]1c1cc(O)c2c(c1OC)C[C@H]1C[C@H]3[C@H](NC(C)=O)C(O)=C(C(N)=O)C(=O)[C@@]3(O)C(O)=C1C2=O]

General procedure B-2 (acylation/sulfonylation): To a solution of compound S1-3 (43 mg, 0.053 mmol, 1 eq) and TEA (30 μL, 0.21 mmol, 4 eq) in DCM (3 mL) was added acetic anhydride (16 μL, 0.16 mmol, 3 eq) at 0° C. The resulting reaction mixture was stirred at 0° C. and allowed to warm up to rt overnight. The reaction was diluted with DCM, washed with saturated sodium bicarbonate and brine. The resulting organic phase was dried over anhydrous sodium sulfate, filtered, and concentrated. The crude product was subjected to the general procedure C for HF desilylation at 50° C. and general procedure D-1 to give S1-5-10 (11.2 mg, 36% over 3 steps):

¹H NMR (400 MHz, CD₃OD, hydrochloride salt) δ 7.04 (s, 1H), 3.79-3.85 (m, 2H), 3.69 (s, 3H), 3.05-3.21 (m, 4H), 2.90-3.00 (m, 1H), 2.53-2.70 (m, 2H), 2.21-2.45 (m, 6H), 2.05 (s, 3H), 1.51-1.60 (m, 1H), 1.25 (t, J=7.2 Hz, 3H); MS (ESI) m/z 556.3 (M+H).

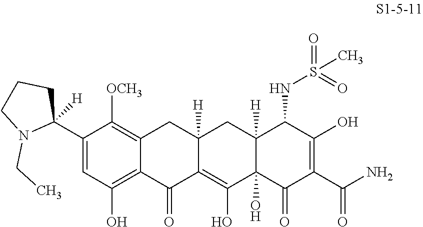 [SMILES: CCN1CCC[C@H]1c1cc(O)c2c(c1OC)C[C@H]1C[C@H]3[C@H](NS(C)(=O)=O)C(O)=C(C(N)=O)C(=O)[C@@]3(O)C(O)=C1C2=O]

Compound S1-5-11 was prepared from compound S1-3 and Ms₂O following the same procedure as for compound S1-5-10: ¹H NMR (400 MHz, CD₃OD, hydrochloride salt) δ 7.01 (s, 1H), 4.15 (m, 1H), 3.75-3.83 (m, 2H), 3.69 (s, 3H), 3.16-3.40 (m, 4H), 2.92-3.11 (m, 3 H), 2.41-2.61 (m, 3H), 2.22-2.38 (m, 5H), 1.75-1.83 (m, 1H), 1.25 (t, J=7.2 Hz, 3H); MS (ESI) m/z 592.3 (M+H).

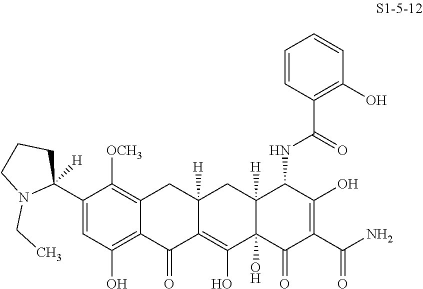 [SMILES: CCN1CCC[C@H]1c1cc(O)c2c(c1OC)C[C@H]1C[C@H]3[C@H](NC(=O)c4ccccc4O)C(O)=C(C(N)=O)C(=O)[C@@]3(O)C(O)=C1C2=O]

To a mixture of amine S1-3 (48 mg, 0.06 mmol, 1.0 eq), HOBt (12 mg, 0.09 mmol, 1.5 eq) and EDC (17 mg, 0.09 mmol, 1.5 eq) in 10 mL RBF was added DCM (1 mL) under nitrogen. Then EtNⁱPr₂ (21 μL, 0.12 mmol, 2 eq) and salicyclic acid (9 mg, 0.07 mmol, 1.1 eq) were added subsequently. The resulting reaction mixture was stirred at rt for 5 days. The resulting dark reaction mixture was extracted with DCM (10 mL). The organic phase was washed with brine, dried over Na₂SO₄, filtered and concentrated under reduced pressure. The residue was purified by flash column chromatography (10 g silica gel column, 10-80% EtOAc/Hexane) to give the desired product (13 mg, 23%): MS (ESI) m/z 926.53 (M+H).

The above product was subjected to general procedure C and D-1 to give compound S1-5-12: ¹H NMR (400 MHz, CD₃OD, hydrochloride salt) δ 7.83 (d, J=7.3 Hz, 1H), 7.40 (t, J=7.3 Hz, 1H), 7.03 (s, 1H), 6.94-6.90 (m, 2H), 5.07-5.06 (m, 1H), 3.81-3.76 (m, 2H), 3.65 (s, 3H), 3.21-3.06 (m, 4H), 2.98-2.94 (m, 1H), 2.62-2.58 (m, 2H), 2.45-2.22 (m, 5H), 1.74-1.67 (m, 1H), 1.23 (t, J=7.3 Hz, 3H); MS (ESI) m/z 634.39 (M+H).

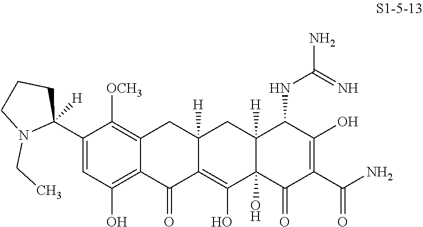 [SMILES: CCN1CCC[C@H]1c1cc(O)c2c(c1OC)C[C@H]1C[C@H]3[C@H](NC(=N)N)C(O)=C(C(N)=O)C(=O)[C@@]3(O)C(O)=C1C2=O]

To a solution of amine S1-3 (82 mg, 0.10 mmol, 1.0 eq) was subjected to general procedure C to give desilylated product 74 mg. To a solution of this intermediate (42 mg, 0.06 mmol, 1.0 eq) in DCM (1 mL) was added HgCl₂ (33 mg, 0.12 mmol, 2.2 eq) and TEA (30 μL, 0.21 mmol, 3.5 eq) at 0° C. Then 1,3-bis(tert-butoxycarbonyl)-2-methylisothiourea (39 mg, 0.12 mmol, 2.2 eq) was added. The resulting reaction mixture was allowed to warm up to rt and stirred overnight. The resulting reaction mixture was filtered, washed with DCM (10 mL). The filtrate was concentrated under reduced pressure. The residue was purified by flash column chromatography (10 g silica gel column, 10% CH₃OH/DCM) to give the desired product (20 mg, 35%): MS (ESI) m/z 934.57 (M+H).

The above product was subjected to general procedure D-1 to give compound S1-5-13: ¹H NMR (400 MHz, CD₃OD, dihydrochloride salt) δ 7.06 (s, 1H), 4.30 (s, 1H), 3.78-3.84 (m, 1H), 3.68 (s, 3H), 3.32-3.40 (m, 2H), 3.08-3.17 (m, 3H), 2.90-3.00 (m, 1H), 2.53-2.60 (m, 3H), 2.21-2.39 (m, 5H), 1.58-1.64 (m, 1H), 1.22 (t, J=6.8 Hz, 3H); MS (ESI) m/z 556.3 (M+H).

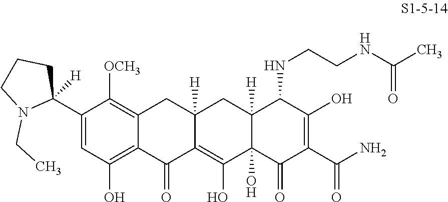 [SMILES: CCN1CCC[C@H]1c1cc(O)c2c(c1OC)C[C@H]1C[C@H]3[C@H](NCCNC(C)=O)C(O)=C(C(N)=O)C(=O)[C@@]3(O)C(O)=C1C2=O]

Compound S1-5-14 was prepared from compound S1-3 and BocNHCH₂CHO by using general procedure B-1. The resulting product was then treated with 4 NHCl in dioxane (1 mL) for 30 min and concentrated. The residue was subjected to general procedure B-2, C and D-1 to give desired product S1-5-14: ¹H NMR (400 MHz, CD₃OD, dihydrochloride salt) δ 7.10 (s, 1 H), 3.99 (s, 1H), 3.79-3.83 (m, 1H), 3.67 (s, 3H), 3.55-3.60 (m, 1H), 3.45-3.51 (m, 3H), 3.31-3.35 (m, 1H), 3.05-3.27 (m, 4H), 2.92-3.00 (m, 1H), 2.79-2.83 (m, 1H), 2.55-2.60 (m, 1H), 2.20-2.40 (m, 5H), 1.98 (s, 3H), 1.52-1.62 (m, 1H), 1.22 (t, J=7.2 Hz, 3H); MS (ESI) m/z 599.3 (M+H).

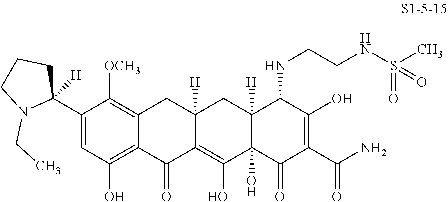 [SMILES: CCN1CCC[C@H]1c1cc(O)c2c(c1OC)C[C@H]1C[C@H]3[C@H](NCCNS(C)(=O)=O)C(O)=C(C(N)=O)C(=O)[C@@]3(O)C(O)=C1C2=O]

Compound S1-5-15 was prepared similarly to compound S1-5-14: ¹H NMR (400 MHz, CD₃OD, dihydrochloride salt) δ 7.08 (s, 1H), 4.05 (s, 1H), 3.78-3.85 (m, 2H), 3.68 (m, 5H), 3.45-3.52 (m, 6H), 3.09-3.20 (m, 2H), 2.89-3.00 (m, 2H), 2.55-2.62 (m, 1H), 2.21-2.51 (m, 6H), 1.53-1.63 (m, 1H), 1.23 (t, J=7.2 Hz, 3H); MS (ESI) m/z 635.3 (M+H).

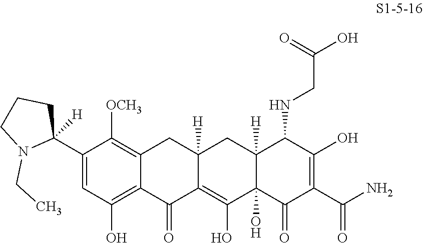 [SMILES: CCN1CCC[C@H]1c1cc(O)c2c(c1OC)C[C@H]1C[C@H]3[C@H](NCC(=O)O)C(O)=C(C(N)=O)C(=O)[C@@]3(O)C(O)=C1C2=O]

General procedure B-3 (substitution): To a solution of amine S1-3 (42 mg, 0.05 mmol, 1.0 eq) in DMF (0.7 mL) was added BrCH₂CO₂Bu (8 μL, 0.05 mmol, 1 eq) and ⁱPr₂NEt (45 μL, 0.25 mmol, 5 eq). The resulting reaction mixture was stirred at rt overnight and heated to 50° C. for 6 h. The resulting reaction mixture was diluted with EtOAc, washed with brine, dried over Na₂SO₄, filtered and concentrated under reduced pressure. The residue was used directly for the next reaction.

The crude product was then subjected to general procedure C and D-1 to give desired product S1-5-16: ¹H NMR (400 MHz, CD₃OD, dihydrochloride salt) δ 7.08 (s, 1H), 4.19 (s, 2 H), 3.99 (s, 1H), 3.78-3.83 (m, 1H), 3.68 (s, 3H), 3.05-3.22 (m, 3H), 2.83-3.00 (m, 2H), 2.52-2.61 (m, 1H), 2.19-2.40 (m, 5H), 1.56-1.67 (m, 1H), 1.22 (t, J=7.2 Hz, 3H); MS (ESI) m/z 572.2 (M+H).

The following compounds were prepared by using general procedures B-3, C, and D-1.

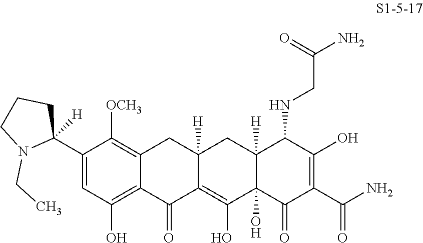 [SMILES: CCN1CCC[C@H]1c1cc(O)c2c(c1OC)C[C@H]1C[C@H]3[C@H](NCC(N)=O)C(O)=C(C(N)=O)C(=O)[C@@]3(O)C(O)=C1C2=O]

Compound S1-5-17 was prepared from compound S1-3 and BrCH₂CONH₂: ¹H NMR (400 MHz, CD₃OD, dihydrochloride salt) δ 7.11 (s, 1H), 4.15 (s, 2H), 3.98 (s, 1H), 3.79-3.84 (m, 1H), 3.68 (s, 3H), 3.09-3.24 (m, 3H), 2.83-3.00 (m, 2H), 2.55-2.63 (m, 1H), 2.20-2.40 (m, 5H), 1.55-1.65 (m, 1H), 1.22 (t, J=7.2 Hz, 3H); MS (ESI) m/z 571.3 (M+H).

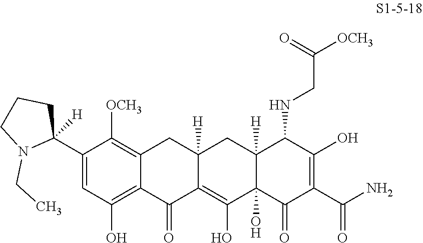 [SMILES: CCN1CCC[C@H]1c1cc(O)c2c(c1OC)C[C@H]1C[C@H]3[C@H](NCC(=O)OC)C(O)=C(C(N)=O)C(=O)[C@@]3(O)C(O)=C1C2=O]

Compound S1-5-18 was prepared from compound S1-3 and BrCH₂CO₂Me: ¹H NMR (400 MHz, CD₃OD, dihydrochloride salt) δ 7.11 (s, 1H), 4.26 (s, 2H), 4.03 (s, 1H), 3.84 (s, 3H), 3.79-3.82 (m, 1H), 3.68 (s, 3H), 3.09-3.24 (m, 3H), 2.87-3.00 (m, 2H), 2.55-2.62 (m, 1H), 2.20-2.50 (m, 5H), 1.55-1.63 (m, 1H), 1.21 (t, J=7.2 Hz, 3H); MS (ESI) m/z 586.3 (M+H).

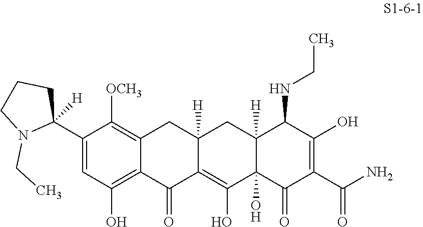 [SMILES: CCN[C@H]1C(O)=C(C(N)=O)C(=O)[C@@]2(O)C(O)=C3C(=O)c4c(O)cc([C@@H]5CCCN5CC)c(OC)c4C[C@H]3C[C@@H]12]

To a solution of the corresponding C-4 epimer (71 mg, 0.12 mmol, 1 eq, prepared per literature procedures including WO 2014036502) in CH₃OH (1 mL) was added pyridine (38 μL, 0.47 mmol, 4 eq) at rt. The resulting reaction solution was stirred at rt for 3 days. The reaction was concentrated to give a yellow solid, which was dissolved in 0.05 N HCl in water. The resulting reaction solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 N HCl/water; Solvent B: CH₃CN; injection volume: 2.0 mL (0.05 NHCl/water); gradient: 10→25% B in A over 20 min; mass-directed fraction collection]. Fractions containing the desired product were collected and freeze-dried to yield compound S1-6-1 (27.2 mg, 38%): ¹H NMR (400 MHz, CD₃OD, dihydrochloride salt) δ 7.18 (s, 1H), 4.88-4.84 (m, 1H), 4.72 (d, J=4.0 Hz, 1H), 3.88-3.82 (m, 1H), 3.67 (s, 3H), 3.41-3.30 (m, 3H), 3.27-3.22 (m, 1H), 3.20-3.06 (m, 2H), 3.03-2.98 (m, 1H), 2.96-2.88 (m, 2H), 2.61-2.54 (m, 1H), 2.41 (t, J=14.8 Hz, 1H), 2.36-2.23 (m, 3 H), 2.18-2.14 (m, 1H), 1.56-1.46 (m, 1H), 1.44 (t, J=7.2 Hz, 3H), 1.26 (t, J=7.6 Hz, 3H); MS (ESI) m/z 541.4 (M+H).

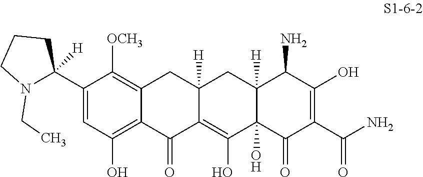 [SMILES: CCN1CCC[C@H]1c1cc(O)c2c(c1OC)C[C@H]1C[C@H]3[C@@H](N)C(O)=C(C(N)=O)C(=O)[C@@]3(O)C(O)=C1C2=O]

To a suspension of 7-methoxy-8-[(2S)-1-ethyl-2-pyrrolidinyl]-6-demethyl-6-deoxytetracycline (550 mg, 0.89 mmol, 1 eq, prepared per literature procedures including Org. Process Res. Dev., 2016, 20 (2), 284-296.) in DMF (4.4 mL) was added a solution of NH₂OH (109 μL, 1.78 mmol, 2 eq) in water (109 μL) at rt. The resulting reaction mixture was stirred at 80° C. overnight with a needle in the septum to open to air. The resulting dark brown reaction solution was cooled to rt, and dropped into stirring MTBE (220 mL) to give a suspension.

The solid was collected by filtration and washed with MTBE. The solid was then dried under vacuum. The solid was then dissolved in TFA (4 mL). Pd/C (10 wt %, 80 mg) was added. The reaction vessel was sealed and purged with hydrogen by briefly evacuating the flask followed by flushing with hydrogen gas (1 atm). The reaction mixture was stirred under a hydrogen atmosphere (1 atm) at rt overnight. More Pd—C (10 wt %, 80 mg) was added and the resulting reaction mixture was stirred under a hydrogen atmosphere (1 atm) at rt overnight. The reaction was concentrated and diluted with CH₃OH. The mixture was filtered through a small Celite pad. The cake was washed with. The filtrate was concentrated. The residue was purified by preparative reverse phase HPLC CH₃OH on a Waters Autopurification system using a Phenomenex Polymerx 10μ RP-γ 100A column [10 μm, 150×21.20 mm; flow rate, 20 mL/min; Solvent A: 0.05 NHCl/water; Solvent B: CH₃CN; injection volume: 3.0 mL (0.05 N HCl/water); gradient: 10→20% B in A over 15 min; mass-directed fraction collection. Fractions containing the desired product were collected and freeze-dried to yield compound S1-6-2 (91 mg): ¹H NMR (400 MHz, CD₃OD, dihydrochloride salt) δ 7.12 (s, 1H), 4.93-4.85 (m, 1H), 4.76 (d, J=4.8 Hz, 1H), 3.86-3.81 (m, 1H), 3.67 (s, 3H), 3.37-3.31 (m, 1H), 3.25 (dd, J=15.2, 4.0 Hz, 1H), 3.20-3.07 (m, 2H), 2.90-2.82 (m, 2H), 2.62-2.56 (m, 1H), 2.43 (t, J=14.8 Hz, 1H), 2.36-2.23 (m, 3H), 2.16-2.12 (m, 1H), 1.53-1.43 (m, 1H), 1.25 (t, J=7.2 Hz, 3H); MS (ESI) m/z 514.36 (M+H).

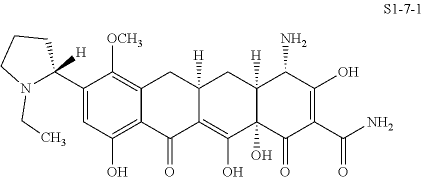 [SMILES: CCN1CCC[C@@H]1c1cc(O)c2c(c1OC)C[C@H]1C[C@H]3[C@H](N)C(O)=C(C(N)=O)C(=O)[C@@]3(O)C(O)=C1C2=O]

Compound S1-7-1 was prepared from the enantiomer of the left-hand side (LHS) and diallylenone S2-3 according to literature procedures including WO 2014036502: ¹H NMR (400 MHz, CD₃OD, dihydrochloride salt) δ 7.11 (s, 1H), 4.77 (dd, J=10.8, 8.0 Hz, 1H), 3.92-3.86 (m, 2H), 3.75 (s, 3H), 3.37-3.29 (m, 1H), 3.25-3.10 (m, 3H), 3.01-2.93 (m, 1H), 2.68 (dt, J=12.4, 1.2 Hz, 1H), 2.63-2.54 (m, 1H), 2.38 (t, J=14.8 Hz, 1H), 2.32-2.24 (m, 3H), 2.17-2.07 (m, 1H), 1.64-1.55 (m, 1H), 1.28 (t, J=7.6 Hz, 3H); MS (ESI) m/z 514.36 (M+H).

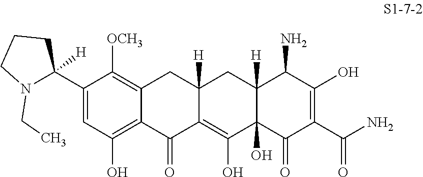 [SMILES: CCN1CCC[C@H]1c1cc(O)c2c(c1OC)C[C@@H]1C[C@@H]3[C@@H](N)C(O)=C(C(N)=O)C(=O)[C@]3(O)C(O)=C1C2=O]

Compound S1-7-2 was prepared from the normal LHS and the enantiomer of the diallylenone according to literature procedures including WO 2014036502: ¹H NMR (400 MHz, CD₃OD, dihydrochloride salt) δ 7.06 (s, 1H), 4.76 (dd, J=10.4, 7.6 Hz, 1H), 3.91-3.85 (m, 2H), 3.75 (s, 3H), 3.37-3.30 (m, 1H), 3.25-3.09 (m, 3H), 3.00-2.92 (m, 1H), 2.67-2.57 (m, 2H), 2.39 (t, J=14.8 Hz, 1H), 2.34-2.24 (m, 3H), 2.17-2.09 (m, 1H), 1.65-1.56 (m, 1 H), 1.27 (t, J=7.2 Hz, 3H); MS (ESI) m/z 514.36 (M+H).

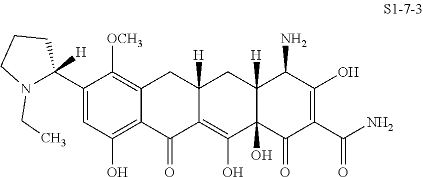 [SMILES: CCN1CCC[C@@H]1c1cc(O)c2c(c1OC)C[C@@H]1C[C@@H]3[C@@H](N)C(O)=C(C(N)=O)C(=O)[C@]3(O)C(O)=C1C2=O]

Compound S1-7-3 was prepared from the enantiomer of LHS and the enantiomer of the diallylenone according to literature procedures including WO 2014036502: ¹H NMR (400 MHz, CD₃OD, dihydrochloride salt) δ 7.15 (s, 1H), 4.94-4.85 (m, 1H), 3.91 (s, 1H), 3.80-3.72 (m, 1H), 3.68 (s, 3H), 3.37-3.07 (m, 4H), 3.00-2.91 (m, 1H), 2.70-2.67 (m, 1H), 2.62-2.56 (m, 1H), 2.45-2.23 (m, 5H), 1.65-1.56 (m, 1H), 1.26 (t, J=7.2 Hz, 3H); MS (ESI) m/z 514.36 (M+H).

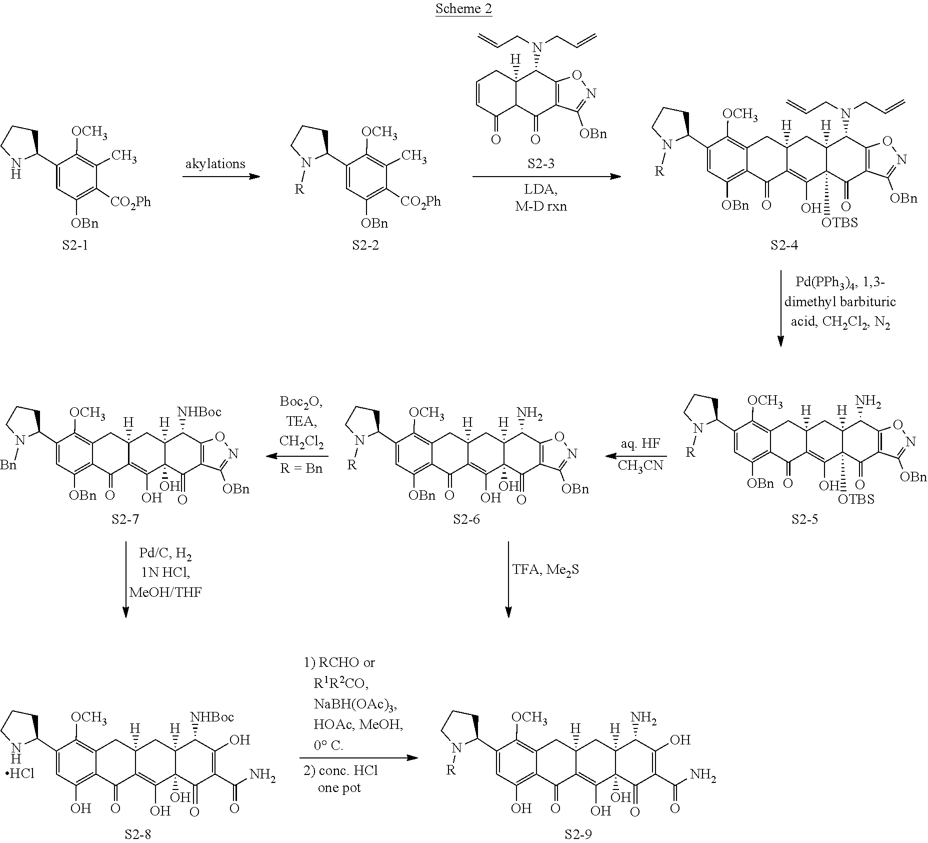

The following compounds were prepared per Scheme 2.

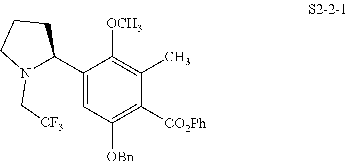

Compound S2-1 (125 mg, 0.299 mmol, 1 eq, prepared per literature procedures: Org. Process Res. Dev., 2016, 20 (2), 284-296) and NaBH₃CN (76 mg, 1.209 mmol, 4 eq) were added to a mixture of CH₂Cl₂ and CH₃CN (0.8+0.8 mL). The flask cooled down to 0° C., followed by the addition of trifluoroacetic acid (0.092 mL, 1.202 mmol, 4 eq) and trifluoroacetaldehyde monohydrate (75% in H₂O, 0.240 mL, 1.50 mmol, 5 eq). The cold bath was removed and the resulting mixture was stirred at room temperature for 2 h. EtOAc was added and the mixture washed with saturated NaHCO₃ solution. The organic phase was concentrated by rotovap. The residue was purified through flash column chromatography to afford the desired product S2-2-1 as a colorless oil (59 mg, 40%, the unreacted SM can also be recovered): ¹H NMR (400 MHz, CDCl₃) δ 7.08-7.50 (m, 11H), 5.09-5.17 (m, 2H), 3.92-4.00 (m, 1H), 3.70 (s, 3H), 3.51-3.60 (m, 1H), 3.08-3.20 (m, 1H), 2.75-2.83 (m, 1H), 2.49-2.57 (m, 1H), 2.40 (s, 3H), 2.20-2.28 (m, 1H), 1.88-2.00 (m, 1H), 1.55-1.65 (m, 1H), 1.21-1.30 (m, 1H); MS (ESI) m/z 500.3 (M+H).

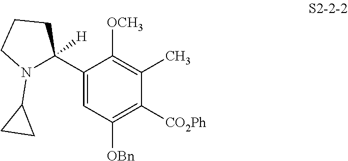

To a flame-dried round-bottom flask, compound S2-1 (125 mg, 0.299 mmol, 1 eq), NaBH₃CN (57 mg, 0.907 mmol, 3 eq) and 4 Å molecular sieves (100 mg) were added, the flask was vacuumed and refilled with N₂. Then anhydrous CH₃OH (2 mL), (1-ethoxycyclopropyl) trimethylsilane (0.240 mL, 1.193 mmol, 4 eq) and HOAc (0.086 mL, 1.500 mmol, 5 eq) were added and the resulted mixture was stirred at 55° C. for 16 h. EtOAc was added and the mixture was filtered through Celite. The filtrate was washed with saturated NaHCO₃ solution. The organic phase was concentrated by rotovap. The residue was purified through flash column chromatography to afford the desired product S2-2-2 as a colorless oil (89 mg, 65%): ¹H NMR (400 MHz, CDCl₃) δ 7.10-7.58 (m, 10H), 7.02 (s, 1H), 5.11 (s, 2H), 3.95-4.01 (m, 1H), 3.71 (s, 3H), 3.21-3.30 (m, 1H), 2.55-2.63 (m, 1H), 2.40 (s, 3H), 2.20-2.30 (m, 1H), 1.78-1.90 (m, 2H), 1.55-1.70 (m, 2H), 0.27-0.35 (m, 2H), 0.00-0.16 (m, 2H); MS (ESI) m/z 458.3 (M+H).

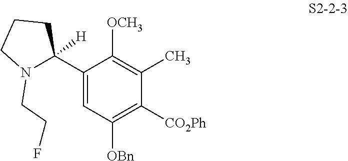

Compound S2-1 (125 mg, 0.299 mmol, 1 eq), N,N-diisopropylethylamine (DIPEA, 0.105 mL, 0.602 mmol, 2 eq) and NaI (5 mg, 0.033 mmol, 0.1 eq) were added into DMF (1 mL), then 2-fluoroethyl bromide (0.045 mL, 0.604 mmol, 2 eq) was added and the resulted mixture was stirred at room temperature for 21 h. EtOAc was added and washed with brine solution. The organic phase was concentrated by rotovap. The residue was purified through flash column chromatography to afford the desired product S2-2-3 as a colorless oil (79 mg, 57%): ¹H NMR (400 MHz, CDCl₃) δ 7.09-7.50 (m, 11H), 5.10-5.15 (m, 2H), 4.30-4.51 (m, 2 H), 3.70 (s, 3H), 3.40-3.50 (m, 1H), 2.79-2.90 (m, 1H), 2.37 (s, 3H), 2.30-2.35 (m, 1H), 2.18-2.26 (m, 1H), 1.82-2.00 (m, 2H), 1.53-1.61 (m, 1H), 1.21-1.30 (m, 1H), 0.82-0.91 (m, 1H); MS (ESI) m/z 464.3 (M+H).

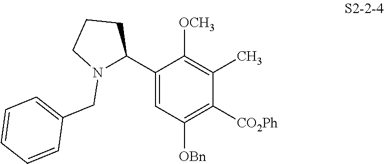

To the pyrrolidine S2-1 (8.74 mmol, 1 eq, crude material), NaI (10 mg), dimethylformamide (DMF, 10 mL) and triethylamine (TEA, 2.82 mL, 20.231 mmol) were added and cooled to 0° C. A solution of benzyl bromide (1.650 mL, 13.867 mmol) in DMF (5 mL) was added. The reaction mixture was stirred at room temperature for 3 h. CH₂Cl₂ (100 mL) was added, and the resulting mixture was washed with brine solution. The organic phase was concentrated under reduced pressure. The residue was purified through flash column chromatography to afford the desired product S2-2-4 as a white solid (3.83 g, 86% over 3 steps): ¹H NMR (400 MHz, CDCl₃) δ 7.09-7.59 (m, 16H), 5.12-5.20 (m, 2H), 3.80-3.90 (m, 2H), 3.74 (s, 3H), 3.03-3.12 (m, 1H), 2.41 (s, 3H), 2.20-2.30 (m, 2H), 1.80-1.94 (m, 2H), 1.60-1.70 (m, 2H); MS (ESI) m/z 508.3 (M+H).

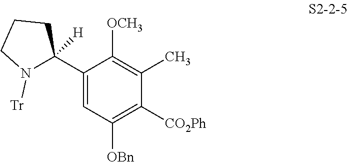

TrCl (87 mg, 0.31 mmol, 1.0 eq) and TEA (48 μL, 0.34 mmol, 1.1 eq) were added to S2-1 (130 mg, 0.31 mmol, 1 eq) in CH₂Cl₂ (3 mL) at rt. The reaction mixture was stirred at rt for 3 days and diluted with DCM. The resulting solution washed with saturated NaHCO₃ and brine, dried over MgSO₄, and concentrated under reduced pressure to give the desired product S2-2-5 as a yellow solid. This crude product was used in subsequent reaction without further purification.

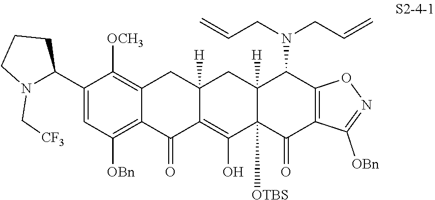 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5CC(F)(F)F)c(OC)c4C[C@H]3C[C@@H]12]

General Procedure E (Michael-Dieckmann annulation): n-BuLi (70 μL, 2.5 M in hexanes, 0.17 mmol, 1.4 eq) was added dropwise to a solution of diisopropylamine (23 μL, 0.17 mmol, 1.4 eq) and TEA HCl (1 mg, 0.005 eq) in THF (1 mL) at −50° C. The reaction mixture was warmed up to −20° C. and re-cooled to below −70° C. A solution of S2-2-1 (59 mg, 0.12 mmol, 1 eq) in THF (1 mL) was added dropwise via a cannula at below −73° C. over 10 min. The resulting red orange solution was stirred at −78° C. for 1 h, and cooled to −100° C. using a EtOH/liquid N₂ bath. A solution of enone S2-3 (64 mg, 0.12 mmol, 1 eq, prepared according to literature procedures including WO 2014036502) in THF (1 mL) was added to the reaction mixture, followed by LHMDS (120 μL, 1.0 M in THF, 0.12 mmol, 1 eq). The reaction mixture was gradually warmed up to −15° C. and stirred at that temperature for 45 min. A saturated aqueous NH₄Cl (20 mL) solution was added to the reaction. The reaction mixture was extracted with EtOAc (40 mL). The organic phase was washed with brine (20 mL), dried over Na₂SO₄, and concentrated under reduced pressure. Flash chromatography on silica gel using 0%→50% EtOAc/hexanes yielded the desired product S2-4-1 as a yellow solid (59 mg, 53%): ¹H NMR (400 MHz, CDCl₃) δ 16.2 (s, 1H), 7.28-7.51 (m, 8H), 6.83-6.95 (m, 3H), 5.79-5.90 (m, 2H), 5.10-5.27 (m, 7H), 3.99-4.13 (m, 2H), 3.68 (s, 3H), 3.03-3.67 (m, 7H), 2.57-2.80 (m, 6H), 1.19-1.26 (m, 6H), 0.85 (s, 9H), 0.27 (s, 3H), 0.15 (s, 3H); MS (ESI) m/z 940.3 (M+H).

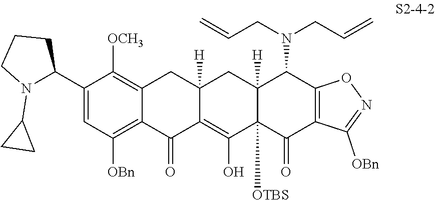 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5C5CC5)c(OC)c4C[C@H]3C[C@@H]12]

Compound S2-4-2 was prepared from S2-2-2 and S2-3 by using the General Procedure A:

¹H NMR (400 MHz, CDCl₃) δ 16.1 (s, 1H), 7.09-7.50 (m, 9H), 6.70-7.00 (m, 2H), 5.60-5.75 (m, 2H), 4.95-5.13 (m, 7H), 3.98-4.08 (m, 5H), 3.59 (s, 3H), 3.07-3.21 (m, 4H), 2.15-2.50 (m, 4H), 1.55-1.75 (m, 6H), 1.13-1.21 (m, 5H), 0.77 (s, 9H), 0.17 (s, 3H), 0.04 (s, 3H); MS (ESI) m/z 898.3 (M+H).

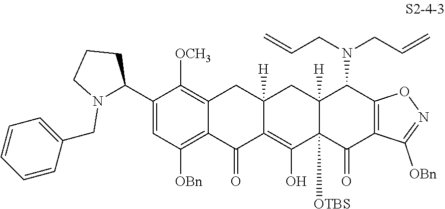 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5Cc5ccccc5)c(OC)c4C[C@H]3C[C@@H]12]

Compound S2-4-3 was prepared from S2-2-4 and S2-3 by using the General Procedure A:

¹H NMR (400 MHz, CDCl₃) δ 16.1 (s, 1H), 7.10-7.41 (m, 14H), 6.71-6.89 (m, 2H), 5.69-5.71 (m, 2H), 4.98-5.18 (m, 9H), 3.98-4.07 (m, 2H), 3.65-3.79 (m, 1H), 3.60 (s, 3H), 3.00-3.28 (m, 4H), 2.30-2.57 (m, 4H), 2.10-2.21 (m, 2H), 1.69-1.82 (m, 3H), 1.10-1.20 (m, 3H), 0.73 (s, 9H), 0.17 (s, 3H), 0.04 (s, 3H); MS (ESI) m/z 948.3 (M+H).

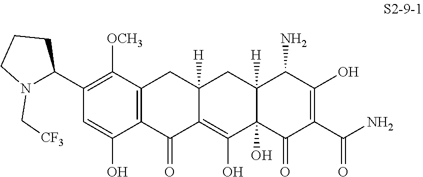 [SMILES: COc1c([C@@H]2CCCN2CC(F)(F)F)cc(O)c2c1C[C@H]1C[C@H]3[C@H](N)C(O)=C(C(N)=O)C(=O)[C@@]3(O)C(O)=C1C2=O]

Compound S2-9-1 was prepared from S2-4-1 by using the General Procedure A, C and D-1: ¹H NMR (400 MHz, CD₃OD, dihydrochloride salt) δ 7.08 (s, 1H), 3.88 (s, 1H), 3.70-3.74 (m, 1H), 3.68 (s, 3H), 3.55-3.62 (m, 2H), 3.10-3.25 (m, 2H), 2.90-3.00 (m, 1H), 2.60-2.65 (m, 1H), 2.35-2.50 (m, 3H), 2.15-2.25 (m, 3H), 2.00-2.10 (m, 1H), 1.58-1.64 (m, 1H); MS (ESI) m/z 568.3 (M+H).

The following compounds were prepared similarly to compound S2-9-1.

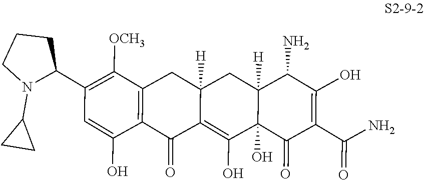 [SMILES: COc1c([C@@H]2CCCN2C2CC2)cc(O)c2c1C[C@H]1C[C@H]3[C@H](N)C(O)=C(C(N)=O)C(=O)[C@@]3(O)C(O)=C1C2=O]

S2-9-2: ¹H NMR (400 MHz, CD₃OD, dihydrochloride salt) δ 7.11 (s, 1H), 3.91 (s, 1 H), 3.77-3.83 (m, 1H), 3.70 (s, 3H), 3.50-3.57 (m, 1H), 3.21-3.27 (m, 1H), 2.87-3.00 (m, 2 H), 2.55-2.70 (m, 2H), 2.21-2.44 (m, 6H), 1.58-1.65 (m, 1H), 0.85-0.91 (m, 2H), 0.63-0.70 (m, 1H), 0.30-0.40 (m, 1H); MS (ESI) m/z 526.3 (M+H).

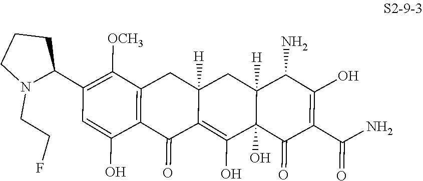 [SMILES: COc1c([C@@H]2CCCN2CCF)cc(O)c2c1C[C@H]1C[C@H]3[C@H](N)C(O)=C(C(N)=O)C(=O)[C@@]3(O)C(O)=C1C2=O]

S2-9-3: ¹H NMR (400 MHz, CD₃OD, dihydrochloride salt) δ 7.12 (s, 1H), 3.92-3.96 (m, 1H), 3.89 (s, 1H), 3.60 (s, 3H), 3.40-3.51 (m, 4H), 3.21-3.26 (m, 1H), 2.90-2.98 (m, 1 H), 2.55-2.78 (m, 2H), 2.21-2.45 (m, 6H), 1.55-1.82 (m, 2H); MS (ESI) m/z 532.3 (M+H).

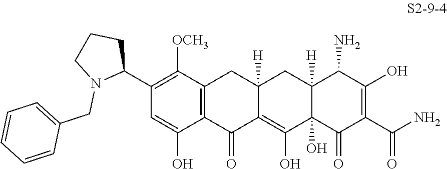 [SMILES: COc1c([C@@H]2CCCN2Cc2ccccc2)cc(O)c2c1C[C@H]1C[C@H]3[C@H](N)C(O)=C(C(N)=O)C(=O)[C@@]3(O)C(O)=C1C2=O]

S2-9-4: ¹H NMR (400 MHz, CD₃OD, dihydrochloride salt) δ 7.31-7.42 (m, 5H), 7.02 (s, 1H), 4.21-4.36 (m, 2H), 3.89 (s, 1H), 3.65 (s, 3H), 3.56-3.62 (m, 1H), 3.42-3.50 (m, 1 H), 3.18-3.22 (m, 1H), 2.89-2.97 (m, 1H), 2.55-2.65 (m, 2H), 2.21-2.49 (m, 6H), 1.55-1.65 (m, 1H); MS (ESI) m/z 576.3 (M+H).

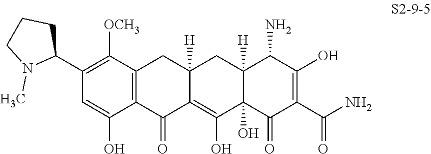 [SMILES: COc1c([C@@H]2CCCN2C)cc(O)c2c1C[C@H]1C[C@H]3[C@H](N)C(O)=C(C(N)=O)C(=O)[C@@]3(O)C(O)=C1C2=O]

Compound S2-9-5 was prepared from S2-2-5 and S2-3 by using the General Procedure E. The resulting product was treated with 0.5 N HCl in THF (83 μL of 6 N aq HCl was added to 917 μL of THF) at rt for 45 min. Then saturated NaHCO₃ was added slowly and extracted with EtOAc. The organic solution was then washed with brine, dried over Na₂SO₄, filtered and concentrated. The residue was methylated with HCHO by using General procedure B-1, followed by General Procedure A, C, D-1 to provide compound S2-9-5: ¹H NMR (400 MHz, CD₃OD, dihydrochloride salt) δ 7.10 (s, 1H), 3.90 (s, 1H), 3.78-3.85 (m, 1H), 3.68 (s, 3H), 3.32-3.38 (m, 1H), 3.21-3.28 (m, 1H), 2.90-3.00 (m, 1H), 2.79 (s, 3H), 2.55-2.68 (m, 2H), 2.21-2.41 (m, 6H), 1.55-1.65 (m, 1H); MS (ESI) m/z 500.2 (M+H).

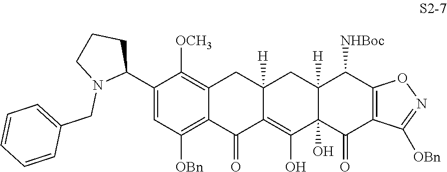 [SMILES: COc1c([C@@H]2CCCN2Cc2ccccc2)cc(OCc2ccccc2)c2c1C[C@H]1C[C@H]3[C@H](NC(=O)OC(C)(C)C)c4onc(OCc5ccccc5)c4C(=O)[C@@]3(O)C(O)=C1C2=O]

Compound S2-7 was made from S2-4-3 (3.47 g, 3.92 mmol) by using General Procedure A and C, followed by Boc protection of C-4 amino group. Thus S2-6 (R=Bn) reacted with Boc₂O (655 mg, 3.0 mmol) and TEA (0.6 mL) in DCM (30 mL) at rt for 4 h. The reaction mixture was concentrated and purified by flash column chromatography (50 g silica gel, 0-60% EtOAc/Hexane) to give the desired product S2-7 as a yellow oil (1.14 g, 33% over 4 steps).

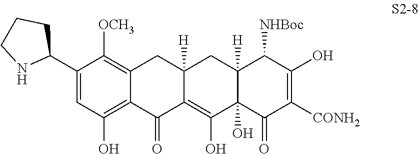 [SMILES: COc1c([C@@H]2CCCN2)cc(O)c2c1C[C@H]1C[C@H]3[C@H](NC(=O)OC(C)(C)C)C(O)=C(C(N)=O)C(=O)[C@@]3(O)C(O)=C1C2=O]

Compound 2-7 (1.14 g, 1.34 mmol) was dissolved in a mixture of 1 N aq HCl (1.34 mL, 1 eq), THF (6 mL) and CH₃OH (6 mL). Pd—C (10 wt %, 110 mg) was added in one portion. The reaction vessel was sealed and purged with hydrogen by briefly evacuating the flask followed by flushing with hydrogen gas (1 atm). The reaction mixture was stirred under a hydrogen atmosphere (1 atm) at rt for two overnights. The reaction was filtered through a small Celite pad. The cake was washed with CH₃OH. The filtrate was concentrated. The residue was re-slurried from MTBE to give product S2-8 as a yellow solid, which was used for the following reductive alkylation reactions without further purification: MS (ESI) m/z 586.2 (M+H).

General Procedure F (reductive alkylation): To the solution of pyrrolidine S2-8 (1 eq) in CH₃OH (1 mL) at 0° C., aldehyde/ketone (4 eq), HOAc (4 eq) and NaBH(OAc)₃ (4 eq) were added. The resulting reaction mixture was stirred at 0° C. for 1 h or longer which monitored by LC-MS.

General Procedure G (deprotection of Boc): After the completeness of reductive amination, concentrated HCl (0.5 mL) was added. The resulted mixture was stirred at room temperature for 0.5 h. The organic solvents were removed under reduced pressure and preparative HPLC afforded the desired products as yellow solids.

NOTE: The ketones and 4-pyridinecarboxaldehyde required much longer time for reductive amination.

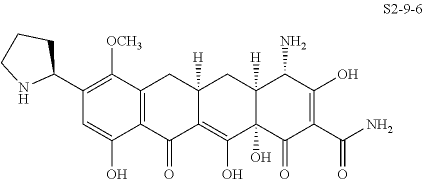 [SMILES: COc1c([C@@H]2CCCN2)cc(O)c2c1C[C@H]1C[C@H]3[C@H](N)C(O)=C(C(N)=O)C(=O)[C@@]3(O)C(O)=C1C2=O]

Compound S2-9-6 was prepared from compound S2-8 by using General Procedure G: ¹H NMR (400 MHz, CD₃OD, dihydrochloride salt) δ 6.96 (s, 1H), 3.91 (s, 1H), 3.71 (s, 3H), 3.40-3.47 (m, 1H), 3.30-3.35 (m, 1H), 3.20-3.25 (m, 1H), 2.88-2.95 (m, 1H), 2.63-2.67 (m, 1H), 2.39-2.50 (m, 2H), 2.15-2.30 (m, 5H), 1.58-1.65 (m, 1H); MS (ESI) m/z 486.2 (M+H).

The following compounds were prepared from compound S2-8 by using General Procedure F and G.

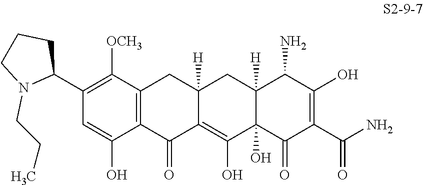 [SMILES: CCCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-7: ¹H NMR (400 MHz, CD₃OD, dihydrochloride salt) δ 7.09 (s, 1H), 3.89 (s, 1 H), 3.78-3.88 (m, 1H), 3.67 (s, 3H), 3.34-3.38 (m, 1H), 3.22-3.28 (m, 1H), 2.94-3.05 (m, 4 H), 2.55-2.65 (m, 2H), 2.21-2.49 (m, 5H), 1.55-1.82 (m, 3H), 0.88-0.94 (t, J=8.0 Hz, 3H); MS (ESI) m/z 528.2 (M+H).

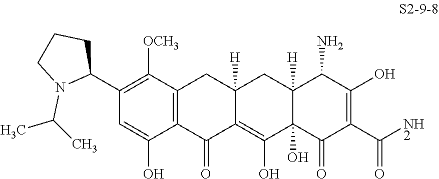 [SMILES: COc1c([C@@H]2CCCN2C(C)C)cc(O)c2c1C[C@H]1C[C@H]3[C@H](N)C(O)=C(C(N)=O)C(=O)[C@@]3(O)C(O)=C1C2=O]

S2-9-8: ¹H NMR (400 MHz, CD₃OD, dihydrochloride salt) δ 7.19 (s, 1H), 3.90 (s, 1 H), 3.68 (s, 3H), 3.39-3.48 (m, 2H), 3.27-3.11 (m, 4H), 2.89-2.96 (m, 1H), 2.55-2.71 (m, 2 H), 2.37-2.45 (m, 1H), 2.21-2.31 (m, 3H), 1.55-1.65 (m, 1H), 1.28 (t, J=6.0 Hz, 6H); MS (ESI) m/z 528.3 (M+H).

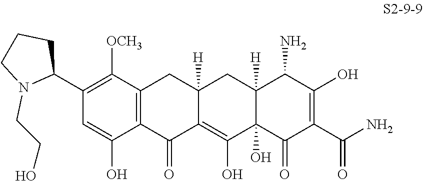 [SMILES: COc1c([C@@H]2CCCN2CCO)cc(O)c2c1C[C@H]1C[C@H]3[C@H](N)C(O)=C(C(N)=O)C(=O)[C@@]3(O)C(O)=C1C2=O]

S2-9-9: ¹H NMR (400 MHz, CD₃OD, dihydrochloride salt) δ 7.13 (s, 1H), 3.92-3.97 (m, 1H), 3.90 (s, 1H), 3.72-3.80 (m, 3H), 3.70 (s, 3H), 3.37-3.41 (m, 1H), 3.15-3.20 (m, 3 H), 2.90-3.00 (m, 1H), 2.55-2.70 (m, 2H), 2.20-2.41 (m, 5H), 1.57-1.67 (m, 1H); MS (ESI) m/z 530.2 (M+H).

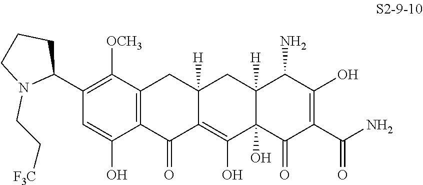 [SMILES: COc1c([C@@H]2CCCN2CCC(F)(F)F)cc(O)c2c1C[C@H]1C[C@H]3[C@H](N)C(O)=C(C(N)=O)C(=O)[C@@]3(O)C(O)=C1C2=O]

S2-9-10: ¹H NMR (400 MHz, CD₃OD, dihydrochloride salt) δ 7.11 (s, 1H), 3.85-3.91 (m, 2H), 3.68 (s, 3H), 3.38-3.48 (m, 4H), 2.90-3.00 (m, 1H), 2.73-2.81 (m, 1H), 2.68-2.78 (m, 3H), 2.23-2.41 (m, 5H), 1.58-1.65 (m, 1H), 1.26-1.31 (m, 1H); MS (ESI) m/z 582.3 (M+H).

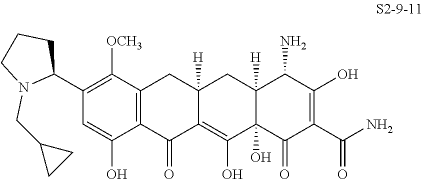 [SMILES: COc1c([C@@H]2CCCN2CC2CC2)cc(O)c2c1C[C@H]1C[C@H]3[C@H](N)C(O)=C(C(N)=O)C(=O)[C@@]3(O)C(O)=C1C2=O]

S2-9-11: ¹H NMR (400 MHz, CD₃OD, dihydrochloride salt) δ 7.15 (s, 1H), 3.91-4.00 (m, 1H), 3.90 (s, 1H), 3.69 (s, 3H), 3.40-3.50 (m, 1H), 3.20-3.41 (m, 4H), 2.93-3.04 (m, 1 H), 2.56-2.71 (m, 2H), 2.19-2.51 (m, 5H), 1.54-1.65 (m, 1H), 0.98-1.07 (m, 1H), 0.58-0.77 (m, 2H), 0.32-0.40 (m, 1H), 0.20-0.27 (m, 1H); MS (ESI) m/z 540.3 (M+H).

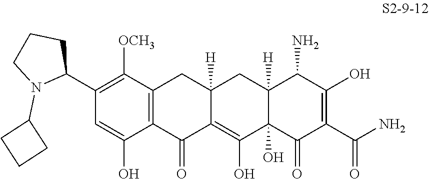 [SMILES: COc1c([C@@H]2CCCN2C2CCC2)cc(O)c2c1C[C@H]1C[C@H]3[C@H](N)C(O)=C(C(N)=O)C(=O)[C@@]3(O)C(O)=C1C2=O]

S2-9-12: ¹H NMR (400 MHz, CD₃OD, dihydrochloride salt) δ 7.17 (s, 1H), 3.83-3.91 (m, 2H), 3.70-3.75 (m, 2H), 3.68 (s, 3H), 3.20-3.24 (m, 1H), 2.88-2.95 (m, 1H), 2.53-2.78 (m, 2H), 2.21-2.42 (m, 8H), 1.84-2.93 (m, 1H), 1.58-1.80 (m, 4H); MS (ESI) m/z 540.3 (M+H)

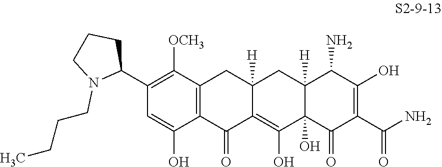 [SMILES: CCCCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-13: ¹H NMR (400 MHz, CD₃OD, dihydrochloride salt) δ 7.13 (s, 1H), 3.92 (s, 1 H), 3.82-3.89 (m, 1H), 3.70 (s, 3H), 3.50-3.57 (m, 1H), 3.03-3.12 (m, 2H), 2.91-3.00 (m, 1 H), 2.55-2.71 (m, 3H), 2.21-2.45 (m, 5H), 1.55-1.71 (m, 3H), 1.25-1.37 (m, 3H), 0.88-0.93 (m, 3H); MS (ESI) m/z 542.3 (M+H).

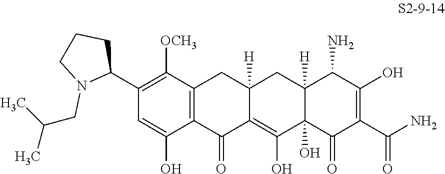 [SMILES: COc1c([C@@H]2CCCN2CC(C)C)cc(O)c2c1C[C@H]1C[C@H]3[C@H](N)C(O)=C(C(N)=O)C(=O)[C@@]3(O)C(O)=C1C2=O]

S2-9-14: ¹H NMR (400 MHz, CD₃OD, dihydrochloride salt) δ 7.17 (s, 1H), 3.88-3.95 (m, 2H), 3.68 (s, 3H), 3.45-3.51 (m, 1H), 3.23-3.30 (m, 4H), 2.82-3.05 (m, 3H), 2.55-2.70 (m, 2H), 2.23-2.45 (m, 3H), 1.93-2.00 (m, 1H), 1.57-1.63 (m, 1H), 0.89-0.95 (m, 6H); MS (ESI) m/z 542.3 (M+H).

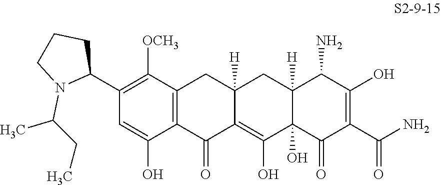 [SMILES: CCC(C)N1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-15: ¹H NMR (400 MHz, CD₃OD, dihydrochloride salt, two isomers) δ 7.11+7.13 (s, 1H), 3.89 (s, 1H), 3.68 (s, 3H), 3.40-3.48 (m, 1H), 3.10-3.18 (m, 1H), 2.90-3.00 (m, 1 H), 2.52-2.63 (m, 2H), 2.38-2.48 (m, 1H), 2.20-2.31 (m, 5H), 1.80-1.90 (m, 1H), 1.56-1.62 (m, 2H), 1.25-1.30 (m, 5H), 0.88-0.93 (m, 3H); MS (ESI) m/z 583.3 (M+H).

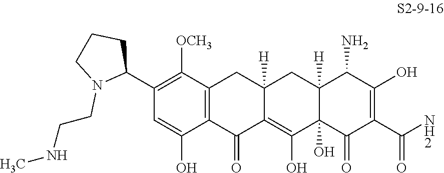 [SMILES: CNCCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-16: ¹H NMR (400 MHz, CD₃OD, trihydrochloride salt) δ 7.30 (s, 1H), 3.95-4.03 (m, 1H), 3.90 (s, 1H), 3.70 (s, 3H), 3.39-3.51 (m, 5H), 3.21-3.25 (m, 1H), 2.94-3.02 (m, 1 H), 2.58-2.69 (m, 5H), 2.31-2.43 (m, 5H), 2.20-2.27 (m, 1H), 1.55-1.65 (m, 1H); MS (ESI) m/z 543.3 (M+H).

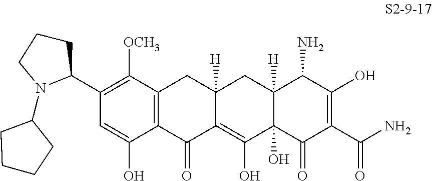 [SMILES: COc1c([C@@H]2CCCN2C2CCCC2)cc(O)c2c1C[C@H]1C[C@H]3[C@H](N)C(O)=C(C(N)=O)C(=O)[C@@]3(O)C(O)=C1C2=O]

S2-9-17: ¹H NMR (400 MHz, CD₃OD, dihydrochloride salt) δ 7.17 (s, 1H), 3.92 (s, 1 H), 3.75-3.81 (m, 1H), 3.68 (s, 3H), 3.41-3.50 (m, 1H), 2.90-3.00 (m, 1H), 2.58-2.70 (m, 2 H), 2.20-2.42 (m, 6H), 2.07-2.14 (m, 1H), 1.50-1.90 (m, 8H), 1.27-1.40 (m, 2H); MS (ESI) m/z 554.3 (M+H).

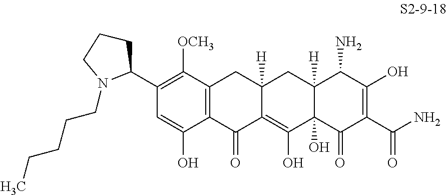 [SMILES: CCCCCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-18: ¹H NMR (400 MHz, CD₃OD, dihydrochloride salt) δ 7.12 (s, 1H), 3.85-3.91 (m, 2H), 3.72-3.75 (m, 2H), 3.69 (s, 3H), 3.39-3.43 (m, 5H), 2.75-3.00 (m, 3H), 2.58-2.69 (m, 4H), 2.21-2.45 (m, 6H), 1.58-1.67 (m, 2H), 1.27-1.31 (m, 1H); MS (ESI) m/z 556.3 (M+H).

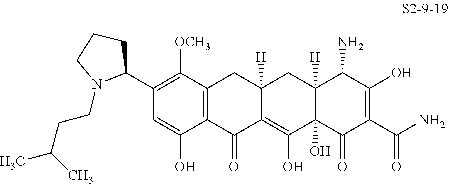 [SMILES: COc1c([C@@H]2CCCN2CCC(C)C)cc(O)c2c1C[C@H]1C[C@H]3[C@H](N)C(O)=C(C(N)=O)C(=O)[C@@]3(O)C(O)=C1C2=O]

S2-9-19: ¹H NMR (400 MHz, CD₃OD, dihydrochloride salt) δ 7.14 (s, 1H), 3.91 (s, 1 H), 3.81-3.88 (m, 1H), 3.69 (s, 3H), 3.25-3.50 (m, 3H), 3.05-3.15 (m, 2H), 2.90-3.00 (m, 1 H), 2.55-2.70 (m, 2H), 2.22-2.58 (m, 5H), 1.47-1.70 (m, 4H), 0.87 (t, J=6.0 Hz, 6H); MS (ESI) m/z 556.3 (M+H).

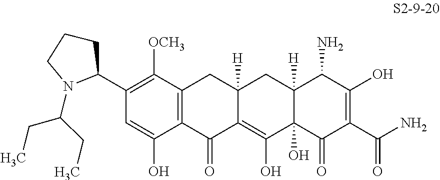 [SMILES: CCC(CC)N1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-20: ¹H NMR (400 MHz, CD₃OD, dihydrochloride salt) δ 7.13 (s, 1H), 3.78 (s, 1 H), 3.69 (s, 3H), 3.41-3.50 (m, 2H), 2.80-2.92 (m, 3H), 2.50-2.61 (m, 3H), 2.18-2.33 (m, 5 H), 1.61-1.88 (m, 4H), 1.27-1.31 (m, 1H), 0.85-0.97 (m, 6H); MS (ESI) m/z 556.3 (M+H).

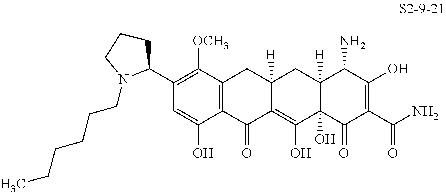 [SMILES: CCCCCCN1CCC[C@H]1c1cc(O)c2c(c1OC)C[C@H]1C[C@H]3[C@H](N)C(O)=C(C(N)=O)C(=O)[C@@]3(O)C(O)=C1C2=O]

S2-9-21: ¹H NMR (400 MHz, CD₃OD, dihydrochloride salt) δ 7.11 (s, 1H), 3.91 (s, 1 H), 3.82-3.90 (m, 1H), 3.68 (s, 3H), 3.02-3.10 (m, 2H), 2.90-3.00 (m, 1H), 2.55-2.70 (m, 2 H), 2.21-2.45 (m, 6H), 1.55-1.70 (m, 4H), 1.18-1.31 (m, 7H), 0.83-0.91 (m, 3H); MS (ESI) m/z 570.4 (M+H).

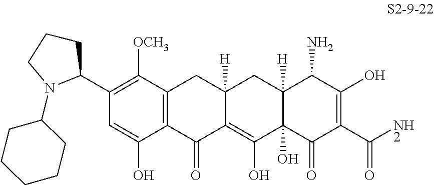 [SMILES: COc1c([C@@H]2CCCN2C2CCCCC2)cc(O)c2c1C[C@H]1C[C@H]3[C@H](N)C(O)=C(C(N)=O)C(=O)[C@@]3(O)C(O)=C1C2=O]

S2-9-22: ¹H NMR (400 MHz, CD₃OD, dihydrochloride salt) δ 7.11 (s, 1H), 3.89 (s, 1 H), 3.68 (s, 3H), 3.45-3.51 (m, 1H), 3.07-3.12 (m, 1H), 2.90-3.00 (m, 1H), 2.55-2.67 (m, 2 H), 2.38-2.43 (m, 1H), 2.20-2.31 (m, 5H), 2.05-2.11 (m, 1H), 1.88-2.00 (m, 3H), 1.59-1.67 (m, 3H), 1.11-1.42 (m, 6H); MS (ESI) m/z 568.3 (M+H).

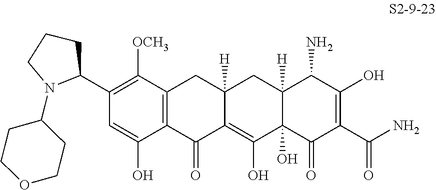 [SMILES: COc1c([C@@H]2CCCN2C2CCOCC2)cc(O)c2c1C[C@H]1C[C@H]3[C@H](N)C(O)=C(C(N)=O)C(=O)[C@@]3(O)C(O)=C1C2=O]

S2-9-23: ¹H NMR (400 MHz, CD₃OD, dihydrochloride salt) δ 7.18 (s, 1H), 3.88-4.01 (m, 2H), 3.70-3.75 (m, 2H), 3.68 (s, 3H), 3.40-3.51 (m, 2H), 3.30-3.38 (m, 2H), 2.90-3.00 (m, 1H), 2.59-2.70 (m, 2H), 2.20-2.42 (m, 6H), 1.96-2.02 (m, 1H), 1.85-1.92 (m, 1H), 1.58-1.78 (m, 4H); MS (ESI) m/z 570.3 (M+H).

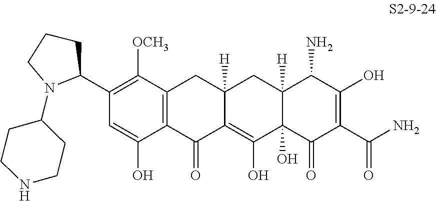 [SMILES: COc1c([C@@H]2CCCN2C2CCNCC2)cc(O)c2c1C[C@H]1C[C@H]3[C@H](N)C(O)=C(C(N)=O)C(=O)[C@@]3(O)C(O)=C1C2=O]

S2-9-24: ¹H NMR (400 MHz, CD₃OD, trihydrochloride salt) δ 7.25 (s, 1H), 3.91 (s, 1 H), 3.73-3.81 (m, 1H), 3.68 (s, 3H), 3.45-3.61 (m, 5H), 2.98-3.11 (m, 3H), 2.69-2.70 (m, 2 H), 2.21-2.42 (m, 8H), 1.83-2.05 (m, 2H), 1.57-1.65 (m, 1H); MS (ESI) m/z 569.3 (M+H).

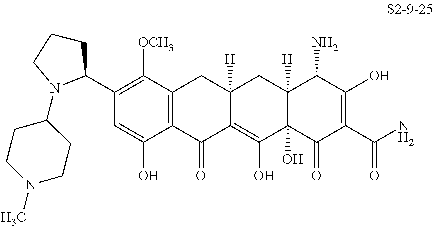 [SMILES: COc1c([C@@H]2CCCN2C2CCN(C)CC2)cc(O)c2c1C[C@H]1C[C@H]3[C@H](N)C(O)=C(C(N)=O)C(=O)[C@@]3(O)C(O)=C1C2=O]

S2-9-25: ¹H NMR (400 MHz, CD₃OD, trihydrochloride salt) δ 7.20 (s, 1H), 3.89 (s, 1 H), 3.72-3.80 (m, 1H), 3.70 (s, 3H), 3.53-3.61 (m, 5H), 3.05-3.18 (m, 3H), 2.83 (s, 3H), 2.60-2.70 (m, 2H), 2.23-2.41 (m, 8H), 1.93-2.15 (m, 2H), 1.58-1.63 (m, 1H); MS (ESI) m/z 583.3 (M+H).

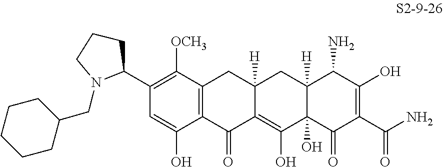 [SMILES: COc1c([C@@H]2CCCN2CC2CCCCC2)cc(O)c2c1C[C@H]1C[C@H]3[C@H](N)C(O)=C(C(N)=O)C(=O)[C@@]3(O)C(O)=C1C2=O]

S2-9-26: ¹H NMR (400 MHz, CD₃OD, dihydrochloride salt) δ 7.16 (s, 1H), 3.91 (s, 1 H), 3.69 (s, 3H), 3.21-3.51 (m, 5H), 2.88-3.06 (m, 3H), 2.52-2.72 (m, 2H), 2.21-2.45 (m, 5 H), 1.77-1.85 (m, 1H), 1.50-1.72 (m, 5H), 1.05-1.30 (m, 3H), 0.78-0.96 (m, 2H); MS (ESI) m/z 582.4 (M+H).

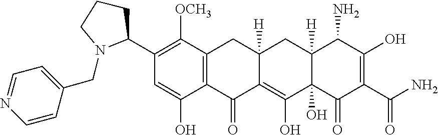 [SMILES: COc1c([C@@H]2CCCN2Cc2ccncc2)cc(O)c2c1C[C@H]1C[C@H]3[C@H](N)C(O)=C(C(N)=O)C(=O)[C@@]3(O)C(O)=C1C2=O]

S2-9-27: ¹H NMR (400 MHz, CD₃OD, trihydrochloride salt) δ 8.80-8.89 (m, 2H), 8.12-8.20 (m, 2H), 7.22 (s, 1H), 4.58-4.63 (m, 2H), 3.88-3.95 (m, 2H), 3.65 (s, 3H), 3.47-3.55 (m, 1H), 3.21-3.30 (m, 1H), 3.03-3.11 (m, 1H), 2.85-2.95 (m, 1H), 2.58-2.77 (m, 2H), 2.25-2.41 (m, 5H), 1.50-1.61 (m, 1H); MS (ESI) m/z 577.3 (M+H).

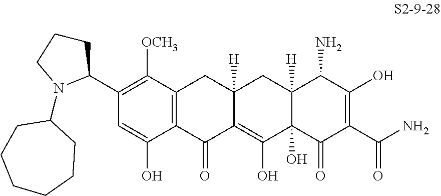 [SMILES: COc1c([C@@H]2CCCN2C2CCCCCC2)cc(O)c2c1C[C@H]1C[C@H]3[C@H](N)C(O)=C(C(N)=O)C(=O)[C@@]3(O)C(O)=C1C2=O]

S2-9-28: ¹H NMR (400 MHz, CD₃OD, dihydrochloride salt) δ 7.11 (s, 1H), 3.90 (s, 1 H), 3.69 (s, 3H), 3.39-3.45 (m, 2H), 3.15-3.20 (m, 1H), 2.93-3.00 (m, 1H), 2.38-2.61 (m, 4 H), 2.20-2.31 (m, 5H), 1.95-2.01 (m, 3H), 1.60-1.80 (m, 5H), 1.37-1.51 (m, 7H); MS (ESI) m/z 582.3 (M+H).

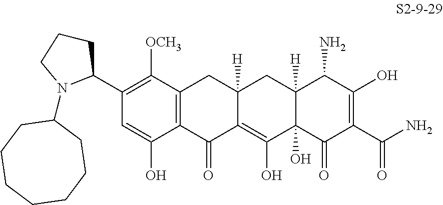 [SMILES: COc1c([C@@H]2CCCN2C2CCCCCCC2)cc(O)c2c1C[C@H]1C[C@H]3[C@H](N)C(O)=C(C(N)=O)C(=O)[C@@]3(O)C(O)=C1C2=O]

S2-9-29: ¹H NMR (400 MHz, CD₃OD, dihydrochloride salt) δ 7.12 (s, 1H), 3.73-3.78 (m, 1H), 3.68 (s, 3H), 2.78-2.83 (m, 2H), 2.41-2.55 (m, 3H), 2.25-2.31 (m, 6H), 2.11-2.18 (m, 1H), 1.95-2.01 (m, 1H), 1.70-1.80 (m, 4H), 1.45-1.52 (m, 8H), 1.25-1.30 (m, 3H); MS (ESI) m/z 596.3 (M+H).

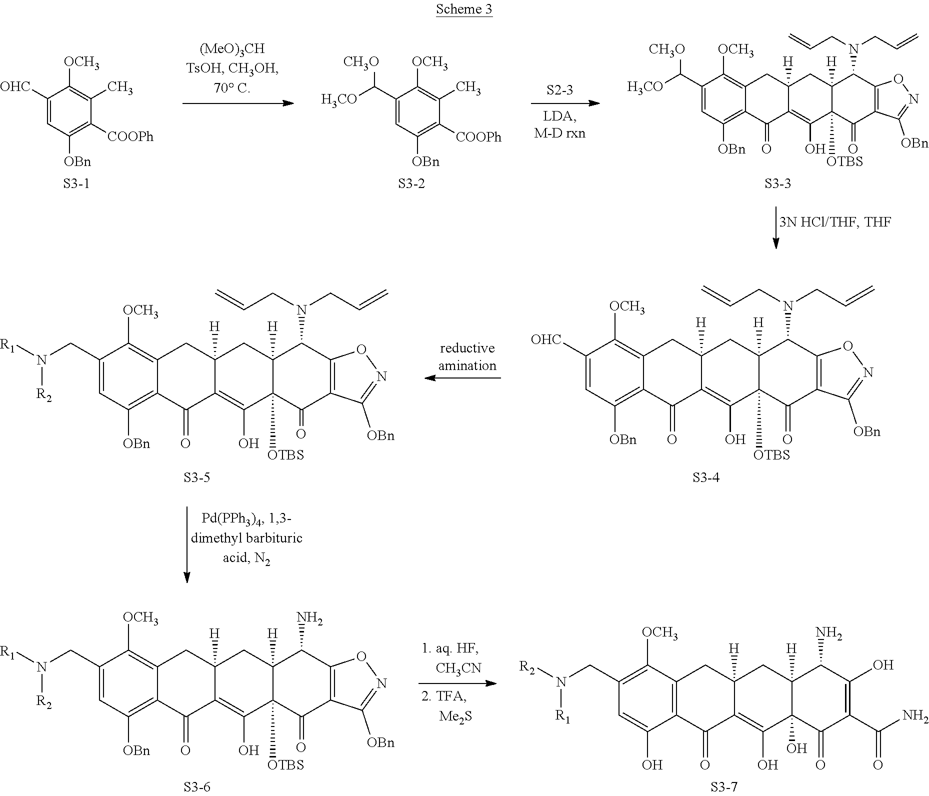

The following compounds were prepared per Scheme 3.

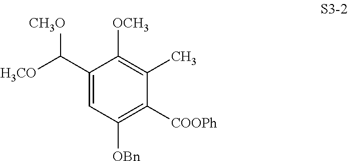

Compound S3-1 (1.88 g, 5.0 mmol, 1 eq, prepared per literature procedures: Org. Process Res. Dev., 2016, 20 (2), 284-296) was dissolved in CH₃OH (10 mL), trimethyl orthoformate (1.10 mL, 10.05 mmol, 2 eq) and p-toluenesulfonic acid monohydrate (29 mg, 0.152 mmol, 0.03 eq) were added. The reaction mixture was stirred at 70° C. for 24 h. Saturated NaHCO₃ and EtOAc were added. The organic phase was separated, concentrated by rotovap and purified by flash column chromatography to afford the desired product S3-2 as a yellow oil (2.03 g, 96%):

¹H NMR (400 MHz, CDCl₃) δ 7.23-7.45 (m, 8H), 7.05-7.11 (m, 3H), 5.61 (s, 1H), 5.15 (s, 2 H), 3.76 (s, 3H), 3.36 (s, 6H), 2.39 (s, 3H); MS (ESI) m/z 423.2 (M+H).

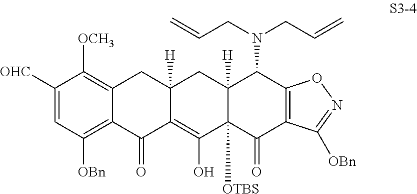 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc(C=O)c(OC)c4C[C@H]3C[C@@H]12]

Compound S3-4 was prepared from S3-2 with enone S2-3 by using General Procedure E, followed by acid treatment. The M-D product S3-3 (1.30 g, 1.51 mmol, 1 eq) was dissolved in THF (20 mL). Then 3 N HCl/THF (4 mL) was added to make the final aqueous HCl concentration to 0.5 M. The reaction mixture was stirred at room temperature for 2 h. Saturated NaHCO₃ and EtOAc were added. The organic phase was concentrated by rotovap, and the residue was purified by flash column chromatography to afford the desired product S3-4 as a yellow oil (1.15 g, 47% over 2 steps): ¹H NMR (400 MHz, CDCl₃) δ 15.89 (s, 1H), 10.35 (s, 1H), 7.31-7.52 (m, 11H), 5.78-5.85 (m, 2H), 5.35 (s, 2H), 5.08-5.25 (m, 5H), 4.06-4.11 (m, 1H), 3.86 (s, 3H), 3.18-3.38 (m, 5H), 2.41-2.63 (m, 4H), 0.81 (s, 9H), 0.25 (s, 3H), 0.12 (s, 3H); MS (ESI) m/z 817.3 (M+H).

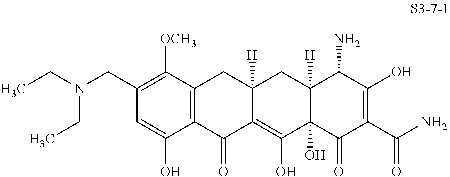 [SMILES: CCN(CC)Cc1cc(O)c2c(c1OC)C[C@H]1C[C@H]3[C@H](N)C(O)=C(C(N)=O)C(=O)[C@@]3(O)C(O)=C1C2=O]

Compound S3-7-1 was prepared from aldehyde S3-4 and diethylamine by using General Procedure B-1, followed by General Procedures A, C and D-1: ¹H NMR (400 MHz, CD₃OD, dihydrochloride salt) δ 7.01 (s, 1H), 4.34 (d, J=8.0, 1H), 4.30 (d, J=8.0, 1H), 3.89 (s, 1H), 3.73 (s, 3H), 3.13-3.27 (m, 5H), 2.90-2.98 (m, 1H), 2.62-2.67 (m, 1H), 2.37-2.45 (m, 1H), 2.20-2.28 (m, 1H), 1.59-1.65 (m, 1H), 1.30-1.42 (m, 6H); MS (ESI) m/z 502.4 (M+H).

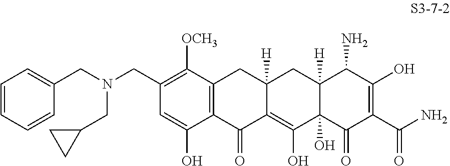 [SMILES: COc1c(CN(Cc2ccccc2)CC2CC2)cc(O)c2c1C[C@H]1C[C@H]3[C@H](N)C(O)=C(C(N)=O)C(=O)[C@@]3(O)C(O)=C1C2=O]

Compound S3-7-2 was prepared from aldehyde S3-4 and benzylamine by using General Procedure B-1, followed by reacting with cyclopropanecarboxaldehyde using General Procedure B-1 again and then A, C and D-1: ¹H NMR (400 MHz, CD₃OD, 2 hydrochloride salt, two rotamers) δ 7.40-7.60 (m, 5H), 6.83+6.93 (s, 1H), 4.48-4.68 (m, 2H), 4.21-4.49 (m, 2H), 3.88+3.53 (s, 3H), 3.02-3.18 (m, 3H), 2.88-2.97 (m, 1H), 2.60-2.68 (m, 1H), 2.19-2.38 (m, 2H), 1.51-1.61 (m, 1H), 1.18-1.27 (m, 1H), 0.70-0.85 (m, 2H), 0.38-0.45 (m, 2H); MS (ESI) m/z 590.3 (M+H).

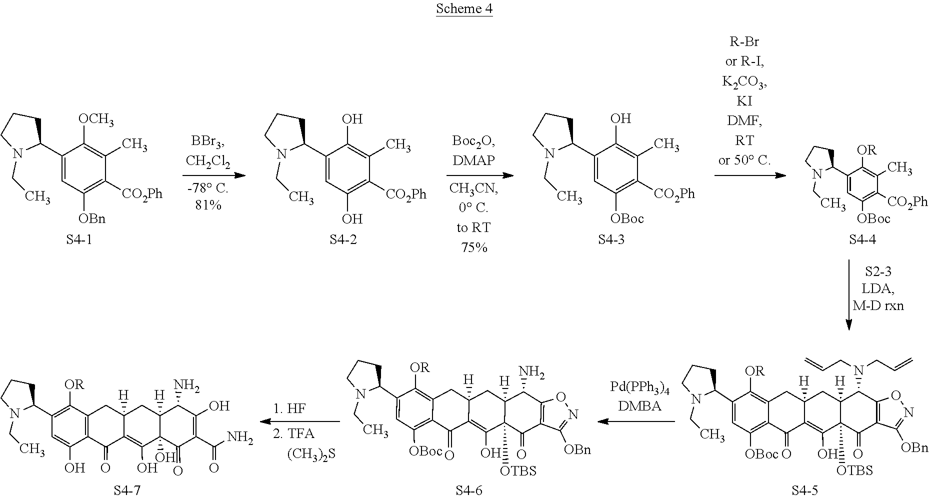

The following compounds were prepared per Scheme 4.

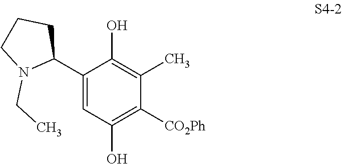

Compound S4-1 (504 mg, 1.13 mmol, 1 eq, prepared per literature procedures: Org.

Process Res. Dev., 2016, 20 (2), 284-296) was dissolved in CH₂Cl₂ (3 mL) and cooled down to −78° C. under N₂, then BBr₃ solution (1.0 M in CH₂Cl₂, 3.4 mL, 3.4 mmol, 3 eq) was added dropwise during 5 min. The resulted yellow mixture was stirred at −78° C. for 4.5 h and carefully quenched by CH₃OH (2 mL). CH₂Cl₂ (40 mL) was added to the dark solution and washed with saturated NaHCO₃. The organic phase was concentrated by rotovap. The residue was purified by flash column chromatography (0→55% EtOAc/hexane) to afford the desired product S4-2 as a yellow oil (312 mg, 81%): ¹H NMR (400 MHz, CDCl₃) δ 11.65 (br s, 1H), 10.25 (br s, 1H), 7.39-7.47 (m, 2H), 7.15-7.30 (m, 3H), 6.66 (s, 1H), 3.39-3.55 (m, 2H), 3.79-3.88 (m, 1H), 2.58 (s, 3H), 2.20-2.43 (m, 3H), 1.90-2.11 (m, 3H), 1.10-1.23 (m, 3H); MS (ESI) m/z 342.2 (M+H).

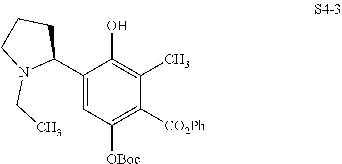

Compound S4-2 (141 mg, 0.413 mmol, 1 eq) and 4-dimethylaminopyridine (DMAP, 8 mg, 0.066 mmol, 0.16 eq) were dissolved in CH₃CN (1 mL) and the resulting solution was cooled down to 0° C. A solution of di-tert-butyl dicarbonate (Boc₂O, 90 mg, 0.413 mmol, 1 eq) in CH₃CN (1.0 mL) was added slowly. The reaction mixture was warmed up to room temperature and the white precipitates appeared. After stirring overnight, CH₂Cl₂ (100 mL) was added and washed by saturated NaHCO₃. The organic phase was concentrated by rotovap and purified by flash column chromatography (0→50% EtOAc/hexane) to afford the desired product S4-3 as a white solid (136 mg, 75%): ¹H NMR (400 MHz, CDCl₃) δ 11.62 (br s, 1H), 7.38-7.45 (m, 2H), 7.21-7.30 (m, 3H), 6.75 (s, 1H), 3.50-3.55 (m, 1H), 3.37-3.42 (m, 1H), 2.88-2.95 (m, 1H), 2.35 (s, 3H), 2.17-2.31 (m, 3H), 1.86-2.00 (m, 3H), 1.42 (s, 9H), 1.08-1.14 (m, 3H); MS (ESI) m/z 442.2 (M+H).

[NOTE: this product has low solubility in DCM, EtOAc and CH₃OH and should be able to be purified from simple recrystallization.]

General Procedure H (C₇—OH alkylation): Phenol S4-3 and K₂CO₃ were added into DMF, then R—Br/KI or R—I was added and the resulted mixture was stirred at room temperature or 50° C. for indicated hours. EtOAc was added and washed with brine solution. The organic phase was concentrated by rotovap. The residue was purified through flash column chromatography to afford the desired products S4-4-1 to S4-4-5 as colorless oils.

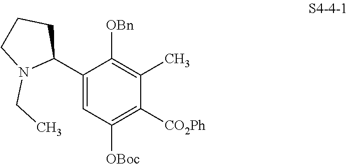

Phenol S4-3 (125 mg, 0.283 mmol, 1 eq) was treated with K₂CO₃ (60 mg, 0.434 mmol, 1.5 eq), KI (5 mg, 0.030 mmol, 0.1 eq), and BnBr (0.031 mL, 0.286 mmol, 1 eq) in DMF (2 mL) at room temperature for 18 h to give product S4-4-1 (111 mg, 74%): ¹H NMR (400 MHz, CDCl₃) δ 7.21-7.51 (m, 11H), 4.81 (s, 2H), 3.65-3.71 (m, 1H), 3.30-3.39 (m, 1 H), 2.59-2.65 (m, 1H), 2.46 (s, 3H), 2.05-2.21 (m, 2H), 1.57-1.95 (m, 3H), 1.42 (s, 9H), 1.20-1.25 (m, 1H), 1.00-1.09 (m, 3H); MS (ESI) m/z 532.3 (M+H).

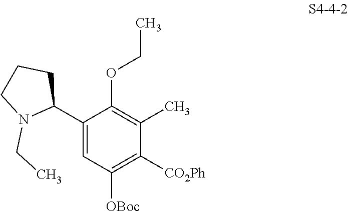

Phenol S4-3 (88 mg, 0.199 mmol, 1 eq) was treated with K₂CO₃ (41 mg, 0.297 mmol, 1.5 eq), KI (3 mg, 0.018 mmol, 0.1 eq), and C₂H₅Br (0.030 mL, 0.402 mmol, 2 eq) in DMF (2 mL) 50° C. for 23 h to give product S4-4-2 (81 mg, 86%): ¹H NMR (400 MHz, CDCl₃) δ 7.35-7.43 (m, 2H), 7.20-7.30 (m, 4H), 4.06-4.12 (m, 1H), 3.75-3.82 (m, 2H), 3.57-3.65 (m, 1H), 3.31-3.38 (m, 1H), 2.55-2.62 (m, 1H), 2.40 (s, 3H), 2.15-2.25 (m, 2H), 1.78-1.85 (m, 2H), 1.53-1.62 (m, 2H), 1.41 (s, 9H), 1.20-1.25 (m, 2H), 0.97-1.05 (m, 3H); MS (ESI) m/z 470.3 (M+H).

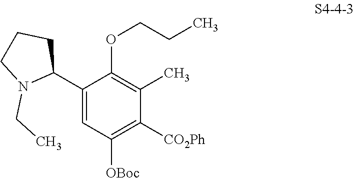

Phenol S4-3 (89 mg, 0.202 mmol, 1 eq) was treated with K₂CO₃ (41 mg, 0.297 mmol, 1.5 eq), and n-C₃H₇I (0.039 mL, 0.401 mmol) in DMF (2 mL) at 50° C. for 24 h to give product S4-4-3 (98 mg, 90%): ¹H NMR (400 MHz, CDCl₃) δ 7.38-7.45 (m, 2H), 7.21-7.28 (m, 4H), 4.05-4.11 (m, 1H), 3.70-3.81 (m, 2H), 3.30-3.37 (m, 1H), 2.56-2.63 (m, 1H), 2.40 (s, 3H), 2.15-2.22 (m, 2H), 1.78-1.85 (m, 2H), 1.55-1.66 (m, 2H), 1.41 (s, 9H), 1.20-1.27 (m, 2H), 1.00-1.15 (m, 6H); MS (ESI) m/z 484.3 (M+H).

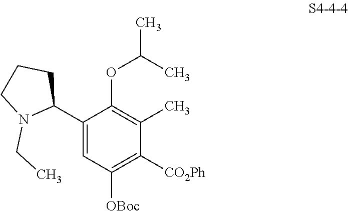

Phenol S4-3 (220 mg, 0.499 mmol, 1 eq) was treated with K₂CO₃ (104 mg, 0.753 mmol, 1.5 eq), KI (9 mg, 0.054 mmol, 0.1 eq), and (CH₃)₂CHBr (0.470 mL, 5.00 mmol, 10 eq) in DMF at 50° C. for 40 h to give product S4-4-4 (133 mg, 55%): ¹H NMR (400 MHz, CDCl₃) δ 7.38-7.45 (m, 2H), 7.21-7.28 (m, 4H), 4.07-4.16 (m, 2H), 3.65-3.71 (m, 1H), 3.30-3.40 (m, 1H), 2.52-2.61 (m, 1H), 2.40 (s, 3H), 2.15-2.26 (m, 2H), 1.78-1.95 (m, 2H), 1.50-1.60 (m, 2H), 1.42 (s, 9H), 1.20-1.35 (m, 5H), 0.98-1.05 (m, 3H); MS (ESI) m/z 484.3 (M+H).

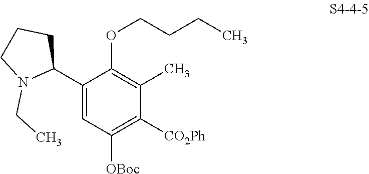

Phenol S4-3 (89 mg, 0.202 mmol, 1 eq) was treated with K₂CO₃ (41 mg, 0.297 mmol, 1.5 eq), KI (3 mg, 0.018 mmol, 0.1 eq), and n-C₄H₉Br (0.193 mL, 1.79 mmol, 9 eq) in DMF (2 mL) at 50° C. for 53 h to give product S4-4-5 (75 mg, 75%): ¹H NMR (400 MHz, CDCl₃) δ 7.21-7.43 (m, 6H), 4.08-4.13 (m, 2H), 3.69-3.75 (m, 2H), 3.30-3.36 (m, 1H), 2.56-2.63 (m, 1H), 2.40 (s, 3H), 2.15-2.22 (m, 2H), 1.75-1.82 (m, 2H), 1.50-1.55 (m, 2H), 1.43 (s, 9H), 1.20-1.27 (m, 3H), 0.97-1.05 (m, 6H); MS (ESI) m/z 498.3 (M+H).

The following compounds were prepared from the corresponding left-hand sides S4-4 and enone S2-3 by using the General Procedures E, A, C and D-1.

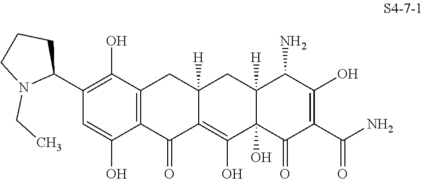 [SMILES: CCN1CCC[C@H]1c1cc(O)c2c(c1O)C[C@H]1C[C@H]3[C@H](N)C(O)=C(C(N)=O)C(=O)[C@@]3(O)C(O)=C1C2=O]

Compound S4-7-1 was isolated as a side product along with S4-7-2 in the final step when using S4-4-1 as the left-hand side: ¹H NMR (400 MHz, CD₃OD, dihydrochloride salt) δ 7.47-7.51 (m, 1H), 6.91 (s, 1H), 4.69-4.76 (m, 1H), 3.82-3.90 (m, 2H), 3.11-3.20 (m, 3H), 2.90-2.98 (m, 1H), 2.62-2.67 (m, 1H), 2.45-2.52 (m, 1H), 2.20-2.30 (m, 5H), 1.55-1.62 (m, 1H), 1.25 (t, J=5.6 Hz, 3H); MS (ESI) m/z 5003 (M+H)

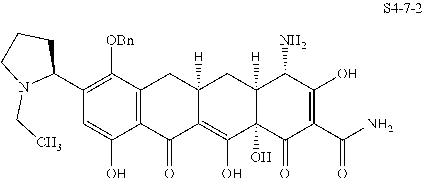 [SMILES: CCN1CCC[C@H]1c1cc(O)c2c(c1OCc1ccccc1)C[C@H]1C[C@H]3[C@H](N)C(O)=C(C(N)=O)C(=O)[C@@]3(O)C(O)=C1C2=O]

S4-7-2: ¹H NMR (400 MHz, CD₃OD, dihydrochloride salt) δ 7.32-7.40 (m, 5H), 6.98 (s, 1H), 4.68-4.72 (m, 2H), 4.47-4.51 (m, 1H), 3.89 (s, 1H), 3.67-3.72 (m, 1H), 2.92-3.11 (m, 4H), 2.61-2.67 (m, 1H), 2.45-2.52 (m, 1H), 2.00-2.25 (m, 5H), 1.75-1.81 (m, 1H), 1.55-1.62 (m, 1H), 1.28 (t, J=5.6 Hz, 3H); MS (ESI) m/z 590.3 (M+H).

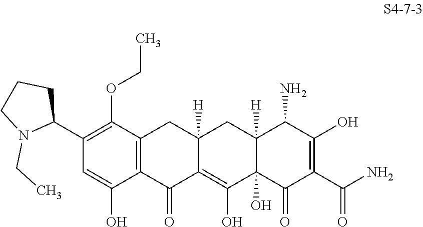 [SMILES: CCOc1c([C@@H]2CCCN2CC)cc(O)c2c1C[C@H]1C[C@H]3[C@H](N)C(O)=C(C(N)=O)C(=O)[C@@]3(O)C(O)=C1C2=O]

S4-7-3: ¹H NMR (400 MHz, CD₃OD, dihydrochloride salt) δ 7.09 (s, 1H), 3.89 (s, 1 H), 3.78-3.88 (m, 2H), 3.68-3.75 (m, 1H), 3.32-3.40 (m, 2H), 3.05-3.22 (m, 3H), 2.90-2.98 (m, 1H), 2.53-2.62 (m, 2H), 2.21-2.40 (m, 5H), 1.55-1.64 (m, 1H), 1.39 (t, J=5.6 Hz, 3H); 1.25 (t, J=5.6 Hz, 3H); MS (ESI) m/z 528.2 (M+H).

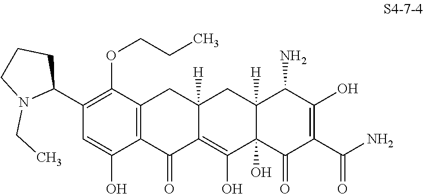 [SMILES: CCCOc1c([C@@H]2CCCN2CC)cc(O)c2c1C[C@H]1C[C@H]3[C@H](N)C(O)=C(C(N)=O)C(=O)[C@@]3(O)C(O)=C1C2=O]

S4-7-4: ¹H NMR (400 MHz, CD₃OD, dihydrochloride salt) δ 7.09 (s, 1H), 3.89 (s, 1 H), 3.79-3.85 (m, 1H), 3.69-3.75 (m, 1H), 3.57-3.63 (m, 1H), 3.32-3.40 (m, 2H), 3.06-3.22 (m, 3H), 2.89-2.96 (m, 1H), 2.55-2.62 (m, 2H), 2.21-2.40 (m, 6H), 1.79-1.86 (m, 1H), 1.55-1.64 (m, 1H), 1.23 (t, J=5.6 Hz, 3H); 1.05 (t, J=5.6 Hz, 3H); MS (ESI) m/z 542.3 (M+H).

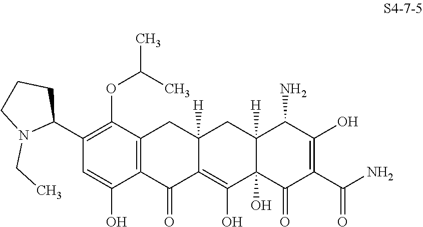 [SMILES: CCN1CCC[C@H]1c1cc(O)c2c(c1OC(C)C)C[C@H]1C[C@H]3[C@H](N)C(O)=C(C(N)=O)C(=O)[C@@]3(O)C(O)=C1C2=O]

S4-7-5: ¹H NMR (400 MHz, CD₃OD, dihydrochloride salt) δ 7.11 (s, 1H), 3.99-4.06 (m, 1H), 3.89 (s, 1H), 3.75-3.82 (m, 1H), 3.32-3.40 (m, 2H), 3.02-3.21 (m, 3H), 2.88-2.94 (m, 1H), 2.53-2.67 (m, 2H), 2.20-2.38 (m, 6H), 1.55-1.65 (m, 1H), 1.36 (d, J=7.6 Hz, 3H), 1.21 (t, J=6.0 Hz, 3H); 1.12 (d, J=7.6 Hz, 3H); MS (ESI) m/z 542.3 (M+H).

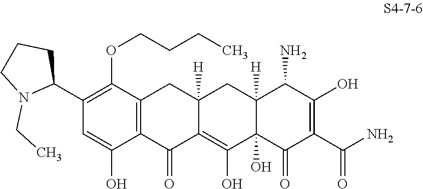 [SMILES: CCCCOc1c([C@@H]2CCCN2CC)cc(O)c2c1C[C@H]1C[C@H]3[C@H](N)C(O)=C(C(N)=O)C(=O)[C@@]3(O)C(O)=C1C2=O]

S4-7-6: ¹H NMR (400 MHz, CD₃OD, dihydrochloride salt) δ 7.09 (s, 1H), 3.89 (s, 1 H), 3.73-3.86 (m, 2H), 3.59-3.65 (m, 1H), 3.32-3.40 (m, 2H), 3.06-3.25 (m, 3H), 2.89-2.96 (m, 1H), 2.55-2.67 (m, 2H), 2.21-2.38 (m, 5H), 1.75-1.83 (m, 2H), 1.48-1.60 (m, 3H), 1.24 (t, J=5.6 Hz, 3H); 0.98 (t, J=5.6 Hz, 3H); MS (ESI) m/z 556.3 (M+H).

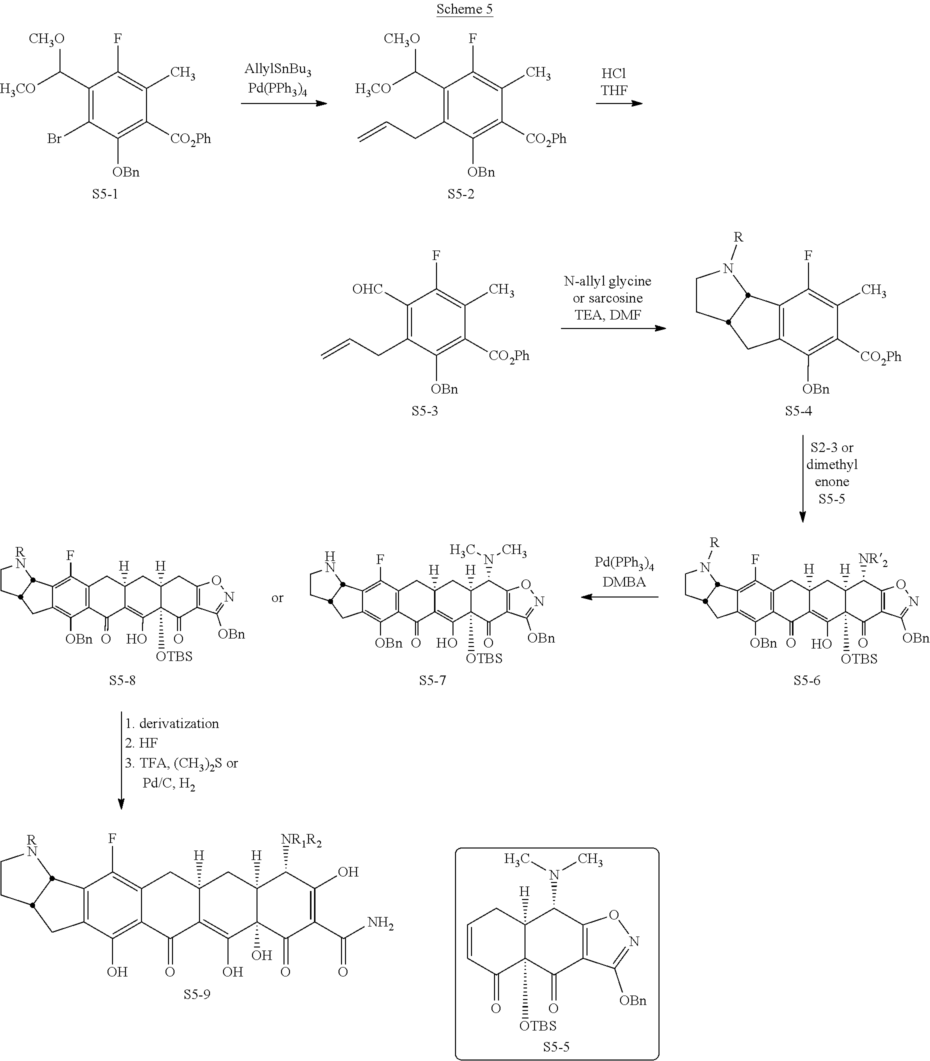

The following compounds were prepared per Scheme 5.

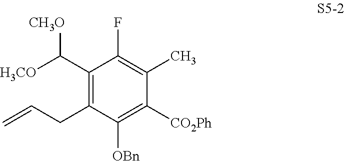

To a solution of compound S5-1 (1.71 g, 3.50 mmol, 1 eq, prepared per literature procedures: J Med. Chem., 2013, 56, 8112-8138) and Pd(PPh₃)₄ (404 mg, 0.35 mmol, 0.1 eq) in toluene (15 mL) was added allyltributyltin (1.29 mL, 4.2 mmol, 1.2 eq) under nitrogen. The resulting reaction mixture was refluxed in a preheated oil bath with a cold water condenser on the top. The reaction turned into a clear solution upon heating. The reaction was heated for 20 h and cooled down to rt. The reaction was concentrated by rotovap. The residue was purified by flash column chromatography (50 g silica gel, 1→10% EtOAc/hexane) to afford the desired product S5-2 (1.55 g, 97%): ¹H NMR (400 MHz, CDCl₃) δ 7.42-7.33 (m, 7H), 7.26-7.24 (m, 1H), 7.05-7.03 (m, 2H), 6.06-6.00 (m, 1H), 5.53 (d, J=3.0 Hz, 1H), 5.06-4.98 (m, 4H), 3.71-3.67 (m, 2H), 3.44 (d, J=3.0 Hz, 6H), 2.35 (s, 3H); MS (ESI) m/z 499.29 (M−H).

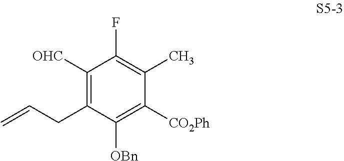

Compound S5-2 (1.55 g, 3.4 mmol, 1 eq) was dissolved in a premixed solution of THF (9.17 mL) and 6 N aq HCl (0.83 mL). The resulting reaction solution was stirred at room temperature for 1 h. Saturated NaHCO₃ and EtOAc were added. The organic phase was separated and concentrated by rotovap. The residue was purified by flash column chromatography (50 g silica gel, 1→10% EtOAc/hexane) to afford the desired product S5-3 as a white solid (1.24 g, 90%): ¹H NMR (400 MHz, CDCl₃) δ 10.46 (s, 1H), 7.41-7.34 (m, 7H), 7.27-7.24 (m, 1H), 7.05-7.03 (m, 2H), 6.05-5.96 (m, 1H), 5.06-5.03 (m, 1H), 4.98 (s, 2H), 4.98-4.91 (m, 1H), 3.87-3.86 (m, 2H), 2.40 (d, J=2.4 Hz, 3H); MS (ESI) m/z 403.27 (M−H).

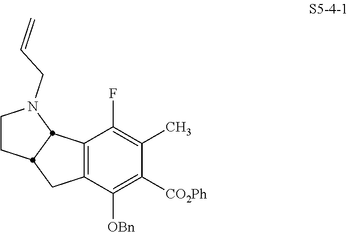

To a mixture of compound S5-3 (702 mg, 1.74 mmol, 1 eq) and N-allylglycine HCl (439 mg, 2.89 mmol, 1.7 eq) was added DMF (8 mL) under nitrogen, followed by TEA (408 μL, 2.89 mmol, 1.7 eq). The resulting reaction mixture was stirred at 80° C. for 1 h 45 min, and cooled to rt. The resulting reaction mixture was then partitioned between EtOAc and water. The organic phase was separated, washed with brine, and concentrated under reduced pressure. Flash chromatography on silica gel using 10%→40% EtOAc/hexanes yielded the desired product S5-4-1 as a white solid (650 mg, 82%): ¹H NMR (400 MHz, CDCl₃) δ 7.41-7.34 (m, 7H), 7.26-7.22 (m, 1H), 7.07-7.04 (m, 2H), 6.01-5.97 (m, 1H), 5.26-5.14 (m, 2H), 5.01 (s, 2H), 4.30 (br s, 1H), 3.79 (br s, 1H), 3.21-3.09 (m, 4H), 2.87 (br d, J=15.9 Hz, 1H), 2.52 (br s, 1H), 2.35 (s, 3H), 2.13 (br s, 1H), 1.66 (br s, 1H); MS (ESI) m/z 458.30 (M+H).

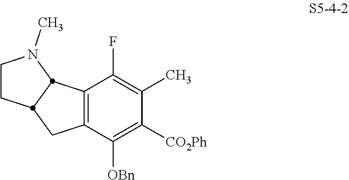

To a mixture of compound S5-3 (290 mg, 0.72 mmol, 1 eq) and sarcosine (76 mg, 0.86 mmol, 1.2 eq) was added DMF (3 mL) under nitrogen. The resulting reaction mixture was stirred at 80° C. for 2 h 30 min, and cooled to rt. The resulting reaction mixture was then partitioned between EtOAc and water. The organic phase was separated, washed with brine, and concentrated under reduced pressure. Flash chromatography on silica gel using 10%→100% EtOAc/hexanes yielded the desired product S5-4-2 as a white solid (250 mg, 81%): ¹H NMR (400 MHz, CDCl₃) δ 7.41-7.34 (m, 7H), 7.26-7.22 (m, 1H), 7.06-7.04 (m, 2 H), 5.04, 5.00 (ABq, J=11.0 Hz, 2H), 4.09 (br s, 1H), 3.24-3.12 (m, 3H), 2.88 (br d, J=12.8 Hz, 1H), 2.64 (s, 3H), 2.56 (br s, 1H), 2.35 (d, J=1.8 Hz, 3H), 2.21-2.12 (m, 1H), 1.76-1.69 (m, 1H); MS (ESI) m/z 432.24 (M+H).

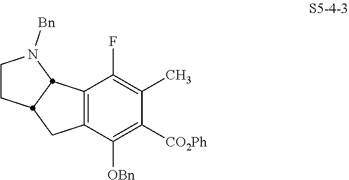

To a mixture of compound S5-3 (575 mg, 1.42 mmol, 1 eq) and N-benzylglycine HCl (344 mg, 1.71 mmol, 1.2 eq) was added DMF (6 mL) under nitrogen, followed by TEA (302 μL, 2.13 mmol, 1.5 eq). The resulting reaction mixture was stirred at 80° C. for 2 h 30 min, and cooled to rt. The resulting reaction mixture was then partitioned between EtOAc and water. The organic phase was separated, washed with brine, and concentrated under reduced pressure. Flash chromatography on silica gel using 1%→20% EtOAc/hexanes yielded the desired product S5-4-3 as a white solid (600 mg, 83%): ¹H NMR (400 MHz, CDCl₃) δ 7.42-7.30 (m, 12H), 7.26-7.22 (m, 1H), 7.08-7.05 (m, 2H), 5.03 (s, 2H), 4.39 (br s, 2H), 3.63-3.61 (m, 1 H), 3.16-3.12 (m, 2H), 2.89-2.86 (m, 2H), 2.44-2.42 (m, 1H), 2.38 (d, J=1.8 Hz, 3H), 2.08 (br s, 1H), 1.60-1.56 (m, 1H); MS (ESI) m/z 508.27 (M+H).

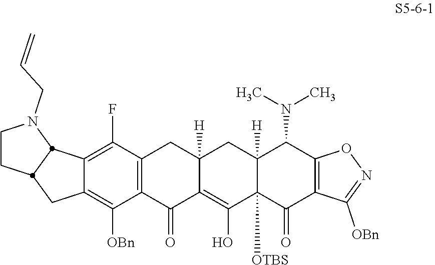 [SMILES: C=CCN1CC[C@H]2Cc3c(OCc4ccccc4)c4c(c(F)c3[C@H]21)C[C@H]1C[C@H]2[C@H](N(C)C)c3onc(OCc5ccccc5)c3C(=O)[C@@]2(O[Si](C)(C)C(C)(C)C)C(O)=C1C4=O]

Compound S5-6-1 was prepared from S5-4-1 (650 mg, 1.42 mmol, 1 eq) and C-4 dimethylamino enone S5-5 (690 mg, 1.42 mmol, 1 eq) by using General Procedure E. Product S5-6-1 (957 mg, a mixture of diastereomers, 80%): ¹H NMR (400 MHz, CDCl₃) δ 16.08 (s, 0.5 H), 16.05 (s, 0.5 H), 7.50-7.48 (m, 2H), 7.41-7.30 (m, 8H), 6.02-5.94 (m, 1H), 5.36 (s, 2 H), 5.22 (br d, J=16.5 Hz, 1H), 5.14 (br d, J=9.2 Hz, 1H), 4.93-4.85 (m, 2H), 4.33-4.26 (m, 1H), 3.98-3.94 (m, 1H), 3.84-3.76 (m, 1H), 3.26-3.22 (m, 2H), 3.06-2.92 (m 4 H), 2.80-2.65 (m, 1H), 2.56-2.41 (m, 9H), 2.14-2.10 (m, 2H), 1.70-1.49 (m, 1H), 0.82 (s, 4.5 H), 0.81 (s, 4.5 H), 0.27 (s, 3H), 0.12 (s, 3H); MS (ESI) m/z 846.62 (M+H).

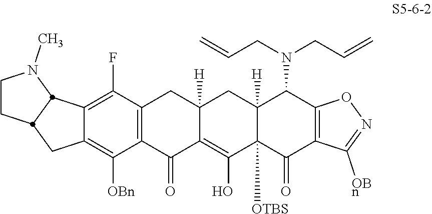 [SMILES: C=CCN(CC=C)[C@@H]1c2onc(OCc3ccccc3)c2C(=O)[C@@]2(O[Si](C)(C)C(C)(C)C)C(O)=C3C(=O)c4c(c(F)c5c(c4OCc4ccccc4)C[C@@H]4CCN(C)[C@H]54)C[C@H]3C[C@@H]12]

Compound S5-6-2 was prepared from S5-4-2 (250 mg, 0.58 mmol, 1 eq) and C-4 diallylamino enone S2-3 (310 mg, 0.58 mmol, 1 eq) by using General Procedure E. Product S5-6-2 (421 mg, a mixture of diastereomers, 83%): ¹H NMR (400 MHz, CDCl₃) δ 15.84 (br s, 1H), 7.41-7.39 (m, 2H), 7.29-7.23 (m, 8H), 5.75-5.65 (m, 1H), 5.26 (s, 2H), 5.13-5.09 (m, 2H), 5.02-5.00 (m, 2H), 4.82-4.68 (m, 2H), 3.97-3.95 (m, 1H), 3.24-2.88 (m, 10H), 2.55-2.34 (m, 7H), 2.09-2.01 (m, 2H), 0.71 (s, 4.5 H), 0.69 (s, 4.5 H), 0.16 (s, 1.5 H), 0.15 (s, 1.5 H), 0.00 (s, 3H); MS (ESI) m/z 872.56 (M+H).

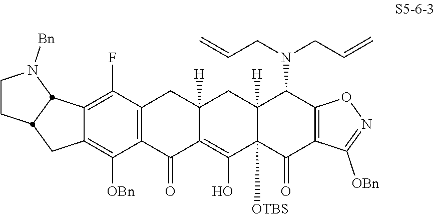 [SMILES: C=CCN(CC=C)[C@@H]1c2onc(OCc3ccccc3)c2C(=O)[C@@]2(O[Si](C)(C)C(C)(C)C)C(O)=C3C(=O)c4c(c(F)c5c(c4OCc4ccccc4)C[C@@H]4CCN(Cc6ccccc6)[C@H]54)C[C@H]3C[C@@H]12]

Compound S5-6-3 was prepared from S5-4-3 (600 mg, 1.18 mmol, 1 eq) and C-4 diallylamino enone S2-3 (631 mg, 1.18 mmol, 1 eq) by using General Procedure E. Diastereomer B (S5-6-3B, 405 mg, 36%) of product S5-6-3 was isolated by flash column chromatography. But diastereomer A (S5-6-3A, 570 mg, 51%) was mixed with a small amount of diastereomer. S5-6-3A: ¹H NMR (400 MHz, CDCl₃) δ 16.03 (s, 1H), 7.53-7.51 (m, 2H), 7.51-7.31 (m, 12H), 7.28-7.24 (m, 1H), 5.88-5.78 (m, 2H), 5.39 (s, 2H), 5.24 (d, J=17.1 Hz, 2H), 5.14 (d, J=9.8 Hz, 2H), 4.89-4.82 (m, 2H), 4.46-4.40 (m, 2H), 4.11 (d, J=10.4 Hz, 1H), 3.67 (d, J=12.8 Hz, 1H), 3.36-3.33 (m, 2H), 3.27-3.21 (m, 3H), 3.10-3.02 (m, 3 H), 2.85-2.83 (m, 1H), 2.72-2.43 (m, 4H), 2.16 (d, J=14.0 Hz, 1H), 2.05-2.02 (m, 1H), 1.58-1.45 (m, 2H), 0.85 (s, 9H), 0.28 (s, 3H), 0.14 (s, 3H). S5-6-3B: ¹H NMR (400 MHz, CDCl₃) δ 16.03 (s, 1H), 7.53-7.51 (m, 2H), 7.43-7.30 (m, 12H), 7.26-7.24 (m, 1H), 5.88-5.78 (m, 2H), 5.39 (s, 2H), 5.24 (d, J=17.1 Hz, 2H), 5.17 (d, J=9.8 Hz, 2H), 4.91, 4.87 (ABq, J=11.0 Hz, 2H), 4.13 (d, J=9.8 Hz, 1H), 3.68 (br d, J=12.2 Hz, 1H), 3.39-3.19 (m, 5H), 3.02-2.78 (m, 4H), 2.67-2.63 (m, 1H), 2.58-2.54 (m, 1H), 2.51-2.43 (m, 2H), 2.17 (br d, J=14.6 Hz, 1H), 2.10-2.05 (m, 1H), 1.58-1.55 (m, 2H), 0.83 (s, 9H), 0.28 (s, 3H), 0.13 (s, 3H); MS (ESI) m/z 948.56 (M+H).

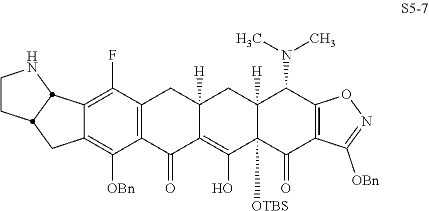 [SMILES: CN(C)[C@@H]1c2onc(OCc3ccccc3)c2C(=O)[C@@]2(O[Si](C)(C)C(C)(C)C)C(O)=C3C(=O)c4c(c(F)c5c(c4OCc4ccccc4)C[C@@H]4CCN[C@H]54)C[C@H]3C[C@@H]12]

Compound S5-7 was prepared from S5-6-1 (205 mg, 0.24 mmol, 1 eq) by using General Procedure A (168 mg, a mixture of diastereomers, 86%): ¹H NMR (400 MHz, CDCl₃) δ 7.66-7.61 (m, 1H), 7.53-7.44 (m, 3H), 7.38-7.32 (m, 6H), 5.36 (s, 2H), 4.98-4.82 (m, 3H), 3.95 (d, J=10.4 Hz, 1H), 3.25-3.22 (m, 1H), 3.14-3.00 (m, 4H), 2.77-2.65 (m, 2H), 2.56-2.37 (m, 9H), 2.13 (br d, J=14.6 Hz, 1H), 1.98-1.95 (m, 1H), 1.56-1.44 (m, 1H), 0.82 (s, 4.5 H), 0.81 (s, 4.5 H), 0.27 (s, 3H), 0.12 (s, 3H); MS (ESI) m/z 806.55 (M+H).

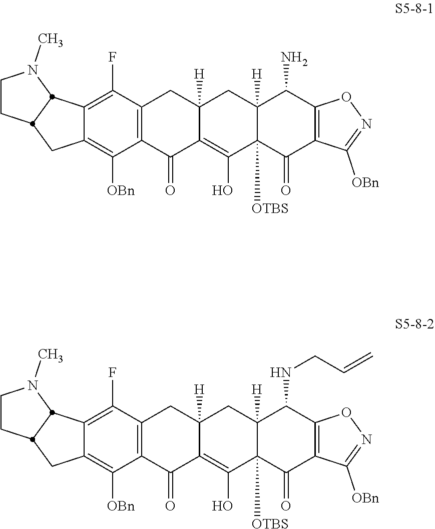 [SMILES: CN1CC[C@H]2Cc3c(OCc4ccccc4)c4c(c(F)c3[C@H]21)C[C@H]1C[C@H]2[C@H](N)c3onc(OCc5ccccc5)c3C(=O)[C@@]2(O[Si](C)(C)C(C)(C)C)C(O)=C1C4=O ; C=CCN[C@@H]1c2onc(OCc3ccccc3)c2C(=O)[C@@]2(O[Si](C)(C)C(C)(C)C)C(O)=C3C(=O)c4c(c(F)c5c(c4OCc4ccccc4)C[C@@H]4CCN(C)[C@H]54)C[C@H]3C[C@@H]12]

Compounds S5-8-1 and S5-8-2 were prepared from S5-6-2 (377 mg, 0.43 mmol, 1 eq) by using General Procedure A. S5-8-1 (198 mg, a mixture of diastereomers, 58%): MS (ESI) m/z 792.46 (M+H). S5-8-2 (58 mg, a mixture of diastereomers, 16%): MS (ESI) m/z 832.49 (M+H).

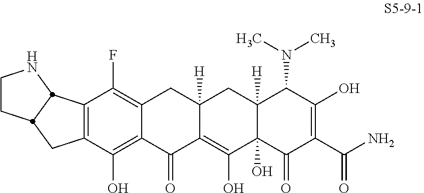 [SMILES: CN(C)[C@@H]1C(O)=C(C(N)=O)C(=O)[C@@]2(O)C(O)=C3C(=O)c4c(O)c5c(c(F)c4C[C@H]3C[C@@H]12)[C@H]1NCC[C@H]1C5]

Compound S5-9-1 was prepared from S5-7 (42 mg, 0.052 mmol, 1 eq) by using General Procedures C and D-1. The two diastereomers of S5-9-1 were separated by preparative reverse phase HPLC. S5-9-1A: ¹H NMR (400 MHz, CD₃OD, dihydrochloride salt) δ 5.36 (d, J=8.8 Hz, 1H), 4.11 (s, 1H), 3.50-3.45 (m, 1H), 3.36-3.33 (m, 2H), 3.27-3.18 (m, 2H), 3.12-2.89 (m, 9H), 2.50-2.42 (m, 1H), 2.34-2.22 (m, 2H), 1.86-1.77 (m, 1H), 1.68-1.58 (m, 1H). S5-9-1B: ¹H NMR (400 MHz, CD₃OD, dihydrochloride salt) δ 5.36 (d, J=8.8 Hz, 1H), 4.11 (s, 1H), 3.51-3.43 (m, 1H), 3.37-3.33 (m, 2H), 3.27-3.17 (m, 2H), 3.12-2.87 (m, 9H), 2.50-2.42 (m, 1H), 2.34-2.22 (m, 2H), 1.86-1.77 (m, 1H), 1.68-1.58 (m, 1H); MS (ESI) m/z 514.32 (M+H).

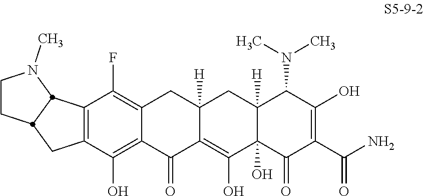 [SMILES: CN(C)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compound S5-9-2 was prepared from S5-7 (21 mg, 0.026 mmol, 1 eq) and HCHO by using General Procedures B-1, C and D-1. The two diastereomers of S5-9-2 were separated by preparative reverse phase HPLC. S5-9-2A: ¹H NMR (400 MHz, CD₃OD, dihydrochloride salt) 55.22 (d, J=8.8 Hz, 1H), 4.11 (s, 1H), 3.72-3.68 (m, 1H), 3.61-3.57 (m, 1H), 3.36-3.30 (m, 1H), 3.24-3.18 (m, 5H), 3.13-3.05 (m, 4H), 3.00-2.92 (m, 5H), 2.60-2.56 (m, 1H), 2.37-2.24 (m, 2H), 1.85-1.75 (m, 1H), 1.69-1.60 (m, 1H). S5-9-2B: ¹H NMR (400 MHz, CD₃OD, dihydrochloride salt) δ 5.21 (d, J=8.4 Hz, 1H), 4.11 (s, 1H), 3.72-3.68 (m, 1H), 3.61-3.57 (m, 1H), 3.35-3.30 (m, 1H), 3.26-3.20 (m, 5H), 3.13-3.05 (m, 4H), 3.01-2.89 (m, 5H), 2.62-2.55 (m, 1H), 2.36-2.23 (m, 2H), 1.85-1.79 (m, 1H), 1.69-1.59 (m, 1H); MS (ESI) m/z 528.27 (M+H).

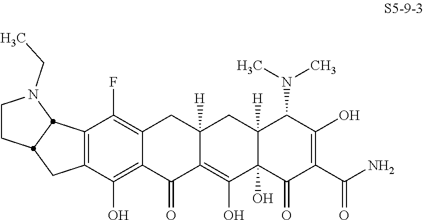 [SMILES: CCN1CC[C@H]2Cc3c(O)c4c(c(F)c3[C@H]21)C[C@H]1C[C@H]2[C@H](N(C)C)C(O)=C(C(N)=O)C(=O)[C@@]2(O)C(O)=C1C4=O]

Compound S5-9-3 was prepared from S5-7 (42 mg, 0.052 mmol, 1 eq) and CH₃CHO by using General Procedures B-1, C and D-1. The two diastereomers of S5-9-3 were separated by preparative reverse phase HPLC. S5-9-3A: ¹H NMR (400 MHz, CD₃OD, dihydrochloride salt) δ 5.29 (d, J=8.8 Hz, 1H), 4.13 (s, 1H), 3.86-3.77 (m, 1H), 3.74-3.69 (m, 1H), 3.58-3.53 (m, 1H), 3.42-3.37 (m, 1H), 3.28-2.92 (m, 12H), 2.60-2.52 (m, 1H), 2.36-2.25 (m, 2 H), 1.85-1.75 (m, 1H), 1.69-1.59 (m, 1H), 1.42 (t, J=7.2 Hz, 3H). S5-9-3B: ¹H NMR (400 MHz, CD₃OD, dihydrochloride salt) δ 5.27 (d, J=8.8 Hz, 1H), 4.12 (s, 1H), 3.85-3.78 (m, 1 H), 3.75-3.70 (m, 1H), 3.57-3.54 (m, 1H), 3.42-3.37 (m, 1H), 3.28-3.19 (m, 2H), 3.14-2.90 (m, 10H), 2.60-2.52 (m, 1H), 2.34-2.25 (m, 2H), 1.86-1.76 (m, 1H), 1.68-1.59 (m, 1H), 1.44 (t, J=7.6 Hz, 3H); MS (ESI) m/z 542.37 (M+H).

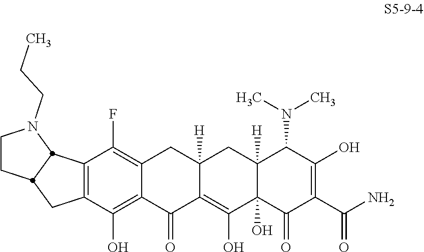 [SMILES: CCCN1CC[C@H]2Cc3c(O)c4c(c(F)c3[C@H]21)C[C@H]1C[C@H]2[C@H](N(C)C)C(O)=C(C(N)=O)C(=O)[C@@]2(O)C(O)=C1C4=O]

Compound S5-9-4 was prepared from S5-6-1 (46 mg, 0.054 mmol, 1 eq) by using General Procedures C and D-2. The two diastereomers of S5-9-4 were separated by preparative reverse phase HPLC. S5-9-4A: ¹H NMR (400 MHz, CD₃OD, dihydrochloride salt) δ 5.30 (d, J=8.4 Hz, 1H), 4.13 (s, 1H), 3.74-3.62 (m, 2H), 3.57-3.53 (m, 1H), 3.39-3.29 (m, 1H), 3.24-3.17 (m, 2H), 3.12-2.92 (m, 10H), 2.60-2.53 (m, 1H), 2.36-2.25 (m, 2H), 1.88-1.76 (m, 3H), 1.69-1.59 (m, 1H), 1.06 (t, J=7.2 Hz, 3H). S5-9-4B: ¹H NMR (400 MHz, CD₃OD, dihydrochloride salt) δ 5.28 (d, J=8.4 Hz, 1H), 4.11 (s, 1H), 3.74-3.61 (m, 2H), 3.56-3.53 (m, 1H), 3.34-3.29 (m, 1H), 3.27-3.18 (m, 2H), 3.12-2.92 (m, 10H), 2.59-2.53 (m, 1H), 2.32-2.25 (m, 2H), 1.88-1.76 (m, 3H), 1.67-1.57 (m, 1H), 1.06 (t, J=7.6 Hz, 3H); MS (ESI) m/z 556.33 (M+H).

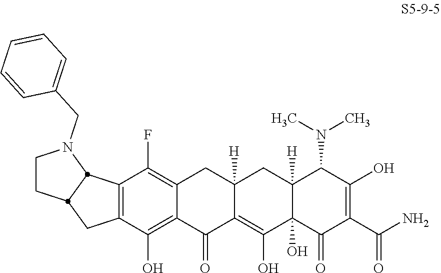 [SMILES: CN(C)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1Cc1ccccc1)C5]

Compound S5-9-5 was prepared from S5-7 (42 mg, 0.052 mmol, 1 eq) and PhCHO by using General Procedures B-1, C and D-1. The two diastereomers of S5-9-5 were separated by preparative reverse phase HPLC. S5-9-5A: ¹H NMR (400 MHz, CD₃OD, dihydrochloride salt) δ 7.56-7.53 (m, 2H), 7.50-7.49 (m, 3H), 5.44 (d, J=8.8 Hz, 1H), 4.94 (d, J=13.2 Hz, 1H), 4.48 (d, J=13.2 Hz, 1H), 4.10 (s, 1H), 3.61-3.57 (m, 1H), 3.44-3.42 (m, 1H), 3.34-3.30 (m, 2H), 3.28-2.91 (m, 10H), 2.58-2.52 (m, 1H), 2.40-2.23 (m, 2H), 1.76-1.64 (m, 2H). S5-9-5B: ¹H NMR (400 MHz, CD₃OD, dihydrochloride salt) δ 7.58-7.56 (m, 2H), 7.51-7.49 (m, 3 H), 5.43 (d, J=8.8 Hz, 1H), 4.94 (d, J=13.2 Hz, 1H), 4.51 (d, J=13.2 Hz, 1H), 4.13 (s, 1 H), 3.62-3.58 (m, 1H), 3.47-3.41 (m, 1H), 3.34-3.20 (m, 3H), 3.15-2.91 (m, 9H), 2.58-2.52 (m, 1H), 2.37-2.26 (m, 2H), 1.78-1.64 (m, 2H); MS (ESI) m/z 604.41 (M+H).

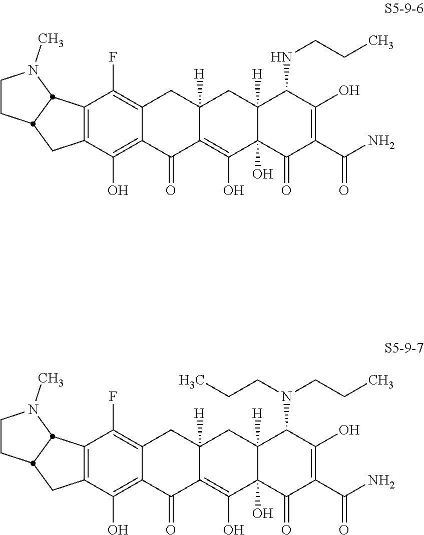 [SMILES: CCCN[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5 ; CCCN(CCC)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compounds S5-9-6 and S5-9-7 were prepared from S5-6-2 (44 mg, 0.050 mmol, 1 eq) by using General Procedures C and D-2. The two diastereomers of S5-9-6 were separated by preparative reverse phase HPLC, while S5-9-7 was isolated as a mixture of diastereomers. S5-9-6A: ¹H NMR (400 MHz, CD₃OD, dihydrochloride salt) δ 5.21 (d, J=9.2 Hz, 1H), 3.88 (s, 1H), 3.70-3.66 (m, 1H), 3.60-3.57 (m, 1H), 3.34-3.29 (m, 2H), 3.26-3.16 (m, 6H), 3.04-2.98 (m, 1H), 2.94-2.85 (m, 2H), 2.61-2.54 (m, 1H), 2.35-2.22 (m, 2H), 1.83-1.72 (m, 3H), 1.61-1.51 (m, 1H), 1.02 (t, J=7.1 Hz, 3H). S5-9-6B: ¹H NMR (400 MHz, CD₃OD, dihydrochloride salt) δ 5.21 (d, J=8.7 Hz, 1H), 3.89 (s, 1H), 3.72-3.68 (m, 1H), 3.61-3.57 (m, 1H), 3.55-3.29 (m, 2H), 3.26-3.19 (m, 6H), 3.06-2.98 (m, 1H), 2.93-2.87 (m, 2H), 2.61-2.55 (m, 1H), 2.34-2.22 (m, 2H), 1.85-1.73 (m, 3H), 1.61-1.52 (m, 1H), 1.03 (t, J=7.3 Hz, 3H); MS (ESI) m/z 542.30 (M+H).

S5-9-7: ¹H NMR (400 MHz, CD₃OD, dihydrochloride salt, a mixture of diastereomers) 55.23-5.20 (m, 1H), 4.23 (s, 1H), 3.73-3.68 (m, 1H), 3.61-3.57 (m, 1H), 3.51-3.47 (m, 1H), 3.38-3.33 (m, 2H), 3.26-3.20 (m, 7H), 3.10-3.04 (m, 1H), 2.99-2.89 (m, 3H), 2.36-2.22 (m, 2H), 1.86-1.76 (m, 5H), 1.69-1.59 (m, 1H), 1.05-0.98 (m, 6H); MS (ESI) m/z 584.3 (M+H).

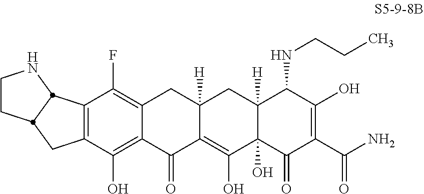 [SMILES: CCCN[C@@H]1C(O)=C(C(N)=O)C(=O)[C@@]2(O)C(O)=C3C(=O)c4c(O)c5c(c(F)c4C[C@H]3C[C@@H]12)[C@H]1NCC[C@H]1C5]

Compounds S5-9-8B was prepared from S5-6-3B (20 mg, 0.021 mmol, 1 eq) by using General Procedures C and D-2: ¹H NMR (400 MHz, CD₃OD, dihydrochloride salt) δ 5.34 (d, J=8.8 Hz, 1H), 3.88 (s, 1H), 3.48-3.43 (m, 1H), 3.35-3.32 (m, 3H), 3.26-3.16 (m, 3H), 3.05-2.96 (m, 1H), 2.93-2.85 (m, 2H), 2.49-2.41 (m, 1H), 2.32-2.21 (m, 2H), 1.85-1.72 (m, 3H), 1.60-1.51 (m, 1H), 1.02 (t, J=7.2 Hz, 3H); MS (ESI) m/z 528.29 (M+H).

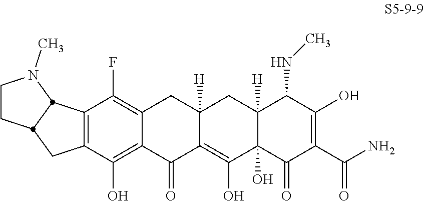 [SMILES: CN[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compound S5-9-9 was prepared from S5-8-2 (58 mg, 0.07 mmol, 1 eq) and HCHO by using General Procedures B-1 and A. Half of the material was processed per General Procedures C and D-1 to give product S5-9-9. The two diastereomers of S5-9-9 were separated by preparative reverse phase HPLC. S5-9-9A: ¹H NMR (400 MHz, CD₃OD, dihydrochloride salt) δ 5.21 (d, J=8.8 Hz, 1H), 3.82 (s, 1H), 3.71-3.67 (m, 1H), 3.63-3.56 (m, 1H), 3.35-3.31 (m, 1H), 3.23-3.16 (m, 5H), 3.06-2.91 (m, 5H), 2.83-2.80 (m, 1H), 2.61-2.55 (m, 1H), 2.35-2.28 (m, 1H), 2.25-2.21 (m, 1H), 1.84-1.74 (m, 1H), 1.62-1.52 (m, 1H). S5-9-9B: ¹H NMR (400 MHz, CD₃OD, dihydrochloride salt) δ 5.20 (d, J=8.8 Hz, 1H), 3.81 (s, 1H), 3.72-3.68 (m, 1H), 3.61-3.56 (m, 1H), 3.35-3.30 (m, 1H), 3.26-3.18 (m, 5H), 3.06-2.97 (m, 1H), 2.95-2.89 (m, 4H), 2.83-2.76 (m, 1H), 2.62-2.55 (m, 1H), 2.36-2.28 (m, 1H), 2.25-2.20 (m, 1H), 1.85-1.75 (m, 1H), 1.63-1.53 (m, 1H); MS (ESI) m/z 514.27 (M+H).

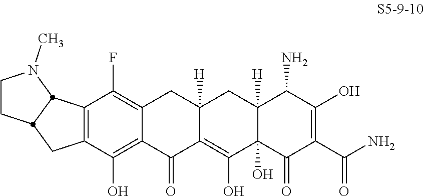 [SMILES: CN1CC[C@H]2Cc3c(O)c4c(c(F)c3[C@H]21)C[C@H]1C[C@H]2[C@H](N)C(O)=C(C(N)=O)C(=O)[C@@]2(O)C(O)=C1C4=O]

Compound S5-9-10 was prepared from S5-8-1 (30 mg, 0.38 mmol, 1 eq) by using General Procedures C and D-1 to give product S5-9-10. The two diastereomers of S5-9-10 were separated by preparative reverse phase HPLC. S5-9-10A: ¹H NMR (400 MHz, CD₃OD, dihydrochloride salt) S5.22 (d, J=8.8 Hz, 1H), 3.89 (s, 1H), 3.72-3.67 (m, 1H), 3.62-3.57 (m, 1H), 3.35-3.28 (m, 1H), 3.23-3.17 (m, 5H), 3.04-2.91 (m, 2H), 2.72-2.65 (m, 1H), 2.62-2.55 (m, 1H), 2.37-2.30 (m, 1H), 2.28-2.23 (m, 1H), 1.84-1.77 (m, 1H), 1.64-1.54 (m, 1H). S5-9-10B: ¹H NMR (400 MHz, CD₃OD, dihydrochloride salt) δ 5.21 (d, J=9.2 Hz, 1H), 3.90 (s, 1H), 3.72-3.68 (m, 1H), 3.62-3.57 (m, 1H), 3.35-3.29 (m, 1H), 3.25-3.19 (m, 5H), 3.04-2.96 (m, 1H), 2.93-2.87 (m, 1H), 2.69-2.65 (m, 1H), 2.62-2.55 (m, 1H), 2.36-2.23 (m, 2H), 1.86-1.76 (m, 1H), 1.64-1.54 (m, 1H); MS (ESI) m/z 500.26 (M+H).

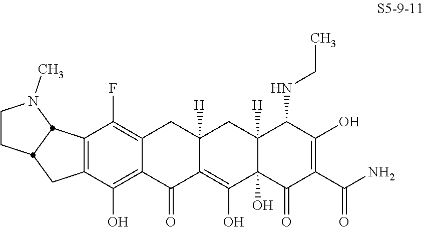 [SMILES: CCN[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compound S5-9-11 was prepared from S5-8-1 and CH₃CHO by using General Procedures B-1, C and D-1 to give product S5-9-11. The two diastereomers of S5-9-11 were separated by preparative reverse phase HPLC. S5-9-11A: ¹H NMR (400 MHz, CD₃OD, dihydrochloride salt) δ 5.22 (d, J=8.4 Hz, 1H), 3.88 (s, 1H), 3.71-3.68 (m, 1H), 3.62-3.57 (m, 1H), 3.46-3.39 (m, 1H), 3.38-3.28 (m, 2H), 3.23-3.17 (m, 5H), 3.05-2.99 (m, 1H), 2.96-2.91 (m, 1H), 2.87-2.83 (m, 1H), 2.62-2.55 (m, 1H), 2.36-2.23 (m, 2H), 1.84-1.74 (m, 1H), 1.62-1.52 (m, 1H), 1.36 (t, J=7.2 Hz, 3H). S5-9-11B: ¹H NMR (400 MHz, CD₃OD, dihydrochloride salt) S5.21 (d, J=8.4 Hz, 1H), 3.88 (s, 1H), 3.72-3.68 (m, 1H), 3.64-3.55 (m, 1H), 3.48-3.41 (m, 1H), 3.38-3.28 (m, 2H), 3.26-3.18 (m, 5H), 3.07-2.99 (m, 1H), 2.96-2.84 (m, 2H), 2.62-2.55 (m, 1H), 2.36-2.22 (m, 2H), 1.84-1.74 (m, 1H), 1.66-1.52 (m, 1H), 1.36 (t, J=7.2 Hz, 3H); MS (ESI) m/z 528.23 (M+H).

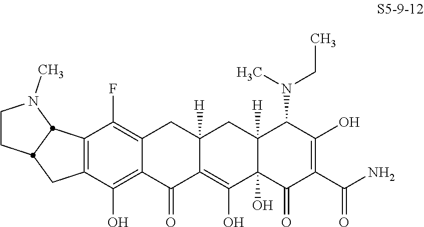 [SMILES: CCN(C)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compound S5-9-12 was prepared from S5-8-1 and CH₃CHO by using General Procedures B-1, and B-1 again with HCHO followed by General Procedures C and D-1 to give product S5-9-12. The two diastereomers of S5-9-12 were separated by preparative reverse phase HPLC. S5-9-12A: ¹H NMR (400 MHz, CD₃OD, dihydrochloride salt) δ 5.22 (d, J=8.8 Hz, 1H), 4.23 (s, 0.5 H), 4.14 (s, 0.5 H), 3.71-3.67 (m, 1H), 3.61-3.56 (m, 1H), 3.50-3.46 (m, 1H), 3.35-3.30 (m, 2H), 3.24-3.17 (m, 5H), 3.10-3.02 (m, 2.5 H), 2.95-2.91 (m, 3.5 H), 2.62-2.55 (m, 1H), 2.36-2.22 (m, 2H), 1.84-1.74 (m, 1H), 1.67-1.58 (m, 1H), 1.43-1.39 (m, 3H). S5-9-12B: ¹H NMR (400 MHz, CD₃OD, dihydrochloride salt) δ 5.21 (d, J=8.8 Hz, 1H), 4.23 (s, 0.5 H), 4.14 (s, 0.5 H), 3.73-3.68 (m, 1H), 3.62-3.57 (m, 1H), 3.52-3.47 (m, 1H), 3.38-3.30 (m, 2H), 3.26-3.20 (m, 5H), 3.09-2.88 (m, 6H), 2.61-2.57 (m, 1H), 2.36-2.22 (m, 2H), 1.85-1.75 (m, 1H), 1.67-1.58 (m, 1H), 1.44-1.39 (m, 3H); MS (ESI) m/z 542.30 (M+H).

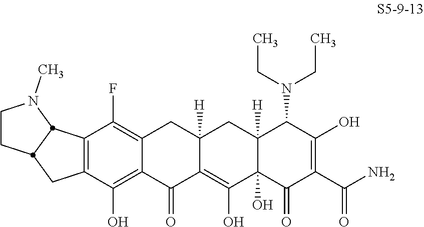 [SMILES: CCN(CC)[C@@H]1C(O)=C(C(N)=O)C(=O)[C@@]2(O)C(O)=C3C(=O)c4c(O)c5c(c(F)c4C[C@H]3C[C@@H]12)[C@@H]1[C@@H](CCN1C)C5]

Compound S5-9-13 was prepared from S5-8-1 and CH₃CHO by using General Procedures B-1, C and D-1 to give product S5-9-13. The two diastereomers of S5-9-13 were separated by preparative reverse phase HPLC. S5-9-13A: ¹H NMR (400 MHz, CD₃OD, dihydrochloride salt) δ 5.22 (d, J=9.2 Hz, 1H), 4.25 (s, 1H), 3.72-3.67 (m, 1H), 3.62-3.54 (m, 2H), 3.48-3.43 (m, 2H), 3.35-3.28 (m, 2H), 3.25-3.17 (m, 5H), 3.09-3.02 (m, 1H), 2.94-2.90 (m, 1H), 2.62-2.54 (m, 1H), 2.36-2.26 (m, 2H), 1.84-1.75 (m, 1H), 1.69-1.59 (m, 1H), 1.41 (t, J=7.2 Hz, 6H). S5-9-13B: ¹H NMR (400 MHz, CD₃OD, dihydrochloride salt) δ 5.21 (d, J=9.2 Hz, 1H), 4.25 (s, 1H), 3.73-3.68 (m, 1H), 3.63-3.55 (m, 2H), 3.50-3.42 (m, 2H), 3.36-3.28 (m, 2H), 3.25-3.19 (m, 5H), 3.12-3.02 (m, 1H), 2.95-2.89 (m, 1H), 2.62-2.54 (m, 1H), 2.34-2.23 (m, 2H), 1.85-1.75 (m, 1H), 1.68-1.59 (m, 1H), 1.41 (t, J=7.2 Hz, 6H); MS (ESI) m/z 556.29 (M+H).

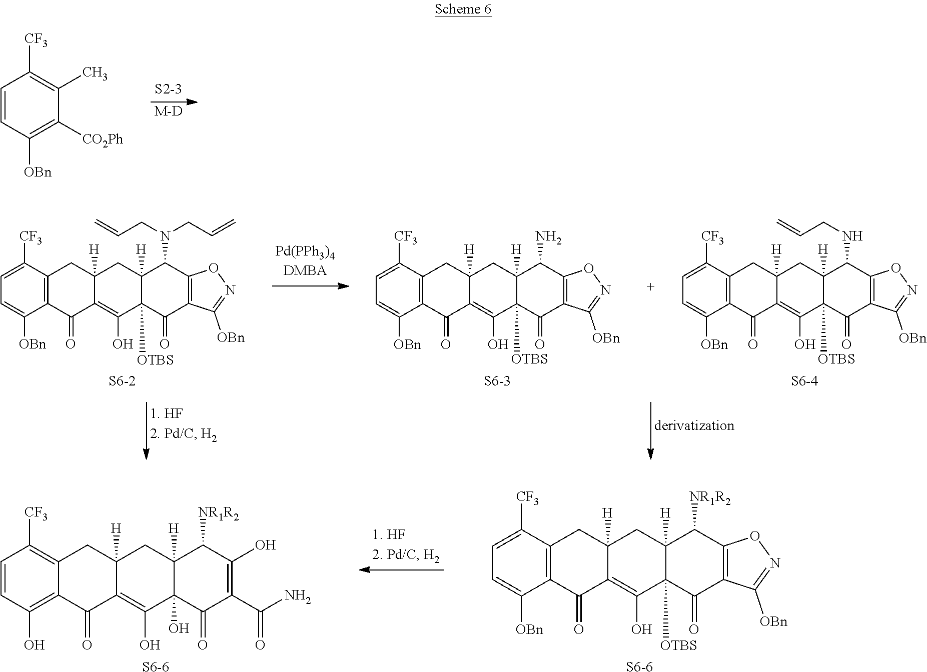

The following compounds were prepared per Scheme 6.

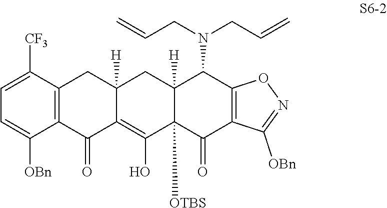 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)ccc(C(F)(F)F)c4C[C@H]3C[C@@H]12]

Compound S6-2 was prepared from compound S6-1 (prepared per literature procedures including WO2011/025982 A2) and diallylenone S2-3 by using General Procedure E:

¹H NMR (400 MHz, CDCl₃) δ 15.91 (s, 1H), 7.65 (d, J=9.2 Hz, 1H), 7.51-7.44 (m, 4H), 7.40-7.27 (m, 6H), 6.93 (d, J=9.2 Hz, 1H), 5.85-5.75 (m, 2H), 5.36 (s, 2H), 5.30-5.19 (m, 4H), 5.11 (d, J=10.0 Hz, 2H), 4.09 (d, J=10.4 Hz, 1H), 3.35-3.32 (m, 2H), 3.22-3.12 (m, 3H), 2.96-2.92 (m, 2H), 2.52-2.45 (m, 2H), 2.14-2.10 (m, 1H), 0.82 (s, 9H), 0.28 (s, 3H), 0.14 (s, 3H); MS (ESI) m/z 827.60 (M+H).

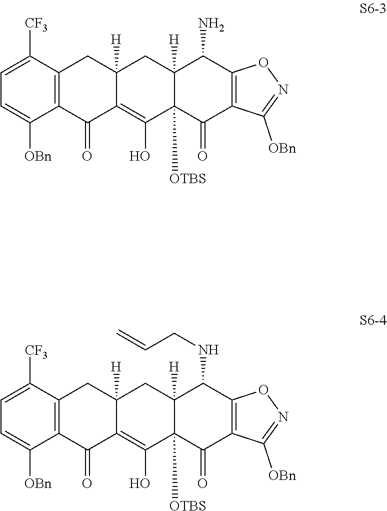 [SMILES: CC(C)(C)[Si](C)(C)O[C@@]12C(=O)c3c(OCc4ccccc4)noc3[C@@H](N)[C@@H]1C[C@@H]1Cc3c(C(F)(F)F)ccc(OCc4ccccc4)c3C(=O)C1=C2O ; C=CCN[C@@H]1c2onc(OCc3ccccc3)c2C(=O)[C@@]2(O[Si](C)(C)C(C)(C)C)C(O)=C3C(=O)c4c(OCc5ccccc5)ccc(C(F)(F)F)c4C[C@H]3C[C@@H]12]

Compounds S6-3 and S6-4 were prepared from compound S6-2 by using General Procedure A. S6-3: ¹H NMR (400 MHz, CDCl₃) δ 16.41 (s, 1H), 7.64 (d, J=9.2 Hz, 1H), 7.52-7.46 (m, 4H), 7.42-7.30 (m, 6H), 6.95 (d, J=9.2 Hz, 1H), 5.45, 5.35 (ABq, J=12.0 Hz, 2H), 5.31, 5.24 (ABq, J=12.8 Hz, 2H), 4.00 (br s, 1H), 3.07-3.03 (m, 1H), 2.88-2.79 (m, 1H), 2.69-2.66 (m, 1H), 2.42 (t, J=15.2 Hz, 1H), 2.17-2.12 (m, 1H), 1.47-1.38 (m, 1 H), 0.74 (s, 9H), 0.23 (s, 3H), 0.10 (s, 3H); MS (ESI) m/z 747.50 (M+H). S6-4: MS (ESI) m/z 787.55 (M+H).

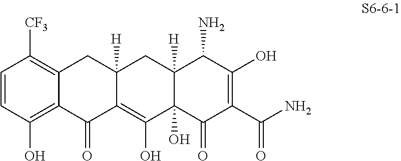 [SMILES: NC(=O)C1=C(O)[C@@H](N)[C@@H]2C[C@@H]3Cc4c(C(F)(F)F)ccc(O)c4C(=O)C3=C(O)[C@]2(O)C1=O]

Compound S6-6-1 was prepared from compound S6-3 by using General Procedures C and D-2: ¹H NMR (400 MHz, CD₃OD, hydrochloride salt) δ 7.75 (d, J=9.2 Hz, 1H), 6.95 (d, J=9.2 Hz, 1H), 3.90 (br s, 1H), 3.22-3.17 (m, 1H), 3.04-2.96 (m, 1H), 2.63 (dt, J=12.4, 2.0 Hz, 1H), 2.54 (t, J=14.8 Hz, 1H), 2.22 (ddd, J=13.2, 4.8, 2.0 Hz, 1H), 1.63-1.54 (m, 1 H); MS (ESI) m/z 455.30 (M+H).

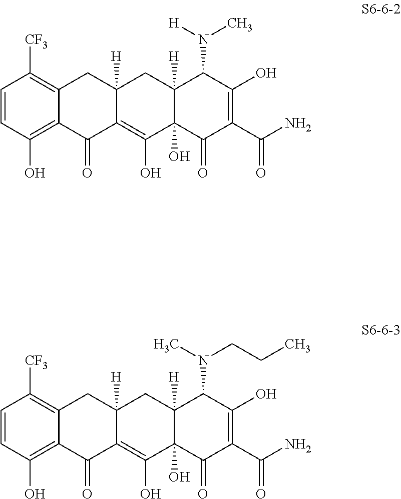 [SMILES: CN[C@@H]1C(O)=C(C(N)=O)C(=O)[C@@]2(O)C(O)=C3C(=O)c4c(O)ccc(C(F)(F)F)c4C[C@H]3C[C@@H]12 ; CCCN(C)[C@@H]1C(O)=C(C(N)=O)C(=O)[C@@]2(O)C(O)=C3C(=O)c4c(O)ccc(C(F)(F)F)c4C[C@H]3C[C@@H]12]

Compounds S6-6-2 and S6-6-3 were prepared from compound S6-4 with HCHO by using General Procedures B-1, C and D-2. S6-6-2: ¹H NMR (400 MHz, CD₃OD, hydrochloride salt) δ 7.75 (d, J=9.2 Hz, 1H), 6.94 (d, J=9.2 Hz, 1H), 3.83 (br s, 1H), 3.19-3.15 (m, 1H), 3.06-2.98 (m, 1H), 2.91 (s, 3H), 2.82-2.79 (m, 1H), 2.51 (t, J=14.8 Hz, 1H), 2.20 (ddd, J=13.2, 5.2, 2.4 Hz, 1H), 1.60-1.51 (m, 1H); MS (ESI) m/z 469.30 (M+H). S6-6-3: ¹H NMR (400 MHz, CD₃OD, hydrochloride salt) δ 7.77 (d, J=9.2 Hz, 1H), 6.95 (d, J=9.2 Hz, 1H), 4.22 (br s, 0.5 H), 4.14 (br s, 0.5 H), 3.40-3.29 (m, 1H), 3.22-2.94 (m, 7H), 2.53 (t, J=14.8 Hz, 1H), 2.26-2.19 (m, 1H), 1.88-1.75 (m, 2H), 1.70-1.59 (m, 1H), 1.06-0.98 (m, 3H); MS (ESI) m/z 511.36 (M+H).

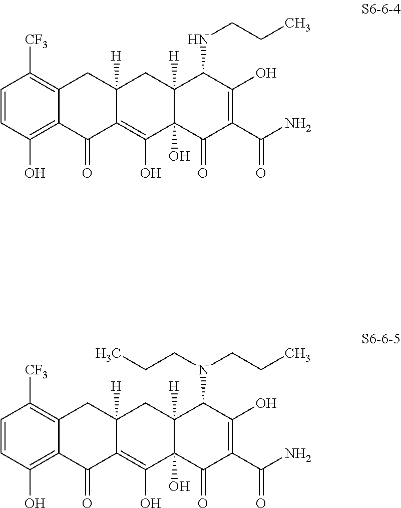 [SMILES: CCCN[C@@H]1C(O)=C(C(N)=O)C(=O)[C@@]2(O)C(O)=C3C(=O)c4c(O)ccc(C(F)(F)F)c4C[C@H]3C[C@@H]12 ; CCCN(CCC)[C@@H]1C(O)=C(C(N)=O)C(=O)[C@@]2(O)C(O)=C3C(=O)c4c(O)ccc(C(F)(F)F)c4C[C@H]3C[C@@H]12]

Compounds S6-6-4 and S6-6-5 were prepared from compound S6-2 by using General Procedures C and D-2. S6-6-4: ¹H NMR (400 MHz, CD₃OD, hydrochloride salt) δ 7.75 (d, J=9.2 Hz, 1H), 6.93 (d, J=9.2 Hz, 1H), 3.90 (s, 1H), 3.34-3.15 (m, 3H), 3.06-2.97 (m, 1H), 2.87 (d, J=12.4 Hz, 1H), 2.50 (t, J=14.8 Hz, 1H), 2.21 (ddd, J=14.0, 5.2, 2.8 Hz, 1H), 1.82-1.73 (m, 2H), 1.60-1.50 (m, 1H), 1.02 (t, J=7.2 Hz, 3H); MS (ESI) m/z 497.29 (M+H). S6-6-5: ¹H NMR (400 MHz, CD₃OD, hydrochloride salt) δ 7.77 (d, J=9.2 Hz, 1H), 6.96 (d, J=9.2 Hz, 1H), 4.24 (s, 1H), 3.51-3.46 (m, 1H), 3.41-3.26 (m, 2H), 3.23-3.03 (m, 3H), 2.95-2.92 (m, 1H), 2.54 (t, J=14.8 Hz, 1H), 2.20 (ddd, J=13.2, 4.4, 2.4 Hz, 1H), 1.89-1.79 (m, 4H), 1.68-1.59 (m, 1H), 1.03 (t, J=7.2 Hz, 3H), 0.99 (t, J=7.2 Hz, 3H); MS (ESI) m/z 539.38 (M+H).

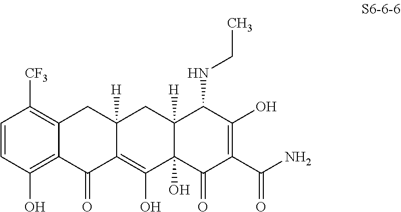 [SMILES: CCN[C@@H]1C(O)=C(C(N)=O)C(=O)[C@@]2(O)C(O)=C3C(=O)c4c(O)ccc(C(F)(F)F)c4C[C@H]3C[C@@H]12]

Compound S6-6-6 was prepared from compound S6-3 with CH₃CHO by using General Procedures B-1 (at 0° C.), C and D-2: ¹H NMR (400 MHz, CD₃OD, hydrochloride salt) δ 7.75 (d, J=9.2 Hz, 1H), 6.94 (d, J=9.2 Hz, 1H), 3.88 (s, 1H), 3.47-3.39 (m, 1H), 3.37-3.29 (m, 1H), 3.19-3.15 (m, 1H), 3.05-2.97 (m, 1H), 2.84 (d, J=12.4 Hz, 1H), 2.51 (t, J=14.8 Hz, 1H), 2.21 (ddd, J=13.6, 4.8, 2.4 Hz, 1H), 1.60-1.51 (m, 1H), 1.36 (t, J=7.6 Hz, 3H); MS (ESI) m/z 483.29 (M+H).

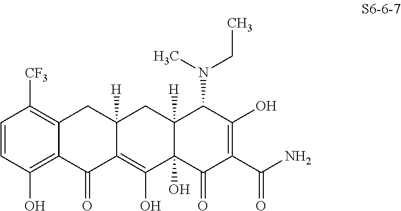 [SMILES: CCN(C)[C@@H]1C(O)=C(C(N)=O)C(=O)[C@@]2(O)C(O)=C3C(=O)c4c(O)ccc(C(F)(F)F)c4C[C@H]3C[C@@H]12]

Compound S6-6-7 was prepared from compound S6-3 with CH₃CHO by using General Procedures B-1 (at 0° C.), then B-1 again with HCHO, C and D-2: ¹H NMR (400 MHz, CD₃OD, hydrochloride salt) δ 7.76 (d, J=9.2 Hz, 1H), 6.95 (d, J=9.2 Hz, 1H), 4.25 (br s, 0.5 H), 4.16 (br s, 0.5 H), 3.52-3.43 (m, 1H), 3.39-3.31 (m, 1H), 3.22-3.18 (m, 5H), 2.53 (t, J=14.8 Hz, 1H), 2.27-2.20 (m, 1H), 1.70-1.58 (m, 1H), 1.43-1.36 (m, 3H); MS (ESI) m/z 497.32 (M+H).

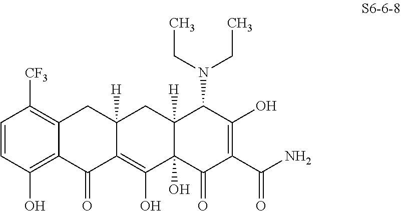 [SMILES: CCN(CC)[C@@H]1C(O)=C(C(N)=O)C(=O)[C@@]2(O)C(O)=C3C(=O)c4c(O)ccc(C(F)(F)F)c4C[C@H]3C[C@@H]12]

Compound S6-6-8 was prepared from compound S6-3 with CH₃CHO by using General Procedures B-1, C and D-2: ¹H NMR (400 MHz, CD₃OD, hydrochloride salt) δ 7.76 (d, J=9.2 Hz, 1H), 6.95 (d, J=9.2 Hz, 1H), 4.27 (s, 1H), 3.64-3.55 (m, 1H), 3.46 (q, J=7.6 Hz, 2 H), 3.36-3.29 (m, 1H), 3.22-3.17 (m, 1H), 3.11-3.03 (m, 1H), 2.93-2.90 (m, 1H), 2.53 (t, J=14.8 Hz, 1H), 2.22 (ddd, J=13.6, 5.2, 2.8 Hz, 1H), 1.68-1.59 (m, 1H), 1.41 (t, J=7.2 Hz, 3H), 1.40 (t, J=7.2 Hz, 3H); MS (ESI) m/z 511.34 (M+H).

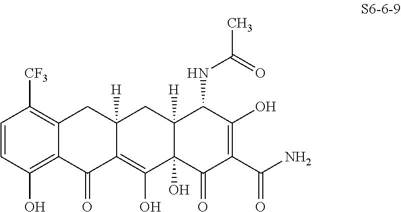 [SMILES: CC(=O)N[C@@H]1C(O)=C(C(N)=O)C(=O)[C@@]2(O)C(O)=C3C(=O)c4c(O)ccc(C(F)(F)F)c4C[C@H]3C[C@@H]12]

Compound S6-6-9 was prepared from compound S6-3 with Ac₂O by using General Procedures B-2, C and D-2: ¹H NMR (400 MHz, CD₃OD) δ 7.74 (d, J=9.2 Hz, 1H), 6.92 (d, J=9.2 Hz, 1H), 4.69 (d, J=6.4 Hz, 1H), 3.14-3.10 (m, 1H), 3.04-2.96 (m, 1H), 2.72 (t, J=14.8 Hz, 1H), 2.47-2.42 (m, 1H), 2.39-2.33 (m, 1H), 2.03 (s, 3H), 1.62-1.55 (m, 1H); MS (ESI) m/z 497.29 (M+H).

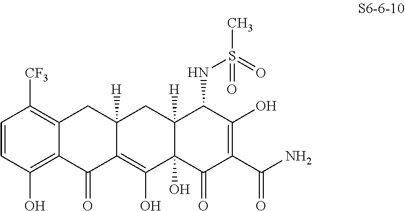 [SMILES: CS(=O)(=O)N[C@@H]1C(O)=C(C(N)=O)C(=O)[C@@]2(O)C(O)=C3C(=O)c4c(O)ccc(C(F)(F)F)c4C[C@H]3C[C@@H]12]

Compound S6-6-10 was prepared from compound S6-3 with Ms₂O by using General Procedures B-2, C and D-2: ¹H NMR (400 MHz, CD₃OD) δ 7.73 (d, J=9.2 Hz, 1H), 6.91 (d, J=9.2 Hz, 1H), 4.10 (d, J=4.4 Hz, 1H), 3.19-3.14 (m, 1H), 3.14 (s, 3H), 3.04-2.96 (m, 1 H), 2.70 (t, J=14.8 Hz, 1H), 2.51 (dt, J=14.0, 4.0 Hz, 1H), 2.27 (ddd, J=14.0, 6.4, 3.6 Hz, 1H), 1.69-1.61 (m, 1H); MS (ESI) m/z 533.32 (M+H).

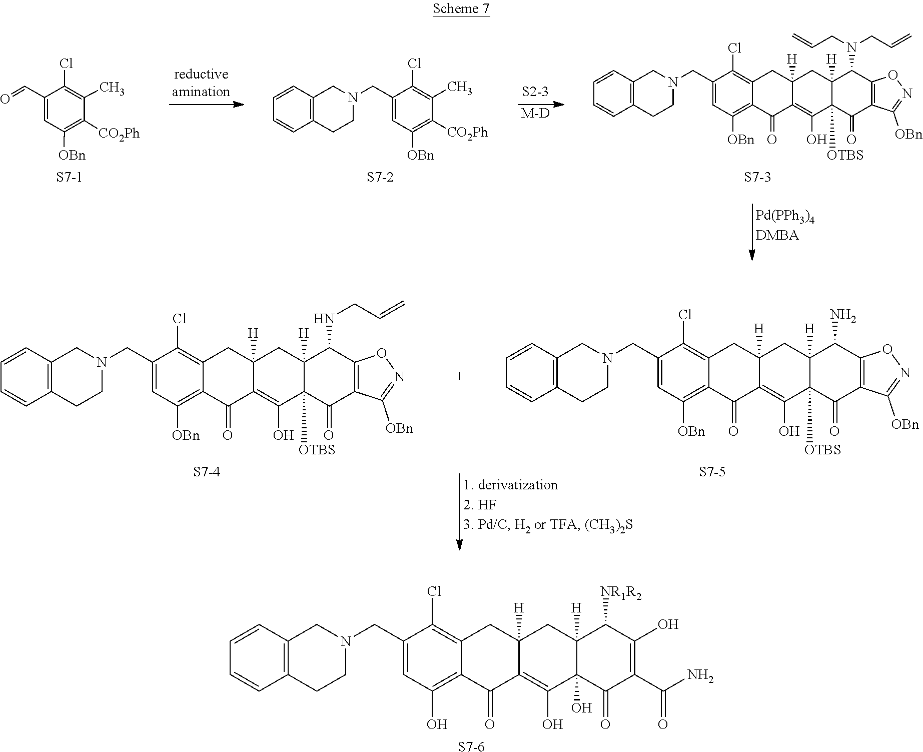

The following compounds were prepared per Scheme 7.

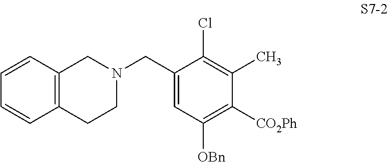

Compound S7-2 was prepared from compound S7-1 (prepared according to literature procedures including J. Med. Chem., 2013, 56, 8112-8138) and isoquinoline by using General Procedure B-1: ¹H NMR (400 MHz, CDCl₃) δ 7.38-7.22 (m, 9H), 7.14-7.08 (m, 5H), 7.00-6.99 (m, 1H), 5.13 (br s, 2H), 3.78 (br s, 2H), 3.70 (br s, 2H), 2.87 (br s, 2H), 2.74 (br s, 2 H), 2.48 (s, 3H); MS (ESI) m/z 498.5 (M+H).

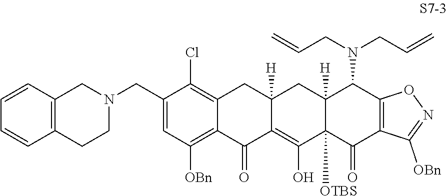 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc(CN5CCc6ccccc6C5)c(Cl)c4C[C@H]3C[C@@H]12]

Compound S7-3 was prepared from compound S7-2 and diallyenone S2-3 by using General Procedure E: ¹H NMR (400 MHz, CDCl₃) δ 15.96 (br s, 1H), 7.51-7.49 (m, 2H), 7.40-7.31 (m, 5H), 7.27-7.20 (m, 4H), 7.16-7.12 (m, 3H), 6.98-6.96 (m, 1H), 5.86-5.76 (m, 2H), 5.36 (s, 2H), 5.23-5.16 (m, 4H), 5.12-5.10 (m, 2H), 4.09 (d, J=9.6 Hz, 1H), 3.74-3.65 (m, 4H), 3.37-3.31 (m, 4H), 3.23-3.17 (m, 2H), 3.02-2.94 (m, 1H), 2.84-2.70 (m, 4H), 2.52-2.42 (m, 2H), 2.15-2.12 (m, 1H), 0.83 (s, 9H), 0.26 (s, 3H), 0.14 (s, 3H); MS (ESI) m/z 938.70 (M+H).

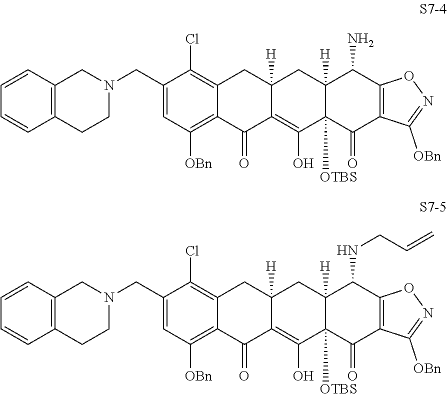 [SMILES: CC(C)(C)[Si](C)(C)O[C@@]12C(=O)c3c(OCc4ccccc4)noc3[C@@H](N)[C@@H]1C[C@@H]1Cc3c(Cl)c(CN4CCc5ccccc5C4)cc(OCc4ccccc4)c3C(=O)C1=C2O ; C=CCN[C@@H]1c2onc(OCc3ccccc3)c2C(=O)[C@@]2(O[Si](C)(C)C(C)(C)C)C(O)=C3C(=O)c4c(OCc5ccccc5)cc(CN5CCc6ccccc6C5)c(Cl)c4C[C@H]3C[C@@H]12]

Compounds S7-4 and S7-5 were prepared from compound S7-3 by using General Procedure A. S7-4: MS (ESI) m/z 858.59 (M+H). S7-5: MS (ESI) m/z 898.71 (M+H).

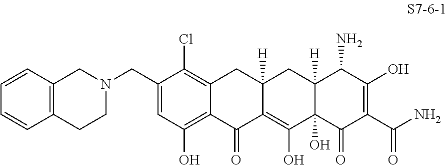 [SMILES: NC(=O)C1=C(O)[C@@H](N)[C@@H]2C[C@@H]3Cc4c(Cl)c(CN5CCc6ccccc6C5)cc(O)c4C(=O)C3=C(O)[C@]2(O)C1=O]

Compound S7-6-1 was prepared from compound S7-4 by using General Procedures C and D-1: ¹H NMR (400 MHz, CD₃OD, dihydrochloride salt) δ 7.33-7.25 (m, 4H), 7.19 (d, J=7.2 Hz, 1H), 4.73, 4.68 (ABq, J=13.6 Hz, 2H), 4.55 (s, 2H), 3.92 (s, 1H), 3.84 (br s, 1H), 3.62 (br s, 1H), 3.42 (dd, J=16.0, 4.4 Hz, 1H), 3.30-3.18 (m, 2H), 3.09-3.02 (m, 1H), 2.72-2.69 (m, 1H), 2.42 (t, J=14.8 Hz, 1H), 2.29 (ddd, J=14.0, 5.2, 2.4 Hz, 1H), 1.65-1.55 (m, 1H); MS (ESI) m/z 566.35 (M+H).

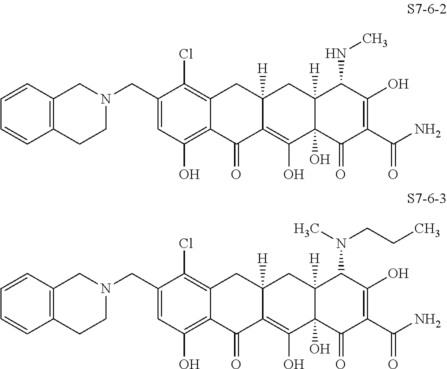

Compounds S7-6-2 and S7-6-3 were prepared from compound S7-5 with HCHO by using General Procedures B-1, C and D-2. S7-6-2: ¹H NMR (400 MHz, CD₃OD, dihydrochloride salt) δ 7.33-7.26 (m, 4H), 7.19 (d, J=7.2 Hz, 1H), 4.72, 4.67 (ABq, J=13.2 Hz, 2H), 4.55 (s, 2H), 3.85 (br s, 2H), 3.63 (br s, 1H), 3.42 (dd, J=16.0, 4.0 Hz, 1H), 3.30-3.22 (m, 2H), 3.10-3.04 (m, 1H), 2.92 (s, 3H), 2.85 (d, J=12.6 Hz, 1H), 2.43 (t, J=14.8 Hz, 1H), 2.29-2.23 (m, 1H), 1.64-1.54 (m, 1H); MS (ESI) m/z 580.4 (M+H). S7-6-3: ¹H NMR (400 MHz, CD₃OD, dihydrochloride salt) δ 7.33-7.26 (m, 4H), 7.21-7.19 (m, 1H), 4.72, 4.68 (ABq, J=15.6 Hz, 2H), 4.55 (s, 2H), 4.24 (s, 0.5 H), 4.17 (s, 0.5 H), 3.84 (br s, 1H), 3.62 (br s, 1H), 3.46-3.34 (m, 2H), 3.32-2.96 (m, 8H), 2.44 (br t, J=15.2 Hz, 1H), 2.99 (br t, J=13.2 Hz, 1H), 1.86-1.77 (m, 2H), 1.68-1.65 (m, 1H), 1.05-0.99 (m, 3H); MS (ESI) m/z 622.4 (M+H).

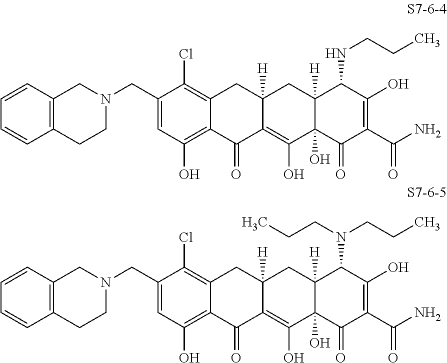 [SMILES: CCCN[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12 ; CCCN(CCC)[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12]

Compounds S7-6-4 and S7-6-5 were prepared from compound S7-3 by using General Procedures C and D-2. S7-6-4: ¹H NMR (400 MHz, CD₃OD, dihydrochloride salt) δ 7.31-7.18 (m, 5H), 4.71 (q, J=13.6 Hz, 2H), 4.55 (s, 2H), 3.93 (s, 1H), 3.84 (br s, 1H), 3.63 (br s, 1 H), 3.42-3.38 (m, 1H), 3.38-3.17 (m, 4H), 3.07 (br s, 1H), 2.95 (d, J=12.8 Hz, 1H), 2.39 (t, J=14.4 Hz, 1H), 2.29 (d, J=12.0 Hz, 1H), 1.83-1.74 (m, 2H), 1.61-1.52 (m, 1H), 1.03 (t, J=7.6 Hz, 3H); MS (ESI) m/z 608.43 (M+H). S7-6-5: ¹H NMR (400 MHz, CD₃OD, dihydrochloride salt) δ 7.34-7.19 (m, 5H), 4.70 (s, 2H), 4.55 (s, 2H), 4.26 (s, 1H), 3.87-3.85 (m, 1H), 3.63 (br s, 1H), 3.54-3.37 (m, 3H), 3.29-3.13 (m, 5H), 2.99 (d, J=13.2 Hz, 1H), 2.44 (t, J=14.4 Hz, 1H), 2.27 (d, J=12.0 Hz, 1H), 1.90-1.80 (m, 4H), 1.71-1.61 (m, 1H), 1.05-0.98 (m, 6H); MS (ESI) m/z 650.45 (M+H).

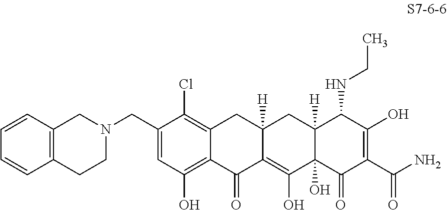 [SMILES: CCN[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12]

Compound S7-6-6 was prepared from compound S7-4 with CH₃CHO by using General Procedures B-1 (at 0° C.), C and D-1: ¹H NMR (400 MHz, CD₃OD, dihydrochloride salt) δ 7.33-7.18 (m, 5H), 4.73, 4.67 (ABq, J=13.6 Hz, 2H), 4.55 (s, 2H), 3.90 (s, 1H), 3.84 (br s, 1H), 3.62 (br s, 1H), 3.48-3.32 (m, 3H), 3.29-3.21 (m, 2H), 3.10-3.03 (m, 1H), 2.90 (d, J=12.8 Hz, 1H), 2.41 (t, J=14.4 Hz, 1H), 2.30-2.26 (m, 1H), 1.63-1.53 (m, 1H), 1.37 (t, J=7.6 Hz, 3H); MS (ESI) m/z 594.40 (M+H).

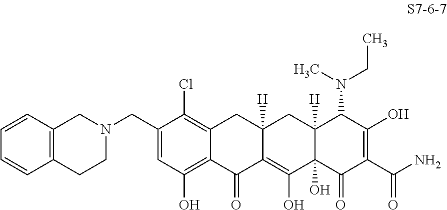

Compound S7-6-7 was prepared from compound S7-4 with CH₃CHO by using General Procedure B-1 (at 0° C.), then B-1 again with HCHO, C and D-1: ¹H NMR (400 MHz, CD₃OD, dihydrochloride salt) δ 7.33-7.26 (m, 4H), 7.21-7.19 (m, 1H), 4.73, 4.68 (ABq, J=13.2 Hz, 2H), 4.55 (s, 2H), 4.26 (s, 0.5 H), 4.18 (s, 0.5 H), 3.85 (br s, 1H), 3.62 (br s, 1H), 3.56-3.34 (m, 3H), 3.30-3.14 (m, 3H), 3.04-2.95 (m, 4H), 2.42 (br t, J=15.2 Hz, 1H), 2.30 (br t, J=15.2 Hz, 1H), 1.73-1.61 (m, 1H), 1.44-1.37 (m, 3H); MS (ESI) m/z 608.43 (M+H).

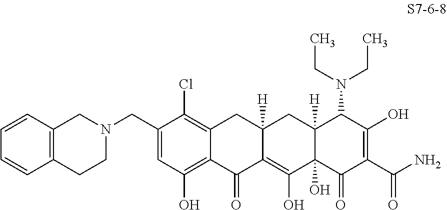

Compound S7-6-8 was prepared from compound S7-4 with CH₃CHO by using General Procedures B-1, C and D-1: ¹H NMR (400 MHz, CD₃OD, dihydrochloride salt) δ 7.34-7.25 (m, 4H), 7.20-7.18 (m, 1H), 4.74, 4.68 (ABq, J=13.2 Hz, 2H), 4.55 (s, 2H), 4.28 (s, 1H), 3.84 (br s, 1H), 3.65-3.56 (m, 2H), 3.53-3.34 (m, 4H), 3.29-3.10 (m, 3H), 2.98 (d, J=13.2 Hz, 1H), 2.41 (t, J=14.8 Hz, 1H), 2.30 (br d, J=12.4 Hz, 1H), 1.71-1.64 (m, 1H), 1.43 (t, J=7.2 Hz, 3H), 1.42 (t, J=7.2 Hz, 3H); MS (ESI) m/z 622.42 (M+H).

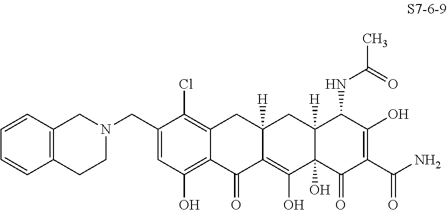 [SMILES: CC(=O)N[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12]

Compound S7-6-9 was prepared from compound S7-4 with Ac₂O by using General Procedures B-2, C and D-1: ¹H NMR (400 MHz, CD₃OD, hydrochloride salt) δ 7.33-7.24 (m, 4H), 7.21-7.19 (m, 1H), 4.72-4.65 (m, 3H), 4.55 (s, 2H), 3.84 (br s, 1H), 3.61 (br s, 1H), 3.37-3.33 (m, 1H), 3.30-3.20 (m, 2H), 3.05-2.99 (m, 1H), 2.63 (t, J=15.2 Hz, 1H), 2.46-2.36 (m, 2H), 2.05 (s, 3H), 1.66-1.59 (m, 1H); MS (ESI) m/z 608.42 (M+H).

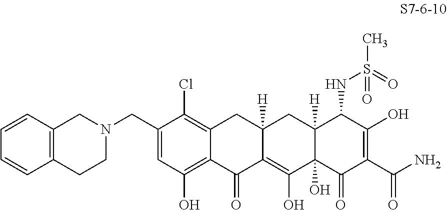 [SMILES: CS(=O)(=O)N[C@@H]1C(O)=C(C(N)=O)C(=O)[C@@]2(O)C(O)=C3C(=O)c4c(O)cc(CN5CCc6ccccc6C5)c(Cl)c4C[C@H]3C[C@@H]12]

Compound S7-6-10 was prepared from compound S7-4 with Ms₂O by using General Procedures B-2, C and D-1: ¹H NMR (400 MHz, CD₃OD, hydrochloride salt) δ 7.32-7.23 (m, 4H), 7.20-7.18 (m, 1H), 4.69 (s, 2H), 4.54 (s, 2H), 4.10 (d, J=4.4 Hz, 1H), 3.84 (br s, 1H), 3.63 (br s, 1H), 3.38 (dd, J=16.8, 5.2 Hz, 1H), 3.28-3.20 (m, 2H), 3.16 (s, 3H), 2.99-2.91 (m, 1H), 2.60 (t, J=16.0 Hz, 1H), 2.48-2.44 (m, 1H), 2.32-2.26 (m, 1H), 1.72-1.64 (m, 1 H); MS (ESI) m/z 644.36 (M+H).

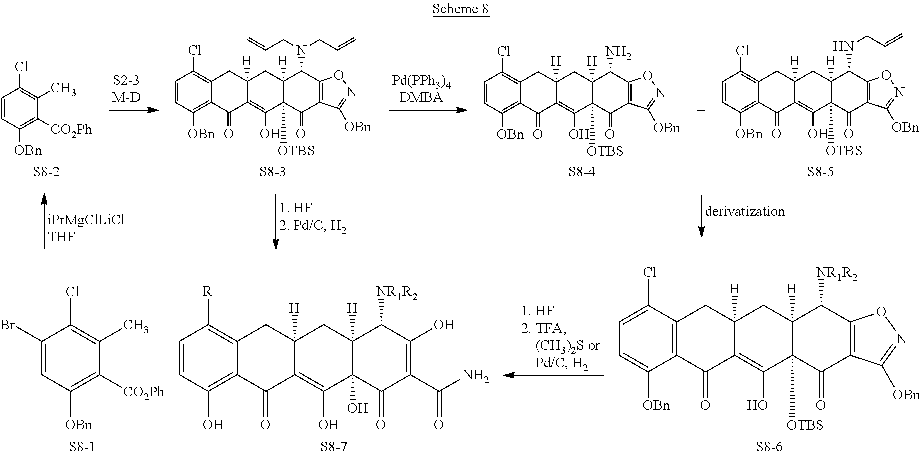

The following compounds were prepared per Scheme 8.

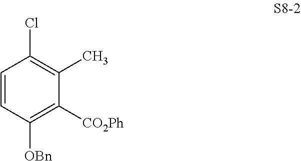

Compound S8-1 (1.62 g, 3.76 mmol, 1 eq, prepared per literature procedures including J. Med. Chem., 2013, 56, 8112-8138) was dissolved in THF (16 mL). The resulting reaction solution was cooled to −78° C. A solution of ⁱPrMgCl.LiCl (1.3 M, 4.89 mL, 4.89 mmol, 1.3 eq) was added. The resulting reaction solution was then stirred in an ice/water bath for 2 h and saturated NH₄Cl solution was added. The resulting reaction mixture was extracted with EtOAc. The organic phase was separated, washed with brine and concentrated. The residue was purified by flash column chromatography (100 g silica gel, 2→8% EtOAc/hexanes) to give compound S8-2 as a white solid (1.1 g, 83%): ¹H NMR (400 MHz, CDCl₃) δ 7.43-7.34 (m, 8 H), 7.26-7.23 (m, 1H), 7.10-7.08 (m, 2H), 6.83-6.80 (m, 1H), 5.13 (s, 2H), 2.45 (s, 3H).

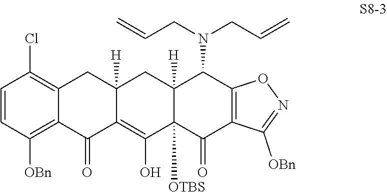 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)ccc(Cl)c4C[C@H]3C[C@@H]12]

Compound S8-3 was prepared from compound S8-2 and diallyenone S2-3 by using General Procedure E: MS (ESI) m/z 793.60 (M+H).

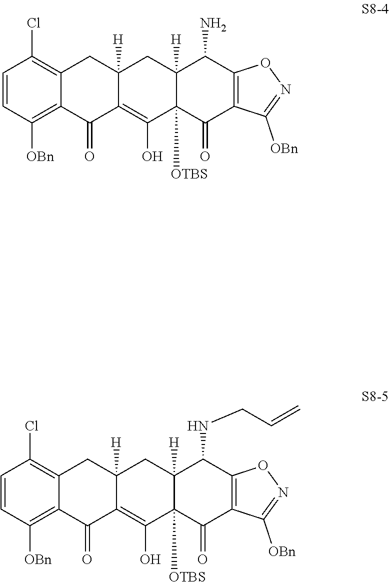 [SMILES: CC(C)(C)[Si](C)(C)O[C@@]12C(=O)c3c(OCc4ccccc4)noc3[C@@H](N)[C@@H]1C[C@@H]1Cc3c(Cl)ccc(OCc4ccccc4)c3C(=O)C1=C2O ; C=CCN[C@@H]1c2onc(OCc3ccccc3)c2C(=O)[C@@]2(O[Si](C)(C)C(C)(C)C)C(O)=C3C(=O)c4c(OCc5ccccc5)ccc(Cl)c4C[C@H]3C[C@@H]12]

Compounds S8-4 and S8-5 were prepared from compound S8-3 by using General Procedure A. S8-4: MS (ESI) m/z 713.45 (M+H). S8-5: MS (ESI) m/z 753.51 (M+H).

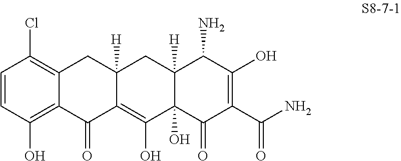 [SMILES: NC(=O)C1=C(O)[C@@H](N)[C@@H]2C[C@@H]3Cc4c(Cl)ccc(O)c4C(=O)C3=C(O)[C@]2(O)C1=O]

Compound S8-7-1 was prepared from compound S8-4 by using General Procedures C and D-1: ¹H NMR (400 MHz, CD₃OD, hydrochloride salt) δ 7.49 (d, J=8.8 Hz, 1H), 6.83 (d, J=8.8 Hz, 1H), 3.90 (s, 1H), 3.32-3.27 (s, 1H), 3.10-2.94 (m, 1H), 2.66-2.62 (m, 1H), 2.34 (t, J=15.6 Hz, 1H), 2.23 (ddd, J=13.6, 5.2, 2.8 Hz, 1H), 1.63-1.54 (m, 1H); MS (ESI) m/z 421.24 (M+H).

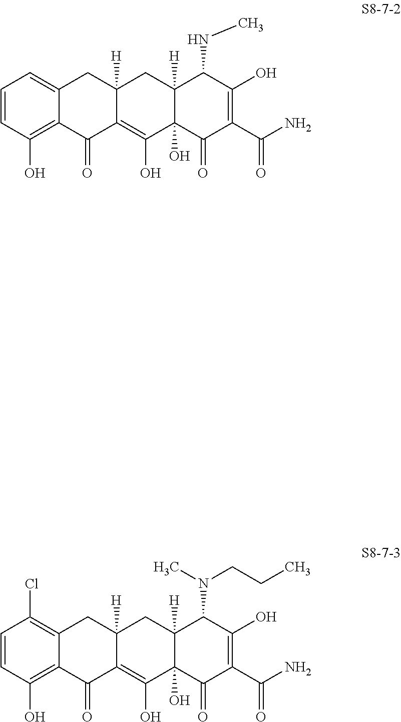 [SMILES: CN[C@@H]1C(O)=C(C(N)=O)C(=O)[C@@]2(O)C(O)=C3C(=O)c4c(O)cccc4C[C@H]3C[C@@H]12 ; CCCN(C)[C@@H]1C(O)=C(C(N)=O)C(=O)[C@@]2(O)C(O)=C3C(=O)c4c(O)ccc(Cl)c4C[C@H]3C[C@@H]12]

Compounds S8-7-2 and S8-7-3 were prepared from compound S8-5 with HCHO by using General Procedures B-1, C and D-2. S8-7-2: ¹H NMR (400 MHz, CD₃OD, hydrochloride salt) δ 7.40 (dd, J=8.4, 7.2 Hz, 1H), 6.79 (d, J=8.4 Hz, 1H), 6.73 (d, J=7.2 Hz, 1H), 3.79 (s, 1H), 3.04-2.95 (m, 1H), 2.90 (s, 3H), 2.87-2.82 (m, 1H), 2.77-2.74 (m, 1H), 2.54 (t, J=14.8 Hz, 1H), 2.15 (ddd, J=13.2, 4.8, 2.8 Hz, 1H), 1.56-1.47 (m, 1H); MS (ESI) m/z 401.29 (M+H). S8-7-3: ¹H NMR (400 MHz, CD₃OD, hydrochloride salt) δ 7.50 (d, J=9.2 Hz, 1H), 6.84 (d, J=9.2 Hz, 1H), 4.22 (s, 0.5 H), 4.13 (s, 0.5 H), 3.41-3.32 (m, 2H), 3.22-3.15 (m, 1 H), 3.09-2.91 (m, 5H), 2.34 (t, J=15.2 Hz, 1H), 2.26-2.19 (m, 1H), 1.88-1.74 (m, 2H), 1.68-1.62 (m, 1H), 1.06-0.99 (m, 3H); MS (ESI) m/z 477.33 (M+H).

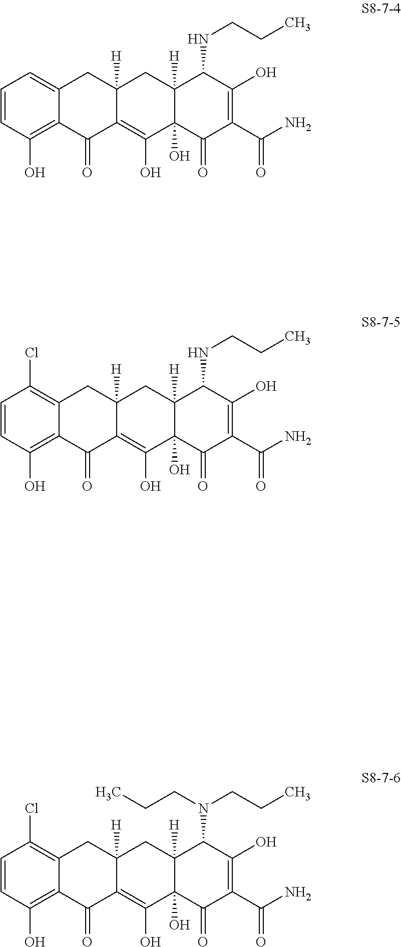 [SMILES: CCCN[C@@H]1C(O)=C(C(N)=O)C(=O)[C@@]2(O)C(O)=C3C(=O)c4c(O)cccc4C[C@H]3C[C@@H]12 ; CCCN[C@@H]1C(O)=C(C(N)=O)C(=O)[C@@]2(O)C(O)=C3C(=O)c4c(O)ccc(Cl)c4C[C@H]3C[C@@H]12 ; CCCN(CCC)[C@@H]1C(O)=C(C(N)=O)C(=O)[C@@]2(O)C(O)=C3C(=O)c4c(O)ccc(Cl)c4C[C@H]3C[C@@H]12]

Compounds S8-7-4, S8-7-5 and S8-7-6 were prepared from compound S8-3 by using General Procedures C and D-2. S8-7-4: ¹H NMR (400 MHz, CD₃OD, hydrochloride salt) δ 7.40 (dd, J=8.8, 7.2 Hz, 1H), 6.79 (d, J=8.8 Hz, 1H), 6.73 (d, J=7.2 Hz, 1H), 3.86 (s, 1 H), 3.33-3.17 (m, 2H), 3.03-2.94 (m, 1H), 2.87-2.80 (m, 1H), 2.53 (t, J=14.4 Hz, 1H), 2.17 (ddd, J=13.2, 4.8, 2.4 Hz, 1H), 1.82-1.72 (m, 2H), 1.56-1.47 (m, 1H), 1.03 (t, J=7.6 Hz, 3 H); MS (ESI) m/z 429.34 (M+H). S8-7-5: ¹H NMR (400 MHz, CD₃OD, hydrochloride salt) δ 7.48 (d, J=8.8 Hz, 1H), 6.82 (d, J=8.8 Hz, 1H), 3.88 (s, 1H), 3.34-3.18 (m, 2H), 3.03-2.94 (m, 1H), 2.85 (d, J=12.8 Hz, 1H), 2.30 (t, J=15.2 Hz, 1H), 2.24-2.20 (m, 1H), 1.82-1.72 (m, 2H), 1.60-1.50 (m, 1H), 1.02 (t, J=7.6 Hz, 3H); MS (ESI) m/z 463.31 (M+H). S8-7-6: ¹H NMR (400 MHz, CD₃OD, hydrochloride salt) δ 7.50 (d, J=9.2 Hz, 1H), 6.84 (d, J=9.2 Hz, 1H), 4.24 (s, 1H), 3.53-3.45 (m, 1H), 3.41-3.25 (m, 3H), 3.22-3.16 (m, 1H), 3.09-2.99 (m, 1H), 2.95-2.92 (m, 1H), 2.33 (t, J=14.8 Hz, 1H), 2.21 (ddd, J=13.2, 4.4, 2.8 Hz, 1H), 1.89-1.74 (m, 4H), 1.68-1.59 (m, 1H), 1.03 (t, J=7.6 Hz, 3H), 0.99 (t, J=7.6 Hz, 3H); MS (ESI) m/z 505.35 (M+H).

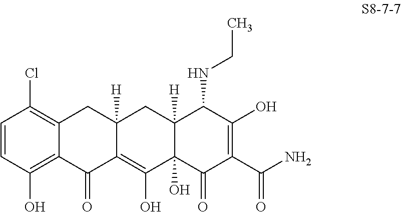 [SMILES: CCN[C@@H]1C(O)=C(C(N)=O)C(=O)[C@@]2(O)C(O)=C3C(=O)c4c(O)ccc(Cl)c4C[C@H]3C[C@@H]12]

Compound S8-7-7 was prepared from compound S8-4 with CH₃CHO by using General Procedures B-1 (at 0° C.), C and D-1: ¹H NMR (400 MHz, CD₃OD, hydrochloride salt) δ 7.48 (d, J=8.4 Hz, 1H), 6.82 (d, J=8.4 Hz, 1H), 3.88 (s, 1H), 3.46-3.41 (m, 1H), 3.37-3.32 (m, 1H), 3.30-3.25 (m, 1H), 3.03-2.95 (m, 1H), 2.85-2.82 (m, 1H), 2.30 (t, J=15.2 Hz, 1H), 2.24-2.20 (m, 1H), 1.60-1.51 (m, 1H), 1.36 (t, J=7.6 Hz, 3H); MS (ESI) m/z 449.26 (M+H).

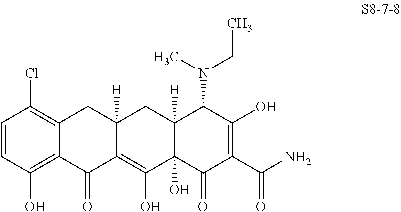 [SMILES: CCN(C)[C@@H]1C(O)=C(C(N)=O)C(=O)[C@@]2(O)C(O)=C3C(=O)c4c(O)ccc(Cl)c4C[C@H]3C[C@@H]12]

Compound S8-7-8 was prepared from compound S8-4 with CH₃CHO by using General Procedure B-1 (at 0° C.), then B-1 again with HCHO, C and D-1: ¹H NMR (400 MHz, CD₃OD, hydrochloride salt) δ 7.50 (d, J=8.8 Hz, 1H), 6.84 (d, J=8.8 Hz, 1H), 4.23 (s, 0.5 H), 4.14 (s, 0.5 H), 3.51-3.43 (m, 1H), 3.37-3.30 (m, 2H), 3.08-2.89 (m, 5H), 2.34 (t, J=15.2 Hz, 1 H), 2.28-2.19 (m, 1H), 1.71-1.58 (m, 1H), 1.42 (t, J=7.2 Hz, 1.5 H), 1.38 (t, J=7.2 Hz, 1.5 H); MS (ESI) m/z 463.28 (M+H).

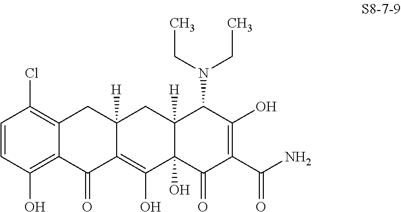 [SMILES: CCN(CC)[C@@H]1C(O)=C(C(N)=O)C(=O)[C@@]2(O)C(O)=C3C(=O)c4c(O)ccc(Cl)c4C[C@H]3C[C@@H]12]

Compound S8-7-9 was prepared from compound S8-4 with CH₃CHO by using General Procedures B-1, C and D-1: ¹H NMR (400 MHz, CD₃OD, hydrochloride salt) δ 7.50 (d, J=8.8 Hz, 1H), 6.84 (d, J=8.8 Hz, 1H), 4.23 (s, 1H), 3.65-3.56 (m, 1H), 3.50-3.44 (m, 2H), 3.36-3.29 (m, 2H), 3.08-3.01 (m, 1H), 2.93-2.90 (m, 1H), 2.36-2.23 (m, 2H), 1.69-1.59 (m, 1H), 1.42 (t, J=7.6 Hz, 6H), 0.99 (t, J=7.6 Hz, 3H); MS (ESI) m/z 477.30 (M+H).

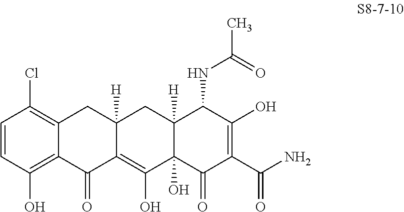 [SMILES: CC(=O)N[C@@H]1C(O)=C(C(N)=O)C(=O)[C@@]2(O)C(O)=C3C(=O)c4c(O)ccc(Cl)c4C[C@H]3C[C@@H]12]

Compound S8-7-10 was prepared from compound S8-4 with Ac₂O by using General Procedures B-2, C and D-1: ¹H NMR (400 MHz, CD₃OD) δ 7.47 (d, J=9.2 Hz, 1H), 6.80 (d, J=9.2 Hz, 1H), 4.68 (d, J=6.4 Hz, 1H), 3.22 (dd, J=16.0, 4.4 Hz, 1H), 3.01-2.93 (m, 1H), 2.52 (t, J=15.6 Hz, 1H), 2.46-2.42 (m, 1H), 2.39-2.32 (m, 1H), 2.04 (s, 3H), 1.64-1.56 (m, 1H); MS (ESI) m/z 463.27 (M+H).

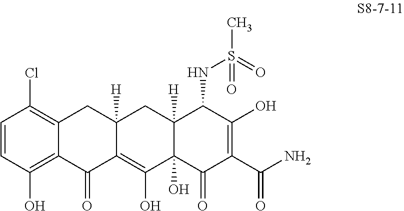 [SMILES: CS(=O)(=O)N[C@@H]1C(O)=C(C(N)=O)C(=O)[C@@]2(O)C(O)=C3C(=O)c4c(O)ccc(Cl)c4C[C@H]3C[C@@H]12]

Compound S8-7-11 was prepared from compound S8-4 with Ms₂O by using General Procedures B-2, C and D-1: ¹H NMR (400 MHz, CD₃OD) δ 7.46 (d, J=9.2 Hz, 1H), 6.79 (d, J=9.2 Hz, 1H), 4.10 (d, J=4.4 Hz, 1H), 3.25 (dd, J=16.0, 4.4 Hz, 1H), 3.14 (s, 3H), 3.01-2.92 (m, 1H), 2.53-2.48 (m, 2H), 2.30-2.24 (m, 1H), 1.69-1.61 (m, 1H); MS (ESI) m/z 499.22 (M+H).

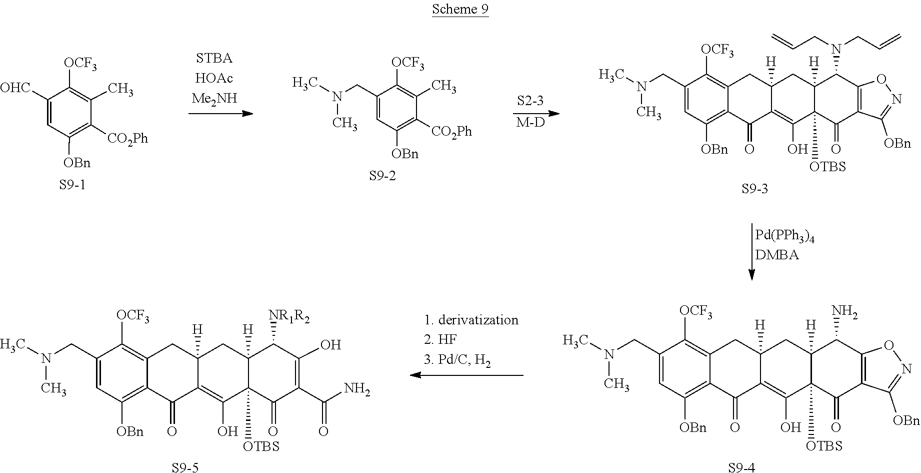

The following compounds were prepared per Scheme 9.

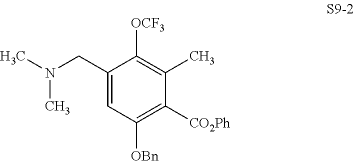

Compound S9-1 (0.15 g, 0.35 mmol, 1.0 eq, prepared per literature procedures including WO 2014036502 A2) was dissolved in DCM (2 mL). Dimethylamine (0.12 mL, 5.6 M in EtOH, 0.70 mmol, 2.0 eq) and acetic acid (60 μL, 1.14 mmol, 3.0 eq) were added under a nitrogen atmosphere. Then sodium triacetoxyborohydride (148 mg, 0.70 mmol, 2.0 eq) was added. After 10 min, LC/MS indicated that the starting material was consumed. Saturated NaHCO₃ solution was added and extracted with DCM. The organic phase was concentrated under reduced pressure. The residue was purified by flash column chromatography (Biotage 10 g silica gel column, 10%→30% EtOAc in hexanes gradient), yielding 100 mg (62%) of the compound S9-2 as a colorless oil: ¹H NMR (400 MHz, CDCl₃) δ 7.45-7.43 (m, 2H), 7.38-7.34 (m, 5H), 7.26-7.22 (m, 1H), 7.20 (s, 1H), 7.09-7.06 (m, 2H), 5.17 (s, 2H), 3.49 (s, 2H), 2.40 (s, 3H), 2.23 (s, 6H); MS (ESI) m/z 460.23 (M+H).

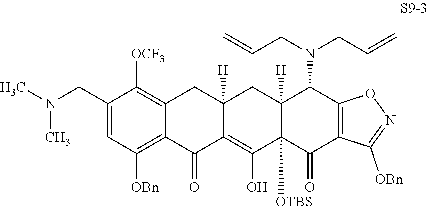 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc(CN(C)C)c(OC(F)(F)F)c4C[C@H]3C[C@@H]12]

Compound S9-3 was prepared from compound S9-2 and diallyenone S2-3 by using General Procedure E: MS (ESI) m/z 900.41 (M+H).

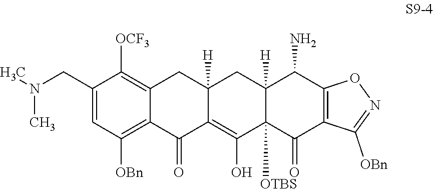 [SMILES: CN(C)Cc1cc(OCc2ccccc2)c2c(c1OC(F)(F)F)C[C@H]1C[C@H]3[C@H](N)c4onc(OCc5ccccc5)c4C(=O)[C@@]3(O[Si](C)(C)C(C)(C)C)C(O)=C1C2=O]

Compounds S9-4 was prepared from compound S9-3 by using General Procedure A: ¹H NMR (400 MHz, CDCl₃) δ 16.52 (s, 1H), 7.49-7.44 (m, 6H), 7.41-7.29 (m, 6H), 7.25 (s, 1H), 5.40, 5.36 (ABq, J=12.0 Hz, 2H), 5.31, 5.22 (ABq, J=12.0 Hz, 2H), 3.92 (d, J=2.0 Hz, 1H), 3.49, 3.43 (ABq, J=14.4 Hz, 2H), 3.02 (dd, J=16.0, 4.4 Hz, 1H), 2.79-2.71 (m, 1 H), 2.64-2.61 (m, 1H), 2.28-2.20 (m, 1H), 2.20 (s, 6H), 2.13-2.08 (m, 1H), 1.58-1.49 (m, 1 H), 0.74 (s, 9H), 0.22 (s, 3 H), 0.10 (s, 3H); MS (ESI) m/z 820.33 (M+H).

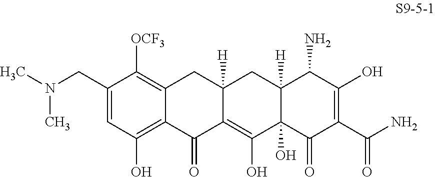 [SMILES: CN(C)Cc1cc(O)c2c(c1OC(F)(F)F)C[C@H]1C[C@H]3[C@H](N)C(O)=C(C(N)=O)C(=O)[C@@]3(O)C(O)=C1C2=O]

Compound S9-5-1 was prepared from compound S9-4 by using General Procedures C and D-2: ¹H NMR (400 MHz, CD₃OD, dihydrochloride salt) δ 7.24 (s, 1H), 4.45 (s, 2H), 3.90 (s, 1H), 3.19 (dd, J=15.6, 3.6 Hz, 1H), 3.04-2.96 (m, 1H), 2.94 (s, 3H), 2.86 (s, 3H), 2.68 (br d, J=12.8 Hz, 1H), 2.41 (t, J=14.4 Hz, 1H), 2.27-2.24 (m, 1H), 1.64-1.54 (m, 1H); MS (ESI) m/z 528.18 (M+H).

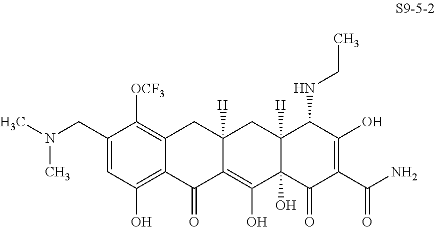 [SMILES: CCN[C@@H]1C(O)=C(C(N)=O)C(=O)[C@@]2(O)C(O)=C3C(=O)c4c(O)cc(CN(C)C)c(OC(F)(F)F)c4C[C@H]3C[C@@H]12]

Compound S9-5-2 was prepared from compound S9-4 with CH₃CHO by using General Procedures B-1 (at 0° C.), C and D-2: ¹H NMR (400 MHz, CD₃OD, dihydrochloride salt) δ 7.20 (s, 1H), 4.45 (s, 2H), 3.88 (s, 1H), 3.46-3.39 (m, 1H), 3.37-3.30 (m, 1H), 3.18 (dd, J=15.6, 4.4 Hz, 1H), 3.05-2.97 (m, 1H), 2.94 (s, 3H), 2.86 (s, 3H), 2.86-2.83 (m, 1H), 2.41 (t, J=14.8 Hz, 1H), 2.24 (ddd, J=14.0, 5.6, 2.8 Hz, 1H), 1.64-1.54 (m, 1H), 1.36 (t, J=7.2 Hz, 3 H); MS (ESI) m/z 556.2 (M+H).

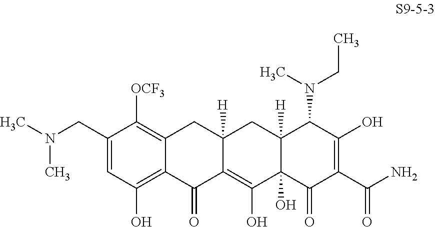 [SMILES: CCN(C)[C@@H]1C(O)=C(C(N)=O)C(=O)[C@@]2(O)C(O)=C3C(=O)c4c(O)cc(CN(C)C)c(OC(F)(F)F)c4C[C@H]3C[C@@H]12]

Compound S9-5-3 was prepared from compound S9-4 with CH₃CHO by using General Procedures B-1 (at 0° C.), B-1 again with HCHO, C and D-2: ¹H NMR (400 MHz, CD₃OD, dihydrochloride salt) δ 7.22 (s, 1H), 4.46 (s, 2H), 4.24 (s, 0.5 H), 4.15 (s, 0.5 H), 3.53-3.44 (m, 1H), 3.38-3.30 (m, 1H), 3.22-3.18 (m, 1H), 3.11-2.94 (m, 8H), 2.86 (s, 3H), 2.42 (t, J=14.4 Hz, 1H), 2.29-2.23 (m, 1H), 1.68-1.60 (m, 1H), 1.44-1.34 (m, 3H); MS (ESI) m/z 570.2 (M+H).

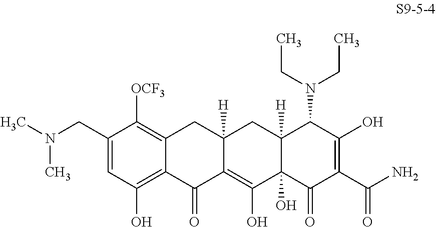 [SMILES: CCN(CC)[C@@H]1C(O)=C(C(N)=O)C(=O)[C@@]2(O)C(O)=C3C(=O)c4c(O)cc(CN(C)C)c(OC(F)(F)F)c4C[C@H]3C[C@@H]12]

Compound S9-5-4 was prepared from compound S9-4 with CH₃CHO by using General Procedures B-1, C and D-2: ¹H NMR (400 MHz, CD₃OD, dihydrochloride salt) δ 7.28 (s, 1 H), 4.47 (s, 2H), 4.28 (s, 1H), 3.65-3.56 (m, 1H), 3.54-3.43 (m, 2H), 3.41-3.34 (m, 1H), 3.21 (br d, J=15.6 Hz, 1H), 3.13-3.05 (m, 1H), 2.99-2.96 (m, 1H), 2.96 (s, 3H), 2.86 (s, 3 H), 2.41 (t, J=14.8 Hz, 1H), 2.28 (br d, J=12.8 Hz, 1H), 1.69-1.60 (m, 1H), 1.42 (t, J=7.2 Hz, 6H); MS (ESI) m/z 584.20 (M+H).

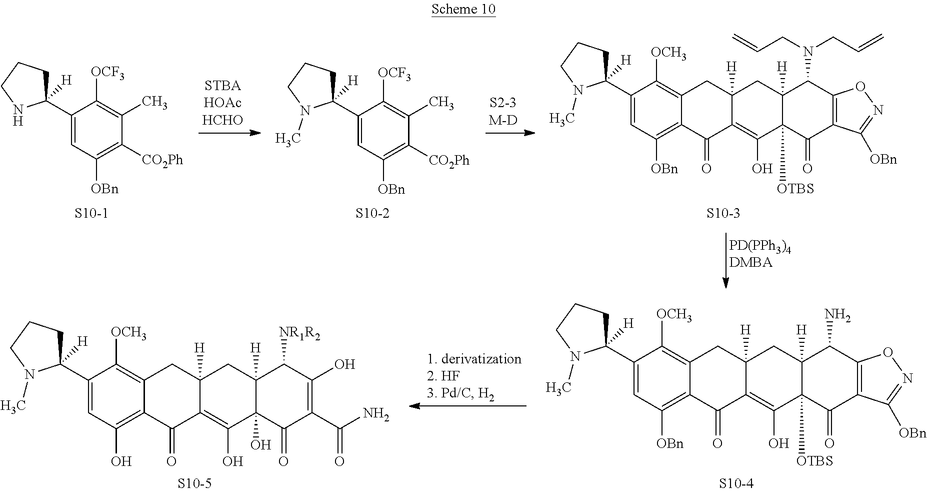

The following compounds were prepared per Scheme 10.

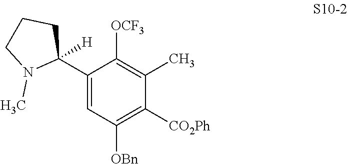

Compound S10-2 was prepared from S10-1 (prepared according to literature procedures including WO 2014036502 A2) with HCHO by using General Procedure B-1: ¹H NMR (400 MHz, CDCl₃) δ 7.46-7.44 (m, 2H), 7.38-7.33 (m, 5H), 7.26 (s, 1H), 7.26-7.22 (m, 1H), 7.09-7.06 (m, 2H), 5.19, 5.15 (ABq, J=11.6 Hz, 2H), 3.49 (t, J=8.4 Hz, 1H), 3.26-3.21 (m, 1H), 2.33 (q, J=9.2 Hz, 1H), 2.29-2.20 (m, 1H), 2.15 (s, 3H), 1.97-1.88 (m, 1H), 1.86-1.78 (m, 1H), 1.60-1.50 (m, 1H); MS (ESI) m/z 486.15 (M+H).

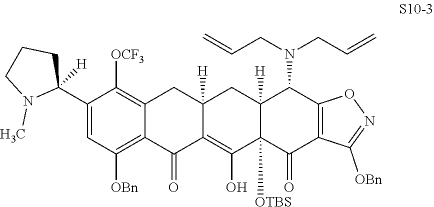 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)cc([C@@H]5CCCN5C)c(OC(F)(F)F)c4C[C@H]3C[C@@H]12]

Compound S10-3 was prepared from compound S10-2 and diallyenone S2-3 by using General Procedure E: ¹H NMR (400 MHz, CDCl₃) δ 15.99 (s, 1H), 7.51-7.47 (m, 4H), 7.40-7.31 (m, 5H), 7.28-7.26 (m, 2H), 5.83-5.73 (m, 2H), 5.36 (s, 2H), 5.23 (s, 2H), 5.23-5.18 (m, 2H), 5.09 (d, J=10.4 Hz, 2H), 4.09 (d, J=10.4 Hz, 1H), 3.43 (t, J=8.0 Hz, 1H), 3.35-3.30 (m, 2H), 3.22-3.16 (m, 3H), 3.12 (dd, J=15.2, 4.0 Hz, 1H), 2.95-2.88 (m, 1H), 2.66 (t, J=15.6 Hz, 1H), 2.52-2.48 (m, 1H), 2.45-2.40 (m, 1H), 2.30 (q, J=8.4 Hz, 1H), 2.23-2.10 (m, 1H), 2.06 (s, 3H), 1.96-1.89 (m, 1H), 1.85-1.77 (m, 1H), 1.59-1.51 (m, 1H), 0.82 (s, 9 H), 0.25 (s, 3H), 0.13 (s, 3H); MS (ESI) m/z 926.37 (M+H).

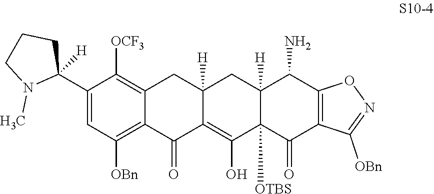 [SMILES: CN1CCC[C@H]1c1cc(OCc2ccccc2)c2c(c1OC(F)(F)F)C[C@H]1C[C@H]3[C@H](N)c4onc(OCc5ccccc5)c4C(=O)[C@@]3(O[Si](C)(C)C(C)(C)C)C(O)=C1C2=O]

Compound S10-4 was prepared from compound S10-3 by using General Procedure A: ¹H NMR (400 MHz, CDCl₃) δ 16.51 (s, 1H), 7.55-7.53 (m, 2H), 7.49-7.47 (m, 2H), 7.41-7.28 (m, 7H), 5.40, 5.36 (ABq, J=12.4 Hz, 2H), 5.28, 5.22 (ABq, J=12.0 Hz, 2H), 3.92 (d, J=2.4 Hz, 1H), 3.43 (t, J=8.0 Hz, 1H), 3.23-3.19 (m, 1H), 3.02 (dd, J=15.2, 3.6 Hz, 1H), 2.80-2.71 (m, 1H), 2.64-2.61 (m, 1H), 2.34-2.10 (m, 3H), 2.09 (s, 3H), 1.96-1.79 (m, 3H), 1.58-1.49 (m, 2H), 0.74 (s, 9H), 0.22 (s, 3H), 0.10 (s, 3H); MS (ESI) m/z 846.37 (M+H).

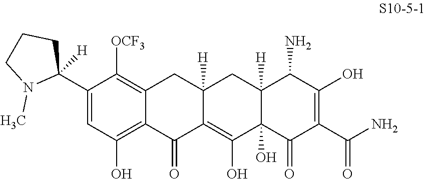 [SMILES: CN1CCC[C@H]1c1cc(O)c2c(c1OC(F)(F)F)C[C@H]1C[C@H]3[C@H](N)C(O)=C(C(N)=O)C(=O)[C@@]3(O)C(O)=C1C2=O]

Compound S10-5-1 was prepared from compound S10-4 by using General Procedures C and D-2: ¹H NMR (400 MHz, CD₃OD, dihydrochloride salt) δ 7.27 (s, 1H), 4.85-4.74 (m, 1H), 3.88 (s, 1H), 3.88-3.83 (m, 1H), 3.42-3.33 (m, 1H), 3.21 (dd, J=16.0, 3.6 Hz, 1H), 3.03-2.94 (m, 1H), 2.77 (s, 3H), 2.66-2.54 (m, 2H), 2.54-2.23 (m, 5H), 1.65-1.55 (m, 1H); MS (ESI) m/z 554.14 (M+H).

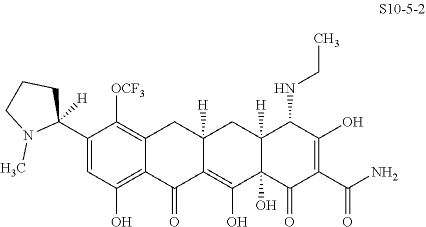 [SMILES: CCN[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12]

Compound S10-5-2 was prepared from compound S10-4 with CH₃CHO by using General Procedures B-1 (at 0° C.), C and D-2: ¹H NMR (400 MHz, CD₃OD, dihydrochloride salt) δ 7.33 (s, 1H), 4.82-4.75 (m, 1H), 3.89 (s, 1H), 3.89-3.83 (m, 1H), 3.47-3.33 (m, 3H), 3.21 (dd, J=16.0, 4.0 Hz, 1H), 3.06-2.98 (m, 1H), 2.87 (d, J=12.8 Hz, 1H), 2.77 (s, 3H), 2.61-2.52 (m, 1H), 2.43-2.44 (m, 5H), 1.64-1.54 (m, 1H), 1.37 (t, J=7.2 Hz, 3H); MS (ESI) m/z 582.16 (M+H).

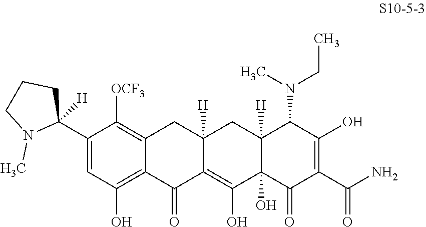 [SMILES: CCN(C)[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12]

Compound S9-5-3 was prepared from compound S9-4 with CH₃CHO by using General Procedures B-1 (at 0° C.), B-1 again with HCHO, C and D-2: ¹H NMR (400 MHz, CD₃OD, dihydrochloride salt) δ 7.38 (s, 1H), 4.80-4.75 (m, 1H), 4.26 (s, 0.5 H), 4.18 (s, 0.5 H), 3.89-3.85 (m, 1H), 3.56-3.46 (m, 1H), 3.43-3.32 (m, 2H), 3.23 (d, J=15.6 Hz, 1H), 3.13-2.95 (m, 5H), 2.77 (s, 3H), 2.62-2.55 (m, 1H), 2.44-2.26 (m, 5H), 1.70-1.60 (m, 1H), 1.44-1.37 (m, 3H); MS (ESI) m/z 596.18 (M+H).

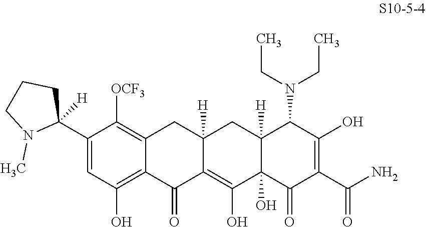 [SMILES: CCN(CC)[C@@H]1C(O)=C(C(N)=O)C(=O)[C@@]2(O)C(O)=C3C(=O)c4c(O)cc([C@@H]5CCCN5C)c(OC(F)(F)F)c4C[C@H]3C[C@@H]12]

Compound S9-5-4 was prepared from compound S9-4 with CH₃CHO by using General Procedures B-1, C and D-2: ¹H NMR (400 MHz, CD₃OD, dihydrochloride salt) δ 7.40 (s, 1 H), 4.79-4.77 (m, 1H), 4.27 (s, 1H), 3.89-3.86 (m, 1H), 3.63-3.56 (m, 1H), 3.48-3.35 (m, 4 H), 3.23 (br d, J=15.6 Hz, 1H), 3.09 (br s, 1H), 2.97 (br d, J=13.6 Hz, 1H), 2.76 (s, 3H), 2.60-2.54 (m, 1H), 2.42-2.27 (m, 5H), 1.68-1.60 (m, 1H), 1.41 (t, J=6.4 Hz, 6H); MS (ESI) m/z 610.19 (M+H).

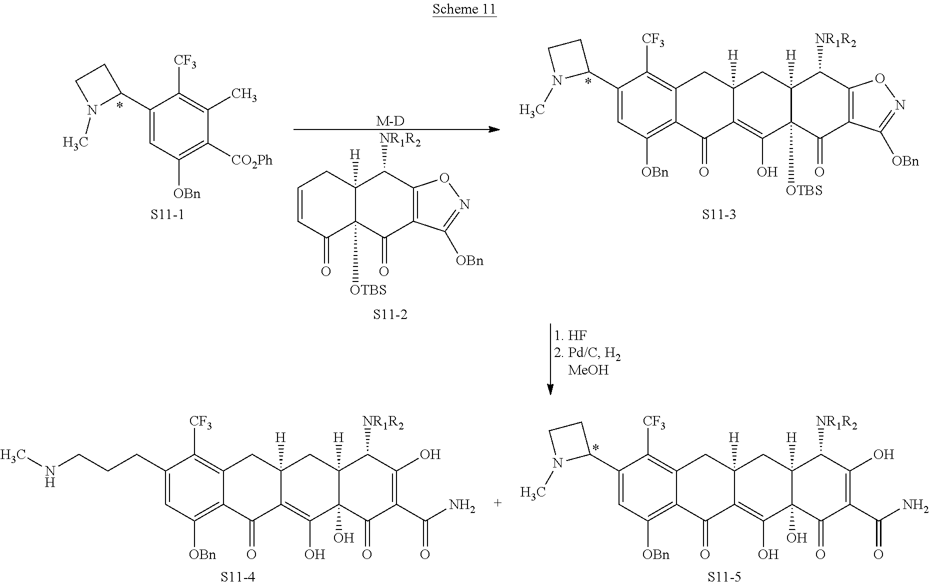

The following compounds were prepared per Scheme 11.

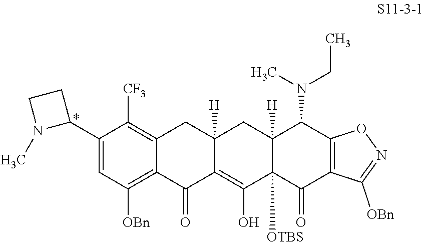 [SMILES: CCN(C)[C@@H]1c2onc(OCc3ccccc3)c2C(=O)[C@@]2(O[Si](C)(C)C(C)(C)C)C(O)=C3C(=O)c4c(OCc5ccccc5)cc(C5CCN5C)c(C(F)(F)F)c4C[C@H]3C[C@@H]12]

Compound S11-3-1 was prepared from S11-1 (prepared according to literature procedures including WO 2012021712 A1) and C-4 methylethylaminoenone S11-2-1 (prepared according to literature procedures including WO 2014036502 A2) by using General Procedure E: ¹H NMR (400 MHz, CDCl₃) δ 15.84 (s, 1H), 7.59 (s, 1H), 7.51-7.49 (m, 4H), 7.39-7.32 (m, 5H), 7.28-7.24 (m, 1H), 5.39, 5.34 (ABq, J=12.8 Hz, 2H), 5.36 (s, 2H), 4.24 (br s, 1H), 4.02 (d, J=9.6 Hz, 1H), 3.43-3.39 (m, 1H), 3.20 (d, J=15.6 Hz, 1H), 2.94-2.80 (m, 3H), 2.74-2.60 (m, 2H), 2.56-2.44 (m, 3H), 3.36 (s, 3H), 2.26-2.14 (m, 1H), 2.21 (s, 3 H), 1.97-1.90 (m, 1H), 1.05 (t, J=7.2 Hz, 3H), 0.84 (s, 9H), 0.28 (s, 3H), 0.16 (s, 3H); MS (ESI) m/z 858.3 (M+H).

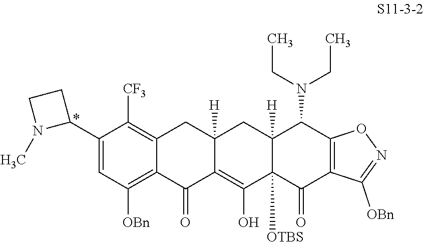 [SMILES: CCN(CC)[C@@H]1c2onc(OCc3ccccc3)c2C(=O)[C@@]2(O[Si](C)(C)C(C)(C)C)C(O)=C3C(=O)c4c(OCc5ccccc5)cc(C5CCN5C)c(C(F)(F)F)c4C[C@H]3C[C@@H]12]

Compound S11-3-2 was prepared from S11-1 and C-4 diethylaminoenone S11-2-2 (prepared according to literature procedures including WO 2014036502 A2) by using General Procedure E: ¹H NMR (400 MHz, CDCl₃) δ 15.83 (s, 1H), 7.60 (s, 1H), 7.51-7.47 (m, 4H), 7.39-7.31 (m, 5H), 7.28-7.24 (m, 1H), 5.42-5.30 (m, 4H), 4.24-4.19 (m, 1H), 4.03 (d, J=10.4 Hz, 1H), 3.42-3.38 (m, 1H), 3.23-3.19 (m, 1H), 2.95-2.86 (m, 2H), 2.75-2.68 (m, 5H), 2.51-2.44 (m, 3H), 2.23-2.20 (m, 1H), 2.20 (s, 3H), 1.97-1.90 (m, 1H), 1.08 (t, J=7.2 Hz, 3 H), 0.84 (s, 9H), 0.28 (s, 3H), 0.16 (s, 3H); MS (ESI) m/z 872.3 (M+H).

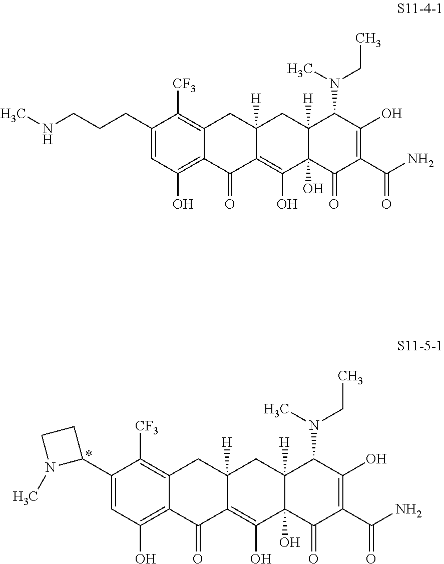 [SMILES: CCN(C)[C@@H]1C(O)=C(C(N)=O)C(=O)[C@@]2(O)C(O)=C3C(=O)c4c(O)cc(CCCNC)c(C(F)(F)F)c4C[C@H]3C[C@@H]12 ; CCN(C)[C@@H]1C(O)=C(C(N)=O)C(=O)[C@@]2(O)C(O)=C3C(=O)c4c(O)cc(C5CCN5C)c(C(F)(F)F)c4C[C@H]3C[C@@H]12]

Compounds S11-4-1 and S11-5-1 were prepared from compound S11-3-1 by using General Procedures C and D-2. S11-4-1: ¹H NMR (400 MHz, CD₃OD, dihydrochloride salt) S 6.89 (s, 1H), 4.16 (s, 1H), 3.39 (br s, 2H), 3.29-3.22 (m, 1H), 3.08-2.86 (m, 9H), 2.70 (s, 3 H), 2.53 (t, J=15.1 Hz, 1H), 2.21-2.18 (m, 1H), 2.02-1.92 (m, 2H), 1.67-1.61 (m, 1H), 1.37 (t, J=7.3 Hz, 3H); MS (ESI) m/z 568.18 (M+H). S11-5-1: ¹H NMR (400 MHz, CD₃OD, dihydrochloride salt) δ 7.05 (s, 1H), 5.94 (t, J=8.2 Hz, 1H), 4.17-4.10 (m, 3H), 3.40 (br s, 2 H), 3.22-3.18 (m, 1H), 3.12-2.90 (m, 8H), 2.72-2.58 (m, 3H), 2.24-2.21 (m, 1H), 1.69-1.60 (m, 1H), 1.39 (t, J=7.3 Hz, 3H); MS (ESI) m/z 566.16 (M+H).

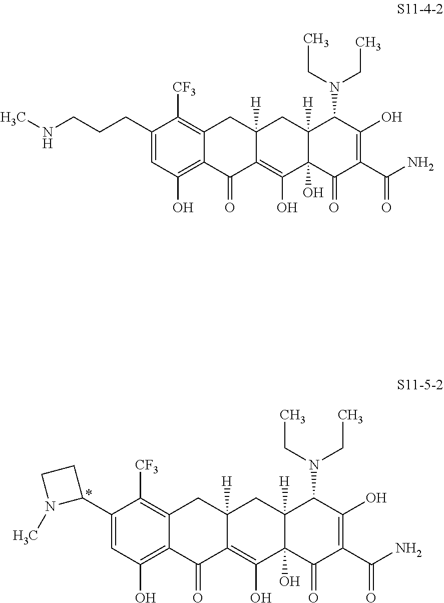 [SMILES: CCN(CC)[C@@H]1C(O)=C(C(N)=O)C(=O)[C@@]2(O)C(O)=C3C(=O)c4c(O)cc(CCCNC)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc(C5CCN5C)c(C(F)(F)F)c4C[C@H]3C[C@@H]12]

Compounds S11-4-2 and S11-5-2 were prepared from compound S11-3-1 by using General Procedures C and D-2. S11-4-2: ¹H NMR (400 MHz, CD₃OD, dihydrochloride salt) S 6.89 (s, 1H), 4.24 (s, 1H), 3.53-3.47 (m, 2H), 3.42-3.34 (m, 2H), 3.27-3.22 (m, 1H), 3.08-3.04 (m, 2H), 2.99-2.86 (m, 4H), 2.70 (s, 3H), 2.53 (t, J=15.2 Hz, 1H), 2.20 (ddd, J=14.0, 5.2, 2.8 Hz, 1H), 2.00-1.93 (m, 2H), 1.67-1.57 (m, 1H), 1.40 (t, J=7.2 Hz, 6H); MS (ESI) m/z 582.2 (M+H). S11-5-2: ¹H NMR (400 MHz, CD₃OD, dihydrochloride salt) δ 7.05 (s, 1H), 5.94 (t, J=8.2 Hz, 1H), 4.24-4.10 (m, 3H), 3.51 (br s, 2H), 3.40 (br s, 2H), 3.23-3.19 (m, 1 H), 3.12-2.89 (m, 6H), 2.72-2.54 (m, 2H), 2.22 (ddd, J=13.7, 4.6, 2.7 Hz, 1H), 1.68-1.59 (m, 1H), 1.40 (t, J=7.3 Hz, 6H); MS (ESI) m/z 580.2 (M+H).

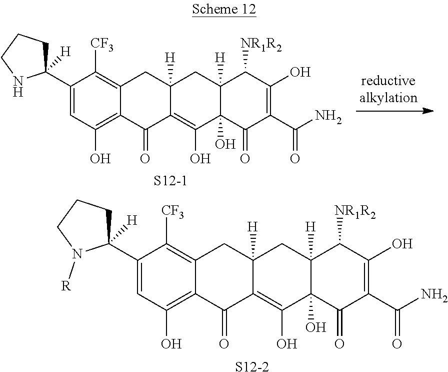

The following compounds were prepared per Scheme 12.

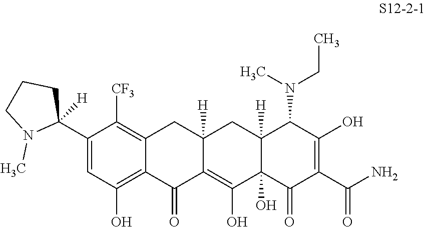 [SMILES: CCN(C)[C@@H]1C(O)=C(C(N)=O)C(=O)[C@@]2(O)C(O)=C3C(=O)c4c(O)cc([C@@H]5CCCN5C)c(C(F)(F)F)c4C[C@H]3C[C@@H]12]

To a solution of compound S12-1-1 (R₁,R₂═CH₃,CH₃CH₂, 26 mg, 0.041 mmol, 1 eq, prepared per literature procedures including WO 2014036502 A2) in CH₃OH (1 mL) was added HCHO solution (9 μL, 0.12 mmol, 3.0 eq). Pd—C (10 wt %, 10 mg) was added under nitrogen. The reaction vessel was sealed and purged with hydrogen by briefly evacuating the flask followed by flushing with hydrogen gas (1 atm). The reaction mixture was stirred under a hydrogen atmosphere (1 atm) at rt overnight. The reaction was filtered through a small Celite pad. The cake was washed with CH₃OH. The filtrate was concentrated. The residue was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 NHCl/water; Solvent B: CH₃CN; injection volume: 3.0 mL (0.05 N HCl/water); gradient: 5→35% B in A over 20 min; mass-directed fraction collection]. Fractions containing the desired product were collected and freeze-dried to yield compound S12-2-1 (15.6 mg): ¹H NMR (400 MHz, CD₃OD, dihydrochloride salt) δ 7.49 (s, 1H), 4.75 (t, J=8.0 Hz, 1H), 4.26 (s, 0.5 H), 4.18 (s, 0.5 H), 3.94-3.89 (m, 1H), 3.55-3.48 (m, 1H), 3.43-3.26 (m, 3H), 3.04-2.95 (m, 5H), 2.75-2.61 (m, 5H), 2.36-2.24 (m, 4H), 1.70-1.61 (m, 1H), 1.42-1.389 (m, 3H); MS (ESI) m/z 580.23 (M+H).

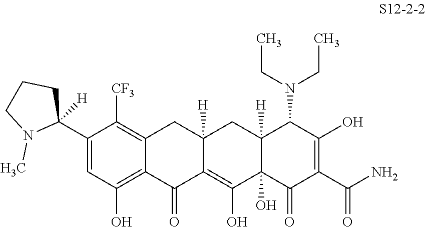 [SMILES: CCN(CC)[C@@H]1C(O)=C(C(N)=O)C(=O)[C@@]2(O)C(O)=C3C(=O)c4c(O)cc([C@@H]5CCCN5C)c(C(F)(F)F)c4C[C@H]3C[C@@H]12]

Compound S12-2-2 was prepared from compound S12-1-2 (R₁R₂=Et₂, prepared according to literature procedures including WO 2014036502 A2) by using a similar procedure for compound S12-2-1: ¹H NMR (400 MHz, CD₃OD, dihydrochloride salt) δ 7.50 (s, 1H), 4.74 (t, J=8.0 Hz, 1H), 4.26 (s, 1H), 3.94-3.89 (m, 1H), 3.64-3.56 (m, 1H), 3.53-3.45 (m, 2 H), 3.42-3.34 (m, 2H), 3.29-3.26 (m, 1H), 3.06-2.96 (m, 2H), 2.74 (s, 3H), 2.71-2.61 (m, 5 H), 2.36-2.22 (m, 4H), 1.69-1.59 (m, 1H), 1.41 (t, J=7.2 Hz, 6H); MS (ESI) m/z 594.06 (M+H).

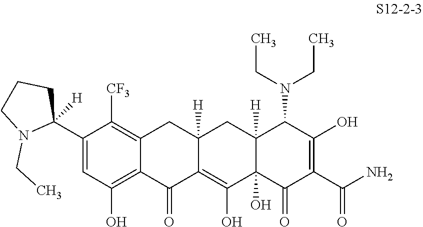 [SMILES: CCN(CC)[C@@H]1C(O)=C(C(N)=O)C(=O)[C@@]2(O)C(O)=C3C(=O)c4c(O)cc([C@@H]5CCCN5CC)c(C(F)(F)F)c4C[C@H]3C[C@@H]12]

Compound S12-2-3 was prepared from compound S12-1-2 (R₁R₂=Et₂) and CH₃CHO by using a similar procedure for compound S12-2-1: ¹H NMR (400 MHz, CD₃OD, dihydrochloride salt) δ 7.56 (s, 1H), 4.75 (t, J=8.0 Hz, 1H), 4.26 (s, 1H), 3.99-3.93 (m, 1 H), 3.63-3.56 (m, 1H), 3.53-3.43 (m, 2H), 3.39-3.32 (m, 2H), 3.29-3.25 (m, 1H), 3.10-2.95 (m, 4H), 2.70-2.62 (m, 2H), 2.34-2.20 (m, 4H), 1.69-1.59 (m, 1H), 1.41 (t, J=7.2 Hz, 6H), 1.24 (t, J=7.2 Hz, 3H); MS (ESI) m/z 608.07 (M+H).

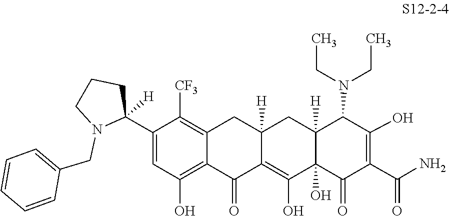 [SMILES: CCN(CC)[C@@H]1C(O)=C(C(N)=O)C(=O)[C@@]2(O)C(O)=C3C(=O)c4c(O)cc([C@@H]5CCCN5Cc5ccccc5)c(C(F)(F)F)c4C[C@H]3C[C@@H]12]

To a solution of compound S12-1-2 (R₁R₂=Et₂, 266 mg, 0.41 mmol, 1 eq) in CH₃OH (3 mL) was added PhCHO (100 μL, 0.99 mmol, 2.4 eq) and NaBH(OAc)₃ (110 mg, 0.52 mmol, 1.3 eq) at 0° C. The resulting reaction mixture was stirred at 0° C. for 15 min. Then the cold was removed and the reaction was stirred at rt for 15 min. Concentrated HCl (4 drops) was added and the resulting reaction was concentrated to ˜2 mL. The residue was dropped into stirring MTBE (70 mL) to give a suspension. The solid was collected by filtration, dried under vacuum. Then the solid was dissolved in 0.05 NHCl/water. The resulting solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 NHCl/water; Solvent B: CH₃CN; injection volume: 3.0 mL (0.05 NHCl/water); gradient: 10→60% B in A over 20 min; mass-directed fraction collection]: ¹H NMR (400 MHz, CD₃OD, dihydrochloride salt) δ 7.32-7.31 (m, 6H), 4.89 (t, J=8.0 Hz, 1H), 4.47 (d, J=12.8 Hz, 1H), 4.27 (s, 1H), 4.22 (d, J=12.8 Hz, 1H), 3.88-3.83 (m, 1H), 3.64-3.37 (m, 5H), 3.19-3.15 (m, 1H), 3.03-2.95 (m, 2H), 2.77-2.68 (m, 1H), 2.57 (t, J=14.8 Hz, 1H), 2.24-2.12 (m, 4H), 1.67-1.58 (m, 1H), 1.43 (t, J=7.2 Hz, 6H); MS (ESI) m/z 670.32 (M+H).

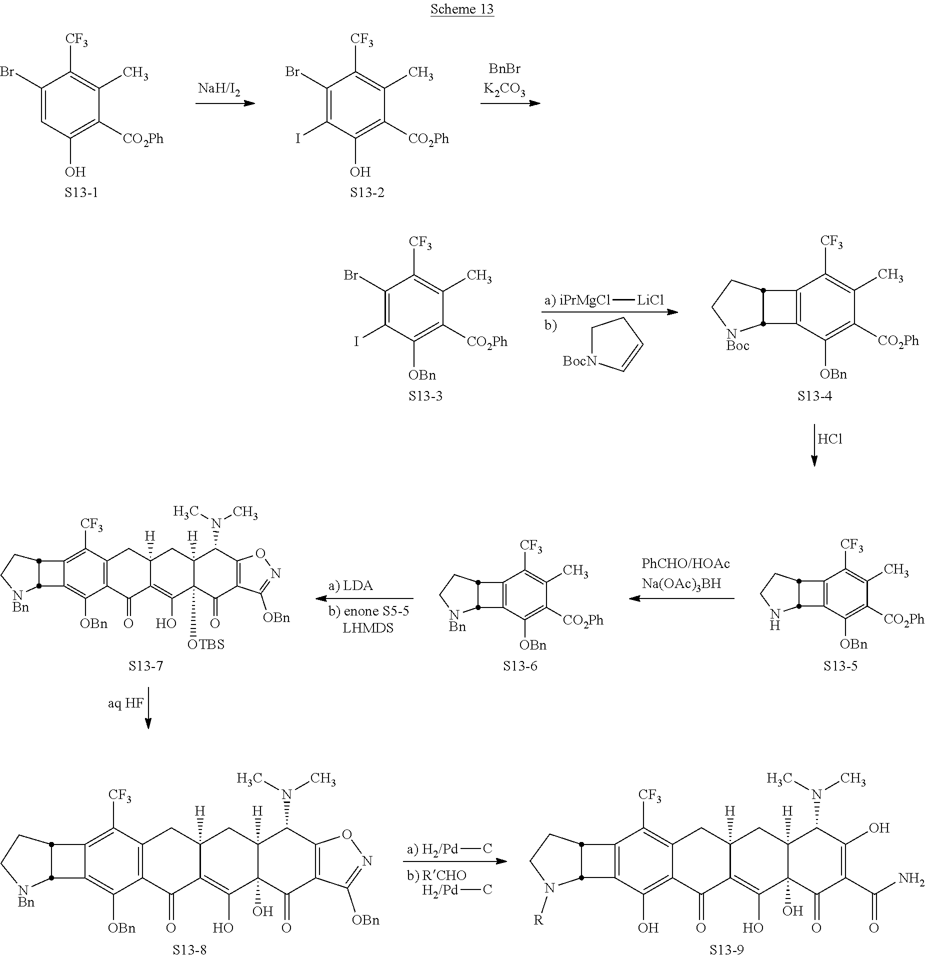

The following compounds were prepared per Scheme 13.

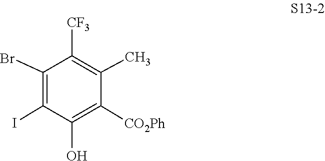

To compound S13-1 (1.04 g, 2.77 mmol, 1 eq) in toluene (8 mL) was added NaH (444 mg, 60% in mineral oil, 11.09 mmol, 4 eq). The white suspension was stirred at rt for 8 min.

Iodine (2.81 g, 11.09 mmol, 4 eq) was added. The reaction mixture was stirred at rt for overnight. Water and 1 N HCl (11 mL) were added, followed by the addition of 10% aqueous Na₂SO₃. The mixture was extracted with EtOAc. The organic phase was washed with brine and concentrated under reduced pressure to give the desired product S13-2: MS (ESI) m/z 498.9 (M−H).

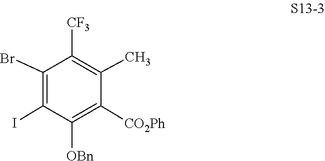

The above product S13-2 (2.77 mmol, crude, 1 eq) was dissolved in DMF (5 mL). BnBr (0.40 mL, 3.32 mmol, 1.2 eq) and K₂CO₃ (0.57 g, 4.16 mmol, 1.5 eq) were added. The suspension was stirred at rt for overnight. The reaction mixture was diluted with EtOAc, washed with water (50 mL×2) and brine (30 mL×1). The organic phase was concentrated under reduced pressure and the residue was purified on silica gel with 0 to 3% EtOAc/hexane to yield the desired product S13-3: MS (ESI) m/z 589.0 (M−H).

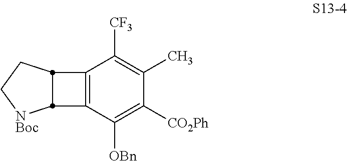

To compound S13-3 (632 mg, 1.07 mmol, 1 eq) in THF (5 mL) cooled at −78° C. was added iPrMgCl-LiCl (1.07 mL, 1.3 M/THF, 1.39 mmol, 1.3 eq) dropwise while maintaining reaction internal temperature between −72 to −75° C. The reaction was stirred at −78° C. for 30 min. 1-N-Boc-2,3-dihydropyrrole (0.92 mL, 5.33 mmol, 5 eq) was added dropwise. The reaction was gradually warmed up from −78° C. to rt over 2 h with stirring. The reaction was further stirred at rt for 48 hrs. EtOAc (100 mL) was added. The reaction mixture was washed with saturated aqueous ammonium chloride (50 mL×2) and brine (50 mL×1), dried over magnesium sulfate, and concentrated under reduced pressure. Column chromatography on silica gel with 0 to 8% EtOAc/hexane yielded the desired product S13-4 as a pale oil (224 mg, 38%): MS (ESI) m/z 576.4 (M+Na).

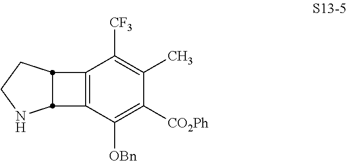

Compound S13-4 (224 mg, 0.40 mmol) was treated with 4 N HCl in dioxane at rt for 1 h. Saturated aqueous sodium bicarbonate (50 mL) was added and the reaction mixture was extracted with EtOAc (50 mL×3). The combined EtOAc extracts were dried over magnesium sulfate and concentrated under reduced pressure to give compound S13-5 as a pale solid (165 mg, 90%): ¹H NMR (400 MHz, CDCl₃) S7.20-7.60 (m, 8H), 7.10 (d, J=7.3 Hz, 2H), 5.56 (ABq, J=12.2, 28.1 Hz, 2H), 4.76 (d, J=3.6 Hz, 1H), 4.03 (br d, J=8.0 Hz, 1H), 3.10-3.20 (m, 1H), 2.65-2.80 (m, 1H), 2.49 (s, 3H), 1.90-2.00 (m, 1H), 1.55-1.70 (m, 1H); MS (ESI) m/z 454.4 (M+H).

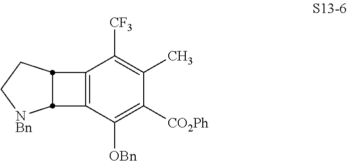

To compound S13-5 (165 mg, 0.36 mmol, 1 eq) in 1,2-dichloroethane (4 mL) was added HOAc (0.033 mL, 0.55 mmol, 1.5 eq), benzaldehyde (0.055 mL, 0.54 mmol, 1.5 eq), and Na(OAc)₃BH (116 mg, 0.55 mmol, 1.5 eq) at rt. The reaction mixture was stirred at rt for overnight, added with aqueous sodium bicarbonate (50 mL), and extracted with EtOAc (50 mL×3). The combined EtOAc extracts were dried over sodium sulfate and concentrated under reduced pressure. Column chromatography on silica gel with 0-8% EtOAc/hexane yielded the desired product S13-6 as a pale oil (178 mg, 91%): ¹H NMR (400 MHz, CDCl₃) S7.20-7.70 (m, 13H), 7.07 (d, J=7.4 Hz), 5.48 (br d, J=12.2 Hz, 1H), 5.18 (br d, J=12.2 Hz, 1H), 4.62 (br s, 1H), 4.23 (br s, 1H), 3.85 (br s, 1H), 3.64 (d, J=12.8 Hz, 1H), 3.01 (br s, 1H), 2.82 (br s, 1H), 2.49 (s, 3H), 2.04 (br s, 1H), 1.87 (br s, 1H); MS (ESI) m/z 544.4 (M+H).

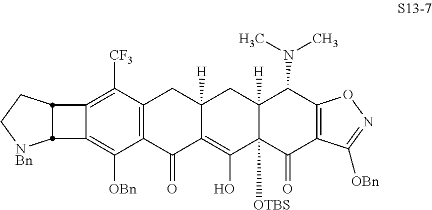 [SMILES: CN(C)[C@@H]1c2onc(OCc3ccccc3)c2C(=O)[C@@]2(O[Si](C)(C)C(C)(C)C)C(O)=C3C(=O)c4c(c(C(F)(F)F)c5c(c4OCc4ccccc4)[C@H]4[C@@H]5CCN4Cc4ccccc4)C[C@H]3C[C@@H]12]

To diisopropylamine (0.058 mL, 0.41 mmol, 1.25 eq) in THF (2 mL) at −78° C. was added nBuLi (0.164 mL, 2.5 M/hexane, 0.41 mmol, 1.25 eq) dropwise. The reaction was stirred at 0° C. for 10 min and cooled to −78° C. Compound S13-6 (178 mg, 0.33 mmol, in 4 mL THF) was added dropwise while maintaining the reaction internal temperature between −70 to −78° C. The resulting deep red solution was stirred at −78° C. for 30 min. LHMDS (0.41 mL, 1 M/THF, 0.41 mmol, 1.25 eq) and enone S5-5 (198 mg, 0.41 mmol, in 2 mL THF) were added dropwise while maintaining the reaction internal temperature between −70 to −78° C. The reaction was gradually warmed up from −78 to 0° C. over 2 h with stirring. Saturated aqueous sodium bicarbonate (50 mL) was added. The reaction mixture was extracted with EtOAc (50 mL×3). The combined EtOAc extracts were dried over magnesium sulfate. Column chromatography on silica gel with 0 to 25% EtOAc/hexane yielded the two diastereomers of desired product as yellow foams. S13-7A, diastereomer A (125 mg, 41%): ¹H NMR (400 MHz, CDCl₃) δ 16.01 (s, 1H), 7.18-7.50 (m, 11H), 6.80-6.90 (m, 4H), 5.49 (br s, 2H), 5.36 (s, 2 H), 4.97 (s, 2H), 4.50 (br s, 1H), 4.13 (br s, 1H), 3.94 (d, J=13.0 Hz, 1H), 3.76 (br s, 1H), 3.62 (d, J=13.4 Hz, 1H), 3.19 (br d, J=16.5 Hz, 1H), 2.90-3.05 (m, 2H), 2.40-3.80 (m, 4 H), 2.48 (s, 6H), 2.11 (br d, J=14.7 Hz, 1H), 0.85 (s, 9H), 0.28 (s, 3H), 0.16 (s, 3H); MS (ESI) m/z 932.6 (M+H). S13-7B, diastereomer B (136 mg, 44%): ¹H NMR (400 MHz, CDCl₃) δ 15.87 (s, 1H), 6.85-7.45 (m, 15H), 6.05 (d, J=10.4 Hz, 1H), 5.35 (br s, 1H), 5.25-5.35 (m, 1H), 5.30 (d, J=10.2 Hz, 2H), 4.51 (br s, 1H), 4.07 (br s, 1H), 3.90 (d, J=13.1 Hz, 1H), 3.70-3.80 (m, 1H), 3.75 (d, J=13.0 Hz, 1H), 3.55-3.65 (m, 1H), 3.08-3.18 (m, 1H), 2.00-2.95 (m, 6H), 2.40 (s, 6H), 0.80 (s, 9H), 0.00-0.25 (m, 6H); MS (ESI) m/z 932.6 (M+H).

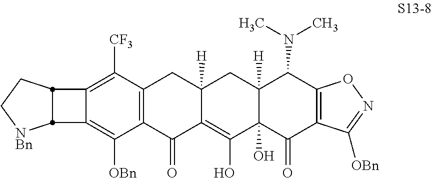 [SMILES: CN(C)[C@@H]1c2onc(OCc3ccccc3)c2C(=O)[C@@]2(O)C(O)=C3C(=O)c4c(c(C(F)(F)F)c5c(c4OCc4ccccc4)[C@H]4[C@@H]5CCN4Cc4ccccc4)C[C@H]3C[C@@H]12]

Compound S13-7A (125 mg, 0.134 mmol) in dioxane (4 mL) was treated with 48% aqueous HF (4 mL) at rt for overnight. The reaction mixture was slowly added into a vigorously stirred saturated aqueous K₂HPO₄ solution (160 mL). The mixture was extracted with EtOAc (50 mL×3). The EtOAc extracts were combined, dried over magnesium sulfate, and concentrated under reduced pressure to yield the crude product S13-8A as a yellow foam: MS (ESI) m/z 818.5 (M+H). Similarly, compound S13-7B (136 mg, 0.146 mmol) was desilylated to give compound S13-8B as a yellow foam: MS (ESI) m/z 818.5 (M+H).

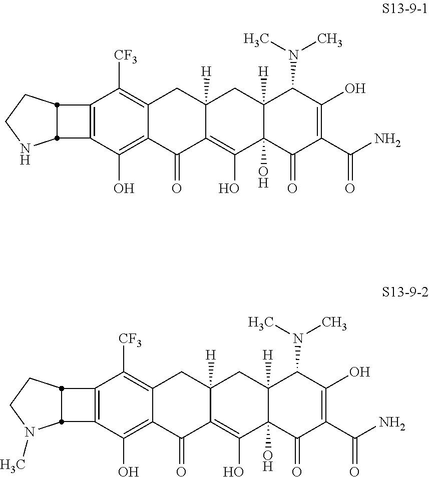 [SMILES: CN(C)[C@@H]1C(O)=C(C(N)=O)C(=O)[C@@]2(O)C(O)=C3C(=O)c4c(O)c5c(c(C(F)(F)F)c4C[C@H]3C[C@@H]12)[C@H]1CCN[C@@H]51 ; CN(C)[C@@H]1C(O)=C(C(N)=O)C(=O)[C@@]2(O)C(O)=C3C(=O)c4c(O)c5c(c(C(F)(F)F)c4C[C@H]3C[C@@H]12)[C@H]1CCN(C)[C@@H]51]

Compound S13-8A (0.134 mmol, crude) was dissolved in dioxane:methanol (3:1, v/v, 4 mL). HCl (0.5 M/aqueous methanol, 1 mL) and 10% Pd—C (29 mg, 0.014 mmol, 0.1 eq) were added. The reaction mixture was then stirred under H₂ (1 atm) for 4 hrs. Half of the reaction mixture (2.5 mL) was removed from the reaction vessel and filtered through a small Celite pad. The Celite pad was washed with methanol (2 mL×3). The combined filtrates were concentrated under reduced pressure. The crude product was purified by preparative HPLC with a gradient of 5% acetonitrile/0.05 N HCl to 40% acetonitrile/0.05 N HCl over 20 min to yield the desired product S13-9-1A as a yellow solid after lyophilization (22 mg, bis-HCl salt, 53%): ¹H NMR (400 MHz, CD₃OD) δ 5.31 (d, J=3.7 Hz, 1H), 4.40 (br d, J=5.5 Hz, 1H), 4.13 (s, 1H), 3.64 (dd, J=6.7, 11.6 Hz, 1H), 2.90-3.20 (m, 4H), 3.05 (s, 3H), 2.95 (s, 3H), 2.50-2.62 (m, 1H), 2.10-2.30 (m 3 H), 1.55-1.70 (m, 1H); MS (ESI) m/z 550.4 (M+H).

One half of the above reaction mixture (2.5 mL) was added with formaldehyde (0.10 mL, 37% in water, 1.33 mmol, 20 eq). The reaction mixture was stirred under H₂ (1 atm) at rt for 72 h and filtered through a small Celite pad. The Celite pad was washed with methanol (2 mL×3) and the combined filtrates were concentrated under reduced pressure. The crude product was purified by preparative HPLC with a gradient of 5% acetonitrile/0.05 N HCl to 40% acetonitrile/0.05 N HCl over 20 min to yield the desired product S13-9-2A as an orange solid after lyophilization (16 mg, bis-HCl salt, 38%): ¹H NMR (400 MHz, CD₃OD) δ 5.42 (d, J=3.0 Hz, 1H), 4.40 (br s, 1H), 4.13 (s, 1H), 3.70-3.80 (m, 1H), 2.94-3.15 (m, 4H), 3.08 (s, 3H), 3.05 (s, 3H), 2.95 (s, 3H), 2.55-2.65 (m, 1H), 2.20-2.35 (m, 3H), 1.58-1.70 (m, 1 H); MS (ESI) m/z 564.3 (M+H).

Compound S13-8B (0.146 mmol, crude) was similarly treated as S13-8A to yield the following desired compounds: S13-9-1B (19 mg, bis-HCl salt, yellow solid, 42%): ¹H NMR (400 MHz, CD₃OD) δ 5.30 (d, J=3.0 Hz, 1H), 4.40 (br d, J=5.5 Hz, 1H), 4.13 (s, 1H), 3.63 (dd, J=6.3, 11.6 Hz, 1H), 2.90-3.22 (m, 4H), 3.04 (s, 3H), 2.94 (s, 3H), 2.52-2.61 (m, 1H), 2.08-2.30 (m, 3H), 1.56-1.68 (m, 1H); MS (ESI) m/z 550.4 (M+H).

S13-9-2B (18 mg, bis-HCl salt, yellow solid, 39%): ¹H NMR (400 MHz, CD₃OD) S 5.41 (d, J=2.8 Hz, 1H), 4.39 (br s, 1H), 4.14 (s, 1H), 3.70-3.78 (m, 1H), 2.90-3.25 (m, 4 H), 3.11 (s, 3H), 3.04 (s, 3H), 2.95 (s, 3H), 2.54-2.63 (m, 1H), 2.20-2.35 (m, 3H), 1.58-1.69 (m, 1H); MS (ESI) m/z 564.3 (M+H).

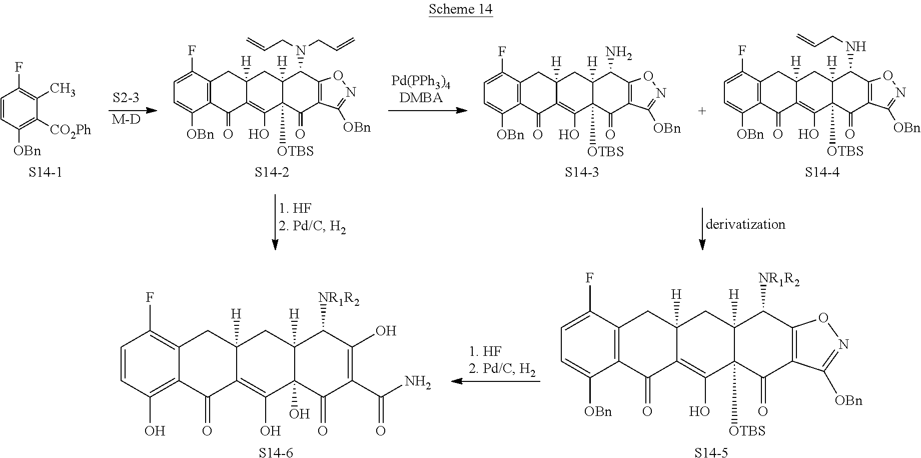

The following compounds were prepared per Scheme 14.

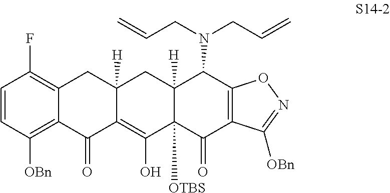 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)ccc(F)c4C[C@H]3C[C@@H]12]

Compound S14-2 was prepared from compound S14-1 (obtained via standard benzylation of the corresponding phenol, which was prepared according to literature procedures including WO2012/021712 A1) and diallylenone S2-3 by using General Procedure E: ¹H NMR (400 MHz, CDCl₃) δ 16.05 (s, 1H), 7.52-7.42 (m, 4H), 7.41-7.25 (m, 6H), 7.13-7.07 (m, 1H), 6.83 (dd, J=9.4, 4.1 Hz, 1H), 5.85-5.73 (m, 2H), 5.36 (s, 2H), 5.24-5.07 (m, 6H), 4.08 (d, J=10.1 Hz, 1H), 3.36-3.27 (m, 2H), 3.25-3.10 (m, 3H), 3.04-2.9 (m, 1H), 2.68-2.57 (m, 1H), 2.54-2.39 (m, 2H), 2.15-2.08 (m, 1H), 0.816 (s, 9H), 0.25 (s, 3H), 0.12 (s, 3H); MS (ESI) m/z 777.58 (M+H).

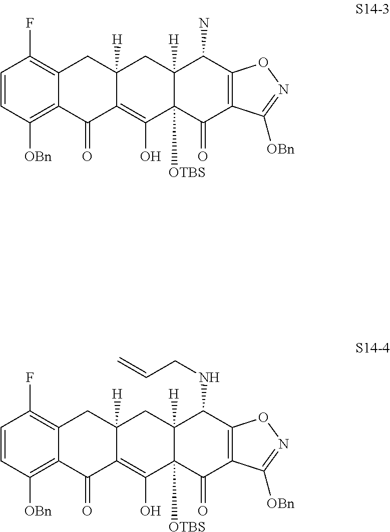 [SMILES: CC(C)(C)[Si](C)(C)O[C@@]12C(=O)c3c(OCc4ccccc4)noc3[C@@H](N)[C@@H]1C[C@@H]1Cc3c(F)ccc(OCc4ccccc4)c3C(=O)C1=C2O ; C=CCN[C@@H]1c2onc(OCc3ccccc3)c2C(=O)[C@@]2(O[Si](C)(C)C(C)(C)C)C(O)=C3C(=O)c4c(OCc5ccccc5)ccc(F)c4C[C@H]3C[C@@H]12]

Compound S14-3 and S14-4 were prepared from compound S14-2 by using General Procedure A. S14-3: ¹H NMR (400 MHz, CDCl₃) δ 16.61 (s, 1H), 7.54-7.42 (m, 4H), 7.42-7.26 (m, 6H), 7.08 (t, J=8.4 Hz, 1H), 6.83 (dd, J=9.0, 4.0 Hz, 1H), 5.39, 5.35 (ABq, J=12.2 Hz, 2H), 5.23, 5.14 (ABq, J=12.2 Hz, 2H), 3.92 (d, J=2.4 Hz, 1H), 3.02 (dd, J=16.0, 3.6 Hz, 1H), 2.87-2.75 (m, 1H), 2.64-2.57 (m, 1H), 2.19 (t, J=16.0 Hz, 1H), 2.15-2.05 (m, 2H), 0.73 (s, 9H), 0.20 (s, 3H), 0.09 (s, 3H); MS (ESI) 697.53 m/z (M+H). S14-4: ¹H NMR (400 MHz, CDCl₃) δ 516.66 (s, 1H), 7.54-7.42 (m, 4H), 7.42-7.25 (m, 6H), 7.10-7.04 (m, 1H), 6.83 (dd, J=9.2, 4.5 Hz, 1H), 5.93-5.78 (m, 1H), 5.41-5.34 (m, 2H), 5.30-5.08 (m, 4H), 4.69 (d, J=6.1 Hz, 1H), 3.76-3.70 (m, 1H), 3.58-3.50 (m, 1H), 3.46-3.37 (m, 1H), 3.02-2.94 (m, 1H), 2.83-2.67 (m, 2H), 2.15 (t, J=15.0 Hz, 1H), 2.06-1.98 (m, 1H), 0.72 (s, 9H), 0.20 (s, 3H), 0.07 (s, 3H); MS (ESI) m/z 737.51 (M+H).

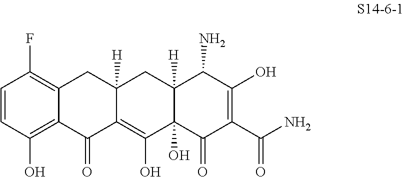 [SMILES: NC(=O)C1=C(O)[C@@H](N)[C@@H]2C[C@@H]3Cc4c(F)ccc(O)c4C(=O)C3=C(O)[C@]2(O)C1=O]

Compound S14-6-1 was prepared from compound S14-3 by using General Procedures C and D-2: S14-6-1: ¹H NMR (400 MHz, CD₃OD, hydrochloride salt) δ 7.26 (t, J=8.9 Hz, 1H), 6.80 (dd, J=9.2 4.0 Hz, 1H), 3.87 (s, 1H), 3.15 (dd, J=15.3, 4.9 Hz, 1H), 2.97 (qd, J=9.8, 4.9 Hz, 1H), 2.61 (dt, J=12.6, 2.1 Hz, 1H), 2.29 (t, J=10.4 Hz, 1H), (qd, J=13.7, 2.4 Hz, 1H), 1.59 (td, J=13.3, 10.6 Hz, 1H); MS (ESI) m/z 405.25 (M+H).

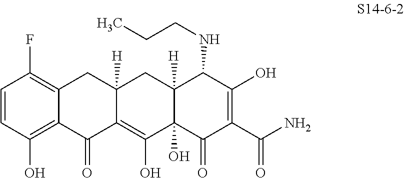 [SMILES: CCCN[C@@H]1C(O)=C(C(N)=O)C(=O)[C@@]2(O)C(O)=C3C(=O)c4c(O)ccc(F)c4C[C@H]3C[C@@H]12]

Compound S14-6-2 was prepared from compound S14-2 by using General Procedures C and D-2: S14-6-2 ¹H NMR (400 MHz, CD₃OD, hydrochloride salt) δ 7.26 (t, J=9.2 Hz, 1H), 6.81 (dd, J=9.2, 4.0 Hz, 1H), 3.86 (s, 1H), 3.27-3.17 (m, 2H), 3.16-3.09 (m, 1H), 3.04-2.92 (m, 1H), 2.82 (d, J=12.8 Hz, 1H), 2.27 (t, J=14.6 Hz, 1H), 2.19 (dq, J=13.6, 2.6 Hz, 1H), 1.76 (td, J=15.6, 7.7 Hz, 2H), 1.57 (td, J=13.4, 11.0 Hz, 1H), 1.03 (t, J=7.3 Hz, 3H); MS (ESI) m/z 447.33 (M+H).

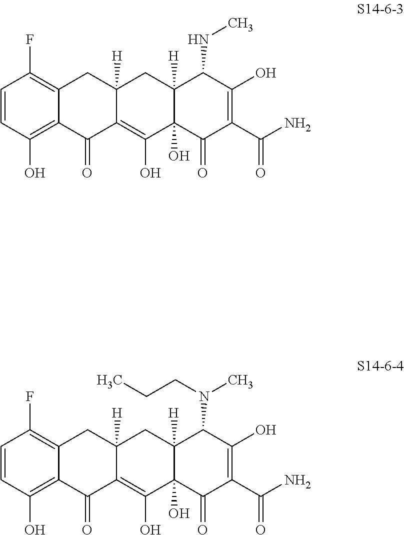 [SMILES: CN[C@@H]1C(O)=C(C(N)=O)C(=O)[C@@]2(O)C(O)=C3C(=O)c4c(O)ccc(F)c4C[C@H]3C[C@@H]12 ; CCCN(C)[C@@H]1C(O)=C(C(N)=O)C(=O)[C@@]2(O)C(O)=C3C(=O)c4c(O)ccc(F)c4C[C@H]3C[C@@H]12]

Compounds S14-6-3 and S14-6-4 were prepared from compound S14-4 with HCHO by using General Procedures B-1, C, and D2. S14-6-3: ¹H NMR (400 MHz, CD₃OD, hydrochloride salt) δ 7.27 (t, J=8.9 Hz, 1H), 6.81 (dd, J=9.2, 4.0 Hz, 1H), 3.78 (s, 1H), 3.14 (dd, J=15.0, 4.6 Hz, 1H), 3.04-2.93 (m, 2H), 2.90 (s, 3H), 2.80-2.73 (m, 1H), 2.28 (t, J=14.6 Hz, 1H), 2.18 (dq, J=13.6, 2.6 Hz, 1H), 1.62-1.50 (m, 1H), MS (ESI) m/z 419.32 (M+H). S14-6-4: ¹H NMR (400 MHz, CD₃OD, hydrochloride salt) δ 7.27 (t, J=9.2 Hz, 1H), 6.81 (dd, J=9.2, 4.0 Hz, 1H), 4.19 (s, 0.5H), 4.09 (s, 0.5H), 3.39-3.31 (m, 1H) 3.22-3.09 (m, 2H), 3.08-2.86 (m, 5H), 2.34-2.13 (m, 2H), 1.90-1.56 (m, 3H), 1.08-0.95 (m, 3H); MS (ESI) m/z 461.32 (M+H).

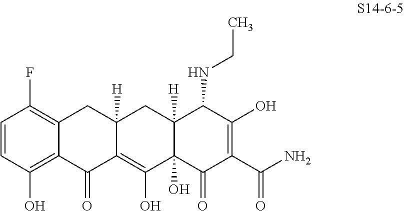 [SMILES: CCN[C@@H]1C(O)=C(C(N)=O)C(=O)[C@@]2(O)C(O)=C3C(=O)c4c(O)ccc(F)c4C[C@H]3C[C@@H]12]

Compound S14-6-5 was prepared from compound S14-3 with CH₃CHO by using General Procedures B-1 (at 0° C.), C, and D2: ¹H NMR (400 MHz, CD₃OD, hydrochloride salt) δ 7.26 (t, J=8.9 Hz, 1H), 6.81 (dd, J=9.2, 4.0 Hz, 1H), 3.84 (s, 1H), 3.48-3.30 (m, 2H), 3.14 (dd, J=14.6, 4.3 Hz, 1H), 3.03-2.92 (m, 1H), 2.79 (d, J=12.2 Hz, 1H), 2.27 (t, J=14.4 Hz, 1H), 2.19 (qd, J=11.2, 3.2 Hz, 1H), 1.62-1.50 (m, 1H), 1.35 (t, J=7.3 Hz, 3H); MS (ESI) m/z 433.31 (M+H).

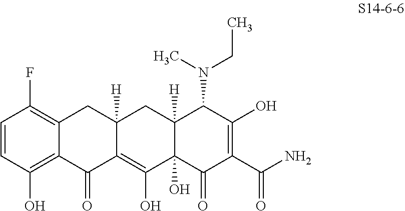 [SMILES: CCN(C)[C@@H]1C(O)=C(C(N)=O)C(=O)[C@@]2(O)C(O)=C3C(=O)c4c(O)ccc(F)c4C[C@H]3C[C@@H]12]

Compound S14-6-6 was prepared from compound S14-3 with CH₃CHO by using General Procedures B-1 (at 0° C.), then B-1 again with HCHO, C and D-2: ¹H NMR (400 MHz, CD₃OD, hydrochloride salt) δ 7.27 (t, J=8.9 Hz, 1H), 6.81 (dd, J=9.2, 4.0 Hz, 1H), 4.21 (s, 0.5H). 4.10 (s, 0.5H), 3.52-3.41 (m, 1H), 3.38-3.29 (m, 1h), 3.19-3.11 (m, 1H), 3.09-2.85 (m, 5H), 2.34-2.15 (m, 2H), 1.71-1.56 (m, 1H), 1.44-1.33 (m, 3H); MS (ESI) m/z 447.29 (M+H).

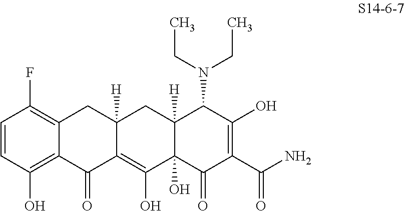 [SMILES: CCN(CC)[C@@H]1C(O)=C(C(N)=O)C(=O)[C@@]2(O)C(O)=C3C(=O)c4c(O)ccc(F)c4C[C@H]3C[C@@H]12]

Compound S14-6-7 was prepared from compound S14-3 with CH₃CHO by using General Procedures B-1, C, and D2: ¹H NMR (400 MHz, CD₃OD, hydrochloride salt) δ 7.27 (t, J=9.2 Hz, 1H), 6.81 (dd, J=9.2, 4.0 Hz, 1H), 4.23 (s, 1H), 3.63-3.52 (m, 1H), 3.80-3.40 (m, 2H), 3.35-3.24 (m, 1H), 3.19-3.11 (m, 1H), 3.07-2.96 (m, 1H), 2.88 (d, J=12.8 Hz, 1H), 2.32-2.16 (m, 2), 1.69-1.56 (m, 1H), 1.40 (t, J=7.0 Hz, 6H); MS (ESI) m/z 461.32 (M+H).

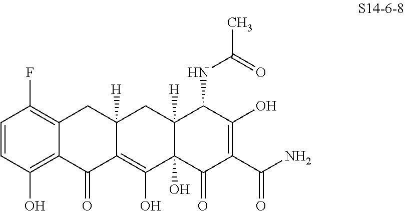 [SMILES: CC(=O)N[C@@H]1C(O)=C(C(N)=O)C(=O)[C@@]2(O)C(O)=C3C(=O)c4c(O)ccc(F)c4C[C@H]3C[C@@H]12]

Compound S14-6-8 was prepared from compound S14-3 with Ac₂O using General Procedures B-2, C, and D-2: ¹H NMR (400 MHz, CD₃OD, hydrochloride salt) δ 7.23 (t, J=9.2 Hz, 1H), 6.76 (dd, J=9.2, 3.7 Hz, 1H), 4.70-4.59 (m, 1H), 3.10-3.03 (m, 1H), 3.02-2.91 (m, 1H), 2.53-2.30 (m, 2H), 2.03 (s, 3H), 1.65-1.56 (m, 1H); MS (ESI) m/z 447.24 (M+H).

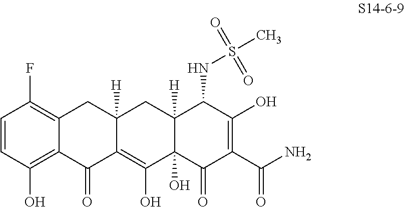 [SMILES: CS(=O)(=O)N[C@@H]1C(O)=C(C(N)=O)C(=O)[C@@]2(O)C(O)=C3C(=O)c4c(O)ccc(F)c4C[C@H]3C[C@@H]12]

Compound S14-6-9 was prepared from compound S14-3 with Ms₂O using General Procedures B-2, C, and D-2: ¹H NMR (400 MHz, CD₃OD, hydrochloride salt) S7.24 (t, J=8.9 Hz, 1H), 6.77 (dd, J=8.9, 4.0 Hz, 1H), 4.09 (d, J=4.3 Hz, 1H), 3.16-3.08 (m, 4H), 3.04-2.92 (m, 1H), 2.53-2.40 (m, 2H), 2.31-2.23 (m, 1H), 1.72-1.61 (m, 1H); MS (ESI) m/z 483.1 (M+H).

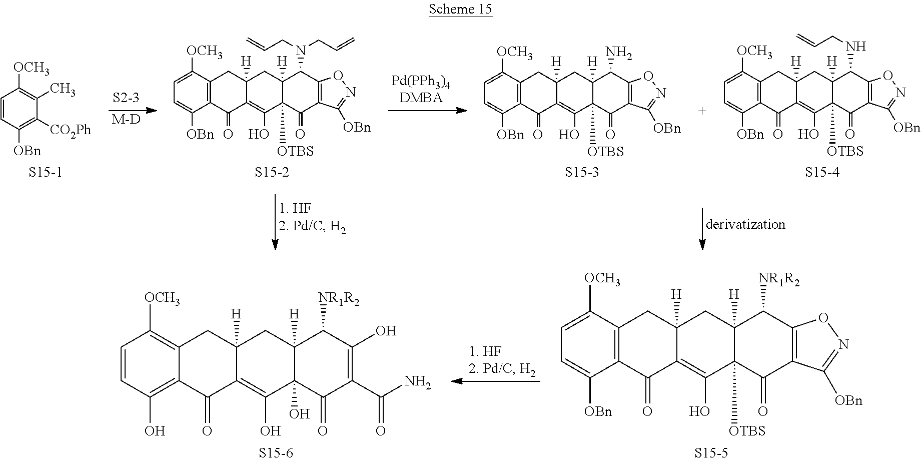

The following compounds were prepared per Scheme 15.

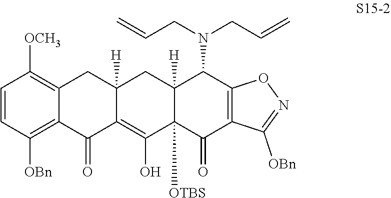 [SMILES: C=CCN(CC=C)[C@@H]1c2onc(OCc3ccccc3)c2C(=O)[C@@]2(O[Si](C)(C)C(C)(C)C)C(O)=C3C(=O)c4c(OCc5ccccc5)ccc(OC)c4C[C@H]3C[C@@H]12]

Compound S15-2 was prepared from compound S15-1 (prepared according to literature procedures including WO2011/025982 A2) and diallylenone S2-3 by using General Procedure E: ¹H NMR (400 MHz, CDCl₃) δ 16.07 (s, 1H), 7.51-7.43 (m, 4H), 7.40-7.25 (m, 6H), 6.92, 6.82 (ABq, J=8.8 Hz, 2H), 5.88-5.73 (m, 2H), 5.35 (s, 2H), 5.23-5.06 (m, 6H), 4.11 (d, J=9.8 Hz, 1H), 3.80 (s, 3H), 3.36-3.15 (m, 5H), 3.00-2.77 (m, 1H), 2.56-2.34 (m, 3H), 2.15-2.08 (m, 1H), 0.81 (s, 9H), 0.25 (s, 3H), 0.12 (s, 3H); MS (ESI) m/z 789.55 (M+H).

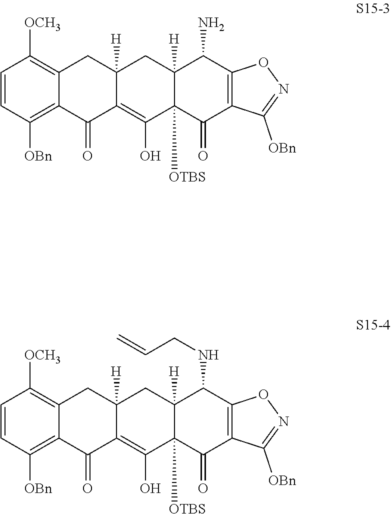 [SMILES: COc1ccc(OCc2ccccc2)c2c1C[C@H]1C[C@H]3[C@H](N)c4onc(OCc5ccccc5)c4C(=O)[C@@]3(O[Si](C)(C)C(C)(C)C)C(O)=C1C2=O ; C=CCN[C@@H]1c2onc(OCc3ccccc3)c2C(=O)[C@@]2(O[Si](C)(C)C(C)(C)C)C(O)=C3C(=O)c4c(OCc5ccccc5)ccc(OC)c4C[C@H]3C[C@@H]12]

Compound S15-3 and S15-4 were prepared from compound S15-2 by using General Procedure A. S15-3: ¹H NMR (400 MHz, CDCl₃) δ 16.63 (s, 1H), 7.53-7.46 (m, 4H), 7.41-7.27 (m, 6H), 6.93 (d, J=9.2 Hz, 1H), 6.85 (d, J=9.2 Hz, 1H), 5.41, 5.36 (ABq, J=12.1 Hz, 2H), 5.22, 5.12 (ABq, J=12.1 Hz, 2H), 3.96-3.92 (m, 1H), 3.66 (s, 3H), 3.16 (dd, J=15.9, 4.3 Hz, 1H), 2.84-2.72 (m, 1H), 2.64-2.57 (m, 1H), 2.13-2.06 (m, 3H), 0.75 (s, 9H), 0.22 (s, 3H), 0.12 (s, 3H); MS (ESI) m/z 709.49 (M+H). S15-3: ¹H NMR (400 MHz, CDCl₃) δ 16.70 (s, 1H), 7.54-7.46 (m, 4H), 7.41-7.28 (m, 6H), 6.93 (d, J=9.2, 1H), 6.85 (d, J=9.2 Hz, 1H), 5.95-5.84 (m, 1H), 5.42, 5.37 (ABq, J=12.2 Hz, 2H), 5.32-5.08 (m, 4H), 3.77 (s, 3H), 3.56 (dd, J=13.2, 6.7 Hz, 1H), 3.47-3.39 (m, 1H), 3.11 (dd, J=15.9, 4.9 Hz, 1H), 2.80-2.68 (m, 2H), 2.61-2.45 (m, 1H), 2.08-1.98 (m, 2H), 1.51-1.39 (m, 1H), 0.73 (s, 9H), 0.22 (s, 3H), 0.10 (s, 3H); MS (ESI) m/z 749.48 (M+H).

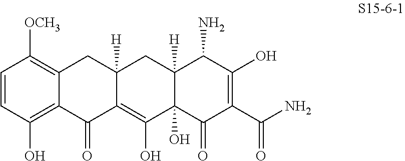 [SMILES: COc1ccc(O)c2c1C[C@H]1C[C@H]3[C@H](N)C(O)=C(C(N)=O)C(=O)[C@@]3(O)C(O)=C1C2=O]

Compound S15-6-1 was prepared from compound S15-3 by using General Procedures C and D-2: S15-6-1: ¹H NMR (400 MHz, CD₃OD, hydrochloride salt) δ 7.21 (d, J=9.2 Hz, 1H), 6.78 (d, J=9.2 Hz, 1H), 3.83 (s, 1H), 3.77 (s, 3H), 2.93-2.82 (m, 1H), 2.60-2.52 (m, 1H), 2.22-2.07 (m, 2H), 1.63-1.50 (m, 1H); MS (ESI) m/z 417.25 (M+H).

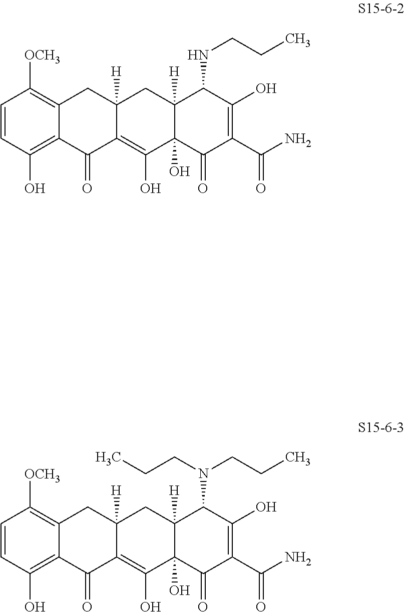 [SMILES: CCCN[C@@H]1C(O)=C(C(N)=O)C(=O)[C@@]2(O)C(O)=C3C(=O)c4c(O)ccc(OC)c4C[C@H]3C[C@@H]12 ; CCCN(CCC)[C@@H]1C(O)=C(C(N)=O)C(=O)[C@@]2(O)C(O)=C3C(=O)c4c(O)ccc(OC)c4C[C@H]3C[C@@H]12]

Compounds S15-6-2 and S15-6-3 were prepared from compound S15-2 by using General Procedures C and D-2. S15-6-2: ¹H NMR (400 MHz, CD₃OD, hydrochloride salt) S 7.21 (d, J=9.2 Hz, 1H), 6.78 (d, J=9.2 Hz, 1H), 3.85 (s, 1H), 3.77 (s, 3H), 3.28-3.14 (m, 3H), 2.96-2.84 (m, 1H), 2.80 (d, J=12.2 Hz, 1H), 2.20-2.05 (m, 2H), 1.81-1.65 (m, 2H), 1.60-1.48 (m, 1H), 1.02 (t. J=7.3 Hz, 3H); MS (ESI) m/z 459.4 (M+H). S15-6-3: ¹H NMR (400 MHz, CD₃OD, hydrochloride salt) δ 7.22 (d, J=9.2 Hz, 1H), 6.78 (d, J=9.2 Hz, 1H), 4.18 (s, 1H), 3.77 (s, 3H), 3.39-3.14 (m, 5H), 3.04-2.64 (m, 2H), 2.20-2.08 (m, 2H), 1.90-1.74 (m, 2H), 1.70-1.52 (m, 1H), 1.08-0.98 (m, 6H); MS (ESI) m/z 501.3 (M+H).

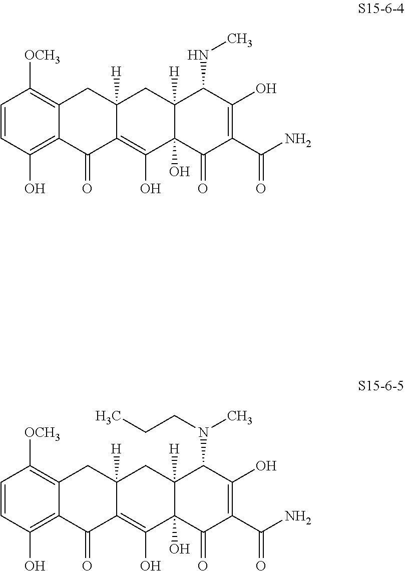 [SMILES: CN[C@@H]1C(O)=C(C(N)=O)C(=O)[C@@]2(O)C(O)=C3C(=O)c4c(O)ccc(OC)c4C[C@H]3C[C@@H]12 ; CCCN(C)[C@@H]1C(O)=C(C(N)=O)C(=O)[C@@]2(O)C(O)=C3C(=O)c4c(O)ccc(OC)c4C[C@H]3C[C@@H]12]

Compounds S14-6-4 and S14-6-5 were prepared from compound S15-4 with HCHO by using General Procedures B-1, C, and D2. S15-6-4: ¹H NMR (400 MHz, CD₃OD, hydrochloride salt) δ 7.21 (d, J=9.2 Hz, 1H), 6.78 (d, J=9.2 Hz, 1H), 3.80-3.76 (m, 4H), 3.26-3.20 (m, 1H), 2.95-2.84 (m, 4H), 2.78-2.71 (m, 1H), 2.19-2.04 (m, 2H), 1.60-1.47 (m, 1H); MS (ESI) m/z 431.2 (M+H). S15-6-5: ¹H NMR (400 MHz, CD₃OD, hydrochloride salt, rotamers) δ 7.21 (d, J=9.2 Hz, 1H), 6.78 (d, J=9.2 Hz, 1H), 4.18 (s, 0.5H), 4.08 (s, 0.5H), 3.78 (s, 3H), 3.40-3.23 (m, 3H), 3.22-3.10 (m, 1H), 3.04-2.85 (m, 4H), 2.23-2.06 (m, 2H), 1.90-1.69 (m, 2H), 1.69-1.54 (m, 1H), 1.07-0.96 (m, 3H); MS (ESI) m/z 473.2 (M+H).

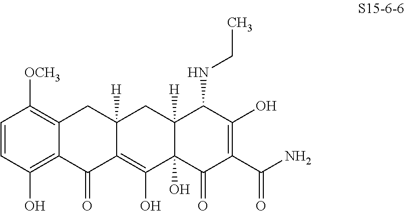 [SMILES: CCN[C@@H]1C(O)=C(C(N)=O)C(=O)[C@@]2(O)C(O)=C3C(=O)c4c(O)ccc(OC)c4C[C@H]3C[C@@H]12]

Compound S15-6-6 was prepared from compound S15-3 with CH₃CHO by using General Procedures B-1 (at 0° C.), C, and D2: ¹H NMR (400 MHz, CD₃OD, hydrochloride salt) δ 7.21 (d, J=9.2 Hz, 1H), 6.77 (d, J=9.2 Hz, 1H), 3.84 (s, 1H), 3.77 (s, 3H), 3.45-3.20 (m, 2H), 2.96-2.83 (m. 1H), 2.78 (d, J=12.8 Hz, 1H), 2.21-2.00 (m, 2H), 1.59-1.46 (m, 1H), 1.35 (t, J=7.3 Hz, 3H); MS (ESI) m/z 445.2 (M+H).

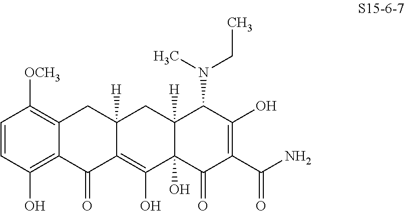 [SMILES: CCN(C)[C@@H]1C(O)=C(C(N)=O)C(=O)[C@@]2(O)C(O)=C3C(=O)c4c(O)ccc(OC)c4C[C@H]3C[C@@H]12]

Compound S15-6-7 was prepared from compound S15-3 with CH₃CHO by using General Procedures B-1 (at 0° C.), then B-1 again with HCHO, C and D-2: ¹H NMR (400 MHz, CD₃OD, hydrochloride salt, rotamers) δ 7.22 (d, J=9.2 Hz, 1H), 6.77 (d, J=9.2 Hz, 1H), 4.20 (s, 0.5H), 4.09 (s, 0.5H), 3.77 (s, 3H), 3.52-3.40 (m, 1H), 3.38-3.22 (m, 2H), 3.04-2.83 (m, 5H), 2.23-2.06 (m, 2H), 1.70-1.53 (m, 1H), 1.44-1.33 (m, 3H); MS (ESI) m/z 459.2 (M+H).

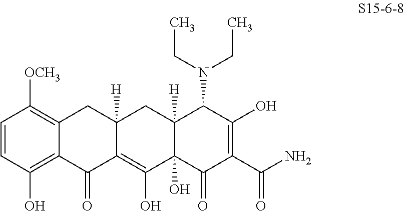 [SMILES: CCN(CC)[C@@H]1C(O)=C(C(N)=O)C(=O)[C@@]2(O)C(O)=C3C(=O)c4c(O)ccc(OC)c4C[C@H]3C[C@@H]12]

Compound S15-6-8 was prepared from compound S15-3 with CH₃CHO by using General Procedures B-1, C, and D2: ¹H NMR (400 MHz, CD₃OD, hydrochloride salt) δ 7.21 (d, J=9.2 Hz, 1H), 6.77 (d, J=9.2 Hz, 1H), 4.22 (s, 1H), 3.77 (s, 3H), 3.64-3.52 (m, 1H), 3.48-3.37 (m, 2H), 3.30-3.23 (m, 2H), 3.01-2.81 (m, 2H), 2.23-2.05 (m, 2H), 1.66-1.53 (m, 1H), 1.39 (t, J=7.3 Hz, 6H); MS (ESI) m/z 473.2 (M+H).

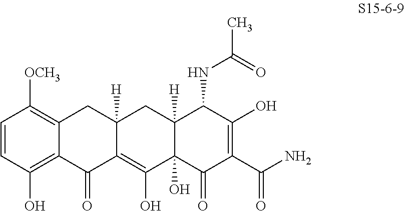 [SMILES: COc1ccc(O)c2c1C[C@H]1C[C@H]3[C@H](NC(C)=O)C(O)=C(C(N)=O)C(=O)[C@@]3(O)C(O)=C1C2=O]

Compound S15-6-9 was prepared from compound S15-3 with Ac₂O using General Procedures B-2, C, and D-2: ¹H NMR (400 MHz, CD₃OD, hydrochloride salt, rotamers) δ 7.18 (d, J=9.2 Hz, 1H), 6.75 (d, J=8.5 Hz, 1H), 4.71-4.64 (m, 1H), 3.77 (s, 3H), 3.20 (dd, J=16.5, 4.9 Hz, 1H), 2.94-2.84 (m, 1H), 2.46-2.22 (m, 3H), 2.03 (s, 3H), 1.63-1.52 (m, 1H); MS (ESI) m/z 459.2 (M+H).

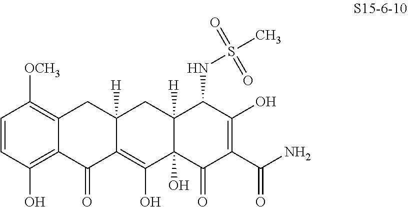 [SMILES: COc1ccc(O)c2c1C[C@H]1C[C@H]3[C@H](NS(C)(=O)=O)C(O)=C(C(N)=O)C(=O)[C@@]3(O)C(O)=C1C2=O]

Compound S15-6-10 was prepared from compound S15-3 with Ms₂O using General Procedures B-2, C, and D-2: ¹H NMR (400 MHz, CD₃OD, hydrochloride salt, rotamers) δ 7.18 (d, J=9.2 Hz, 1H), 6.74 (d, J=9.2 Hz, 1H), 4.71-4.64 (m, 1H), 4.08 (d, J=4.3 Hz, 1H), 3.77 (s, 3H), 3.23 (dd, J=15.9, 4.9 Hz, 1H), 3.13 (s, 3H), 2.95-2.84 (m, 1H), 2.48 (td, J=7.2, 3.5 Hz, 1H), 2.33-2.18 (m, 2H), 1.69-1.58 (m, 1H); MS (ESI) m/z 495.18 (M+H).

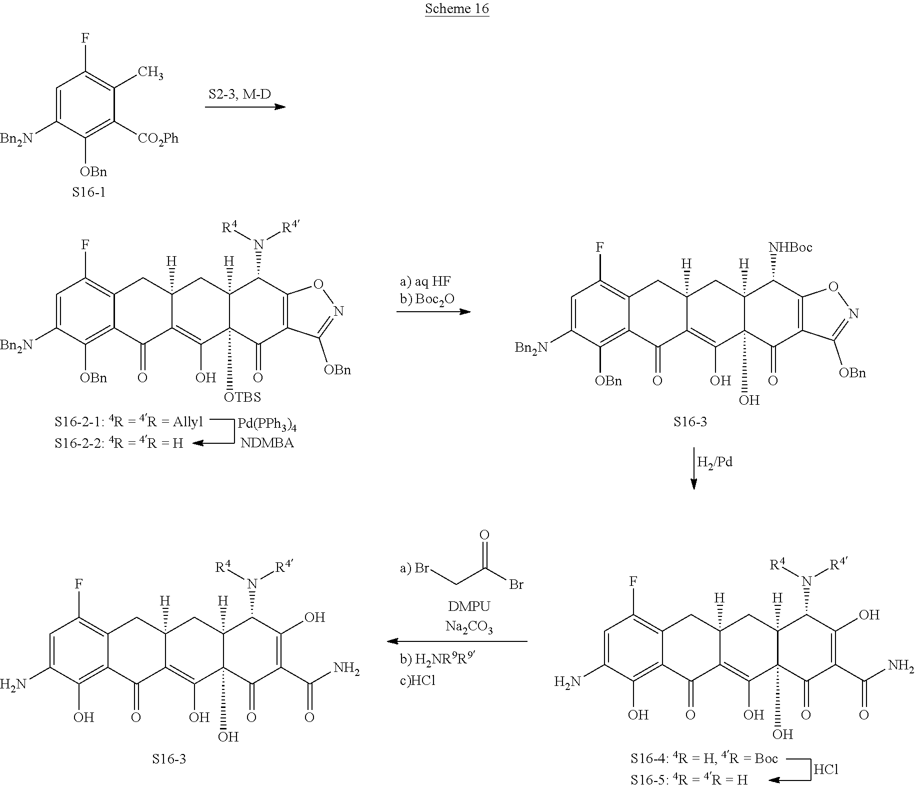

The following compounds were prepared per Scheme 16.

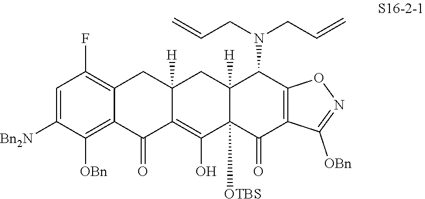

Compound S16-2-1 was prepared from S16-1 (6.574 g, 12.36 mmol, 2.1 eq) and C-4 ethylmethylamino enone S2-3 (3.149 g, 5.89 mmol, 1 eq) by using General Procedure E. Product S16-2-1 (1.321 g, 23%): ¹H NMR (400 MHz, CDCl₃) δ 16.17 (s, 1H), 7.55-7.50 (m, 4H), 7.41-7.30 (m, 8H), 7.29-7.22 (m, 4H), 7.18-7.11 (m, 4H), 6.68 (d, J=11.0 Hz, 1H), 5.88-5.76 (m, 2H), 5.37 (s, 2H), 5.27-5.10 (m, 5H), 5.00 (d, J=9.5 Hz, 1H), 4.33 (d, J=14.6 Hz, 2H), 4.19 (d, J=14.0 Hz, 2H), 3.38-3.19 (m, 4H), 3.13-2.95 (m, 2H), 2.17-2.10 (m, 1H), 0.83 (s, 9H), 0.26 (s, 3H), 0.15 (s, 3H); MS (ESI) m/z 972.55 (M+H).

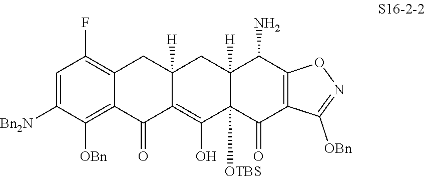

Compound S16-2-2 was prepared from compound S16-2-1 (1.321 g, 1.36 mmol, 1 eq) by using General Procedure A. S16-2-2 (884 mg, 72%): ¹H NMR (400 MHz, CDCl₃) δ 16.52 (s, 1H), 7.40-7.33 (m, 4H), 7.30-7.20 (m, 6H), 7.20-7.13 (m, 2H), 7.09-7.02 (m, 4H), 6.56 (d, J=10.4 Hz, 1H), 5.31, 5.26 (ABq, J=16.8 Hz, 2H), 5.17, 5.04 (ABq, J=10.4 Hz, 2H), 4.26, 4.11 (ABq, J=14.0 Hz, 2H), 3.82 (s, 1H), 2.82 (dd, J=15.3, 4.3 Hz, 1H), 2.64-2.52 (m, 1H), 2.52-2.44 (m, 1H), 2.08-1.92 (m, 4H), 0.67 (s, 9H), 0.12 (s, 3H), 0.00 (s, 3H); MS (ESI) m/z 892.56 (M+H).

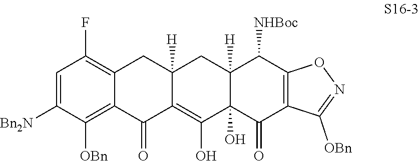

Compound S16-3 was prepared from compound S16-2-2 (884 mg, 0.99 mmol, 1 eq) using General Procedure C, followed by treatment with Boc₂O (227 mg, 1.04 mmol, 1.05 eq) in DCM (10 mL) at 0° C., followed by warming to ambient temperature until complete by LCMS analysis. The reaction solution was diluted with saturated aqueous ammonium chloride (30 mL) and extracted with EtOAc (2×35 mL). The combined organic layers were washed with brine, dried over Na₂SO₄, filtered and concentrated under reduced pressure. The resulting crude product was purified via flash column chromatography on silica gel using 8%-50% EtOAc/hexanes to yield the desired product S16-3 (750 mg, 86%): ¹H NMR (400 MHz, CDCl₃) δ 16.03 (s, 1H), 7.50-7.21 (m, 15H), 7.18-7.11 (m, 5H), 6.68 (d, J=10.4 Hz, 1H), 5.83-5.77 (m, 1H), 5.35 (s, 2H), 5.23 (d, J=9.7 Hz, 1H), 5.13-5.03 (m, 2H), 4.57 (s, 1H), 4.33 (d, J=14.6 Hz, 2H), 4.22 (d, J=14.0 Hz, 2H), 2.92-2.85 (m, 1H), 2.70-2.57 (m, 2H), 2.16-2.05 (m, 2H), 1.57 (s, 9H); MS (ESI) m/z 878.61 (M+H).

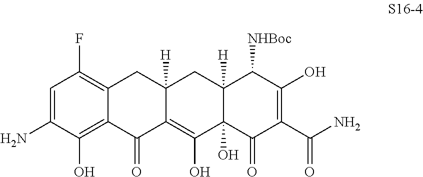 [SMILES: CC(C)(C)OC(=O)N[C@@H]1C(O)=C(C(N)=O)C(=O)[C@@]2(O)C(O)=C3C(=O)c4c(O)c(N)cc(F)c4C[C@H]3C[C@@H]12]

Compound S16-4 was prepared by dissolving S16-3 (750 mg, 0.854 mmol, 1 eq) in methanol:dioxane (1:1, 8 mL) with 1 N aqueous HCl (854 μL, 1 eq). Pd—C (10 wt %, 106 mg) was added in one portion and the reaction vessel was sealed and purged with hydrogen by briefly evacuating the flask followed by flushing with hydrogen gas (1 atm). The reaction mixture was stirred under a hydrogen atmosphere (1 atm) at rt for 6.5 hr. The reaction was filtered through a small Celite pad. The cake was washed with CH₃OH. The filtrate was concentrated and the resulting orange foam was used without further purification. S16-4: MS (ESI) m/z 518.26 (M−H).

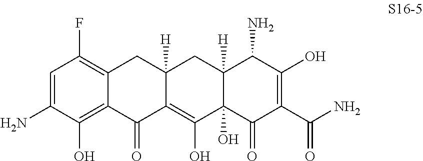 [SMILES: NC(=O)C1=C(O)[C@@H](N)[C@@H]2C[C@@H]3Cc4c(F)cc(N)c(O)c4C(=O)C3=C(O)[C@]2(O)C1=O]

To a solution of S16-4 (20 mg, 0.038 mmol, 1 eq) in CH₃OH (750 μL) was added concentrated HCl (12N, 200 μL). The reaction was stirred at room temperature for 4 hr. The solution was concentrated under reduced pressure and the residue was dissolved in 0.05 NHCl in water and the resulting solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 N HCl/water; Solvent B: CH₃CN; injection volume: 2.0 mL (0.05 N HCl/water); gradient: 5→30% B in A over 20 min; mass-directed fraction collection]. Fractions containing the desired product were collected and freeze-dried to yield compound S16-5: ¹H NMR (400 MHz, CD₃OD, dihydrochloride salt) S 7.38 (d, J=8.6 Hz, 1H), 3.88 (s, 1H), 3.23-3.10 (m, 1H), 3.09-2.95 (m, 1H), 2.64 (d, J=12.2 Hz, 1H), 2.42-2.30 (m, 1H), 2.29-2.19 (m, 1H), 2.68-2.45 (m, 1H); MS (ESI) m/z 420.2 (M+H).

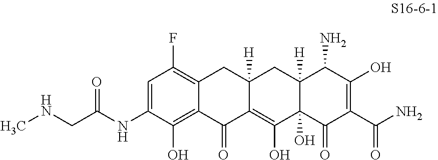 [SMILES: CNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

General Procedure H (acylation/amine addition): To a solution of S16-4 (32 mg, 0.62 mmol, 1 eq) in DMPU:CH₃CN (400 μL:1.6 mL) was added Na₂CO₃ (32 mg, 0.302 mmol, 5 eq) and bromoacetylbromide (6.5 μL, 0.72 mmol, 1.2 eq). This mixture was stirred under an atmosphere of nitrogen for 1.5 hr. A solution of methylamine (2.0 M in THF, 335 μL, 0.62 mmol, 10 eq) was added and the reaction was stirred at room temperature for 17 hr. The reaction solution was concentrated under reduced pressure, then dissolved in CH₃OH (400 μL) and added dropwise to rapidly stirring MTBE (15 mL). The resulting green precipitate was filtered off on a Celite pad and washed with MTBE. The solid was washed off the Celite pad with CH₃OH containing several drops of concentrated HCl. The resulting orange solution was concentrated in vacuo. The crude residue was dissolved in CH₃OH (1 mL), to which was added 0.05 NHCl in water (300 μL) and concentrated HCl (200 μL). The reaction solution was stirred at room temperature for 1.5 hr. The solution was concentrated under reduced pressure and the resulting residue was dissolved in CH₃OH (800 μL) and added to rapidly stirring MTBE (15 mL). The resulting orange precipitate was filtered through a Celite pad and washed as before, then washed off the Celite pad with CH₃OH. The solution was concentrated under reduced pressure. The residue was dissolved in 0.05 N HCl in water and the resulting solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 NHCl/water; Solvent B: CH₃CN; injection volume: 2.0 mL (0.05 N HCl/water); gradient: 5→30% B in A over 20 min; mass-directed fraction collection]. Fractions containing the desired product were collected and freeze-dried to yield compound S16-6-1 (4.9 mg, 4%): ¹H NMR (400 MHz, CD₃OD, dihydrochloride salt) δ 8.22 (d, J=11.0 Hz, 1H), 4.06 (s, 2H), 3.88 (s, 1H), 3.18-3.10 (m, 1H), 3.07-2.93 (m, 1H), 2.77 (s. 3H), 2.62 (d, J=12.8 Hz, 1H), 2.33-2.18 (m, 2H), 1.64-1.56 (m, 1H); MS (ESI) m/z 491.21 (M+H).

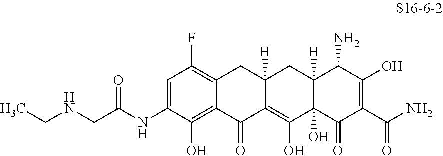 [SMILES: CCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-2 was prepared from compound S16-4 with ethylamine using General Procedure H: ¹H NMR (400 MHz, CD₃OD, dihydrochloride salt) δ 8.22 (d, J=11.0 Hz, 1H), 4.07 (s, 2H), 3.88 (s, 1H), 3.18-3.10 (m, 3H), 3.07-2.93 (m, 1H), 2.67-2.60 (m. 1H), 2.33-2.20 (m, 2H), 1.64-1.56 (m, 1H) 1.35 (t, J=7.3 Hz, 3H); MS (ESI) m/z 565.19 (M+H).

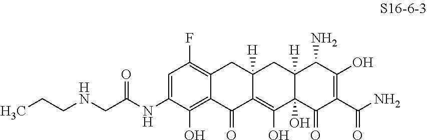 [SMILES: CCCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-3 was prepared from compound S16-4 with propylamine using General Procedure H: ¹H NMR (400 MHz, CD₃OD, dihydrochloride salt) δ 8.22 (d, J=11.0 Hz, 1H), 4.08 (s, 2H), 3.89 (s, 1H), 3.17-2.92 (m, 4H), 2.66 (d, J=12.2 Hz, 1H), 2.33-2.20 (m, 2H), 1.85-1.72 (m, 2H), 1.64-1.56 (m, 1H), 1.04 (t, J=7.6 Hz, 3H); MS (ESI) m/z 519.26 (M+H).

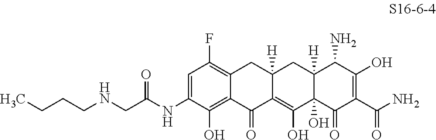 [SMILES: CCCCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-4 was prepared from compound S16-4 with butylamine using General Procedure H: ¹H NMR (400 MHz, CD₃OD, dihydrochloride salt) δ 8.22 (d, J=11.0 Hz, 1H), 4.08 (s, 2H), 3.88 (s, 1H), 3.18-2.94 (m, 4H), 2.66 (d, J=12.2 Hz, 1H), 2.33-2.20 (m, 2H), 1.78-1.68 (m, 2H), 1.64-1.52 (m, 1H), 1.48-1.38 (m, 2H), 1.00 (t, J=7.6 Hz, 3H); MS (ESI) m/z 533.32 (M+H).

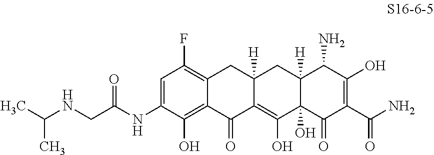 [SMILES: CC(C)NCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-5 was prepared from compound S16-4 with isopropylamine using General Procedure H: ¹H NMR (400 MHz, CD₃OD, dihydrochloride salt) δ 8.23 (d, J=11.0 Hz, 1H), 4.08 (s, 2H), 3.88 (s, 1H), 3.52-3.43 (m, 1H), 3.18-3.10 (m, 1H), 3.05-2.95 (m, 1H), 2.63 (d, J=12.8 Hz, 1H), 2.35-2.20 (m, 2H), 1.64-1.56 (m, 1H), 1.37 (d, J=6.8 Hz, 6H); MS (ESI) m/z 519.19 (M+H).

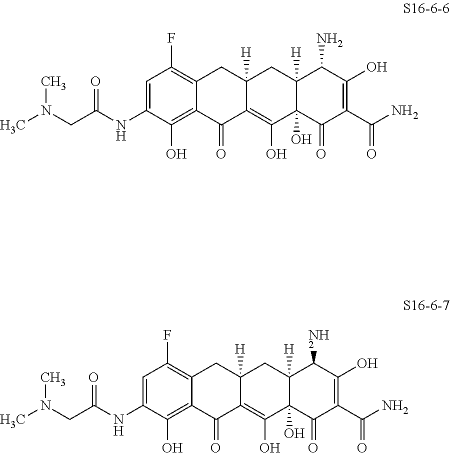 [SMILES: CN(C)CC(=O)Nc1cc(F)c2c(c1O)C(=O)C1=C(O)[C@]3(O)C(=O)C(C(N)=O)=C(O)[C@@H](N)[C@@H]3C[C@@H]1C2 ; CN(C)CC(=O)Nc1cc(F)c2c(c1O)C(=O)C1=C(O)[C@]3(O)C(=O)C(C(N)=O)=C(O)[C@H](N)[C@@H]3C[C@@H]1C2]

Compounds S16-6-6 and S16-6-7 were prepared from compound S16-4 with dimethylamine using General Procedure H. S16-6-6: ¹H NMR (400 MHz, CD₃OD, dihydrochloride salt) δ 8.22 (d, J=11.0 Hz, 1H), 4.22 (s, 2H), 3.87 (s, 1H), 3.18-3.10 (m, 1H), 3.07-2.93 (m, 7H), 2.77 (s. 3H), 2.64-2.60 (m, 1H), 2.33-2.18 (m, 2H), 1.64-1.56 (m, 1H); MS (ESI) 505.27 m/z (M+H). S16-6-7: ¹H NMR (400 MHz, CD₃OD, dihydrochloride salt) δ 8.22 (d, J=11.0 Hz, 1H), 4.78-4.74 (m, 1H), 4.22 (s, 2H), 3.18-3.08 (m, 1H), 2.99 (s, 6H), 2.92-2.74 (m, 2H), 2.36-2.27 (s. 1H), 2.14-2.05 (m, 1H), 1.52-1.42 (m, 1H); MS (ESI) m/z 505.27 (M+H).

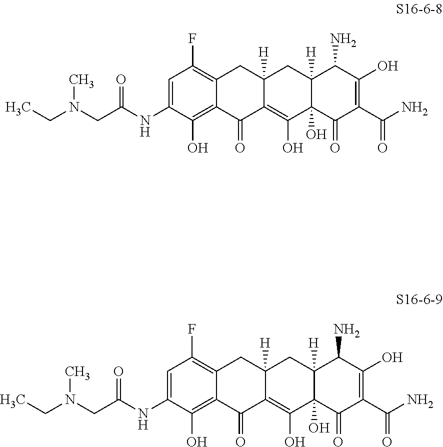 [SMILES: CCN(C)CC(=O)Nc1cc(F)c2c(c1O)C(=O)C1=C(O)[C@]3(O)C(=O)C(C(N)=O)=C(O)[C@@H](N)[C@@H]3C[C@@H]1C2 ; CCN(C)CC(=O)Nc1cc(F)c2c(c1O)C(=O)C1=C(O)[C@]3(O)C(=O)C(C(N)=O)=C(O)[C@H](N)[C@@H]3C[C@@H]1C2]

Compounds S16-6-8 and S16-6-9 were prepared from compound S16-4 with dimethylamine using General Procedure H. S16-6-8: ¹H NMR (400 MHz, CD₃OD, dihydrochloride salt) δ 8.22 (d, J=11.0 Hz, 1H), 4.28 (d, J=17.7 Hz, 1H), 4.16 (d, J=17.7 Hz, 1H), 3.88 (s, 1H), 3.50-3.23 (m, 2H), 3.17-3.10 (m, 1H), 3.03-2.94 (m. 4H), 2.64-2.60 (m, 1H) 2.36-2.19 (m, 2H), 1.66-1.55 (m, 1H), 1.38 (t, J=7.3 Hz, 3H); MS (ESI) m/z 519.26 (M+H). S16-6-9: ¹H NMR (400 MHz, CD₃OD, dihydrochloride salt) δ 8.22 (d, J=11.0 Hz, 1H), 4.78-4.74 (m, 1H), 4.28 (d, J=17.7 Hz, 1H), 4.16 (d, J=17.7 Hz, 1H), 3.50-3.23 (m, 2H), 3.17-3.10 (m, 1H), 3.03-3.73 (m, 6H), 2.37-2.26 (m, 1H), 2.15-2.05 (m, 1H), 1.51-1.35 (m, 4H); MS (ESI) m/z 519.26 (M+H).

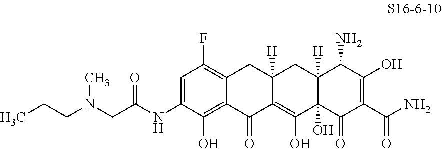 [SMILES: CCCN(C)CC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-10 was prepared from compound S16-4 with N-methylpropylamine using General Procedure H: ¹H NMR (400 MHz, CD₃OD, dihydrochloride salt) δ 8.22 (d, J=11.0 Hz, 1H), 4.29 (d, J=16.5 Hz, 1H), 4.18 (d, J=18.9 Hz, 1H), 3.30-3.12 (m, 2H), 3.15-2.92 (m, 4H), (d, J=12.2 Hz, 1H), 2.36-2.20 (m, 2H), 1.86-1.76 (m, 2H), 1.64-1.56 (m, 1H), 1.03 (t, J=7.3 Hz, 3H); MS (ESI) m/z 533.23 (M+H).

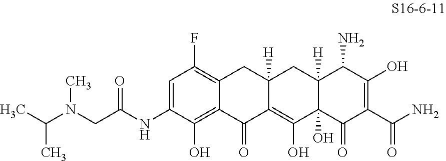 [SMILES: CC(C)N(C)CC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-11 was prepared from compound S16-4 with N-methylisopropylamine using General Procedure H: ¹H NMR (400 MHz, CD₃OD, dihydrochloride salt) δ 8.23 (d, J=11.0 Hz, 1H), 4.30 (d, J=15.9 Hz, 1H), 4.09 (d, J=15.9 Hz, 1H), 3.88 (s, 1H), 3.72-3.65 (m, 1H), 3.18-3.10 (m, 1H), 3.05-2.93 (m, 1H), 2.90 (s, 3H), 2.66-2.61 (m, 1H), 2.35-2.18 (m, 2H), 1.59-1.52 (m, 1H), 1.43-1.32 (m, 6H); MS (ESI) m/z 533.25 (M+H).

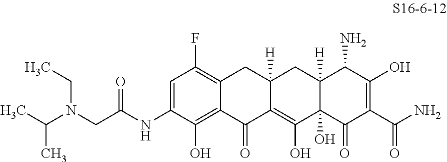 [SMILES: CCN(CC(=O)Nc1cc(F)c2c(c1O)C(=O)C1=C(O)[C@]3(O)C(=O)C(C(N)=O)=C(O)[C@@H](N)[C@@H]3C[C@@H]1C2)C(C)C]

Compound S16-6-12 was prepared from compound S16-4 with N-ethylisopropylamine using General Procedure H: ¹H NMR (400 MHz, CD₃OD, dihydrochloride salt) δ 8.22 (d, J=11.0 Hz, 1H), 4.31 (d, J=17.1 Hz, 1H), 4.08 (d, J=16.5 Hz, 1H), 3.88 (s, 1H), 3.82-3.72 (m, 1H), 3.41-3.32 (m, 1H), 3.21-3.10 (m, 1H), 3.17-2.93 (m, 1H), 2.66-2.61 (m, 1H), 2.35-2.18 (m, 2H), 1.64-1.52 (m, 1H), 1.44-1.30 (m, 9H); MS (ESI) m/z 547.26 (M+H).

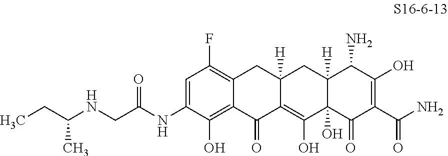 [SMILES: CC[C@@H](C)NCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-13 was prepared from compound S16-4 with R-(−)-sec-butylamine using General Procedure H: ¹H NMR (400 MHz, CD₃OD, dihydrochloride salt) δ 8.24 (d, J=11.0 Hz, 1H), 4.10 (s, 2H), 3.88 (s, 1H), 3.72-3.65 (m, 1H), 3.31-3.25 (m, 2H), 3.18-3.10 (m, 1H), 3.05-2.93 (m, 1H), 2.90 (s, 3H), 2.66-2.61 (m, 1H), 2.35-2.18 (m, 2H), 1.88-1.82 (m, 1H), 1.65-1.52 (m, 1H), 1.38-1.25 (m, 3H), 1.04 (t, J=7.9 Hz, 3H); MS (ESI) m/z 533.23 (M+H).

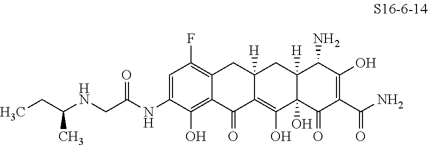 [SMILES: CC[C@H](C)NCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-14 was prepared from compound S16-4 with S-(+)-sec-butylamine using General Procedure H: ¹H NMR (400 MHz, CD₃OD, dihydrochloride salt) δ 8.23 (d, J=11.0 Hz, 1H), 4.09 (s, 2H), 3.87 (s, 1H), 3.18-3.10 (m, 1H), 3.05-2.92 (m, 1H), 2.65-2.60 (m, 1H), 2.36-2.18 (m, 2H), 1.94-1.80 (m, 1H), 1.66-1.53 (m, 2H), 1.33 (d, J=6.7 Hz, 3H), 1.03 (t, J=7.3 Hz, 3H); MS (ESI) m/z 533.23 (M+H).

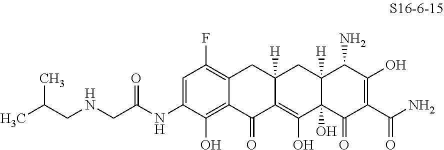 [SMILES: CC(C)CNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-15 was prepared from compound S16-4 with isobutylamine using General Procedure H: ¹H NMR (400 MHz, CD₃OD, dihydrochloride salt) δ 8.24 (d, J=11.0 Hz, 1H), 4.09 (s, 2H), 3.89 (s, 1H), 3.18-3.10 (m, 1), 3.15-2.92 (m, 3H), 2.67-2.60 (m, 1H), 2.34-2.19 (m, 2H), 2.13-2.00 (m, 1H), 1.66-1.52 (m, 1H), 1.06 (d, J=6.7 Hz, 6H); MS (ESI) m/z 533.32 (M+H).

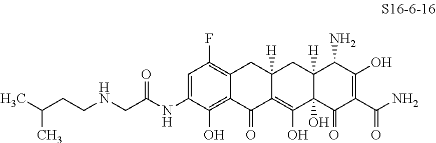 [SMILES: CC(C)CCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-16 was prepared from compound S16-4 with isoamylamine using General Procedure H: ¹H NMR (400 MHz, CD₃OD, dihydrochloride salt) δ 8.23 (d, J=11.0 Hz, 1H), 4.08 (s, 2H), 3.88 (s, 1H), 3.20-3.08 (m, 3H), 3.15-2.92 (m, 1H), 2.68-2.62 (m, 1H), 2.36-2.20 (m, 2H), 1.78-1.52. (m, 3H), 0.99 (d, J=6.1 Hz, 6H); MS (ESI) m/z 547.25 (M+H).

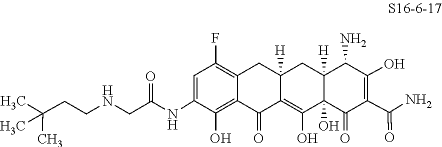 [SMILES: CC(C)(C)CCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-17 was prepared from compound S16-4 with 3,3-dimethylbutylamine using General Procedure H: ¹H NMR (400 MHz, CD₃OD, dihydrochloride salt) δ 8.23 (d, J=11.0 Hz, 1H), 4.10 (s, 2H), 3.89 (s, 1H), 3.19-3.09 (m, 3H), 3.15-2.92 (m, 1H), 2.68-2.62 (m, 1H), 2.35-2.20 (m, 2H), 1.68-1.56 (m, 3H), 0.99 (s, 9H); MS (ESI) m/z 561.27 (M+H).

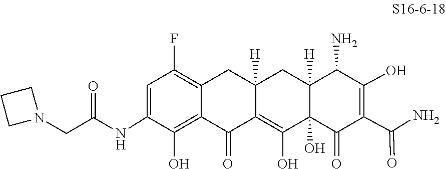 [SMILES: NC(=O)C1=C(O)[C@@H](N)[C@@H]2C[C@@H]3Cc4c(F)cc(NC(=O)CN5CCC5)c(O)c4C(=O)C3=C(O)[C@]2(O)C1=O]

Compound S16-6-18 was prepared from compound S16-4 with azetidine using General Procedure H: ¹H NMR (400 MHz, CD₃OD, dihydrochloride salt) δ 8.18 (d, J=11.0 Hz, 1H), 4.42-4.30 (m, 4H), 4.27-4.10 (m, 2H), 3.87 (s, 1H), 3.19-3.10 (m, 1H), 3.02-2.92 (m, 1H), 2.71-2.59 (m, 2H), 2.53-2.40 (m, 1H), 2.34-2.17 (m, 2H), 1.64-1.52 (m, 1H); MS (ESI) m/z 517.27 (M+H).

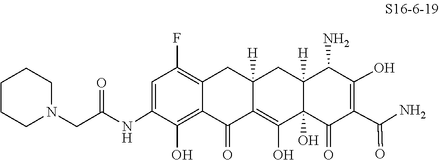 [SMILES: NC(=O)C1=C(O)[C@@H](N)[C@@H]2C[C@@H]3Cc4c(F)cc(NC(=O)CN5CCCCC5)c(O)c4C(=O)C3=C(O)[C@]2(O)C1=O]

Compound S16-6-19 was prepared from compound S16-4 with piperidine using General Procedure H: ¹H NMR (400 MHz, CD₃OD, dihydrochloride salt) δ 8.22 (d, J=11.0 Hz, 1H), 4.19 (s, 2H), 3.88 (s, 1H), 3.77-3.58 (m, 2H), 3.20-3.08 (m, 3H), 3.07-2.94 (m, 1H), 2.68-2.62 (m, 1H), 2.35-2.20 (m, 2H), 2.00-1.82 (m, 5H), 1.65-1.52 (m, 2H); MS (ESI) m/z 545.25 (M+H).

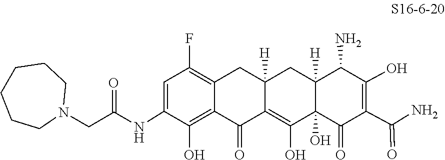 [SMILES: NC(=O)C1=C(O)[C@@H](N)[C@@H]2C[C@@H]3Cc4c(F)cc(NC(=O)CN5CCCCCC5)c(O)c4C(=O)C3=C(O)[C@]2(O)C1=O]

Compound S16-6-20 was prepared from compound S16-4 with hexamethyleneimine using General Procedure H: ¹H NMR (400 MHz, CD₃OD, dihydrochloride salt) δ 8.24 (d, J=11.0 Hz, 1H), 4.27 (s, 2H), 3.89 (s, 1H), 3.61-3.51 (m, 2H), 3.41-3.32 (m, 2H), 3.19-3.09 (m, 1H), 3.07-2.94 (m, 1H), 2.66-2.61 (m, 1H), 2.35-2.20 (m, 2H), 2.06-1.90 (m, 4H), 1.86-1.69 (m, 4H), 1.67-1.53 (m, 1H); MS (ESI) m/z 559.56 (M+H).

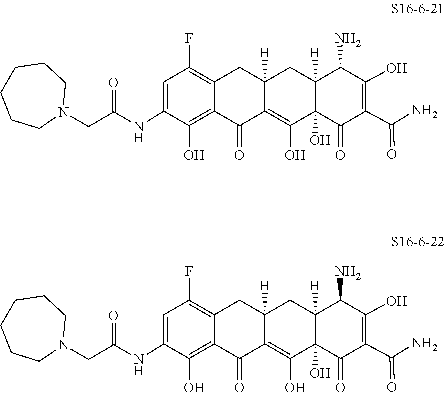 [SMILES: NC(=O)C1=C(O)[C@@H](N)[C@@H]2C[C@@H]3Cc4c(F)cc(NC(=O)CN5CCCCCC5)c(O)c4C(=O)C3=C(O)[C@]2(O)C1=O ; NC(=O)C1=C(O)[C@H](N)[C@@H]2C[C@@H]3Cc4c(F)cc(NC(=O)CN5CCCCCC5)c(O)c4C(=O)C3=C(O)[C@]2(O)C1=O]

Compound S16-6-21 and S16-6-22 were prepared from compound S16-4 with heptamethyleneimine using General Procedure H. S16-6-21: ¹H NMR (400 MHz, CD₃OD, dihydrochloride salt) δ 8.23 (d, J=11.0 Hz, 1H), 4.27 (s, 2H), 3.88 (s, 1H), 3.58-3.45 (m, 2H), 3.43-3.32 (m, 2H), 3.18-3.09 (m, 1H), 3.05-2.92 (m, 1H), 2.68-2.59 (m, 1H), 2.36-2.18 (m, 2H), 2.10-1.90 (m, 4H), 1.88-1.52 (m, 7H); MS (ESI) m/z 573.59 (M+H). S16-6-22: ¹H NMR (400 MHz, CD₃OD, dihydrochloride salt) δ 8.23 (d, J=11.0 Hz, 1H), 4.74 (d, J=4.9 Hz, 1H), 4.25 (s, 2H), 3.56-3.45 (m, 2H), 3.41-3.31 (m, 2H), 3.16-3.07 (m, 1H), 2.92-2.74 (m, 2H), 2.37-2.26 (m, 1H), 2.12-1.89 (m, 5H), 1.86-1.61 (m, 5H), 1.51-1.40 (m, 1H); MS (ESI) m/z 573.59 (M+H).

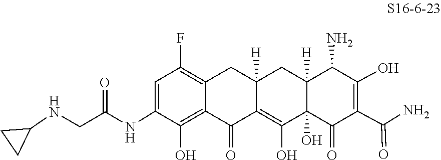 [SMILES: NC(=O)C1=C(O)[C@@H](N)[C@@H]2C[C@@H]3Cc4c(F)cc(NC(=O)CNC5CC5)c(O)c4C(=O)C3=C(O)[C@]2(O)C1=O]

Compound S16-6-23 was prepared from compound S16-4 with cyclopropylamine using General Procedure H. ¹H NMR (400 MHz, CD₃OD, dihydrochloride salt) δ 8.21 (d, J=11.0 Hz, 1H), 4.18 (s, 2H), 3.88 (s, 1H), 3.18-3.08 (m, 1H), 3.05-2.93 (m, 1H), 2.90-2.81 (m, 1H), 2.67-2.62 (m, 1H), 2.33-2.19 (m, 2H), 1.64-1.53 (m, 1H), 0.98-0.89 (m, 4H); MS (ESI) m/z 517.27 (M+H).

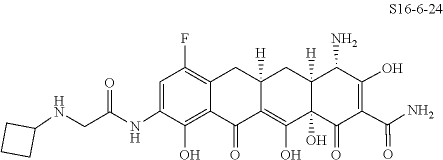 [SMILES: NC(=O)C1=C(O)[C@@H](N)[C@@H]2C[C@@H]3Cc4c(F)cc(NC(=O)CNC5CCC5)c(O)c4C(=O)C3=C(O)[C@]2(O)C1=O]

Compound S16-6-24 was prepared from compound S16-4 with cyclobutylamine using General Procedure H. ¹H NMR (400 MHz, CD₃OD, dihydrochloride salt) δ 8.23 (d, J=11.0 Hz, 1H), 3.96 (s, 2H), 3.91-3.79 (m, 2H), 3.19-3.09 (m, 1H), 3.05-2.92 (m, 1H), 2.68-2.60 (m, 1H), 2.40-2.19 (m, 6H), 2.00-1.88 (m, 2H), 1.65-1.53 (m, 1H); MS (ESI) m/z 531.37 (M+H).

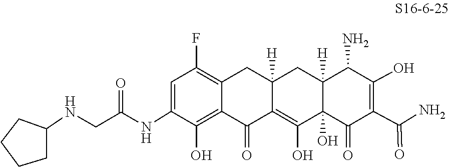 [SMILES: NC(=O)C1=C(O)[C@@H](N)[C@@H]2C[C@@H]3Cc4c(F)cc(NC(=O)CNC5CCCC5)c(O)c4C(=O)C3=C(O)[C@]2(O)C1=O]

Compound S16-6-25 was prepared from compound S16-4 with cyclopentylamine using General Procedure H. ¹H NMR (400 MHz, CD₃OD, dihydrochloride salt) δ 8.25 (d, J=11.0 Hz, 1H), 4.09 (s, 2H), 3.88 (m, 2H), 3.68-3.58 (m, 1H), 3.19-3.09 (m, 1H), 3.05-2.92 (m, 1H), 2.68-2.60 (m, 1H), 2.38-2.12 (m, 4H), 1.91-1.54 (m, 7H); MS (ESI) m/z 545.23 (M+H).

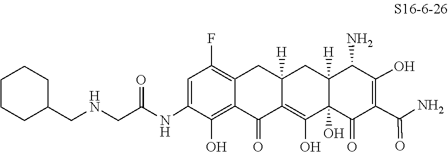 [SMILES: NC(=O)C1=C(O)[C@@H](N)[C@@H]2C[C@@H]3Cc4c(F)cc(NC(=O)CNCC5CCCCC5)c(O)c4C(=O)C3=C(O)[C@]2(O)C1=O]

Compound S16-6-26 was prepared from compound S16-4 with cyclohexanemethylamine using General Procedure H. ¹H NMR (400 MHz, CD₃OD, dihydrochloride salt) δ 8.23 (d, J=11.0 Hz, 1H), 4.07 (s, 2H), 3.87 (m, 2H), 3.19-3.09 (m, 1H), 3.03-2.90 (m, 3H), 2.68-2.60 (m, 1H), 2.38-2.20 (m, 2H), 1.91-1.71 (m, 6H), 1.65-1.55 (m, 1H), 1.42-1.20 (m, 3H), 1.13-1.00 (m, 2H); MS (ESI) m/z 573.26 (M+H).

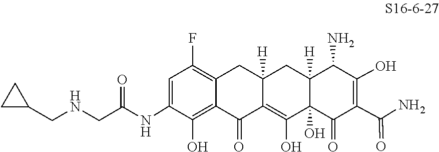 [SMILES: NC(=O)C1=C(O)[C@@H](N)[C@@H]2C[C@@H]3Cc4c(F)cc(NC(=O)CNCC5CC5)c(O)c4C(=O)C3=C(O)[C@]2(O)C1=O]

Compound S16-6-27 was prepared from compound S16-4 with cyclopropanemethylamine using General Procedure H. ¹H NMR (400 MHz, CD₃OD, dihydrochloride salt) δ 8.23 (d, J=11.0 Hz, 1H), 4.10 (s, 2H), 3.87 (m, 2H), 3.19-3.10 (m, 1H), 3.04-2.92 (m, 3H), 2.65-2.60 (m, 1H), 2.34-1.97 (m, 2H), 1.65-1.55 (m, 1H), 1.16-1.08 (m, 1H), 0.78-0.70 (m, 2H), 0.46-0.40 (m, 2H); MS (ESI) m/z 531.21 (M+H).

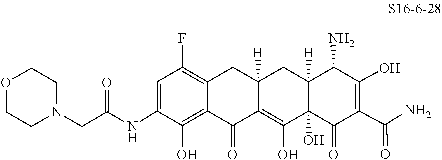 [SMILES: NC(=O)C1=C(O)[C@@H](N)[C@@H]2C[C@@H]3Cc4c(F)cc(NC(=O)CN5CCOCC5)c(O)c4C(=O)C3=C(O)[C@]2(O)C1=O]

Compound S16-6-28 was prepared from compound S16-4 with morpholine using General Procedure H. ¹H NMR (400 MHz, CD₃OD, dihydrochloride salt) δ 8.21 (d, J=11.0 Hz, 1H), 4.26 (s, 2H), 4.13-3.97 (m, 2H), 3.95-3.81 (m, 3H), 3.67-3.51 (m, 2H), 3.38-3.33 (m, 2H), 3.19-3.10 (m, 1H), 3.04-2.92 (m, 3H), 2.65-2.58 (m, 1H), 2.34-1.97 (m, 2H), 1.65-1.55 (m, 1H); MS (ESI) m/z 547.3 (M+H).

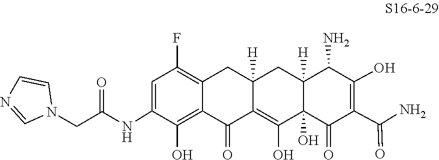 [SMILES: NC(=O)C1=C(O)[C@@H](N)[C@@H]2C[C@@H]3Cc4c(F)cc(NC(=O)Cn5ccnc5)c(O)c4C(=O)C3=C(O)[C@]2(O)C1=O]

Compound S16-6-29 was prepared from compound S16-4 with imidazole using General Procedure H. ¹H NMR (400 MHz, CD₃OD, dihydrochloride salt) δ 8.99 (s, 1H), 8.16 (d, J=10.8 Hz, 1H), 7.67 (s, 1H), 7.60 (s, 1H), 5.32 (s, 2H), 3.87 (s, 1H), 3.17-3.10 (m, 1H), 3.05-2.92 (m, 1H), 2.65-2.58 (m, 1H), 2.34-2.15 (m, 2H), 1.65-1.52 (m, 1H); MS (ESI) m/z 528.15 (M+H).

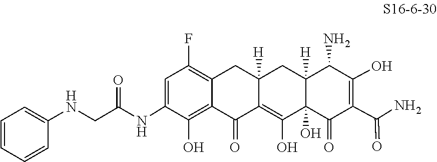 [SMILES: NC(=O)C1=C(O)[C@@H](N)[C@@H]2C[C@@H]3Cc4c(F)cc(NC(=O)CNc5ccccc5)c(O)c4C(=O)C3=C(O)[C@]2(O)C1=O]

Compound S16-6-30 was prepared from compound S16-4 with aniline using General Procedure H. ¹H NMR (400 MHz, CD₃OD, dihydrochloride salt) δ 8.29 (d, J=11.0 Hz, 1H), 7.40-7.32 (m, 2H), 7.11-7.00 (m, 3H), 4.14 (s, 2H), 3.86 (s, 1H), 3.19-3.09 (m, 1H), 3.02-2.90 (m, 1H), 2.65-2.55 (m, 1H), 2.34-2.16 (m, 2H), 1.62-1.52 (m, 1H); MS (ESI) m/z 551.21 (M−H).

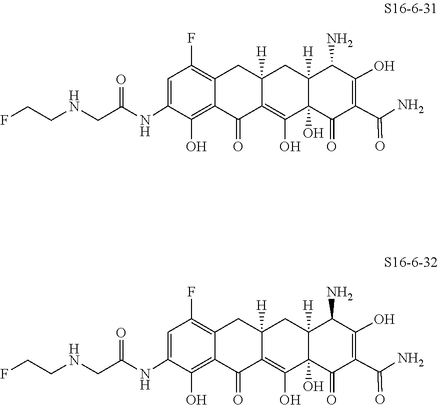 [SMILES: NC(=O)C1=C(O)[C@@H](N)[C@@H]2C[C@@H]3Cc4c(F)cc(NC(=O)CNCCF)c(O)c4C(=O)C3=C(O)[C@]2(O)C1=O ; NC(=O)C1=C(O)[C@H](N)[C@@H]2C[C@@H]3Cc4c(F)cc(NC(=O)CNCCF)c(O)c4C(=O)C3=C(O)[C@]2(O)C1=O]

Compound S16-6-31 and S16-6-32 were prepared from compound S16-4 with 2-fluoroethylamine hydrochloride (4 eq) using General Procedure H. S16-6-31: ¹H NMR (400 MHz, CD₃OD, dihydrochloride salt) δ 8.23 (d, J=11.0 Hz, 1H), 4.88-4.83 (m, 1H), 4.76-4.70 (m, 1H), 4.16 (s, 2H), 3.87 (s, 1H), 3.56-3.44 (m, 2H), 3.19-3.09 (m, 1H), 3.06-2.94 (m, 1H), 2.67-2.57 (m, 1H), 2.34-2.16 (m, 2H), 1.62-1.52 (m, 1H); MS (ESI) δ 23.27 m/z (M+H). S16-6-32: ¹H NMR (400 MHz, CD₃OD, dihydrochloride salt) δ 8.25 (d, J=11.0 Hz, 1H), 4.89-4.81 (m, 1H), 4.78-4.72 (m, 2H), 4.17 (s, 2H), 3.56-3.44 (m, 2H), 3.19-3.09 (m, 1H), 2.98-2.78 (m, 1H), 2.39-2.24-2.67 (m, 1H), 2.14-2.08 (m, 1H), 1.55-1.42 (m, 1H); MS (ESI) m/z 523.27 (M+H).

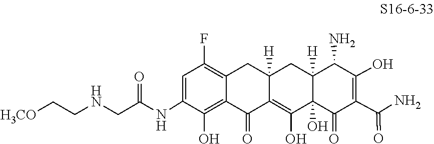 [SMILES: COCCNCC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-33 was prepared from compound S16-4 with 2-methoxyethylamine using General Procedure H. ¹H NMR (400 MHz, CD₃OD, dihydrochloride salt) δ 8.23 (d, J=11.0 Hz, 1H), 4.10 (s, 2H), 3.87 (s, 1H), 3.72-3.67 (m, 2H), 3.42 (s, 3H), 3.35-3.31 (m, 2H), 3.19-3.09 (m, 1H), 3.04-2.92 (m, 1H), 2.65-2.60 (m, 1H), 2.34-2.18 (m, 2H), 1.64-1.52 (m, 1H); MS (ESI) m/z 535.24 (M+H).

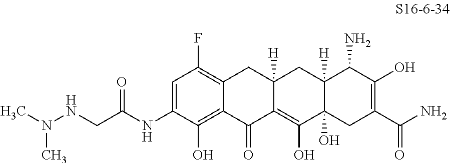 [SMILES: CN(C)NCC(=O)Nc1cc(F)c2c(c1O)C(=O)C1=C(O)[C@]3(O)CC(C(N)=O)=C(O)[C@@H](N)[C@@H]3C[C@@H]1C2]

Compound S16-6-34 was prepared from compound S16-4 with N,N-dimethylethylenediamine using General Procedure H. ¹H NMR (400 MHz, CD₃OD, trihydrochloride salt) δ 8.23 (d, J=11.0 Hz, 1H), 4.21 (s, 2H), 3.87 (s, 1H), 3.67-3.55 (m, 4H), 3.19-3.09 (m, 1H), 3.05-2.92 (m, 7H), 2.65-2.60 (m, 1H), 2.35-2.18 (m, 2H), 1.64-1.52 (m, 1H); MS (ESI) m/z 548.24 (M+H).

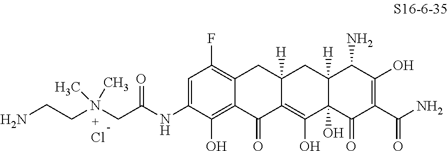

Side-product S16-6-35 was also obtained from the reaction to produce S16-6-34. ¹H NMR (400 MHz, CD₃OD, dihydrochloride salt) δ 8.18 (d, J=10.4 Hz, 1H), 4.54 (s, 2H), 4.10-4.02 (m, 2H), 3.87 (s, 1H), 3.60-6.52 (m, 2H), 3.46 (s, 6H), 3.19-3.10 (m, 1H), 3.04-2.93 (m, 1H), 2.66-2.59 (m, 1H), 2.35-2.17 (m, 2H), 1.65-1.43 (m, 1H); MS (ESI) m/z 548.5 (M+H).

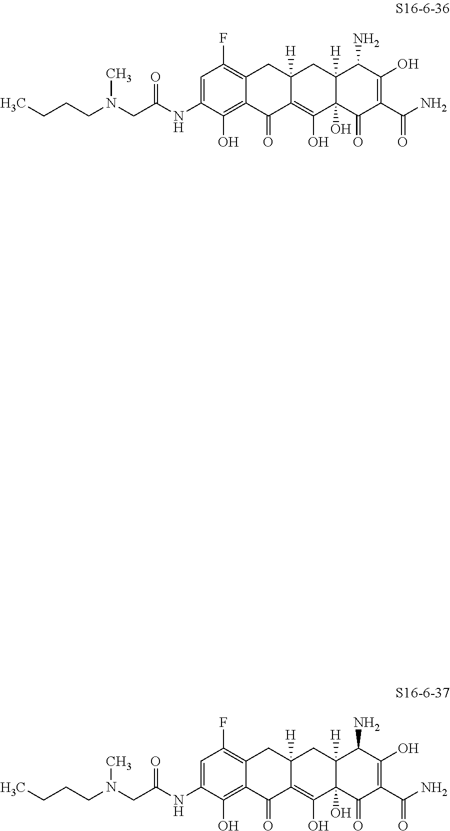 [SMILES: CCCCN(C)CC(=O)Nc1cc(F)c2c(c1O)C(=O)C1=C(O)[C@]3(O)C(=O)C(C(N)=O)=C(O)[C@@H](N)[C@@H]3C[C@@H]1C2 ; CCCCN(C)CC(=O)Nc1cc(F)c2c(c1O)C(=O)C1=C(O)[C@]3(O)C(=O)C(C(N)=O)=C(O)[C@H](N)[C@@H]3C[C@@H]1C2]

Compounds S16-6-36 and S16-6-37 were prepared from compound S16-4 with N-methylbutylamine using General Procedure H. S16-6-36: ¹H NMR (400 MHz, CD₃OD, dihydrochloride salt) δ 8.23 (d, J=11.0 Hz, 1H), 4.31, 4.19 (ABq, J=16.5 Hz, 2H), 3.88 (s, 1H), 3.34-3.25 (m, 1H), 3.23-3.11 (m, 2H), 3.05-2.94 (m, 4H), 2.67-2.60 (m, 1H), 2.36-2.18 (m, 2H), 1.82-1.71 (m, 2H), 1.66-1.54 (m, 1H), 1.50-1.39 (m, 2H), 1.02 (t, J=7.3 Hz, 3H); MS (ESI) m/z 547.26 (M+H). S16-6-37: ¹H NMR (400 MHz, CD₃OD, dihydrochloride salt) δ 8.24 (d, J=11.0 Hz, 1H), 4.76 (d, J=4.9 Hz, 1H), 4.29, 4.19 (ABq, J=16.8 Hz, 2H), 3.41-3.24 (m, 2H), 3.21-3.10 (m, 1H), 2.99 (s, 3H), 2.94-2.70 (m, 2H), 2.38-2.28 (m, 1H), 2.13-2.05 (m, 1H), 1.82-1.71 (m, 2H), 1.52-1.39 (m, 3H), 1.02 (t, J=7.3 Hz, 3H); MS (ESI) m/z 547.26 (M+H).

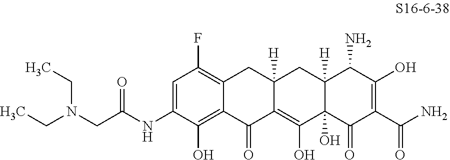 [SMILES: CCN(CC)CC(=O)Nc1cc(F)c2c(c1O)C(=O)C1=C(O)[C@]3(O)C(=O)C(C(N)=O)=C(O)[C@@H](N)[C@@H]3C[C@@H]1C2]

Compound S16-6-38 was prepared from compound S16-4 with diethylamine using General Procedure H. ¹H NMR (400 MHz, CD₃OD, dihydrochloride salt) δ 8.21 (d, J=11.0 Hz, 1H), 4.24 (s, 2H), 3.88 (s, 1H), 3.39-3.30 (m, 4H), 3.14 (dd, J=15.3, 4.3 Hz, 1H), 3.05-2.93 (m, 1H), 2.64 (d, J=12.8 Hz, 1H), 2.35-2.18 (m, 2H), 1.64-1.51 (m, 1H), 1.36 (t, J=7.3 Hz, 6H); MS (ESI) m/z 533.36 (M+H).

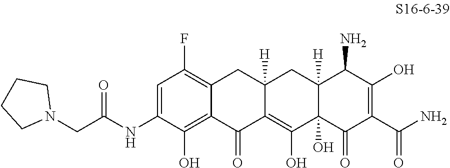 [SMILES: NC(=O)C1=C(O)[C@H](N)[C@@H]2C[C@@H]3Cc4c(F)cc(NC(=O)CN5CCCC5)c(O)c4C(=O)C3=C(O)[C@]2(O)C1=O]

Compound S16-6-39 was prepared from 7-fluoro-9-pyrrolidinoacetamido-6-demethyl-6-deoxytetracycline (J. Med. Chem., 2012, 597-605) in a manner similar to S1-6-2. ¹H NMR (400 MHz, CD₃OD, dihydrochloride salt) δ 8.22 (d, J=11.0 Hz, 1H), 4.74 (d, J=4.9 Hz, 1H), 4.31 (s, 2H), 3.82-3.72 (m, 2H), 3.23-3.06 (m, 3H), 2.94-2.74 (m, 2H), 2.37-2.26 (m, 1H), 2.23-1.99 (m, 5H), 1.52-1.39 (m, 1H); MS (ESI) m/z 531.35 (M+H).

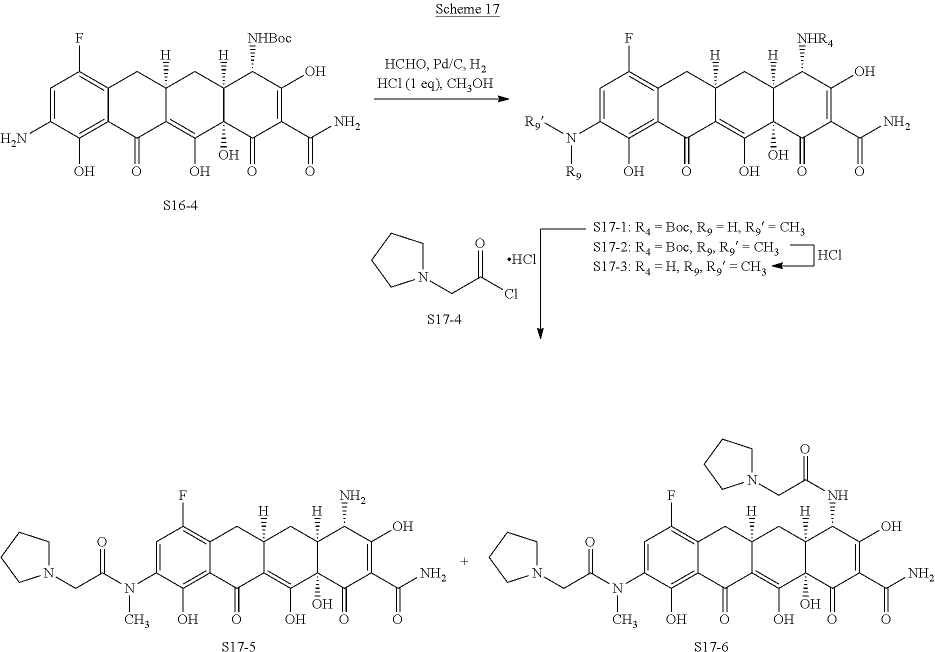

The following compounds were prepared per Scheme 17.

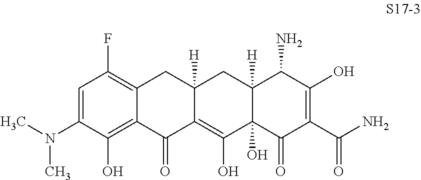 [SMILES: CN(C)c1cc(F)c2c(c1O)C(=O)C1=C(O)[C@]3(O)C(=O)C(C(N)=O)=C(O)[C@@H](N)[C@@H]3C[C@@H]1C2]

To a solution of S16-4 (26.7 mg, 0.051 mmol, 1 eq) in CH₃OH (1 mL) was added 1N aqueous HCl (51 μL, 0.051 mmol, 1 eq), HCHO (aqueous, 37 wt %, 5.7 μL, 0.77 mmol, 1.5 eq) and Pd—C (1 wt %, 15 mg). The reaction vessel was sealed and purged with hydrogen by briefly evacuating the flask followed by flushing with hydrogen gas (1 atm). The reaction mixture was stirred under a hydrogen atmosphere (1 atm) at rt for 2 h 30 min. The reaction was filtered through a small Celite pad. The cake was washed with CH₃OH. The filtrate was concentrated and the crude residue was dissolved in CH₃OH (1 mL), to which was added 0.05 N HCl in water (300 μL) and concentrated HCl (200 μL). The reaction solution was stirred at room temperature for 1.5 hr. The solution was concentrated under reduced pressure and the resulting residue was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 NHCl/water; Solvent B: CH₃CN; injection volume: 3.0 mL (0.05 N HCl/water); gradient: 5→30% B in A over 15 min; mass-directed fraction collection].

Fractions containing the desired product were collected and freeze-dried to yield compound S17-3 (10.8 mg, 40%): ¹H NMR (400 MHz, CD₃OD, dihydrochloride salt) δ 7.91 (d, J=9.8 Hz, 1H), 3.91 (s, 1H), 3.31-3.30 (m, 6H). 3.26-3.18 (m, 1H), 3.12-3.01 (m, 1H), 2.69 (d, J=12.2 Hz, 1H), 2.45-2.34 (m, 1H), 2.32-2.23 (m, 1H), 1.69-1.55 (m, 1H); MS (ESI) m/z 448.25 (M+H).

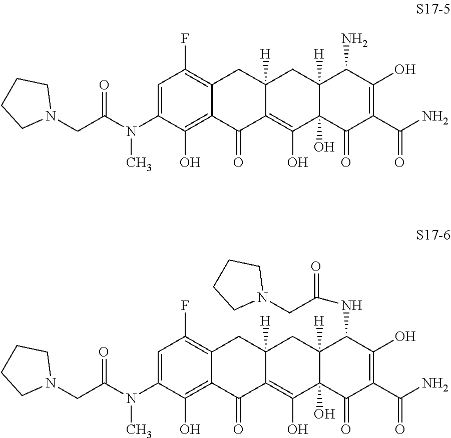 [SMILES: CN(C(=O)CN1CCCC1)c1cc(F)c2c(c1O)C(=O)C1=C(O)[C@]3(O)C(=O)C(C(N)=O)=C(O)[C@@H](N)[C@@H]3C[C@@H]1C2 ; CN(C(=O)CN1CCCC1)c1cc(F)c2c(c1O)C(=O)C1=C(O)[C@]3(O)C(=O)C(C(N)=O)=C(O)[C@@H](NC(=O)CN4CCCC4)[C@@H]3C[C@@H]1C2]

To a solution of S16-4 (17.6 mg, 0.034 mmol, 1 eq) in CH₃OH (1 mL) was added 1N aqueous HCl (34 μL, 0.034 mmol, 1 eq), HCHO (aqueous, 37 wt %, 25 μL of a 10% volume solution in CH₃OH, 0.034 mmol, 1 eq), and Pd—C (10 wt %, 10 mg). The reaction vessel was sealed and purged with hydrogen by briefly evacuating the flask followed by flushing with hydrogen gas (1 atm). The reaction mixture was stirred under a hydrogen atmosphere (1 atm) at rt for 1 h 30 min. The reaction was filtered through a small Celite pad. The cake was washed with CH₃OH. The filtrate was concentrated. The crude residue was dissolved in NMP under nitrogen atmosphere and charged with S17-4 (prepared per literature procedure Org. Process Res. Dev., 2013, 17, 838-845; 10 eq). The reaction solution was added dropwise to rapidly stirring MTBE (15 mL). The resulting tan precipitate was filtered off on a Celite pad and washed with MTBE. The solid was washed off the Celite pad with CH₃OH. The resulting orange solution was concentrated in vacuo. The crude residue was dissolved in CH₃OH (1 mL), to which was added 0.05 NHCl in water (300 μL) and concentrated HCl (200 μL). The reaction solution was stirred at room temperature for 15 hr. The solution was concentrated under reduced pressure and the resulting residue was dissolved in CH₃OH (800 μL) and added to rapidly stirring MTBE (15 mL). The resulting orange precipitate was filtered through a Celite pad and washed as before, then washed off the Celite pad with CH₃OH. The solution was concentrated under reduced pressure. The residue was dissolved in 0.05 N HCl in water and the resulting solution was purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 N HCl/water; Solvent B: CH₃CN; injection volume: 2.0 mL (0.05 N HCl/water); gradient: 5→30% B in A over 20 min; mass-directed fraction collection]. Fractions containing the desired product and those containing a corresponding diacylated compound were collected and freeze-dried to yield compounds S17-5 (5 mg, 24%) and S17-6 (3 mg, 12%). S17-5: ¹H NMR (400 MHz, CD₃OD, dihydrochloride salt) δ 57.53-7.48 (m, 1H), 4.10, 4.05 (ABq, 10.5 Hz, 1H), 3.93-3.83 (m, 2H), 3.79-3.62 (m, 2H), 3.27-3.13 (m, 4H), 3.10-2.93 (m, 3H), 2.70-2.61 (m, 1H), 2.43-1.91 (m, 6H), 1.68-1.52 (m, 1H); MS (ESI) m/z 545.33 (M+H). S17-6: ¹H NMR (400 MHz, CD₃OD, dihydrochloride salt) δ 8.72 (at, J=7.3 Hz, 1H), 7.49 (dd, J=8.5, 2.4 Hz, 1H), 4.78-4.68 (m, 1H), 4.21-4.01 (m, 2H), 3.89, 3.84 (ABq, 8.0 Hz, 1H), 3.81-3.61 (m, 4H), 3.23 (d, J=7.6 Hz, 3H), 3.21-3.10 (m, 3H), 3.10-2.92 (m, 3H), 2.61-2.31 (m, 2H), 2.22-1.92 (m, 9H), 1.73-1.52 (m, 1H); MS (ESI) m/z 656.30 (M+H).

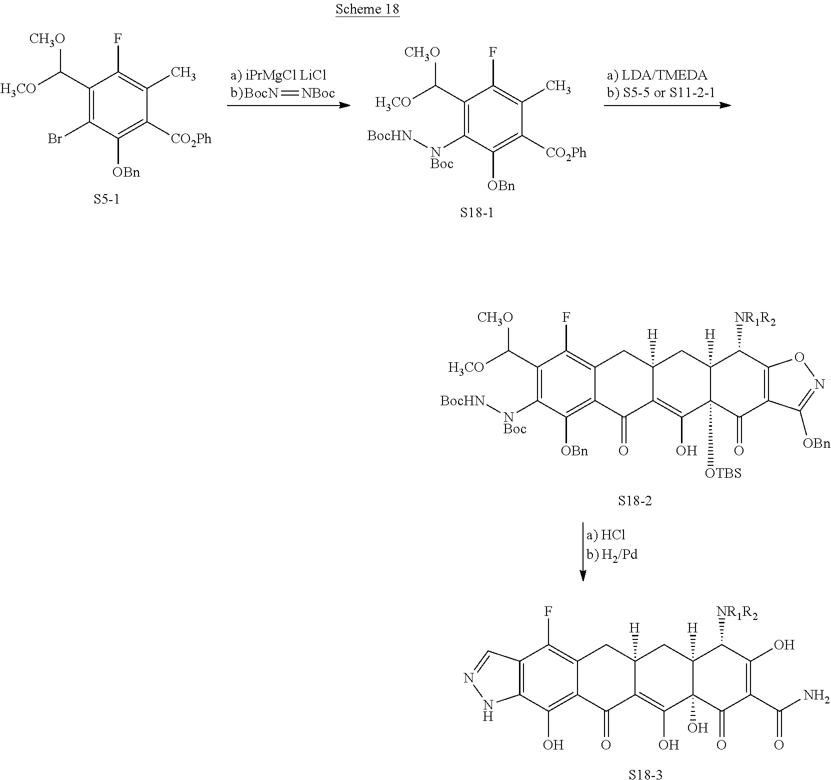

The following compounds were prepared per Scheme 18.

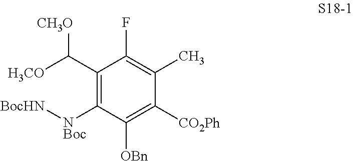

A flame-dried flask was charged with S5-1 (748 mg, 1.53 mmol, 1 eq) under N₂, dissolved in THF (24 mL) and cooled to −78° C. Isopropyl magnesium chloride lithium chloride complex (1.3N in THF, 5.88 mL, 7.64 mmol, 5 eq) was added dropwise to the reaction solution over 15 min, maintaining the internal temperature below −70° C. The anion mixture was allowed to warm slowly to 0° C. over one hour, and was then re-cooled to −78° C. A flame-dried flask was charged with di-tert-butyl azodicarboxylate (1.76 g, 7.63 mmol, 5 eq), evacuated and back-filled with N₂, then dissolved in THF (5 mL). This solution was added dropwise over 30 min to the cold anion solution with a THF rinse forward (1 mL), maintaining the internal temperature below −70° C. The resulting reaction mixture was allowed to warm slowly to room temperature overnight. Saturated aqueous ammonium chloride (12 mL), then water (10 mL) were added and the mixture extracted three times with EtOAc (50 mL, 2×20 mL). The combined organic layers were dried over Na₂SO₄, were filtered, and were concentrated under reduced pressure. The resulting residue was purified via flash column chromatography on silica gel with 2%-25% EtOAc in hexanes as eluent to provide the desired compound S18-1 (746 mg, 76%): ¹H NMR (400 MHz, CDCl₃, rotamers) δ 7.44-7.23 (m, 8H), 7.09-6.76 (m, 2H), 5.99 (m, 0.5H), 5.88 (m, 0.5H), 5.10-5.94 (m, 2H), 3.60-3.43 (m, 6H), 2.40-2.33 (m, 3H), 1.57-1.38 (m, 18H); ); MS (ESI) m/z 641.26 (M+H).

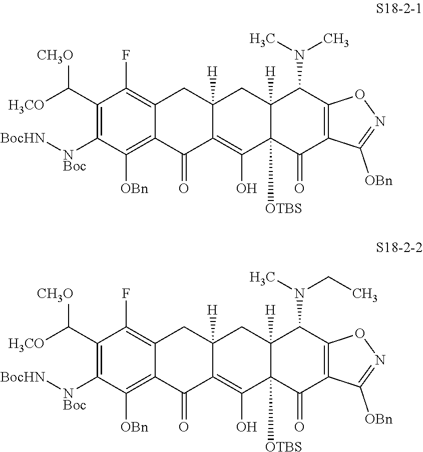

Compounds S18-2-1 and S18-2-2 were prepared from compound S18-1 and dimethylenone S5-5 and ethylmethylenone S11-2-1, respectively, by using General Procedure E. S18-2-1: MS (ESI) m/z 1029.22 (M+H). S18-2-2: MS (ESI) m/z 1043.41 (M+H).

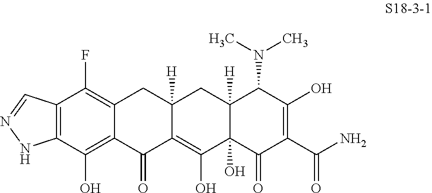 [SMILES: CN(C)[C@@H]1C(O)=C(C(N)=O)C(=O)[C@@]2(O)C(O)=C3C(=O)c4c(c(F)c5cn[nH]c5c4O)C[C@H]3C[C@@H]12]

A solution of S18-2-1 (33 mg, 0.032 mmol, 1 eq) in THF (500 μL) and 4N aqueous HCl (500 μL) was stirred at room temperature overnight, then heated at 50° C. for 3.5 hr. The solution was neutralized via the addition of pH 7 phosphate buffer and the solution was extracted with EtOAc. The combined organic layers were dried over Na₂SO₄, filtered and concentrated under reduced pressure. The crude residue was deprotected using General Procedure D-2 to provide desired compound S18-3-1: ¹H NMR (400 MHz, CD₃OD, dihydrochloride salt) δ 8.05 (s, 1H), 4.08 (s, 1H), 3.39-3.22 (m, 1H), 3.09-2.91 (m, 8H), 2.34-2.17 (m, 2H), 1.70-1.57 (m, 1H); MS (ESI) m/z 472.98 (M+H).

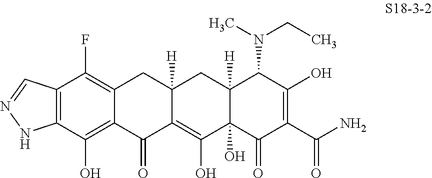 [SMILES: CCN(C)[C@@H]1C(O)=C(C(N)=O)C(=O)[C@@]2(O)C(O)=C3C(=O)c4c(c(F)c5cn[nH]c5c4O)C[C@H]3C[C@@H]12]

A solution of S18-2-2 (207 mg, 0.198 mmol, 1 eq) in THF (3 mL) and 4N aqueous HCl (3 mL) was stirred and heated at 50° C. for 3 hr. The solution was neutralized via careful addition of 6N aqueous NaOH and the solution was extracted with EtOAc. The combined organic layers were dried over Na₂SO₄, filtered and concentrated under reduced pressure. The crude residue was deprotected using General Procedure D-2 to provide desired compound S18-3-2: ¹H NMR (400 MHz, CD₃OD, rotamers, dihydrochloride salt) δ 8.06 (s, 1H), 4.22 (s, 0.5H), 4.12 (s, 0.5H), 3.54-3.42 (m, 1H), 3.40-3.19 (m, 2H), 3.08-2.87 (m, 5H), 2.34-2.17 (m, 2H), 1.72-1.57 (m, 1H), 1.54-1.34 (m, 3H). MS (ESI) m/z 487.09 (M+H).

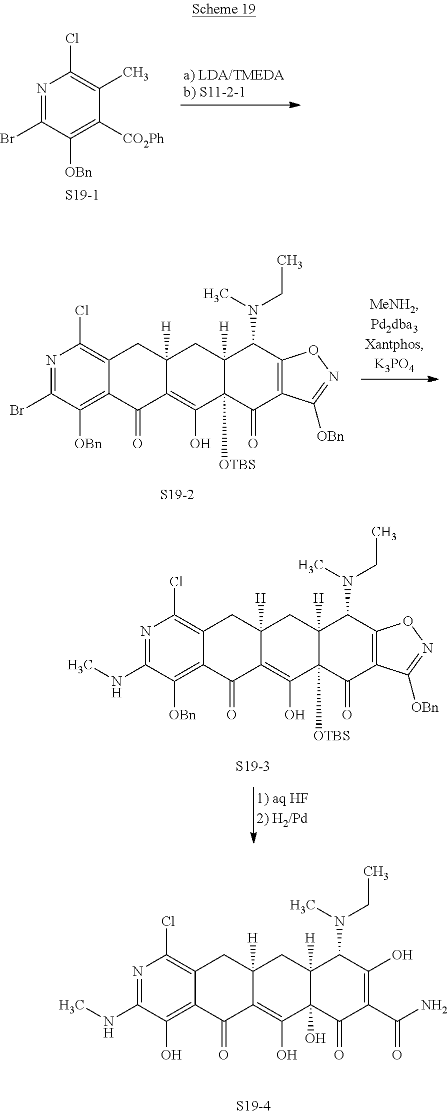

The following compounds were prepared per Scheme 19.

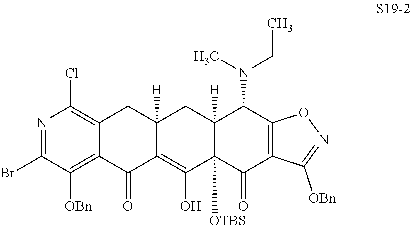 [SMILES: CCN(C)[C@@H]1c2onc(OCc3ccccc3)c2C(=O)[C@@]2(O[Si](C)(C)C(C)(C)C)C(O)=C3C(=O)c4c(c(Cl)nc(Br)c4OCc4ccccc4)C[C@H]3C[C@@H]12]

Compound S19-1 (prepared per literature procedures including WO2010/129055 A1; 518 mg, 1.20 mmol, 1 eq) and ethylmethylenone S11-2-1 (600 mg, 1.21 mmol, 1 eq) were placed under N₂, dissolved in THF (12 mL), and cooled to −73° C. LHMDS (1.0 M in THF, 3.6 mL, 3.6 mmol, 3 eq) was added dropwise over 26 min, maintaining internal temperature below −70° C. The reaction solution was allowed to warm to 0° C. over 1 hr. The solution was neutralized via the addition of pH 7 phosphate buffer (20 mL) and the solution was allowed to warm to room temperature. The solution was extracted with DCM (3×40 mL) and the combined organic layers were washed with 1N NaOH (2×25 mL) and brine, then dried over Na₂SO₄, filtered, and concentrated under reduced pressure. The resulting residue was purified via flash column chromatography on silica gel with 2%-25% EtOAc in hexanes as eluent to provide the desired compound S19-2 (812 mg, 81%): ¹H NMR (400 MHz, CDCl₃, rotamers) δ 15.45 (brs, 1H), 7.54-7.45 (m, 4H), 7.43-7.30 (m, 6H), 5.40-5.30 (m, 2H), 5.03 (aq, J=9.4 Hz, 2H), 3.97-3.86 (m, 1H), 3.24 (dd, J=16.2, 5.2 Hz, 1H), 3.12-3.02 (m, 1H), 2.90-2.75 (m, 1H), 2.72-2.56 (m, 2H), 2.55-2.32 (m, 5H), 2.23-2.11 (m, 1H), 1.19-1.06 (m, 3H), 0.81 (s, 9H), 0.288-0.20 (brm, 3H), 0.13 (s, 3H); MS (ESI) m/z 836.16 (M+H).

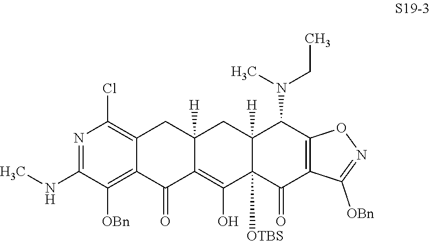 [SMILES: CCN(C)[C@@H]1c2onc(OCc3ccccc3)c2C(=O)[C@@]2(O[Si](C)(C)C(C)(C)C)C(O)=C3C(=O)c4c(c(Cl)nc(NC)c4OCc4ccccc4)C[C@H]3C[C@@H]12]

A sealable vessel was charged with S19-2 (290 mg, 0.317 mmol, 1 eq), Pd₂dba₃ (13.5 mg, 0.015 mmol, 0.05 eq), Xantphos (30.3 mg, 0.052 mmol, 0.15 eq), K₃PO₄ (202 mg, 0.952 mmol, 3 eq). The vessel was capped and sealed, then evacuated and back-filled with N₂ (g) three times. The vessel was charged with 1,4-dioxane (3.2 mL) and methylamine solution (2.0 M in THF, 475 μL, 0.951 mmol, 3 eq) and then placed in a 100° C. bath for 2 hr. The resulting mixture was filtered through a Celite pad with an EtOAc wash. The filtrate was concentrated under reduced pressure. Purification of the resulting residue by preparative reverse phase HPLC on a Waters Autopurification system using a Sunfire Prep C₁₈ OBD column [5 μm, 19×50 mm; flow rate, 20 mL/min; Solvent A: H₂O with 0.1% HCO₂H; Solvent B: CH₃CN with 0.1% HCO₂H; gradient: 5→100% B in A over 20 min; mass-directed fraction collection]. Fractions with the desired MW also contained starting material. Lyophilization of these fractions provided a mixture of S19-2 and S19-3 in a ratio of 1:0.43 (ratio determined via ¹H NMR in CDCl₃; 99 mg total, 28.5 mg desired product, 11%). This mixture was used without further purification. S19-3: MS (ESI) m/z 785.18 (M+H).

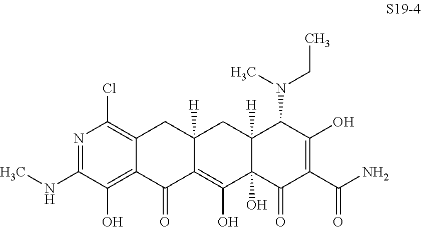 [SMILES: CCN(C)[C@@H]1C(O)=C(C(N)=O)C(=O)[C@@]2(O)C(O)=C3C(=O)c4c(O)c(NC)nc(Cl)c4C[C@H]3C[C@@H]12]

Compound S19-4 was prepared from S19-3 using General Procedures C, and D-2 (in CH₃OH:dioxane 1:1 with no HCl/water). ¹H NMR (400 MHz, CD₃OD, rotamers, dihydrochloride salt) δ 54.22 (s, 0.5H), 4.12 (s, 0.5H), 3.53-3.41 (m, 1H), 3.37-3.29 (m, 1H), 3.10-2.87 (m, 9H), 2.31-2.15 (m, 2H), 1.69-1.53 (m, 1H), 1.45-1.33 (m, 3H). MS (ESI) m/z 493.05 (M+H).

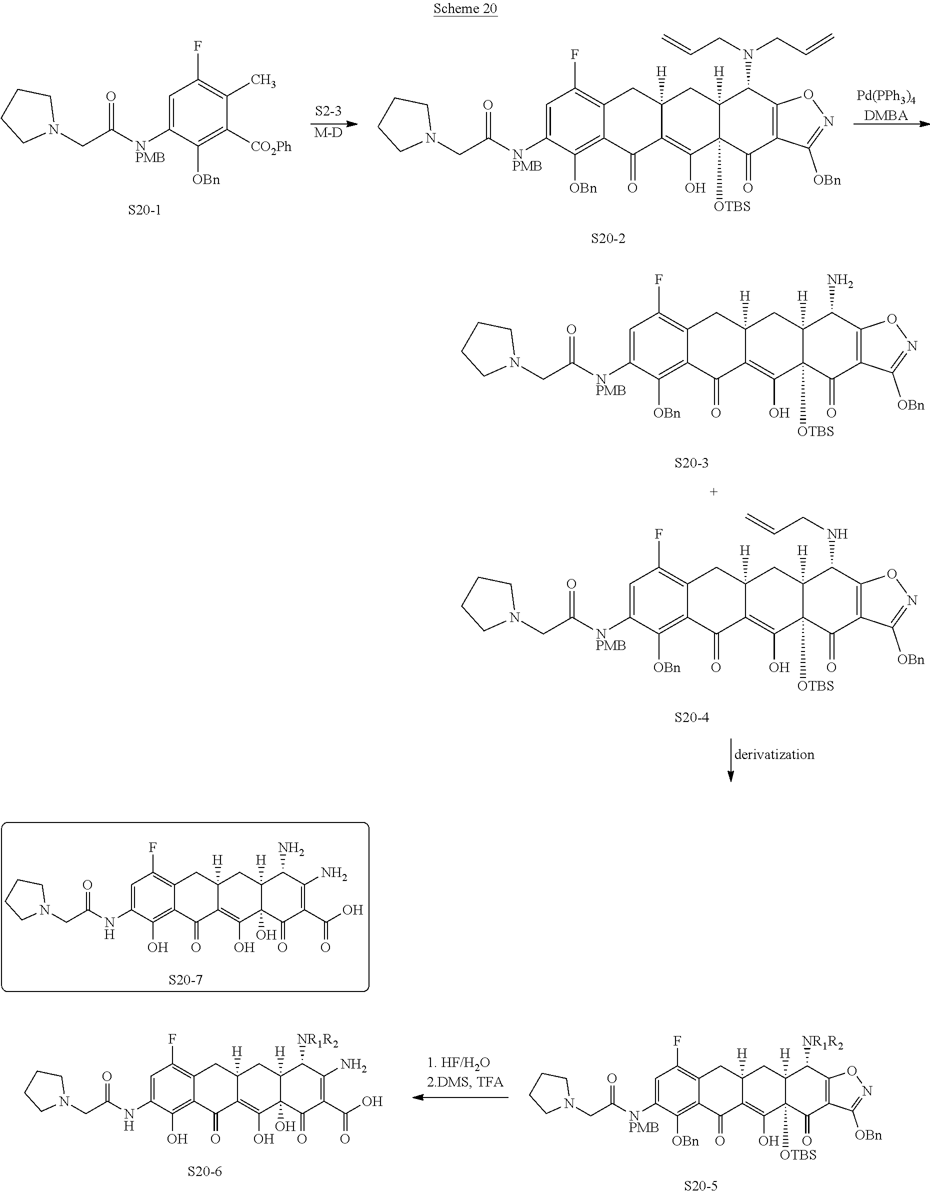

The following compounds were prepared per Scheme 20.

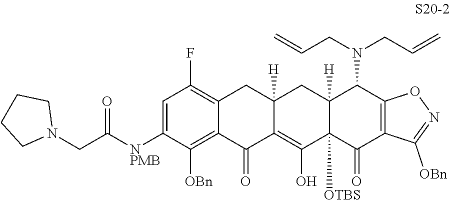

Compound S20-2 was prepared from known D-ring precursor S20-1 (prepared per literature procedure: J. Org. Chem., 2017, 82, 936-943) and S2-3 (observed as a mixture of rotamers via ¹HNMR spectral analysis in CDCl₃) using General Procedure E. S20-2: MS (ESI) m/z 1023.74 (M+H).

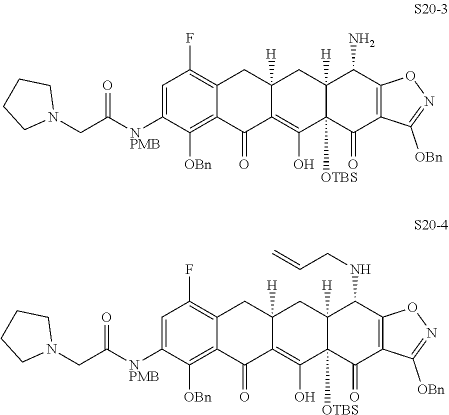

Compounds S20-3 and S20-4 were prepared from compound S20-2 by using General Procedure A. S20-3: MS (ESI) m/z 943.67 (M+H). S20-4: MS (ESI) m/z 983.67 (M+H).

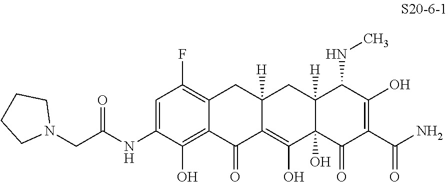 [SMILES: CN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-1 was prepared from compound S20-3 by using General Procedures B-1 with HCHO, C, and D-1. ¹H NMR (400 MHz, CD₃OD, dihydrochloride salt) δ 8.21 (d, J=10.6 Hz, 1H), 4.31 (s, 2H), 3.75-3.83 (m, 3H), 3.10-3.25 (m, 4H), 2.95-3.04 (m, 2H), 2.90 (s, 3H), 2.05-2.30 (m, 5H), 1.63-1.71 (m, 1H); MS (ESI) m/z 545.3 (M+H).

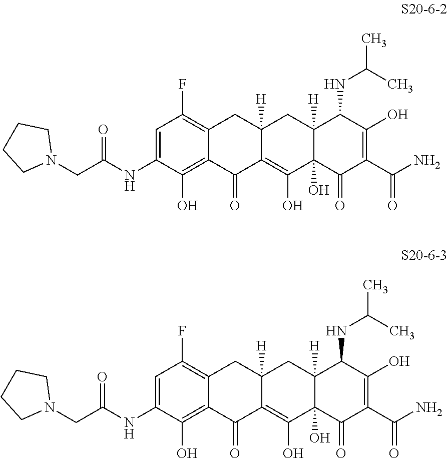 [SMILES: CC(C)N[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12 ; CC(C)N[C@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compounds S20-6-2 and S20-6-3 were prepared from compound S20-3 by using General Procedures B-1 with acetone, C, and D-1. S20-6-2: ¹H NMR (400 MHz, CD₃OD, dihydrochloride salt) δ 8.21 (d, J=10.6 Hz, 1H), 4.30 (s, 2H), 3.75-3.85 (m, 3H), 3.10-3.25 (m, 3H), 2.95-3.04 (m, 1H), 2.80-2.85 (m, 1H), 2.05-2.27 (m, 5H), 1.80-1.90 (m, 2H), 1.53-1.62 (m, 1H), 1.35-1.45 (m, 6H); MS (ESI) m/z 573.3 (M+H). S20-6-3: ¹H NMR (400 MHz, CD₃OD, dihydrochloride salt) δ 8.21 (d, J=10.6 Hz, 1H), 4.30 (s, 2H), 3.75-3.82 (m, 3H), 3.63-3.70 (m, 1H), 3.08-3.22 (m, 3H), 2.81-2.98 (m, 2H), 2.05-2.21 (m, 7H), 1.40-1.46 (m, 6H); MS (ESI) m/z 573.3 (M+H).

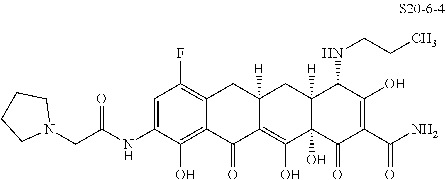 [SMILES: CCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-4 was prepared from compound S20-3 by using General Procedures B-1 with propionaldehyde, C, and D-1. ¹H NMR (400 MHz, CD₃OD, dihydrochloride salt) δ 8.20 (d, J=10.6 Hz, 1H), 4.30 (s, 2H), 3.72-3.81 (m, 3H), 3.10-3.25 (m, 3H), 2.95-3.04 (m, 2H), 2.80-2.87 (m, 2H), 2.05-2.25 (m, 6H), 1.80-1.90 (m, 2H), 1.55-1.60 (m, 1H), 0.98-1.05 (t, J=7.8 Hz, 3H); MS (ESI) m/z 573.2 (M+H).

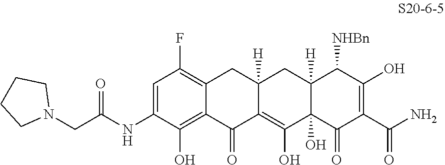

Compound S20-6-5 was prepared from compound S20-3 by using General Procedures B-1 with benzaldehyde, C, and D-1. ¹H NMR (400 MHz, CD₃OD, dihydrochloride salt) δ 8.21 (d, J=10.6 Hz, 1H), 7.56-7.60 (m, 2H), 7.45-7.51 (m, 3H), 4.46-4.51 (m, 1H), 4.31 (s, 2H), 3.72-3.83 (m, 5H), 2.90-3.20 (m, 3H), 1.97-2.25 (m, 7H), 1.25-1.30 (m, 1H); MS (ESI) m/z 621.2 (M+H).

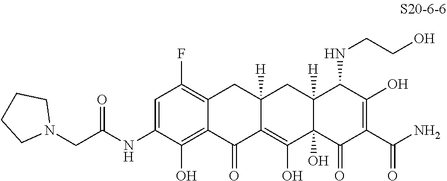 [SMILES: NC(=O)C1=C(O)[C@@H](NCCO)[C@@H]2C[C@@H]3Cc4c(F)cc(NC(=O)CN5CCCC5)c(O)c4C(=O)C3=C(O)[C@]2(O)C1=O]

Compound S20-6-6 was prepared from compound S20-3 by using General Procedures B-1 with 2-((tert-butyldimethylsilyl)oxy)acetaldehyde, C, and D-1. ¹H NMR (400 MHz, CD₃OD, dihydrochloride salt) δ 8.20 (d, J=10.6 Hz, 1H), 4.32 (s, 2H), 3.75-3.95 (m, 5H), 3.40-3.45 (m, 1H), 2.95-3.25 (m, 5H), 2.80-2.90 (m, 1H), 2.03-2.30 (m, 6H), 1.53-1.62 (m, 1H); MS (ESI) m/z 575.2 (M+H).

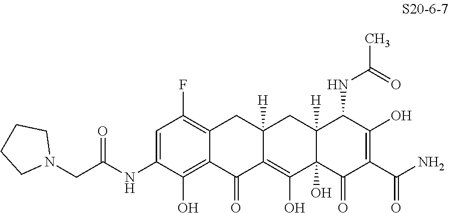 [SMILES: CC(=O)N[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-7 was prepared from compound S20-3 by using General Procedures B-2 with Ac₂O, C, and D-1. ¹H NMR (400 MHz, CD₃OD, hydrochloride salt) δ 8.20 (d, J=10.6 Hz, 1H), 4.69-4.72 (m, 1H), 4.41 (s, 2H), 3.75-3.81 (m, 2H), 3.15-3.21 (m, 3H), 2.90-3.10 (m, 2H), 2.30-2.45 (m, 3H), 2.05-2.20 (m, 3H), 2.01 (s, 3H), 1.55-1.63 (m, 1H); MS (ESI) m/z 573.2 (M+H).

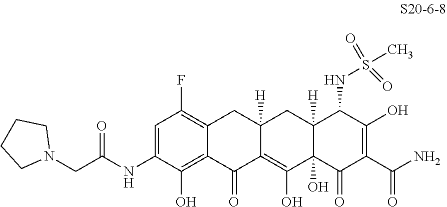 [SMILES: CS(=O)(=O)N[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-8 was prepared from compound S20-3 by using General Procedures B-2 with Ms₂O, C, and D-1. ¹H NMR (400 MHz, CD₃OD, hydrochloride salt) δ 8.20 (d, J=10.6 Hz, 1H), 4.41 (s, 2H), 4.08-4.11 (m, 1H), 3.75-3.82 (m, 3H), 3.09-3.21 (m, 4H), 2.95-3.03 (m, 1H), 2.55-2.61 (m, 3H), 2.02-2.30 (m, 5H), 1.66-1.72 (m, 1H); MS (ESI) m/z 609.2 (M+H).

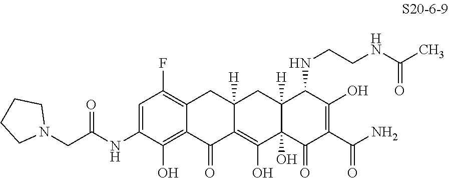 [SMILES: CC(=O)NCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-9 was prepared from compound S20-3 by using General Procedures B-1 with N-Boc-2-aminoacetaldehyde, treatment with HCl (4N aqueous) in dioxane, B-2 with Ac₂O, C, and D-1. ¹H NMR (400 MHz, CD₃OD, dihydrochloride salt) δ 8.23 (d, J=11.0 Hz, 1H), 4.31 (s, 2H), 3.96 (s, 1H), 3.83-3.73 (m, 2H), 3.65-3.52 (m, 1H), 3.52-3.42 (m, 2H), 3.24-3.08 (m, 3H), 3.06-2.96 (m, 1H), 2.82-2.75 (m, 1H), 2.32-1.96 (m, 10H), 1.63-1.50 (m, 1H); MS (ESI) m/z 616.5 (M+H).

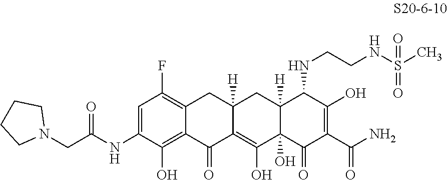 [SMILES: CS(=O)(=O)NCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-10 was prepared from compound S20-3 by using General Procedures B-1 with N-Boc-2-aminoacetaldehyde, treatment with HCl (4N aqueous) in dioxane, B-2 with Ms₂O, C, and D-1. ¹H NMR (400 MHz, CD₃OD, dihydrochloride salt) δ 8.23 (d, J=11.0 Hz, 1H), 4.47 (s, 2H), 4.30 (s, 2H), 4.02 (s, 1H), 3.83-3.71 (m, 2H), 3.54-3.43 (m, 3H), 3.28-3.11 (m, 4H), 3.12 (s, 3H), 3.00 (s, 3H), 2.87-2.79 (m, 1H), 2.32-2.00 (m, 5H), 1.63-1.50 (m, 1H); MS (ESI) m/z 652.3 (M+H).

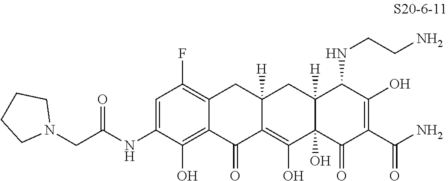 [SMILES: NCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-11 was prepared from compound S20-3 by using General Procedures B-1 with N-Boc-2-aminoacetaldehyde, C, and D-1. ¹H NMR (400 MHz, CD₃OD, trihydrochloride salt) δ 8.24 (d, J=11.0 Hz, 1H), 4.31 (s, 2H), 4.01 (s, 1H), 3.83-3.71 (m, 3H), 3.66-3.54 (m, 1H), 3.45-3.35 (m, 2H), 3.34-3.28 (m, 1H), 3.34-2.91 (m, 7H), 2.34-2.03 (m, 7H), 1.65-1.50 (m, 1H); MS (ESI) m/z 574.2 (M+H).

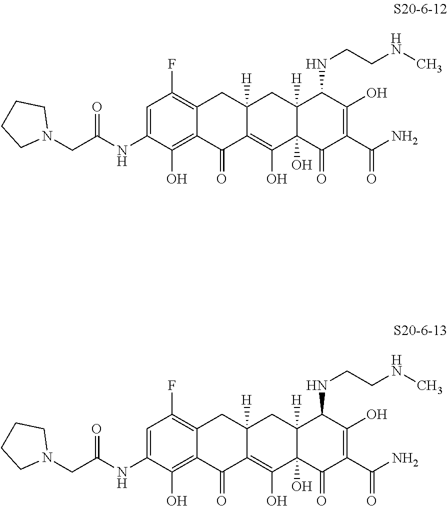 [SMILES: CNCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12 ; CNCCN[C@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compounds S20-6-12 and S20-6-13 were prepared from known compound S20-7 (prepared using literature procedure including WO 2014/036502 A2) by using General Procedure B-1 with N-Boc-2-methylaminoacetaldehyde, treatment with HCl (4N aqueous) in dioxane and purification via reverse phase preparative HPLC as described in General Procedure D-1. S20-6-12: ¹H NMR (400 MHz, CD₃OD, trihydrochloride salt) δ 8.23 (d, J=11.0 Hz, 1H), 4.31 (s, 2H), 4.03 (s, 1H), 3.86-3.72 (m, 3H), 3.72-3.61 (m, 1H), 3.46 (t, J=7.0 Hz, 1H), 3.24-3.09 (m, 5H), 2.79 (m, 3H), 2.32-2.01 (m, 6H), 1.63-1.50 (m, 1H); MS (ESI) m/z 588.4 (M+H). S20-6-13. ¹H NMR (400 MHz, CD₃OD, trihydrochloride salt) δ 8.22 (d, J=11.0 Hz, 1H), 4.83 (d, J=4.9 Hz, 1H), 4.31 (s, 2H), 3.87-3.72 (m, 3H), 3.70-3.59 (m, 1H), 3.57-3.42 (m, 2H), 3.24-3.09 (m, 3H), 309-3.01 (m, 1H), 3.01-2.89 (m, 1H), 2.82 (s, 3H), 2.36-1.99 (m, 6H), 1.56-1.43 (m, 1H); MS (ESI) m/z 588.4 (M+H).

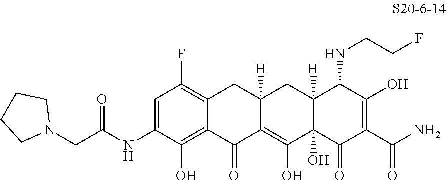 [SMILES: NC(=O)C1=C(O)[C@@H](NCCF)[C@@H]2C[C@@H]3Cc4c(F)cc(NC(=O)CN5CCCC5)c(O)c4C(=O)C3=C(O)[C@]2(O)C1=O]

Compound S20-6-14 was prepared from compound S20-3 by using General Procedures B-1 with FCH₂CHO (prepared from the corresponding alcohol according to the literature procedure in WO 2011146089 A1), C, and D-1. ¹H NMR (400 MHz, CD₃OD, dihydrochloride salt) δ 8.23 (d, J=11.0 Hz, 1H), 4.88-4.72 (m, 2H), 4.32 (s, 2H), 4.00 (s, 1H), 3.85-3.58 (m, 4H), 3.27-3.08 (m, 3H), 3.07-2.94 (m, 4H), 2.89 (d, J=13.4 Hz, 1H), 2.34-1.99 (m, 6H), 1.65-1.51 (m, 1H); MS (ESI) m/z 577.3 (M+H).

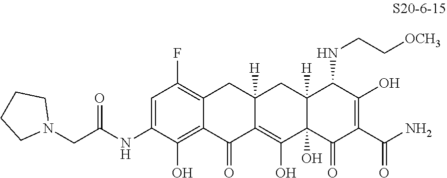 [SMILES: COCCN[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

Compound S20-6-15 was prepared from compound S20-3 by using General Procedures B-1 with CH₃OCH₂CHO (prepared from the corresponding alcohol per the literature procedure in WO 2011146089 A1), C, and D-1. ¹H NMR (400 MHz, CD₃OD, dihydrochloride salt) δ 8.23 (d, J=11.0 Hz, 1H), 4.31 (s, 2H), 3.96 (s, 1H), 3.83-3.62 (m, 4H), 3.54-3.44 (m, 2H), 3.40 (s, 3H), 3.24-3.09 (m, 3H), 3.05-2.93 (m, 1H), 2.85 (ad, J=12.8 Hz, 1H), 2.33-2.00 (m, 6H), 1.65-1.52 (m, 1H).

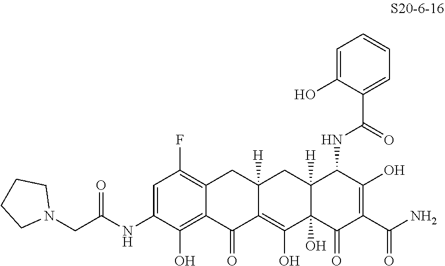 [SMILES: NC(=O)C1=C(O)[C@@H](NC(=O)c2ccccc2O)[C@@H]2C[C@@H]3Cc4c(F)cc(NC(=O)CN5CCCC5)c(O)c4C(=O)C3=C(O)[C@]2(O)C1=O]

A flask was charged with S20-7 (51 mg, 0.096 mmol, 1 eq, (prepared using literature procedure including WO 2014/036502 A2), N-(3-dimethylaminopropyl)-N′-ethylcarbodiimide hydrochloride (29.8 mg, 0.16 mmol, 1.1 eq) and 1-hydroxybenzotriazole (19.7 mg, 0.15 mmol, 1.5 eq) and placed under N₂. To the vessel was added DMF (2 mL) and DIEA (26.6 μL, 0.15 mmol, 1.6 eq). The mixture was stirred at room temperature for 1 h, then purified by preparative reverse phase HPLC on a Waters Autopurification system using a Phenomenex Polymerx 10μ RP-γ 100A column [10 μm, 150×21.20 mm; flow rate, 20 mL/min; Solvent A: 0.05 N HCl/water; Solvent B: CH₃CN; injection volume: 2.0 mL (0.05 N HCl/water); gradient: 0→85% B in A over 30 min; mass-directed fraction collection]. Fractions containing the desired product were collected and freeze-dried to yield compound S20-6-16. ¹H NMR (400 MHz, CD₃OD, hydrochloride salt) δ 8.18 (d, J=11.0 Hz, 1H), 7.92 (dd, J=7.9, 1.8 Hz, 1H), 7.43-7.35 (m, 1H), 6.96-6.88 (m, 2H), 5.69-5.60 (m, 1H), 4.29 (s, 2H), 3.91-3.58 (m, 2H), 3.12-3.04 (m, 1H), 2.96-2.87 (m, 1H), 2.85-2.73 (m, 1H), 2.30-2.00 (m, 7H), 1.49-1.35 (m, 1H); MS (ESI) m/z 651.3 (M+H).

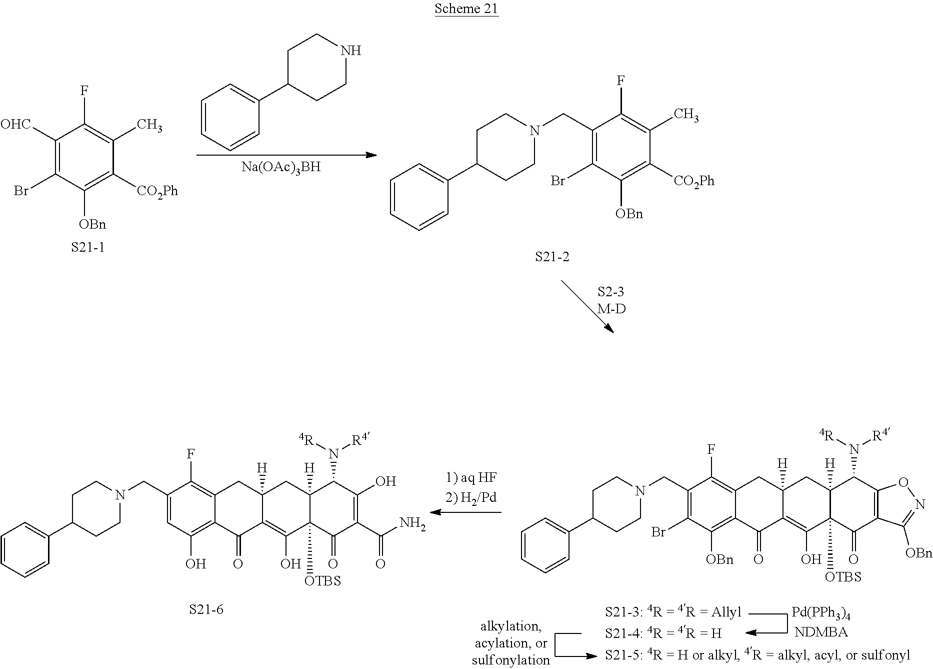

The following compounds were prepared per Scheme 21.

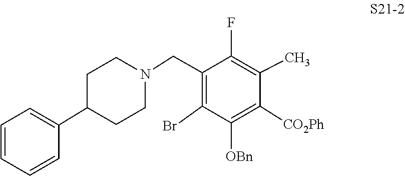

To a solution of S21-1 (1.65 g, 3.72 mmol, 1 eq, prepared per literature procedure: J Med. Chem., 2013, 56, 8112-8138) in DCM (37 mL) was added 4-phenylpiperidine (2.99 g, 18.6 mmol, 5 eq), followed by HOAc (1 mL, 18.6 mmol, 5 eq). After one hour, STAB (2.37 g, 11.18 mmol, 3 eq was added. After 1 h, the reaction mixture was diluted with EtOAc (150 mL) and washed with saturated, aqueous NaHCO₃ (2×90 mL), 1N NaOH (30 mL) and brine (30 mL). The organic layer was dried over Na₂SO₄, filtered, and concentrated under reduced pressure. The resulting residue was purified via flash column chromatography on silica gel with CH₃OH in DCM, 0.5%-3% to provide S21-2 (1.42 g, 65%). ¹H NMR (400 MHz, CDCl₃) δ 13.35 (brs, 1H), 7.53-7.45 (m, 2H), 7.43-7.18 (m, 11H), 7.09-7.02 (m, 2H), 5.14 (s, 2H), 4.60 (s, 2H), 3.85-3.75 (m, 2H), 2.92-2.55 (m, 4H), 2.42 (s, 3H), 2.04-1.94 (m, 2H). MS (ESI) m/z 588.37 (M+H).

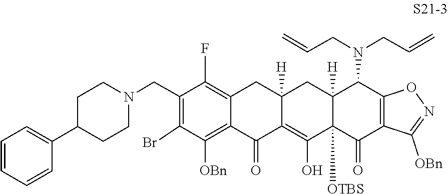 [SMILES: C=CCN(CC=C)[C@@H]1c2onc(OCc3ccccc3)c2C(=O)[C@@]2(O[Si](C)(C)C(C)(C)C)C(O)=C3C(=O)c4c(c(F)c(CN5CCC(c6ccccc6)CC5)c(Br)c4OCc4ccccc4)C[C@H]3C[C@@H]12]

Compound S21-3 was prepared from S21-2 (and S2-3 using General Procedure E. ¹H NMR (400 MHz, CDCl₃) δ 15.92 (s, 1H), 7.62-7.48 (m, 4H), 7.43-7.14 (m, 11H), 5.89-5.76 (m, 2H), 5.38 (s, 2H), 5.23 (d, J=17.1 Hz, 2H), 5.15 (d, J=9.58 Hz, 2H), 5.04-4.94 (m, 2H), 4.07 (d, J=10.4 Hz, 1H), 3.79 (brs, 1H), 3.39-3.30 (m, 2H), 3.28-3.14 (m, 3H), 3.13-2.98 (m, 2H), 5.67-2.42 (m, 4H), 2.39-2.25 (m, 1H), 2.15 (d, J=17.7 Hz, 1H), 1.8 (brs, 1H), 0.83 (s, 9H), 0.27 (s, 3H), 0.13 (s, 3H). MS (ESI) m/z 1028.69 (M+H).

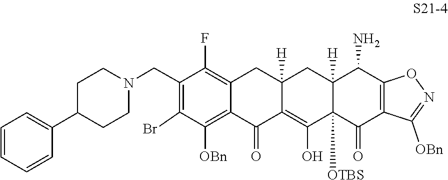 [SMILES: CC(C)(C)[Si](C)(C)O[C@@]12C(=O)c3c(OCc4ccccc4)noc3[C@@H](N)[C@@H]1C[C@@H]1Cc3c(F)c(CN4CCC(c5ccccc5)CC4)c(Br)c(OCc4ccccc4)c3C(=O)C1=C2O]

Compound S21-4 was prepared from compound S21-3 by using General Procedure A. ¹H NMR (400 MHz, CDCl₃, rotamers, all peaks are broadened) δ 16.19 (m, 1H), 13.19 (brs, 1H), 7.54-7.17 (m, 15H), 5.42-4.91 (m, 5H), 4.61-4.35 (m, 2H), 4.09-3.99 (m, 1H), 3.90-3.60 (m, 2H), 3.29-2.44 (m, 7H), 2.36-1.82 (m, 4H), 0.87-0.59 (m, 9H), 0.22-−0.04 (m, 6H). MS (ESI) m/z 948.60 (M+H).

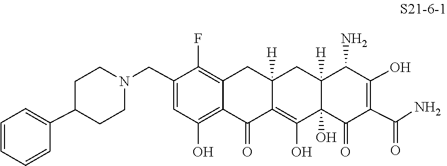 [SMILES: NC(=O)C1=C(O)[C@@H](N)[C@@H]2C[C@@H]3Cc4c(F)c(CN5CCC(c6ccccc6)CC5)cc(O)c4C(=O)C3=C(O)[C@]2(O)C1=O]

Compound S21-6-1 was prepared from compound S21-4 by using General Procedures C and D-2. ¹H NMR (400 MHz, CD₃OD, dihydrochloride salt) δ 7.35-7.18 (m, 5H), 7.09 (d, J=6.4 Hz, 1H), 4.40 (s, 2H), 3.87 (s, 1H), 3.69-3.56 (m, 2H), 3.28-3.17 (m, 3H), 3.07-2.95 (m, 1H), 2.94-2.83 (m, 1H), 2.63 (d, J=12.8 Hz, 1H), 2.43-2.31 (m, 1H), 2.28-2.20 (m, 1H), 2.15-1.91 (m, 4H), 1.67-1.54 (m, 1H); MS (ESI) m/z 578.46 (M+H).

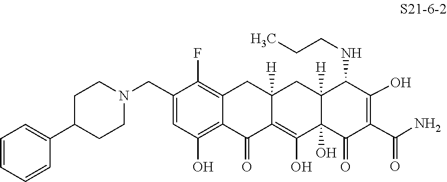 [SMILES: CCCN[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-2 was prepared from compound S21-3 by using General Procedures C and D-2. ¹H NMR (400 MHz, CD₃OD, dihydrochloride salt) δ 7.36-7.19 (m, 5H), 7.13-7.07 (m, 1H), 4.42 (s, 2H), 3.86 (s, 1H), 3.69-3.59 (m, 2H), 3.28-3.14 (m, 5H), 3.09-2.96 (m, 1H), 2.93-2.81 (m, 2H), 2.41-2.31 (m, 1H), 2.26-2.18 (m, 1H), 2.15-1.91 (m, 4H), 1.83-1.70 (m, 2H), 1.65-1.53 (m, 1H), 1.03 (t, J=8 Hz, 3H). MS (ESI) m/z 620.50 (M+H).

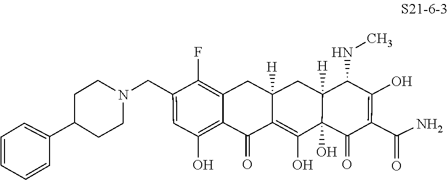 [SMILES: CN[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

To a solution of S21-4 in THF was added allylbromide (4 eq), potassium carbonate (8 eq) and a catalytic amount of NaI. This mixture was headed at 40° C. for 5 h. The solution was diluted with brine and extracted with EtOAc. The organic layers were concentrated under reduced pressure and the resulting residue was purified by flash column chromatography on silica gel with 10%-80% EtOAc in hexanes. The resulting product was subjected to General Procedures B-1, C and D-2 to provide S21-6-3: ¹H NMR (400 MHz, CD₃OD, dihydrochloride salt) δ 7.38-7.20 (m, 5H), 7.15-7.07 (m, 1H), 4.42 (s, 2H), 3.80 (s, 1H), 3.70-3.59 (m, 2H), 3.28-3.15 (m, 3H), 3009-2.98 (m, 1H), 2.95-2.73 (m, 5H), 2.42-2.30 (m, 1H), 2.26-2.17 (m, 1H), 2.16-1.91 (m, 4H), 1.67-1.54 (m, 1H); MS (ESI) m/z 592.4 (M+H).

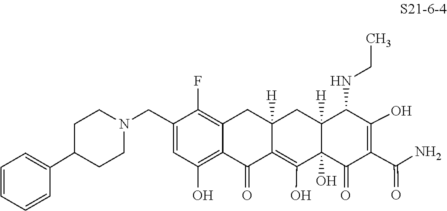 [SMILES: CCN[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-4 was prepared from compound S21-4 with CH₃CHO by using General Procedures B-1 (at 0° C.), C, and D2: ¹H NMR (400 MHz, CD₃OD dihydrochloride salt) δ 7.39-7.19 (m, 5H), 7.13-7.06 (m, 1H), 4.41 (s, 2H), 3.85 (s, 1H), 3.70-3.60 (m, 2H), 3.44-3.14 (m, 3H), 3.07-2.98 (m, 1H), 2.95-2.71 (m, 4H), 2.41-2.30 (m, 1H), 2.26-2.18 (m, 1H), 2.16-1.89 (m, 4H), 1.64-1.51 (m, 1H), 1.35 (t, J=7.3 Hz, 3H); MS (ESI) m/z 606.47 (M+H).

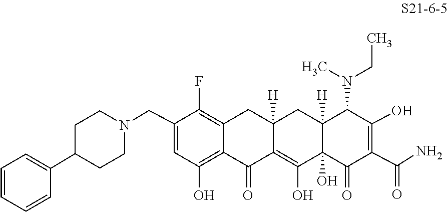 [SMILES: CCN(C)[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-5 was prepared from compound S21-4 with CH₃CHO by using General Procedures B-1 (at 0° C.), B-1 with HCHO, C, and D-2: ¹H NMR (400 MHz, CD₃OD, dihydrochloride salt) δ 7.38-7.17 (m, 5H), 7.14-7.09 (m, 1H), 4.42 (s, 2H), 4.22 (s, 0.5H), 4.13 (s, 0.5H), 3.71-3.60 (m, 2H), 3.54-3.40 (m, 1H), 3.29-3.17 (m, 2H), 3.16-2.83 (m, 6H), 2.41-2.30 (m, 1H), 2.30-2.20 (m, 1H), 2.15-1.94 (m, 4H), 1.73-1.59 (m, 1H), 1.46-1.33 (m, 3H); MS (ESI) m/z 620.50 (M+H).

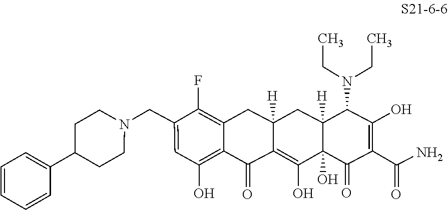 [SMILES: CCN(CC)[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-6 was prepared from compound S21-4 with CH₃CHO by using General Procedures B-1 (at 0° C.), B-1 again with CH₃CHO, C, and D-2: ¹H NMR (400 MHz, CD₃OD, dihydrochloride salt) δ 7.38-7.18 (m, 5H), 7.13-7.08 (m, 1H), 4.42 (s, 2H), 4.24 (s, 1H), 3.70-3.53 (m, 3H), 3.50-3.40 (m, 2H), 3.29-3.17 (m, 4H), 3.14-3.02 (m, 1H), 2.95-2.84 (m, 2H), 2.41-2.30 (m, 1H), 2.28-2.20 (m, 1H), 2.15-1.92 (m, 4H), 1.72-1.58 (m, 1H), 1.40 (t, J=7.2 Hz, 6H); MS (ESI) m/z 634.49 (M+H).

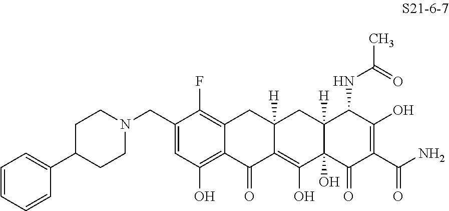 [SMILES: CC(=O)N[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-7 was prepared from compound S21-4 by using General Procedures B-2 with Ac₂O, C, and D-2: ¹H NMR (400 MHz, CD₃OD, dihydrochloride salt) δ 8.39-8.31 (m, 1H), 7.37-7.19 (m, 5H), 7.09-7.03 (m, 1H), 4.76-4.69 (m, 1H), 4.42 (s, 2H), 3.71-3.61 (m, 2H), 3.28-3.21 (m, 1H), 3.19-3.11 (m, 1H), 3.08-2.98 (m, 1H), 2.95-2.84 (m, 1H), 2.65-2.53 (m, 1H), 2.47-2.34 (m, 2H), 2.28-2.20 (m, 1H), 2.18-1.91 (m, 7H), 1.68-1.58 (m, 1H); MS (ESI) m/z 620.3 (M+H).

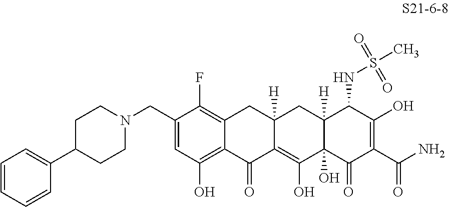 [SMILES: CS(=O)(=O)N[C@@H]1C(O)=C(C(N)=O)C(=O)[C@@]2(O)C(O)=C3C(=O)c4c(O)cc(CN5CCC(c6ccccc6)CC5)c(F)c4C[C@H]3C[C@@H]12]

Compound S21-6-8 was prepared from compound S21-4 by using General Procedures B-2 with Ms₂O, C, and D-2: ¹H NMR (400 MHz, CD₃OD, dihydrochloride salt) δ 7.35-7.17 (m, 5H), 7.03 (d, J=5.6 Hz, 1H), 4.37 (s, 2H), 4.14-4.09 (m, 1H), 3.66-3.55 (m, 2H), 3.27-3.09 (m, 6H), 3.08-2.98 (m, 1H), 2.92-2.82 (m, 1H), 2.67-2.54 (m, 1H), 2.53-2.44 (m, 1H), 2.37-2.26 (m, 1H), 2.13-2.88 (m, 4H), 1.79-1.69 (m, 1H); MS (ESI) m/z 656.3 (M+H).

### Example 4: Antibacterial Activity

The antibacterial activities for the compounds of the invention were studied according to the following protocols.

### Minimum Inhibitory Concentration (MIC) Assay

MICs were determined according to the Clinical and Laboratory Standards Institute (CLSI) guidances (e.g., CLSI. Performance standards for antimicrobial susceptibility testing; nineteenth information supplement. CLSI document M100-S19, CLSI, 940 West Valley Road, Suite 1400, Wayne, Pa. 19087-1898, USA, 2009). Briefly, frozen bacterial strains were thawed and subcultured onto Mueller Hinton Broth (MHB) or other appropriate media (Streptococcus requires blood and Haemophilus requires hemin and NAD). Following incubation overnight, the strains were subcultured onto Mueller Hinton Agar and again incubated overnight. Colonies were observed for appropriate colony morphology and lack of contamination. Isolated colonies were selected to prepare a starting inoculum equivalent to a 0.5 McFarland standard. The starting inoculum was diluted 1:125 (this is the working inoculum) using MHB for further use. Test compounds were prepared by dilution in sterile water to a final concentration of 5.128 mg/mL. Antibiotics (stored frozen, thawed and used within 3 hours of thawing) and compounds were further diluted to the desired working concentrations.

The assays were run as follows. Fifty μL of MHB was added to wells 2-12 of a 96-well plate. One hundred μL of appropriately diluted antibiotics was added to well 1. Fifty μL of antibiotics was removed from well 1 and added to well 2 and the contents of well 2 mixed by pipetting up and down five times. Fifty μL of the mixture in well 2 was removed and added to well 3 and mixed as above. Serial dilutions were continued in the same manner through well 12. Fifty μL was removed from well 12 so that all contained 50 μL. Fifty μL of the working inoculum was then added to all test wells. A growth control well was prepared by adding 50 μL of working inoculum and 50 μL of MHB to an empty well. The plates were then incubated at 37° C. overnight, removed from the incubator and each well was read on a plate reading mirror. The lowest concentration (MIC) of test compound that inhibited the growth of the bacteria was recorded.

### Example

| | 1 | 2 | 3 | 4 | 5 | 6 | 7 | 8 | 9 | 10 | 11 | 12 |
|---|---|---|---|---|---|---|---|---|---|---|---|---|
| [Abt] | 32 | 16 | 8 | 4 | 2 | 1 | 0.5 | 0.25 | 0.125 | 0.06 | 0.03 | 0.015 |
| Growth | − | − | − | − | − | + | + | + | + | + | + | + |

[abt] = antibiotic concentration in the well in μg/ml
Growth = bacterial growth (cloudiness)
Interpretation: MIC = 2 μg/mL

Protocol for Determining Inoculum Concentration (Viable Count) Fifty 50 μl of the inoculum was pipetted into well 1. Ninety μl of sterile 0.9% NaCl was pipetted into wells 2-6 of a 96-well microtiter plate. Ten μL from was removed from well 1 and added it to well 2 followed by mixing. Ten μL was removed from well two and mixed with the contents of well 3 and so on creating serial dilutions through well 6. Ten μL was removed from each well and spotted onto an appropriate agar plate. The plate was placed into an incubator overnight. The colonies in spots that contain distinct colonies were counted. Viable count was calculated by multiplying the number of colonies by the dilution factor.

| Spot from Well | 1 | 2 | 3 | 4 | 5 | 6 |
|---|---|---|---|---|---|---|
| Dilution | 10² | 10³ | 10⁴ | 10⁵ | 10⁶ | 10⁷ |
| Factor | | | | | | |

### Bacterial Strains

The following bacterial strains, listed below, were examined in minimum inhibitory concentration (MIC) assays.

| | STRAIN | |
|---|---|---|
| ORGANISM | DESIGNATION | KEY PROPERTIES |
| Staphylococcus aureus | SA100 | ATCC 13709, MSSA, Smith strain |
| Staphylococcus aureus | SA101 | ATCC 29213, CLSI quality control |
| | | strain, MSSA |
| Staphylococcus aureus | SA191 | HA-MRSA, tetracycline-resistant, |
| | | lung infection model isolate |
| Staphylococcus aureus | SA161 | HA-MRSA, tetracycline-resistant, |
| | | tet(M) |
| Staphylococcus aureus | SA158 | Tetracycline-resistant tet(K) |
| aaaureusaureus | | |
| Staphylococcus epidermidis | SE164 | ATCC 12228, CLSI quality control |
| | | strain, tetracycline-resistant |
| Enterococcus faecalis | EF103 | ATCC 29212, tet-I/R, control strain |
| Enterococcus faecalis | EF159 | Tetracycline-resistant, tet(M) |
| Enterococcus faecalis | EF327 | Wound isolate (US) tet(M) |
| Enterococcus faecium | EF404 | Blood isolate (US) tet(M) |
| Streptococcus pneumoniae | SP106 | ATCC 49619, CLSI quality control |
| | | strain |
| Streptococcus pneumoniae | SP160 | Tetracycline-resistant, tet(M) |
| Streptococcus pyogenes | SP312 | 2009 clinical isolate, tet(M) |
| Streptococcus pyogenes | SP193 | S. pyogenes for efficacy models; |
| | | tetS; sensitive to sulfonamides |
| Haemophilus influenzae | HI262 | Tetracycline-resistant, ampicillin- |
| | | resistant |
| Moraxella catarrhalis | MC205 | ATCC 8176, CLSI quality control |
| | | strain |
| Escherichia coli | EC107 | ATCC 25922, CLSI quality control |
| | | strain |
| Escherichia coli | EC155 | Tetracycline-resistant, tet(A) |
| Enterobacter cloacae | EC108 | ATCC 13047, wt |
| Enterobacter cloacae | EC603 | Urine isolate (Spain) |
| Escherichia coli | EC878 | MG1655 tolC::kan |
| Klebsiella pneumoniae | KP109 | ATCC 13883, wt |
| Klebsiella pneumoniae | KP153 | Tetracycline-resistant, tet(A), |
| | | MDR, ESBL⁺ |
| Klebsiella pneumoniae | KP457 | 2009 ESBL⁺, CTX-M, OXA |
| Proteus mirabilis | PM112 | ATCC 35659 |
| Proteus mirabilis | PM385 | Urine ESBL+ isolate |
| Pseudomonas aeruginosa | PA111 | ATCC 27853, wt, control strain |
| Pseudomonas aeruginosa | PA169 | Wt, parent of PA170-173 |
| Pseudomonas aeruginosa | PA173 | PA170 ΔmexX; MexXY-(missing a |
| | | functional efflux pump) |
| Pseudomonas aeruginosa | PA555 | ATCC BAA-47, wild type strain |
| | | PAO1 |
| Pseudomonas aeruginosa | PA556 | Multiple-Mex efflux pump knockout |
| | | strain |
| Pseudomonas aeruginosa | PA673 | 2009 urine isolate from catheter in |
| | | male from East North Central US |
| Pseudomonas aeruginosa | PA669 | 2009 clinical isolate from tracheal |
| | | aspirate |
| Pseudomonas aeruginosa | PA693 | 2009 isolate from corneal scraping |
| | | of female from Pacific US |
| Pseudomonas aeruginosa | PA1145 | Strain used in murine pneumonia |
| | | model |
| Acinetobacter baumannii | AB110 | ATCC 19606, wt |
| Acinetobacter baumannii | AB250 | Cystic fibrosis isolate, MDR |
| Stenotrophomonas maltophilia | SM256 | Cystic fibrosis isolate, MDR |
| Burkholderia cenocepacia | BC240 | Cystic fibrosis isolate, MDR |

*MDR, multidrug-resistant; MRSA, methicillin-resistant S. aureus; MSSA, methicillin-sensitive S. aureus; HA-MRSA, hospital-associated MRSA; tet(K), major gram-positive tetracycline efflux mechanism; tet(M), major gram-positive tetracycline ribosome-protection mechanism; ESBL⁺, extended spectrum β-lactamase

### Results

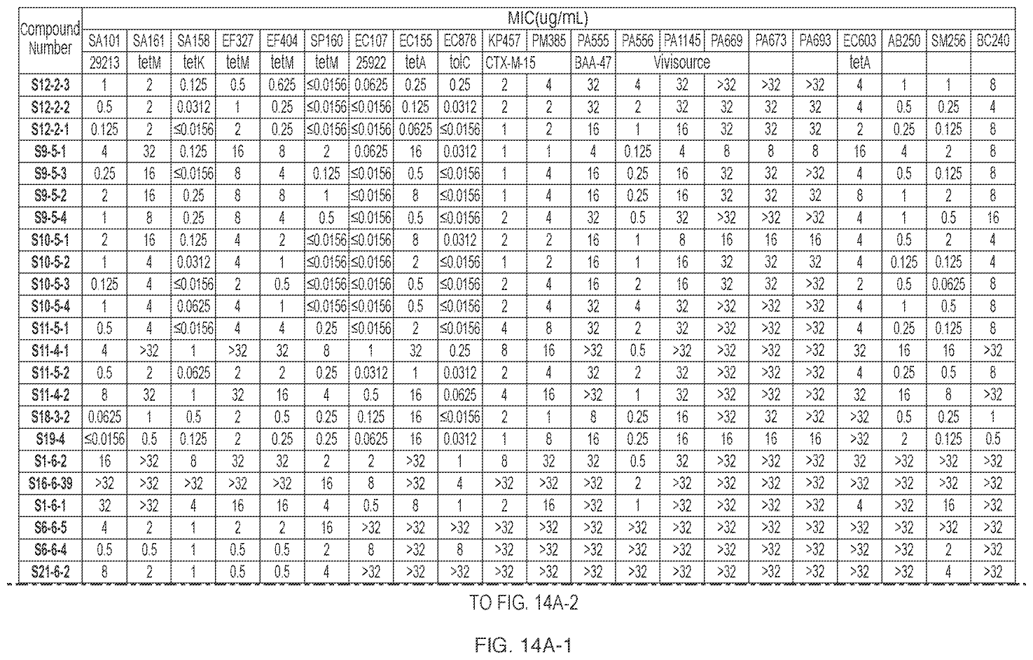

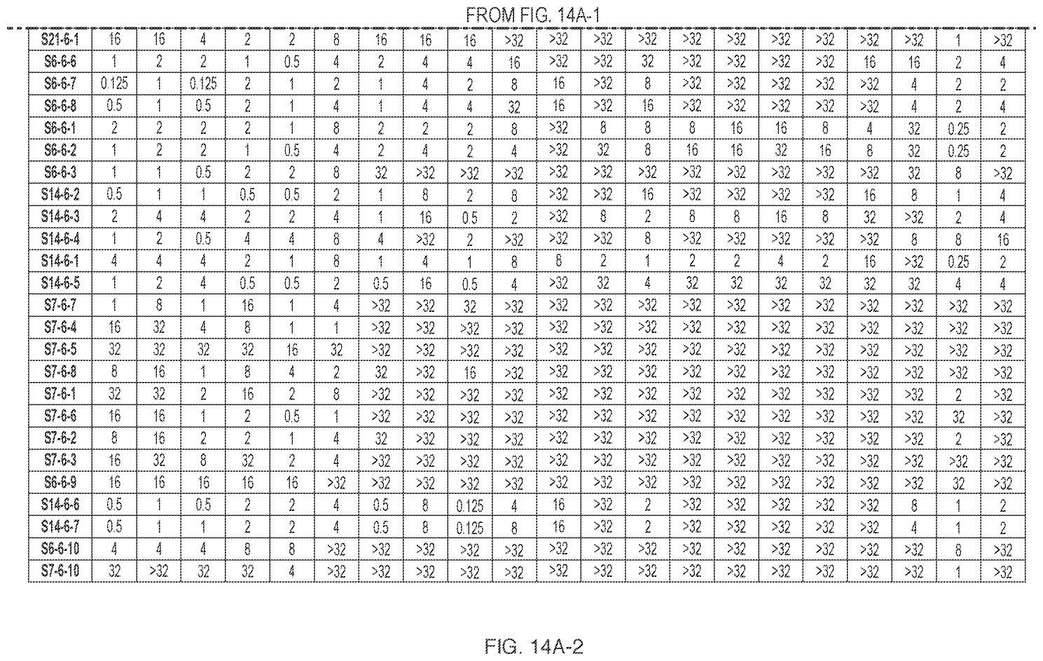

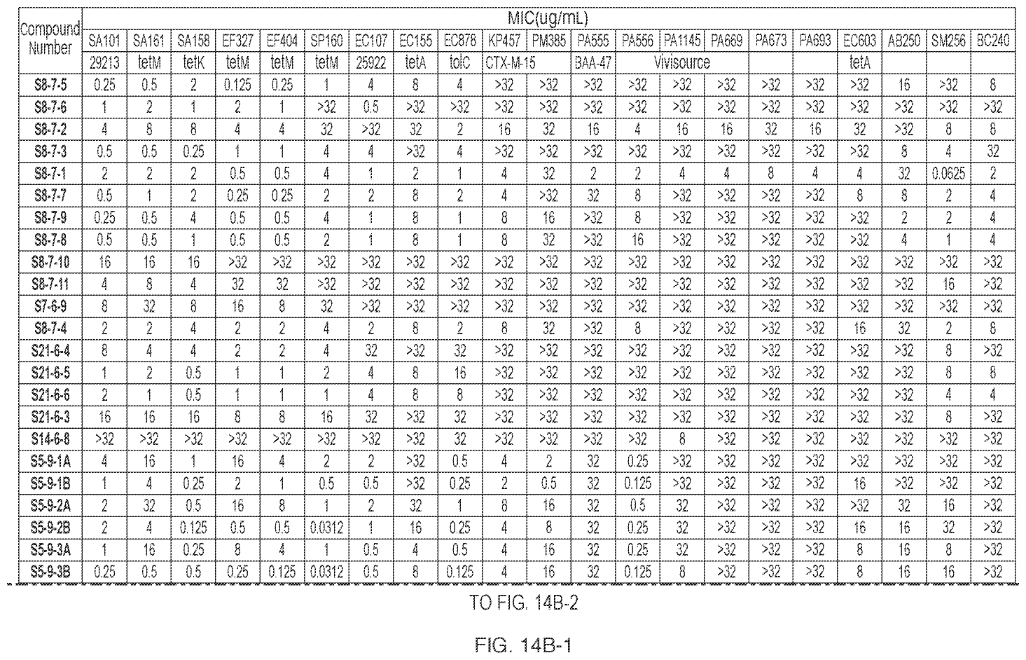

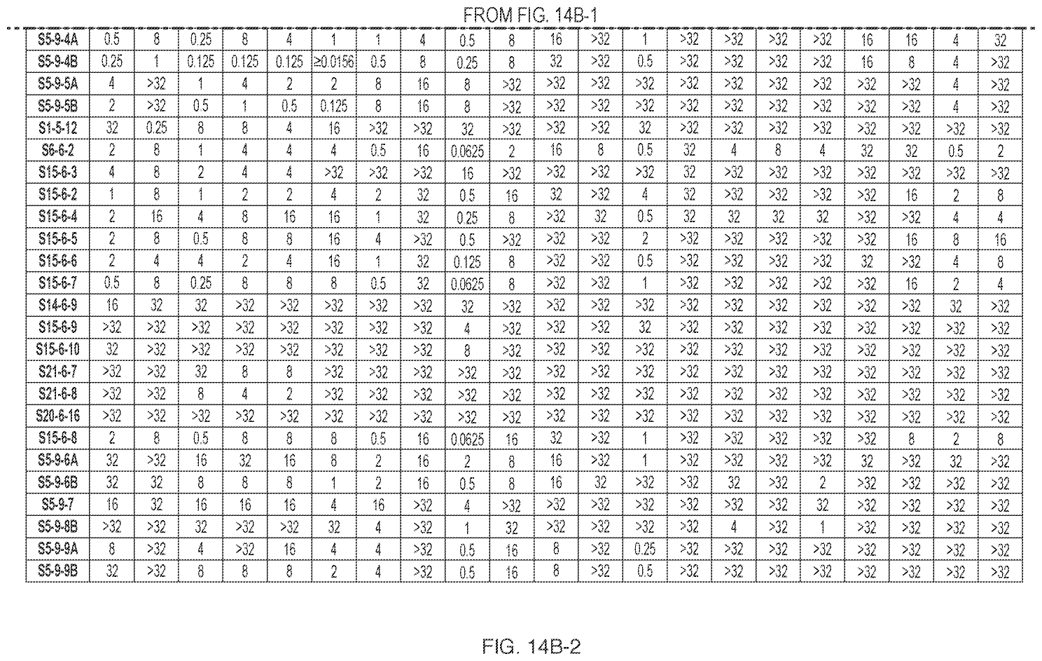

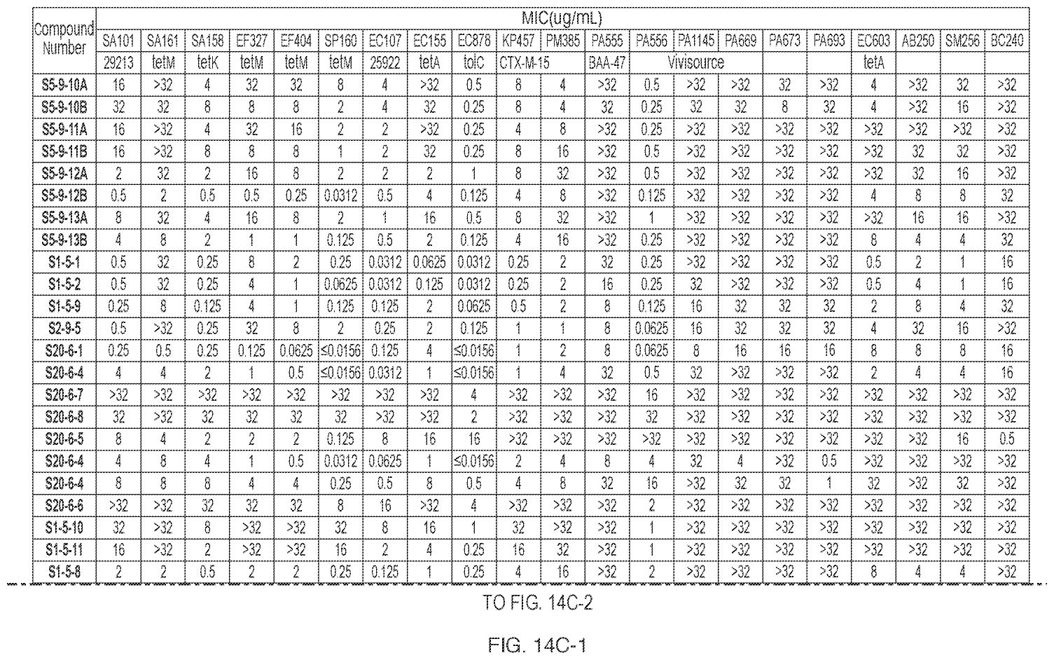

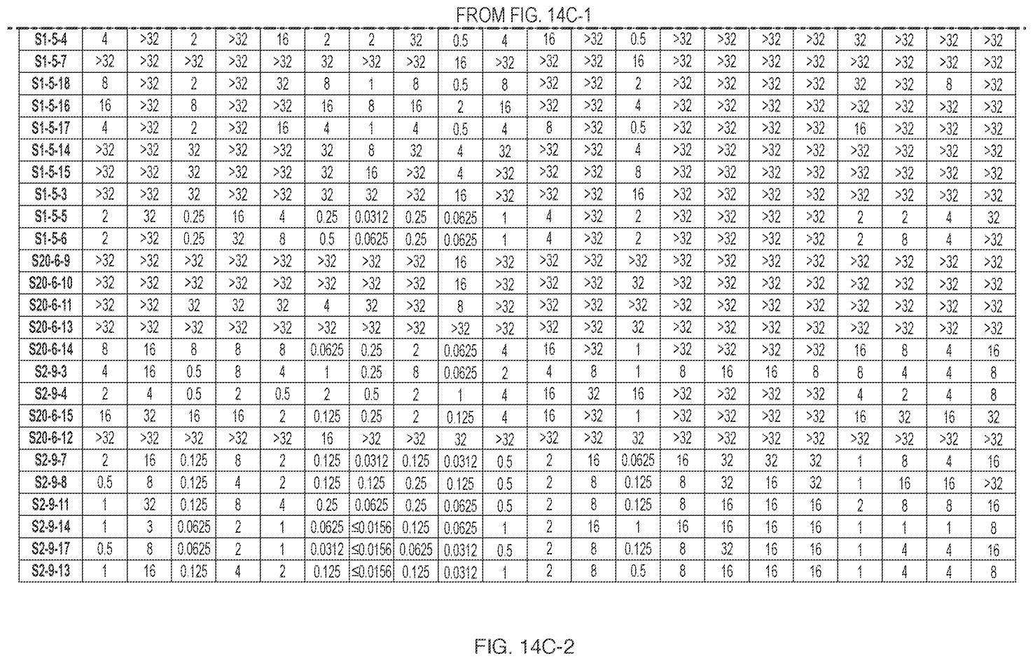

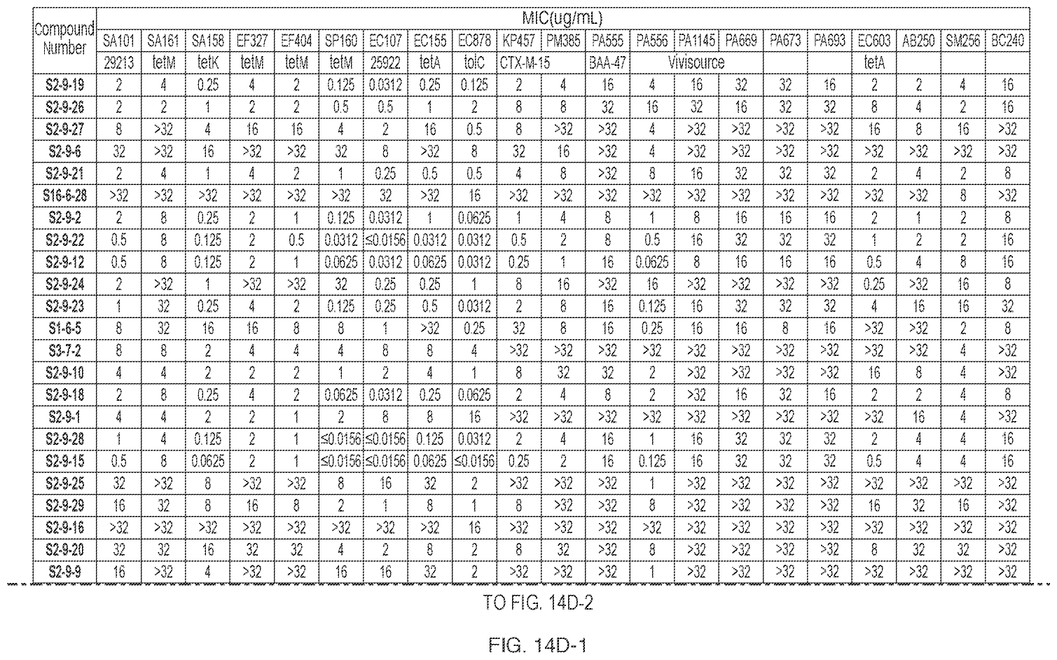

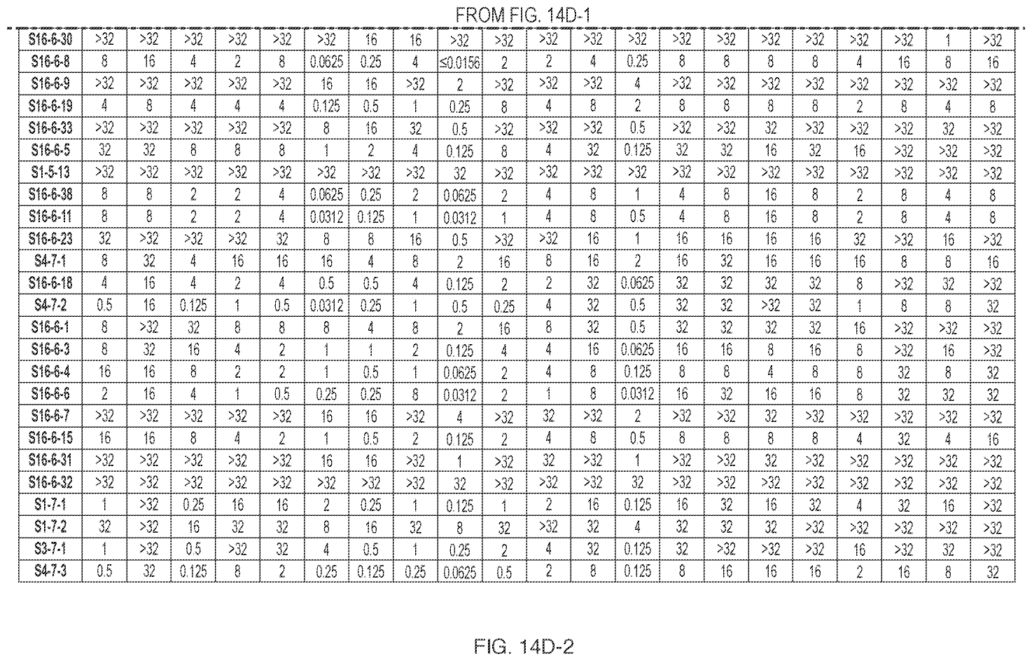

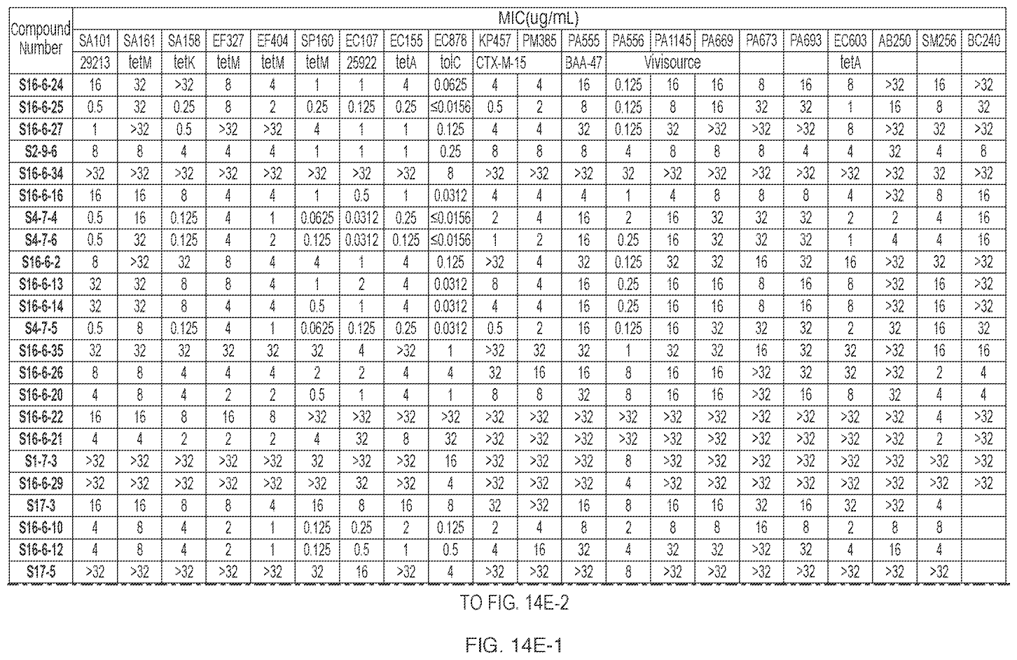

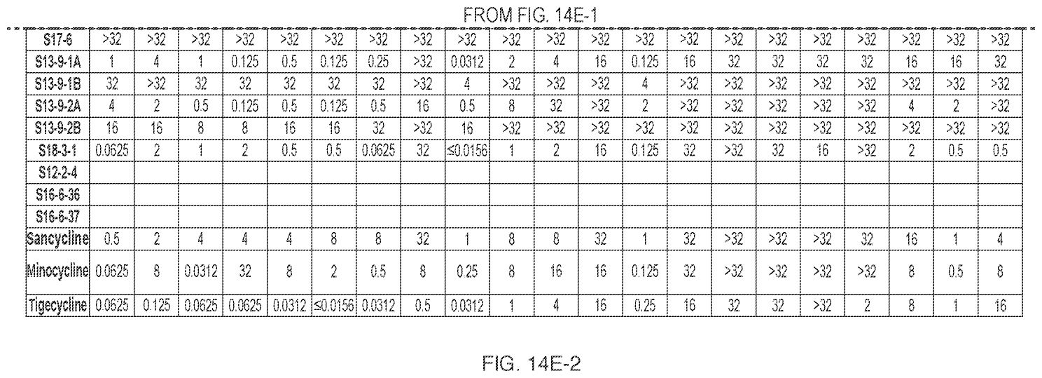

Values of minimum inhibition concentration (MIC) for tested compounds of the invention are provided in the table represented in FIG. 14A through FIG. 14E, collectively. MIC values are reported in μg/mL.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1-51. (canceled)

52. A method of treating a hematological cancer comprising administering to a subject in need of treatment an effective amount of a compound represented by the following structural formula 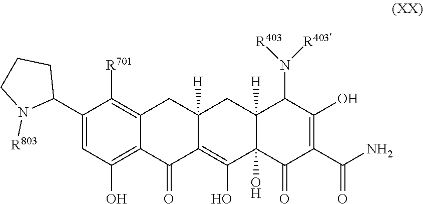
or a pharmaceutically acceptable salt thereof, or a pharmaceutically acceptable composition thereof, wherein:
R⁸⁰³ is H, a C₁₋₆ alkyl, a C₁₋₆ haloalkyl, C₁₋₆ hydroxyalkyl, a C₃₋₁₂ carbocyclyl-(C₀₋₃)alkylenyl, an amino-(C₁-C₄) alkyl, a mono- or di-(C₁-C₄ alkyl)amino-(C₁₋₄)alkyl, or a (4-13 member)heterocyclyl-(C₀-C₃)alkylenyl, wherein the heterocyclyl portion is optionally substituted with a C₁₋₃ alkyl;
R⁷⁰¹ is H, a C₁₋₄ alkyloxy, —OH, C₁₋₄ alkyl, a C₁₋₄ haloalkyl, or C₁₋₄ hydroxyalkyl, C₁₋₄ haloalkoxy; and
R⁴⁰³ and R^{403′}, each independently, is H; a C₁₋₄ alkyl; a C₁-C₄ haloalkyl; a C₃₋₁₂ carbocyclyl-(C₀-C₃)alkylenyl-, wherein the carbocyclyl portion is optionally substituted with a hydroxyl group; or a H₂NC(O)—(C₁-C₃)alkylenyl.

53-132. (canceled)

133. The method according to claim 52, wherein the compound is selected from: 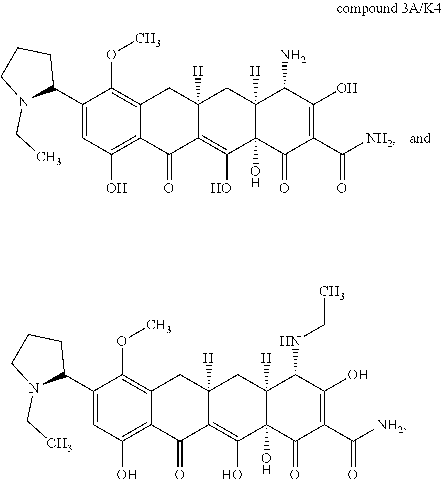
or a pharmaceutically acceptable salt thereof.

134. The method according to claim 52, wherein R⁷⁰¹ is —OCH₃, and R⁸⁰³ is ethyl.

135. The method according to claim 52, wherein R⁷⁰¹ is —OCH₃, and R⁴⁰³ and R^{403′} each is hydrogen.

136. The method according to claim 52, wherein R⁸⁰³ is ethyl and R⁴⁰³ and R^{403′} each is hydrogen.

137. The method according to claim 52, wherein R⁷⁰¹ is a —OCF₃, and R⁸⁰³ is methyl.

138. The method according to claim 52, wherein R⁷⁰¹ is —CF₃, and R⁸⁰³ is a C₁₋₄ alkyl or a (C₃-C₆)carbocyclyl-(C₀-C₃)alkylenyl.

139. The method according to claim 52, wherein the compound is represented by any one of the following structural formulas, or a pharmaceutically acceptable salt thereof:

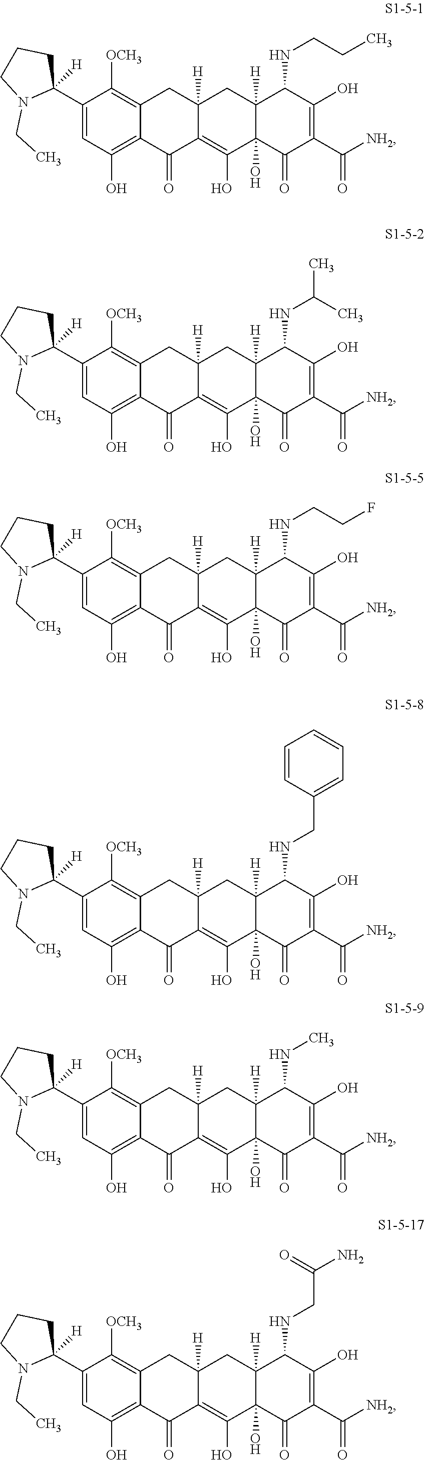

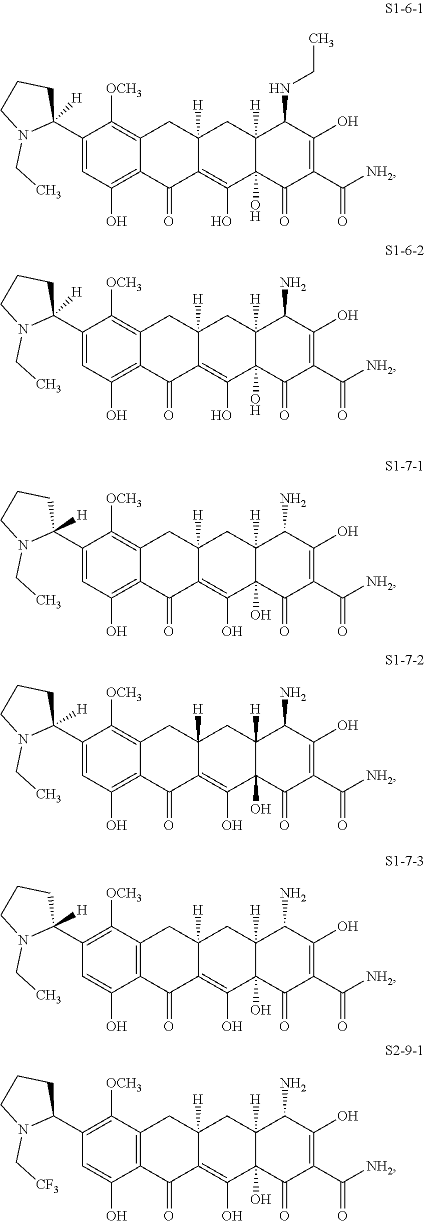

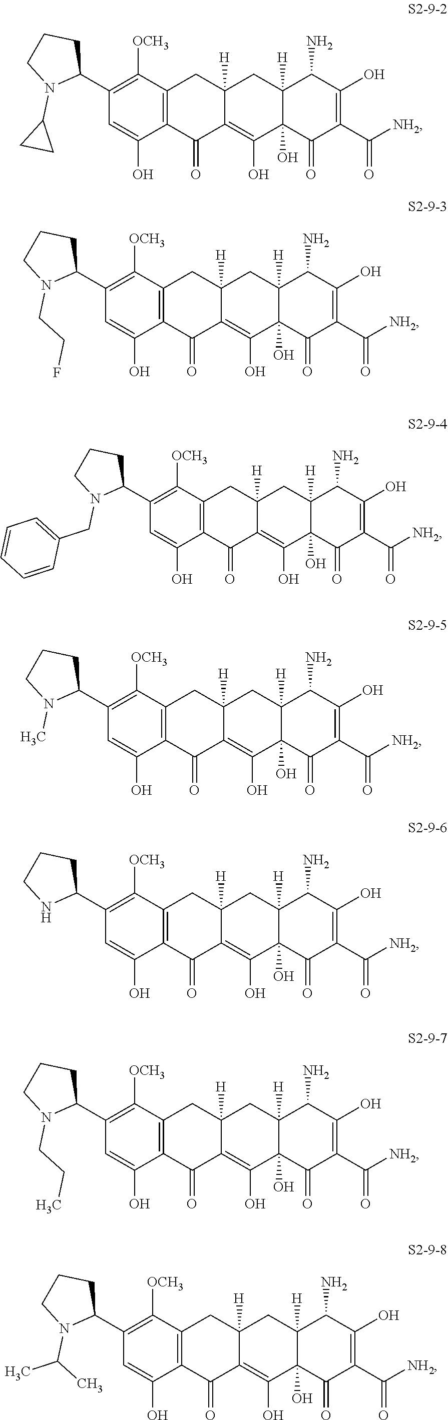

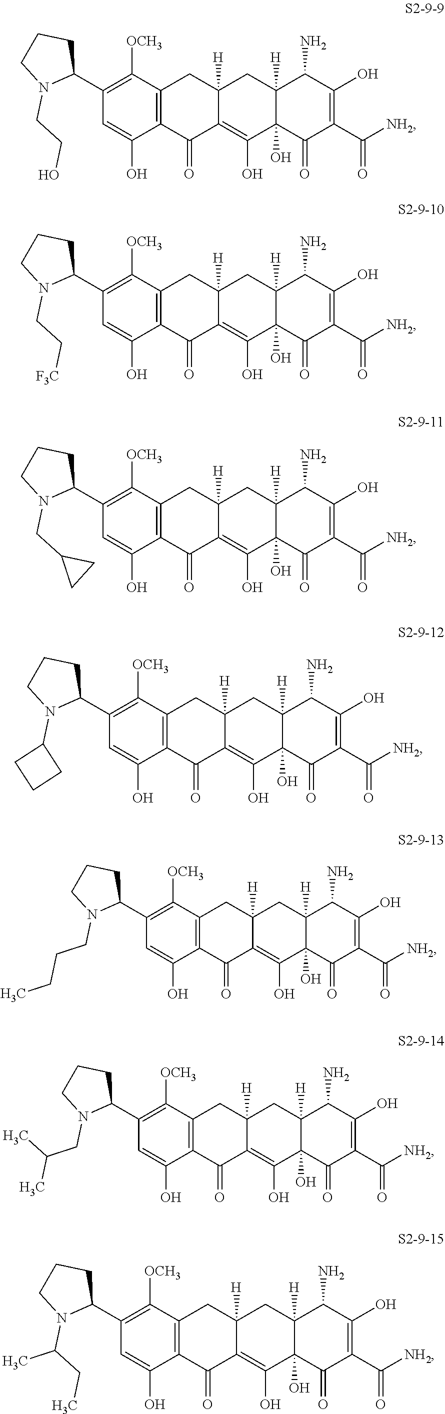

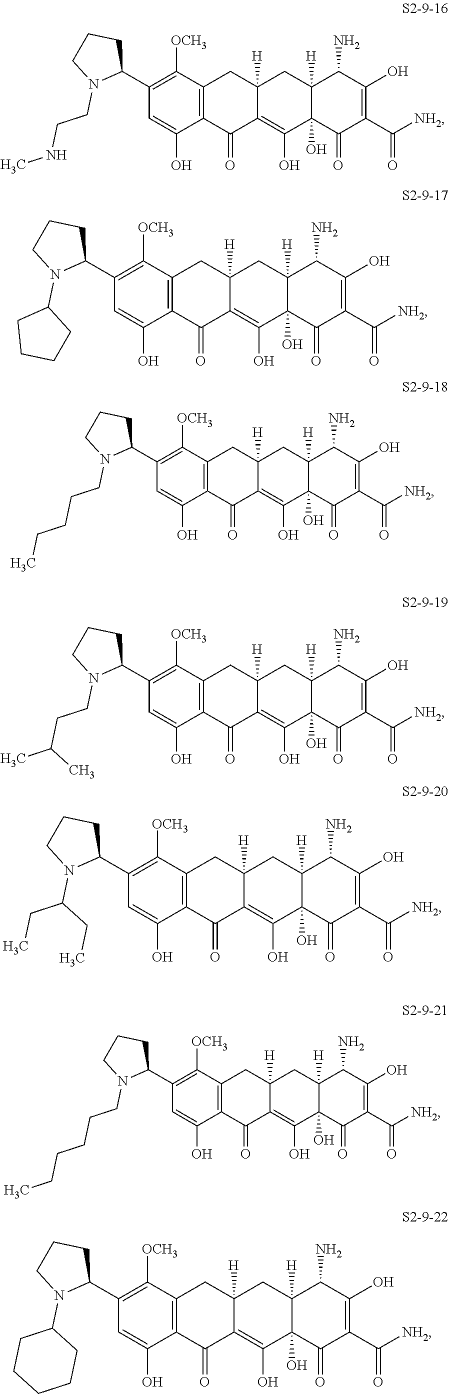

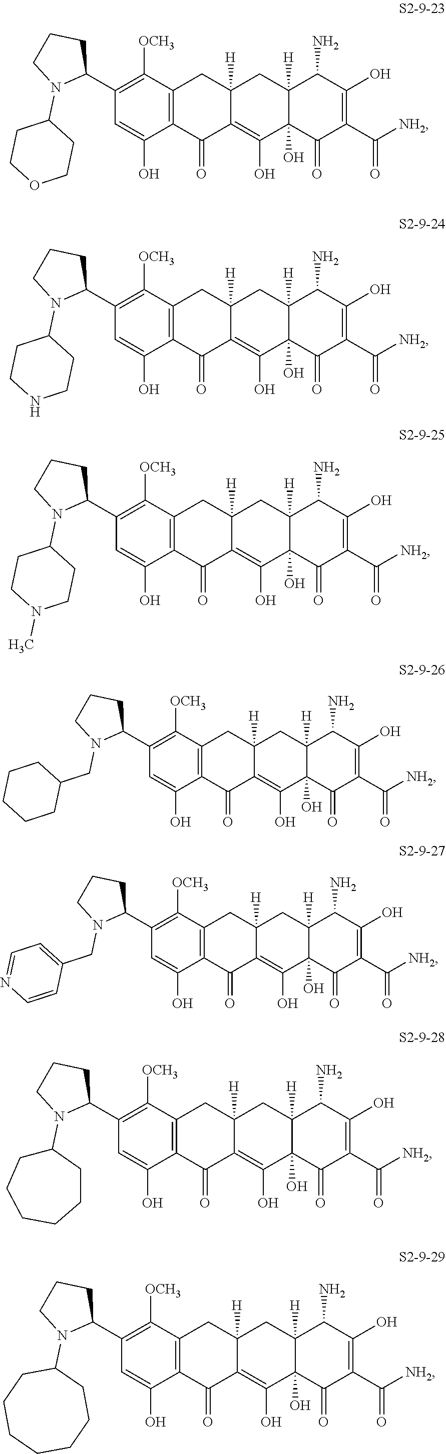

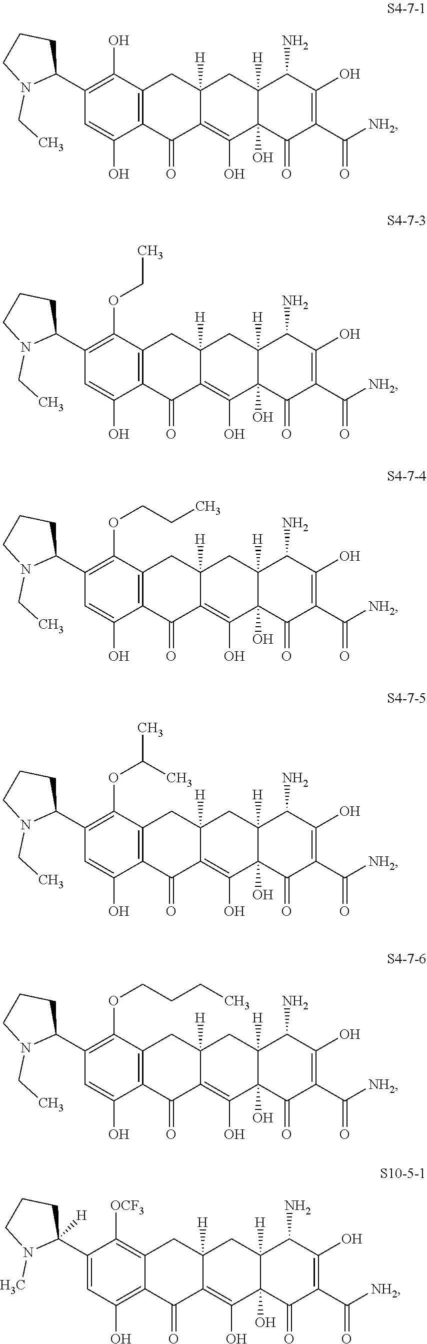

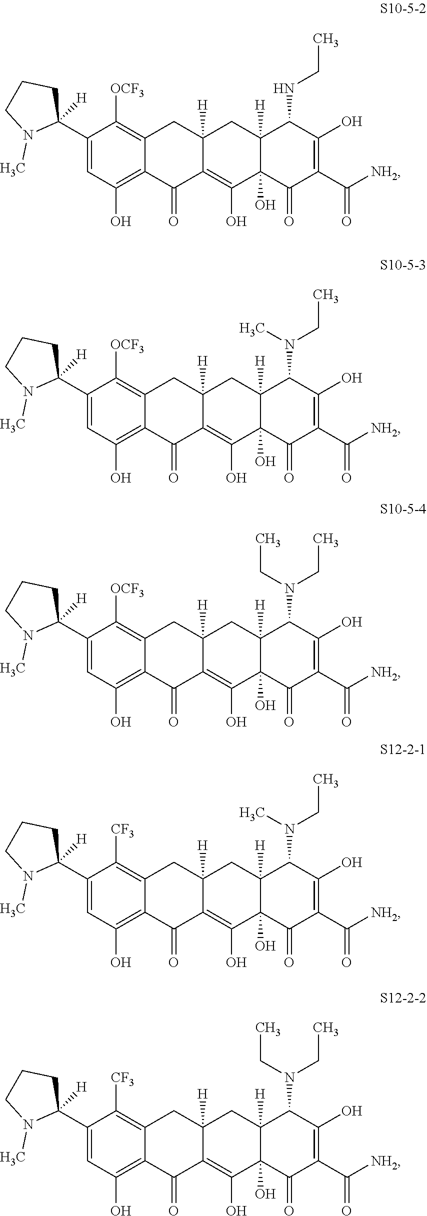

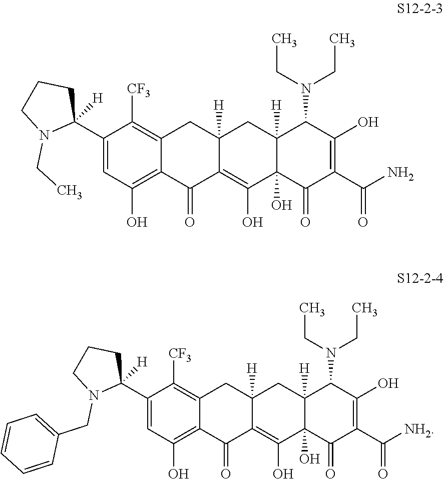 [SMILES: CCN(CC)[C@@H]1C(O)=C(C(N)=O)C(=O)[C@@]2(O)C(O)=C3C(=O)c4c(O)cc([C@@H]5CCCN5CC)c(C(F)(F)F)c4C[C@H]3C[C@@H]12 ; CCN(CC)[C@@H]1C(O)=C(C(N)=O)C(=O)[C@@]2(O)C(O)=C3C(=O)c4c(O)cc([C@@H]5CCCN5Cc5ccccc5)c(C(F)(F)F)c4C[C@H]3C[C@@H]12]

140. The method according to claim 52, wherein the compound is represented by any one of the following structural formulas, or a pharmaceutically acceptable salt thereof:

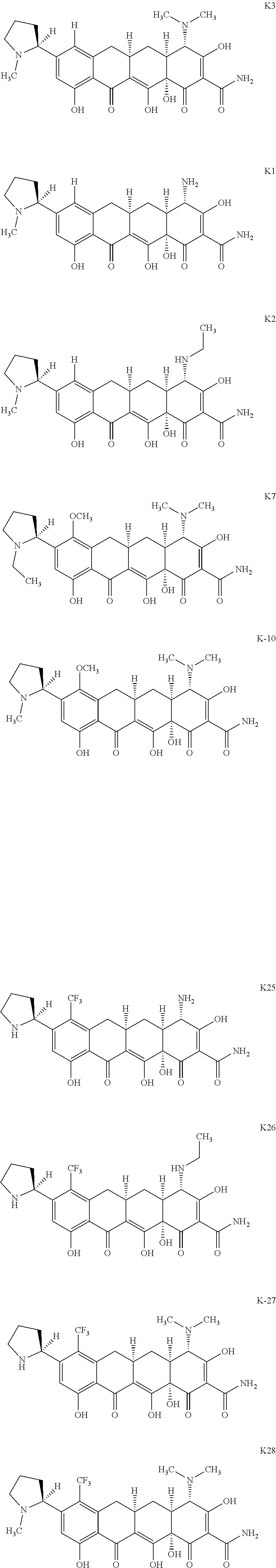

141. The method according to claim 52, wherein the method comprises administration of one or more additional therapeutic agents, wherein the additional therapeutic agents are cytarabine and an anthracycline drug.

142. The method according to claim 141, wherein the anthracycline drug is selected from daunorubicin or idarubicin.

143. The method according to claim 141, wherein the method further includes administration of cladribine.

144. The method according to claim 52, wherein the subject is a human.

145. The method according to claim 52, wherein the compound is represented by the following structural formula 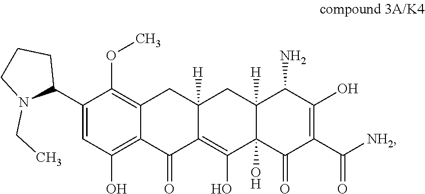
or a pharmaceutically acceptable salt thereof.
